(19) United States
(12) Patent Application Publication
(10) Pub. No.: US 2026/0200928 A1
(43) Pub. Date: Jul. 16, 2026

(54) THERAPEUTIC COMPOUNDS

(71) Applicant: Sanofi, Paris (FR)

(72) Inventors: Christian BUNING, Frankfurt am Main (DE); Nis HALLAND, Frankfurt am Main (DE); Uwe HEINELT, Frankfurt am Main (DE); Hans MATTER, Frankfurt am Main (DE); Ian Duncan LINNEY, Manchester (GB); Jan-Christoph WESTERMANN, Manchester (GB)

(21) Appl. No.: 19/136,591

(22) PCT Filed: Dec. 8, 2023
(86) PCT No.: PCT/EP2023/085000

§ 371 Date: Jun. 6, 2025

(30) Foreign Application Priority Data
Dec. 9, 2022 (EP) 22212666.6

(51) Int. Cl. C07D 487/04; A61K 31/4439; A61K 31/444; A61K 31/497; A61K 31/4985; A61K 31/506; A61K 31/513; C07B 59/00; C07D 213/75; C07D 401/06; C07D 401/12; C07D 401/14; C07D 413/12; C07D 413/14; C07D 417/14

(52) U.S. Cl. CPC C07D 487/04; A61K 31/4439; A61K 31/444; A61K 31/497; A61K 31/4985; A61K 31/506; A61K 31/513; C07B 59/002; C07D 213/75; C07D 401/06; C07D 401/12; C07D 401/14; C07D 413/12; C07D 413/14; C07D 417/14; C07B 2200/05

(57) ABSTRACT

The present disclosure relates to compounds that are IL-17A modulators. The compounds have the structural Formula I defined herein. The present disclosure also relates to processes for the preparation of these compounds, to pharmaceutical compositions comprising them, and to their use in the treatment of diseases or disorders associated with modulation of IL-17A activity.

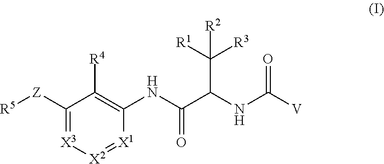

### CROSS REFERENCE TO RELATED APPLICATIONS

This application is a U.S. national stage application under 35 U.S.C. § 371 of International Application No. PCT/EP2023/085000, filed internationally on Dec. 8, 2023, which claims priority to EP Application No. 22212666.6, filed on Dec. 9, 2022, each of which is incorporated herein by reference in their entirety.

### INTRODUCTION

The present disclosure relates to therapeutic compounds. More specifically, the present disclosure relates to compounds that are modulators of IL-17A activity. The present disclosure also relates to processes for the preparation of these compounds, to pharmaceutical compositions comprising them, and to their use in the treatment of diseases or disorders associated with IL-17A activity.

### BACKGROUND

The interleukin-17 cytokine family consists of six members (termed IL-17A through IL-17F) of which IL-17A (also known as CTLA-8) is the primary effector cytokine of the T-helper-17 (Th17) cell lineage.

IL-17A is a variably glycosylated, disulfide linked, homodimeric glycoprotein of 34-38 kDa which shares in the order of 50% homology with its closest family member IL-17F, both of which can be secreted either as homodimers or the heterodimer IL-17AF [K. F. Geoghegan et al., Protein Expression and Purification 2013, 87, 27-34; J. K. Kolls and A. Lindén/Immunity 2004, 21, 467-476].

Activation of naïve CD4+ T-cells in response to cytokines such as IL-6, transforming growth factor β (TGF-β), IL-23, STAT3, and RORγt leads to their differentiation to TH17 cells and expression of pro-inflammatory mediators such as IL-17A. Furthermore, a variety of cell types from the innate and adaptive immune systems have been identified as sources of IL-17A. These include mast cells, neutrophilic granulocytes, NK cells, NKT cells, CD8+ T cells, δγ T-cells, macrophages, and type 3-innate lymphoid cells [D. J. Cua and C. M. Tato, Nat Rev Immunol 2010, 10, 479-489; W. Jin and C. Dong, Emerging Microbes & Infections 2013, 2, e60].

Cytokines IL-17A, IL-17F, and IL-17AF bind to common heteromeric receptor complexes IL-17RA and IL-17RC, albeit with different affinities, and although various cell types have been reported to express the IL-17RA subunit, the highest responses to IL-17A come from epithelial cells, endothelial cells, keratinocytes, and fibroblasts [T. A. Moseley et al., Cytokine Growth Factor Reviews. 2003, 14, 155-174; S. L. Gaffen, Nature Rev Immunol 2009, 9, 556-567; R. M. Onishi and S. L. Gaffen, Immunology 2010, 129, 311-321].

Binding of IL-17A to its receptor activates various signal transduction pathways such as nuclear factor (NF)-κB, phosphoinositide 3-kinase (PI3K), activator protein (AP1), CCAAT/enhancer-binding protein (C/EBP), and mitogen-activated protein kinase (MAPK) leading to pro-inflammatory gene expression and the secretion of various pro-inflammatory cytokines including IL-1β, IL-6, IL-8, TNFα, G-CSF, PGE2, and IFN-γ as well as numerous chemokines and other effectors [S. L. Gaffen, Arthritis Research & Therapy 2004, 6, 240-247; S. L. Gaffen, Nature Rev Immunol 2009, 9, 556-567; R. M. Onishi and S. L. Gaffen, Immunology 2010, 129, 311-321]. The attraction and activation of cells of the innate immune system to the site of inflammation completes the induction of an inflammatory loop which may also be mediated cooperatively with other cytokines such as TNFα, IFN-γ, and IL-1β [S. L. Gaffen, Arthritis Research & Therapy 2004, 6, 240-247].

These IL-17 mediated biological processes have been implicated in the pathology of many human diseases with an immune component or autoimmune pathology, such as psoriasis, ankylosing spondylitis, axial spondyloarthritis, psoriatic arthritis, eczema, enthesitis-related arthritis, asthma (including severe asthma), chronic obstructive pulmonary disease (COPD), cystic fibrosis, pulmonary fibrosis, ulcerative colitis, Crohn's disease, atopic dermatitis, contact dermatitis, dermatomyositis, myocarditis, uveitis, exophtalmos, autoimmune thyroiditis, Peyronie's disease, coeliac disease, gall bladder disease, Pilonidal disease, peritonitis, multiple sclerosis, Guillan-Bar Syndrome, irritable bowel syndrome, inflammatory bowel disease, Castleman's disease, pelvic inflammatory disease, systemic onset juvenille idiopathic arthritis (JIA), rheumatoid arthritis, giant cell arteritis, graft versus host disease, discoid lupus erythematosus, systemic lupus erythematosus, lupus nephritis, vasculitis, insulin dependent diabetes type I, autoimmune diabetes, Necrobiosis Lipoidica Diabeticorum, Pyoderma Gangrenosum, Hidradenitis Suppurativa, Papulopustular Rosacea, Lichen Planus (Solimani et al (2019) Front Immunol. 10:1808), and also in cancer (Bartlett, HS; Million, RP (2015) Nat. Rev. Drug Discovery 14:11-12; Santibanez, J F; Bjelica, S (2018) Recent Pat Anticancer Drug Discov. 13(2):133-144). In addition, due to the emerging role of neuroinflammation in neurodegeneration, IL-17 has also been implicated in the progression of neurodegenerative disorders such as Alzheimer's disease (Cristiano et al (2019) Br J Pharmacol. 176(18):3544-3557) and Parkinson's disease (Storelli et al, (2019) Front Neurol. 24; 10:13). An association between serum levels of IL-17 at the time of admission to the intensive care unit and the development of sepsis has also been observed suggesting increased IL-17 may increase the susceptibility for septic complications and endotoxic shock associated with infection [Ahmed et al., Eur J Trauma Emerg Surg 2018, 44(4):621-626]. Its role in sepsis has also been suggested to extend to patients with sepsis-induced Acute Respiratory Distress Syndrome (ARDS) and acute lung injury [Ding et al., Oncotarget 2017, 8(55):93704-93711]. Recently inhibition of IL-17 has also been suggested to be used to prevent acute respiratory distress syndrome (ARDS) in coronavirus disease 2019 (COVID-19) [Pacha, Sallman & Evans., Nat Rev Immunol 2020, 1:1-2].

Pre-clinical studies have demonstrated that IL-17A (as well as IL-17F and IL-17C) is elevated in psoriatic skin [N. J. Wilson et al., Nat Immunol 2007, 8, 950-957; L. C. Zaba et al., J Exp Med 2007, 204, 3183-3194; C. Ortega et al, J Leukocyte Biol 2009, 86, 435-443; C. Johansen et al., Br J Dermatol 2009, 160, 319-324]. Th17 cells in the peripheral circulation and lesional skin of patients with psoriasis have also been shown to positively correlate with disease severity as measured by the Psoriasis Area and Severity Index (PASI) score [L. Zhang et al., Clin Immunol 2010, 135, 108-117]. Serum IL-17A levels are also significantly correlated with PASI score [H. Takahashi et al., Clin Exp Dermatol 2010, 35, 645-649; S. B. Yilmaz et al. Arch Dermatol Res 2012, 304, 465-469; M. Caproni et al., J Clin Immunol 2009, 29, 210-214].

Animal model studies supported the hypothesis that targeting the IL-17A pathway would be an effective treatment for psoriasis [L. van der Fits et al., J Immunol 2009, 182, 5836-5845; K. El Malki et al., J Investig Dermatol 2013, 133, 441-451; J. Skepner et al., J Immunol 2014, 192, 2564-2575], and clinical results with antibodies to IL-17A or IL-17RA delivered the ultimate validation with excellent efficacy being observed [R. G. Langley et al., N Engl J Med 2014, 371, 326-338; K. B. Gordon et al., N Engl J Med 2016, 375, 345-356; A. S. Lonnberg et al., Clin Cosmet Investig Dermatol 2014, 7, 251-259; S. Coimbra et al., Core Evid 2014, 9, 89-97; M. Lebwohl et al., N Engl J Med 2015, 373, 1318-1328].

Elevated levels of IL-17A or IL-17F have been reported in a number of other diseases including Rheumatoid Arthritis (RA), Psoriatic Arthritis (PsA), Ankylosing Spondylitis (AS), Systemic Lupus Erythematosus (SLE), Inflammatory Bowel Disease (IBD), Multiple Sclerosis (MS), bone erosion, intraperitoneal abscesses, allograft rejection, angiogenesis, atherosclerosis, and asthma [e.g., S. L. Gaffen, Arthritis Research & Therapy 2004, 6, 240-247; L. A. Tesmer et al., Immunol Rev 2008, 223, 87-113; US Publ No 20080269467].

The anti-IL-17A therapeutic antibodies Secukinumab and Ixekizumab have shown evidence of positive effects in treating palmoplantar and nail psoriasis; [A. Gottlieb et al., J Am Acad Dermatol 2016, 76, 70-80; A. Menter et al., J Eur Acad Dermatol Venereol 2017, 31, 1686-1692; C. Paul et al., J Eur Acad Dermatol Venereol 2014, 28, 1670-1675]; PsA [P. Mease et al., Ann Rheum Dis 2018, 77, 890-897; P. Nash et al., Lancet 2017, 389, 2317-2327] and AS [K. Pavelka et al., Arthritis Res Ther 2017, 19, 285; A. Deodhar et al., Arthritis Rheumatol 2018, doi:10.1002/art.40753]. A proof of concept study with Secukinumab in MS has also shown encouraging signs of efficacy [E. Havdrova et al., J Neurol 2016, 263, 1287-1295].

IL-17A expression has been shown to be increased in SLE patients and correlated with disease severity [Y. Wang et al., Clin Exp Immunol 2009, 159, 1-10; X. Q. Chen et al., J Clin Immunol 2010, 30, 221-225].

In addition, IL-17A has been associated with ocular surface disorders such as DES [PCT publications WO2009089036, WO2010062858 and WO2011163452; C. S. De Paiva et al., Mucosal Immunol 2009, 2, 243-253] and Th17 cells have been shown to be elevated in active uveitis and scleritis [A. Amadi-Obi et al., Nat Med 2007, 13, 711-718]. IL-17A levels in tears were associated with clinical severity of dry eye in patients with a range of systemic autoimmune or inflammatory diseases including Sjögren's syndrome, Stevens-Johnson syndrome (SJS), SLE, filamentary keratitis, DES, Meibomian gland dysfunction (MGD), and Graft-versus-Host disease (GVHD) [M. H. Kang et al., J Korean Med Sci 2011, 26, 938-944].

Several studies have demonstrated that IL-17A is overexpressed in patients with a range of cancers including gastric carcinoma, medulloblastoma, multiple myeloma, colorectal carcinoma, Non-Small-Cell Lung Cancer (NSCLC), breast cancer, hepatocellular carcinoma (HCC), and thyroid cancer [X. Meng et al., Turk J Gastroenterol 2018, 29, 45-51; P. Zhou et al., J Int Med Res 2010, 38, 611-619; D. Lemancewicz et al., Med Sci Monit 2012, 18, BR 54-59; S. Le Gouvello et al., Gut 2008, 57, 772-779; B. Pan et al., Sci Rep 2015, 5, 16053; T. Welte and X. H-F. Zhang, Mediators Inflammation 2015, 804347; J-F. Tu et al., Medicine (Baltimore) 2016, 95, e3220; D. F. G. Carvalho et al., Oncol Lett 2017, 13, 1925-1931]. Increased levels of IL-17A have been shown to correlate with poor prognosis in several cancer types including malignant thyroid tumor, breast cancer, pancreatic carcinoma, gastric cancer, NSCLC, colorectal cancer, and head and neck cancer [S. Punt et al., Oncolmmunol 2015, 4, e984547; D. F. G. Carvalho et al., Oncol Lett 2017, 13, 1925-1931; W-C. Chen et al., Histopathology 2013, 63, 225-233; C. Xu et al., Biomarkers 2014, 19, 287-290; Y. Yamada et al., J Surg Res 2012, 178, 685-691; S. He et al., Int J Mol Sci 2011, 12, 7424-7437; J-Y. Tseng et al., Clin Cancer Res 2014, 20, 2885-2897; M-H. Lee et al., Oncotarget 2018, 9, 9825-9837].

Taken together, modulation of the IL-17A pathway, in particular modulation of IL-17A activity through inhibition of its interaction with the receptor IL-17RA, may be considered a target for the treatment of conditions relating to the immune system and inflammation, cancer, and neurodegenerative disorders.

WO 2013/116682, WO 2014/066726, and WO 2018/229079 describe classes of chemical compounds that are stated to modulate the activity of IL-17 and to be useful in the treatment of medical conditions, including inflammatory disease.

Nevertheless, there is an ongoing need for compounds capable of attenuating IL-17A activity.

### SUMMARY

In one aspect, the present disclosure provides a compound, or a pharmaceutically acceptable salt thereof as defined herein.

In another aspect, the present disclosure provides a pharmaceutical composition comprising a compound of the disclosure as defined herein, or a pharmaceutically acceptable salt thereof, and one or more pharmaceutically acceptable excipients.

In another aspect, the present disclosure relates to a compound of the disclosure as defined herein, or a pharmaceutically acceptable salt thereof, or a pharmaceutical composition as defined herein, for use in therapy.

In another aspect, the present disclosure relates to a compound of the disclosure as defined herein, or a pharmaceutically acceptable salt thereof, or a pharmaceutical composition as defined herein, for use in the treatment of diseases or disorders associated with IL-17A activity.

In another aspect, the present disclosure relates to the use of a compound of the disclosure as defined herein, or a pharmaceutically acceptable salt thereof, in the manufacture of a medicament for use in the treatment of diseases or disorders associated with IL-17A activity.

In another aspect, the present disclosure relates to a method of treating a disease or disorder associated with IL-17A activity, said method comprising administering to a subject in need of such treatment a therapeutically effective amount of a compound of the disclosure as defined herein, or a pharmaceutically acceptable salt thereof, or a pharmaceutical composition as defined herein.

Examples of diseases or disorders associated with IL-17A activity include diseases with an immune component or autoimmune pathology (such as psoriasis, ankylosing spondylitis, psoriatic arthritis, and rheumatoid arthritis), cancer, and neurodegenerative disorders.

In another aspect, the present disclosure provides a compound, or a pharmaceutically acceptable salt thereof, or a pharmaceutical composition as defined herein, for use in the treatment of diseases with an immune component or autoimmune pathology (such as psoriasis, ankylosing spondylitis, psoriatic arthritis, and rheumatoid arthritis), cancer, and neurodegenerative disorders.

In another aspect, the present disclosure provides the use of a compound, or a pharmaceutically acceptable salt, in the manufacture of a medicament for use in the treatment of diseases with an immune component or autoimmune pathology (such as psoriasis, ankylosing spondylitis, psoriatic arthritis, and rheumatoid arthritis), cancer, and neurodegenerative disorders.

In another aspect, the present disclosure provides a method of treating diseases with an immune component or autoimmune pathology (such as psoriasis, ankylosing spondylitis, psoriatic arthritis, and rheumatoid arthritis), cancer, and neurodegenerative disorders, said method comprising administering to a subject in need of such treatment a therapeutically effective amount of a compound, or a pharmaceutically acceptable salt thereof, or a pharmaceutical composition as defined herein.

The present disclosure further provides a method of synthesising a compound, or a pharmaceutically acceptable salt thereof, as defined herein.

In another aspect, the present disclosure provides a compound, or a pharmaceutically acceptable salt thereof, obtainable by, or obtained by, or directly obtained by a method of synthesis as defined herein.

In another aspect, the present disclosure provides novel intermediates as defined herein which are suitable for use in any one of the synthetic methods set out herein.

Convenient, suitable, and optional features of any one particular aspect of the present disclosure are also convenient, suitable, and optional features of any other aspect.

### DETAILED DESCRIPTION

### Definitions

Unless otherwise stated, the following terms used in the specification and claims have the following meanings set out below.

It is to be appreciated that references to “treating” or “treatment” include prophylaxis as well as the alleviation of established symptoms of a condition. “Treating” or “treatment” of a state, disorder, or condition therefore includes: (1) preventing or delaying the appearance of clinical symptoms of the state, disorder, or condition developing in a human that may be afflicted with or predisposed to the state, disorder, or condition but does not yet experience or display clinical or subclinical symptoms of the state, disorder, or condition, (2) inhibiting the state, disorder, or condition, i.e., arresting, reducing, or delaying the development of the disease or a relapse thereof (in case of maintenance treatment) or at least one clinical or subclinical symptom thereof, or (3) relieving or attenuating the disease, i.e., causing regression of the state, disorder, or condition or at least one of its clinical or subclinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity, and the age, weight, etc., of the mammal to be treated.

In this specification the term “alkyl” refers to aliphatic hydrocarbon groups and includes both straight and branched chain alkyl groups. References to individual alkyl groups such as “propyl” are specific for the straight chain version only and references to individual branched chain alkyl groups such as “isopropyl” are specific for the branched chain version only. For example, “C₁₋₆alkyl” includes C₁₋₄alkyl, C₁₋₃alkyl, propyl, isopropyl, and t-butyl. A similar convention applies to other radicals, for example “phenylC₁₋₆alkyl” includes phenylC₁₋₄alkyl, benzyl, 1-phenylethyl, and 2-phenylethyl.

The term “alkylene” includes both straight and branched chain divalent alkyl groups. For example, “C₁₋₄alkylene” comprises methylene (—CH₂—), ethylene (—CH₂CH₂—), methyl methylene (—CH(CH₃)—), propylene, and butylene.

The term “alkoxy” includes both straight and branched chain alkyl groups singularly bonded to oxygen. For example, “C₁₋₄alkoxy” comprises methoxy, ethoxy, isopropoxy, and t-butoxy.

The term “Cₘ₋ₙ” used as a prefix, refers to any group having m to n carbon atoms.

“Cycloalkyl” means a hydrocarbon ring containing from 3 to 8 carbon atoms, for example, cyclopropyl, cyclobutyl, cyclopentyl, cyclohexyl, cycloheptyl, bicycle[2.2.2]octane, bicycle[2.1.1]hexane, bicycle[1.1.1]pentane, and bicyclo[2.2.1]heptyl.

The term “halo” refers to fluoro, chloro, bromo, or iodo.

The term “haloalkyl” or “haloalkoxy” is used herein to refer to an alkyl or alkoxy group respectively in which one or more hydrogen atoms have been replaced by halogen (e.g., fluorine) atoms. Examples of haloalkyl and haloalkoxy groups include fluoroalkyl and fluoroalkoxy groups such as —CHF₂, —CH₂CF₃, or perfluoroalkyl/alkoxy groups such as —CF₃, —CF₂CF₃, or —OCF₃.

The term “heterocyclyl”, “heterocyclic”, or “heterocycle” means a non-aromatic saturated or partially unsaturated monocyclic, fused, bridged, or spiro bicyclic heterocyclic ring system(s). Monocyclic heterocyclic rings contain from about 3 to 12 (suitably from 3 to 7) ring atoms, with from 1 to 5 (suitably 1, 2 or 3) heteroatoms selected from nitrogen, oxygen, or sulfur in the ring. Bicyclic heterocycles contain from 7 to 17 member atoms, suitably 7 to 12 member atoms, in the ring. Bicyclic heterocyclic(s) rings may be fused, spiro, or bridged ring systems. Examples of heterocyclic groups include cyclic ethers such as oxiranyl, oxetanyl, tetrahydrofuranyl, dioxanyl, and substituted cyclic ethers. Heterocycles containing nitrogen include, for example, azetidinyl, pyrrolidinyl, piperidinyl, piperazinyl, tetrahydrotriazinyl, tetrahydropyrazolyl, and the like. Typical sulfur containing heterocycles include tetrahydrothienyl, dihydro-1,3-dithiol, tetrahydro-2H-thiopyran, and hexahydrothiepine. Other heterocycles include dihydro-oxathiolyl, tetrahydro-oxazolyl, tetrahydro-oxadiazolyl, tetrahydrodioxazolyl, tetrahydro-oxathiazolyl, hexahydrotriazinyl, tetrahydro-oxazinyl, morpholinyl, thiomorpholinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothienyl and thiomorpholinyl such as tetrahydrothiene 1,1-dioxide and thiomorpholinyl 1,1-dioxide. A suitable value for a heterocyclyl group which bears 1 or 2 oxo (═O) or thioxo (═S) substituents is, for example, 2-oxopyrrolidinyl, 2-thioxopyrrolidinyl, 2-oxoimidazolidinyl, 2-thioxoimidazolidinyl, 2-oxopiperidinyl, 2,5-dioxopyrrolidinyl, 2,5-dioxoimidazolidinyl, or 2,6-dioxopiperidinyl. Particular heterocyclyl groups are saturated monocyclic 3 to 7 membered heterocyclyls containing 1, 2, or 3 heteroatoms selected from nitrogen, oxygen, or sulfur, for example azetidinyl, tetrahydrofuranyl, tetrahydropyranyl, pyrrolidinyl, morpholinyl, tetrahydrothienyl, tetrahydrothienyl 1,1-dioxide, thiomorpholinyl, thiomorpholinyl 1,1-dioxide, piperidinyl, homopiperidinyl, piperazinyl, or homopiperazinyl. Partially unsaturated heterocyclyl rings contain at least one double bond, such as 1 or 2 double bonds. Examples of partially unsaturated heterocyclyl rings include 1,6-dihydropyridinyl, 1,6-dihydropyridazinyl, and 2,3-dihydropyrrolyl. As the skilled person would appreciate, any heterocycle may be linked to another group via any suitable atom, such as via a carbon or nitrogen atom. Suitably, the term “heterocyclyl”, “heterocyclic”, or “heterocycle” will refer to 4, 5, 6, or 7 membered monocyclic rings as defined above.

The term “bridged ring systems” means ring systems in which two rings share more than two atoms, see for example Advanced Organic Chemistry, by Jerry March, 4th Edition, Wiley Interscience, pages 131-133, 1992. Examples of bridged heterocyclyl ring systems include, for example azabicyclo[3.1.0]hexane, aza-bicyclo[2.2.1]heptane, 2-oxa-5-azabicyclo[2.2.1]heptane, aza-bicyclo[2.2.2]octane, aza-bicyclo[3.2.1]octane, and quinuclidine.

The term “spiro bi-cyclic ring systems” means that the two ring systems share one common spiro carbon atom, i.e., the heterocyclic ring is linked to a further carbocyclic or heterocyclic ring through a single common spiro carbon atom. Examples of spiro bi-cyclic ring systems include 6-azaspiro[3.4]octane, 2-oxa-6-azaspiro[3.4]octane, 2-azaspiro[3.3]heptanes, and 2-oxa-6-azaspiro[3.3]heptanes.

The term “heteroaryl” or “heteroaromatic” means an aromatic mono-, bi-, or polycyclic ring incorporating one or more (for example 1-4, particularly 1, 2, or 3) heteroatoms selected from nitrogen, oxygen, or sulfur. Examples of heteroaryl groups are monocyclic and bicyclic groups containing from five to twelve ring members, and more usually from five to ten ring members. The heteroaryl group can be, for example, a 5- or 6-membered monocyclic ring or a 9- or 10-membered bicyclic ring, for example a bicyclic structure formed from fused 5 and 6 membered rings or two fused 6 membered rings. Each ring may contain up to about four heteroatoms typically selected from nitrogen, sulfur, and oxygen. Typically, the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general, the number of basic nitrogen atoms present in the heteroaryl group, including any amino group substituents of the ring, will be less than five. Heteroaryl groups containing nitrogen atoms may be present as the corresponding N-oxides. Particular examples of such heteroaryl groups are pyridine N-oxides. Suitably, the term “heteroaryl” or “heteroaromatic” will refer to 5- or 6-membered monocyclic heteroaryl rings as defined above.

Non-limiting examples of heteroaryl groups include oxazolyl, isoxazolyl, imidazolyl, pyrazolyl, thiazolyl, oxadiazolyl, tetrazolyl, pyridyl, and pyrimidinyl groups.

Non-limiting examples of 5 membered heteroaryl groups include but are not limited to imidazolyl, oxazolyl, oxadiazolyl, isoxazolyl, thiazolyl, pyrazolyl, and tetrazolyl groups.

Non-limiting examples of 6 membered heteroaryl groups include but are not limited to pyridyl, and pyrimidinyl groups.

A bicyclic heteroaryl group may be, for example, a group selected from:
a benzene ring fused to a 5- or 6-membered ring containing 1, 2, or 3 ring heteroatoms;
a pyridine ring fused to a 5- or 6-membered ring containing 1, 2, or 3 ring heteroatoms;
a pyrimidine ring fused to a 5- or 6-membered ring containing 1 or 2 ring heteroatoms;
a pyrrole ring fused to a 5- or 6-membered ring containing 1, 2, or 3 ring heteroatoms;
a pyrazole ring fused to a 5- or 6-membered ring containing 1 or 2 ring heteroatoms;
a pyrazine ring fused to a 5- or 6-membered ring containing 1 or 2 ring heteroatoms;
an imidazole ring fused to a 5- or 6-membered ring containing 1 or 2 ring heteroatoms;
an oxazole ring fused to a 5- or 6-membered ring containing 1 or 2 ring heteroatoms;
an isoxazole ring fused to a 5- or 6-membered ring containing 1 or 2 ring heteroatoms;
a thiazole ring fused to a 5- or 6-membered ring containing 1 or 2 ring heteroatoms;
an isothiazole ring fused to a 5- or 6-membered ring containing 1 or 2 ring heteroatoms;
a thiophene ring fused to a 5- or 6-membered ring containing 1, 2, or 3 ring heteroatoms;
a furan ring fused to a 5- or 6-membered ring containing 1, 2, or 3 ring heteroatoms;
a cyclohexyl ring fused to a 5- or 6-membered heteroaromatic ring containing 1, 2, or 3 ring heteroatoms; and
a cyclopentyl ring fused to a 5- or 6-membered heteroaromatic ring containing 1, 2, or 3 ring heteroatoms.

Particular non-limiting examples of bicyclic heteroaryl groups containing a six membered ring fused to a 5 membered ring include but are not limited to benzofuranyl, benzothiophenyl, benzimidazolyl, benzoxazolyl, benzisoxazolyl, benzothiazolyl, benzisothiazolyl, isobenzofuranyl, indolyl, isoindolyl, indolizinyl, indolinyl, isoindolinyl, purinyl (e.g., adeninyl, guaninyl), indazolyl, benzodioxolyl, pyrrolopyridine, and pyrazolopyridinyl groups.

Particular non-limiting examples of bicyclic heteroaryl groups containing two fused 6 membered rings include but are not limited to quinolinyl, isoquinolinyl, chromanyl, thiochromanyl, chromenyl, isochromenyl, chromanyl, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl, and pteridinyl groups.

The term “aryl” means a cyclic or polycyclic aromatic ring having from 5 to 12 carbon atoms. The term aryl includes both monovalent species and divalent species. Examples of aryl groups include, but are not limited to, phenyl, biphenyl, naphthyl, and the like. In an embodiment, an aryl is phenyl or naphthyl. In another embodiment, an aryl is phenyl.

This specification may also make use of several composite terms to describe groups comprising more than one functionality. Such terms will be understood by a person skilled in the art. For example, heterocyclylC₁₋₄alkyl comprises C₁₋₄alkyl substituted by heterocyclyl.

The term “optionally substituted” refers to either groups, structures, or molecules that are substituted and those that are not substituted.

Where optional substituents are chosen from “one or more” groups it is to be understood that this definition includes all substituents being chosen from one of the specified groups or the substituents being chosen from two or more of the specified groups. It is understood that where there are multiple substituents, the substituents chosen may be the same or different.

Where numerical ranges are given, it is understood that the ranges are inclusive of the endpoints.

The phrase “compound of the disclosure” means those compounds which are disclosed herein, both generically and specifically.

### Compounds of the Disclosure

In a first aspect, the present disclosure provides a compound of Formula I, or a pharmaceutically acceptable salt thereof:

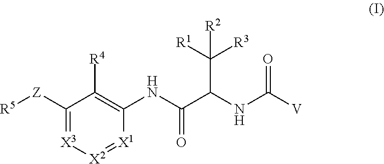

wherein:
Z is O or CR⁶R⁷;
at least one of X¹, X², and X³ is N and the others are CR⁴;
Y is selected from 5- or 6-membered monocyclic heteroaryl, 9- or 10-membered bicyclic heteroaryl, 5- or 6-membered heteroaryl fused to 5- or 6-membered cycloalkyl or 5- or 6-membered heterocyclyl, phenyl, heterocyclyl, C₃₋₇cycloalkyl, and QR⁸; wherein:
   i. Q is absent, O, or NR⁹;
   ii. R⁸ is C₁₋₆alkyl, C₁₋₃alkylene-phenyl, or C₁₋₃alkylene-heteroaryl;
   iii. Y is optionally substituted with one or more substituents Y^{A}, wherein at each occurrence Y^{A} is independently selected from halo, oxo, C₁₋₄alkyl, hydroxy, C₁₋₄alkoxy, C₁₋₃haloalkyl, cyano, C(O)R¹⁰, and C₃₋₇cycloalkyl; and
   iv. Y^{A} is optionally further substituted with one or more substituents independently selected from halo, C₁₋₄alkyl, hydroxy, C₁₋₄alkoxy, C₃₋₇cycloalkyl, C₁₋₃alkylene-NR¹¹R¹², and C₁₋₃haloalkyl;
R¹ and R² are independently selected from hydrogen, C₁₋₄alkyl, C₃₋₇cycloalkyl, C₁₋₅alkoxy, and phenyl, wherein said C₁₋₄alkyl, C₃₋₇cycloalkyl, C₁₋₅alkoxy, or phenyl is optionally substituted with one or more substituents independently selected from halo, C₁₋₄alkyl, and C₁₋₃haloalkyl; provided that R¹ and R² cannot be both hydrogen; OR
R¹ and R², taken together with the carbon atom to which they are attached, form a 4- to 10-membered cycloalkyl ring, wherein the cycloalkyl ring is optionally substituted with one or more substituents independently selected from halo, C₁₋₄alkyl, and C₁₋₃haloalkyl;
R³ is hydrogen, fluoro, or methyl;
R⁴ at each occurrence is independently selected from hydrogen, fluoro, methyl, hydroxy, and trifluoromethyl;
R⁵ is selected from 5- or 6-membered monocyclic heteroaryl, 4- to 10-membered heterocyclyl, C(O)NR¹³R¹⁴, OC(O)NR¹⁵R¹⁶, and NR¹⁷C(O)C₁₋₆alkyl, wherein R¹³, R¹⁴, R¹⁵, R¹⁶, and R¹⁷ are independently selected from hydrogen and C₁₋₆alkyl; and wherein R⁵ is optionally substituted with one or more substituents independently selected from hydroxy, halo, C₁₋₄alkyl, C₁₋₄alkoxy, C₁₋₃haloalkyl, C₃₋₇cycloalkyl, cyano, and oxo;
R⁶ is hydrogen, fluoro, C₁₋₆alkyl, or C₃₋₇cycloalkyl; wherein when R⁶ is C₁₋₆alkyl or C₃₋₇cycloalkyl, said groups are optionally substituted with one or more substituents independently selected from hydroxy, halo, C₁₋₄alkyl, C₁₋₄alkoxy, C₁₋₃haloalkyl, and C₁₋₃haloalkoxy;
R⁷ is selected from hydrogen, deuterium, halo, and C₁₋₄alkyl; OR
R⁶ and R⁷, together with the carbon atom to which they are attached, form a 3- to 6-membered cycloalkyl or heterocyclyl ring, optionally substituted with one or more substituents independently selected from halo, C₁₋₄alkyl, C₁₋₄alkoxy, and C₁₋₃haloalkyl;
R⁹ is hydrogen or C₁₋₄alkyl;
R¹⁰ is hydroxy, C₁₋₄alkyl, or C₁₋₄alkoxy; and
R¹¹ and R¹² are independently selected from hydrogen, C₁₋₄alkyl, and C(O)OC₁₋₄alkyl.

Particular compounds of the disclosure include, for example, compounds of Formula I, or pharmaceutically acceptable salts thereof, wherein, unless otherwise stated, each of Z, X¹, X², X³, Y, R¹, R², R³, R⁴, R⁵, R⁶, R⁷, R¹⁰, R¹¹, and R¹² has any of the meanings defined hereinbefore, or in any of paragraphs (1) to (99) hereinafter. For the avoidance of doubt, the present disclosure encompasses combinations of two or more substituent definitions as described in paragraphs (1) to (99):
(1) Z is O;
(2) Z is CR⁶R⁷;
(3) One of X¹, X², and X³ is N, and the others are CR⁴;
(4) One of X¹ and X² is N, and X³ is CR⁴;
(5) X¹ is N, and X² and X³ are CR⁴;
(6) X¹ is N, and X² and X³ are CH;
(7) Two of X¹, X², and X³ are N, and the others are CR⁴;
(8) X¹ and X² are N, and X³ is CR⁴;
(9) X¹ and X³ are N, and X² is CR⁴;
(10) Y is selected from 5- or 6-membered monocyclic heteroaryl, 9- or 10-membered bicyclic heteroaryl, 5- or 6-membered heteroaryl fused to 5- or 6-membered cycloalkyl or 5- or 6-membered heterocyclyl, phenyl, heterocyclyl, C₃₋₇cycloalkyl, and QR⁸; wherein:
   i. Q is absent, O, or NR⁹;
   ii. R⁸ is C₁₋₆alkyl, C₁₋₃alkylene-phenyl, or C₁₋₃alkylene-heteroaryl;
   iii. Y is optionally substituted with one or more substituents Y^{A}, wherein at each occurrence Y^{A} is independently selected from halo, C₁₋₄alkyl, hydroxy, C₁₋₄alkoxy, C₁₋₃haloalkyl, cyano, C(O)R¹⁰, and C₃₋₇cycloalkyl; and
   iv. Y^{A} is optionally further substituted with one or more substituents independently selected from halo, C₁₋₄alkyl, hydroxy, C₁₋₄alkoxy, C₃₋₇cycloalkyl, and C₁₋₃alkylene-NR¹¹R¹²;
(11) Y is selected from 5- or 6-membered monocyclic heteroaryl, 9- or 10-membered bicyclic heteroaryl, 5- or 6-membered heteroaryl fused to 5- or 6-membered cycloalkyl or 5- or 6-membered heterocyclyl, phenyl, heterocyclyl, C₃₋₇cycloalkyl, and QR⁸; wherein:
   i. Q is absent, O, or NR⁹;
   ii. R⁸ is C₁₋₆alkyl, C₁₋₃alkylene-phenyl, or C₁₋₃alkylene-heteroaryl;
   iii. Y is optionally substituted with one or more substituents Y^{A}, wherein at each occurrence Y^{A} is independently selected from halo, C₁₋₄alkyl, C₁₋₃haloalkyl, cyano, C(O)R¹⁰, and C₃₋₇cycloalkyl; and
   iv. Y^{A} is optionally further substituted with one or more substituents independently selected from halo, hydroxy, C₃₋₇cycloalkyl, and C₁₋₃alkylene-NR¹¹R¹²;
(12) Y is selected from 5- or 6-membered monocyclic heteroaryl, 5- or 6-membered heteroaryl fused to 5- or 6-membered cycloalkyl or 5- or 6-membered heterocyclyl, heterocyclyl, cyclopropyl, and QR⁸; wherein:
   i. Q is absent, O, or NR⁹;
   ii. R⁸ is C₁₋₆alkyl, C₁₋₃alkylene-phenyl, or C₁₋₃alkylene-heteroaryl;
   iii. Y is optionally substituted with one or more substituents Y^{A}, wherein at each occurrence Y^{A} is independently selected from halo, C₁₋₄alkyl, C₁₋₃haloalkyl, cyano, C(O)R¹⁰, and C₃₋₇cycloalkyl; and
   iv. Y^{A} is optionally further substituted with one or more substituents independently selected from halo, hydroxy, C₃₋₇cycloalkyl, and C₁₋₃alkylene-NR¹¹R¹²;
(13) Y is selected from 5- or 6-membered monocyclic heteroaryl and QR⁸ wherein:
   i. Q is absent, O, or NR⁹;
   ii. R⁸ is C₁₋₆alkyl, C₁₋₃alkylene-phenyl, or C₁₋₃alkylene-heteroaryl;
   iii. Y is optionally substituted with one or more substituents Y^{A}, wherein at each occurrence Y^{A} is independently selected from halo, C₁₋₄alkyl, hydroxy, C₁₋₄alkoxy, C₁₋₂haloalkyl, cyano, C(O)R¹⁰, and C₃₋₇cycloalkyl; and
   iv. Y^{A} is optionally further substituted with one or more substituents independently selected from halo, C₁₋₄alkyl, hydroxy, C₁₋₄alkoxy, C₃₋₇cycloalkyl, and C₁₋₃alkylene-NR¹¹R¹²;
(14) Y is selected from 5- or 6-membered monocyclic heteroaryl and QR⁸ wherein:
   i. Q is absent, or O;
   ii. R⁸ is C₁₋₆alkyl, C₁₋₃alkylene-phenyl, or C₁₋₃alkylene-heteroaryl;
   iii. Y is optionally substituted with one or more substituents Y^{A}, wherein at each occurrence Y^{A} is independently selected from fluoro, chloro, C₁₋₄alkyl, hydroxy, C₁₋₄alkoxy, C₁₋₃haloalkyl, cyano, C(O)R¹⁰, and C₃₋₇cycloalkyl; and
   iv. Y^{A} is optionally further substituted with one or more substituents independently selected from halo, C₁₋₄alkyl, hydroxy, C₃₋₇cycloalkyl, and C₁₋₃alkylene-NR¹¹R¹²;
(15) Y is QR⁸ wherein Q is absent or O, R⁸ is C₁₋₆alkyl, CH₂-phenyl, CH₂-heteroaryl, CF₂-phenyl, or CF₂-heteroaryl, and said phenyl and heteroaryl groups are optionally substituted with one or more substituents Y^{A}, wherein at each occurrence Y^{A} is independently selected from fluoro, chloro, C₁₋₄alkyl, hydroxy, C₁₋₄alkoxy, C₁₋₃haloalkyl, cyano, C(O)R¹⁰, and cyclopropyl; and each Y^{A} is optionally further substituted with one or more substituents independently selected from halo, C₁₋₄alkyl, hydroxy, cyclopropyl, and C₁₋₃alkylene-NR¹¹R¹²;
(16) Y is QR⁸ wherein Q is absent or O, R⁸ is C₁₋₆alkyl, CH₂-phenyl, CH₂-heteroaryl, CF₂-phenyl, or CF₂-heteroaryl, and said phenyl and heteroaryl groups are optionally substituted with one or more substituents Y^{A}, wherein at each occurrence Y^{A} is independently selected from fluoro, chloro, C₁₋₄alkyl, C₁₋₃haloalkyl, cyano, C(O)R¹⁰, and cyclopropyl; and each Y^{A} is optionally further substituted with one or more substituents independently selected from halo, hydroxy, cyclopropyl, and C₁₋₃alkylene-NR¹¹R¹²;
(17) Y is OC₁₋₆alkyl, OCH₂-phenyl, OCH₂-heteroaryl, CH₂-phenyl, CH₂-heteroaryl, CF₂-phenyl, CF₂-heteroaryl, or NR⁹—CH₂-phenyl, wherein said phenyl and heteroaryl groups are optionally substituted with one or more substituents Y^{A}, wherein at each occurrence Y^{A} is independently selected from fluoro, chloro, C₁₋₄alkyl, C₁₋₃haloalkyl, cyano, C(O)R¹⁰, and cyclopropyl; and each Y^{A} is optionally further substituted with one or more substituents independently selected from halo, hydroxy, cyclopropyl, and C₁₋₃alkylene-NR¹¹R¹²;
(18) Y is O-tert-butyl, OCH₂-phenyl, OCH₂-heteroaryl, CH₂-heteroaryl, CF₂-phenyl, CF₂-heteroaryl, or NR⁹—CH₂-phenyl, wherein said phenyl and heteroaryl groups are optionally substituted with one or more substituents Y^{A}, wherein at each occurrence Y^{A} is independently selected from C₁₋₄alkyl (such as methyl) and C₁₋₃haloalkyl;
(19) Y is 5- or 6-membered monocyclic heteroaryl optionally substituted with one or more substituents Y^{A}, wherein at each occurrence Y^{A} is independently selected from halo, C₁₋₄alkyl, C₁₋₃haloalkyl, cyano, C(O)R¹⁰, and C₃₋₇cycloalkyl, and wherein each Y^{A} is optionally further substituted with one or more substituents independently selected from halo, C₁₋₄alkyl, hydroxy, C₁₋₄alkoxy, C₃₋₇cycloalkyl, C₁₋₃alkylene-NR¹¹R¹², and C₁₋₃haloalkyl;
(20) Y is 5- or 6-membered monocyclic heteroaryl optionally substituted with one or more substituents Y^{A}, wherein at each occurrence Y^{A} is independently selected from fluoro, chloro, C₁₋₄alkyl, C₁₋₃haloalkyl, cyano, C(O)R¹⁰, and cyclopropyl, and wherein each Y^{A} is optionally further substituted with one or more substituents independently selected from halo, hydroxy, cyclopropyl, and C₁₋₃alkylene-NR¹¹R¹²;
(21) Y is a 5- or 6-membered monocyclic heteroaryl selected from one of the following structures:

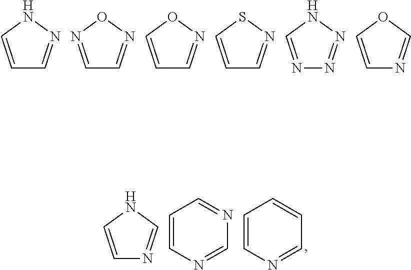

wherein the heteroaryl is optionally substituted with one or more substituents Y^{A} wherein at each occurrence Y^{A} is independently selected from fluoro, chloro, C₁₋₄alkyl, C₁₋₃haloalkyl, cyano, C(O)R¹⁰, and cyclopropyl, and wherein each Y^{A} is optionally further substituted with one or more substituents independently selected from halo, hydroxy, cyclopropyl, and C₁₋₃alkylene-NR¹¹R¹²;
(22) Y is a 5-membered monocyclic heteroaryl selected from one of the following structures:

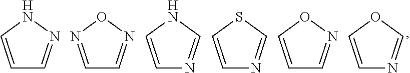

wherein the heteroaryl is optionally substituted with one or more substituents Y^{A} wherein at each occurrence Y^{A} is independently selected from C₁₋₄alkyl, cyano, and cyclopropyl, and wherein each Y^{A} is optionally further substituted with one or more substituents independently selected from fluoro, hydroxy, and C₁₋₃alkylene-NR¹¹R¹²;
(23) R¹ and R² are independently selected from hydrogen, C₁₋₄alkyl, C₃₋₇cycloalkyl, C₁₋₅alkoxy, and phenyl, wherein said C₁₋₄alkyl, C₃₋₇cycloalkyl, C₁₋₅alkoxy, or phenyl is optionally substituted with one or more substituents independently selected from fluoro, C₁₋₄alkyl, and trifluoromethyl; provided that R¹ and R² cannot be both hydrogen;
(24) R¹ and R² are independently selected from hydrogen, C₁₋₄alkyl, C₃₋₇cycloalkyl, and C₁₋₅alkoxy, wherein said C₁₋₄alkyl, C₃₋₇cycloalkyl, or C₁₋₅alkoxy is optionally substituted with one or more substituents independently selected from halo, C₁₋₄ alkyl, and C₁₋₃haloalkyl; provided that R¹ and R² cannot be both hydrogen;
(25) R¹ and R² are independently selected from C₁₋₄alkyl, C₃₋₇cycloalkyl, C₁₋₅alkoxy, and phenyl, wherein said C₁₋₄alkyl, C₃₋₇cycloalkyl, C₁₋₅alkoxy, or phenyl, is optionally substituted with one or more substituents independently selected from halo, C₁₋₄ alkyl, and C₁₋₃haloalkyl;
(26) R¹ and R² are independently selected from C₁₋₄alkyl, C₃₋₇cycloalkyl, and C₁₋₅alkoxy, wherein said C₁₋₄alkyl, C₃₋₇cycloalkyl, or C₁₋₅alkoxy is optionally substituted with one or more substituents independently selected from halo and C₁₋₄alkyl;
(27) R¹ and R² are independently selected from C₁₋₄alkyl and C₁₋₅alkoxy, each optionally substituted with one or more halo substituents;
(28) One of R¹ and R² is C₁₋₄alkyl, and the other is C₁₋₅alkoxy, each optionally substituted with one or more halo substituents;
(29) One of R¹ and R² is C₁₋₂alkyl, and the other is C₂₋₅alkoxy, each optionally substituted with one or more halo substituents;
(30) One of R¹ and R² is methyl, and the other is C₁₋₅alkoxy, each optionally substituted with one or more halo substituents;
(31) One of R¹ and R² is C₁₋₄alkyl, and the other is tert-butoxy, each optionally substituted with one or more halo substituents;
(32) One of R¹ and R² is methyl, and the other is tert-butoxy, each optionally substituted with one or more halo substituents;
(33) One of R¹ and R² is methyl, and the other is tert-butoxy;
(34) One of R¹ and R² is hydrogen, and the other is C₁₋₅alkoxy;
(35) One of R¹ and R² is hydrogen, and the other is tert-butoxy;
(36) R¹ and R² are independently selected C₃₋₇cycloalkyl rings, each optionally substituted with one or more substituents independently selected from halo and C₁₋₄ alkyl;
(37) R¹ and R² are independently selected C₃₋₅cycloalkyl rings, each optionally substituted with one or more substituents independently selected from halo and C₁₋₄ alkyl;
(38) R¹ and R² are both cyclopropyl rings, each ring being independently optionally substituted with one or more substituents independently selected from halo and C₁₋₄ alkyl (such as fluoro and methyl);
(39) R¹ and R² are both cyclopropyl;
(40) R¹ and R² together with the carbon atom to which they are attached form a 4- to 8-membered cycloalkyl ring, wherein the cycloalkyl ring is optionally substituted with one or more substituents independently selected from halo, C₁₋₄alkyl, and C₁₋₃haloalkyl;
(41) R¹ and R² together with the carbon atom to which they are attached form a 4- to 8-membered cycloalkyl ring, wherein the cycloalkyl ring is optionally substituted with one or more substituents independently selected from fluoro, methyl, and trifluoromethyl;
(42) R¹ and R² together with the carbon atom to which they are attached form a 5- to 8-membered cycloalkyl ring, wherein the cycloalkyl ring is optionally substituted with one or more substituents independently selected from halo, and C₁₋₄alkyl;
(43) R¹ and R² together with the carbon atom to which they are attached form a 6- to 8-membered cycloalkyl ring, wherein the cycloalkyl ring is optionally substituted with one or more substituents independently selected from fluoro, chloro, methyl, and trifluoromethyl;
(44) R¹ and R² together with the carbon atom to which they are attached form a 6- to 8-membered cycloalkyl ring, wherein the cycloalkyl ring is optionally substituted with one or more substituents independently selected from fluoro, methyl, and trifluoromethyl;
(45) R¹ and R² together with the carbon atom to which they are attached form a cyclohexyl ring, wherein the cyclohexyl ring is substituted with one or more substituents independently selected from halo, C₁₋₄alkyl, and C₁₋₃haloalkyl;
(46) R¹ and R² together with the carbon atom to which they are attached form a cyclohexyl ring, wherein the cyclohexyl ring is substituted with one or more substituents independently selected from halo and C₁₋₄alkyl;
(47) R¹ and R² together with the carbon atom to which they are attached form a cyclohexyl ring, wherein the cyclohexyl ring is substituted with one or more substituents independently selected from fluoro and C₁₋₄alkyl;
(48) R¹ and R² together with the carbon atom to which they are attached form a cyclohexyl ring, wherein the cyclohexyl ring is substituted with one or more substituents independently selected from halo and methyl;
(49) R¹ and R² together with the carbon atom to which they are attached form a cyclohexyl ring, wherein the cyclohexyl ring is substituted with one or more substituents independently selected from fluoro and methyl;
(50) R¹ and R² together with the carbon atom to which they are attached form a cyclohexyl ring, wherein the cyclohexyl ring is substituted with a methyl substituent;
(51) R¹ and R² together with the carbon atom to which they are attached form a cyclohexyl ring, wherein the cyclohexyl ring is substituted with one or more fluoro substituents;
(52) R¹ and R² together with the carbon atom to which they are attached form a cyclohexyl ring, wherein the cyclohexyl ring is substituted with two fluoro substituents;
(53) The combined R¹, R², and R³ group is a group selected from:

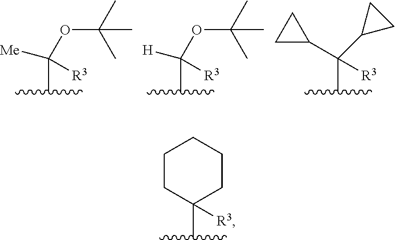

wherein [symbol] is the point of attachment to the rest of the compound of Formula I; [symbol] and each cyclopropyl or cyclohexyl ring is optionally substituted with one or more substituents independently selected from halo, C₁₋₄alkyl, and C₁₋₃haloalkyl;
(54) The combined R¹, R², and R³ group is a group selected from:

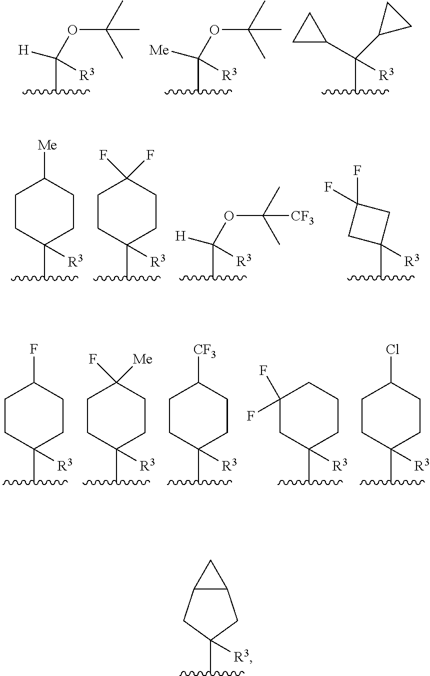

wherein [symbol] is the point of attachment to the rest of the compound of Formula I;
(55) The combined R¹, R², and R³ group is a group selected from:

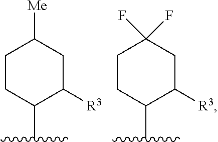

wherein [symbol] is the point of attachment to the rest of the compound of Formula I;
(56) R³ is hydrogen or methyl;
(57) R³ is hydrogen;
(58) R³ is methyl;
(59) R⁴ at each occurrence is independently selected from hydrogen, fluoro, methyl, and hydroxy;
(60) R⁴ at each occurrence is independently selected from hydrogen, fluoro, and methyl;
(61) R⁴ at each occurrence is independently selected from hydrogen and fluoro;
(62) R⁴ at each occurrence is hydrogen;
(63) R⁴ is hydrogen, X¹ is nitrogen, and X² and X³ are CH;
(64) R⁴ is hydrogen, X¹ is nitrogen, X² is CH, and X³ is CF;
(65) R⁵ is selected from 5- or 6-membered monocyclic heteroaryl, 4- to 10-membered heterocyclyl, C(O)NR¹³R¹⁴, OC(O)NR¹⁵R¹⁶, and NR¹⁷C(O)C₁₋₆alkyl, wherein R¹³, R¹⁴, R¹⁵, R¹⁶, and R¹⁷ are independently selected from hydrogen and C₁₋₆alkyl; and wherein R⁵ is optionally substituted with one or more substituents independently selected from halo, C₁₋₄alkyl, C₁₋₃haloalkyl, C₃₋₇cycloalkyl, and oxo;
(66) R⁵ is selected from 5- or 6-membered monocyclic heteroaryl, 4- to 10-membered heterocyclyl, C(O)NR¹³R¹⁴, OC(O)NR¹⁵R¹⁶, and NR¹⁷C(O)C₁₋₆alkyl, wherein R¹³, R¹⁴, R¹⁵, R¹⁶, and R¹⁷ are independently selected from hydrogen and C₁₋₆alkyl; and wherein R⁵ is optionally substituted with one or more substituents independently selected from fluoro, chloro, methyl, C₁₋₂haloalkyl, and oxo;
(67) R⁵ is selected from 5- or 6-membered monocyclic heteroaryl, 5- to 8-membered heterocyclyl, C(O)NR¹³R¹⁴, OC(O)NR¹⁵R¹⁶, and NR¹⁷C(O)C₁₋₆alkyl, wherein R¹³, R¹⁴, R¹⁵, R¹⁶, and R¹⁷ are independently selected from hydrogen and C₁₋₆alkyl; and wherein R⁵ is optionally substituted with one or more substituents independently selected from fluoro, methyl, trifluoromethyl, and oxo;
(68) R⁵ is selected from 5-membered monocyclic heteroaryl, 5-membered heterocyclyl, and NR¹⁷C(O)C₁₋₆alkyl, wherein R¹⁷ is hydrogen or methyl; and wherein R⁵ is optionally substituted with one or more substituents independently selected from hydroxy, halo, C₁₋₄alkyl, C₁₋₄alkoxy, C₁₋₃haloalkyl, C₃₋₇cycloalkyl, cyano, and oxo;
(69) R⁵ is selected from 5-membered monocyclic heteroaryl, 5-membered heterocyclyl, and NR¹⁷C(O)C₁₋₆alkyl, wherein R¹⁷ is hydrogen or methyl; and wherein R⁵ is optionally substituted with one or more substituents independently selected from fluoro, methyl, trifluoromethyl, and oxo;
(70) R⁵ has the structure:

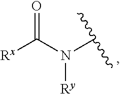

wherein [symbol] is the point of attachment to the rest of the compound of Formula I; R^{x} is C₁₋₆alkyl optionally substituted with one or more substituents independently selected from hydroxy, halo, C₁₋₄alkoxy, C₁₋₃haloalkyl, and C₃₋₇cycloalkyl; and R^{y} is hydrogen or C₁₋₆alkyl; or R^{x} and R^{y}, taken together with the atoms to which they are attached, form a 5- to 8-membered heterocyclic ring, optionally substituted with one or more substituents independently selected from hydroxy, halo, C₁₋₄alkyl, C₁₋₄alkoxy, C₁₋₇haloalkyl, C₃₋₇cycloalkyl, and oxo;
(71) R⁵ has the structure:

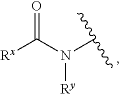

wherein [symbol] is the point of attachment to the rest of the compound of Formula I; R^{x} is C₁₋₄alkyl optionally substituted with one or more substituents independently selected from hydroxy, fluoro, methoxy, and cyclopropyl; and R^{y} is hydrogen or methyl; or R^{x} and R^{y}, taken together with the atoms to which they are attached, form a 5-membered heterocyclic ring, optionally substituted with one or more substituents independently selected from fluoro, C₁₋₄alkyl, trifluoromethyl, and oxo;
(72) R⁵ has a structure selected from:

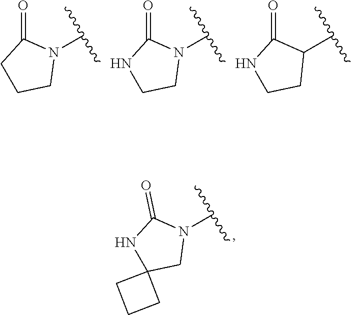

wherein [symbol] is the point of attachment to the rest of the compound of Formula I; and each structure is optionally substituted with one or more substituents independently selected from fluoro, C₁₋₄alkyl (e.g. methyl), trifluoromethyl, and oxo;
(73) R⁵ has the structure:

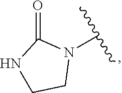

wherein [symbol] is the point of attachment to the rest of the compound of Formula I; and the structure is optionally substituted with one or more substituents independently selected from fluoro, methyl, trifluoromethyl, and oxo;
(74) R⁵ has a structure selected from:

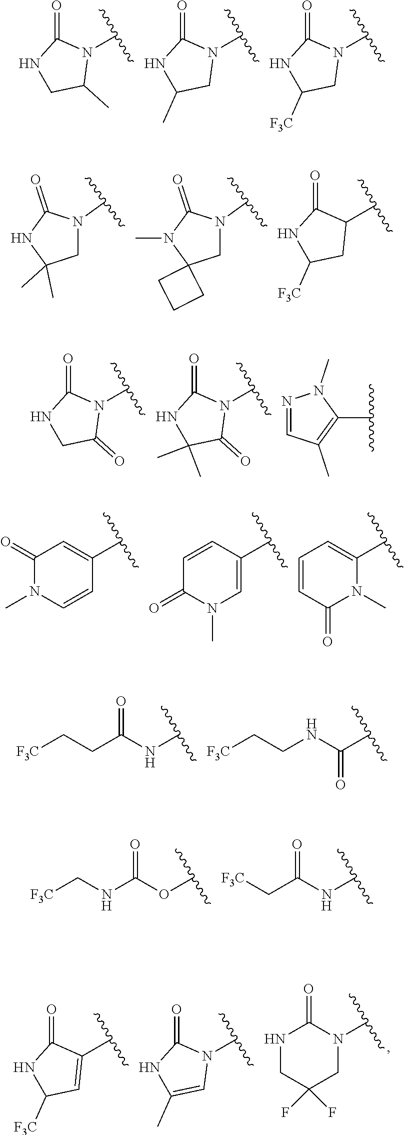

wherein [symbol] is the point of attachment to the rest of the compound of Formula I;
(75) R⁵ has a structure selected from:

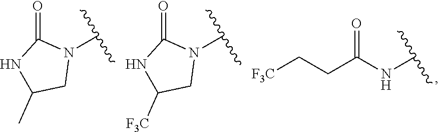

wherein [symbol] is the point of attachment to the rest of the compound of Formula I;
(76) R⁶ is hydrogen, C₁₋₆alkyl C₃₋₇cycloalkyl; wherein when R⁶ is C₁₋₆alkyl or C₃₋₇cycloalkyl, said groups are optionally substituted with one or more substituents independently selected from hydroxy, halo, C₁₋₄alkyl, C₁₋₄alkoxy, C₁₋₃haloalkyl, and C₁₋₃haloalkoxy;
(77) R⁶ is hydrogen, C₁₋₆alkyl, or C₃₋₇cycloalkyl; wherein when R⁶ is C₁₋₆alkyl or C₃₋₇cycloalkyl, said groups are optionally substituted with one or more substituents independently selected from hydroxy, halo, C₁₋₄alkyl, C₁₋₄alkoxy, and C₁₋₃haloalkyl;
(78) R⁶ is hydrogen, C₁₋₃alkyl, or C₃₋₅cycloalkyl; wherein when R⁶ is C₁₋₃alkyl or C₃₋₅cycloalkyl, said groups are optionally substituted with one or more substituents independently selected from hydroxy, fluoro, methyl, C₁₋₂alkoxy, and C₁₋₂haloalkyl;
(79) R⁶ is hydrogen, methyl, or cyclopropyl; wherein when R⁶ is methyl or cyclopropyl, said groups are optionally substituted with one or more substituents independently selected from fluoro and methoxy;
(80) R⁶ is hydrogen;
(81) R⁶ is cyclopropyl;
(82) R⁶ is methyl optionally substituted with one or more substituents independently selected from fluoro, hydroxy, and methoxy;
(83) R⁶ is CH₂OMe;
(84) R⁷ is selected from hydrogen, deuterium, fluoro, and methyl;
(85) R⁷ is selected from hydrogen and deuterium;
(86) R⁷ is hydrogen;
(87) R⁶ and R⁷, together with the carbon atom to which they are attached, form a 4- to 6-membered cycloalkyl or heterocyclyl ring, optionally substituted with one or more substituents independently selected from halo, C₁₋₄alkyl, C₁₋₄alkoxy, and C₁₋₃haloalkyl;
(88) R⁶ and R⁷, together with the carbon atom to which they are attached, form a 5- or 6-membered cycloalkyl or heterocyclyl ring, optionally substituted with one or more substituents independently selected from fluoro, methyl, methoxy, and trifluoromethyl;
(89) R⁶ and R⁷, together with the carbon atom to which they are attached, form a 4- to 6-membered cycloalkyl ring, optionally substituted with one or more substituents independently selected from halo, C₁₋₄alkyl, C₁₋₄alkoxy, and C₁₋₃haloalkyl;
(90) R⁶ and R⁷, together with the carbon atom to which they are attached, form a 5- or 6-membered cycloalkyl ring, optionally substituted with one or more substituents independently selected from fluoro, methyl, methoxy, and trifluoromethyl;
(91) R¹⁰ is hydroxy, or C₁₋₄alkoxy;
(92) R¹⁰ is hydroxy or C₃₋₄alkoxy;
(93) R¹⁰ is C₁₋₄alkoxy;
(94) R¹⁰ is tert-butoxy;
(95) R¹¹ and R¹² are independently selected from hydrogen, methyl, and C(O)OC₁₋₄alkyl;
(96) R¹¹ and R¹² are independently selected from hydrogen and C(O)OC₁₋₄alkyl;
(97) R¹¹ and R¹² are both hydrogen;
(98) R¹¹ is hydrogen, and R¹² is C(O)OC₁₋₄alkyl;
(99) R¹¹ is hydrogen, and R¹² is C(O)Otert-butyl.

In one embodiment, Z is as defined in any one of paragraphs (1) to (2) above. In a further embodiment, Z is as defined in paragraph (2) above.

In one embodiment, X¹ to X³ are as defined in any one of paragraphs (3) to (9) above. In a further embodiment, X¹ to X³ are as defined in paragraph (5) above. In a further embodiment, X¹ to X³ are as defined in paragraph (6) above.

In one embodiment, Y is as defined in any one of paragraphs (10) to (22) above. In a further embodiment, Y is as defined in paragraphs (13) to (22) above. In a further embodiment, Y is as defined in paragraph (18) above. In a further embodiment, Y is as defined in paragraph (22) above.

In one embodiment, R¹ and R² are as defined in any one of paragraphs (23) to (55) above. In a further embodiment, R¹ and R² are as defined in paragraphs (53) to (55) above. In a further embodiment, R¹ and R² are as defined in paragraph (54) above. In a further embodiment, R¹ and R² are as defined in paragraph (55) above.

In one embodiment, R³ is as defined in any one of paragraphs (56) to (58) above. In a further embodiment, R³ is as defined in paragraph (57) above.

In one embodiment, R⁴ is as defined in any one of paragraphs (59) to (64) above. In a further embodiment, R⁴ is as defined in paragraph (61) or (62) above.

In one embodiment, R⁵ is as defined in any one of paragraphs (65) to (75) above. In a further embodiment, R⁵ is as defined in paragraph (74) or (75) above.

In one embodiment, R⁶ is as defined in any one of paragraphs (76) to (83) above. In a further embodiment, R⁶ is as defined in any one of paragraphs (80) to (83) above.

In one embodiment, R⁷ is as defined in any one of paragraphs (84) to (86) above. In a further embodiment, R⁷ is as defined in paragraph (86) above.

In one embodiment, R⁶ and R⁷ are as defined in any one of paragraphs (87) to (90) above. In a further embodiment, R⁶ and R⁷ are as defined in paragraph (90) above.

In one embodiment, R¹⁰ is as defined in any one of paragraphs (91) to (94) above.

In a further embodiment, R¹⁰ is as defined in paragraph (94) above.

In one embodiment, R¹¹ and R¹² are as defined in any one of paragraphs (95) to (99) above. In a further embodiment, R¹¹ and R¹² are as defined in paragraph (97) or (99) above.

In an embodiment, there is provided a compound according to any one of formula IA to IF (sub-formulae of formula I), or a pharmaceutically acceptable salt thereof:

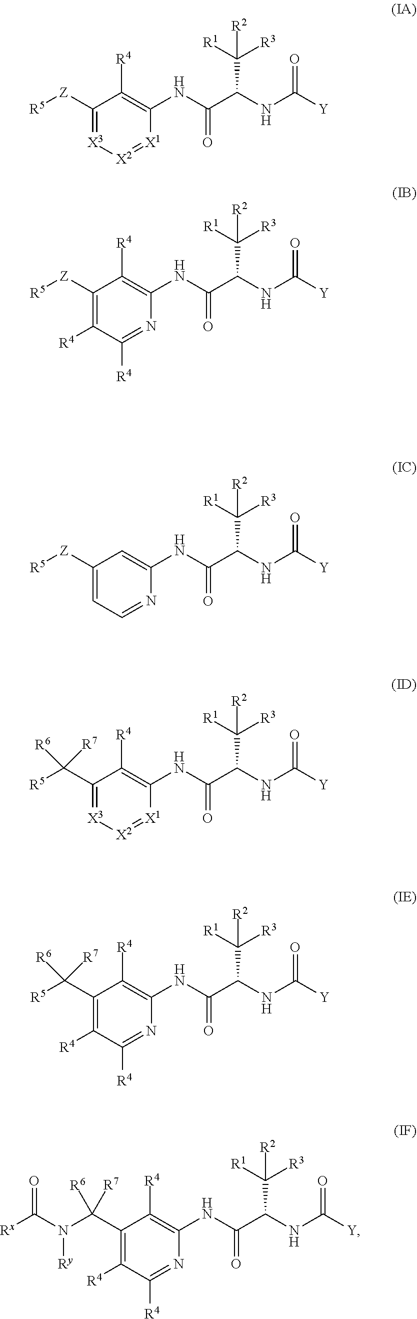

wherein Z, X¹, X², X³, Y, R¹, R², R³, R⁴, R⁵, R⁶, and R⁷ are as described hereinabove; R^{x} is C₁₋₆alkyl optionally substituted with one or more substituents independently selected from hydroxy, halo, C₁₋₄alkoxy, C₁₋₃haloalkyl, and C₃₋₇cycloalkyl, and R^{y} is hydrogen or C₁₋₆alkyl; or R^{x} and R^{y}, taken together with the atoms to which they are attached, form a 5- to 8-membered heterocyclic ring, optionally substituted with one or more substituents independently selected from hydroxy, halo, C₁₋₄alkyl, C₁₋₄alkoxy, C₁₋₃haloalkyl, C₃₋₇cycloalkyl, and oxo.

In an embodiment, there is provided a compound according to any one of formula IA to IF, or a pharmaceutically acceptable salt thereof, wherein Z is as defined in paragraph (2) above; X¹ to X³ are as defined in paragraph (5) or (6) above; Y is as defined in paragraph (18) or (22) above; R¹ and R² are as defined in paragraphs (53) to (55) above;
R³ is as defined in paragraph (57) above; R⁴ is as defined in paragraph (61) or (62) above;
R⁵ is as defined in paragraph (74) or (75) above; R⁶ is as defined in any one of paragraphs (80) to (83) above; and/or R⁷ is as defined in paragraph (86) above; and R^{x} is C₁₋₄alkyl optionally substituted with one or more substituents independently selected from hydroxy, fluoro, methoxy, and cyclopropyl; and R^{y} is hydrogen or methyl; or R^{x} and R^{y}, taken together with the atoms to which they are attached, form a 5-membered heterocyclic ring, optionally substituted with one or more substituents independently selected from fluoro, C₁₋₄alkyl, trifluoromethyl, and oxo.

Particular compounds of the present disclosure include any one of the following compounds, or a pharmaceutically acceptable salt thereof:
Tert-butyl ((S)-1-((1r,4S)-4-methylcyclohexyl)-2-oxo-2-((4-(((S)-2-oxo-4-(trifluoromethyl)imidazolidin-1-yl)methyl)pyridin-2-yl)amino)ethyl)carbamate;
4-Cyclopropyl-N—((S)-1-((1r,4S)-4-methylcyclohexyl)-2-oxo-2-((4-(((S)-2-oxo-4-(trifluoromethyl)imidazolidin-1-yl)methyl)pyridin-2-yl)amino)ethyl)-1,2,5-oxadiazole-3-carboxamide;
1-Methyl-N—((S)-1-((1r,4S)-4-methylcyclohexyl)-2-oxo-2-((4-(((S)-2-oxo-4-(trifluoromethyl)imidazolidin-1-yl)methyl)pyridin-2-yl)amino)ethyl)-1H-pyrazole-5-carboxamide;
1-Isopropyl-N—((S)-1-((1r,4S)-4-methylcyclohexyl)-2-oxo-2-((4-(((S)-2-oxo-4-(trifluoromethyl)imidazolidin-1-yl)methyl)pyridin-2-yl)amino)ethyl)-1H-pyrazole-5-carboxamide;
Benzyl ((S)-1-((1r,4S)-4-methylcyclohexyl)-2-oxo-2-((4-(((S)-2-oxo-4-(trifluoromethyl)imidazolidin-1-yl)methyl)pyridin-2-yl)amino)ethyl)carbamate;
Tert-butyl ((S)-1-(4,4-difluorocyclohexyl)-2-oxo-2-((4-(((S)-2-oxo-4-(trifluoromethyl)imidazolidin-1-yl)methyl)pyridin-2-yl)amino)ethyl)carbamate;
Tert-butyl 6-(((S)-1-((1r,4S)-4-methylcyclohexyl)-2-oxo-2-((4-(((S)-2-oxo-4-(trifluoromethyl)imidazolidin-1-yl)methyl)pyridin-2-yl)amino)ethyl)carbamoyl)-3,4-dihydropyrrolo[1,2-a]pyrazine-2(1H)-carboxylate;
4-Cyclopropyl-N—((S)-1-(4,4-difluorocyclohexyl)-2-oxo-2-((4-(((S)-2-oxo-4-(trifluoromethyl)imidazolidin-1-yl)methyl)pyridin-2-yl)amino)ethyl)-1,2,5-oxadiazole-3-carboxamide;
N—((S)-1-(4,4-Difluorocyclohexyl)-2-oxo-2-((4-(((S)-2-oxo-4-(trifluoromethyl)imidazolidin-1-yl)methyl)pyridin-2-yl)amino)ethyl)-1-methyl-1H-pyrazole-5-carboxamide;
Benzyl ((S)-1-(4,4-difluorocyclohexyl)-2-oxo-2-((4-(((S)-2-oxo-4-(trifluoromethyl)imidazolidin-1-yl)methyl)pyridin-2-yl)amino)ethyl)carbamate;
Tert-butyl 6-(((S)-1-(4,4-difluorocyclohexyl)-2-oxo-2-((4-(((S)-2-oxo-4-(trifluoromethyl)imidazolidin-1-yl)methyl)pyridin-2-yl)amino)ethyl)carbamoyl)-3,4-dihydropyrrolo[1,2-a]pyrazine-2(1H)-carboxylate;
N—((S)-1-(4,4-Difluorocyclohexyl)-2-oxo-2-((4-(((S)-2-oxo-4-(trifluoromethyl)imidazolidin-1-yl)methyl)pyridin-2-yl)amino)ethyl)-1-isopropyl-1H-pyrazole-5-carboxamide;
N—((S)-1-(4,4-Difluorocyclohexyl)-2-oxo-2-((4-(((S)-2-oxo-4-(trifluoromethyl)imidazolidin-1-yl)methyl)pyridin-2-yl)amino)ethyl)-3-ethylisoxazole-4-carboxamide;
1-Cyclopropyl-N—((S)-1-(4,4-difluorocyclohexyl)-2-oxo-2-((4-(((S)-2-oxo-4-(trifluoromethyl)imidazolidin-1-yl)methyl)pyridin-2-yl)amino)ethyl)-1H-pyrazole-5-carboxamide;
N—((S)-1,1-Dicyclopropyl-3-oxo-3-((4-(((S)-2-oxo-4-(trifluoromethyl)imidazolidin-1-yl)methyl)pyridin-2-yl)amino)propan-2-yl)-1-methyl-1H-pyrazole-5-carboxamide;
4-Cyclopropyl-N—((S)-1,1-dicyclopropyl-3-oxo-3-((4-(((S)-2-oxo-4-(trifluoromethyl)imidazolidin-1-yl)methyl)pyridin-2-yl)amino)propan-2-yl)-1,2,5-oxadiazole-3-carboxamide;
N—((S)-1,1-Dicyclopropyl-3-oxo-3-((4-(((S)-2-oxo-4-(trifluoromethyl)imidazolidin-1-yl)methyl)pyridin-2-yl)amino)propan-2-yl)-1-isopropyl-1H-pyrazole-5-carboxamide;
N—((S)-1,1-Dicyclopropyl-3-oxo-3-((4-(((S)-2-oxo-4-(trifluoromethyl)imidazolidin-1-yl)methyl)pyridin-2-yl)amino)propan-2-yl)-4-methyl-1,2,5-oxadiazole-3-carboxamide;
Tert-butyl ((1-(4-(((S)-1-(4,4-difluorocyclohexyl)-2-oxo-2-((4-(((S)-2-oxo-4-(trifluoromethyl)imidazolidin-1-yl)methyl)pyridin-2-yl)amino)ethyl)carbamoyl)isoxazol-3-yl)cyclopropyl)methyl)carbamate;
3-Cyclopropyl-N—((S)-1-(4,4-difluorocyclohexyl)-2-oxo-2-((4-(((S)-2-oxo-4-(trifluoromethyl)imidazolidin-1-yl)methyl)pyridin-2-yl)amino)ethyl)isoxazole-4-carboxamide;
N—((S)-1-(4,4-Difluorocyclohexyl)-2-oxo-2-((4-(((S)-2-oxo-4-(trifluoromethyl)imidazolidin-1-yl)methyl)pyridin-2-yl)amino)ethyl)-3-isopropylisoxazole-4-carboxamide;
3-(1-(Aminomethyl)cyclopropyl)-N—((S)-1-(4,4-difluorocyclohexyl)-2-oxo-2-((4-(((S)-2-oxo-4-(trifluoromethyl)imidazolidin-1-yl)methyl)pyridin-2-yl)amino)ethyl)isoxazole-4-carboxamide;
Tert-butyl ((S)-2-((4-((5-methyl-6-oxo-5,7-diazaspiro[3.4]octan-7-yl)methyl)pyridin-2-yl)amino)-1-((1r,4S)-4-methylcyclohexyl)-2-oxoethyl)carbamate;
Tert-butyl ((S)-2-((4-((4,4-dimethyl-2-oxoimidazolidin-1-yl)methyl)pyridin-2-yl)amino)-1-((1r,4S)-4-methylcyclohexyl)-2-oxoethyl)carbamate;
4-Cyclopropyl-N—((S)-2-((4-((4,4-dimethyl-2-oxoimidazolidin-1-yl)methyl)pyridin-2-yl)amino)-1-((1r,4S)-4-methylcyclohexyl)-2-oxoethyl)-1,2,5-oxadiazole-3-carboxamide;
Tert-butyl ((S)-1-((1r,4S)-4-methylcyclohexyl)-2-oxo-2-((4-((((2,2,2-trifluoroethyl)carbamoyl)oxy)methyl)pyridin-2-yl)amino)ethyl)carbamate;
Tert-butyl (S)-(1-(4,4-difluorocyclohexyl)-2-((4-((4,4-dimethyl-2,5-dioxoimidazolidin-1-yl)methyl)pyridin-2-yl)amino)-2-oxoethyl)carbamate;
Tert-butyl (S)-(1-(4,4-difluorocyclohexyl)-2-((4-((2,5-dioxoimidazolidin-1-yl)methyl)pyridin-2-yl)amino)-2-oxoethyl)carbamate;
Tert-butyl ((S)-2-((4-(((R)-5-methyl-2-oxoimidazolidin-1-yl)methyl)pyridin-2-yl)amino)-1-((1r,4S)-4-methylcyclohexyl)-2-oxoethyl)carbamate;
Tert-butyl ((S)-2-((4-(((R)-4-methyl-2-oxoimidazolidin-1-yl)methyl)pyridin-2-yl)amino)-1-((1r,4S)-4-methylcyclohexyl)-2-oxoethyl)carbamate;
(S)—N-(1,1-Dicyclopropyl-3-oxo-3-((4-(2-oxo-2-((3,3,3-trifluoropropyl)amino)ethyl)pyridin-2-yl)amino)propan-2-yl)-1-methyl-1H-pyrazole-5-carboxamide;
(S)—N-(1,1-Dicyclopropyl-3-oxo-3-((4-((4,4,4-trifluorobutanamido)methyl)pyridin-2-yl)amino)propan-2-yl)-1-methyl-1H-pyrazole-5-carboxamide;
N—((S)-1,1-Dicyclopropyl-3-((4-(((R)-4-methyl-2-oxoimidazolidin-1-yl)methyl)pyridin-2-yl)amino)-3-oxopropan-2-yl)-1-methyl-1H-pyrazole-5-carboxamide;
1-Cyclopropyl-N—((S)-1,1-dicyclopropyl-3-((4-(((R)-4-methyl-2-oxoimidazolidin-1-yl)methyl)pyridin-2-yl)amino)-3-oxopropan-2-yl)-1H-pyrazole-5-carboxamide;
N-(1-(4,4-Difluorocyclohexyl)-2-((4-((1-methyl-6-oxo-1,6-dihydropyridin-2-yl)methyl)pyridin-2-yl)amino)-2-oxoethyl)-1-isopropyl-1H-pyrazole-5-carboxamide;
N-(1-(4,4-Difluorocyclohexyl)-2-((4-((1-methyl-6-oxo-1,6-dihydropyridin-2-yl)methyl)pyridin-2-yl)amino)-2-oxoethyl)-1-ethyl-1H-pyrazole-5-carboxamide;
N-(1-(4,4-Difluorocyclohexyl)-2-((4-((1-methyl-6-oxo-1,6-dihydropyridin-2-yl)methyl)pyridin-2-yl)amino)-2-oxoethyl)-1-methyl-1H-pyrazole-5-carboxamide;
N-((2S)-1,1-Dicyclopropyl-3-((4-(cyclopropyl(4,4,4-trifluorobutanamido)methyl)pyridin-2-yl)amino)-3-oxopropan-2-yl)-1-methyl-1H-pyrazole-5-carboxamide;
4-Cyclopropyl-N-((2S)-1,1-dicyclopropyl-3-((4-(cyclopropyl(4,4,4-trifluorobutanamido)methyl)pyridin-2-yl)amino)-3-oxopropan-2-yl)-1,2,5-oxadiazole-3-carboxamide;
N-((2S)-1,1-Dicyclopropyl-3-((4-(cyclopropyl(4,4,4-trifluorobutanamido)methyl)pyridin-2-yl)amino)-3-oxopropan-2-yl)-1-isopropyl-1H-pyrazole-5-carboxamide;
N-((2S)-1,1-Dicyclopropyl-3-((4-(cyclopropyl(4,4,4-trifluorobutanamido)methyl)pyridin-2-yl)amino)-3-oxopropan-2-yl)-3-ethylisoxazole-4-carboxamide;
Tert-butyl (S)-(1-(4,4-difluorocyclohexyl)-2-oxo-2-((4-((((2,2,2-trifluoroethyl)carbamoyl)oxy)methyl)pyridin-2-yl)amino)ethyl)carbamate;
4-Cyclopropyl-N-(2-((4-(cyclopropyl(4,4,4-trifluorobutanamido)methyl)pyridin-2-yl)amino)-1-(4,4-difluorocyclohexyl)-2-oxoethyl)-1,2,5-oxadiazole-3-carboxamide;
N-(2-((4-(Cyclopropyl(4,4,4-trifluorobutanamido)methyl)pyridin-2-yl)amino)-1-(4,4-difluorocyclohexyl)-2-oxoethyl)-1-methyl-1H-pyrazole-5-carboxamide;
N-(2-((4-(Cyclopropyl(4,4,4-trifluorobutanamido)methyl)pyridin-2-yl)amino)-1-(4,4-difluorocyclohexyl)-2-oxoethyl)-1-isopropyl-1H-pyrazole-5-carboxamide;
N-(2-((4-(Cyclopropyl(4,4,4-trifluorobutanamido)methyl)pyridin-2-yl)amino)-1-(4,4-difluorocyclohexyl)-2-oxoethyl)-3-ethylisoxazole-4-carboxamide;
Tert-butyl ((S)-2-((5-fluoro-4-(((S)-2-oxo-4-(trifluoromethyl)imidazolidin-1-yl)methyl)pyridin-2-yl)amino)-1-((1r,4S)-4-methylcyclohexyl)-2-oxoethyl)carbamate;
Tert-butyl ((S)-2-((4-((1,4-dimethyl-1H-pyrazol-5-yl)methyl)pyridin-2-yl)amino)-1-((1r,4S)-4-methylcyclohexyl)-2-oxoethyl)carbamate;
N-(1-(4,4-Difluorocyclohexyl)-2-((4-((1-methyl-6-oxo-1,6-dihydropyridin-3-yl)methyl)pyridin-2-yl)amino)-2-oxoethyl)-1-methyl-1H-pyrazole-5-carboxamide;
N-(1-(4,4-Difluorocyclohexyl)-2-((4-((1,4-dimethyl-1H-pyrazol-5-yl)methyl)pyridin-2-yl)amino)-2-oxoethyl)-1-methyl-1H-pyrazole-5-carboxamide;
N-(1-(4,4-Difluorocyclohexyl)-2-((4-((1-methyl-2-oxo-1,2-dihydropyridin-4-yl)methyl)pyridin-2-yl)amino)-2-oxoethyl)-1-methyl-1H-pyrazole-5-carboxamide;
N—((S)-2-((4-((1,4-Dimethyl-1H-pyrazol-5-yl)methyl)pyridin-2-yl)amino)-1-((1r,4S)-4-methylcyclohexyl)-2-oxoethyl)-1-isopropyl-1H-pyrazole-5-carboxamide;
(1R,2S,5S)—N-((2S)-1,1-Dicyclopropyl-3-((4-(cyclopropyl(4,4,4-trifluorobutanamido)methyl)pyridin-2-yl)amino)-3-oxopropan-2-yl)-3,6,6-trimethyl-3-azabicyclo[3.1.0]hexane-2-carboxamide;
(5-Methylisoxazol-3-yl)methyl ((S)-1-(4,4-difluorocyclohexyl)-2-oxo-2-((4-(((S)-2-oxo-4-(trifluoromethyl)imidazolidin-1-yl)methyl)pyridin-2-yl)amino)ethyl)carbamate;
4-Cyclopropyl-N-((1S)-2-((4-(2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)-1,2,5-oxadiazole-3-carboxamide;
(1,5-Dimethyl-1H-pyrazol-3-yl)methyl ((S)-1-(4,4-difluorocyclohexyl)-2-oxo-2-((4-(((S)-2-oxo-4-(trifluoromethyl)imidazolidin-1-yl)methyl)pyridin-2-yl)amino)ethyl)carbamate;
4-Cyclopropyl-N—((S)-2-((5-fluoro-4-(((S)-2-oxo-4-(trifluoromethyl)imidazolidin-1-yl)methyl)pyridin-2-yl)amino)-1-((1r,4S)-4-methylcyclohexyl)-2-oxoethyl)-1,2,5-oxadiazole-3-carboxamide;
N—((S)-2-((5-Fluoro-4-(((S)-2-oxo-4-(trifluoromethyl)imidazolidin-1-yl)methyl)pyridin-2-yl)amino)-1-((1r,4S)-4-methylcyclohexyl)-2-oxoethyl)-4-methyl-1,2,5-oxadiazole-3-carboxamide;
N—((S)-2-((5-Fluoro-4-(((S)-2-oxo-4-(trifluoromethyl)imidazolidin-1-yl)methyl)pyridin-2-yl)amino)-1-((1r,4S)-4-methylcyclohexyl)-2-oxoethyl)-1-isopropyl-1H-pyrazole-5-carboxamide;
N—((S)-2-((5-Fluoro-4-(((S)-2-oxo-4-(trifluoromethyl)imidazolidin-1-yl)methyl)pyridin-2-yl)amino)-1-((1r,4S)-4-methylcyclohexyl)-2-oxoethyl)-1-methyl-1H-pyrazole-5-carboxamide;
Tert-butyl ((S)-2-((4-((S)-2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)carbamate;
Tert-butyl ((S)-2-((4-((R)-2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)carbamate;
1-Fluoro-N—((S)-1-((1r,4S)-4-methylcyclohexyl)-2-oxo-2-((4-(((S)-2-oxo-4-(trifluoromethyl)imidazolidin-1-yl)methyl)pyridin-2-yl)amino)ethyl)cyclopropane-1-carboxamide;
N—((S)-1-((1r,4S)-4-Methylcyclohexyl)-2-oxo-2-((4-(((S)-2-oxo-4-(trifluoromethyl)imidazolidin-1-yl)methyl)pyridin-2-yl)amino)ethyl)-1-(4,4,4-trifluoro-3-hydroxybutyl)-1H-pyrazole-5-carboxamide;
(4-Methyl-1,2,5-oxadiazol-3-yl)methyl ((S)-1-(4,4-difluorocyclohexyl)-2-oxo-2-((4-(((S)-2-oxo-4-(trifluoromethyl)imidazolidin-1-yl)methyl)pyridin-2-yl)amino)ethyl);
Tert-butyl ((1S)-2-((4-(cyclopropyl(4,4,4-trifluorobutanamido)methyl)pyridin-2-yl)amino)-1-((1r,4S)-4-methylcyclohexyl)-2-oxoethyl)carbamate;
Tert-butyl ((1S)-2-((4-(cyclopropyl(4,4,4-trifluorobutanamido)methyl)pyridin-2-yl)amino)-1-(4,4-difluorocyclohexyl)-2-oxoethyl)carbamate;
N-((1S)-2-((4-(2-Methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)-4-methyl-1,2,5-oxadiazole-3-carboxamide;
N-((1S)-2-((4-(2-Methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)-1-methyl-1H-pyrazole-5-carboxamide;
3-Isopropyl-N-((1S)-2-((4-(2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)isoxazole-4-carboxamide;
1-Isopropyl-N-((1S)-2-((4-(2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)-1H-pyrazole-5-carboxamide;
Tert-butyl ((S)-2-((4-((3,5-dimethyl-1H-pyrazol-4-yl)methyl)pyridin-2-yl)amino)-1-((1r,4S)-4-methylcyclohexyl)-2-oxoethyl)carbamate;
Tert-butyl ((S)-1-((1r,4S)-4-methylcyclohexyl)-2-oxo-2-((5-(((S)-2-oxo-4-(trifluoromethyl)imidazolidin-1-yl)methyl)pyridin-3-yl)amino)ethyl)carbamate;
Tert-butyl ((R)-1-((1r,4R)-4-methylcyclohexyl)-2-oxo-2-((5-(((S)-2-oxo-4-(trifluoromethyl)imidazolidin-1-yl)methyl)pyridin-3-yl)amino)ethyl)carbamate;
Benzyl ((rac)-3-(tert-butoxy)-1-oxo-1-((4-(((S)-2-oxo-4-(trifluoromethyl)imidazolidin-1-yl)methyl)pyridin-2-yl)amino)propan-2-yl)carbamate;
N-(3-(Tert-butoxy)-1-oxo-1-((4-(((S)-2-oxo-4-(trifluoromethyl)imidazolidin-1-yl)methyl)pyridin-2-yl)amino)propan-2-yl)-4-cyclopropyl-1,2,5-oxadiazole-3-carboxamide;
N-(3-(Tert-butoxy)-1-oxo-1-((4-(((S)-2-oxo-4-(trifluoromethyl)imidazolidin-1-yl)methyl)pyridin-2-yl)amino)propan-2-yl)-1-methyl-1H-pyrazole-5-carboxamide;
4-Cyclopropyl-N—((S)-1-((1r,4S)-4-methylcyclohexyl)-2-oxo-2-((5-(((S)-2-oxo-4-(trifluoromethyl)imidazolidin-1-yl)methyl)pyridin-3-yl)amino)ethyl)-1,2,5-oxadiazole-3-carboxamide;
4-Cyclopropyl-N—((R)-1-((1r,4R)-4-methylcyclohexyl)-2-oxo-2-((5-(((S)-2-oxo-4-(trifluoromethyl)imidazolidin-1-yl)methyl)pyridin-3-yl)amino)ethyl)-1,2,5-oxadiazole-3-carboxamide;
4-Ethyl-N-((1S)-2-((4-(2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)-1,2,5-oxadiazole-3-carboxamide;
N-((1S)-2-((4-(2-Methoxy-1-((RS)-2-oxo-4-(trifluoromethyl)imidazolidin-1-yl)ethyl)pyridin-2-yl)amino)-1-((1r,4S)-4-methylcyclohexyl)-2-oxoethyl)-2-methyl-4-(trifluoromethyl)pyrimidine-5-carboxamide;
N-((1S)-2-((4-(2-Methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)-5-methylthiazole-4-carboxamide;
5-Cyano-N-((1S)-2-((4-(2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)thiazole-4-carboxamide;
N-((1S)-2-((4-(2-Methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)-1-methyl-1H-tetrazole-5-carboxamide;
6-Bromo-N-((1S)-2-((4-(2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)-3-methylpicolinamide;
2-Chloro-N-((1S)-2-((4-(2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)-5-methylpyrimidine-4-carboxamide;
4-Cyclopropyl-N-((1S)-2-((4-(cyclopropyl(4,4,4-trifluorobutanamido)methyl)pyridin-2-yl)amino)-1-(4,4-difluorocyclohexyl)-2-oxoethyl)-1,2,5-oxadiazole-3-carboxamide;
(S)-2-(3-Benzylureido)-2-((1r,4S)-4-methylcyclohexyl)-N-(4-(((S)-2-oxo-4-(trifluoromethyl)imidazolidin-1-yl)methyl)pyridin-2-yl)acetamide;
(S)-2-(3-Benzylureido)-2-(4,4-difluorocyclohexyl)-N-(4-(((S)-2-oxo-4-(trifluoromethyl)imidazolidin-1-yl)methyl)pyridin-2-yl)acetamide;
Benzyl ((2R,3R)-3-(tert-butoxy)-1-oxo-1-((4-(((S)-2-oxo-4-(trifluoromethyl)imidazolidin-1-yl)methyl)pyridin-2-yl)amino)butan-2-yl)carbamate;
Benzyl ((2S,3R)-3-(tert-butoxy)-1-oxo-1-((4-(((S)-2-oxo-4-(trifluoromethyl)imidazolidin-1-yl)methyl)pyridin-2-yl)amino)butan-2-yl)carbamate;
N-((2S,3R)-3-(Tert-butoxy)-1-oxo-1-((4-(((S)-2-oxo-4-(trifluoromethyl)imidazolidin-1-yl)methyl)pyridin-2-yl)amino)butan-2-yl)-4-cyclopropyl-1,2,5-oxadiazole-3-carboxamide;
N-((2S,3R)-3-(Tert-butoxy)-1-oxo-1-((4-(((S)-2-oxo-4-(trifluoromethyl)imidazolidin-1-yl)methyl)pyridin-2-yl)amino)butan-2-yl)-1-methyl-1H-pyrazole-5-carboxamide;
Tert-butyl ((S)-1,1-dicyclopropyl-3-oxo-3-((4-(((S)-2-oxo-4-(trifluoromethyl)imidazolidin-1-yl)methyl)pyridin-2-yl)amino)propan-2-yl)carbamate;
N-((1S)-2-((4-(Cyclopropyl(4,4,4-trifluorobutanamido)methyl)pyridin-2-yl)amino)-1-(4,4-difluorocyclohexyl)-2-oxoethyl)oxazole-2-carboxamide;
N-((1S)-2-((4-(Cyclopropyl(4,4,4-trifluorobutanamido)methyl)pyridin-2-yl)amino)-1-((1r,4S)-4-methylcyclohexyl)-2-oxoethyl)-1-isopropyl-1H-pyrazole-5-carboxamide;
N—((S)-2-((4-((R)-Cyclopropyl(4,4,4-trifluorobutanamido)methyl)pyridin-2-yl)amino)-1-(4,4-difluorocyclohexyl)-2-oxoethyl)-1-methyl-1H-pyrazole-5-carboxamide;
N-((1S)-2-((4-(Cyclopropyl(4,4,4-trifluorobutanamido)methyl)pyridin-2-yl)amino)-1-(4,4-difluorocyclohexyl)-2-oxoethyl)-1-ethyl-1H-pyrazole-5-carboxamide;
N-((1S)-2-((4-(Cyclopropyl(4,4,4-trifluorobutanamido)methyl)pyridin-2-yl)amino)-1-(4,4-difluorocyclohexyl)-2-oxoethyl)oxazole-4-carboxamide;
N-((1S)-2-((4-(Cyclopropyl(4,4,4-trifluorobutanamido)methyl)pyridin-2-yl)amino)-1-(4,4-difluorocyclohexyl)-2-oxoethyl)-1-isopropyl-1H-pyrazole-5-carboxamide;
N-((1S)-2-((4-(Cyclopropyl(4,4,4-trifluorobutanamido)methyl)pyridin-2-yl)amino)-1-(4,4-difluorocyclohexyl)-2-oxoethyl)-1-methyl-1H-imidazole-2-carboxamide;
N-((1S)-2-((4-(2-Methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)-1-methyl-1H-imidazole-2-carboxamide;
(2S)-2-(2-(3,5-Dimethylisoxazol-4-yl)acetamido)-N-(4-(2-methoxy-1-((S)-2-oxo-4-(trifluoromethyl)imidazolidin-1-yl)ethyl)pyridin-2-yl)-2-((1r,4S)-4-methylcyclohexyl)acetamide;
(2S)—N-(4-(2-Methoxy-1-((S)-2-oxo-4-(trifluoromethyl)imidazolidin-1-yl)ethyl)pyridin-2-yl)-2-(2-(5-methyl-1H-pyrazol-3-yl)acetamido)-2-((1r,4S)-4-methylcyclohexyl)acetamide;
1-Isopropyl-N-((1S)-2-((4-(2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)-1H-imidazole-2-carboxamide;
N-((2S,3S)-3-(Tert-butoxy)-1-oxo-1-((4-(((S)-2-oxo-4-(trifluoromethyl)imidazolidin-1-yl)methyl)pyridin-2-yl)amino)butan-2-yl)-4-cyclopropyl-1,2,5-oxadiazole-3-carboxamide;
N-((1S)-2-((4-(Cyclopropyl(4,4,4-trifluorobutanamido)methyl)pyridin-2-yl)amino)-1-(4,4-difluorocyclohexyl)-2-oxoethyl)-1-isopropyl-1H-imidazole-2-carboxamide;
1-Methyl-N-((1S)-1-((1r,4S)-4-methylcyclohexyl)-2-oxo-2-((4-((2-oxopyrrolidin-3-yl)oxy)pyridin-2-yl)amino)ethyl)-1H-pyrazole-5-carboxamide;
1-Isopropyl-N-((1S)-1-((1r,4S)-4-methylcyclohexyl)-2-oxo-2-((4-((2-oxopyrrolidin-3-yl)oxy)pyridin-2-yl)amino)ethyl)-1H-pyrazole-5-carboxamide;
N—((S)-2-((4-(2-Methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)-4-methylisoxazole-3-carboxamide;
4-Cyclopropyl-N—((S)-2-((4-(2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)isoxazole-3-carboxamide;
N—((S)-2-((4-(2-Methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)-4-methyloxazole-5-carboxamide;
(S)—N-(4-(2-Methoxy-1-((S)-2-oxo-4-(trifluoromethyl)imidazolidin-1-yl)ethyl)pyridin-2-yl)-2-((1r,4S)-4-methylcyclohexyl)-2-(2-(3-methylisoxazol-5-yl)acetamido)acetamide;
1-Ethyl-N—((S)-2-((4-(2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)-1H-pyrazole-5-carboxamide;
3-Ethyl-N—((S)-2-((4-(2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)isoxazole-4-carboxamide;
3-Cyclopropyl-N—((S)-2-((4-(2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)isoxazole-4-carboxamide;
N-((2S,3R)-3-(Tert-butoxy)-1-oxo-1-((4-(((S)-2-oxo-4-(trifluoromethyl)imidazolidin-1-yl)methyl)pyridin-2-yl)amino)butan-2-yl)-1-isopropyl-1H-pyrazole-5-carboxamide;
N-((2S,3R)-3-(Tert-butoxy)-1-oxo-1-((4-(((S)-2-oxo-4-(trifluoromethyl)imidazolidin-1-yl)methyl)pyridin-2-yl)amino)butan-2-yl)-4-methyl-1,2,5-oxadiazole-3-carboxamide;
N-((2S,3R)-3-(Tert-butoxy)-1-oxo-1-((4-(((S)-2-oxo-4-(trifluoromethyl)imidazolidin-1-yl)methyl)pyridin-2-yl)amino)butan-2-yl)-4-ethyl-1,2,5-oxadiazole-3-carboxamide;
N-((2S,3R)-3-(Tert-butoxy)-1-oxo-1-((4-(((S)-2-oxo-4-(trifluoromethyl)imidazolidin-1-yl)methyl)pyridin-2-yl)amino)butan-2-yl)-3-isopropylisoxazole-4-carboxamide;
Tert-butyl ((1S)-1-(4,4-difluorocyclohexyl)-2-((4-(2-methoxy-1-(4,4,4-trifluorobutanamido)ethyl)pyridin-2-yl)amino)-2-oxoethyl)carbamate;
4-Cyclopropyl-N-((1S)-1-(4,4-difluorocyclohexyl)-2-((4-(2-methoxy-1-(4,4,4-trifluorobutanamido)ethyl)pyridin-2-yl)amino)-2-oxoethyl)-1,2,5-oxadiazole-3-carboxamide;
N-((1S)-1-(4,4-Difluorocyclohexyl)-2-((4-(2-methoxy-1-(4,4,4-trifluorobutanamido)ethyl)pyridin-2-yl)amino)-2-oxoethyl)-1-methyl-1H-pyrazole-5-carboxamide;
N-((1S)-1-(4,4-Difluorocyclohexyl)-2-((4-(2-methoxy-1-(4,4,4-trifluorobutanamido)ethyl)pyridin-2-yl)amino)-2-oxoethyl)-1-isopropyl-1H-pyrazole-5-carboxamide;
N-((1S)-1-(4,4-Difluorocyclohexyl)-2-((4-(2-methoxy-1-(4,4,4-trifluorobutanamido)ethyl)pyridin-2-yl)amino)-2-oxoethyl)-1-ethyl-1H-pyrazole-5-carboxamide;
1-Cyclopropyl-N-((1S)-1-(4,4-difluorocyclohexyl)-2-((4-(2-methoxy-1-(4,4,4-trifluorobutanamido)ethyl)pyridin-2-yl)amino)-2-oxoethyl)-1H-pyrazole-5-carboxamide;
N-((1S)-1-(4,4-Difluorocyclohexyl)-2-((4-(2-methoxy-1-(4,4,4-trifluorobutanamido)ethyl)pyridin-2-yl)amino)-2-oxoethyl)-1-methyl-1H-imidazole-2-carboxamide;
N-((1S)-1-(4,4-Difluorocyclohexyl)-2-((4-(2-methoxy-1-(4,4,4-trifluorobutanamido)ethyl)pyridin-2-yl)amino)-2-oxoethyl)-1-isopropyl-1H-imidazole-2-carboxamide;
1-(Cyclopropylmethyl)-N-((1S)-1-(4,4-difluorocyclohexyl)-2-((4-(2-methoxy-1-(4,4,4-trifluorobutanamido)ethyl)pyridin-2-yl)amino)-2-oxoethyl)-1H-pyrazole-5-carboxamide;
2,2-Difluoro-N—((S)-2-((4-((S)-2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)-2-(p-tolyl)acetamide;
2,2-Difluoro-N-((1S)-2-((4-(2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)-2-phenylacetamide;
Tert-butyl ((1S)-1-((1r,4S)-4-methylcyclohexyl)-2-oxo-2-((4-(((S)-2-oxo-4-(trifluoromethyl)-imidazolidin-1-yl)methyl-d)pyridin-2-yl)amino)ethyl)carbamate;
4-Cyclopropyl-N—((S)-1-((1r,4S)-4-methylcyclohexyl)-2-oxo-2-((4-(((3R,5S)-2-oxo-5-(trifluoromethyl)pyrrolidin-3-yl)methyl)pyridin-2-yl)amino)ethyl)-1,2,5-oxadiazole-3-carboxamide;
1-Ethyl-N—((S)-1-((1r,4S)-4-methylcyclohexyl)-2-oxo-2-((4-(((3R,5S)-2-oxo-5-(trifluoromethyl)pyrrolidin-3-yl)methyl)pyridin-2-yl)amino)ethyl)-1H-pyrazole-5-carboxamide;
1-Isopropyl-N—((S)-1-((1r,4S)-4-methylcyclohexyl)-2-oxo-2-((4-(((3R,5S)-2-oxo-5-(trifluoromethyl)pyrrolidin-3-yl)methyl)pyridin-2-yl)amino)ethyl)-1H-pyrazole-5-carboxamide;
1-Ethyl-N—((S)-1-((1r,4S)-4-methylcyclohexyl)-2-oxo-2-((4-(((3S,5S)-2-oxo-5-(trifluoromethyl)pyrrolidin-3-yl)methyl)pyridin-2-yl)amino)ethyl)-1H-pyrazole-5-carboxamide;
1-Isopropyl-N—((S)-1-((1r,4S)-4-methylcyclohexyl)-2-oxo-2-((4-(((3S,5S)-2-oxo-5-(trifluoromethyl)pyrrolidin-3-yl)methyl)pyridin-2-yl)amino)ethyl)-1H-pyrazole-5-carboxamide;
N-((1S)-1-(4,4-difluorocyclohexyl)-2-oxo-2-((4-(3,3,3-trifluoro-1-(4,4,4-trifluorobutanamido)propyl)pyridin-2-yl)amino)ethyl)-4-ethylisoxazole-5-carboxamide;
N—((S)-1-(4,4-difluorocyclohexyl)-2-oxo-2-((4-((R)-1-((S)-2-oxo-4-(trifluoromethyl)imidazolidin-1-yl)butyl)pyridin-2-yl)amino)ethyl)-4-ethylisoxazole-3-carboxamide;
N—((S)-1-(4,4-difluorocyclohexyl)-2-((4-((R)-2-methyl-1-((S)-2-oxo-4-(trifluoromethyl)imidazolidin-1-yl)propyl)pyridin-2-yl)amino)-2-oxoethyl)-4-ethylisoxazole-3-carboxamide;
N-((1S)-2-((4-(cyclopropyl(3,3,3-trifluoropropanamido)methyl)pyridin-2-yl)amino)-1-(4,4-difluorocyclohexyl)-2-oxoethyl)-1-isopropyl-1H-pyrazole-5-carboxamide;
N-((1S)-2-((4-(cyclopropyl(4,4,4-trifluorobutanamido)methyl)pyridin-2-yl)amino)-1-(4,4-difluorocyclohexyl)-2-oxoethyl)-3-ethylisoxazole-4-carboxamide;
4-Cyclopropyl-N—((S)-2-((4-(cyclopropyl((S)-2-oxo-4-(trifluoromethyl)imidazolidin-1-yl)methyl)pyridin-2-yl)amino)-1-(4,4-difluorocyclohexyl)-2-oxoethyl)-1,2,5-oxadiazole-3-carboxamide;
N-((1S)-2-((4-(cyclopropyl((S)-2-oxo-4-(trifluoromethyl)imidazolidin-1-yl)methyl)pyridin-2-yl)amino)-1-(4,4-difluoro cyclohexyl)-2-oxoethyl)-4-methyl-1,2,5-oxadiazole-3-carboxamide;
N-((1S)-2-((4-((cyclopropyl((S)-2-oxo-4-(trifluoromethyl)imidazolidin-1-yl)methyl)pyridin-2-yl)amino)-1-(4,4-difluoro cyclohexyl)-2-oxoethyl)-1-ethyl-1H-pyrazole-5-carboxamide;
N-((1S)-2-((4-(cyclopropyl((S)-2-oxo-4-(trifluoromethyl) imidazolidin-1-yl)methyl) pyridin-2-yl)amino)-1-(4,4-difluorocyclohexyl)-2-oxoethyl)-1-methyl-1H-pyrazole-5-carboxamide;
N—((S)-1-(4,4-difluorocyclohexyl)-2-oxo-2-((4-((R)-1-(4,4,4-trifluorobutanamido)ethyl)pyridin-2-yl)amino)ethyl)-1-isopropyl-1H-pyrazole-5-carboxamide;
4-Cyclopropyl-N—((S)-1-(4,4-difluorocyclohexyl)-2-((4-((R)-1-(4-methyl-2-oxo-2,3-dihydro-1H-imidazol-1-yl)ethyl)pyridin-2-yl)amino)-2-oxoethyl)-1,2,5-oxadiazole-3-carboxamide;
N-((1S)-1-(4,4-Difluorocyclohexyl)-2-oxo-2-((4-(1-((S)-2-oxo-4-(trifluoromethyl)imidazolidin-1-yl)ethyl)pyridin-2-yl)amino)ethyl)-4-methyl-1,2,5-oxadiazole-3-carboxamide;
4-cyclopropyl-N—((S)-1-(4,4-difluorocyclohexyl)-2-oxo-2-((4-(1-((S)-2-oxo-4-(trifluoromethyl)imidazolidin-1-yl)ethyl)pyridin-2-yl)amino) ethyl)-1,2,5-oxadiazole-3-carboxamide;
N-((1S)-1-(4,4-difluorocyclohexyl)-2-oxo-2-((4-(1-((S)-2-oxo-4-(trifluoromethyl) imidazolidin-1-yl)ethyl) pyridin-2-yl)amino) ethyl)-1-methyl-1H-pyrazole-5-carboxamide;
N—((S)-1-(4,4-difluorocyclohexyl)-2-oxo-2-((4-(2-((S)-2-oxo-4-(trifluoromethyl)imidazolidin-1-yl)propan-2-yl)pyridin-2-yl)amino)ethyl)-1-methyl-1H-pyrazole-5-carboxamide;
N—((S)-1-(4,4-difluorocyclohexyl)-2-oxo-2-((4-(2-((S)-2-oxo-4-(trifluoromethyl)imidazolidin-1-yl)propan-2-yl)pyridin-2-yl)amino)ethyl)-3-ethylisoxazole-4-carboxamide;
N—((S)-1-(4,4-difluorocyclohexyl)-2-oxo-2-((4-(2-((S)-2-oxo-4-(trifluoromethyl)imidazolidin-1-yl)propan-2-yl)pyridin-2-yl)amino)ethyl)-1-ethyl-1H-pyrazole-5-carboxamide;
N—((S)-1-(4,4-difluorocyclohexyl)-2-oxo-2-((4-(2-((S)-2-oxo-4-(trifluoromethyl)imidazolidin-1-yl)propan-2-yl)pyridin-2-yl)amino)ethyl)-1-isopropyl-1H-pyrazole-5-carboxamide;
N—((S)-2-((4-((S)-1-(5,5-difluoro-2-oxotetrahydropyrimidin-1(2H)-yl)-2-methoxyethyl)pyridin-2-yl)amino)-1-(4,4-difluorocyclohexyl)-2-oxoethyl)-5-ethylisoxazole-4-carboxamide;
N—((S)-2-((4-((S)-1-(5,5-difluoro-2-oxotetrahydropyrimidin-1(2H)-yl)-2-methoxyethyl)pyridin-2-yl)amino)-1-(4,4-difluorocyclohexyl)-2-oxoethyl)-4-ethylisoxazole-3-carboxamide;
N—((S)-2-((4-((S)-1-(5,5-difluoro-2-oxotetrahydropyrimidin-1(2H)-yl)-2-methoxyethyl)pyridin-2-yl)amino)-1-(4,4-difluorocyclohexyl)-2-oxoethyl)-3-ethylisoxazole-4-carboxamide;
N—((S)-2-((4-((S)-1-(5,5-difluoro-2-oxotetrahydropyrimidin-1(2H)-yl)-2-methoxyethyl)pyridin-2-yl)amino)-1-(4,4-difluorocyclohexyl)-2-oxoethyl)-1-ethyl-1H-pyrazole-5-carboxamide;
N—((S)-2-((4-((S)-1-(5,5-difluoro-2-oxotetrahydropyrimidin-1(2H)-yl)-2-methoxyethyl)pyridin-2-yl)amino)-1-(4,4-difluorocyclohexyl)-2-oxoethyl)-1-methyl-1H-pyrazole-5-carboxamide;
N—((S)-2-((4-((S)-1-(5,5-difluoro-2-oxotetrahydropyrimidin-1(2H)-yl)-2-methoxyethyl)pyridin-2-yl)amino)-1-(4,4-difluorocyclohexyl)-2-oxoethyl)-1-isopropyl-1H-pyrazole-5-carboxamide;
N—((S)-2-((4-((S)-1-(5,5-difluoro-2-oxotetrahydropyrimidin-1(2H)-yl)-2-methoxyethyl)pyridin-2-yl)amino)-1-(4,4-difluorocyclohexyl)-2-oxoethyl)-4-ethyl-1,2,5-oxadiazole-3-carboxamide;
N—((S)-2-((4-((S)-1-(5,5-difluoro-2-oxotetrahydropyrimidin-1(2H)-yl)-2-methoxyethyl)pyridin-2-yl)amino)-1-(4,4-difluorocyclohexyl)-2-oxoethyl)-4-methyl-1,2,5-oxadiazole-3-carboxamide;
4-Cyclopropyl-N—((S)-1-(4,4-difluorocyclohexyl)-2-((4-(2-methoxy-1-((3S,5S)-2-oxo-5-(trifluoromethyl)pyrrolidin-3-yl)ethyl)pyridin-2-yl)amino)-2-oxoethyl)-1,2,5-oxadiazole-3-carboxamide;
4-Cyclopropyl-N-((1S)-1-(4,4-difluorocyclohexyl)-2-((4-(2-methoxy-1-((3R,5S)-2-oxo-5-(trifluoromethyl)pyrrolidin-3-yl)ethyl)pyridin-2-yl)amino)-2-oxoethyl)-1,2,5-oxadiazole-3-carboxamide;
4-Cyclopropyl-N-((1S)-1-(4,4-difluorocyclohexyl)-2-((4-(2-methoxy-1-((3R,5S)-2-oxo-5-(trifluoromethyl)pyrrolidin-3-yl)ethyl)pyridin-2-yl)amino)-2-oxoethyl)-1,2,5-oxadiazole-3-carboxamide;
N—((S)-1-(4,4-Difluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-4-ethyl-1,2,5-oxadiazole-3-carboxamide;
N—((S)-1-(4,4-difluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-4-isopropylisoxazole-3-carboxamide;
N—((S)-1-(4,4-difluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-1-(ethyl-d5)-1H-pyrazole-5-carboxamide;
N—((S)-1-(4,4-difluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-1-(ethyl-d5)-1H-pyrazole-3-carboxamide;
4-cyclobutyl-N—((S)-1-(4,4-difluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)isoxazole-3-carboxamide;
N—((S)-1-(4,4-difluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-4-(2,2-difluoroethoxy)isoxazole-3-carboxamide;
N—((S)-1-(4,4-difluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-4-(2,2,2-trifluoroethoxy)-1,2,5-oxadiazole-3-carboxamide;
N—((S)-1-(4,4-difluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-3-methylisoxazole-4-carboxamide;
N—((S)-1-(4,4-difluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-4-isopropyl-1,2,5-oxadiazole-3-carboxamide;
N—((S)-1-(4,4-difluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-4-methylisoxazole-3-carboxamide;
N—((S)-1-(4,4-difluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-3-isopropylisoxazole-4-carboxamide;
N—((S)-1-(4,4-difluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-3-ethylisoxazole-4-carboxamide;
N—((S)-1-(4,4-difluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-4-methyl-1,2,5-oxadiazole-3-carboxamide;
4-cyclopropyl-N—((S)-1-(4,4-difluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-1,2,5-oxadiazole-3-carboxamide;
N—((S)-1-(4,4-difluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-1-ethyl-1H-pyrazole-5-carboxamide;
N—((S)-1-(4,4-difluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-1-methyl-1H-pyrazole;
N—((S)-1-(4,4-difluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-1-isopropyl-1H-pyrazole-5-carboxamide;
(S)-2-(4,4-difluorocyclohexyl)-N-(4-((S)-2-methoxy-1-((S)-2-oxo-4-(trifluoromethyl)imidazolidin-1-yl)ethyl)pyridin-2-yl)-2-(2-(6-methoxypyridin-3-yl)acetamido)acetamide;
2-(3-cyanophenyl)-N—((S)-1-(4,4-difluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)propan amide;
(S)-2-(2-(3-cyanophenyl)acetamido)-2-(4,4-difluorocyclohexyl)-N-(4-((S)-2-methoxy-1-((S)-2-oxo-4-(trifluoromethyl)imidazolidin-1-yl)ethyl)pyridin-2-yl)acetamide;
N-((1S)-1-(4,4-difluorocyclohexyl)-2-oxo-2-((4-(1-(4,4,4-trifluorobutanamido)propyl)pyridin-2-yl)amino)ethyl)-4-ethyl-1,2,5-oxadiazole-3-carboxamide;
N—((S)-1-(4,4-difluorocyclohexyl)-2-oxo-2-((4-((R)-1-((S)-2-oxo-4-(trifluoromethyl)imidazolidin-1-yl)propyl)pyridin-2-yl)amino)ethyl)-4-ethylisoxazole-3-carboxamide;
N—((S)-1-(4,4-difluorocyclohexyl)-2-oxo-2-((4-((R)-1-((S)-2-oxo-4-(trifluoromethyl)imidazolidin-1-yl)propyl)pyridin-2-yl)amino)ethyl)-4-ethyl-1,2,5-oxadiazole-3-carboxamide;
N—((S)-1-(4,4-difluorocyclohexyl)-2-oxo-2-((4-((R)-1-((S)-2-oxo-4-(trifluoromethyl)imidazolidin-1-yl)propyl)pyridin-2-yl)amino)ethyl)-3-ethylisoxazole-4-carboxamide;
N—((S)-1-(4,4-difluorocyclohexyl)-2-oxo-2-((4-((R)-1-((S)-2-oxo-4-(trifluoromethyl)imidazolidin-1-yl)propyl)pyridin-2-yl)amino)ethyl)-4-methyl-1,2,5-oxadiazole-3-carboxamide;
4-cyclopropyl-N—((S)-1-(4,4-difluorocyclohexyl)-2-oxo-2-((4-((R)-1-((S)-2-oxo-4-(trifluoromethyl)imidazolidin-1-yl)propyl)pyridin-2-yl)amino)ethyl)-1,2,5-oxadiazole-3-carboxamide;
N—((S)-1-(4,4-difluorocyclohexyl)-2-oxo-2-((4-((R)-1-((S)-2-oxo-4-(trifluoromethyl)imidazolidin-1-yl)propyl)pyridin-2-yl)amino)ethyl)-1-ethyl-1H-pyrazole-5-carboxamide;
N—((S)-1-(4,4-difluorocyclohexyl)-2-oxo-2-((4-((R)-1-((S)-2-oxo-4-(trifluoromethyl)imidazolidin-1-yl)propyl)pyridin-2-yl)amino)ethyl)-1-methyl-1H-pyrazole-5-carboxamide;
N—((S)-1-(4,4-difluorocyclohexyl)-2-oxo-2-((4-((R)-1-((S)-2-oxo-4-(trifluoromethyl)imidazolidin-1-yl)propyl)pyridin-2-yl)amino)ethyl)-1-isopropyl-1H-pyrazole-5-carboxamide;
N-((1S)-1-(4,4-difluorocyclohexyl)-2-oxo-2-((4-((2-oxo-5-(trifluoromethyl)-2,5-dihydro-1H-pyrrol-3-yl)methyl)pyridin-2-yl)amino)ethyl)-3-ethylisoxazole-4-carboxamide;
N—((S)-1-(4,4-difluorocyclohexyl)-2-oxo-2-((4-(((3S,5S)-2-oxo-5-(trifluoromethyl)pyrrolidin-3-yl)methyl)pyridin-2-yl)amino)ethyl)-4-ethyloxazole-5-carboxamide;
N—((S)-1-(4,4-difluorocyclohexyl)-2-oxo-2-((4-(((3S,5S)-2-oxo-5-(trifluoromethyl)pyrrolidin-3-yl)methyl)pyridin-2-yl)amino)ethyl)-5-ethylisoxazole-4-carboxamide;
N—((S)-1-(4,4-difluorocyclohexyl)-2-oxo-2-((4-(((3S,5S)-2-oxo-5-(trifluoromethyl)pyrrolidin-3-yl)methyl)pyridin-2-yl)amino)ethyl)-5-isopropylisoxazole-4-carboxamide;
N—((S)-1-(4,4-difluorocyclohexyl)-2-oxo-2-((4-(((3S,5S)-2-oxo-5-(trifluoromethyl)pyrrolidin-3-yl)methyl)pyridin-2-yl)amino)ethyl)-4-ethyl-1,2,5-oxadiazole-3-carboxamide;
N—((S)-1-(4,4-difluorocyclohexyl)-2-oxo-2-((4-(((3S,5S)-2-oxo-5-(trifluoromethyl)pyrrolidin-3-yl)methyl)pyridin-2-yl)amino)ethyl)-4-methyl-1,2,5-oxadiazole-3-carboxamide;
4-cyclopropyl-N—((S)-1-(4,4-difluorocyclohexyl)-2-oxo-2-((4-(((3S,5S)-2-oxo-5-(trifluoromethyl)pyrrolidin-3-yl)methyl)pyridin-2-yl)amino)ethyl)-1,2,5-oxadiazole-3-carboxamide;
N—((S)-1-(4,4-difluorocyclohexyl)-2-oxo-2-((4-(((3S,5S)-2-oxo-5-(trifluoromethyl)pyrrolidin-3-yl)methyl)pyridin-2-yl)amino)ethyl)-1-ethyl-1H-pyrazole-5-carboxamide;
N—((S)-1-(4,4-difluorocyclohexyl)-2-oxo-2-((4-(((3S,5S)-2-oxo-5-(trifluoromethyl)pyrrolidin-3-yl)methyl)pyridin-2-yl)amino)ethyl)-1-methyl-1H-pyrazole-5-carboxamide;
N—((S)-1-(4,4-difluorocyclohexyl)-2-oxo-2-((4-(((3S,5S)-2-oxo-5-(trifluoromethyl)pyrrolidin-3-yl)methyl)pyridin-2-yl)amino)ethyl)-1-isopropyl-1H-pyrazole-5-carboxamide;
N—((S)-1,1-Dicyclopropyl-3-((4-((S)-2-methoxy-1-((S)-2-oxo-4-(trifluoromethyl)imidazolidin-1-yl)ethyl)pyridin-2-yl)amino)-3-oxopropan-2-yl)-4-methyl-1,2,5-oxadiazole-3-carboxamide;
4-cyclopropyl-N—((S)-1,1-dicyclopropyl-3-((4-((S)-2-methoxy-1-((S)-2-oxo-4-(trifluoromethyl)imidazolidin-1-yl)ethyl)pyridin-2-yl)amino)-3-oxopropan-2-yl)-1,2,5-oxadiazole-3-carboxamide;
N—((S)-1,1-dicyclopropyl-3-((4-((S)-2-methoxy-1-((S)-2-oxo-4-(trifluoromethyl)imidazolidin-1-yl)ethyl)pyridin-2-yl)amino)-3-oxopropan-2-yl)-1-methyl-1H-pyrazole-5-carboxamide;
N—((S)-1,1-dicyclopropyl-3-((4-((S)-2-methoxy-1-((S)-2-oxo-4-(trifluoromethyl) imidazolidin-1-yl)ethyl) pyridin-2-yl)amino)-3-oxopropan-2-yl)-1-isopropyl-1H-pyrazole-5-carboxamide;
N—((S)-1,1-dicyclopropyl-3-oxo-3-((4-(((3S,5S)-2-oxo-5-(trifluoromethyl)pyrrolidin-3-yl)methyl)pyridin-2-yl)amino)propan-2-yl)-1-ethyl-1H-pyrazole-5-carboxamide;
4-Cyclopropyl-N-((1S)-1-((1r,4S)-4-methylcyclohexyl)-2-oxo-2-((4-(((S)-2-oxo-4-(trifluoromethyl)imidazolidin-1-yl)methyl-d)pyridin-2-yl)amino)ethyl)-1,2,5-oxadiazole-3-carboxamide;
1-Methyl-N-((1S)-1-((1r,4S)-4-methylcyclohexyl)-2-oxo-2-((4-(((S)-2-oxo-4-(trifluoromethyl)imidazolidin-1-yl)methyl-d)pyridin-2-yl)amino)ethyl)-1H-pyrazole-5-carboxamide;
4-Cyclopropyl-N-((1S)-2-((4-(2-hydroxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)-1,2,5-oxadiazole-3-carboxamide;
4-Cyclopropyl-N-((1S)-2-((4-(cyclopropyl(4,4,4-trifluorobutanamido)methyl)pyridin-2-yl)amino)-1-((1r,4S)-4-methylcyclohexyl)-2-oxoethyl)-1,2,5-oxadiazole-3-carboxamide;
N-((1S)-2-((4-(cyclopropyl(4,4,4-trifluorobutanamido)methyl)pyridin-2-yl)amino)-1-((1r,4S)-4-methylcyclohexyl)-2-oxoethyl)-1-methyl-1H-pyrazole-5-carboxamide;
4-methyl-N—((S)-1-((1r,4S)-4-methylcyclohexyl)-2-oxo-2-((4-((R)-1-((S)-2-oxo-4-(trifluoromethyl)imidazolidin-1-yl)ethyl)pyridin-2-yl)amino)ethyl)-1,2,5-oxadiazole-3-carboxamide;
4-cyclopropyl-N—((S)-1-((1r,4S)-4-methylcyclohexyl)-2-oxo-2-((4-((R)-1-((S)-2-oxo-4-(trifluoromethyl)imidazolidin-1-yl)ethyl)pyridin-2-yl)amino)ethyl)-1,2,5-oxadiazole-3-carboxamide;
1-ethyl-N—((S)-1-((1r,4S)-4-methylcyclohexyl)-2-oxo-2-((4-((R)-1-((S)-2-oxo-4-(trifluoromethyl)imidazolidin-1-yl)ethyl)pyridin-2-yl)amino)ethyl)-1H-pyrazole-5-carboxamide;
1-methyl-N—((S)-1-((1r,4S)-4-methylcyclohexyl)-2-oxo-2-((4-((R)-1-((S)-2-oxo-4-(trifluoromethyl)imidazolidin-1-yl)ethyl)pyridin-2-yl)amino)ethyl)-1H-pyrazole-5-carboxamide;
1-isopropyl-N—((S)-1-((1r,4S)-4-methylcyclohexyl)-2-oxo-2-((4-((R)-1-((S)-2-oxo-4-(trifluoromethyl)imidazolidin-1-yl)ethyl)pyridin-2-yl)amino)ethyl)-1H-pyrazole-5-carboxamide;
N-((1S)-2-((4-(2-methoxy-1-(4,4,4-trifluorobutanamido)ethyl)pyridin-2-yl)amino)-1-((1r,4S)-4-methylcyclohexyl)-2-oxoethyl)-4-methyl-1,2,5-oxadiazole-3-carboxamide;
4-cyclopropyl-N-((1S)-2-((4-(2-methoxy-1-(4,4,4-trifluorobutanamido)ethyl)pyridin-2-yl)amino)-1-((1r,4S)-4-methylcyclohexyl)-2-oxoethyl)-1,2,5-oxadiazole-3-carboxamide;
1-ethyl-N-((1S)-2-((4-(2-methoxy-1-(4,4,4-trifluorobutanamido)ethyl)pyridin-2-yl)amino)-1-((1r,4S)-4-methylcyclohexyl)-2-oxoethyl)-1H-pyrazole-5-carboxamide;
N-((1S)-2-((4-(2-methoxy-1-(4,4,4-trifluorobutanamido)ethyl)pyridin-2-yl)amino)-1-((1r,4S)-4-methylcyclohexyl)-2-oxoethyl)-1-methyl-1H-pyrazole-5-carboxamide;
1-isopropyl-N-((1S)-2-((4-(2-methoxy-1-(4,4,4-trifluorobutanamido)ethyl)pyridin-2-yl)amino)-1-((1r,4S)-4-methylcyclohexyl)-2-oxoethyl)-1H-pyrazole-5-carboxamide;
2-(3-Cyanophenyl)-2,2-difluoro-N—((S)-2-((4-((S)-2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)acetamide;
2,2-difluoro-N—((S)-2-((4-((S)-2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)-2-(1-methyl-1H-pyrazol-5-yl)acetamide;
2-(3-chlorophenyl)-2,2-difluoro-N—((S)-2-((4-((S)-2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)acetamide;
2-(3,5-difluorophenyl)-2,2-difluoro-N—((S)-2-((4-((S)-2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)acetamide;
2,2-difluoro-N—((S)-2-((4-((S)-2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)-2-(pyrazin-2-yl)acetamide;
2,2-difluoro-N—((S)-2-((4-((S)-2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)-2-(6-methoxypyridin-3-yl)acetamide;
(S)—N-(4-((S)-2-methoxy-1-((S)-2-oxo-4-(trifluoromethyl)imidazolidin-1-yl)ethyl)pyridin-2-yl)-2-((1r,4S)-4-methylcyclohexyl)-2-(2-(5-methylisoxazol-3-yl)acetamido)acetamide;
2-fluoro-N—((S)-2-((4-((S)-2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)-2-phenylacetamide;
2,2-difluoro-N—((S)-2-((4-((S)-2-methoxy-1-((S)-2-oxo-4-(trifluoromethyl)imidazolidin-1-yl)ethyl)pyridin-2-yl)amino)-1-((1r,4S)-4-methylcyclohexyl)-2-oxo ethyl)-3-methylbutanamide;
4-methyl-N—((S)-1-((1r,4S)-4-methylcyclohexyl)-2-oxo-2-((4-((R)-1-((S)-2-oxo-4-(trifluoromethyl)imidazolidin-1-yl)propyl)pyridin-2-yl)amino)ethyl)-1,2,5-oxadiazole-3-carboxamide;
4-cyclopropyl-N—((S)-1-((1r,4S)-4-methylcyclohexyl)-2-oxo-2-((4-((R)-1-((S)-2-oxo-4-(trifluoromethyl)imidazolidin-1-yl)propyl)pyridin-2-yl)amino)ethyl)-1,2,5-oxadiazole-3-carboxamide;
1-ethyl-N—((S)-1-((1r,4S)-4-methylcyclohexyl)-2-oxo-2-((4-((R)-1-((S)-2-oxo-4-(trifluoromethyl)imidazolidin-1-yl)propyl)pyridin-2-yl)amino)ethyl)-1H-pyrazole-5-carboxamide;
1-methyl-N—((S)-1-((1r,4S)-4-methylcyclohexyl)-2-oxo-2-((4-((R)-1-((S)-2-oxo-4-(trifluoromethyl)imidazolidin-1-yl)propyl)pyridin-2-yl)amino)ethyl)-1H-pyrazole-5-carboxamide;
1-isopropyl-N—((S)-1-((1r,4S)-4-methylcyclohexyl)-2-oxo-2-((4-((R)-1-((S)-2-oxo-4-(trifluoromethyl)imidazolidin-1-yl)propyl)pyridin-2-yl)amino)ethyl)-1H-pyrazole-5-carboxamide;
N—((S)-1-(3,3-difluorocyclobutyl)-2-((4-((S)-2-methoxy-1-((S)-2-oxo-4-(trifluoromethyl)imidazolidin-1-yl)ethyl)pyridin-2-yl)amino)-2-oxoethyl)-4-ethylisoxazole-3-carboxamide;
N—((S)-1-(3,3-difluorocyclobutyl)-2-((4-((S)-2-methoxy-1-((S)-2-oxo-4-(trifluoromethyl)imidazolidin-1-yl)ethyl)pyridin-2-yl)amino)-2-oxoethyl)-1-isopropyl-1H-pyrazole-5-carboxamide;
4-Cyclopropyl-N—((S)-1-((1s,4R)-4-methylcyclohexyl)-2-oxo-2-((4-(((S)-2-oxo-4-(trifluoromethyl)imidazolidin-1-yl)methyl)pyridin-2-yl)amino)ethyl)-1,2,5-oxadiazole-3-carboxamide;
1-Methyl-N—((S)-1-((1s,4R)-4-methylcyclohexyl)-2-oxo-2-((4-(((S)-2-oxo-4-(trifluoromethyl)imidazolidin-1-yl)methyl)pyridin-2-yl)amino)ethyl)-1H-pyrazole-5-carboxamide;
N—((S)-1-((4-((S)-2-methoxy-1-((S)-2-oxo-4-(trifluoromethyl)imidazolidin-1-yl)ethyl)pyridin-2-yl)amino)-1-oxo-3-((1,1,1-trifluoro-2-methylpropan-2-yl)oxy)propan-2-yl)-4-methylisoxazole-3-carboxamide;
4-ethyl-N—((S)-1-((4-((S)-2-methoxy-1-((S)-2-oxo-4-(trifluoromethyl)imidazolidin-1-yl)ethyl)pyridin-2-yl)amino)-1-oxo-3-((1,1,1-trifluoro-2-methylpropan-2-yl)oxy)propan-2-yl)-1,2,5-oxadiazole-3-carboxamide;
3-ethyl-N—((S)-1-((4-((S)-2-methoxy-1-((S)-2-oxo-4-(trifluoromethyl)imidazolidin-1-yl)ethyl)pyridin-2-yl)amino)-1-oxo-3-((1,1,1-trifluoro-2-methylpropan-2-yl)oxy)propan-2-yl)isoxazole-4-carboxamide;
N—((S)-1-((4-((S)-2-methoxy-1-((S)-2-oxo-4-(trifluoromethyl)imidazolidin-1-yl)ethyl)pyridin-2-yl)amino)-1-oxo-3-((1,1,1-trifluoro-2-methylpropan-2-yl)oxy)propan-2-yl)-4-methyl-1,2,5-oxadiazole-3-carboxamide;
4-cyclopropyl-N—((S)-1-((4-((S)-2-methoxy-1-((S)-2-oxo-4-(trifluoromethyl) imidazolidin-1-yl)ethyl)pyridin-2-yl)amino)-1-oxo-3-((1,1,1-trifluoro-2-methylpropan-2-yl)oxy)propan-2-yl)-1,2,5-oxadiazole-3-carboxamide;
1-ethyl-N—((S)-1-((4-((S)-2-methoxy-1-((S)-2-oxo-4-(trifluoromethyl)imidazolidin-1-yl)ethyl)pyridin-2-yl)amino)-1-oxo-3-((1,1,1-trifluoro-2-methylpropan-2-yl)oxy)propan-2-yl)-1H-pyrazole-5-carboxamide;
N—((S)-1-((4-((S)-2-methoxy-1-((S)-2-oxo-4-(trifluoromethyl)imidazolidin-1-yl)ethyl)pyridin-2-yl)amino)-1-oxo-3-((1,1,1-trifluoro-2-methylpropan-2-yl)oxy)propan-2-yl)-1-methyl-1H-pyrazole-5-carboxamide;
1-isopropyl-N—((S)-1-((4-((S)-2-methoxy-1-((S)-2-oxo-4-(trifluoromethyl)imidazolidin-1-yl)ethyl)pyridin-2-yl)amino)-1-oxo-3-((1,1,1-trifluoro-2-methylpropan-2-yl)oxy)propan-2-yl)-1H-pyrazole-5-carboxamide;
4-methyl-N—((S)-1-oxo-1-((4-(((S)-2-oxo-4-(trifluoromethyl)imidazolidin-1-yl)methyl)pyridin-2-yl)amino)-3-((1,1,1-trifluoro-2-methylpropan-2-yl)oxy)propan-2-yl)-1,2,5-oxadiazole-3-carboxamide;
4-cyclopropyl-N—((S)-1-oxo-1-((4-(((S)-2-oxo-4-(trifluoromethyl)imidazolidin-1-yl)methyl)pyridin-2-yl)amino)-3-((1,1,1-trifluoro-2-methylpropan-2-yl)oxy)propan-2-yl)-1,2,5-oxadiazole-3-carboxamide;
1-methyl-N—((S)-1-oxo-1-((4-(((S)-2-oxo-4-(trifluoromethyl)imidazolidin-1-yl)methyl)pyridin-2-yl)amino)-3-((1,1,1-trifluoro-2-methylpropan-2-yl)oxy)propan-2-yl)-1H-pyrazole-5-carboxamide;
4-Cyclopropyl-N-(1-(4-fluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-1,2,5-oxadiazole-3-carboxamide;
1-ethyl-N-(1-(4-fluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-1H-pyrazole-5-carboxamide;
4-(2,2-difluoroethoxy)-N-(1-(4-fluorocyclohexyl)-2-((4-((S)-2-methoxy-1-((S)-2-oxo-4-(trifluoromethyl) imidazolidin-1-yl)ethyl)pyridin-2-yl)amino)-2-oxoethyl)isoxazole-3-carboxamide;
N-(1-(4-fluorocyclohexyl)-2-((4-((S)-2-methoxy-1-((S)-2-oxo-4-(trifluoromethyl) imidazolidin-1-yl)ethyl)pyridin-2-yl)amino)-2-oxoethyl)-4-(2,2,2-trifluoroethoxy)-1,2,5-oxadiazole-3-carboxamide;
N-(1-(4-fluorocyclohexyl)-2-((4-((S)-2-methoxy-1-((S)-2-oxo-4-(trifluoromethyl) imidazolidin-1-yl)ethyl)pyridin-2-yl)amino)-2-oxoethyl)-3-methylisoxazole-4-carboxamide;
N-(1-(4-fluorocyclohexyl)-2-((4-((S)-2-methoxy-1-((S)-2-oxo-4-(trifluoromethyl) imidazolidin-1-yl)ethyl)pyridin-2-yl)amino)-2-oxoethyl)-4-methylisoxazole-3-carboxamide;
4-ethyl-N-(1-(4-fluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-1,2,5-oxadiazole-3-carboxamide;
3-ethyl-N-(1-(4-fluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)isoxazole-4-carboxamide;
N-(1-(4-fluorocyclohexyl)-2-((4-((S)-2-methoxy-1-((S)-2-oxo-4-(trifluoromethyl) imidazolidin-1-yl)ethyl)pyridin-2-yl)amino)-2-oxoethyl)-4-methyl-1,2,5-oxadiazole-3-carboxamide;
N-(1-(4-fluorocyclohexyl)-2-((4-((S)-2-methoxy-1-((S)-2-oxo-4-(trifluoromethyl) imidazolidin-1-yl)ethyl)pyridin-2-yl)amino)-2-oxoethyl)-1-methyl-1H-pyrazole-5-carboxamide;
N-(1-(4-fluorocyclohexyl)-2-((4-((S)-2-methoxy-1-((S)-2-oxo-4-(trifluoromethyl) imidazolidin-1-yl)ethyl)pyridin-2-yl)amino)-2-oxoethyl)-1-isopropyl-1H-pyrazole-5-carboxamide;
4-Ethyl-N—((S)-1-((1r,4S)-4-fluorocyclohexyl)-2-oxo-2-((4-(((S)-2-oxo-4-(trifluoromethyl)imidazolidin-1-yl)methyl)pyridin-2-yl)amino)ethyl)-1,2,5-oxadiazole-3-carboxamide;
N—((S)-1-((1r,4S)-4-fluorocyclohexyl)-2-oxo-2-((4-(((S)-2-oxo-4-(trifluoromethyl)imidazolidin-1-yl)methyl)pyridin-2-yl)amino)ethyl)-4-methyl-1,2,5-oxadiazole-3-carboxamide;
4-cyclopropyl-N—((S)-1-((1r,4S)-4-fluorocyclohexyl)-2-oxo-2-((4-(((S)-2-oxo-4-(trifluoromethyl)imidazolidin-1-yl)methyl)pyridin-2-yl)amino)ethyl)-1,2,5-oxadiazole-3-carboxamide;
1-ethyl-N—((S)-1-((1r,4S)-4-fluorocyclohexyl)-2-oxo-2-((4-(((S)-2-oxo-4-(trifluoromethyl)imidazolidin-1-yl)methyl)pyridin-2-yl)amino)ethyl)-1H-pyrazole-5-carboxamide;
N—((S)-1-((1r,4S)-4-fluorocyclohexyl)-2-oxo-2-((4-(((S)-2-oxo-4-(trifluoromethyl)imidazolidin-1-yl)methyl)pyridin-2-yl)amino)ethyl)-1-methyl-1H-pyrazole-5-carboxamide;
4-Cyclopropyl-N-(1-(4-fluoro-4-methylcyclohexyl)-2-((4-((S)-2-methoxy-1-((S)-2-oxo-4-(trifluoromethyl)imidazolidin-1-yl)ethyl)pyridin-2-yl)amino)-2-oxoethyl)-1,2,5-oxadiazole-3-carboxamide;
N-(1-(4-fluoro-4-methylcyclohexyl)-2-((4-((S)-2-methoxy-1-((S)-2-oxo-4-(trifluoromethyl)imidazolidin-1-yl)ethyl)pyridin-2-yl)amino)-2-oxoethyl)-4-methyl-1,2,5-oxadiazole-3-carboxamide;
1-ethyl-N-(1-(4-fluoro-4-methylcyclohexyl)-2-((4-((S)-2-methoxy-1-((S)-2-oxo-4-(trifluoromethyl)imidazolidin-1-yl)ethyl)pyridin-2-yl)amino)-2-oxoethyl)-1H-pyrazole-5-carboxamide;
3-ethyl-N-((1S)-2-((4-((S)-2-methoxy-1-((S)-2-oxo-4-(trifluoromethyl)imidazolidin-1-yl)ethyl)pyridin-2-yl)amino)-2-oxo-1-(4-(trifluoromethyl)cyclohexyl)ethyl)isoxazole-4-carboxamide;
1-ethyl-N-((1S)-2-((4-((S)-2-methoxy-1-((S)-2-oxo-4-(trifluoromethyl)imidazolidin-1-yl)ethyl)pyridin-2-yl)amino)-2-oxo-1-(4-(trifluoromethyl)cyclohexyl)ethyl)-1H-pyrazole-5-carboxamide;
N-((1S)-2-((4-((S)-2-methoxy-1-((S)-2-oxo-4-(trifluoromethyl)imidazolidin-1-yl)ethyl)pyridin-2-yl)amino)-2-oxo-1-(4-(trifluoromethyl)cyclohexyl)ethyl)-1-methyl-1H-pyrazole-5-carboxamide;
1-isopropyl-N-((1S)-2-((4-((S)-2-methoxy-1-((S)-2-oxo-4-(trifluoromethyl)imidazolidin-1-yl)ethyl)pyridin-2-yl)amino)-2-oxo-1-(4-(trifluoromethyl)cyclohexyl)ethyl)-1H-pyrazole-5-carboxamide;
N—((S)-1-((S)-3,3-difluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-4-ethylisoxazole-3-carboxamide;
N—((S)-1-((S)-3,3-difluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-3-ethylisoxazole-4-carboxamide;
N—((S)-1-((S)-3,3-difluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-1-ethyl-1H-pyrazole-5-carboxamide;
N—((S)-1-((S)-3,3-difluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-1-isopropyl-1H-pyrazole-5-carboxamide;
N-((1S)-1-(4-chlorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-1-ethyl-1H-pyrazole-5-carboxamide;
N-((1S)-1-(4-chlorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-1-methyl-1H-pyrazole-5-carboxamide;
N-((1S)-1-(4-chlorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-1-isopropyl-1H-pyrazole-5-carboxamide;
N-((1S)-1-(4-chlorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-3-methylisoxazole-4-carboxamide;
N-((1S)-1-(4-chlorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-4-ethylisoxazole-3-carboxamide;
N-((1S)-1-(4-chlorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-4-methyl-1,2,5-oxadiazole-3-carboxamide;
N-((1S)-1-(4-chlorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-4-ethyl-1,2,5-oxadiazole-3-carboxamide;
N-((1S)-1-(4-chlorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-3-ethylisoxazole-4-carboxamide;
N—((S)-1-((1R,3s,5S)-bicyclo[3.1.0]hexan-3-yl)-2-((4-((S)-2-methoxy-1-((S)-2-oxo-4-(trifluoromethyl)imidazolidin-1-yl)ethyl)pyridin-2-yl)amino)-2-oxoethyl)-5-ethylisoxazole-4-carboxamide;
N—((S)-1-((1R,3s,5S)-bicyclo[3.1.0]hexan-3-yl)-2-((4-((S)-2-methoxy-1-((S)-2-oxo-4-(trifluoromethyl)imidazolidin-1-yl)ethyl)pyridin-2-yl)amino)-2-oxoethyl)-5-isopropylisoxazole-4-carboxamide;
1-isopropyl-N—((S)-1-((1r,4S)-4-methylcyclohexyl)-2-oxo-2-((4-(((3S,5S)-2-oxo-5-(trifluoromethyl)pyrrolidin-3-yl)oxy)pyridin-2-yl)amino)ethyl)-1H-pyrazole-5-carboxamide;
3-ethyl-N—((S)-1-((1r,4S)-4-methylcyclohexyl)-2-oxo-2-((4-(((3S,5S)-2-oxo-5-(trifluoromethyl)pyrrolidin-3-yl)oxy)pyridin-2-yl)amino)ethyl)isoxazole-4-carboxamide;
1-ethyl-N—((S)-1-((1r,4S)-4-methylcyclohexyl)-2-oxo-2-((4-(((3S,5S)-2-oxo-5-(trifluoromethyl)pyrrolidin-3-yl)oxy)pyridin-2-yl)amino)ethyl)-1H-pyrazole-5-carboxamide;
or
1-isopropyl-N—((S)-1-((1r,4S)-4-methylcyclohexyl)-2-oxo-2-((4-(((3R,5S)-2-oxo-5-(trifluoromethyl)pyrrolidin-3-yl)oxy)pyridin-2-yl)amino)ethyl)-1H-pyrazole-5-carboxamide.

The various functional groups and substituents making up the compounds of the present disclosure are typically chosen such that the molecular weight of the compound does not exceed 1000. More usually, the molecular weight of the compound will be less than 750, for example less than 700, or less than 650, or less than 600.

A suitable pharmaceutically acceptable salt of a compound of the disclosure is, for example, an acid-addition salt of a compound of the disclosure which is sufficiently basic, for example, an acid-addition salt with, for example, an inorganic or organic acid, for example hydrochloric, hydrobromic, sulfuric, phosphoric, trifluoroacetic, formic, citric, or maleic acid. In addition a suitable pharmaceutically acceptable salt of a compound of the disclosure which is sufficiently acidic is an alkali metal salt, for example a sodium or potassium salt, an alkaline earth metal salt, for example a calcium or magnesium salt, an ammonium salt, or a salt with an organic base which affords a physiologically-acceptable cation, for example a salt with methylamine, dimethylamine, trimethylamine, piperidine, morpholine, or tris-(2-hydroxyethyl)amine.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers”, and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog (Cahn, Ingold & Prelog, Angewandte Chemie Intl. Edtn (1966) 5(4), 385-415), or by the manner in which the molecule rotates the plane of polarized light and designated as dextrorotatory or levorotatory (i.e., as (+)- or (−)-isomers respectively). A chiral compound can exist as either individual enantiomer or as a mixture thereof. A mixture containing equal proportions of the enantiomers is called a “racemic mixture”.

The compounds of this disclosure may possess one or more asymmetric centers. Apart from the stereochemistry as specified in Formula I, the description or naming of a particular compound in the specification and claims is intended to include both individual enantiomers and mixtures, racemic or otherwise, thereof, at any other asymmetric centers that may be present. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the disclosure may have geometric isomeric centres (E- and Z-isomers). It is to be understood that the present disclosure encompasses all optical, diastereoisomers, and geometric isomers and mixtures thereof.

The present disclosure also encompasses compounds of the disclosure as defined herein which comprise one or more isotopic substitutions. For example, H may be in any isotopic form, including ¹H, ²H (D), and ³H (T); C may be in any isotopic form including ¹²C, ¹³C, and ¹⁴C; and O may be in any isotopic form, including ¹⁶O and ¹⁸O; and the like.

It is also to be understood that certain compounds of the disclosure may exist in solvated as well as unsolvated forms such as, for example, hydrated forms. It is to be understood that the disclosure encompasses all such solvated forms.

It is also to be understood that certain compounds of the disclosure may exhibit polymorphism, and that the disclosure encompasses all such forms.

Compounds of the disclosure may exist in a number of different tautomeric forms and references to compounds of the disclosure include all such forms. For the avoidance of doubt, where a compound can exist in one of several tautomeric forms, and only one is specifically described or shown, all others are nevertheless embraced by compounds of the disclosure. Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

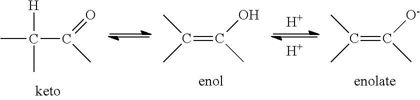

Compounds of the disclosure containing an amine function may also form N-oxides. A reference herein to a compound of the Formula I that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-Oxides can be formed by treatment of the corresponding amine with an oxidizing agent such as hydrogen peroxide or a per-acid (e.g., a peroxycarboxylic acid), see for example Advanced Organic Chemistry, by Jerry March, 4th Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (MCPBA), for example, in an inert solvent such as dichloromethane.

The compounds of the disclosure may be administered in the form of a pro-drug which is broken down in the human or animal body to release a compound of the disclosure. A pro-drug may be used to alter the physical properties or the pharmacokinetic properties of a compound of the disclosure. A pro-drug can be formed when the compound of the disclosure contains a suitable group or substituent to which a property-modifying group can be attached. Examples of pro-drugs include in vivo cleavable ester derivatives that may be formed at a carboxy group or a hydroxy group in a compound of the disclosure and in-vivo cleavable amide derivatives that may be formed at a carboxy group or an amino group in a compound of the disclosure.

Accordingly, the present disclosure includes those compounds of Formula I as defined hereinbefore when made available by organic synthesis and when made available within the human or animal body by way of cleavage of a pro-drug thereof. Accordingly, the present disclosure includes those compounds of Formula I that are produced by organic synthetic means and also such compounds that are produced in the human or animal body by way of metabolism of a precursor compound, that is a compound of the Formula I may be a synthetically-produced compound or a metabolically-produced compound.

### Synthesis

In the description of the synthetic methods described below and in the referenced synthetic methods that are used to prepare the starting materials, it is to be understood that all proposed reaction conditions, including choice of solvent, reaction atmosphere, reaction temperature, duration of the experiment, and workup procedures, can be selected by a person skilled in the art.

It is understood by one skilled in the art of organic synthesis that the functionality present on various portions of the molecule must be compatible with the reagents and reaction conditions utilised.

Necessary starting materials may be obtained by standard procedures of organic chemistry. The preparation of such starting materials is described in conjunction with the following representative process variants and within the accompanying Examples. Alternatively, necessary starting materials are obtainable by analogous procedures to those illustrated which are within the ordinary skill of an organic chemist.

It will be appreciated that during the synthesis of the compounds of the disclosure in the processes defined below, or during the synthesis of certain starting materials, it may be desirable to protect certain substituent groups to prevent their undesired reaction. The skilled chemist will appreciate when such protection is required, and how such protecting groups may be put in place and later removed.

For examples of protecting groups see one of the many general texts on the subject, for example, “Protecting groups in Organic Synthesis (3^{rd} Ed), John Wiley & Sons, NY (1999)”, T. Greene & P. Wuts. Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule.

Thus, if reactants include, for example, groups such as amino, carboxy, or hydroxy it may be desirable to protect the group in some of the reactions mentioned herein.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl, or tert-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively, an acyl group such as a tert-butoxycarbonyl group may be removed, for example, by treatment with a suitable acid as hydrochloric, sulfuric, or phosphoric acid or trifluoroacetic acid, and an arylmethoxycarbonyl group such as a benzyloxycarbonyl group may be removed, for example, by hydrogenation over a catalyst such as palladium-on-carbon, or by treatment with a Lewis acid for example BF₃·OEt₂. A suitable alternative protecting group for a primary amino group is, for example, a phthaloyl group which may be removed by treatment with an alkylamine, for example dimethylaminopropylamine, or with hydrazine.

The person skilled in the art will recognise that the compounds of the disclosure may be prepared, in known manner, in a variety of ways. Compounds of Formula I can be prepared by the methods given below, by the methods given in the experimental, or by analogous methods. The routes described are merely illustrative of some of the methods that can be employed for the synthesis of compounds of Formula I, and the person skilled in the art will appreciate that the order of the reaction steps is not limited to those described. It will also be appreciated that the assignment of nucleophile and electrophile is not limited to that described herein and in some cases it may be appropriate for the assignment to be reversed. Different approaches to synthetic chemistry strategy are described in “Organic Synthesis: The Disconnection Approach”, 2^{nd} edition, S. Warren and P. Wyatt (2008).

A compound of Formula I, or a pharmaceutically acceptable salt thereof, wherein Z, X¹, X², X³, Y, R¹, R², R³, R⁴, and R⁵ are as previously defined, may be prepared using standard acid activation methods like acid chloride, HOBt, HATU, HBTU, TOTU, EDCl, PyBOP, 1-chloro-N,N,2-trimethyl-1-propenylamine, or 1-propanephosphonic acid anhydride under basic conditions, e.g., diisopropylethylamine, triethylamine, or the like, or neutral conditions, in aprotic solvents like DMF, DMSO, DCM, acetonitrile, or the like, by coupling an activated acid (II) to the amine (III)—as shown in Scheme A. Compounds of the general formula (III) derived from compounds of the general formula (IV) can be obtained by cleaving the protecting group P, which might be an amino protecting group that is cleaved under specific conditions like BOC with HCl in dioxane or other suitable solvents, TFA without solvent or in DCM or other suitable solvents, CBZ with catalytic hydrogenation in methanol or ethanol or FMOC with secondary amines like morpholine in suitable solvents like DMF, dioxane, methanol or ethanol. Using standard amide formation methods as described above compounds of the general formula (IV) can be obtained from compounds of the general formula (VI) and amino acids of the general formula (V).

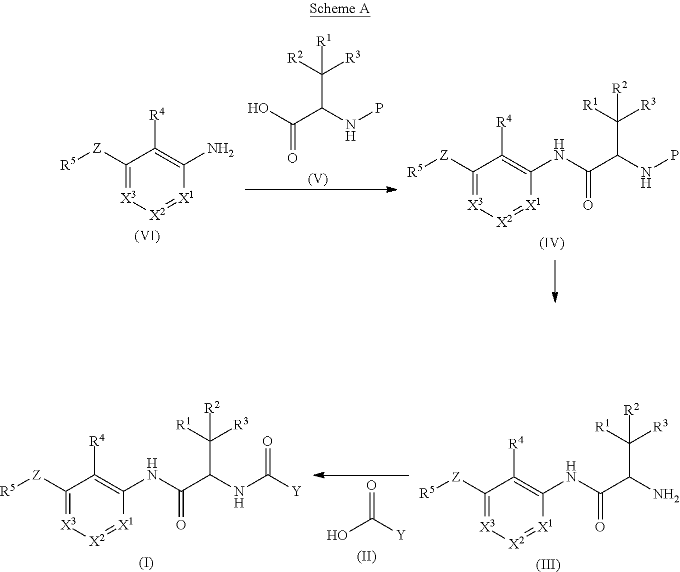

Compounds of the formula (I), wherein Z, X¹, X², X³, Y, R¹, R², R³, R⁴, and R⁵ are as previously defined, may also be prepared using standard acid activation methods like acid chloride, HOBt, HATU, HBTU, TOTU, EDCl, PyBOP, 1-chloro-N,N,2-trimethyl-1-propenylamine, or 1-propanephosphonic acid anhydride under basic conditions, e.g. diisopropylethylamine, triethylamine or the like, or neutral conditions, in aprotic solvents like DMF, DMSO, DCM, acetonitrile, or the like, by coupling an activated acid (VII) to the amine (VI)—as shown in Scheme B. Compounds of the general formula (VII) can be obtained from esters of the general formula (VIII) with alkyl groups (Alk) being preferably methyl or ethyl, that are preferably cleaved by lithium, sodium or potassium hydroxide or tert-butyl that is preferably cleaved by HCl in dioxane, TFA or TFA/DCM. Amides of the general formula (VIII) can be obtained by coupling acids of the general formula (II) with amino acid esters of the general formula (IX) using methods described above.

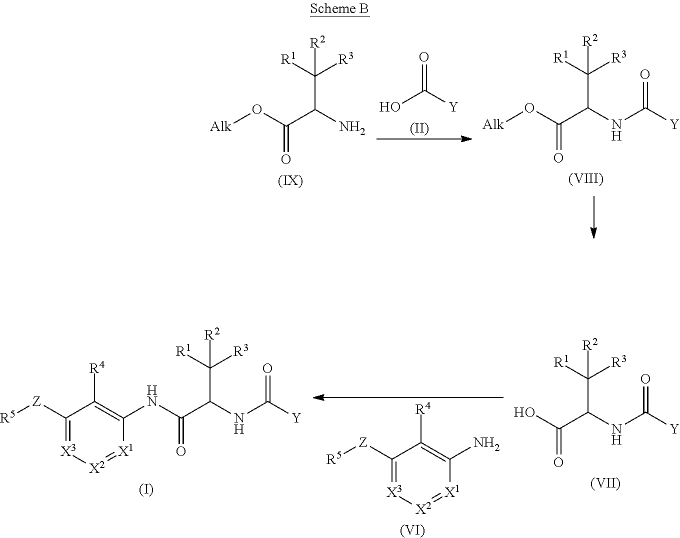

The preparation of compounds of the general formula (VI) is illustrated by the preparation of compounds of the general formula (VIa) in Scheme C. Compounds of the general formula (VIa) are obtained by cleavage of the respective protecting group P of compounds of the general formula (Xa) under conditions as described in Scheme A. Compounds of the general formula (Xa) can be obtained by cyclisation of compounds of the general formula (XIa) in the presence of carbonyl donating reagents like CDI, phosgene, triphosgene and the like, in an aprotic solvent like THF, dioxane, or DMF preferably in the presence of a base like TEA or DIPEA and at temperatures ranging between RT and 80° C., preferably 60-70° C.

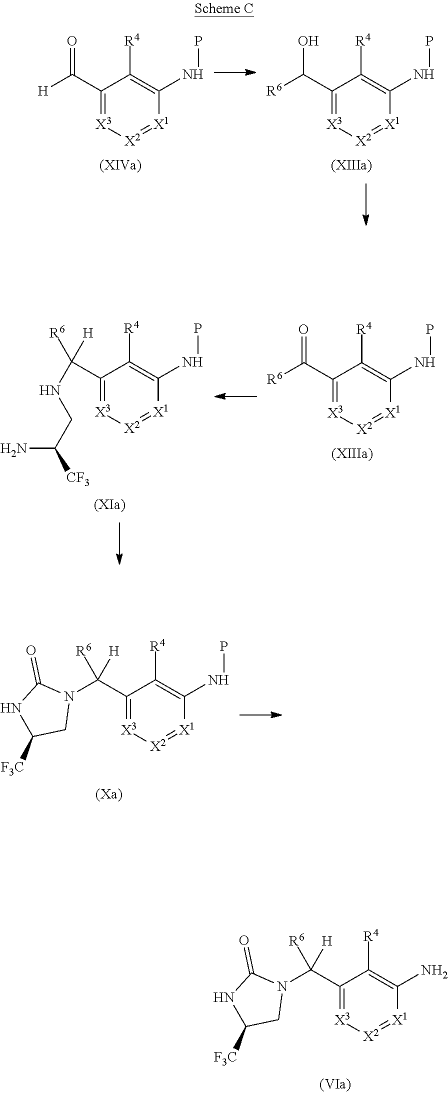

Compounds of the general formula (XIa) can be synthesised from compounds of the general formula (XIIa) and (2S)-3,3,3-trifluoropropane-1,2-diamine dihydrochloride by reductive amination in a two-step one-pot procedure. In the first step the imine is formed in protic or aprotic solvents like isopropanol or DCM at temperatures ranging from RT to 40 (DCM) or 80° C. (isopropanol) in the presence of a base like TEA or DIPEA, especially if the amine is used as a salt like its hydrochloride salt. After imine formation, the solvent is removed and the residue is dissolved in protic solvents like methanol or ethanol, and an acid like acetic acid is added to adjust the pH around 5. As reducing agents borohydrates may be used, preferably sodium cyanoborohydride or sodium triacetoxyborohydride. Compounds of the general formula (XIIa) are either commercially available or accessible from compounds of the general formula (XIIIa) by oxidation using standard oxidation means like chromic acid and TEMPO, or as described by Swern or Dess-Martin. Compounds of the general formula (XIIIa) can be obtained by reacting compounds of the general formula (XIVa), which are commercially available or accessible by known methods, with organometallic compounds (e.g. R⁶MX) such as Grignard reagents or lithium organics like nBuLi or the like, under inert conditions, and a broad temperature range from −78° C. to 100° C. in inert solvents like THE or toluene.

### Pharmaceutical Compositions

The compounds of the disclosure will normally, but not necessarily, be formulated into pharmaceutical compositions prior to administration to a patient. Therefore, according to a further aspect of the disclosure there is provided a pharmaceutical composition which comprises a compound of the disclosure as defined herein, or a pharmaceutically acceptable salt thereof, and one or more pharmaceutically acceptable excipients, diluents, or carriers.

The pharmaceutical compositions of the disclosure may be prepared and packaged in bulk form wherein a safe and effective amount of a compound of the disclosure can be extracted and then given to the patient such as with powders or syrups. Alternatively, the pharmaceutical compositions of the disclosure may be prepared and packaged in unit dosage form wherein each physically discrete unit contains a safe and effective amount of a compound of the disclosure. When prepared in unit dosage form, the pharmaceutical compositions of the disclosure typically contain from 1 mg to 1000 mg.

The compositions of the disclosure may be in a form suitable for oral use (for example as tablets, capsules, caplets, pills, troches, powders, syrups, elixirs, suspensions, solutions, emulsions, sachets, and cachets), for topical use (for example as creams, ointments, lotions, solutions, pastes, sprays, foams, and gels), for transdermal administration (for example via transdermal patches), for administration by inhalation (for example as a dry powders, aerosols, suspensions, and solutions), for administration by insufflation (for example as a finely divided powder), or for parenteral administration (for example as a sterile aqueous or oily solution for intravenous, subcutaneous, intramuscular, intraperitoneal or intramuscular dosing, or as a suppository for rectal dosing).

As used herein, “pharmaceutically acceptable excipient” means a pharmaceutically acceptable material, composition, or vehicle involved in giving form or consistency to the pharmaceutical composition. Each excipient must be compatible with the other ingredients of the pharmaceutical composition when commingled such that interactions which would substantially reduce the efficacy of the compound of the disclosure when administered to a patient, and interactions which would result in pharmaceutical compositions that are not pharmaceutically acceptable, are avoided. In addition, each excipient must of course be of sufficiently high purity to render it pharmaceutically acceptable.

The pharmaceutical compositions of the disclosure are prepared using techniques and methods known to those skilled in the art. Some of the methods commonly used in the art are described in Remington's Pharmaceutical Sciences (Mack Publishing Company).

An effective amount of a compound of the present disclosure for use in therapy of proliferative disease is an amount sufficient to symptomatically relieve in a warm-blooded animal, particularly a human, the symptoms of the proliferative disease, to slow the progression of the proliferative disease, or to reduce in patients with symptoms of the proliferative disease the risk of getting worse.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 0.5 g of active agent (more particularly from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes of a compound of Formula I will naturally vary according to the nature and severity of the conditions, the age and sex of the animal or patient, and the route of administration, according to well-known principles of medicine.

In using a compound of the disclosure for therapeutic or prophylactic purposes, it will generally be administered so that a daily dose in the range, for example, from 0.1 mg/kg to 75 mg/kg body weight is received, given if required in divided doses. In general, lower doses will be administered when a parenteral route is employed. Thus, for example, for intravenous or intraperitoneal administration, a dose in the range, for example, from 0.1 mg/kg to 30 mg/kg body weight will generally be used. Similarly, for administration by inhalation, a dose in the range, for example, from 0.05 mg/kg to 25 mg/kg body weight will be used. Oral administration may also be suitable, particularly in tablet form. Typically, unit dosage forms will contain about from 0.5 mg to 0.5 g of a compound of this disclosure.

### Routes of Administration

The compounds of the disclosure or pharmaceutical composition comprising the active compound may be administered to a subject by any convenient route of administration, whether systemically/peripherally or topically (i.e., at the site of desired action).

Routes of administration include, but are not limited to, oral (e.g., by ingestion); buccal; sublingual; transdermal (including, e.g., by a patch, plaster, etc.); transmucosal (including, e.g., by a gum, film etc.); intranasal (e.g., by nasal spray); ocular (e.g., by eyedrops); pulmonary (e.g., by inhalation or insufflation therapy using, e.g., via an aerosol, e.g., through the mouth or nose); rectal (e.g., by suppository or enema); vaginal (e.g., by pessary); parenteral (e.g., by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal); by implant of a depot or reservoir (e.g., subcutaneously or intramuscularly).

### Therapeutic Uses and Applications

The compounds of the present disclosure have been demonstrated to inhibit the binding of human IL-17A to its receptor, IL-17RA (in an AlphaLISA competition assay as described herein), with binding affinities typically being less than 30 μM. Therefore, the compounds of Formula I, being potent modulators of human IL-17A activity, are potentially beneficial as therapeutic compounds in the treatment or prevention of human ailments occurring as a result of IL-17A activity.

The compounds of the present disclosure, being high affinity binders to human IL-17A and potent modulators of human IL-17A activity, may be beneficial as pharmacological standards for use in the development of new biological tests and in the search for new pharmacological agents. Thus, the compounds of the present disclosure may be useful as radioligands in assays for detecting pharmacologically active compounds.

Thus, in one aspect, the present disclosure relates to a compound of the disclosure as defined herein, or a pharmaceutically acceptable salt or solvate thereof, or a pharmaceutical composition as defined herein, for use in therapy.

In another aspect, the present disclosure relates to a compound of the disclosure as defined herein, or a pharmaceutically acceptable salt or solvate thereof, or a pharmaceutical composition as defined herein, for use in the treatment of diseases or disorders mediated by IL-17A activity.

In another aspect, the present disclosure relates to the use of a compound of the disclosure as defined herein, or a pharmaceutically acceptable salt or solvate thereof, in the manufacture of a medicament for use in the treatment of diseases or disorders mediated by IL-17A activity.

In another aspect, the present disclosure relates to a method of treating a disease or disorder in which IL-17A activity is implicated, said method comprising administering to a subject in need of such treatment a therapeutically effective amount of a compound of the disclosure as defined herein, or a pharmaceutically acceptable salt or solvate thereof, or a pharmaceutical composition as defined herein.

Examples of particular diseases or disorders that the compounds of Formula I and their pharmaceutically acceptable salts may be used to treat include, but are not limited to, any one of the following: acute lung injury, Alzheimer's Disease, ankylosing spondylitis, axial spondyloarthritis and other spondyloarthropathies, arthritis, asthma (including severe asthma), atopic dermatitis, autoimmune diabetes, other autoimmune disorders, autoimmune thyroiditis, bone resorption, cancer (both solid tumours (such as melanomas, sarcomas, squamous cell carcinomas, transitional call cancers, and ovarian cancers) and hematologic malignancies; in particular acute myelogenous leukaemia, chronic lymphocytic leukemia, gastric cancer, and colon cancer), Castleman's disease, contact dermatitis, Crohn's Disease, chronic myelogenous leukemia, chronic obstructive pulmonary disease (COPD), coeliac disease, cystic fibrosis, dermatomyositis, discoid lupus erythematosus, eczema, enthesitis-related arthritis, endotoxic shock associated with infection, exophthalmos, fibrosing disorders including pulmonary fibrosis, gall bladder disease, giant cell arteritis, graft-versus-host disease, hepatoblastomas, hypochlorhydria, immune mediated inflammatory disorders of the central and peripheral nervous system such as multiple sclerosis and Guillain-Barr syndrome, Hidradenitis Suppurativa, inflammatory bowel disease, insulin dependent diabetes type 1, intravascular coagulation, irritable bowel syndrome, Lichen Planus, liver fibrosis, lupus nephritis, lyme arthritis, meningoencephalitis, myocarditis, meningoencephalitis, Necrobiosis Lipoidica Diabeticorum, osteoporosis, pancreatitis, Papulopustular Rosacea, Parkinson's disease, pelvic inflammatory disease, periodontitis, peritonitis, Peyronie's Disease, Pilonidal disease, psoriasis, psoriatic arthritis (PsA), Pyoderma Gangrenosum, renal fibrosis, rheumatoid arthritis, scleroderma or systemic sclerosis, stroke, surgical adhesions, systemic lupus erythematosus (SLE), systemic onset juvenile idiopathic arthritis (JIA), trauma (surgery), transplant rejection, Type I diabetes, ulcerative colitis, uveitis, and vasculitis.

Modulators of IL-17 activity may be administered to inhibit or reduce the severity of ocular inflammatory disorders (WO 2009/089036), for example ocular surface inflammatory disorders including Dry Eye Syndrome (DES). Consequently, the compounds in accordance with the present disclosure are useful in the treatment or prevention of an IL-17-mediated ocular inflammatory disorder, for example an IL-17-mediated ocular surface inflammatory disorder including Dry Eye Syndrome. Ocular surface inflammatory disorders include Dry Eye Syndrome, penetrating keratoplasty, corneal transplantation, lamellar or partial thickness transplantation, selective endothelial transplantation, corneal neovascularization, keratoprosthesis surgery, corneal ocular surface inflammatory disorders, conjunctival scarring disorders, ocular autoimmune disorders, Pemphigoid syndrome, Stevens-Johnson syndrome, ocular allergy, severe allergic (atopic) eye disease, conjunctivitis, and microbial keratitis. Particular categories of Dry Eye Syndrome include keratoconjunctivitis sicca (KCS), Sjogren syndrome, Sjogren syndrome-associated keratoconjunctivitis sicca, non-Sjogren syndrome-associated keratoconjunctivitis sicca, keratitis sicca, sicca syndrome, xerophthalmia, tear film disorder, decreased tear production, aqueous tear deficiency (ATD), meibomian gland dysfunction, and evaporative loss.

### Combination Therapies

The compounds of the disclosure may be administered alone as a monotherapy, or they may be administered in combination with one or more additional therapeutic agents. The selection of the one or more additional therapeutic agents will of course vary depending on the disease or condition to be treated and its severity.

It is commonplace to use combination therapies to treat certain medical conditions.

According to a particular aspect of the disclosure there is provided a combination suitable for use in the treatment of a disease or condition in which IL-17 activity is implicated, comprising a compound of the disclosure as defined hereinbefore, or a pharmaceutically acceptable salt thereof, and another therapeutic agent.

In a further aspect of the disclosure there is provided a compound of the disclosure or a pharmaceutically acceptable salt thereof, in combination with one or more additional therapeutic agents.

According to a further aspect of the disclosure there is provided a pharmaceutical composition which comprises a compound of the disclosure, or a pharmaceutically acceptable salt thereof in combination with one or more additional therapeutic agents in association with a pharmaceutically acceptable diluent or carrier.

The one or more additional therapeutic agents may comprise a further compound of the present disclosure. Therefore, in an embodiment, there is provided a pharmaceutical composition which comprises two compounds of the disclosure, or pharmaceutically acceptable salts thereof, in association with a pharmaceutically acceptable diluent or carrier.

The combinations referred to above may conveniently be presented for use in the form of a pharmaceutical formulation and thus pharmaceutical formulations comprising a combination as defined above together with a pharmaceutically acceptable diluent or carrier represent a further aspect of the disclosure.

Such combination treatment may be achieved by way of the simultaneous, sequential, or separate dosing of the individual components of the treatment. In one embodiment, the individual compounds will be administered simultaneously in a combined pharmaceutical formulation.

Such combination therapies employ the compounds of this disclosure within the dosage range described herein and the other pharmaceutically active agent within approved dosage ranges or the dosage such as described in the relevant publication reference.

### EXAMPLES

### General Procedures

Methods for preparing the compounds of this disclosure are illustrated in the following Examples. Starting materials are made according to procedures known in the art or as illustrated herein or are available commercially. Commercial reagents were used without further purification. Where no reaction temperature is included, the reaction was performed at ambient temperature which is typically 17-27° C.

A person skilled in the art will appreciate that reaction temperatures, reaction times and reagent quantities may be varied from those stated herein.

Where compounds described in the disclosure are characterized by ¹H NMR spectroscopy, spectra were recorded on Bruker AVANCE II 400 (400 MHz), Bruker AVANCE III HD (400 MHz), Bruker AVANCE NEO (400 MHz), or Bruker AVANCE III (600 MHz) instruments. The instruments were equipped with 5 mm BBI room temperature probe heads. Where no temperature is included, the spectra were recorded at ambient temperature. Chemical shift values are expressed in parts per million (ppm). The following abbreviations are used for the multiplicity of the NMR signals: s=singlet, d=doublet, t=triplet, q=quartet, quin=quintet, m=multiplet, br=broad.

Where compounds described in the disclosure are characterized by LCMS data, retention time and molecular weight are determined using the conditions listed below.

Method A: For retention time and mass detection, a LC/MS-system from Waters (UPLC/SQD; ionization: electrospray in positive and/or negative mode [ES+/−]) was used. Detected masses are given in mass per charge [m/z]. Waters ACQUITY UPLC BEH C18, 1.7 μm; 2.1 mm×50 mm; H₂O+0.05% FA:ACN+0.035% FA; 98:2 (0 min) to 98:2 (0.2 min) to 2:98 (3.8 min) to 2:98 (4.3 min) to 98:2 (4.5 min), 1 ml/min 55° C.; given are the RT in min at 220 nm and the observed m/z mass for the respective UV peak.

Method B: For retention time and mass detection, a LC/MS-system from Agilent (LC 1200 Series/MS 6120 quadrupole LC/MS, LC 1260 infinity/MS 6120 quadrupole LC/MS or LC 1260 Infinity II/MSD Infinity Lab) was used. Ionization: electrospray in positive mode (ES+). Detected masses are given in mass per charge [m/z]. Luna C18, 3 μm; 2.0 mm×10 mm; H₂O+0.05% TFA:ACN; 93:7 (0 min) to 5:95 (1.0 min) to 5:95 (1.45 min), 1.1 ml/min; 30° C.; given are the RT in min at 220 nm and the observed m/z mass for the respective UV peak.

Method C: For retention time and mass detection, a LC/MS-system from Shimadzu (LCMS-2020; Software: LabSolution Version 5.97SP1) was used. Ionization: electrospray in positive mode (ES+). Detected masses are given in mass per charge [m/z]. Kinetex® EVO C18, 5 μm; 2.1 mm×30 mm; 5% ACN (0.01875% TFA) in water (0.0375% TFA) to 95% ACN in water in 0.60 min, then hold at 95% ACN for 0.18 min; 2.0 ml/min; T 50° C.; given are the RT in min at 220 nm and the observed m/z mass for the respective UV peak.

Method D: For retention time and mass detection, a LC/MS-system from Shimadzu (LCMS-2020; Software: LabSolution Version 5.97SP1) was used. Ionization: electrospray in positive mode (ES+). Detected masses are given in mass per charge [m/z]. Kinetex® EVO C18, 5 μm; 2.1 mm×30 mm; 5% ACN in water (NH₄HCO₃-10 mmol/I) to 95% ACN in water in 0.8 min at 1.5 ml/min; then hold at 95% ACN for 0.15 min at 2.0 ml/min; T 40° C.; given are the RT in min at 220 nm and the observed m/z mass for the respective UV peak.

Method E: For retention time and mass detection a LC/MS-system from Shimadzu (LCMS-2020; Software: LabSolution Version 5.97SP1) was used. Ionization: electrospray in positive mode (ES+). Detected masses are given in mass per charge [m/z]. Kinetex® EVO C18, 5 μm; 2.1 mm×30 mm; 5% ACN (0.01875% TFA) in water (0.0375% TFA) to 95% ACN in water in 0.80 min (flow 1.5 ml/min), then hold at 95% ACN for 0.15 min (2.0 ml/min), in 0.01 min to 5% ACN, then hold for 0.04 min; T 50° C.; given are the RT in min at 220 nm and the observed m/z mass for the respective UV peak.

Silica gel chromatography was performed using CombiFlash® Rf (Teledyne ISCO), Büchi Reveleris® X2 or Biotage Dalton 2000 equipment with pre-packed cartridges.

Preparative reversed phase liquid chromatography was performed with a Biotage equipment using 018 columns and a water (0.1% FA)/ACN gradient.

For preparative reversed phase HPLC, an Agilent 1200 preparative HPLC machine, Gilson equipment (GX-271 liquid handler, 331/332-pump, UV/VIS-155) or a Waters Autopurification Li Prep System was used.

For preparative SFC, Waters SF0150mgm and Waters SF0350 equipment was used.

For analytical SF0, SHIMADZU LC-30AD sf equipment was used.

In compounds described as HCl—, TFA- or as another salt, the exact amount of the respective salt is usually not determined unless otherwise noted. Therefore, the amount of the salt can range from as low as 0.01 eq. up to 5.0 eq. depending on the chemical structure (e.g., number of basic centres).

Compound names were generated using Perkin Elmer's ChemDraw®, version 20.1.0.110.

### Abbreviations

| | |
|---|---|
| ACN | acetonitrile |
| BOC | tert-butyloxycarbonyl |
| BOC₂O | di-tert-butyl dicarbonate |
| BPD | Bis(pinacolato)diboron |
| CBZ | carboxybenzyl |
| CDI | carbonyldiimidazole |
| conc. | concentrated |
| dba | dibenzylideneacetone |
| DCM | dichloromethane |
| DEA | diethylamine |
| DIPEA | N,N-diisopropylethylamine |
| DMAP | 4-dimethylaminopyridine |
| DMF | N,N-dimethylformamide |
| DMP | Dess-Martin periodinane |
| DMSO | Dimethyl sulfoxide |
| DMSO-d₆ | deuterated dimethyl sulfoxide |
| DS | diastereomer |
| EA | ethyl acetate |
| EDCI | 1-ethyl-3-(3-dimethylaminopropyl)carbodiimide |
| EtOH | ethanol |
| FC | flash chromatography |
| FMOC | fluorenylmethoxycarbonyl |
| h | hour |
| HATU | O-(7-Azabenzotriazol-1-yl)-N,N,N′,N′-tetramethyluronium- |
| | hexafluorophosphate |
| HBTU | 2-(1H-Benzotriazole-1-yl)-1,1,3,3-tetramethyluronium hexafluorophosphate |
| HPLC | high-performance liquid chromatography |
| HOBt | 1-Hydroxy-1H-benzotriazol hydrate |
| LC/MS | liquid chromatography/mass spectrometry |
| LDA | Lithium diisopropylamide |
| LiHMDS | lithium bis(trimethylsilyl)amide |
| IPA | 2-propanol |
| m/z | mass-to-charge ratio |
| MeOH | methanol |
| min | minute |
| MOMCl | chloromethyl methyl ether |
| MTB | methyl tert-butyl ether |
| nBuLi | n-Butyllithium |
| nHep | n-heptane |
| Pd/C | palladium on carbon |
| Pd(dppf)Cl2 | [1,1′-bis(diphenylphosphino)ferrocene]dichloropalladium(II) |
| Pd(dtbpf)Cl₂ | [1,1′-Bis (di-tert-butylphosphino)ferrocene]dichloropalladium (II) |
| PE | petroleum ether |
| PyBOP | benzotriazole-1-yl-oxy-tris-pyrrolidino-phosphonium hexafluorophosphate |
| RP | reverse phase |
| RT | room temperature |
| rt | retention time |
| RuPhos Pd G3 | 2-Dicyclohexylphosphino-2′,6′-diisopropoxy-1,1′-biphenyl)[2-(2′-amino-1,1′- |
| | biphenyl)]palladium(II)methansulfonate |
| SFC | supercritical fluid chromatography |
| T | temperature |
| T3P | 1-propanephosphonic acid anhydride (50% solution in ethyl acetate) |
| TBAF | tetrabutylammonium fluoride |
| TBDMSCl | tert-butyldimethylsilyl chloride |
| TBTU | [benzotriazol-1-yloxy(dimethylamino)methylidene]-dimethylazanium |
| | tetrafluoroborate |
| TEA | triethylamine |
| TEMPO | 2,2,6,6-tetramethylpiperidinyloxyl |
| TFA | trifluoroacetic acid |
| THF | tetrahydrofuran |
| TOTU | O-[(Ethoxycarbonyl)cyanomethylenamino]-N,N,N′,N′-tetramethyluronium |
| | tetrafluoroborate |
| TsOH | p-toluenesulfonic acid hydrate |
| XPhos | 2-dicyclohexylphosphin-2′,4′,6′-triisopropylbiphenyl |

### Intermediate 1: Tert-butyl ((S)-2-((4-((((S)-2-amino-3,3,3-trifluoropropyl)amino)-methyl)pyridin-2-yl)amino)-1-((1r,4S)-4-methylcyclohexyl)-2-oxoethyl)carbamate

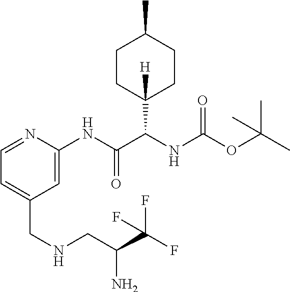

### Step A1: 4-(((Tert-butyldimethylsilyl)oxy)methyl)pyridin-2-amine

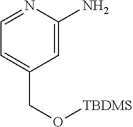

To a mixture of (2-amino-4-pyridyl)methanol (20 g) and imidazole (10.97 g) in DMF (150 ml) was added TBDMSCl (24.28 g) in one portion at 0° C. under N₂. After stirring the mixture at 20° C. for 12 h, water was added (150 ml). The mixture was extracted with EA (200 ml×3), the combined organic phases were washed with brine (100 ml×3), dried over anhydrous Na₂SO₄, filtered and concentrated in vacuo. The residue was purified by column chromatography (Silica gel, PE/EA=0/1 to 1/1) to yield 27 g of the title compound. LC/MS: m/z=239.1 [M+H]⁺; rt: 0.36 min (LC/MS-method C).

### Step A2: Tert-butyl ((S)-2-((4-(((tert-butyldimethylsilyl)oxy)methyl)pyridin-2-yl)amino)-1-((1r,4S)-4-methylcyclohexyl)-2-oxoethyl)carbamate

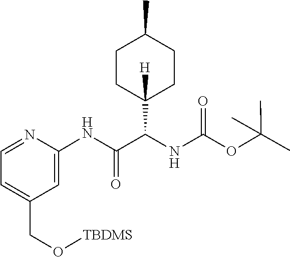

To a mixture of (2S)-2-(tert-butoxycarbonylamino)-2-(4-methylcyclohexyl)acetic acid (4.55 g) in DCM (40 ml) was added DMAP (4.10 g) and EDCl (6.43 g) at 20° C. After 5 min 4-(((tert-butyldimethylsilyl)oxy)methyl)pyridin-2-amine was added (4 g) at 20° C. After 12 h water and DCM (40 ml×3) were added. The organic phases were separated and combined, dried over Na₂SO₄, filtered and concentrated in vacuo. The residue was purified by column chromatography (Silica gel, PE/EA=0/1 to 1/1) to yield 8.2 g of the title compound. ¹H NMR (400 MHz, 00013, representative signals) 5=8.84-8.69 (m, 1H), 8.26 (dd, J=3.0, 5.0 Hz, 1H), 8.15 (s, 1H), 7.13 (d, J=5.1 Hz, 1H), 5.18 (br d, J=7.3 Hz, 1H), 4.76 (s, 2H), 4.13 (m, 1H), 1.84 (br m, 1H), 1.45 (s, 9H), 0.96 (s, 9H), 0.12 (s, 6H).

### Step A3: Tert-butyl ((S)-2-((4-(hydroxymethyl)pyridin-2-yl)amino)-1-((1r,4S)-4-methylcyclohexyl)-2-oxoethyl)carbamate

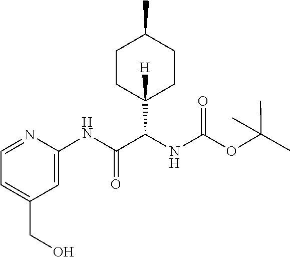

To a mixture of tert-butyl ((S)-2-((4-(((tert-butyldimethylsilyl)oxy)methyl)pyridin-2-yl)amino)-1-((1r,4S)-4-methylcyclohexyl)-2-oxoethyl)carbamate (8.20 g) in THF (82 ml) was added TBAF (1 M, 17.51 ml) in one portion at 000 under N₂. The mixture was stirred at 20° C. for 12 h. Then the reaction mixture was concentrated and the residue was purified by column chromatography (Silica gel, PE/EA=0/1 to I/O) to yield 6.29 g of the racemic title compound which was further separated by SFC (condition: Daicel chiralpak AD (30 mm*250 mm, 10 μm); mobile phases A/B: 002/0.1% NH₃—H₂O, MeOH; B %: 30%-30%, 8.5 min; 100 ml/min) to yield 2.6 g of the title compound. LC/MS: m/z=378.3 [M+H]⁺; rt: 0.39 min (LC/MS-method C). ¹H NMR (400 MHz, 00013, representative signals): δ ppm=8.79 (br s, 1H), 8.16 (d, J=5.1 Hz, 1H), 8.12 (s, 1H), 7.00 (d, J=5.0 Hz, 1H), 5.34 (br d, J=8.3 Hz, 1H), 4.64 (s, 2H), 1.33 (s, 9H). Analytical chiral analysis (column: Chiralcel OJ-3, 4.6×50 mm, 3 μm; eluents: CO₂/MeOH (0.05% DEA); gradient: B in A from 5 to 40%; flow: 3 ml/min; T: 35° C.; back pressure: 100 bar): rt: 0.57 min [For comparison: (R) enantiomer: rt: 0.70 min].

### Step A4: Tert-butyl ((S)-2-((4-formylpyridin-2-yl)amino)-1-((1r,4S)-4-methylcyclohexyl)-2-oxoethyl)carbamate

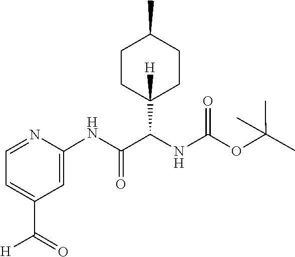

To a mixture of tert-butyl ((S)-2-((4-(hydroxymethyl)pyridin-2-yl)amino)-1-((1r,4S)-4-methylcyclohexyl)-2-oxoethyl)carbamate (2.6 g) in DCM (26 ml) DMP (4.38 g) was added in one portion at 0° C. under N₂. The mixture was stirred at 20° C. for 12 h. Then the reaction mixture was concentrated in vacuo and DCM (50 ml) and water were added.

The organic phase was separated and the aqueous phase was extracted one more time with DCM (50 ml). The combined organic phases were dried over Na₂SO₄, filtered and concentrated under reduced pressure to give a residue, which was purified by column chromatography (Silica gel, Petroleum ether/Ethyl acetate=0/1 to 1/1) to yield 2.4 g of the title compound. LC/MS: m/z=376.3/394.3 [M+H]+/[M+18+H]⁺; rt: 0.64 min (LC/MS-method D).

### Step B1: (R,E)-2,2,2-trifluoro-N-(1-phenylethyl)ethan-1-imine

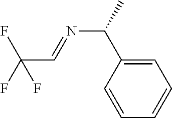

To a solution of (1R)-1-phenylethanamine (68 g) in toluene (600 ml) was added TsOH (580 mg) and 2,2,2-trifluoroethane-1,1-diol (78.14 g). The mixture was stirred at 120° C. for 12 h. The reaction mixture was diluted with water (500 ml), extracted with EA (300 ml×2), washed with brine (500 ml), dried over anhydrous Na₂SO₄, filtered and concentrated under reduced pressure to give the title compound (110 g) as a yellow oil, which was used for the next step without further purification. ¹H NMR (400 MHz, CDCl₃): δ ppm=7.70-7.61 (m, 1H), 7.43-7.28 (m, 5H), 4.62 (q, J=6.6 Hz, 1H), 1.60 (d, J=6.7 Hz, 3H).

### Step B2: (S)-1,1,1-trifluoro-3-nitro-N—((R)-1-phenylethyl)propan-2-amine

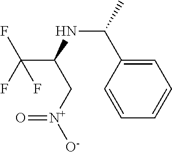 [SMILES: C[C@@H](N[C@@H](C[N+](=O)[O-])C(F)(F)F)c1ccccc1]

A mixture of (R,E)-2,2,2-trifluoro-N-(1-phenylethyl)ethan-1-imine (110 g), nitromethane (166.87 g), and ZrCl₄ (63.71 g) was stirred at 25° C. for 3 h. The reaction mixture was diluted with water (500 ml), extracted with EA (300 ml×3), dried over anhydrous Na₂SO₄, filtered and concentrated under reduced pressure to give a residue, which was purified several times by flash silica gel chromatography (PE:EA=1:0-20:1) to yield 56.5 g of the title compound. ¹H NMR (400 MHz, CDCl₃): β ppm=7.40-7.28 (m, 5H), 4.66 (dd, J=4.5, 12.8 Hz, 1H), 4.45 (dd, J=7.9, 12.7 Hz, 1H), 4.09-4.01 (m, 1H), 4.01-3.91 (m, 1H), 1.78 (br d, J=9.7 Hz, 1H), 1.36 (d, J=6.4 Hz, 3H).

### Step B3: Di-tert-butyl (3,3,3-trifluoropropane-1,2-diyl)(S)-dicarbamate

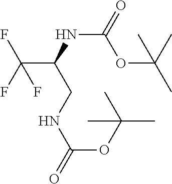 [SMILES: CC(C)(C)OC(=O)NC[C@H](NC(=O)OC(C)(C)C)C(F)(F)F]

To a solution of (S)-1,1,1-trifluoro-3-nitro-N—((R)-1-phenylethyl)propan-2-amine (46 g) in MeOH (500 ml) was added Pd/C (9 g, 10% purity) and Boc₂O (84.23 g) under N₂ atmosphere. The suspension was degassed and purged with H₂ for 3 times. The mixture was stirred under H₂ (50 psi) at 50° C. for 48 h. The reaction mixture was filtered and concentrated under reduced pressure to give the crude, then triturated with PE (100 ml) at 25° C. for 30 min to give 40.8 g of the title compound. ¹H NMR (400 MHz, CDCl₃): β ppm=5.16 (br d, J=6.4 Hz, 1H), 4.79 (br s, 1H), 4.32 (br s, 1H), 3.56-3.36 (m, 2H), 1.45 (s, 18H).

### Step B4: (S)-3,3,3-trifluoropropane-1,2-diamine dihydrochloride

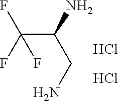

A solution of di-tert-butyl (3,3,3-trifluoropropane-1,2-diyl)(S)-dicarbamate (46 g) in HCl/dioxane (230 ml) and dioxane (230 ml) was stirred at 25° C. for 1 h. The reaction mixture concentrated under reduced pressure to give 28 g of the crude title compound with (2R)-3,3,3-trifluoropropane-1,2-diamine hydrochloride as an impurity.

### Step B5: (S)—N1,N2-dibenzyl-3,3,3-trifluoropropane-1,2-diamine

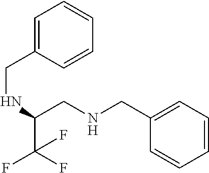 [SMILES: FC(F)(F)[C@H](CNCc1ccccc1)NCc1ccccc1]

To a solution of (S)-3,3,3-trifluoropropane-1,2-diamine dihydrochloride (8 g) in ACN (80 ml) was added K₂CO₃ (13.75 g) and benzyl bromide (13.61 g) at 0° C. The mixture was stirred at 25° C. for 2 h. The reaction mixture was filtered and concentrated under reduced pressure to give a residue, which was purified by flash silica gel chromatography (ISCO®; 80 g SepaFlash® Silica Flash Column, Eluent of 0 to 8% EA/PE gradient @60 ml/min) to give 8.6 g of a residue which was separated by SFC (column: DAICEL CHIRALPAK IG (250 mm*50 mm, 10 μm); mobile phase: [0.1% NH₃·H₂O in EtOH]; B %: 30%-30%, 8 min) to yield 6 g of the title compound. Analytical chiral analysis (Chiralpak IG-3, 4.6×50 mm, 3 μm; CO₂/EtOH (0.05% DEA); gradient: B in A from 5 to 40%; flow: 3 ml/min; T: 35° C.; back pressure: 100 bar) rt: 1.06 min. ¹H NMR (400 MHz, CDCl₃): β ppm=7.37-7.27 (m, 10H), 3.76 (d, J=13.6 Hz, 2H), 3.53 (d, J=13.6 Hz, 2H), 3.35 (tquin, J=3.7, 7.1 Hz, 1H), 2.76-2.68 (m, 1H), 2.66-2.57 (m, 1H).

### Step B6: (S)-3,3,3-trifluoropropane-1,2-diamine dihydrochloride

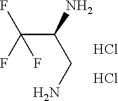

To a solution of (S)—N1,N2-dibenzyl-3,3,3-trifluoropropane-1,2-diamine (7.47 g) and HCl (12 M, 2.22 ml) in EtOH (75 ml) was added Pd/C (10%, 2.2 g) under N₂. The suspension was degassed and purged with H₂ for 3 times. The mixture was stirred under H₂ (15 psi) at 25° C. for 16 h. The reaction mixture was filtered and concentrated under reduced pressure to yield 4.88 g of the title compound. ¹H NMR (400 MHz, MeOH-d₄): δ ppm=4.62-4.48 (m, 1H), 3.63-3.56 (m, 1H), 3.52-3.44 (m, 1H).

### Step C1: Tert-butyl ((S)-2-((4-((((S)-2-amino-3,3,3-trifluoropropyl)amino)-methyl)pyridin-2-yl)amino)-1-((1r,4S)-4-methylcyclohexyl)-2-oxoethyl)carbamate

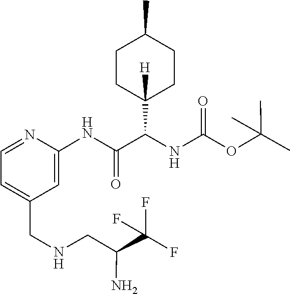

Tert-butyl ((S)-2-((4-formylpyridin-2-yl)amino)-1-((1r,4S)-4-methylcyclohexyl)-2-oxoethyl)carbamate (Step A4; 208 mg) and (S)-3,3,3-trifluoropropane-1,2-diamine dihydrochloride (Step B6; 134 mg) were dissolved in DCM (8 ml) under Ar. TEA (0.339 ml) was added and the mixture was stirred for 1.5 h at 40° C. After cooling to RT sodium cyanoborohydride (122 mg), methanol (1.2 ml), and acetic acid (0.165 ml) were added and stirring was continued for 20 min. After standing overnight the mixture was concentrated and DCM and saturated NaHCO₃ solution were added. The organic layer was separated and the aqueous phase was extracted 3× with DCM. The combined organic phases were dried over sodium sulphate, filtered and concentrated in vacuo. The residue was further purified by preparative RP HPLC (flow: 25 ml/min; gradient: 95% H₂O+0.05% TFA/5% ACN in 45 min to 5% H₂O+0.05% TFA/95% ACN; column: Purosphere® STAR-RP18, 25 mm×250 mm, 10 μm). The pure product containing fractions were combined, the ACN was partly removed, and the TFA neutralised by conc. NaHCO₃ solution. After extracting the product with DCM (3×) the combined organic phases were dried over sodium sulphate, filtered and concentrated in vacuo. The residue was dissolved in ACN/H₂O and freeze dried to yield 177 mg of the title compound (Intermediate 1). LC/MS: m/z=488.2 [M+H]+; rt: 1.71 min (LC/MS-method A).

### Intermediate 2: (S)-2-Amino-2-((1r,4S)-4-methylcyclohexyl)-N-(4-(((S)-2-oxo-4-(trifluoromethyl)imidazolidin-1-yl)methyl)pyridin-2-yl)acetamide HCl salt

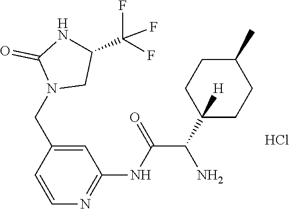

Tert-butyl ((S)-1-((1r,4S)-4-methylcyclohexyl)-2-oxo-2-((4-(((S)-2-oxo-4-(trifluoro-methyl)imidazolidin-1-yl)methyl)pyridin-2-yl)amino)ethyl)carbamate (Example 1; 127 mg) was dissolved in dioxane (3 ml) and HCl in dioxane (4 M, 3 ml) was added. After stirring for 1 h the mixture was concentrated in vacuo. The residue was dissolved in a mixture of ACN/water and freeze dried to yield 133 mg of the crude title compound. LC/MS: m/z=414.1 [M+H]⁺; rt: 1.25 min (LC/MS-method A).

### Intermediate 3: Tert-butyl ((S)-2-((4-((((S)-2-amino-3,3,3-trifluoropropyl)-amino)methyl)pyridin-2-yl)amino)-1-(4,4-difluorocyclohexyl)-2-oxoethyl)carbamate TFA Salt

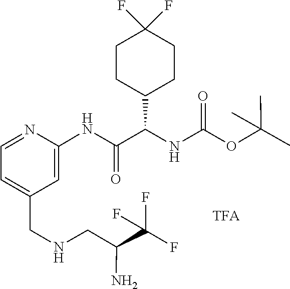 [SMILES: CC(C)(C)OC(=O)N[C@H](C(=O)Nc1cc(CNC[C@H](N)C(F)(F)F)ccn1)C1CCC(F)(F)CC1]

### Step 1: Tert-butyl (2-((4-(((tert-butyldimethylsilyl)oxy)methyl)pyridin-2-yl)amino)-1-(4,4-difluorocyclohexyl)-2-oxoethyl)carbamate

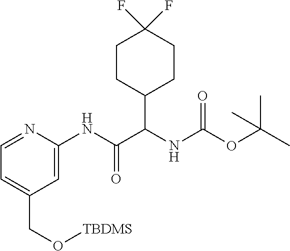

To a solution of 2-(tert-butoxycarbonylamino)-2-(4,4-difluorocyclohexyl)acetic acid (10.00 g) in DCM (100 ml) were added DMAP (8.33 g) and EDCl (13.07 g) at 20° C. over 5 min with stirring, then 4-[[tert-butyl(dimethyl)silyl]oxymethyl]pyridin-2-amine (Intermediate 1—Step A1; 8.13 g) was added and stirring was continued at 20° C. for 12 h. Then the reaction mixture was diluted with H₂O (300 ml) and extracted with DCM (200 ml×2), the combined organic layers were washed with brine (200 ml), dried over anhydrous Na₂SO₄, filtered and concentrated under reduced pressure to give a residue, which was purified by column chromatography (silica gel, PE/EA=I/O to 3/1) to yield 17 g of the title compound. ¹H NMR (400 MHz, DMSO-de): δ ppm=10.42 (s, 1H), 8.25 (d, J=5.0 Hz, 1H), 8.10 (s, 1H), 7.11 (br d, J=8.5 Hz, 1H), 7.02 (d, J=5.1 Hz, 1H), 4.74 (s, 2H), 4.18 (br t, J=7.9 Hz, 1H), 2.00 (br s, 1H), 1.84-1.56 (m, 6H), 1.38 (s, 9H), 1.29 (br s, 2H), 0.91 (s, 9H), 0.09 (s, 6H).

### Step 2: Tert-butyl (S)-(1-(4,4-difluorocyclohexyl)-2-((4-(hydroxymethyl)pyridin-2-yl)amino)-2-oxoethyl)carbamate

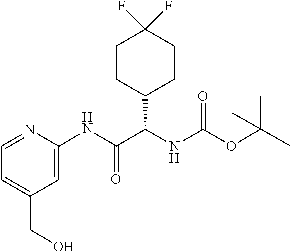 [SMILES: CC(C)(C)OC(=O)N[C@H](C(=O)Nc1cc(CO)ccn1)C1CCC(F)(F)CC1]

To a mixture of tert-butyl (2-((4-(((tert-butyldimethylsilyl)oxy)methyl)pyridin-2-yl)amino)-1-(4,4-difluorocyclohexyl)-2-oxoethyl)carbamate (17 g) in THE (173 ml) was added TBAF (1 M, 34.75 ml) in one portion at 0° C. under N₂. The mixture was stirred at 20° C. for 1 h. Then the reaction mixture was concentrated under reduced pressure. The residue was purified by column chromatography (Silica gel, PE/EA=1/0 to 2/1) to yield 11.6 g of the title compound as racemate. The racemate was separated by SFC (column: Daicel Chiralpak AD (250 mm×50 mm, 10 μm); mobile phase: [0.1% NH₃/H₂O in IPA]; B %: 50%-50%, 4 min; 180 ml/min) to yield 5.6 g of the title compound. ¹H NMR (400 MHz, CDCl₃): δ ppm=9.14-8.92 (m, 1H), 8.26 (d, J=5.1 Hz, 1H), 8.20 (s, 1H), 7.12 (d, J=5.0 Hz, 1H), 5.55 (br d, J=7.9 Hz, 1H), 4.75 (s, 2H), 4.33 (br s, 1H), 2.17-1.94 (m, 4H), 1.78 (br d, J=11.5 Hz, 3H), 1.74-1.62 (m, 2H), 1.44 (s, 9H). Analytical chiral analysis (column: Chiralpak AD-3, 4.6 mm×50 mm, 3 μm; eluents: CO₂/IPA (0.05% DEA); gradient: B in A from 5 to 40%; flow: 3 ml/min; temp: 35° C.; back pressure: 100 bar): rt: 1.64 min [for comparison: (R) enantiomer: rt: 2.70 min].

### Step 3: Tert-butyl (S)-(1-(4,4-difluorocyclohexyl)-2-((4-formylpyridin-2-yl)amino)-2-oxoethyl)carbamate

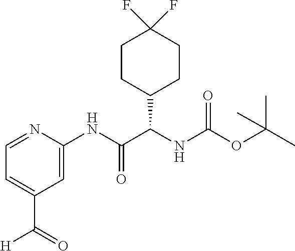 [SMILES: CC(C)(C)OC(=O)N[C@H](C(=O)Nc1cc(C=O)ccn1)C1CCC(F)(F)CC1]

A solution of tert-butyl (S)-(1-(4,4-difluorocyclohexyl)-2-((4-(hydroxymethyl)-pyridin-2-yl)amino)-2-oxoethyl)carbamate (5.6 g) in DCM (56 ml) was added DMP (8.92 g) under N₂ at 0° C. The mixture was stirred at 25° C. for 2 h. Then the reaction mixture was diluted with H₂O (200 ml) and extracted with DCM (100 ml×2), the combined organic layers were washed with brine (100 ml), dried over anhydrous Na₂SO₄, filtered and concentrated under reduced pressure to give a residue, which was purified by column chromatography (silica gel, PE/EA=1/0 to 3/1) to yield 5.3 g of the title compound. ¹H NMR (400 MHz, DMSO-de): δ ppm=10.82 (s, 1H), 10.06 (s, 1H), 8.60 (d, J=4.9 Hz, 1H), 8.51 (s, 1H), 7.54 (dd, J=1.3, 4.9 Hz, 1H), 7.16 (br d, J=8.1 Hz, 1H), 4.22 (br t, J=7.8 Hz, 1H), 2.07-2.00 (m, 2H), 1.85-1.59 (m, 6H), 1.38 (s, 9H), 1.30 (br s, 1H).

### Step 4: Tert-butyl ((S)-2-((4-((((S)-2-amino-3,3,3-trifluoropropyl)-amino)methyl)-pyridin-2-yl)amino)-1-(4,4-difluorocyclohexyl)-2-oxoethyl)carbamate TFA salt

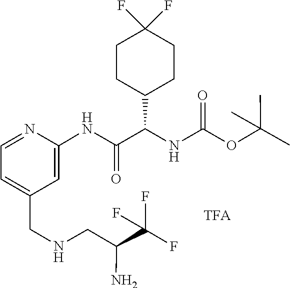 [SMILES: CC(C)(C)OC(=O)N[C@H](C(=O)Nc1cc(CNC[C@H](N)C(F)(F)F)ccn1)C1CCC(F)(F)CC1]

Tert-butyl (S)-(1-(4,4-difluorocyclohexyl)-2-((4-formylpyridin-2-yl)amino)-2-oxo-ethyl)carbamate (Step 3; 500 mg) and (2S)-3,3,3-trifluoropropane-1,2-diamine dihydrochloride salt (303 mg) were dissolved in DCM (30 ml) under Ar. TEA (0.772 ml) was added and the mixture was stirred for 2 h at 40° C. After cooling to RT sodium cyanoborohydride (277 mg), methanol (0.127 ml), and acetic acid (0.2 ml) were added and stirring was continued for 15 min. The mixture was concentrated and saturated NaHCO₃ solution and DCM were added. The organic layer was separated and the aqueous phase was extracted 3× with DCM. The combined organic phases were dried over sodium sulphate, filtered and concentrated in vacuo. The residue was further purified by preparative RP HPLC (flow: 25 ml/min; gradient: 95% H₂O+0.05% TFA/5% ACN in 45 min to 5% H₂O+0.05% TFA/95% ACN; column: Purosphere® STAR-RP18, 25 mm×250 mm, 10 μm). The pure product containing fractions were combined, the ACN was partly removed and the residue was freeze dried to yield 553 mg of the title compound (Intermediate 3). LC/MS: m/z=510.2 [M+H]⁺; rt: 1.47 min (LC/MS-method A).

### Intermediate 4: (S)-2-Amino-2-(4,4-difluorocyclohexyl)-N-(4-(((S)-2-oxo-4-(trifluoromethyl)imidazolidin-1-yl)methyl)pyridin-2-yl)acetamide HCl salt

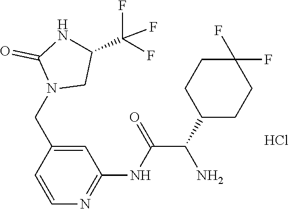 [SMILES: N[C@H](C(=O)Nc1cc(CN2C[C@@H](C(F)(F)F)NC2=O)ccn1)C1CCC(F)(F)CC1 ; Cl]

Tert-butyl N-[(1S)-1-(4,4-difluorocyclohexyl)-2-oxo-2-[[4-[[(4S)-2-oxo-4-(trifluoro-methyl)imidazolidin-1-yl]methyl]-2-pyridyl]amino]ethyl]carbamate (Example 6; 280 mg) was mixed with HCl in dioxane (4 M, 6.5 ml). After stirring for 2 h, the mixture was concentrated in vacuo. The residue was dissolved in a mixture of ACN/water and freeze dried to yield 283 mg of the crude title compound. LC/MS: m/z=436.2 [M+H]⁺; rt: 1.06 min (LC/MS-method A)

### Intermediate 5: Tert-butyl ((S)-1-((4-((((S)-2-amino-3,3,3-trifluoropropyl)amino)-methyl)pyridin-2-yl)amino)-3,3-dicyclopropyl-1-oxopropan-2-yl)carbamate TFA salt

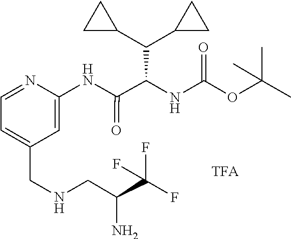 [SMILES: CC(C)(C)OC(=O)N[C@H](C(=O)Nc1cc(CNC[C@H](N)C(F)(F)F)ccn1)C(C1CC1)C1CC1]

### Step 1: Tert-butyl (1-((4-(((tert-butyldimethylsilyl)oxy)methyl)pyridin-2-yl)amino)-3,3-dicyclopropyl-1-oxopropan-2-yl)carbamate

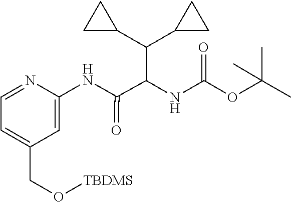

To a solution of 2-(tert-butoxycarbonylamino)-3,3-dicyclopropyl-propanoic acid (10.00 g) in DCM (100 ml) were added DMAP (9.07 g) and EDCl (14.24 g). Then 4-(((tert-butyldimethylsilyl)oxy)methyl)pyridin-2-amine (Intermediate 1—Step A1; 8.85 g) was added and stirring was continued at 20° C. for 12 h. Then the reaction mixture was diluted with H₂O (400 ml) and extracted with DCM (400 ml×2), the combined organic layers were washed with brine (200 ml), dried over anhydrous Na₂SO₄, filtered and concentrated under reduced pressure to give a residue, which was purified by column chromatography (Silica gel, PE/EA=1/0 to 73/23) to yield 13.17 g of the title compound. LC/MS: m/z=490.5 [M+H]⁺; rt: 0.55 min (LC/MS-method C).

### Step 2: Tert-butyl (S)-(1,1-dicyclopropyl-3-((4-(hydroxymethyl)pyridin-2-yl)amino)-3-oxopropan-2-yl)carbamate

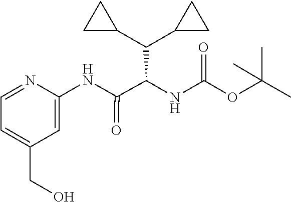 [SMILES: CC(C)(C)OC(=O)N[C@H](C(=O)Nc1cc(CO)ccn1)C(C1CC1)C1CC1]

To a mixture of tert-butyl (1-((4-(((tert-butyldimethylsilyl)oxy)methyl)pyridin-2-yl)amino)-3,3-dicyclopropyl-1-oxopropan-2-yl)carbamate (13 g) in THE (130 ml) was added TBAF (1 M, 27.87 ml) at 0° C. under N₂ with stirring The mixture was stirred at 20° C. for 1 h. Then the reaction mixture was concentrated under reduced pressure. The residue was purified by column chromatography (Silica gel, DCM/MeOH=50/1 to 10/1) to yield 9.2 g of the title compound as racemate. The racemate was separated by SFC (column: Phenomenex-Cellulose-2 (250 mm×30 mm, 10 μm); mobile phase: [0.1% NH₃/H₂O in MeOH]; B %: 20%-20%, 2.3 min; 60 ml/min) to yield 3.0 g of the title compound. ¹H NMR (400 MHz, DMSO-d₆, representative signals shown): δ ppm=10.25 (s, 1H), 8.21 (d, J=5.1 Hz, 1H), 8.05 (s, 1H), 7.02 (dd, J=0.6, 5.0 Hz, 1H), 6.91 (br d, J=8.6 Hz, 1H), 5.41 (t, J=5.8 Hz, 1H), 4.52 (d, J=5.8 Hz, 2H), 4.41-4.35 (m, 1H), 1.40 (s, 9H). Analytical chiral analysis (column: Chiralcel OZ-3, 4.6×50 mm, 3 μm; eluents: CO₂/MeOH (0.05% DEA); gradient: B in A from 5 to 40%; flow: 3 ml/min; T: 35° C.; back pressure: 100 bar): rt: 0.817 min [for comparison: (R) enantiomer: rt: 0.988 min].

### Step 3: Tert-butyl (S)-(1,1-dicyclopropyl-3-((4-formylpyridin-2-yl)amino)-3-oxopropan-2-yl)carbamate

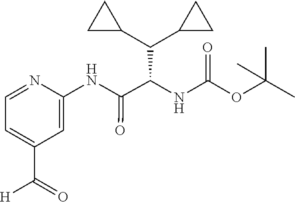 [SMILES: CC(C)(C)OC(=O)N[C@H](C(=O)Nc1cc(C=O)ccn1)C(C1CC1)C1CC1]

A solution of tert-butyl (S)-(1,1-dicyclopropyl-3-((4-(hydroxymethyl)pyridin-2-yl)amino)-3-oxopropan-2-yl)carbamate (2.9 g) in DCM (30 ml) was added DMP (4.91 g) with stirring at 0° C. The mixture was stirred at 20° C. for 2 h. Then the mixture was filtered and the filtrate was diluted with H₂O (100 ml) and extracted with EA (100 ml×3), the combined organic layers were washed with brine (100 ml), dried over anhydrous Na₂SO₄, filtered and concentrated under reduced pressure to give a residue, which was purified by column chromatography (Silica gel, PE/EA=1/0 to 3/1) to yield 2.79 g of the title compound. ¹H NMR (400 MHz, DMSO-d₆, representative signals shown): δ ppm=10.53 (s, 1H), 9.87 (s, 1H), 8.40 (d, J=4.9 Hz, 1H), 8.29 (s, 1H), 7.34 (dd, J=0.9, 5.0 Hz, 1H), 6.79 (br d, J=8.6 Hz, 1H), 4.31-4.14 (m, 1H), 1.26-1.12 (m, 10H).

### Step 4: Tert-butyl ((S)-1-((4-((((S)-2-amino-3,3,3-trifluoropropyl)amino)-methyl)pyridin-2-yl)amino)-3,3-dicyclopropyl-1-oxopropan-2-yl)carbamate TFA salt

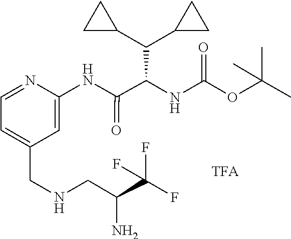 [SMILES: CC(C)(C)OC(=O)N[C@H](C(=O)Nc1cc(CNC[C@H](N)C(F)(F)F)ccn1)C(C1CC1)C1CC1]

Tert-butyl (S)-(1,1-dicyclopropyl-3-((4-formylpyridin-2-yl)amino)-3-oxopropan-2-yl)carbamate (500 mg) and (2S)-3,3,3-trifluoropropane-1,2-diamine dihydrochloride salt (323 mg) were dissolved in DCM (30 ml) under Ar. After the addition of TEA (0.821 ml) the mixture was stirred for 10 min at RT and then for 1.5 h at 40° C. After cooling to RT sodium cyanoborohydride (294 mg), methanol (7 ml), and acetic acid (0.399 ml) were added, and stirring was continued for 1 h. The mixture was concentrated and saturated NaHCO₃ solution and DCM were added. The organic layer was separated and the aqueous phase was extracted with DCM (3×). The combined organic phases were dried over sodium sulphate, filtered and concentrated in vacuo. The residue was further purified by preparative RP HPLC (flow: 25 ml/min; gradient: 95% H₂O+0.05% TFA/5% ACN in 45 min to 5% H₂O+0.05% TFA/95% ACN; column: Purosphere® STAR-RP18, 25 mm×250 mm, 10 μm). The pure product containing fractions were combined, the ACN was partly removed and the residue was freeze dried to yield 603 mg of the title compound (Intermediate 5). LC/MS: m/z=486.2 [M+H]⁺; rt: 1.57 min (LC/MS-method A)

### Intermediate 6: (S)-2-Amino-3,3-dicyclopropyl-N-(4-(((S)-2-oxo-4-(trifluoromethyl)-imidazolidin-1-yl)methyl)pyridin-2-yl)propanamide HCl salt

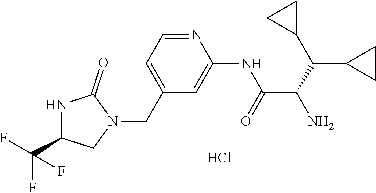 [SMILES: N[C@H](C(=O)Nc1cc(CN2C[C@@H](C(F)(F)F)NC2=O)ccn1)C(C1CC1)C1CC1 ; Cl]

Analogously to Intermediate 4, 323 mg of the crude title compound were obtained. LC/MS: m/z=412.2 [M+H]⁺; rt: 1.13 min (LC/MS-method A)

### Intermediate 7: Tert-butyl ((S)-2-((4-((((1-(methylamino)cyclobutyl)methyl)amino)-methyl)pyridin-2-yl)amino)-1-((1r,4S)-4-methylcyclohexyl)-2-oxoethyl)carbamate

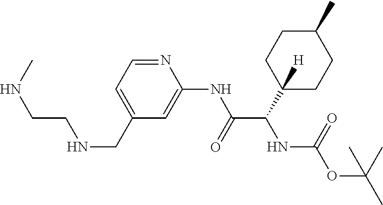

Tert-butyl ((S)-2-((4-formylpyridin-2-yl)amino)-1-((1r,4S)-4-methylcyclohexyl)-2-oxoethyl)carbamate [Intermediate 1—Step A4] (30 mg) and 1-(aminomethyl)-N-methyl-cyclobutanamine dihydrochloride salt (37 mg) were dissolved in DCM (2 ml) under Ar. After the addition of TEA (0.105 ml) the mixture was stirred for 10 min at RT and then for 1 h at 40° C. After cooling to RT sodium cyanoborohydride (18 mg), methanol (2.5 ml) and acetic acid (57 μl) were added and stirring was continued for 1 h. After standing overnight the mixture was concentrated and saturated NaHCO₃ solution and DCM were added. The organic layer was separated and the aqueous phase was extracted with DCM (3×). The combined organic phases were dried over sodium sulphate, filtered and concentrated in vacuo. The residue was further purified by preparative RP HPLC (flow: 25 ml/min; gradient: 95% H₂O+0.05% TFA/5% ACN in 45 min to 5% H₂O+0.05% TFA/95% ACN; column: Purosphere® STAR-RP18, 25 mm×250 mm, 10 μm). The pure product containing fractions were combined, the ACN was removed, saturated NaHCO₃ solution was added (pH~7) and the aqueous mixture was extracted with DCM (2×). The combined organic phases were dried over sodium sulphate, filtered and concentrated in vacuo to yield 22 mg of the title compound. LC/MS: m/z=474.3 [M+H]⁺; rt: 0.73 min (LC/MS-method B).

### Intermediate 8: Tert-butyl ((S)-2-((4-(((2-amino-2-methylpropyl)amino)methyl)-pyridin-2-yl)amino)-1-((1r,4S)-4-methylcyclohexyl)-2-oxoethyl)carbamate

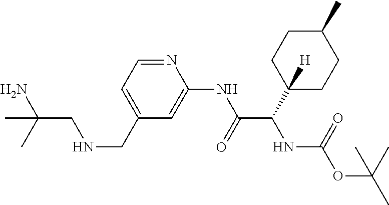

### Step 1: Tert-butyl ((S)-2-((4-(((2-(((benzyloxy)carbonyl)amino)-2-methylpropyl)amino)-methyl)pyridin-2-yl)amino)-1-((1r,4S)-4-methylcyclohexyl)-2-oxoethyl)carbamate

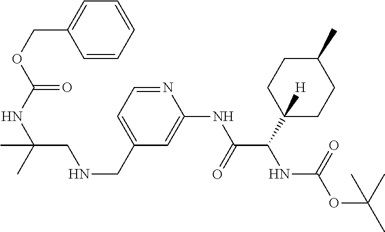

Tert-butyl ((S)-2-((4-formylpyridin-2-yl)amino)-1-((1r,4S)-4-methylcyclohexyl)-2-oxoethyl)carbamate [Intermediate 1—Step A4] (100 mg) and benzyl N-(2-amino-1,1-dimethyl-ethyl)carbamate hydrochloride salt (181 mg) were dissolved in DCM (2 ml) under Ar. After the addition of TEA (0.163 ml), the mixture was stirred for 10 min at RT and then for 1 h at 40° C. After cooling to RT sodium cyanoborohydride (59 mg), methanol (4.5 ml) and acetic acid (79 μl) were added and stirring was continued for 1 h. Then the mixture was concentrated and DCM and saturated NaHCO₃ solution were added. The organic layer was separated and the aqueous phase was extracted with DCM (2×). The combined organic phases were dried over sodium sulphate, filtered and concentrated in vacuo. The residue was further purified by preparative RP HPLC (Agilent Prep C18, 30 mm×250 mm, 10 μm; 50 ml/min, from 90% H₂O/10% ACN to 10% H₂O/90% ACN in 12.5 min). The pure product containing fractions were combined, the ACN was partly removed and the residue freeze dried to yield 63 mg of the compound. LC/MS: m/z=582.2 [M+H]⁺; rt: 1.97 min (LC/MS-method A).

### Step 2: Tert-butyl ((S)-2-((4-(((2-amino-2-methylpropyl)amino)methyl)pyridin-2-yl)amino)-1-((1r,4S)-4-methylcyclohexyl)-2-oxoethyl)carbamate

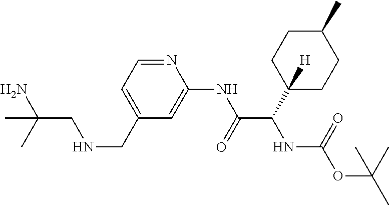

Tert-butyl ((S)-2-((4-(((2-(((benzyloxy)carbonyl)amino)-2-methylpropyl)amino)-methyl)pyridin-2-yl)amino)-1-((1r,4S)-4-methylcyclohexyl)-2-oxoethyl)carbamate (62 mg) was dissolved in MeOH (2.5 ml) and Pd/C (10%, 6 mg) was added. After flushing with hydrogen the mixture was stirred for 20 h under a hydrogen atmosphere (balloon). To complete the reaction, the mixture was filtered and washed with MeOH. The volume of the filtrate was reduced in vacuo, Pd/C (6 mg) was added and hydrogen was bubbled through the mixture for 1 h. Then the mixture was filtered, washed with MeOH and the filtrated was concentrated in vacuo. The residue was further purified by preparative RP HPLC (flow: 25 ml/min; gradient: 95% H2O+0.05% TFA/5% ACN in 45 min to 5% H2O+0.05% TFA/95% ACN; column: Purosphere® STAR-RP18, 25 mm×250 mm, 10 μm). The pure product containing fractions were combined, the ACN was removed, saturated NaHCO₃ solution was added (pH~7) and the aqueous mixture was extracted with DCM (2×). The combined organic phases were dried over sodium sulphate, filtered and concentrated in vacuo to yield 20 mg of the title compound (Intermediate 8). LC/MS: m/z=448.3 [M+H]⁺; rt: 0.711 min (LC/MS-method B).

### Intermediate 9: (S)-2-Amino-N-(4-((4,4-dimethyl-2-oxoimidazolidin-1-yl)methyl)pyridin-2-yl)-2-((1r,4S)-4-methylcyclohexyl)acetamide HCl salt

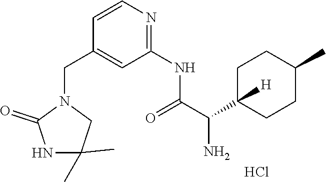

Analogously to Intermediate 2, 13 mg of the crude title compound was obtained. LC/MS: m/z=374.2 [M+H]⁺; rt: 1.27 min (LC/MS-method A).

### Intermediate 10: (2-Aminopyridin-4-yl)methyl (2,2,2-trifluoroethyl)carbamate

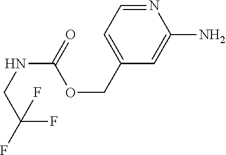 [SMILES: Nc1cc(COC(=O)NCC(F)(F)F)ccn1]

### Step 1: Tert-butyl (4-(hydroxymethyl)pyridin-2-yl)carbamate

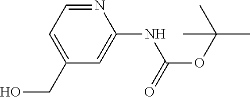 [SMILES: CC(C)(C)OC(=O)Nc1cc(CO)ccn1]

Tert-butyl N-(4-formyl-2-pyridyl)carbamate (100 mg) was dissolved in MeOH (5 ml) under Ar. Sodium borohydride (19 mg) was added with stirring. After stirring for 1 h and standing overnight, the MeOH was reduced and the residue treated with EA. The mixture was poured into water and the aqueous mixture was extracted with EA (2×). The combined organic phases were dried over sodium sulphate, filtered and concentrated in vacuo. The residue was purified by silica gel chromatography (DCM/EtOH: 100:0 for 5 min, 100:0 to 95:5 in 30 min, 95:5 for 10 min). The pure compound containing fractions were combined and concentrated in vacuo to yield 82 mg of the title compound. LC/MS: m/z=225.1 [M+H]⁺; rt: 0.380 min (LC/MS-method B).

### Step 2: Tert-butyl (4-((((2,2,2-trifluoroethyl)carbamoyl)oxy)methyl)pyridin-2-yl)carbamate

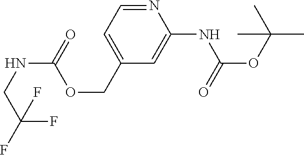 [SMILES: CC(C)(C)OC(=O)Nc1cc(COC(=O)NCC(F)(F)F)ccn1]

Tert-butyl (4-(hydroxymethyl)pyridin-2-yl)carbamate (97 mg) was dissolved in THE (4 ml) and NaH (60% in mineral oil, 52 mg) was added. After stirring for 1 h, 1,1,1-trifluoro-2-isocyanato-ethane (136 mg) was added. After 6 h an additional equivalent of 1,1,1-trifluoro-2-isocyanato-ethane was added. After stirring overnight the mixture was poured into water and the aqueous mixture was extracted with EA (2×). The combined organic phases were dried over sodium sulphate, filtered and concentrated in vacuo. The residue was purified by preparative RP HPLC (Agilent Prep C18, 30 mm×250 mm, 10 μm; 40 ml/min, from 90% H₂O/10% ACN to 10% H₂O/90% ACN in 15 min). The pure product containing fractions were combined, the ACN was partly removed and the residue freeze dried to yield 85 mg of the title compound. LC/MS: m/z=350.1 [M+H]⁺; rt: 0.716 min (LC/MS-method B).

### Step 3: (2-Aminopyridin-4-yl)methyl (2,2,2-trifluoroethyl)carbamate

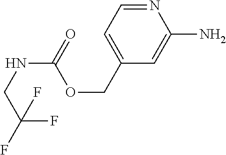 [SMILES: Nc1cc(COC(=O)NCC(F)(F)F)ccn1]

Tert-butyl (4-((((2,2,2-trifluoroethyl)carbamoyl)oxy)methyl)pyridin-2-yl)carbamate (72 mg) was dissolved in dioxane (2.5 ml) and HCl in dioxane (4 M, 2.5 ml) was added. After stirring for 2.5 h at 50° C., the mixture was cooled and concentrated in vacuo. The residue was treated with EA and water. Saturated NaHCO₃ solution was added (pH~7) and the aqueous phase was extracted with EA (2×). The combined organic phases were dried over sodium sulphate, filtered and concentrated in vacuo. The residue was purified by silica gel chromatography (DCM/EtOH: 100:0 for 5 min, 100:0 to 80:20 in 40 min, 80:20 for 10 min). The pure compound containing fractions were combined and concentrated in vacuo to yield 32 mg of the title compound (Intermediate 10). LC/MS: m/z=250.0 [M+H]⁺; rt: 0.65 min (LC/MS-method A).

### Intermediate 11: Tert-butyl (S)-(2-((4-(bromomethyl)pyridin-2-yl)amino)-1-(4,4-difluorocyclohexyl)-2-oxoethyl)carbamate

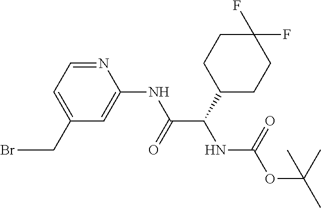 [SMILES: CC(C)(C)OC(=O)N[C@H](C(=O)Nc1cc(CBr)ccn1)C1CCC(F)(F)CC1]

### Step 1: Tert-butyl (S)-(1-(4,4-difluorocyclohexyl)-2-((4-(hydroxymethyl)pyridin-2-yl)amino)-2-oxoethyl)carbamate

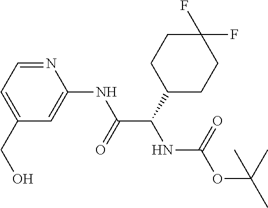 [SMILES: CC(C)(C)OC(=O)N[C@H](C(=O)Nc1cc(CO)ccn1)C1CCC(F)(F)CC1]

Tert-butyl (S)-(1-(4,4-difluorocyclohexyl)-2-((4-formylpyridin-2-yl)amino)-2-oxoethyl)carbamate [Intermediate 3—Step 3] (215 mg) was dissolved in MeOH (5 ml). Sodium borohydride (23 mg) was added with stirring. After stirring for 5 h and standing overnight, H₂O was added to the mixture and the aqueous phase was extracted with EA (3×). The combined organic phases were dried over sodium sulphate, filtered and concentrated in vacuo. The residue was purified by silica gel chromatography (Silica gel 12 g; nHep/EA: 100:0 for 5 min, 100:0 to 40:60 in 30 min). The pure compound containing fractions were combined and concentrated in vacuo to yield 207 mg of the title compound. LC/MS: m/z=400.2 [M+H]⁺; rt: 1.73 min (LC/MS-method A).

### Step 2: Tert-butyl (S)-(2-((4-(bromomethyl)pyridin-2-yl)amino)-1-(4,4-difluorocyclohexyl)-2-oxoethyl)carbamate

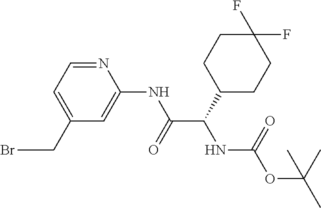 [SMILES: CC(C)(C)OC(=O)N[C@H](C(=O)Nc1cc(CBr)ccn1)C1CCC(F)(F)CC1]

Tert-butyl (S)-(1-(4,4-difluorocyclohexyl)-2-((4-(hydroxymethyl)pyridin-2-yl)amino)-2-oxoethyl)carbamate (50 mg) was dissolved in dry THE (2 ml) under Ar. Triphenylphosphine (66 mg) and carbon tetrabromide (62 mg) were added with stirring. After stirring for 2 h, the mixture was put in a deep freezer over the weekend. After warming the mixture was filtered and the filtrate concentrated in vacuo. The residue was purified by silica gel chromatography (nHep/EA: 100:0 for 5 min, 100:0 to 90:10 in 40 min). The pure compound containing fractions were combined and concentrated in vacuo to yield 19 mg of the title compound (Intermediate 11). LC/MS: m/z=462.0 [M+H]⁺; rt: 2.53 min (LC/MS-method A).

### Intermediate 12: Tert-butyl ((S)-2-((4-((((R)-2-aminopropyl)amino)methyl)pyridin-2-yl)amino)-1-((1r,4S)-4-methylcyclohexyl)-2-oxoethyl)carbamate and tert-butyl ((S)-2-((4-((((R)-1-aminopropan-2-yl)amino)methyl)pyridin-2-yl)amino)-1-((1r,4S)-4-methylcyclohexyl)-2-oxoethyl)carbamate

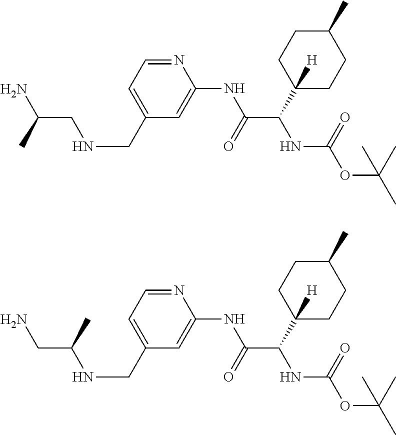

Following the procedure in Intermediate 1 using tert-butyl ((S)-2-((4-formylpyridin-2-yl)amino)-1-((1r,4S)-4-methylcyclohexyl)-2-oxoethyl)carbamate [Intermediate 1—Step A4] (30 mg) and (2R)-propane-1,2-diamine dihydrochloride salt (29 mg) as starting materials 15 mg of the title compounds were obtained as a mixture. LC/MS: m/z=434.3 [M+H]⁺; rt: 0.704 min (LC/MS-method B).

### Intermediate 13: Methyl (S)-2-((tert-butoxycarbonyl)amino)-2-(4,4-difluorocyclohexyl)acetate

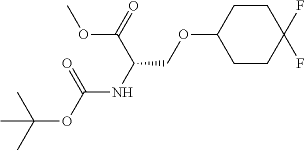 [SMILES: COC(=O)[C@H](COC1CCC(F)(F)CC1)NC(=O)OC(C)(C)C]

Under Ar (2S)-2-(tert-butoxycarbonylamino)-2-(4,4-difluorocyclohexyl)acetic acid (1 g) was dissolved in DMF (15 ml). Cs₂CO₃ (555 mg) was added followed by methyl iodide (0.23 ml). After stirring for 1 h, water was added (30 ml) and the aqueous phase was extracted with MTB ether. The combined organic phases were dried over sodium sulphate, filtered and concentrated in vacuo. The residue was purified by silica gel chromatography (Silica gel 24 g; 32 ml/min; nHep/EA 85:15 to 60:40 in 23 min). The pure compound containing fractions were combined and concentrated in vacuo to yield 1 g of the title compound. LC/MS: m/z=252.1 [M+H-isobutene]+; rt: 2.16 min (LC/MS-method A).

### Intermediate 14: Tert-butyl ((S)-2-((4-((((S)-2-amino-3,3,3-trifluoropropyl)amino)-methyl)-5-fluoropyridin-2-yl)amino)-1-((1r,4S)-4-methylcyclohexyl)-2-oxoethyl)carbamate

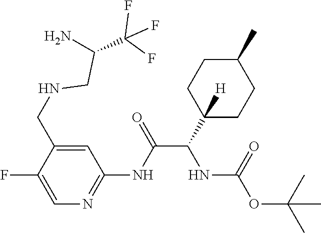

### Step 1: Methyl 2-((tert-butoxycarbonyl)amino)-5-fluoroisonicotinate

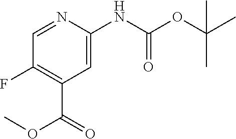 [SMILES: COC(=O)c1cc(NC(=O)OC(C)(C)C)ncc1F]

To a mixture of methyl 2-bromo-5-fluoro-pyridine-4-carboxylate (20.5 g) in dioxane (310 ml) was added tert-butyl carbamate (25.65 g), Pd₂(dba)₃ (1.60 g), Cs₂CO₃ (39.96 g) and Xantphos (2.03 g) in one portion at 20° C. under N₂. The mixture was stirred at 90° C. for 12 h. The reaction mixture was cooled, filtrated through pad of celite and washed with EA (10 ml), filtrate was dissolved into EA (200 ml), then washed with water (100 ml) and brine (100 ml), dried over anhydrous Na₂SO₄, and concentrated under reduced pressure to give the crude, which was triturated with EA (100 ml) to give 19.06 g of the title compound. ¹H NMR (400 MHz, CDCl₃): β ppm=8.41 (br d, J=5.1 Hz, 1H), 8.26 (s, 1H), 8.15 (br s, 1H), 3.96 (s, 3H), 1.56 (s, 9H).

### Step 2: Tert-butyl (5-fluoro-4-(hydroxymethyl)pyridin-2-yl)carbamate

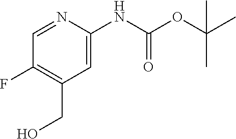 [SMILES: CC(C)(C)OC(=O)Nc1cc(CO)c(F)cn1]

To a mixture of methyl 2-((tert-butoxycarbonyl)amino)-5-fluoroisonicotinate (10 g) in THE (130 ml) was added LiBH₄ (2.02 g) in one portion at 20° C. under N₂. The mixture was stirred at 40° C. for 4 h. Aq. NaHCO₃ was added to the mixture at 0° C. and diluted with water (100 ml) and EA (150 ml), the combined organic phases were washed with brine (100 ml), dried over anhydrous Na₂SO₄ and concentrated to give the residue, which was purified by column chromatography (Silica gel, PE/EA=0/1 to 3/1) to yield 6.5 g of the title compound. ¹H NMR (400 MHz, CDCl₃): β ppm=8.10 (d, J=5.4 Hz, 1H), 8.06 (d, J=1.1 Hz, 1H), 7.80 (br s, 1H), 4.80 (s, 2H), 1.54 (s, 9H).

### Step 3: (2-Amino-5-fluoropyridin-4-yl)methanol

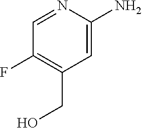

A mixture of tert-butyl (5-fluoro-4-(hydroxymethyl)pyridin-2-yl)carbamate (6.5 g) in HCl/MeOH (65 ml) was stirred at 20° C. for 12 h under N₂. The reaction mixture was concentrated under reduced pressure to yield 4.79 g of the title compound.

### Step 4: 4-(((Tert-butyldimethylsilyl)oxy)methyl)-5-fluoropyridin-2-amine

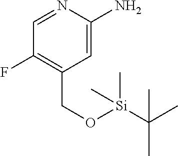 [SMILES: CC(C)(C)[Si](C)(C)OCc1cc(N)ncc1F]

To a mixture of (2-amino-5-fluoropyridin-4-yl)methanol (4.79 g) and imidazole (5.48 g) in DCM (38 ml) was added TBDMSCl (6.06 g) in one portion at 0° C. under N₂. The reaction mixture was stirred at 20° C. for 12 h. The reaction mixture was concentrated under reduced pressure to give the residue, which was purified by column chromatography (Silica gel, PE/EA=0/1 to 3/1) to yield 4.4 g of the title compound. ¹H NMR (400 MHz, CDCl₃): β ppm=7.80 (d, J=1.8 Hz, 1H), 6.65 (d, J=4.8 Hz, 1H), 4.72 (s, 2H), 0.97-0.92 (m, 9H), 0.13 (s, 6H).

### Step 5: Tert-butyl ((S)-2-((4-(((tert-butyldimethylsilyl)oxy)methyl)-5-fluoropyridin-2-yl)amino)-1-((1r,4S)-4-methylcyclohexyl)-2-oxoethyl)carbamate

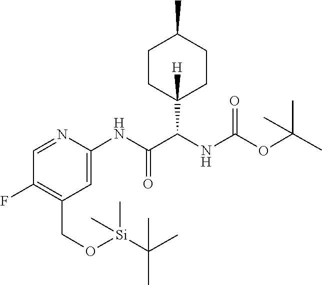

To a mixture of (2S)-2-(tert-butoxycarbonylamino)-2-(trans-4-methyl-cyclohexyl)acetic acid (5.29 g) in DCM (50 ml) was added DMAP (4.76 g) and EDC (7.48 g) at 20° C. for 5 min, then 4-(((tert-butyldimethylsilyl)oxy)methyl)-5-fluoropyridin-2-amine (5 g) was added and stirred at 20° C. for 12 h. DCM (100 ml) and water (50 ml) were added to the mixture. The aqueous phase was extracted with DCM (2×). The combined organic phases were, dried over anhydrous Na₂SO₄, filtered, and concentrated in vacuo. The residue was purified by column chromatography (Silica gel, PE/EA=0/1 to 3/1) to yield 9.12 g of the title compound.

### Step 6: Tert-butyl ((S)-2-((5-fluoro-4-(hydroxymethyl)pyridin-2-yl)amino)-1-((1r,4S)-4-methylcyclohexyl)-2-oxoethyl)carbamate

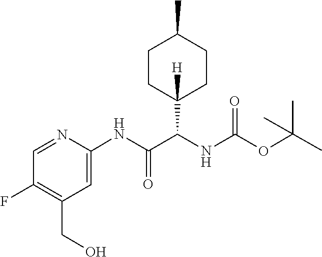

To a mixture of tert-butyl ((S)-2-((4-(((tert-butyldimethylsilyl)oxy)methyl)-5-fluoropyridin-2-yl)amino)-1-((1r,4S)-4-methylcyclohexyl)-2-oxoethyl)carbamate (9.12 g) in THE (91 ml) was added TBAF (1 M, 18.79 ml) in one portion at 0° C. under N₂. The reaction mixture was stirred at 20° C. for 12 h. The reaction mixture was concentrated under reduced pressure. The residue was purified by column chromatography (Silica gel, PE/EA=0/1 to 3/1). Additional SFC purification (Daicel chiralcel OJ (250 mm×30 mm, 10 μm); [0.1% NH₃/H₂O in MeOH]; B %: 10%-10%, 2 min; 200 ml/min) yielded 3.4 g of the title compound (peak 1). Analytical SFC data (Daicel chiralcel OJ-3 (50 mm×4.6 mm, 3 μm); MeOH (0.05% DEA)]; B %: 5%-40%, flow 3 ml/min, T: 35° C.): peak 1: RT=0.54 min; peak 2: RT=0.69 min.

### Step 7: Tert-butyl ((S)-2-((5-fluoro-4-formylpyridin-2-yl)amino)-1-((1r,4S)-4-methylcyclohexyl)-2-oxoethyl)carbamate

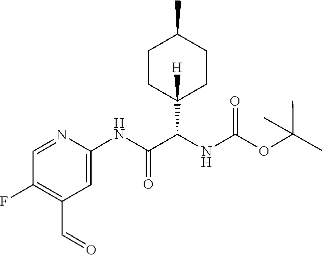

To a mixture of tert-butyl ((S)-2-((5-fluoro-4-(hydroxymethyl)pyridin-2-yl)amino)-1-((1r,4S)-4-methylcyclohexyl)-2-oxoethyl)carbamate (3.7 g) in DCM (37 ml) was added DMP (5.95 g) in one portion at 0° C. under N₂. The mixture was stirred at 20° C. for 2 h. The reaction mixture was filtered and the filtrate was concentrated under reduced pressure to give a residue, which was purified by column chromatography (Silica gel, PE/EA=0/1 to 1/1) to yield 3.1 g of the title compound. ¹H NMR (400 MHz, CDCl₃): β ppm=10.33 (s, 1H), 8.71 (br s, 1H), 8.59 (d, J=5.0 Hz, 1H), 8.34 (s, 1H), 5.11 (br d, J=8.3 Hz, 1H), 4.13 (br d, J=6.9 Hz, 1H), 1.85-1.72 (m, 5H), 1.46 (s, 9H), 1.36-1.26 (m, 1H), 1.23-1.04 (m, 2H), 1.02-0.91 (m, 2H), 0.88 (d, J=6.5 Hz, 3H).

### Step 8: Tert-butyl ((S)-2-((4-((((S)-2-amino-3,3,3-trifluoropropyl)amino)methyl)-5-fluoropyridin-2-yl)amino)-1-((1r,4S)-4-methylcyclohexyl)-2-oxoethyl)carbamate

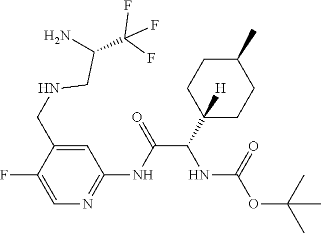

Tert-butyl ((S)-2-((5-fluoro-4-formylpyridin-2-yl)amino)-1-((1r,4S)-4-methylcyclo-hexyl)-2-oxoethyl)-carbamate (500 mg) and (2S)-3,3,3-trifluoropropane-1,2-diamine dihydrochloride salt [Intermediate 1—Step B6] (307 mg) were dissolved in DCM (30 ml) under Ar. TEA (0.779 ml) was added and the mixture was stirred for 40 min at 40° C. After cooling to RT, sodium cyanoborohydride (280 mg), dry methanol (0.129 ml) and acetic acid (0.4 ml) were added, and stirring was continued for 20 min. The mixture was concentrated and DCM and saturated NaHCO₃ solution were added. The organic layer was separated and the aqueous phase was extracted 3× with DCM. The combined organic phases were dried over sodium sulphate, filtered and concentrated in vacuo. The residue was further purified by preparative RP HPLC (flow: 25 ml/min; gradient: 95% H₂O+0.05% TFA/5% ACN in 45 min to 5% H₂O+0.05% TFA/95% ACN; column: Purosphere® STAR-RP18, 25 mm×250 mm, 10 μm). The pure product containing fractions were combined, the ACN was partly removed and the TFA neutralised by conc. NaHCO₃ solution. After extracting the product with DCM (3×), the combined organic phases were dried over sodium sulphate, filtered and concentrated in vacuo. The residue was dissolved in ACN/H₂O and freeze dried to yield 412 mg of the title compound (Intermediate 14). LC/MS: m/z=506.3 [M+H]⁺; rt: 1.79 min (LC/MS-method A).

### Intermediate 15: (S)-1-((2-Aminopyridin-4-yl)methyl)-4-(trifluoromethyl)-imidazolidin-2-one

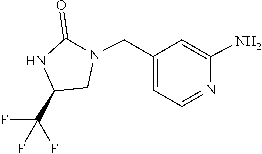 [SMILES: Nc1cc(CN2C[C@@H](C(F)(F)F)NC2=O)ccn1]

### Step 1: Tert-butyl (S)-(4-(((2-amino-3,3,3-trifluoropropyl)amino)methyl)pyridin-2-yl)carbamate

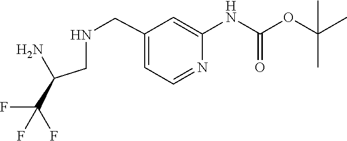 [SMILES: CC(C)(C)OC(=O)Nc1cc(CNC[C@H](N)C(F)(F)F)ccn1]

Following the procedure of Intermediate 14, using tert-butyl (4-formylpyridin-2-yl)carbamate (750 mg), 794 mg of the title compound was obtained. LC/MS: m/z=335.2 [M+H]⁺; rt: 1.08 min (LC/MS-method A).

### Step 2: Tert-butyl (S)-(4-((2-oxo-4-(trifluoromethyl)imidazolidin-1-yl)methyl)pyridin-2-yl)carbamate

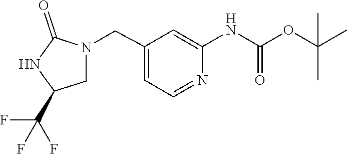 [SMILES: CC(C)(C)OC(=O)Nc1cc(CN2C[C@@H](C(F)(F)F)NC2=O)ccn1]

Tert-butyl (S)-(4-(((2-amino-3,3,3-trifluoropropyl)amino)methyl)pyridin-2-yl)carbamate (435 mg) was dissolved in THE (12 ml). After the addition of DIPEA (0.906 ml) the mixture was heated to 65° C. for 5 min. Then CDI (633 mg) was added and stirring was continued overnight at 65° C. At RT, some of the THF was distilled off and MeOH (5 ml) and aqueous NaOH solution (2 N, 1 ml) were added. After 15 min the mixture was poured onto saturated NaHCO₃ solution and DCM was added. The organic layer was separated and the aqueous phase was extracted twice with DCM. The combined organic phases were dried over sodium sulphate, filtered and concentrated in vacuo. The residue was purified by silica gel column chromatography (12 g silica gel; gradient: DCM/EtOH: 100% DCM for 5 min, 100% to 95% DCM in 60 min; 95% DCM for 10 min) and the combined product containing fractions were concentrated in vacuo to yield 410 mg of the title compound. LC/MS: m/z=361.2 [M+H]⁺; rt: 1.56 min (LC/MS-method A).

### Step 3: (S)-1-((2-Aminopyridin-4-yl)methyl)-4-(trifluoromethyl)imidazolidin-2-one

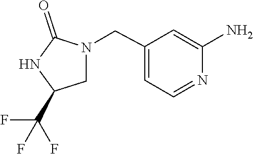 [SMILES: Nc1cc(CN2C[C@@H](C(F)(F)F)NC2=O)ccn1]

Following the procedure of Intermediate 10,398 mg of the crude title compound (Intermediate 15) was obtained which were used without further purification. LC/MS: m/z=261.0 [M+H]⁺; rt: 0.4 min (LC/MS-method A).

### Intermediate 16: (S)-2-(4,4-Difluorocyclohexyl)-2-((((5-methylisoxazol-3-yl)methoxy)carbonyl)amino)acetic acid

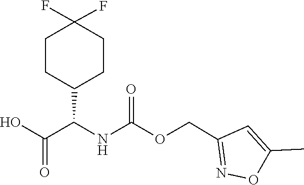 [SMILES: Cc1cc(COC(=O)N[C@H](C(=O)O)C2CCC(F)(F)CC2)no1]

### Step 1: Methyl (S)-2-amino-2-(4,4-difluorocyclohexyl)acetate

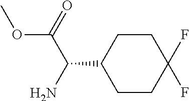

Methyl (S)-2-((tert-butoxycarbonyl)amino)-2-(4,4-difluorocyclohexyl)acetate (Intermediate 13, 100 mg) was dissolved in dioxane (1.5 ml) and HCl in dioxane (4 M, 1.5 ml), was added. After stirring for 5 h at RT the mixture stood overnight. The mixture was concentrated in vacuo and DCM and saturated NaHCO₃ solution were added (pH~7). The aqueous phase was extracted with DCM (2×). The combined organic phases were dried over sodium sulphate, filtered and concentrated in vacuo to yield 63 mg of the crude title compound, which was used directly in the next step. LC/MS: m/z=208.1 [M+H]⁺; rt: 0.31 min (LC/MS-method B).

### Step 2: Methyl (S)-2-(4,4-difluorocyclohexyl)-2-((((5-methylisoxazol-3-yl)methoxy)-carbonyl)amino)-acetate

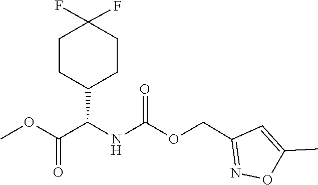

(4-Nitrophenyl) carbonochloridate (80 mg) was dissolved in dry THE (2 ml) under Ar. After cooling to −78° C. methyl (S)-2-amino-2-(4,4-difluorocyclohexyl)acetate (79 mg, dissolved in dry THE (2 ml)) and DIPEA (99 μl) were added with stirring. After 2 h, NaH (40 mg, 60% in mineral oil) was mixed with dry THE (3 ml) under Ar. Then (5-methylisoxazol-3-yl)methanol dissolved in dry THE (0.5 ml) was added with stirring. After 30 min, this mixture was dropwise added to the mixture above at −78° C. After 5 min, the cooling bath was removed, and 1 h later the mixture was poured onto saturated Na₂CO₃ solution. DCM was added and the organic phase was washed with saturated Na₂CO₃ (4×) and finally saturated NaHCO₃ solution. The organic phase was dried over sodium sulphate, filtered and concentrates in vacuo. The residue was purified by preparative RP HPLC (Purosphere® STAR-RP18, 25 mm×250 mm, 10 μm; 30 ml/min, from 95% H₂O/5% ACN to 10% H₂O/90% ACN in 42 min; then 10% H₂O/90% ACN for 8 min and 100% ACN for 3 min). The pure product containing fractions were combined, the ACN was partly removed and the residue freeze dried to yield 64 mg of the title compound. LC/MS: m/z=347.2 [M+H]⁺; rt: 1.88 min (LC/MS-method A).

### Step 3: (S)-2-(4,4-Difluorocyclohexyl)-2-((((5-methylisoxazol-3-yl)methoxy)carbonyl)amino)acetic acid

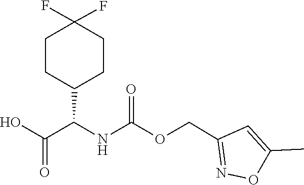 [SMILES: Cc1cc(COC(=O)N[C@H](C(=O)O)C2CCC(F)(F)CC2)no1]

Methyl (S)-2-(4,4-difluorocyclohexyl)-2-((((5-methylisoxazol-3-yl)methoxy)-carbonyl)amino)acetate (63 mg) was dissolved in THE/water (1.6/0.4 ml) and LiOH (9 mg) was added with stirring. After 2 h, aqueous HCl was added (1 M) to adjust the pH to 2-3. Then the aqueous phase was extracted with EA (2×). The combined organic phases were dried (Chem Elut cartridge) and concentrated in vacuo. The residue was purified by preparative RP HPLC (flow: 25 ml/min; 100% H₂O+0.05% for 20 min; 100% H₂O to 55% H₂O+0.05% TFA/45% ACN; Purosphere® STAR-RP18, 25 mm×250 mm, 10 μm). The pure product containing fractions were combined, the ACN was partly removed, and the residue freeze dried to yield 55 mg of the title compound (Intermediate 16). LC/MS: m/z=333.2 [M+H]⁺; rt: 1.62 min (LC/MS-method A).

### Intermediate 17: Tert-butyl ((1S)-2-((4-(1-(((S)-2-amino-3,3,3-trifluoropropyl)amino)-2-methoxyethyl)pyridin-2-yl)amino)-1-((1r,4S)-4-methylcyclohexyl)-2-oxoethyl)carbamate

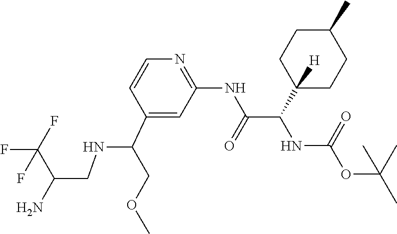

### Step 1: Tert-butyl ((1S)-2-((4-(1-hydroxy-2-methoxyethyl)pyridin-2-yl)amino)-1-((1r,4S)-4-methylcyclohexyl)-2-oxoethyl)carbamate

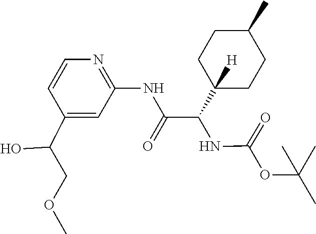

A suspension of HgCl₂ (0.29 g) and Mg (3.05 g) in THE (1.95 ml) was purged with N₂. To the mixture was added a solution of 1,2-dibromoethane (301.41 μl) in THE (1.95 ml) at 25° C. The mixture was allowed to reach 40° C. for 25 min. After the mixture was cooled to 25° C., MOMCl (~6 ml) in THE (18 ml) was added dropwise at 25° C. for 5 min. The mixture was cooled to −25° C. and then further MOMCl (12 ml) in THE (18 ml) was added dropwise at −10 to −15° C. The mixture was stirred at −20° C. for 2 h. A solution of tert-butyl ((S)-2-((4-formylpyridin-2-yl)amino)-1-((1r,4S)-4-methylcyclohexyl)-2-oxoethyl)carbamate (Intermediate 1—step A4, 3.00 g) in THE (13.5 ml) was added to the mixture at −20° C. and stirred at −20° C. for 1 h. The reaction mixture was quenched with saturated NH₄Cl solution (50 ml) and then extracted with EA (30 ml×2). The organic phases were combined, washed with brine (30 ml×2), dried over anhydrous Na₂SO₄, filtered and concentrated in vacuo to give a residue, which was purified by column chromatography (Silica gel, PE/EA=1/1) to yield 2.94 g of the title compound. ¹H NMR (400 MHz, CDCl₃, representative signals shown): δ ppm=8.79-8.68 (m, 1H), 8.30-8.21 (m, 2H), 7.18 (d, J=5.1 Hz, 1H), 5.13 (br d, J=7.3 Hz, 1H), 4.91 (br d, J=6.5 Hz, 2H), 4.18-4.06 (m, 1H), 3.66-3.61 (m, 1H), 3.43 (s, 3H), 1.45 (s, 9H), 0.87 (d, J=6.5 Hz, 3H).

### Step 2: Tert-butyl ((S)-2-((4-(2-methoxyacetyl)pyridin-2-yl)amino)-1-((1r,4S)-4-methylcyclohexyl)-2-oxoethyl)carbamate

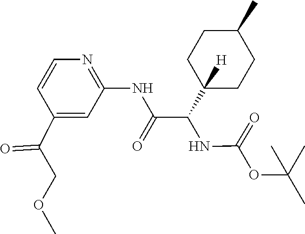

At 0° C., DMP (4.44 g) was slowly added to a solution of tert-butyl ((1S)-2-((4-(1-hydroxy-2-methoxyethyl)-pyridin-2-yl)amino)-1-((1r,4S)-4-methylcyclohexyl)-2-oxoethyl)carbamate (2.94 g) in DCM (30 ml), and the reaction mixture was stirred at 0° C. for 2 h. The reaction mixture was filtered, and the filtrate was washed with saturated sodium hydrogen carbonate solution (20 ml×3) and brine (10 ml). The organic phase was dried over anhydrous sodium sulphate, filtered and evaporated to yield 2.76 g of the title compound. ¹H NMR (400 MHz, CDCl₃, representative signals shown): δ ppm=8.81 (br s, 1H), 8.64 (s, 1H), 8.46 (d, J=5.0 Hz, 1H), 7.51 (d, J=4.9 Hz, 1H), 5.12 (br d, J=7.3 Hz, 1H), 4.72 (s, 2H), 4.16 (br s, 1H), 3.52 (s, 3H), 1.46 (s, 9H), 0.87 (d, J=6.5 Hz, 3H).

### Step 3: Tert-butyl ((1S)-2-((4-(1-(((S)-2-amino-3,3,3-trifluoropropyl)amino)-2-methoxyethyl)pyridin-2-yl)amino)-1-((1r,4S)-4-methylcyclohexyl)-2-oxoethyl)carbamate

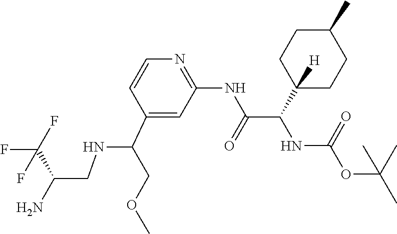

Tert-butyl ((S)-2-((4-(2-methoxyacetyl)pyridin-2-yl)amino)-1-((1r,4S)-4-methylcyclohexyl)-2-oxoethyl)carbamate (2.78 g) and (S)-3,3,3-trifluoropropane-1,2-diamine dihydrochloride [Intermediate 1—Step B6] (1.33 g) are suspended in i-PrOH (28 ml). TEA (2.40 ml) was added and the reaction mixture was stirred at 75° C. for 2 h, then stirred at 25° C. for 12 h. The reaction mixture was concentrated in vacuo and dissolved in MeOH (28 ml), then NaBH₃CN (2.34 g) was added in portions. The resulting suspension was stirred for 5 min, then acetic acid (2.73 ml) was added. The reaction mixture was heated at 40° C. with stirring for 25 min, then cooled and concentrated in vacuo. The residue was diluted with EA (40 ml) and neutralised with saturated aqueous NaHCO₃ solution (50 ml) with vigorous stirring for 0.5 h. The layers were separated and the aqueous layer was extracted with EA (50 ml×2). The combined organic layers were washed with saturated aqueous NaHCO₃ solution (50 ml) and brine (50 ml), dried over anhydrous Na₂SO₄, filtered and concentrated in vacuo to yield 3.61 g of the title compound (Intermediate 17; mixture of diastereomers).

### Intermediate 18: (2S)-2-Amino-N-(4-((R or S)-2-methoxy-1-((S)-2-oxo-4-(trifluoromethyl)imidazolidin-1-yl)ethyl)pyridin-2-yl)-2-((1r,4S)-4-methylcyclohexyl) acetamide hydrochloride salt (DS2)

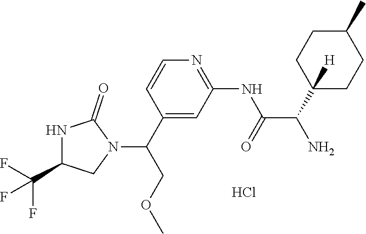

Following the procedure in Intermediate 4, using tert-butyl ((S)-2-((4-((R or S)-2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)carbamate (DS2; Example 66; 420 mg), 383 mg of the title compound was obtained. LC/MS: m/z=458.3 [M+H]⁺; rt: 1.34 min (LC/MS-method A).

### Intermediate 19: (2S)-2-Amino-N-(4-((R or S)-2-methoxy-1-((S)-2-oxo-4-(trifluoromethyl)imidazolidin-1-yl)ethyl)pyridin-2-yl)-2-((1r,4S)-4-methylcyclohexyl)acetamide hydrochloride salt (DS1)

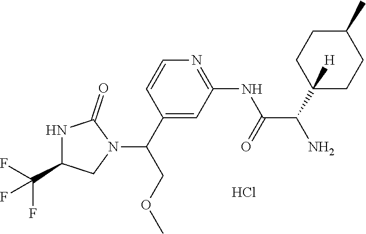

Following the procedure in Intermediate 4, using tert-butyl ((S)-2-((4-((S or R)-2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)carbamate (DS1; Example 65; 420 mg), 389 mg of the title compound was obtained. LC/MS: m/z=458.3 [M+H]⁺; rt: 1.41 min (LC/MS-method A).

### Intermediate 20: (S)-2-(4,4-Difluorocyclohexyl)-2-((((1,5-dimethyl-1H-pyrazol-3-yl)methoxy)carbonyl)amino)acetic acid

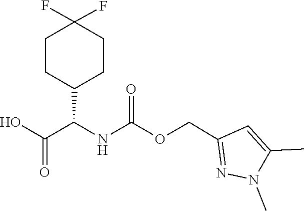 [SMILES: Cc1cc(COC(=O)N[C@H](C(=O)O)C2CCC(F)(F)CC2)nn1C]

### Step 1: Methyl (S)-2-(4,4-difluorocyclohexyl)-2-((((1,5-dimethyl-1H-pyrazol-3-yl)methoxy)carbonyl)-amino)acetate

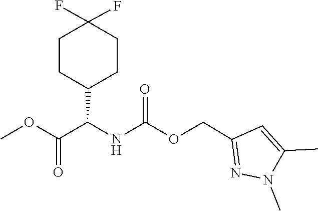

(4-Nitrophenyl) carbonochloridate (50 mg) was dissolved in dry THE (2 ml) under Ar. After cooling to −78° C. methyl (S)-2-amino-2-(4,4-difluorocyclohexyl)acetate (Intermediate 16, step 1; 50 mg, dissolved in dry THE (1 ml)) and DIPEA (63 μl) were added with stirring. After 2.5 h, NaH (25 mg, 60% in mineral oil) was mixed with dry THE (2 ml) under Ar. Then (1,5-dimethylpyrazol-3-yl)methanol (76 mg) dissolved in dry THE (1 ml) was added with stirring. After 25 min, this mixture was dropwise added to the mixture above at −78° C. After 5 min the cooling bath was removed and 1 h later the mixture was poured onto saturated Na₂CO₃ solution. DCM was added and the organic phase was washed with saturated Na₂CO₃ (4×) and finally saturated NaHCO₃ solution. The organic phase was dried over sodium sulphate, filtered and concentrated in vacuo. The residue was purified by preparative RP HPLC (Agilent Prep C18, 30 mm×250 mm, 10 μm; 50 ml/min, from 90% H₂O/10% ACN to 10% H₂O/90% ACN in 12.5 min). The pure product containing fractions were combined, the ACN was partly removed and the residue freeze dried to yield 25 mg of the title compound. LC/MS: m/z=360.2 [M+H]⁺; rt: 1.82 min (LC/MS-method A)

### Step 2: (S)-2-(4,4-Difluorocyclohexyl)-2-((((1,5-dimethyl-1H-pyrazol-3-yl)methoxy)carbonyl)amino)acetic acid

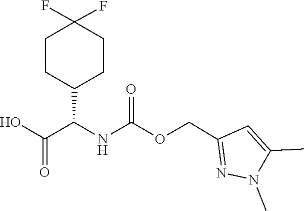 [SMILES: Cc1cc(COC(=O)N[C@H](C(=O)O)C2CCC(F)(F)CC2)nn1C]

Methyl (S)-2-(4,4-difluorocyclohexyl)-2-((((1,5-dimethyl-1H-pyrazol-3-yl)meth-oxy)carbonyl)amino)-acetate (12 mg) was dissolved in THE/water (0.8/0.2 ml) and LiOH (2 mg) was added with stirring. After 1 h, aqueous HCl was added (1 M) to adjust the pH to 2-3. Then the aqueous phase was extracted with EA (2×). The combined organic phases were dried (Chem Elut cartridge) and concentrated in vacuo to yield 10 mg of the title compound (Intermediate 20). LC/MS: m/z=346.2 [M+H]⁺; rt: 1.56 min (LC/MS-method A).

### Intermediate 21: (S)-2-Amino-N-(5-fluoro-4-(((S)-2-oxo-4-(trifluoromethyl)-imidazolidin-1-yl)methyl)pyridin-2-yl)-2-((1r,4S)-4-methylcyclohexyl)acetamide hydrochloride salt

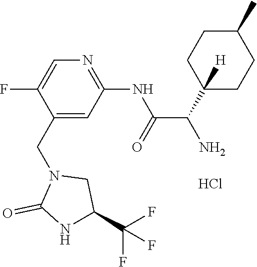

Following the procedure in Intermediate 4, using tert-butyl ((S)-2-((5-fluoro-4-(((S)-2-oxo-4-(trifluoromethyl)imidazolidin-1-yl)methyl)pyridin-2-yl)amino)-1-((1r,4S)-4-methylcyclohexyl)-2-oxoethyl)carbamate (Example 50; 342 mg), 312 mg of the title compound was obtained. LC/MS: m/z=432.2 [M+H]⁺; rt: 1.38 min (LC/MS-method A).

### Intermediate 22: (S)-2-(4,4-Difluorocyclohexyl)-2-((((4-methyl-1,2,5-oxadiazol-3-yl)methoxy)carbonyl)amino)acetic acid

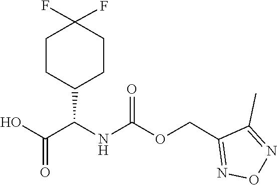 [SMILES: Cc1nonc1COC(=O)N[C@H](C(=O)O)C1CCC(F)(F)CC1]

Following the sequence described for Intermediate 20, using (4-methyl-1,2,5-oxadiazol-3-yl)methanol (71 mg), 34 mg of the title compound was obtained. LC/MS: m/z=334.1 [M+H]⁺; rt: 1.68 min (LC/MS-method A).

### Intermediate 23: Tert-butyl ((S)-2-((4-(bromomethyl)pyridin-2-yl)amino)-1-((1r,4S)-4-methylcyclohexyl)-2-oxoethyl)carbamate

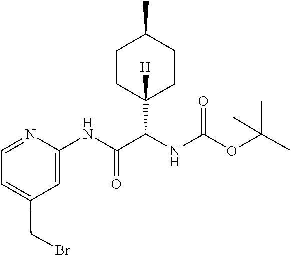

Following the procedure of Intermediate 11, using tert-butyl ((S)-2-((4-(hydroxymethyl)pyridin-2-yl)amino)-1-((1r,4S)-4-methylcyclohexyl)-2-oxoethyl)carbamate (Intermediate 1—Step A3; 41 mg), 12 mg of the title compound was obtained. LC/MS: m/z=440.2 [M+H]⁺; rt: 2.71 min (LC/MS-method A).

### Intermediate 24: (S)-1-((5-Aminopyridin-3-yl)methyl)-4-(trifluoromethyl)-imidazolidin-2-one

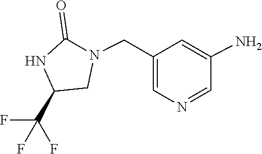 [SMILES: Nc1cncc(CN2C[C@@H](C(F)(F)F)NC2=O)c1]

Following the sequence to Intermediate 15, and using tert-butyl N-(5-formyl-3-pyridyl)carbamate as starting material (500 mg), 283 mg of the title compound was obtained. LC/MS: m/z=261.1 [M+H]⁺; rt: 0.37 min (LC/MS-method A).

### Intermediate 25: Rac-2-amino-3-(tert-butoxy)-N-(4-(((S)-2-oxo-4-(trifluoromethyl)-imidazolidin-1-yl)methyl)pyridin-2-yl)propanamide

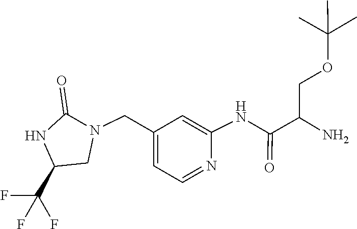 [SMILES: CC(C)(C)OCC(N)C(=O)Nc1cc(CN2C[C@@H](C(F)(F)F)NC2=O)ccn1]

Benzyl ((rac)-3-(tert-butoxy)-1-oxo-1-((4-(((S)-2-oxo-4-(trifluoromethyl)imidazolidin-1-yl)methyl)pyridin-2-yl)amino)propan-2-yl)carbamate (Example 81, 30 mg) was dissolved in MeOH (2 ml) under Ar. Pd/C (10%, 3 mg) was added and the flask was purged with H₂ from a balloon. After stirring for 2 h under a H₂ atmosphere the mixture was filtered, the filter washed with MeOH and the filtrate concentrated in vacuo to yield 23 mg of the title compound. LC/MS: m/z=404.2 [M+H]⁺; rt: 1.12 min (LC/MS-method A).

### Intermediate 26: (S)-2-Amino-2-((1r,4S)-4-methylcyclohexyl)-N-(5-(((S)-2-oxo-4-(trifluoromethyl)imidazolidin-1-yl)methyl)pyridin-3-yl)acetamide and (R)-2-amino-2-((1r,4R)-4-methylcyclohexyl)-N-(5-(((S)-2-oxo-4-(trifluoromethyl)imidazolidin-1-yl)methyl)pyridin-3-yl)acetamide

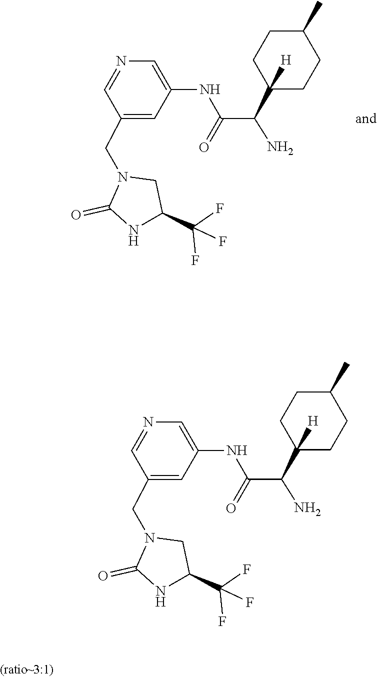

Following the procedure of Intermediate 2, and using tert-butyl ((S)-1-((1r,4S)-4-methylcyclohexyl)-2-oxo-2-((5-(((S)-2-oxo-4-(trifluoromethyl)imidazolidin-1-yl)methyl)pyridin-3-yl)amino)ethyl)carbamate and tert-butyl ((R)-1-((1r,4R)-4-methylcyclohexyl)-2-oxo-2-((5-(((S)-2-oxo-4-(trifluoromethyl)imidazolidin-1-yl)methyl)pyridin-3-yl)amino)ethyl)carbamate (Example 80; 99 mg), 87 mg of the crude title compounds was obtained (ratio ~3:1). LC/MS: m/z=414.2 [M+H]⁺; rt: 1.18 min (LC/MS-method A).

### Intermediate 27: (2S,3R)-2-Amino-3-(tert-butoxy)-N-(4-(((S)-2-oxo-4-(trifluoromethyl)imidazolidin-1-yl)methyl)pyridin-2-yl)butanamide

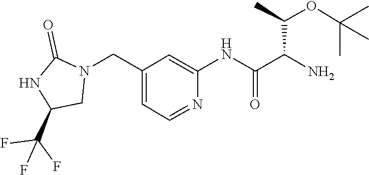 [SMILES: C[C@@H](OC(C)(C)C)[C@H](N)C(=O)Nc1cc(CN2C[C@@H](C(F)(F)F)NC2=O)ccn1]

Following the procedure of Intermediate 25, and using benzyl ((2S,3R)-3-(tert-butoxy)-1-oxo-1-((4-(((S)-2-oxo-4-(trifluoromethyl)imidazolidin-1-yl)methyl)pyridin-2-yl)amino)butan-2-yl)carbamate (Example 101; 30 mg) 23 mg of the crude title compound was obtained. LC/MS: m/z=418.1 [M+H]⁺; rt: 0.62 min (LC/MS-method B).

### Intermediate 28: Tert-butyl ((S)-1-((4-((((S)-2-amino-3,3,3-trifluoropropyl)-amino)methyl)pyridin-2-yl)amino)-3,3-dicyclopropyl-1-oxopropan-2-yl)carbamate

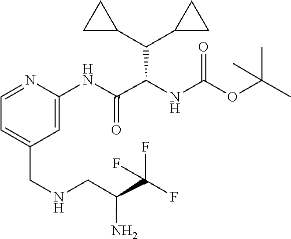 [SMILES: CC(C)(C)OC(=O)N[C@H](C(=O)Nc1cc(CNC[C@H](N)C(F)(F)F)ccn1)C(C1CC1)C1CC1]

Tert-butyl ((S)-1-((4-((((S)-2-amino-3,3,3-trifluoropropyl)amino)methyl)pyridin-2-yl)amino)-3,3-dicyclopropyl-1-oxopropan-2-yl)carbamate TFA salt (Intermediate 5) was dissolved in a mixture of ACN/water (0.05% TFA). The TFA was neutralised by saturated NaHCO₃ solution. After extracting the product with DCM (3×), the combined organic phases were dried over sodium sulphate, filtered and concentrated in vacuo. The residue was dissolved in ACN/H₂O and freeze dried to yield 183 mg of the title compound. LC/MS: m/z=486.4 [M+H]⁺; rt: 1.59 min (LC/MS-method A).

### Intermediate 29: (2S,3S)-2-Amino-3-(tert-butoxy)-N-(4-(((S)-2-oxo-4-(trifluoromethyl)imidazolidin-1-yl)methyl)pyridin-2-yl)butanamide

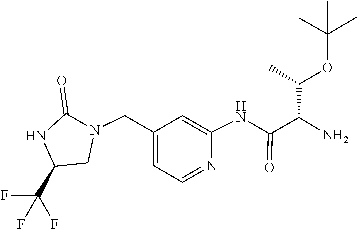 [SMILES: C[C@H](OC(C)(C)C)[C@H](N)C(=O)Nc1cc(CN2C[C@@H](C(F)(F)F)NC2=O)ccn1]

### Step 1: Benzyl ((2S,3S)-3-(tert-butoxy)-1-oxo-1-((4-(((S)-2-oxo-4-(trifluoromethyl)-imidazolidin-1-yl)methyl)pyridin-2-yl)amino)butan-2-yl)carbamate

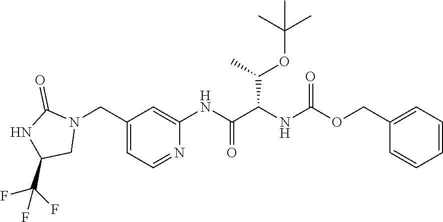 [SMILES: C[C@H](OC(C)(C)C)[C@H](NC(=O)OCc1ccccc1)C(=O)Nc1cc(CN2C[C@@H](C(F)(F)F)NC2=O)ccn1]

To N-((benzyloxy)carbonyl)-O-(tert-butyl)-L-allothreonine (400 mg) was added DCM (6 ml), DMAP (221 mg) and EDCl (347 mg) with stirring. After 5 min (4S)-1-[(2-amino-4-pyridyl)methyl]-4-(trifluoromethyl)imidazolidin-2-one (Intermediate 15; 235 mg) was added and stirring was continued for 1 h. Then the mixture was poured onto saturated NaHCO₃-solution. The aqueous phase was extracted with DCM (2×). The combined organic phases were dried with magnesium sulphate, filtered and concentrated in vacuo. The residue was purified by silica gel chromatography (Silica gel 24 g, DCM/EtOH: 100:0 for 5 min, 100:0 to 95:5 in 30 min, 95:5 for 10 min). Product containing fractions were combined and concentrated in vacuo. The residue was further purified by preparative RP HPLC (flow: 15 ml/min; gradient: 95% H₂O+0.05% TFA/5% ACN in 45 min to 5% H₂O+0.05% TFA/95% ACN; column: Xbridge, BEH130, Prep. C18, OBD; 19 mm×250 mm, 10 μm). The product containing fractions were combined, the ACN was removed, saturated NaHCO₃ solution was added (pH~7) and the aqueous mixture was extracted with DCM (2×). The combined organic phases were dried over sodium sulphate, filtered and concentrated in vacuo. The residue was dissolved in ACN/water and freeze dried to yield 56 mg of the title compound. LC/MS: m/z=552.3 [M+H]⁺; rt: 2.18 min (LC/MS-method A).

### Step 2: (2S,3S)-2-Amino-3-(tert-butoxy)-N-(4-(((S)-2-oxo-4-(trifluoromethyl)imidazolidin-1-yl)methyl)pyridin-2-yl)butanamide

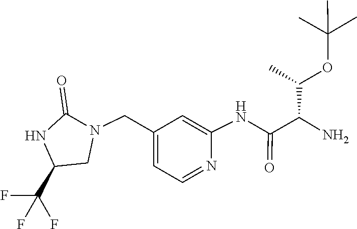 [SMILES: C[C@H](OC(C)(C)C)[C@H](N)C(=O)Nc1cc(CN2C[C@@H](C(F)(F)F)NC2=O)ccn1]

Following the procedure of Intermediate 25, and using benzyl ((2S,3S)-3-(tert-butoxy)-1-oxo-1-((4-(((S)-2-oxo-4-(trifluoromethyl)imidazolidin-1-yl)methyl)pyridin-2-yl)amino)butan-2-yl)carbamate (60 mg), 43 mg of the crude title compound (Intermediate 29) was obtained and used without further purification. LC/MS: m/z=418.2 [M+H]⁺; rt: 1.16 min (LC/MS-method A).

### Intermediate 30: Tert-butyl (S)-(1,1-dicyclopropyl-3-oxo-3-((4-(2-oxo-2-((3,3,3-trifluoropropyl)amino)ethyl)pyridin-2-yl)amino)propan-2-yl)carbamate

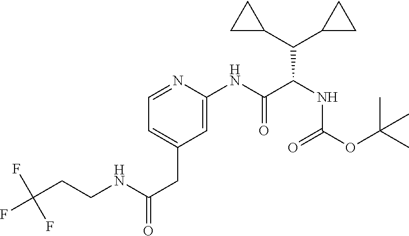 [SMILES: CC(C)(C)OC(=O)N[C@H](C(=O)Nc1cc(CC(=O)NCCC(F)(F)F)ccn1)C(C1CC1)C1CC1]

### Step 1: Tert-butyl (4-(2-oxo-2-((3,3,3-trifluoropropyl)amino)ethyl)pyridin-2-yl)carbamate

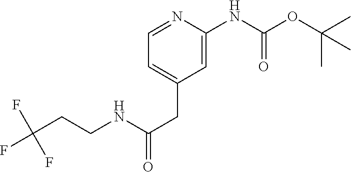 [SMILES: CC(C)(C)OC(=O)Nc1cc(CC(=O)NCCC(F)(F)F)ccn1]

2-[2-(tert-butoxycarbonylamino)-4-pyridyl]acetic acid (505 mg) was dissolved in 8 ml dry DMF, and 1.15 g EDC and ethyl cyanohydroxyiminoacetate (Oxyma; 853 mg) were added together with 1.01 g NaHCO₃ and stirred for 30 min at ambient temperature. Then 300 mg 3,3,3-trifluoropropan-1-amine HCl was dissolved in 2 ml dry DMF and added dropwise and the mixture stirred for 16 h at ambient temperature and the solvent was removed by rotary evaporator. The crude reaction mixture was stirred with 50 ml water and the pH was adjusted to pH 11 with 1 N NaOH and the product filtered off, washed with water and dried to yield 660 mg. LC/MS: m/z=348.1 [M+H]⁺; rt: 0.92 min (LC/MS-method B).

### Step 2: Tert-butyl (S)-(1,1-dicyclopropyl-3-oxo-3-((4-(2-oxo-2-((3,3,3-trifluoropropyl)amino)ethyl)pyridin-2-yl)amino)propan-2-yl)carbamate

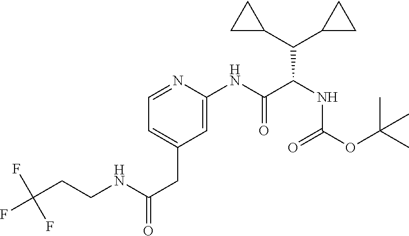 [SMILES: CC(C)(C)OC(=O)N[C@H](C(=O)Nc1cc(CC(=O)NCCC(F)(F)F)ccn1)C(C1CC1)C1CC1]

tert-butyl (4-(2-oxo-2-((3,3,3-trifluoropropyl)amino)ethyl)pyridin-2-yl)carbamate (225 mg) was dissolved in 10 ml dry dioxane and 1.62 ml 4 M HCl in dioxane was added and the mixture stirred for 20 h at ambient temperature, before addition of additional 0.81 ml 4 M HCl in dioxane. The mixture was then stirred for an additional 8 h at ambient temperature. The liquid was decanted and the residue stirred in 10 ml acetonitrile before the solid was filtered off and the liquid phase was evaporated to dryness by rotary evaporation. 120 mg of the crude product was dissolved in 1 ml dry DMF and added dropwise to a solution of 110 mg (2S)-2-(tert-butoxycarbonylamino)-3,3-dicyclopropyl-propanoic acid in 4 ml dry DMF with 233 mg HATU and 0.36 ml DIPEA and the mixture stirred by ambient temperature for 16 h. The reaction mixture was evaporated to dryness and redissolved in 20 ml EA and the organic phase washed with 10 ml water, 10 ml saturated NaHCO₃ (aq) and 10 ml brine and dried over MgSO₄ before being evaporated to dryness. The crude product (Intermediate 30) was used directly without further purification. LC/MS: m/z=499.3 [M+H]⁺; rt: 1.33 min (LC/MS-method B).

### Intermediate 31: Tert-butyl (S)-(1,1-dicyclopropyl-3-oxo-3-((4-((4,4,4-trifluoro-butanamido)methyl)pyridin-2-yl)amino)propan-2-yl)carbamate

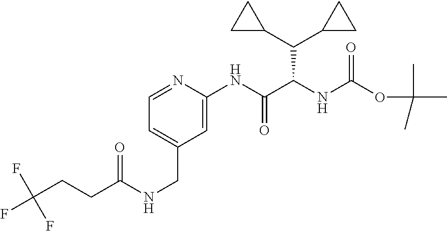 [SMILES: CC(C)(C)OC(=O)N[C@H](C(=O)Nc1cc(CNC(=O)CCC(F)(F)F)ccn1)C(C1CC1)C1CC1]

### Step 1: Tert-butyl (4-((4,4,4-trifluorobutanamido)methyl)pyridin-2-yl)carbamate

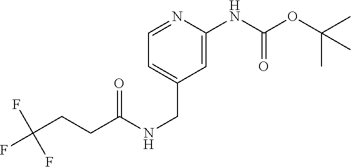 [SMILES: CC(C)(C)OC(=O)Nc1cc(CNC(=O)CCC(F)(F)F)ccn1]

4,4,4-trifluorobutanoic acid (286 mg) was dissolved in 5 ml dry DMF and 1.15 g HATU and 1.05 ml DIPEA were added and the mixture stirred for 30 min at ambient temperature. Then tert-butyl N-[4-(aminomethyl)-2-pyridyl]carbamate (450 mg) in 5 ml dry DMF was added and the mixture stirred for 16 h at ambient temperature. The reaction mixture was evaporated to dryness and dissolved in 50 ml EA and washed with 30 ml water, 30 ml saturated NaHCO₃ (aq), and 30 ml brine and dried over MgSO4 before being evaporated to dryness. LC/MS: m/z=348.1 [M+H]⁺; rt: 1.54 min (LC/MS-method A).

### Step 2: Tert-butyl (S)-(1,1-dicyclopropyl-3-oxo-3-((4-((4,4,4-trifluorobutan-amido)methyl)pyridin-2-yl)amino)propan-2-yl)carbamate

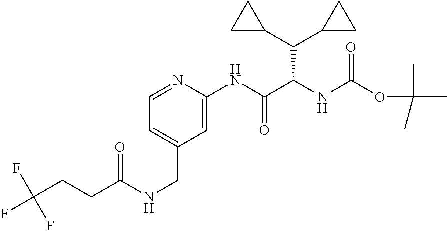 [SMILES: CC(C)(C)OC(=O)N[C@H](C(=O)Nc1cc(CNC(=O)CCC(F)(F)F)ccn1)C(C1CC1)C1CC1]

Tert-butyl (4-((4,4,4-trifluorobutanamido)methyl)pyridin-2-yl)carbamate (630 mg) was dissolved in 120 ml dry DMF together with 1.86 g TsOH and the mixture stirred for 16 h at ambient temperature before being evaporated to dryness. The residue was dissolved in 18.2 ml 0.5 M NaOH (aq) and extracted 6 times with 20 ml DCM and the combined organic phases were dried over MgSO₄, filtered evaporated to dryness. 115 mg of the crude product was dissolved in 1 ml dry DMF and added dropwise to a solution of (2S)-2-(tert-butoxycarbonylamino)-3,3-dicyclopropyl-propanoic acid (125 mg) in 4 ml dry DMF with 265 mg HATU and 0.24 ml DIPEA and the mixture stirred by ambient temperature for 16 h. The reaction mixture was evaporated to dryness and redissolved in 20 ml EA and the organic phase washed with 10 ml water, 10 ml saturated NaHCO₃ (aq), and 10 ml brine and dried over MgSO4 before being evaporated to dryness. The crude product (Intermediate 31) was used directly without further purification. LC/MS: m/z=499.3 [M+H]⁺; rt: 1.33 min (LC/MS-method B).

### Intermediate 32: (S)-2-Amino-3,3-dicyclopropyl-N-(4-(((R)-4-methyl-2-oxoimidazolidin-1-yl)methyl)pyridin-2-yl)propenamide HCl salt

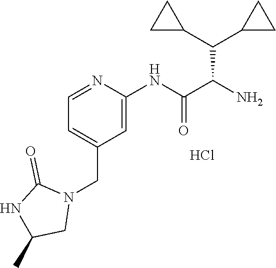 [SMILES: C[C@@H]1CN(Cc2ccnc(NC(=O)[C@@H](N)C(C3CC3)C3CC3)c2)C(=O)N1 ; Cl]

100 mg of the title compound was prepared analogously to Intermediate 4. LC/MS: m/z=358.2 [M+H]⁺; rt: 0.79 min (LC/MS-method B).

### Intermediate 33: 2-Amino-2-(4,4-difluorocyclohexyl)-N-(4-((1-methyl-6-oxo-1,6-dihydropyridin-2-yl)methyl)pyridin-2-yl)acetamide HCl salt

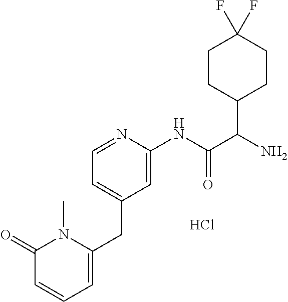 [SMILES: Cn1c(Cc2ccnc(NC(=O)C(N)C3CCC(F)(F)CC3)c2)cccc1=O ; Cl]

### Step 1: 1-Methyl-6-(4,4,5,5-tetramethyl-1,3,2-dioxaborolan-2-yl)pyridin-2(1H)-one

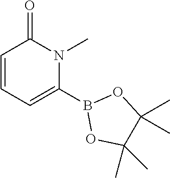 [SMILES: Cn1c(B2OC(C)(C)C(C)(C)O2)cccc1=O]

To a solution of 6-bromo-1-methylpyridin-2(1H)-one (8 g, 42.55 mmol, 1 eq) in dioxane (200 ml) were added BPD (32.41 g, 127.64 mmol, 3 eq), KOAc (12.53 g, 127.68 mmol, 3.00 eq) and Pd(dppf)Cl₂ (3.74 g, 5.11 mmol, 0.12 eq), at 25° C. under nitrogen. The reaction mixture was stirred at 110° C. for 3 h under nitrogen atmosphere. The reaction mixture was filtered, and the filter cake was washed with warm toluene (40° C., 5 times 100 ml). The combined organic phases were concentrated under reduced pressure to provide 1-methyl-6-(4,4,5,5-tetramethyl-1,3,2-dioxaborolan-2-yl)pyridin-2(1H)-one (15 g, crude) as a black oil that was used directly in the next step.

### Step 2: Tert-butyl (4-((1-methyl-6-oxo-1,6-dihydropyridin-2-yl)methyl)pyridin-2-yl)carbamate

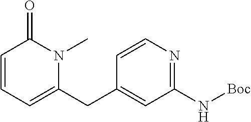 [SMILES: Cn1c(Cc2ccnc(NC(=O)OC(C)(C)C)c2)cccc1=O]

To a mixture of 1-methyl-6-(4,4,5,5-tetramethyl-1,3,2-dioxaborolan-2-yl)pyridin-2(1H)-one (14 g, 59.55 mmol, 1 eq) and tert-butyl (4-(bromomethyl)pyridin-2-yl)carbamate (4 g, 13.93 mmol, 0.23 eq) in dioxane (182 ml) was added K₂CO₃ (2 M, 74.44 mL, 2.5 eq) and Pd(dppf)Cl2 (4.36 g, 5.96 mmol, 0.1 eq) in one portion at 25° C. under N₂. The mixture was stirred at 110° C. for 2 h. The reaction mixture was poured into H₂O (100 ml), and extracted with EA (3×150 ml). The combined organic phase was washed with brine (50 ml), dried over anhydrous Na₂SO₄, filtered and evaporated to dryness. The residue was purified by FC on silica gel using PE:DCM to give 2.2 g of the title product as a yellow solid. ¹H NMR (400 MHz, CDCl₃) δ=8.16 (d, J=5.2 Hz, 1H), 7.85 (s, 1H), 7.62 (br s, 1H), 7.27-7.24 (m, 1H), 6.65 (d, J=5.2 Hz, 1H), 6.52 (d, J=8.4 Hz, 1H), 6.00 (d, J=6.4 Hz, 1H), 3.93 (s, 2H), 3.38 (s, 3H), 1.50 (s, 9H).

### Step 3: Tert-butyl (1-(4,4-difluorocyclohexyl)-2-((4-((1-methyl-6-oxo-1,6-dihydropyridin-2-yl)methyl)pyridin-2-yl)amino)-2-oxoethyl)carbamate

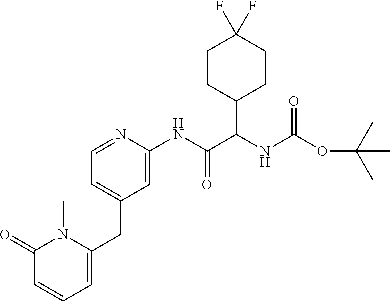 [SMILES: Cn1c(Cc2ccnc(NC(=O)C(NC(=O)OC(C)(C)C)C3CCC(F)(F)CC3)c2)cccc1=O]

Tert-butyl (4-((1-methyl-6-oxo-1,6-dihydropyridin-2-yl)methyl)pyridin-2-yl)-carbamate (1.0 g) was dissolved in 10 ml dry dioxane, and 7.9 ml 4 N HCl in dioxane was added dropwise, and the mixture stirred overnight at ambient temperature before the precipitate was filtered off. The solid was taken up in NaHCO₃ (aq, sat.) and extracted 3 times with 10 ml EA. The combined organic phases were dried over Na₂SO₄, filtered and evaporated to dryness to afford 6-[(2-amino-4-pyridyl)methyl]-1-methyl-pyridin-2-one (256 mg). This residue was dissolved in 2 ml DMF and added to a mixture of 2-(tert-butoxycarbonylamino)-2-(4,4-difluorocyclohexyl)acetic acid (349 mg), 498 mg HATU, and 308 mg DIPEA in 10 ml DMF. The reaction mixture was stirred for 16 h and then evaporated to dryness, redissolved in 30 ml EA and washed with 10 ml water, 10 ml NaHCO₃ (aq, sat), and 10 ml brine. The organic phase was dried over anhydrous Na₂SO₄, filtered and evaporated to dryness. The residue was purified by FC on silica gel using MeOH:DCM to give the 236 mg of the title product. LC/MS: m/z=491.2 [M+H]⁺; rt: 1.92 min (LC/MS-method A).

### Step 4: 2-Amino-2-(4,4-difluorocyclohexyl)-N-(4-((1-methyl-6-oxo-1,6-dihydropyridin-2-yl)methyl)pyridin-2-yl)acetamide

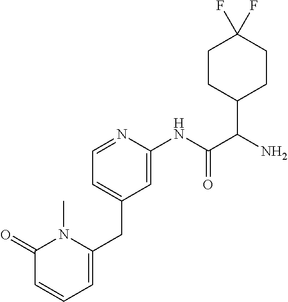 [SMILES: Cn1c(Cc2ccnc(NC(=O)C(N)C3CCC(F)(F)CC3)c2)cccc1=O]

Tert-butyl (1-(4,4-difluorocyclohexyl)-2-((4-((1-methyl-6-oxo-1,6-dihydropyridin-2-yl)methyl)pyridin-2-yl)amino)-2-oxoethyl)carbamate (230 mg) was dissolved in 5 ml dry dioxane, and 1.17 ml 4 N HCl in dioxane was added dropwise and the mixture stirred overnight at ambient temperature. The reaction mixture was dissolved with 20 ml DCM and washed 3 times with Na₂CO₃ (aq, sat.) before the organic phase was dried over anhydrous Na₂SO₄, filtered and evaporated to dryness to afford 138 mg of the title product (Intermediate 33) that was used without further purification.

### Intermediate 34 diastereomer 1: N-((2-((S)-2-amino-3,3-dicyclopropylpropanamido)pyridin-4-yl)(cyclopropyl)methyl)-4,4,4-trifluorobutanamide (DS1)

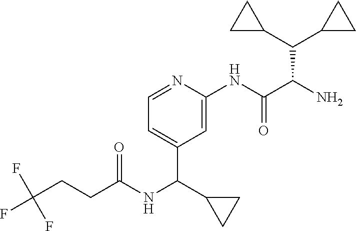 [SMILES: N[C@H](C(=O)Nc1cc(C(NC(=O)CCC(F)(F)F)C2CC2)ccn1)C(C1CC1)C1CC1]

### Step 1: Tert-butyl (R,E)-(4-(((tert-butylsulfinyl)imino)methyl)pyridin-2-yl)carbamate

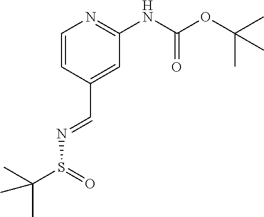 [SMILES: CC(C)(C)OC(=O)Nc1cc(/C=N/[S@](=O)C(C)(C)C)ccn1]

255 mg (R)-2-methylpropane-2-sulfinamide was dissolved in 10 ml dry THE and 450 mg tert-butyl N-(4-formyl-2-pyridyl)carbamate, 1.02 g CuSO₄, and 50 mg pyridinium p-toluenesulfonate were added, and the reaction mixture stirred at 55° C. for 16 h. The reaction mixture was cooled, diluted with 20 ml DCM, and stirred over MgSO₄ before being filtered and evaporated to dryness before the crude product (570 mg) was used directly in the next step. LC/MS: m/z=326.2 [M+H]⁺; rt: 2.19 min (LC/MS-method A)

### Step 2: Tert-butyl (4-((((R)-tert-butylsulfinyl)amino)(cyclopropyl)methyl)pyridin-2-yl)carbamate

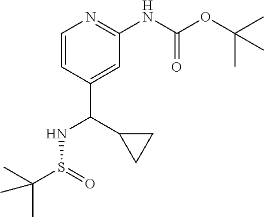 [SMILES: CC(C)(C)OC(=O)Nc1cc(C(N[S@](=O)C(C)(C)C)C2CC2)ccn1]

570 mg tert-butyl (R,E)-(4-(((tert-butylsulfinyl)imino)methyl)pyridin-2-yl)carbamate was dissolved in 20 ml dry DCM under an argon atmosphere and the mixture cooled to 0° C. and 8.0 ml bromo(cyclopropyl)magnesium (0.5 M) was added dropwise keeping the temperature below 5° C. The reaction mixture was stirred for 4 h at ambient temperature before another 8.0 ml bromo(cyclopropyl)magnesium (0.5M) was added, and the mixture stirred for 16 h before the mixture was quenched with saturated ammonium chloride solution (aq) and further diluted with 20 ml water. The phases were separated and the aqueous phase extracted twice with DCM before the combined organic phases were dried over MgSO₄, filtered and evaporated to dryness before the crude product (680 mg) was used directly in the next step. LC/MS: m/z=368.2 [M+H]⁺; rt: 1.85 min (LC/MS-method A).

### Step 3: tert-butyl (R)-(4-(amino(cyclopropyl)methyl)pyridin-2-yl)carbamate

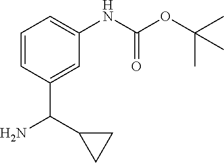 [SMILES: CC(C)(C)OC(=O)Nc1cccc(C(N)C2CC2)c1]

680 mg tert-butyl (4-((((R)-tert-butylsulfinyl)amino)(cyclopropyl)methyl)pyridin-2-yl)carbamate was dissolved in 20 ml THE and 4 ml water before addition of 223 mg iodine and the mixture heated to 60° C. for 2 h. After cooling, the mixture was diluted with 50 ml EA and washed twice with 25 ml of 5% NaHCO₃ (aq) and 10% Na₂S2O₃ (aq) as well as once with 20 ml brine. The organic phase was dried over MgSO₄, filtered and evaporated to dryness before the crude product (380 mg) was used directly in the next step. LC/MS: m/z=264.2 [M+H]⁺; rt: 1.07 min (LC/MS-method A).

### Step 4: Tert-butyl (4-(cyclopropyl(4,4,4-trifluorobutanamido)methyl)pyridin-2-yl)carbamate

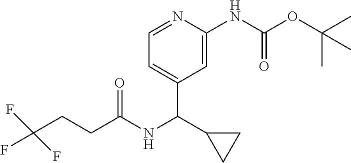 [SMILES: CC(C)(C)OC(=O)Nc1cc(C(NC(=O)CCC(F)(F)F)C2CC2)ccn1]

205 mg 4,4,4-trifluorobutanoic acid was diluted in 10 ml dry DMF and 820 mg HATU, 0.75 ml DIPEA and 380 mg tert-butyl (R)-(4-(amino(cyclopropyl)methyl)pyridin-2-yl)carbamate dissolved in 2 ml dry DMF were added, and the mixture stirred for 16 h at ambient temperature. The reaction mixture was evaporated to dryness and the residue dissolved in 50 ml EA and washed with 30 ml of water, NaHCO₃ (aq, sat.) and brine before being dried over MgSO₄, filtered, and evaporated to dryness before the crude product (540 mg) was used directly in the next step. LC/MS: m/z=388.1 [M+H]⁺; rt: 1.87 min (LC/MS-method A).

### Step 5: N-((2-aminopyridin-4-yl)(cyclopropyl)methyl)-4,4,4-trifluorobutanamide

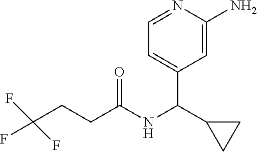 [SMILES: Nc1cc(C(NC(=O)CCC(F)(F)F)C2CC2)ccn1]

540 mg tert-butyl (4-(cyclopropyl(4,4,4-trifluorobutanamido)methyl)pyridin-2-yl)carbamate was dissolved in 15 ml dry dioxane and 3.5 ml 4 N HCl in dioxane was added and the mixture stirred for 16 h at ambient temperature. Then another 3.5 ml 4 N HCl in dioxane was added, and the mixture stirred for another 6 h at ambient temperature before addition of additional 1.8 ml 4N HCl in dioxane. The mixture was stirred for another 6 h at ambient temperature before addition of 30 ml MTBE to precipitate the product. The solvent was decanted and 20 ml DCM added. The mixture stirred for 30 min before the solvent was decanted and the solid dried under vacuum. The crude product was dissolved in 50 ml EA and washed with NaHCO₃ (aq. sat), and dried over MgSO₄, filtered and evaporated to dryness before the crude product (250 mg) was used directly in the next step. LC/MS: m/z=288.1 [M+H]⁺; rt: 0.75 min (LC/MS-method B).

### Step 6: tert-butyl ((2S)-1,1-dicyclopropyl-3-((4-(cyclopropyl(4,4,4-trifluorobutanamido)-methyl)pyridin-2-yl)amino)-3-oxopropan-2-yl)carbamate

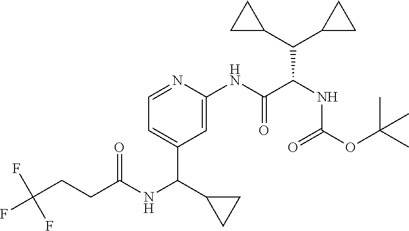 [SMILES: CC(C)(C)OC(=O)N[C@H](C(=O)Nc1cc(C(NC(=O)CCC(F)(F)F)C2CC2)ccn1)C(C1CC1)C1CC1]

95 mg (2S)-2-(tert-butoxycarbonylamino)-3,3-dicyclopropyl-propanoic acid was dissolved in 6 ml dry DCM and 255 mg TBTU and 0.31 ml DIPEA were added, and the mixture stirred for 30 min at ambient temperature. Then 100 mg N-((2-aminopyridin-4-yl)(cyclopropyl)methyl)-4,4,4-trifluorobutanamide dissolved in 2 ml dry DMF was added, and the mixture stirred for 16 h at ambient temperature before being diluted with 20 ml DCM. The mixture was washed with 20 ml water, 20 ml NaHCO₃ (aq. sat) and 20 ml brine before being dried over MgSO₄, filtered stirred twice in 5 ml cyclohexane, decanted and the solid dried under vacuum before the crude product (200 mg) was used directly in the next step. LC/MS: m/z=539.3 [M+H]⁺; rt: 1.49 min (LC/MS-method B).

### Step 7: N-((2-((S)-2-amino-3,3-dicyclopropylpropanamido)-pyridin-4-yl)(cyclopropyl)methyl)-4,4,4-trifluorobutanamide (DS1)

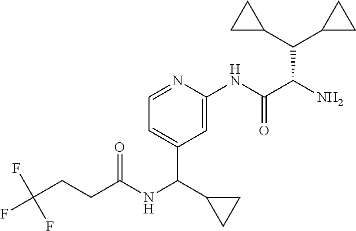 [SMILES: N[C@H](C(=O)Nc1cc(C(NC(=O)CCC(F)(F)F)C2CC2)ccn1)C(C1CC1)C1CC1]

200 mg tert-butyl ((2S)-1,1-dicyclopropyl-3-((4-(cyclopropyl(4,4,4-trifluorobutan-amido)methyl)pyridin-2-yl)amino)-3-oxopropan-2-yl)carbamate was dissolved in 30 ml dry DCM, and 360 mg TsOH was added, and the mixture stirred for 16 h at ambient temperature before evaporation to dryness. The residue was dissolved in 3.6 ml 0.5 M aqueous NaOH solution and extracted 6 times with 5 ml DCM. The combined organic phases were dried over MgSO₄, filtered and evaporated before the crude product (Intermediate 34 diastereomer 1) was used directly without further purification. LC/MS: m/z=439.2 [M+H]⁺; rt: 1.00 min (LC/MS-method B).

### Intermediate 34 diastereomer 2: N-((2-((S)-2-amino-3,3-dicyclopropylpropanamido)-pyridin-4-yl)(cyclopropyl)methyl)-4,4,4-trifluorobutanamide

Diastereomer 2 was prepared analogously to diastereomer 1 using (S)-2-methylpropane-2-sulfinamide in Step 1. LC/MS: m/z=439.2 [M+H]⁺; RT: 1.24 min (LC/MS-method B).

### Intermediate 35 diastereomer 1: N-((2-(2-amino-2-(4,4-difluorocyclohexyl)-acetamido)pyridin-4-yl)(cyclopropyl)methyl)-4,4,4-trifluorobutanamide

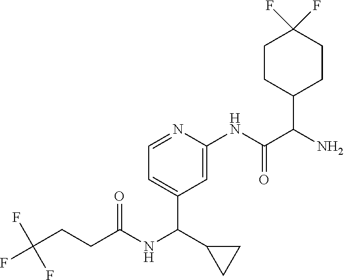 [SMILES: NC(C(=O)Nc1cc(C(NC(=O)CCC(F)(F)F)C2CC2)ccn1)C1CCC(F)(F)CC1]

The title compound was prepared following the procedure for the preparation of Intermediate 34 diastereomer 1, using 2-(tert-butoxycarbonylamino)-2-(4,4-difluorocyclohexyl)acetic acid and N-((2-aminopyridin-4-yl)(cyclopropyl)methyl)-4,4,4-trifluorobutanamide (Intermediate 34 DS1—Step 5), to afford 300 mg product that was used directly in the next step. LC/MS: m/z=463.2 [M+H]⁺; RT: 0.98 min (LC/MS-method B).

### Intermediate 36: 4-((1,4-dimethyl-1H-pyrazol-5-yl)methyl)pyridin-2-amine

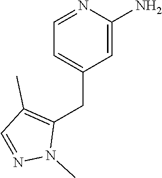

### Step 1: tert-butyl (4-((1,4-dimethyl-1H-pyrazol-5-yl)methyl)pyridin-2-yl)carbamate

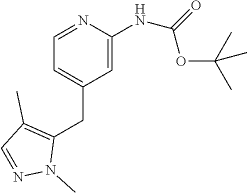 [SMILES: Cc1cnn(C)c1Cc1ccnc(NC(=O)OC(C)(C)C)c1]

1,4-dimethyl-5-(4,4,5,5-tetramethyl-1,3,2-dioxaborolan-2-yl)pyrazole (232 mg) was dissolved in 22.5 ml dioxane, and 2.5 ml water and 567 Cs₂CO₃ was added together with 51 mg Pd(dppf)Cl₂ and 200 mg tert-butyl N-[4-(bromomethyl)-2-pyridyl]carbamate and the mixture heated to 110° C. for 3 h. After cooling, the reaction mixture was diluted with 50 ml EA and washed three times with 20 ml NaHCO₃ (aq. sat), dried over Na₂SO₄, filtered and evaporated to dryness. The crude product was purified by FC on silica gel using a gradient of heptane and EA, and the fractions containing pure product were combined and evaporated to afford 183 mg of the title compound. LC/MS: m/z=303.2 [M+H]⁺; RT: 1.68 min (LC/MS-method A).

### Step 2: 4-((1,4-dimethyl-1H-pyrazol-5-yl)methyl)pyridin-2-amine

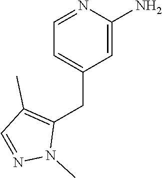

182 mg tert-butyl (4-((1,4-dimethyl-1H-pyrazol-5-yl)methyl)pyridin-2-yl)carbamate was dissolved in 5 ml dioxane, and 1.50 ml 4N HCl in dioxane was added and the mixture stirred for 16 h at ambient temperature. Then another 1.50 ml 4N HCl in dioxane was added, and the mixture stirred for another 6 h at ambient temperature before the reaction mixture was diluted with 20 ml DCM and washed 3 times with Na₂CO₃ solution (aq, sat), dried over Na₂SO₄, filtered and evaporated to dryness. The crude product (Intermediate 36) was used without further purification. LC/MS: m/z=203.1 [M+H]⁺; RT: 0.66 min (LC/MS-method A).

### Intermediate 37: 2-amino-2-(4,4-difluorocyclohexyl)-N-(4-((1-methyl-6-oxo-1,6-dihydropyridin-3-yl)methyl)pyridin-2-yl)acetamide

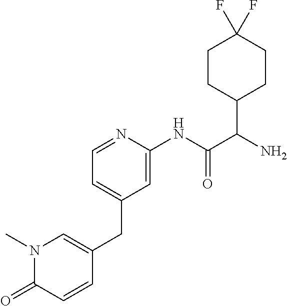 [SMILES: Cn1cc(Cc2ccnc(NC(=O)C(N)C3CCC(F)(F)CC3)c2)ccc1=O]

The title compound was prepared by the same procedure as used for 2-amino-2-(4,4-difluorocyclohexyl)-N-(4-((1-methyl-6-oxo-1,6-dihydropyridin-2-yl)methyl)pyridin-2-yl)acetamide [Intermediate 33]. LC/MS: m/z=391.1 [M+H]⁺; RT: 0.81 min (LC/MS-method A).

### Intermediate 38: 2-amino-2-(4,4-difluorocyclohexyl)-N-(4-((1,4-dimethyl-1H-pyrazol-5-yl)methyl)pyridin-2-yl)acetamide

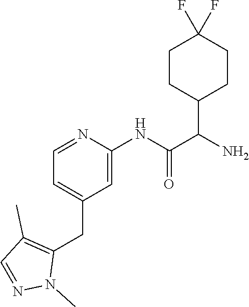 [SMILES: Cc1cnn(C)c1Cc1ccnc(NC(=O)C(N)C2CCC(F)(F)CC2)c1]

### Step 1: tert-butyl (1-(4,4-difluorocyclohexyl)-2-((4-((1,4-dimethyl-1H-pyrazol-5-yl)methyl)pyridin-2-yl)amino)-2-oxoethyl)carbamate

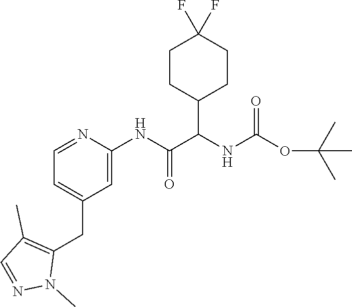 [SMILES: Cc1cnn(C)c1Cc1ccnc(NC(=O)C(NC(=O)OC(C)(C)C)C2CCC(F)(F)CC2)c1]

The title compound was prepared using the procedure described for the preparation of tert-butyl ((S)-2-((4-((1,4-dimethyl-1H-pyrazol-5-yl)methyl)pyridin-2-yl)amino)-1-((1r,4S)-4-methylcyclohexyl)-2-oxoethyl)carbamate [Example 053] to afford 72 mg of product. LC/MS: m/z=491.1 [M+H]⁺; RT: 1.86 min (LC/MS-method A).

### Step 2: 2-amino-2-(4,4-difluorocyclohexyl)-N-(4-((1,4-dimethyl-1H-pyrazol-5-yl)methyl)pyridin-2-yl)acetamide HCl salt

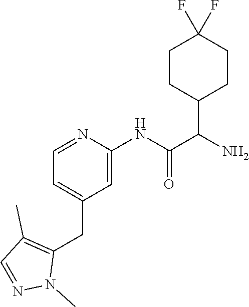 [SMILES: Cc1cnn(C)c1Cc1ccnc(NC(=O)C(N)C2CCC(F)(F)CC2)c1]

Using the procedure described for Intermediate 33, 48 mg of the title compound (Intermediate 38) was obtained and used without further purification. LC/MS: m/z=378.3 [M+H]⁺; RT: 0.94 min (LC/MS-method B).

### Intermediate 39: 2-amino-2-(4,4-difluorocyclohexyl)-N-(4-((1-methyl-2-oxo-1,2-dihydropyridin-4-yl)methyl)pyridin-2-yl)acetamide

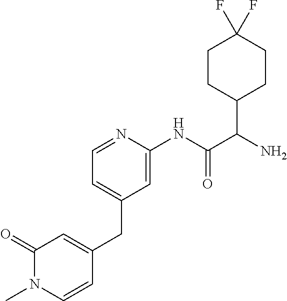 [SMILES: Cn1ccc(Cc2ccnc(NC(=O)C(N)C3CCC(F)(F)CC3)c2)cc1=O]

### Step 1: N-(1-(4,4-difluorocyclohexyl)-2-((4-((1-methyl-2-oxo-1,2-dihydropyridin-4-yl)methyl)pyridin-2-yl)amino)-2-oxoethyl)-1-methyl-1H-pyrazole-5-carboxamide

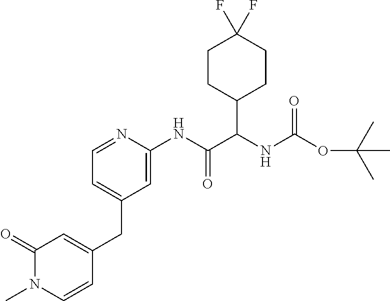 [SMILES: Cn1ccc(Cc2ccnc(NC(=O)C(NC(=O)OC(C)(C)C)C3CCC(F)(F)CC3)c2)cc1=O]

The title compound was prepared using the procedure described for tert-butyl ((S)-2-((4-((1,4-dimethyl-1H-pyrazol-5-yl)methyl)pyridin-2-yl)amino)-1-((1r,4S)-4-methylcyclohexyl)-2-oxoethyl)carbamate (Example 53) to afford 76 mg of product. LC/MS: m/z=491.3 [M+H]⁺; RT: 1.87 min (LC/MS-method A).

### Step 2: 2-amino-2-(4,4-difluorocyclohexyl)-N-(4-((1-methyl-2-oxo-1,2-dihydropyridin-4-yl)methyl)pyridin-2-yl)acetamide

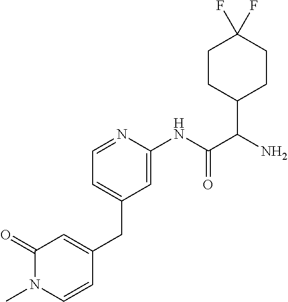 [SMILES: Cn1ccc(Cc2ccnc(NC(=O)C(N)C3CCC(F)(F)CC3)c2)cc1=O]

Using the procedure described for Intermediate 33, with 73 mg of N-(1-(4,4-difluorocyclohexyl)-2-((4-((1-methyl-2-oxo-1,2-dihydropyridin-4-yl)methyl)pyridin-2-yl)amino)-2-oxoethyl)-1-methyl-1H-pyrazole-5-carboxamide, 55 mg of the title compound (Intermediate 39) was obtained and used without further purification. LC/MS: m/z=378.3 [M+H]⁺; RT: 0.94 min (LC/MS-method B).

### Intermediate 40: (S)-2-amino-N-(4-((1,4-dimethyl-1H-pyrazol-5-yl)methyl)pyridin-2-yl)-2-((1r,4S)-4-methylcyclohexyl)acetamide HCl salt

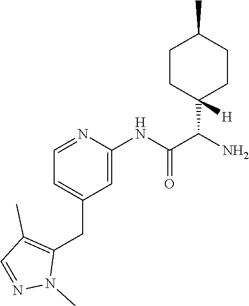 [SMILES: Cc1cnn(C)c1Cc1ccnc(NC(=O)[C@@H](N)[C@H]2CC[C@H](C)CC2)c1]

Using the procedure described for Intermediate 33, using 59 mg of tert-butyl ((S)-2-((4-((1,4-dimethyl-1H-pyrazol-5-yl)methyl)pyridin-2-yl)amino)-1-((1r,4S)-4-methylcyclohexyl)-2-oxoethyl)carbamate (Example 53), 46 mg of the title compound was obtained and used without further purification. LC/MS: m/z=356.3 [M+H]⁺; RT: 1.40 min (LC/MS-method A).

### Intermediate 41: N-((2-((S)-2-amino-2-(4,4-difluorocyclohexyl)acetamido)pyridin-4-yl)(cyclopropyl)methyl)-4,4,4-trifluorobutanamide

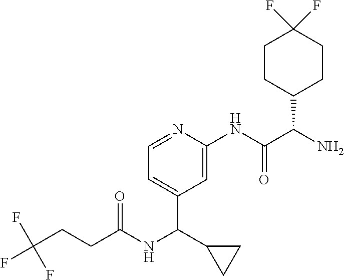 [SMILES: N[C@H](C(=O)Nc1cc(C(NC(=O)CCC(F)(F)F)C2CC2)ccn1)C1CCC(F)(F)CC1]

The title compound was prepared following the procedure described for N-((2-(2-amino-2-(4,4-difluorocyclohexyl)acetamido)pyridin-4-yl)(cyclopropyl)methyl)-4,4,4-trifluorobutanamide (Intermediate 35 diastereomer 1), using 375 mg of tert-butyl ((1S)-2-((4-(cyclopropyl(4,4,4-trifluorobutanamido)methyl)pyridin-2-yl)amino)-1-(4,4-difluorocyclohexyl)-2-oxoethyl)carbamate diastereomer 1 (Example 72), to afford 275 mg title compound that was used without further purification. LC/MS: m/z=461.3 [M+H]⁺; RT: 1.33 min (LC/MS-method A).

### Intermediate 42: N-((2-((S)-2-amino-2-((1r,4S)-4-methylcyclohexyl)acetamido)-pyridin-4-yl)(cyclopropyl)methyl)-4,4,4-trifluorobutanamide

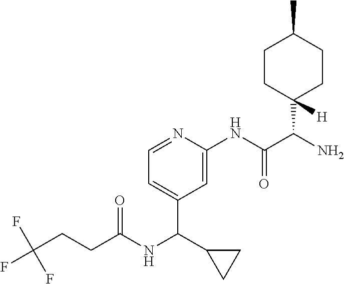 [SMILES: C[C@H]1CC[C@H]([C@H](N)C(=O)Nc2cc(C(NC(=O)CCC(F)(F)F)C3CC3)ccn2)CC1]

The title compound was prepared following the procedure described for N-((2-(2-amino-2-(4,4-difluorocyclohexyl)acetamido)pyridin-4-yl)(cyclopropyl)methyl)-4,4,4-trifluorobutanamide (Intermediate 35 diastereomer 1), using 130 mg of tert-butyl ((1S)-2-((4-(cyclopropyl(4,4,4-trifluorobutanamido)methyl)pyridin-2-yl)amino)-1-((1r,4S)-4-methylcyclohexyl)-2-oxoethyl)carbamate diastereomer 1 (Example 71), to afford 100 mg title compound that was used without further purification. LC/MS: m/z=441.2 [M+H]⁺; RT: 1.00 min (LC/MS-method B).

### Intermediate 43: (2S)-2-amino-2-((1r,4S)-4-methylcyclohexyl)-N-(4-((2-oxopyrrolidin-3-yl)oxy)pyridin-2-yl)acetamide

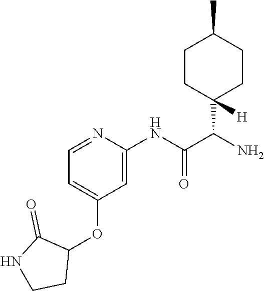 [SMILES: C[C@H]1CC[C@H]([C@H](N)C(=O)Nc2cc(OC3CCNC3=O)ccn2)CC1]

### Step 1: tert-butyl (4-((2-oxopyrrolidin-3-yl)oxy)pyridin-2-yl)carbamate

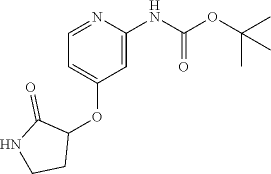 [SMILES: CC(C)(C)OC(=O)Nc1cc(OC2CCNC2=O)ccn1]

3-bromopyrrolidin-2-one (491 mg) was dissolved in 8 ml dry DMF, and 420 mg tert-Butyl N-(4-hydroxy-2-pyridyl)carbamate and 828 mg potassium carbonate were added, and the mixture was heated to 80° C. under stirring for 6 h. After cooling the mixture was rotated to dryness, 20 ml water added, and the mixture stirred for 30 min. Then the product was filtered off, washed with water, and dried under vacuum. This afforded 250 mg of the title product that was used directly in the next step. LC/MS: m/z=294.1 [M+H]⁺; RT: 1.62 min (LC/MS-method A).

### Step 2: 3-((2-aminopyridin-4-yl)oxy)pyrrolidin-2-one HCl Salt

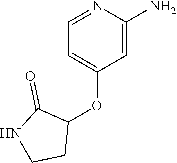 [SMILES: Nc1cc(OC2CCNC2=O)ccn1]

Tert-butyl (4-((2-oxopyrrolidin-3-yl)oxy)pyridin-2-yl)carbamate (300 mg) was dissolved in 10 ml dry dioxane and 2.6 ml 4 N HCl in dioxane was added, and the mixture stirred for 16 h at ambient temperature. Then another 1.3 ml 4 N HCl in dioxane was added, and the mixture stirred for another 6 h at ambient temperature before addition of 15 ml MTBE to precipitate the product. The product was filtered off and dried under vacuum to yield 215 mg of the title product that was used directly in the next step. LC/MS: m/z=194.1 [M+H]⁺; rt: 0.18 min (LC/MS-method B).

### Step 3: Tert-butyl ((1S)-1-((1r,4S)-4-methylcyclohexyl)-2-oxo-2-((4-((2-oxopyrrolidin-3-yl)oxy)pyridin-2-yl)amino)ethyl)carbamate

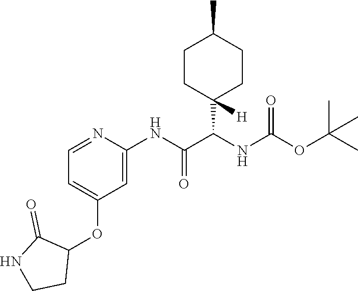 [SMILES: CC(C)(C)OC(=O)N[C@H](C(=O)Nc1cc(OC2CCNC2=O)ccn1)[C@H]1CC[C@H](C)CC1]

Following the procedure described for Intermediate 34—step 6, using 220 mg of (S)-2-((tert-butoxycarbonyl)amino)-2-((1r,4S)-4-methylcyclohexyl)acetic acid and 186 mg of 3-((2-aminopyridin-4-yl)oxy)pyrrolidin-2-one HCl salt, afforded 400 mg of the title compound that was used directly in the next step. LC/MS: m/z=447.2 [M+H]⁺; rt: 1.11 min (LC/MS-method B).

### Step 4: (2S)-2-amino-2-((1r,4S)-4-methylcyclohexyl)-N-(4-((2-oxopyrrolidin-3-yl)oxy)pyridin-2-yl)acetamide

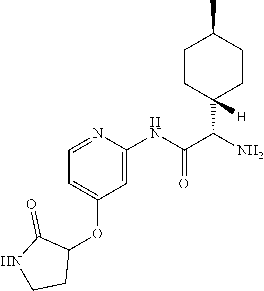 [SMILES: C[C@H]1CC[C@H]([C@H](N)C(=O)Nc2cc(OC3CCNC3=O)ccn2)CC1]

Following the procedure described for Intermediate 34—step 7, tert-butyl ((1S)-1-((1r,4S)-4-methylcyclohexyl)-2-oxo-2-((4-((2-oxopyrrolidin-3-yl)oxy)pyridin-2-yl)amino)-ethyl)carbamate (360 mg) afforded 105 mg of the title compound (Intermediate 43) that was used without further purification. LC/MS: m/z=347.3 [M+H]⁺; rt: 0.81 min (LC/MS-method B).

### Intermediate 44: Tert-butyl ((1S)-2-((4-((((S)-2-amino-3,3,3-trifluoropropyl)amino)-methyl-d)pyridin-2-yl)amino)-1-((1r,4S)-4-methylcyclohexyl)-2-oxoethyl)carbamate

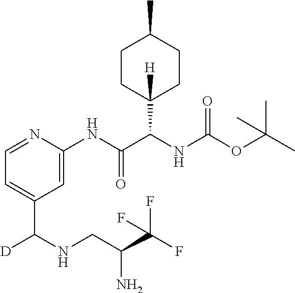

Following the procedure of Intermediate 1, and using NaBD₃CN as reducing reagent, 96 mg (61%) of the title compound was obtained. LC/MS: m/z=489.3 [M+H]⁺; rt: 1.70 min (LC/MS-method A).

### Intermediate 45, DS1: N-(1-(2-((S)-2-amino-2-(4,4-difluorocyclohexyl)acetamido)-pyridin-4-yl)-2-methoxyethyl)-4,4,4-trifluorobutanamide diastereomer 1

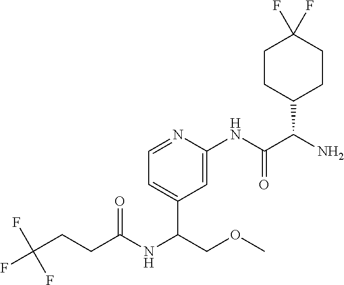 [SMILES: COCC(NC(=O)CCC(F)(F)F)c1ccnc(NC(=O)[C@@H](N)C2CCC(F)(F)CC2)c1]

Using the procedure described for Intermediate 33, using 340 mg of tert-butyl ((1S)-1-(4,4-difluorocyclohexyl)-2-((4-(2-methoxy-1-(4,4,4-trifluorobutanamido)ethyl)-pyridin-2-yl)amino)-2-oxoethyl)carbamate diastereomer 1 (Example 131), 265 mg of the title compound was obtained and used without further purification. LC/MS: m/z=467.2 [M+H]⁺; RT: 0.88 min (LC/MS-method B).

### Intermediate 45, DS2: N-(1-(2-((S)-2-amino-2-(4,4-difluorocyclohexyl)acetamido)-pyridin-4-yl)-2-methoxyethyl)-4,4,4-trifluorobutanamide diastereomer 2

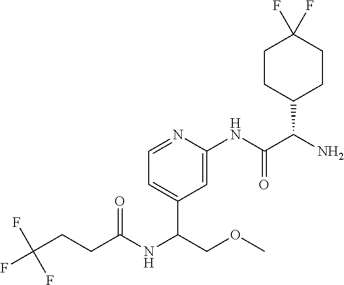 [SMILES: COCC(NC(=O)CCC(F)(F)F)c1ccnc(NC(=O)[C@@H](N)C2CCC(F)(F)CC2)c1]

Prepared analogously to Intermediate 45 DS1, using Example 132 as starting material. LC/MS: m/z=467.2 [M+H]⁺; RT: 0.88 min (LC/MS-method B).

### Intermediate 46: (S)-2-amino-2-((1r,4S)-4-methylcyclohexyl)-N-(4-(((3R,5S)-2-oxo-5-(trifluoromethyl)pyrrolidin-3-yl)methyl)pyridin-2-yl)acetamide

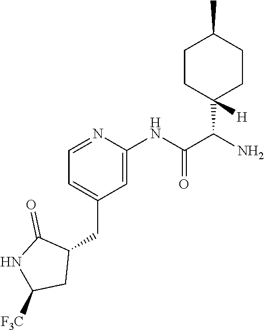 [SMILES: C[C@H]1CC[C@H]([C@H](N)C(=O)Nc2cc(C[C@@H]3C[C@@H](C(F)(F)F)NC3=O)ccn2)CC1]

### Step 1: dimethyl (R,E)-2-(2-((tert-butylsulfinyl)imino)ethyl)malonate

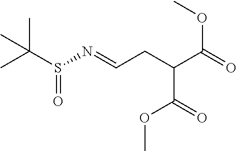 [SMILES: COC(=O)C(C/C=N/[S@](=O)C(C)(C)C)C(=O)OC]

To a solution of dimethyl 2-(2-oxoethyl)malonate (33 g, 189.49 mmol, 1 eq) and (R)-2-methylpropane-2-sulfinamide (29.86 g, 246.34 mmol, 1.3 eq) in DCM (350 ml) was added pyrrolidine (1.35 g, 18.95 mmol, 1.58 mL, 0.1 eq). The mixture was stirred at 25° C. for 12 hr before removal of the solvent under reduced pressure to afford the crude product which was purified by FC on silica gel using PE/EA. This afforded 27.73 g of the title compound. ¹H NMR (400 MHz, CDCl₃) δ ppm: 8.12 (t, J=2.7 Hz, 1H), 3.98 (dd, J=6.1, 8.1 Hz, 1H), 3.76 (d, J=4.0 Hz, 6H), 3.25-3.10 (m, 2H), 1.16 (s, 9H).

### Step 2: dimethyl 2-((S)-2-(((R)-tert-butylsulfinyl)amino)-3,3,3-trifluoropropyl)malonate

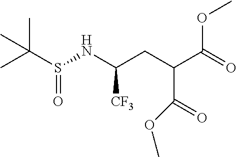 [SMILES: COC(=O)C(C[C@H](N[S@](=O)C(C)(C)C)C(F)(F)F)C(=O)OC]

To a solution of dimethyl (R,E)-2-(2-((tert-butylsulfinyl)imino)ethyl)malonate (27.7 g, 99.88 mmol, 1 eq) and tetrabutylammoniumacetate (36.14 g, 119.85 mmol, 36.50 ml, 1.2 eq) in THE (554 ml) was added TMS-CF₃ (35.51 g, 249.70 mmol, 2.5 eq) at −55° C. and stirred at −55° C. for 1 h before the cooling was removed and the mixture allowed to warm to ambient temperature. After 3 h the reaction mixture was quenched by aqueous ammonium chloride (100 ml). The mixture was diluted with water (1200 ml) and extracted with EA (3×500 ml), and the organic layer was washed with brine (3×500 ml) before being dried over anhydrous Na₂SO₄ and then concentrated under reduced pressure to afford the crude product. Purification by FC on silica gel using PE/EA afforded 12 g of the title compound as a yellow solid after removal of the solvents by rotary evaporation. LC/MS: m/z=348.1 [M+H]⁺; rt: 0.35 min. ¹H NMR (400 MHz, CDCl₃) δ ppm: 3.84 (dd, J=7.1, 13.4 Hz, 2H), 3.79 (d, J=6.5 Hz, 6H), 2.43 (ddd, J=3.1, 7.7, 14.8 Hz, 1H), 2.17 (ddd, J=6.1, 11.3, 14.8 Hz, 1H), 1.24 (s, 9H).

### Step 3: dimethyl 2-((2-((tert-butoxycarbonyl)amino)pyridin-4-yl)methyl)-2-((S)-2-(((R)-tert-butylsulfinyl)amino)-3,3,3-trifluoropropyl)malonate

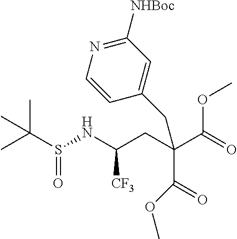 [SMILES: COC(=O)C(Cc1ccnc(NC(=O)OC(C)(C)C)c1)(C[C@H](N[S@](=O)C(C)(C)C)C(F)(F)F)C(=O)OC]

To a solution of dimethyl 2-((S)-2-(((R)-tert-butylsulfinyl)amino)-3,3,3-trifluoropropyl)malonate (12 g, 34.55 mmol, 1 eq) in DMF (180 mL) was added Cs₂CO₃ (11.26 g, 34.55 mmol, 1 eq), KI (1.15 g, 6.91 mmol, 0.2 eq) and tert-butyl (4-(bromomethyl)pyridin-2-yl)carbamate (9.92 g, 34.55 mmol, 1 eq). The mixture was stirred at 25° C. for 11 hr. The reaction mixture was diluted with water (600 mL) at 0° C. and extracted with EA (3×150 ml) and the combined organic layers were washed with brine (2×150 ml), dried over anhydrous Na₂SO₄, filtered and concentrated under reduced pressure to give the crude product that was purified by FC on silica gel using PE/EA to afford 16.53 g of the title compound as a colorless solid. LC/MS: m/z=554.2 [M+H]⁺; rt: 0.76 min. ¹H NMR (400 MHz, CDCl₃) δ ppm: 8.16 (d, J=5.1 Hz, 1H), 8.14-8.11 (m, 1H), 7.81 (s, 1H), 6.75 (dd, J=1.1, 5.1 Hz, 1H), 4.37 (d, J=5.1 Hz, 1H), 4.03-3.92 (m, 1H), 3.86 (d, J=6.8 Hz, 6H), 3.63-3.18 (m, 2H), 2.34 (dd, J=10.8, 15.3 Hz, 1H), 2.19 (dd, J=2.3, 15.3 Hz, 1H), 1.52 (s, 9H), 1.21 (s, 9H).

### Step 4: methyl (5S)-3-((2-((tert-butoxycarbonyl)amino)pyridin-4-yl)methyl)-2-oxo-5-(trifluoromethyl)pyrrolidine-3-carboxylate

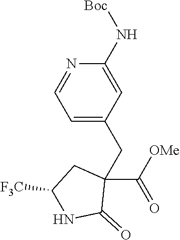 [SMILES: COC(=O)C1(Cc2ccnc(NC(=O)OC(C)(C)C)c2)C[C@@H](C(F)(F)F)NC1=O]

To a solution of dimethyl 2-((2-((tert-butoxycarbonyl)amino)pyridin-4-yl)methyl)-2-((S)-2-(((R)-tert-butylsulfinyl)amino)-3,3,3-trifluoropropyl)malonate (27.8 g, 50.22 mmol, 1 eq) in MeOH (280 ml) was added HCl/dioxane (4 M, 25.11 ml, 2 eq). The reaction mixture was stirred at 20° C. for 2 hr and then at 40° C. for 2 hr before being evaporated under reduced pressure to remove the volatiles. Then the crude product was diluted with DCM (200 ml) and Na₂CO₃ solution was added at 0° C. until pH 7. The mixture was extracted with DCM (3×200 ml and the combined organic layers were washed with brine (2×200 ml) before being dried over anhydrous Na₂SO₄, filtered, and concentrated under reduced pressure to afford 22.28 g of the title compound as a yellow oil that was used directly in the next step without further purification. LC/MS: m/z=418.1 [M+H]⁺; rt: 0.62 min.

### Step 5: (5S)-3-((2-((tert-butoxycarbonyl)amino)pyridin-4-yl)methyl)-2-oxo-5-(trifluoromethyl)pyrrolidine-3-carboxylic acid

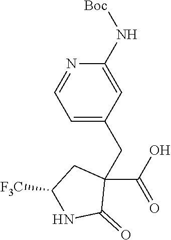 [SMILES: CC(C)(C)OC(=O)Nc1cc(CC2(C(=O)O)C[C@@H](C(F)(F)F)NC2=O)ccn1]

To a solution of methyl (5S)-3-((2-((tert-butoxycarbonyl)amino)pyridin-4-yl)methyl)-2-oxo-5-(trifluoromethyl)pyrrolidine-3-carboxylate (22.2 g, 53.19 mmol, 1 eq) in EtOH (440 ml) and H₂O (440 ml) was added LiOH·H₂O (5.58 g, 132.97 mmol, 2.5 eq). The reaction mixture was stirred at 25° C. for 6 hr and diluted with water (500 ml) and extracted with PE:EA 9:1 (2×300 ml). The aqueous phase was adjusted to pH~7 and extracted with DCM (3×400 ml) and the combined organic layers were washed with brine (2×200 ml), dried over anhydrous Na₂SO₄, filtered and concentrated under reduced pressure to give a residue to afford 18.2 g of the title compound as a colourless solid that was used directly in the next step without further purification. LC/MS: m/z=404.1 [M+H]⁺; rt: 0.44 and 0.46 min.

### Step 6: tert-butyl (4-(((3R,5S)-2-oxo-5-(trifluoromethyl)pyrrolidin-3-yl)methyl)pyridin-2-yl)carbamate and tert-butyl (4-(((3S,5S)-2-oxo-5-(trifluoromethyl)pyrrolidin-3-yl)methyl)pyridin-2-yl)carbamate

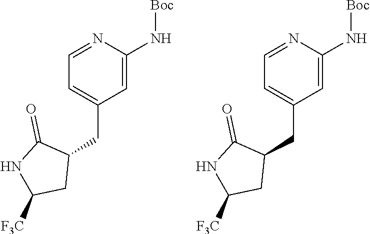 [SMILES: CC(C)(C)OC(=O)Nc1cc(C[C@@H]2C[C@@H](C(F)(F)F)NC2=O)ccn1 ; CC(C)(C)OC(=O)Nc1cc(C[C@H]2C[C@@H](C(F)(F)F)NC2=O)ccn1]

To a solution of (5S)-3-((2-((tert-butoxycarbonyl)amino)pyridin-4-yl)methyl)-2-oxo-5-(trifluoromethyl)pyrrolidine-3-carboxylic acid (18.2 g, 45.12 mmol, 1 eq) in DMSO (180 ml) was added LiCl (3.83 g, 90.24 mmol, 1.85 mL, 2 eq) and H₂O (3.25 g, 180.49 mmol, 3.25 ml, 4 eq). The mixture was stirred at 100° C. for 1.5 hr. After cooling, the reaction mixture was diluted with water (600 ml) and extracted with EA (3×300 ml) and the combined organic layers were washed with brine (3×500 ml), dried over anhydrous Na₂SO₄, filtered and concentrated under reduced pressure to afford the crude product. Purification by FC using PE/EA afforded:

781 mg of tert-butyl (4-(((3R,5S)-2-oxo-5-(trifluoromethyl)pyrrolidin-3-yl)methyl-)pyridin-2-yl)carbamate as a colourless solid. LC/MS: m/z=360.1 [M+H]⁺; rt: 0.61 min. ¹H NMR (400 MHz, CDCl₃) δ ppm: 8.19 (d, J=5.3 Hz, 1H), 8.09 (s, 1H), 7.84 (s, 1H), 6.85 (d, J=5.1 Hz, 1H), 6.31 (br s, 1H), 3.95 (quin, J=7.6 Hz, 1H), 3.24 (dd, J=4.2, 13.8 Hz, 1H), 2.92 (dq, J=4.4, 9.4 Hz, 1H), 2.66 (dd, J=9.6, 13.8 Hz, 1H), 2.31 (dd, J=9.1, 14.0 Hz, 1H), 2.11 (td, J=9.6, 14.1 Hz, 1H), 1.54 (s, 9H); and

3.93 g of tert-butyl (4-(((3S,5S)-2-oxo-5-(trifluoromethyl)pyrrolidin-3-yl)methyl)-pyridin-2-yl)carbamate as a colourless solid. LC/MS: m/z=360.1 [M+H]⁺; rt: 0.61 min. ¹H NMR (400 MHz, CDCl₃) δ ppm: 8.51 (s, 1H), 8.20 (d, J=5.3 Hz, 1H), 7.87 (s, 1H), 6.83 (dd, J=1.1, 5.1 Hz, 1H), 6.62 (s, 1H), 4.13-4.03 (m, 1H), 3.31 (dd, J=4.1, 14.3 Hz, 1H), 2.94-2.83 (m, 1H), 2.66 (dd, J=10.6, 14.2 Hz, 1H), 2.49-2.38 (m, 1H), 1.86 (ddd, J=6.9, 8.3, 13.9 Hz, 1H), 1.55 (s, 9H).

### Step 7: (3R,5S)-3-((2-aminopyridin-4-yl)methyl)-5-(trifluoromethyl)pyrrolidin-2-one HCl Salt

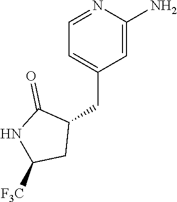 [SMILES: Nc1cc(C[C@@H]2C[C@@H](C(F)(F)F)NC2=O)ccn1]

Following the procedure for Boc deprotection described for 3-((2-aminopyridin-4-yl)oxy)pyrrolidin-2-one (Intermediate 43, step 2), using 360 mg of tert-butyl (4-(((3R,5S)-2-oxo-5-(trifluoromethyl)pyrrolidin-3-yl)methyl)pyridin-2-yl)carbamate, 255 mg of the title compound was obtained and used directly in the next step. LC/MS: m/z=260.1 [M+H]⁺; rt: 0.56 min (LC/MS-method A).

### Step 8: tert-butyl ((S)-1-((1r,4S)-4-methylcyclohexyl)-2-oxo-2-((4-(((3R,5S)-2-oxo-5-(trifluoromethyl)pyrrolidin-3-yl)methyl)pyridin-2-yl)amino)ethyl)carbamate

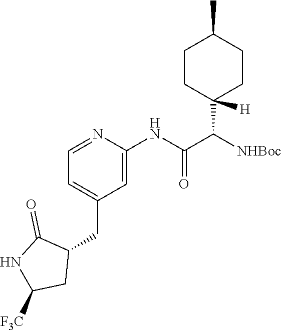 [SMILES: CC(C)(C)OC(=O)N[C@H](C(=O)Nc1cc(C[C@@H]2C[C@@H](C(F)(F)F)NC2=O)ccn1)[C@H]1CC[C@H](C)CC1]

230 mg of (2S)-2-(tert-butoxycarbonylamino)-2-(4-methylcyclohexyl)acetic acid was dissolved in 20 ml DCM and 612 mg TBTU added together with 0.89 ml DIPEA and the mixture was stirred for 30 min at ambient temperature before addition of 250 mg of (3R,5S)-3-((2-aminopyridin-4-yl)methyl)-5-(trifluoromethyl)pyrrolidin-2-one HCl salt. After stirring for 16 h at ambient temperature 50 ml DCM was added the mixture washed with 30 ml water and 30 ml NaCl solution, dried over Na₂SO₄, filtered and evaporated to dryness. The crude product was purified by FC on silica using MeOH:DCM as the eluent to afford 210 mg of the title compound. LC/MS: m/z=513.4 [M+H]⁺; rt: 2.37 min (LC/MS-method A).

### Step 9: (S)-2-amino-2-((1r,4S)-4-methylcyclohexyl)-N-(4-(((3R,5S)-2-oxo-5-(trifluoromethyl)pyrrolidin-3-yl)methyl)pyridin-2-yl)acetamide

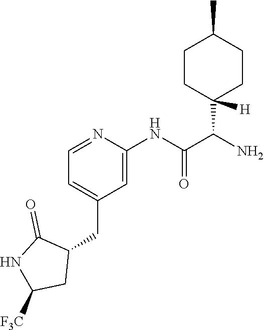 [SMILES: C[C@H]1CC[C@H]([C@H](N)C(=O)Nc2cc(C[C@@H]3C[C@@H](C(F)(F)F)NC3=O)ccn2)CC1]

210 mg of tert-butyl ((S)-1-((1r,4S)-4-methylcyclohexyl)-2-oxo-2-((4-(((3R,5S)-2-oxo-5-(trifluoromethyl)pyrrolidin-3-yl)methyl)pyridin-2-yl)amino)ethyl)carbamate was dissolved in 30 ml DCM and 420 mg TsOH was added and the mixture stirred for 16 h at ambient temperature. The solvent was removed by rotary evaporation and residue dissolved in 4.1 ml 0.5M NaOH solution and extracted with DCM (4×10 ml). The combined organic phases were dried over Na₂SO₄, filtered and evaporated to dryness to afford 110 mg of the title compound (Intermediate 46) that was used without further purification. LC/MS: m/z=413.2 [M+H]⁺; rt: 0.92 min (LC/MS-method B).

### Intermediate 47: (S)-2-amino-2-((1r,4S)-4-methylcyclohexyl)-N-(4-(((3S,5S)-2-oxo-5-(trifluoromethyl)pyrrolidin-3-yl)methyl)pyridin-2-yl)acetamide

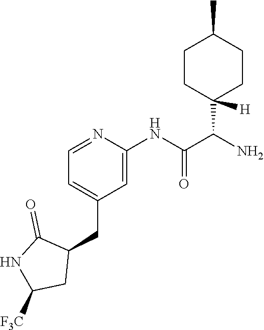 [SMILES: C[C@H]1CC[C@H]([C@H](N)C(=O)Nc2cc(C[C@H]3C[C@@H](C(F)(F)F)NC3=O)ccn2)CC1]

The title compound was prepared analogously to (S)-2-amino-2-((1r,4S)-4-methylcyclohexyl)-N-(4-(((3R,5S)-2-oxo-5-(trifluoromethyl)pyrrolidin-3-yl)methyl)pyridin-2-yl)acetamide (Intermediate 46) using tert-butyl (4-(((3S,5S)-2-oxo-5-(trifluoromethyl)pyrrolidin-3-yl)methyl)pyridin-2-yl)carbamate from step 7 onwards. LC/MS: m/z=413.2 [M+H]⁺; rt: 0.95 min (LC/MS-method B).

### Intermediate 48-DS1: (2S)-2-Amino-N-(4-((R or S)-cyclopropyl((S)-2-oxo-4-(trifluoromethyl)imidazolidin-1-yl)methyl)pyridin-2-yl)-2-(4,4-difluorocyclohexyl)-acetamide hydrochloride—Diastereomer 1

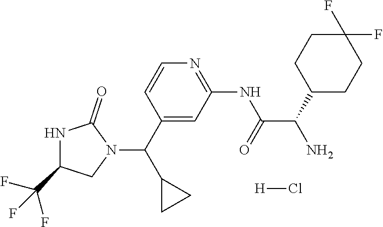

### Step 1: Tert-butyl ((1S)-2-((4-(cyclopropyl(hydroxy)methyl)pyridin-2-yl)amino)-1-(4,4-difluorocyclohexyl)-2-oxoethyl)carbamate

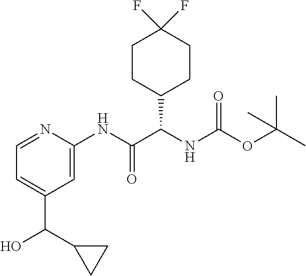 [SMILES: CC(C)(C)OC(=O)N[C@H](C(=O)Nc1cc(C(O)C2CC2)ccn1)C1CCC(F)(F)CC1]

To a solution of tert-butyl (S)-(1-(4,4-difluorocyclohexyl)-2-((4-formylpyridin-2-yl)amino)-2-oxoethyl)carbamate (Int. 3, Step 3; 2 g) in THE (30 ml) was added bromo(cyclopropyl)magnesium (1 M, 22.7 ml) at 0° C. The mixture was stirred at 20° C. for 20 min. Then the reaction mixture was quenched by the addition of NH₄Cl solution at 0° C., and then diluted with water (100 ml) and extracted with EA (50 ml, 3×). The combined organic layers were washed with brine (100 ml, 2×), dried over anhydrous Na₂SO₄, filtered and concentrated under reduced pressure to give a residue, which was purified by flash silica gel chromatography (ISCO®; 40 g SepaFlash® silica flash column, eluent of 0~30~50~80% EA (10% DCM)/PE gradient; flow 80 ml/min) to yield 1.64 g of the title compound. LC/MS: m/z=440.3 [M+H]⁺; rt: 0.36 min (LC/MS-method C).

### Step 2: Tert-butyl (S)-(2-((4-(cyclopropanecarbonyl)pyridin-2-yl)amino)-1-(4,4-difluorocyclohexyl)-2-oxoethyl)carbamate

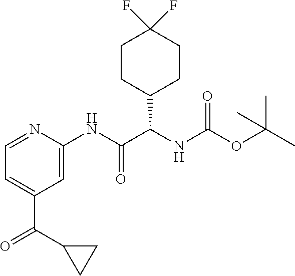 [SMILES: CC(C)(C)OC(=O)N[C@H](C(=O)Nc1cc(C(=O)C2CC2)ccn1)C1CCC(F)(F)CC1]

To a solution of tert-butyl ((1S)-2-((4-(cyclopropyl(hydroxy)methyl)pyridin-2-yl)amino)-1-(4,4-difluorocyclohexyl)-2-oxoethyl)carbamate (1.64 g) in DCM (20 ml) was added DMP (1.90 g) at 0° C. The mixture was stirred at 20° C. for 1.5 h. Then the reaction mixture was quenched by addition NH₄Cl solution at 0° C. and diluted with water (100 ml) and extracted with EA (50 ml, 3×). The combined organic layers were washed with brine (100 ml, 2×), dried over anhydrous Na₂SO₄, filtered and concentrated under reduced pressure to give a residue, which was purified by flash silica gel chromatography (ISCO®; 40 g SepaFlash® silica flash column, eluent of 0~30~50~80% EA (10% DCM)/PE gradient; flow 80 ml/min) to yield 1.42 g of the title compound. LC/MS: m/z=438.2 [M+H]⁺; rt: 0.47 min (LC/MS-method C).

### Step 3: Tert-butyl ((1S)-2-((4-((((S)-2-amino-3,3,3-trifluoropropyl)amino)(cyclopropyl)-methyl)pyridin-2-yl)amino)-1-(4,4-difluorocyclohexyl)-2-oxoethyl)carbamate

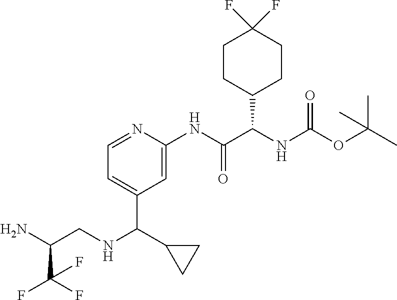 [SMILES: CC(C)(C)OC(=O)N[C@H](C(=O)Nc1cc(C(NC[C@H](N)C(F)(F)F)C2CC2)ccn1)C1CCC(F)(F)CC1]

To a solution of tert-butyl (S)-(2-((4-(cyclopropanecarbonyl)pyridin-2-yl)amino)-1-(4,4-difluorocyclohexyl)-2-oxoethyl)carbamate (1.27 g) in IPA (15 ml) was added TEA (1.21 ml) and (S)-3,3,3-trifluoropropane-1,2-diamine dihydrochloride (I-001-B6, 875 mg). The mixture was stirred at 80° C. for 16 h. Then the mixture was dissolved in MeOH (15 ml), NaBH₃CN (547 mg) was added and the mixture was stirred at 25° C. for 10 min, then AcOH (1.66 ml) was added dropwise and stirred at 30° C. for 30 min. The reaction mixture was dissolved in DCM (50 ml) and water (150 ml) and then adjusted to pH to 7~8 with NaHCO₃. The organic layer was separated and the aqueous phase was extracted with DCM (50 ml, 2×). The combined organic layers were washed with brine (50 ml, 3×) and dried over anhydrous Na₂SO₄, filtered and concentrated to yield 1.6 g of the crude title compound. LC/MS: m/z=550.3 [M+H]⁺; rt: 0.35 min (LC/MS-method C).

### Step 4-P1 and -P2: Tert-butyl ((1S)-2-((4-((R or S)-cyclopropyl((S)-2-oxo-4-(trifluoromethyl)imidazolidin-1-yl)methyl)pyridin-2-yl)amino)-1-(4,4-difluorocyclohexyl)-2-oxoethyl)carbamate

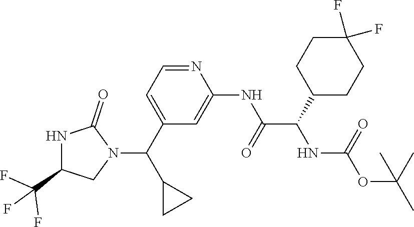 [SMILES: CC(C)(C)OC(=O)N[C@H](C(=O)Nc1cc(C(C2CC2)N2C[C@@H](C(F)(F)F)NC2=O)ccn1)C1CCC(F)(F)CC1]

To a solution of tert-butyl ((1S)-2-((4-((((S)-2-amino-3,3,3-trifluoropropyl)amino)(cyclopropyl) methyl)pyridin-2-yl)amino)-1-(4,4-difluorocyclohexyl)-2-oxoethyl)carbamate (1.6 g) in THE (50 ml) was added CDI (1.42 g) at 65° C. The mixture was stirred at 65° C. for 1 h. Then additional CDI (1.42 g) was added at 65° C. and stirring was continued at 65° C. for 0.5 h. Then NaOH solution (2 M, 20 ml) was added at 25° C. and the mixture was stirred at 25° C. for 10 min, then heated to 65° C. and stirred at 65° C. for 5~10 min. After cooling he mixture was concentrated under reduced pressure to remove THE and diluted with EA:DCM (200 ml, 10:1) and washed with brine (100 ml, 4×). The organic layer was dried over anhydrous Na₂SO₄, filtered and concentrated under reduced pressure to give a residue, which was purified by flash silica gel chromatography (ISCO®; 40 g SepaFlash® silica flash column, eluent of 0~50~100% EA/PE gradient; flow 80 ml/min) to yield 1.2 g of the title compound as a mixture of diastereomers. The mixture was separated by preparative SFC (column: Daicel ChiralPak IG (250 mm×30 mm, 10 μm); mobile phase: A: CO₂, B: IPA (0.1% NH₃ in H₂O); B: 30%, isocratic elution mode) to yield 470 mg of peak 1 and 430 mg of peak 2. Analytical SFC data (Chiralpak IG-3 (50 mm×4.6 mm, 3 μm); A: CO₂, B: IPA (0.05% DEA)]; B: 5%-40%, flow 3 ml/min, T: 35° C.): P1: RT=1.76 min; P2: RT=1.95 min.

### Step 5: (2S)-2-Amino-N-(4-((R or S)-cyclopropyl((S)-2-oxo-4-(trifluoromethyl)imidazolidin-1-yl)methyl)pyridin-2-yl)-2-(4,4-difluorocyclohexyl)acetamide hydrochloride—Diastereomer 1

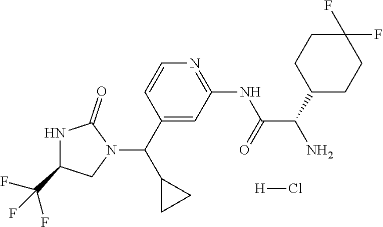

Tert-butyl ((1S)-2-((4-((R or S)-cyclopropyl((S)-2-oxo-4-(trifluoromethyl)imidazolidin-1-yl)methyl)pyridin-2-yl)amino)-1-(4,4-difluorocyclohexyl)-2-oxoethyl)carbamate (Step 4-P1, 230 mg) was dissolved in dioxane (2.5 ml) and HCl in dioxane solution (4 M, 2.5 ml) was added with stirring. After 5 h the mixture was concentrated in vacuo and the residue was lyophilised from ACN/water to yield 205 mg of the crude title compound. LC/MS: m/z=476.2 [M+H]⁺; rt: 1.33 min (LC/MS-method A).

### Step 5: (2S)-2-amino-N-(4-((R or S)-cyclopropyl((S)-2-oxo-4-(trifluoromethyl)imidazolidin-1-yl)methyl)pyridin-2-yl)-2-(4,4-difluorocyclohexyl)acetamide hydrochloride-diastereomer 2

Tert-butyl ((1S)-2-((4-((R or S)-cyclopropyl((S)-2-oxo-4-(trifluoromethyl)imidazolidin-1-yl)methyl)pyridin-2-yl)amino)-1-(4,4-difluorocyclohexyl)-2-oxoethyl)carbamate (Step 4-P2, 215 mg) was dissolved in dioxane (2.5 ml) and HCl in dioxane solution (4 M, 2.5 ml) was added with stirring. After 5 h the mixture was concentrated in vacuo and the residue was lyophilised from ACN/water to yield 210 mg of the crude title compound. LC/MS: m/z=476.2 [M+H]⁺; rt: 1.23 min (LC/MS-method A).

### Intermediate 49: (S)-2-Amino-2-(4,4-difluorocyclohexyl)-N-(4-((R)-1-(4-methyl-2-oxo-2,3-dihydro-1H-imidazol-1-yl)ethyl)pyridin-2-yl)acetamide

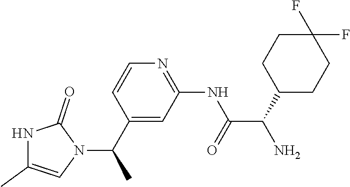 [SMILES: Cc1cn([C@H](C)c2ccnc(NC(=O)[C@@H](N)C3CCC(F)(F)CC3)c2)c(=O)[nH]1]

### Step 1: Tert-butyl (4-(1-((tert-butylsulfinyl)amino)ethyl)pyridin-2-yl)carbamate

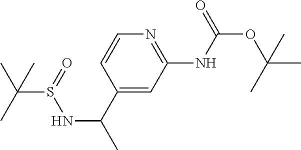 [SMILES: CC(NS(=O)C(C)(C)C)c1ccnc(NC(=O)OC(C)(C)C)c1]

To a solution of tert-butyl (Z)-(4-(((tert-butylsulfinyl)imino)methyl)pyridin-2-yl)carbamate (18 g) in THE (360 ml) was added dropwise MeMgBr (3 M in diethylether, 79.28 ml) at −78° C. under N₂. The mixture was warmed to 0° C. and stirred at 0° C. for 2 h.

Then the residue was poured into sat. aqueous NH₄Cl solution (250 ml) at 0° C. The aqueous phase was extracted with EA (300 ml, 2×). The combined organic phases were washed with brine (80 ml), dried with anhydrous Na₂SO₄, filtered and concentrated in vacuo to yield 19.88 g of the crude title compound.

### Step 2: 4-(1-Aminoethyl)pyridin-2-amine hydrochloride

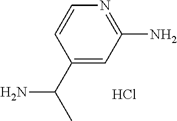

A mixture of tert-butyl (4-(1-((tert-butylsulfinyl)amino)ethyl)pyridin-2-yl)carbamate (18.31 g) in HCl/dioxane (4 M, 367 ml) was stirred at 25° C. for 12 h. Then the reaction mixture was concentrated under reduced pressure to yield 12.45 g of the crude title compound.

### Step-3A and -3B: Tert-butyl (S)-(1-(2-aminopyridin-4-yl)ethyl)carbamate (A) and tert-butyl (R)-(1-(2-aminopyridin-4-yl)ethyl)carbamate (B)

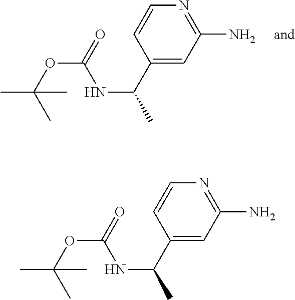 [SMILES: C[C@H](NC(=O)OC(C)(C)C)c1ccnc(N)c1 ; C[C@@H](NC(=O)OC(C)(C)C)c1ccnc(N)c1]

To a solution of 4-(1-aminoethyl)pyridin-2-amine hydrochloride (9.45 g) in MeOH (200 ml) was added a solution of Boc₂O (4.95 g) and TEA (12.34 ml) in MeOH (85 ml) dropwise. The mixture was stirred at −20° C. for 5 h. Then the reaction mixture was quenched by the addition HCl (1 M) at 25° C. to adjust the pH to 5-6 and concentrated under reduced pressure to give a residue which was purified by reversed-phase HPLC (column: Phenomenex luna C18, 250×80 mm; 10 μm; mobile phase: [A: water (FA 10%), B: ACN]; gradient: 5%-35% B over 20 min) to yield 5 g of a mixture of the racemic title compounds. The enantiomers were separated by SFC (column: REGIS (S,S) WHELK-O1 (250 mm×25 mm, 10 μm); mobile phase: [A: CO₂, B: MeOH (0.1% NH₃ in H₂O)]; B %: 30%, isocratic elution mode) to yield 1.66 g of title compound A and 1.58 g of title compound B.

### Step 4-BA and 4-BB: Benzyl ((S)-2-((4-((R)-1-((tert-butoxycarbonyl)amino)ethyl)pyridin-2-yl)amino)-1-(4,4-difluorocyclohexyl)-2-oxoethyl)carbamate (BA) and benzyl ((R)-2-((4-((R)-1-((tert-butoxycarbonyl)amino)ethyl)pyridin-2-yl)amino)-1-(4,4-difluorocyclohexyl)-2-oxoethyl)carbamate (BB)

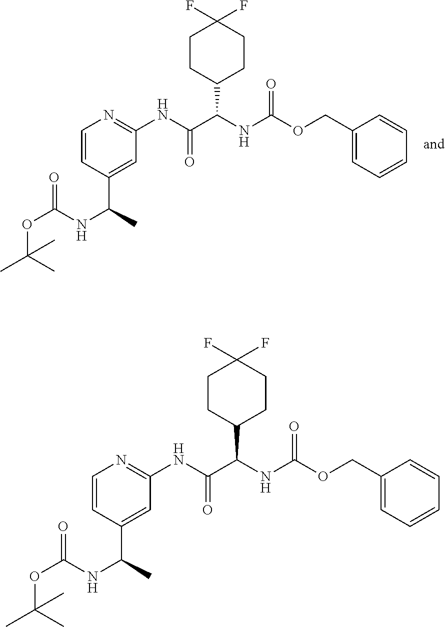 [SMILES: C[C@@H](NC(=O)OC(C)(C)C)c1ccnc(NC(=O)[C@@H](NC(=O)OCc2ccccc2)C2CCC(F)(F)CC2)c1 ; C[C@@H](NC(=O)OC(C)(C)C)c1ccnc(NC(=O)[C@H](NC(=O)OCc2ccccc2)C2CCC(F)(F)CC2)c1]

To a solution of 2-(((benzyloxy)carbonyl)amino)-2-(4,4-difluorocyclohexyl)acetic acid (5.62 g) in DCM (17 ml) was added DIEA (3.59 ml), EDCl (5.53 g), DMAP (5.04 g) and tert-butyl (R)-(1-(2-aminopyridin-4-yl)ethyl)carbamate (Int. 49-3B, 1.63 g). The mixture was stirred at 20° C. for 12 h. Then H₂O (30 ml) was added at 25° C., and the aqueous mixture was extracted with EA (30 ml, 3×). The combined organic layers were washed with 1 N HCl (50 ml, 2×), dried over Na₂SO₄, filtered and concentrated under reduced pressure to give a residue which was purified by column chromatography (SiO₂, PE/EA=1/0 to 1/1) to yield 3.88 g of a diasteromeric mixture of the title compounds.

The diastereomeric mixture was separated by SFC (column: Daicel Chiralpak-AD (250 mm×30 mm, 10 μm); mobile phase: [A: CO₂; B: IPA/ACN=4/1]; B %: 65%, isocratic elution mode) to yield 1.26 g of title compound BA and 1.24 g of title compound BB. Analytical SFC data (Chiralpak IG-3 (50 mm*4.6 mm, 3 μm); A: C₀₂, B: IPA (0.05% DEA)]; B %: 5%-40%, flow 3 ml/min, T: 35° C.). Step 4-BA: rt=0.64 min; Step 4-BB: rt=1.18 min

### Step 5: Benzyl ((S)-2-((4-((R)-1-aminoethyl)pyridin-2-yl)amino)-1-(4,4-difluorocyclohexyl)-2-oxoethyl)carbamate

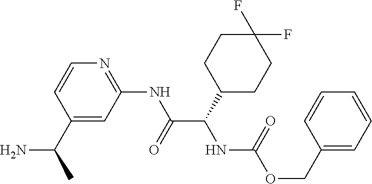 [SMILES: C[C@@H](N)c1ccnc(NC(=O)[C@@H](NC(=O)OCc2ccccc2)C2CCC(F)(F)CC2)c1]

Benzyl ((S)-2-((4-((R)-1-((tert-butoxycarbonyl)amino)ethyl)pyridin-2-yl)amino)-1-(4,4-difluorocyclohexyl)-2-oxoethyl)carbamate (Step 4-BA, 200 mg) was dissolved in dioxane (1.8 ml) and HCl in dioxane solution (4 M, 1.8 ml) was added with stirring. After stirring for 2.5 h the mixture was left standing overnight and then concentrated in vacuo. DCM and water were added to the residue and a neutral pH was established with sat. sodium bicarbonate solution. The organic phase was separated and the aqueous phase was extracted with DCM (2×). The combined organic layers were dried over Na₂SO₄, filtered and concentrated in vacuo to yield 177 mg of the crude title compound. LC/MS: m/z=447.2 [M+H]⁺; rt: 1.50 min (LC/MS-method A).

### Step A1: 2,5-Dioxopyrrolidin-1-yl (1,1-dimethoxypropan-2-yl)carbamate

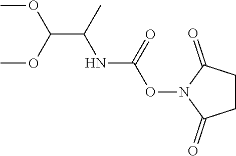 [SMILES: COC(OC)C(C)NC(=O)ON1C(=O)CCC1=O]

Pyridine (190 μl) and (2,5-dioxopyrrolidin-1-yl) carbonate (1.178 g) were mixed in ACN (5.5 ml). (1,1-dimethoxypropan-2-amine (240 mg) dissolved in ACN (2 ml) was added with stirring. After 30 min sat. NaHCO₃ solution and brine were added and the aqueous mixture was extracted with EE (2×). The combined organic phases were washed with brine, dried over Na₂SO₄, filtered and concentrated in vacuo. The residue was purified by column chromatography (12 g silica gel, nHep/EE=1/0 to 0/1) to yield 548 mg of the title compound which contained 1-hydroxypyrrolidine-2,5-dione as an impurity.

### Step 6: Benzyl ((1S)-1-(4,4-difluorocyclohexyl)-2-((4-((1R)-1-(3-(1,1-dimethoxypropan-2-yl)ureido)ethyl)pyridin-2-yl)amino)-2-oxoethyl)carbamate

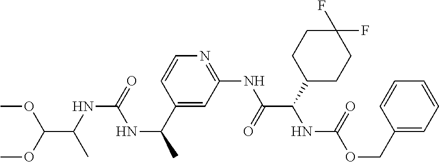 [SMILES: COC(OC)C(C)NC(=O)N[C@H](C)c1ccnc(NC(=O)[C@@H](NC(=O)OCc2ccccc2)C2CCC(F)(F)CC2)c1]

Benzyl ((S)-2-((4-((R)-1-aminoethyl)pyridin-2-yl)amino)-1-(4,4-difluorocyclohexyl)-2-oxoethyl)carbamate (Step 5, 175 mg) was dissolved in dry ACN. 2,5-Dioxopyrrolidin-1-yl (1,1-dimethoxypropan-2-yl)carbamate (Step A1, 247 mg) dissolved in ACN (3 ml) and TEA (273 μl) were added with stirring. After 1 h EE was added and the organic phase was washed with water (2×) and brine, dried over Na₂SO₄, filtered and concentrated in vacuo. The residue was purified by column chromatography (12 g silica gel, nHep/EE=1/0 to 0/1; 20 min) to yield 210 mg of the title compound. LC/MS: m/z=592.3 [M+H]⁺; rt: 2.05 min (LC/MS-method A).

### Step 7: Benzyl ((S)-1-(4,4-difluorocyclohexyl)-2-((4-((R)-1-(4-methyl-2-oxo-2,3-dihydro-1H-imidazol-1-yl)ethyl)pyridin-2-yl)amino)-2-oxoethyl)carbamate

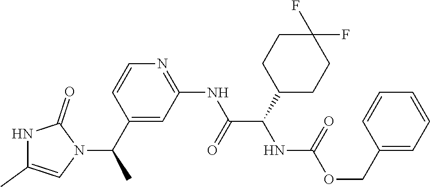 [SMILES: Cc1cn([C@H](C)c2ccnc(NC(=O)[C@@H](NC(=O)OCc3ccccc3)C3CCC(F)(F)CC3)c2)c(=O)[nH]1]

Benzyl ((1S)-1-(4,4-difluorocyclohexyl)-2-((4-((1R)-1-(3-(1,1-dimethoxypropan-2-yl)ureido)ethyl)pyridin-2-yl)amino)-2-oxoethyl)carbamate (209 mg) was dissolved in a solution of ACN/H₂O/TFA (4 ml; 5/1/1.5 ml) and stirred at 60° C. in a microwave oven for 2 min. Then sat. NaHCO₃ solution was added and the aqueous phase was extracted with EE (2×). The residue was purified by preparative RP HPLC (flow: 50 ml/min; gradient: 90% H₂O/10% ACN in 12.5 min to 10% H₂O/90% ACN; column: Agilent Prep C18 (10 μm, 30×250 mm). The pure product containing fractions were combined, the ACN partly removed and the remaining solution was lyophilised overnight to yield 119 mg of the title compound. LC/MS: m/z=528.2 [M+H]⁺; rt: 2.01 min (LC/MS-method A).

### Step 8: (S)-2-Amino-2-(4,4-difluorocyclohexyl)-N-(4-((R)-1-(4-methyl-2-oxo-2,3-dihydro-1H-imidazol-1-yl)ethyl)pyridin-2-yl)acetamide

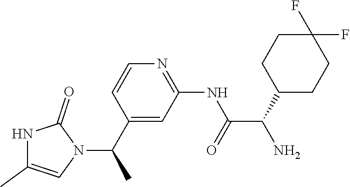 [SMILES: Cc1cn([C@H](C)c2ccnc(NC(=O)[C@@H](N)C3CCC(F)(F)CC3)c2)c(=O)[nH]1]

Benzyl ((S)-1-(4,4-difluorocyclohexyl)-2-((4-((R)-1-(4-methyl-2-oxo-2,3-dihydro-1H-imidazol-1-yl)ethyl)pyridin-2-yl)amino)-2-oxoethyl)carbamate (49 mg) was dissolved in MeOH (3 ml) under Ar atmosphere. Pd/C (5 mg, 10%) was added and the flask was purged with H₂ from a balloon. After stirring for 1 h under a H₂ atmosphere the catalyst was filtered off and washed with MeOH. The filtrate was concentrated and lyophilised from ACN/water overnight to yield 37 mg of the crude title compound. LC/MS: m/z=394.2 [M+H]⁺; rt: 1.09 min (LC/MS-method A).

### Intermediate 50: (2S)-2-Amino-2-(4,4-difluorocyclohexyl)-N-(4-(1-((S)-2-oxo-4-(trifluoromethyl)imidazolidin-1-yl)ethyl)pyridin-2-yl)acetamide hydrochloride

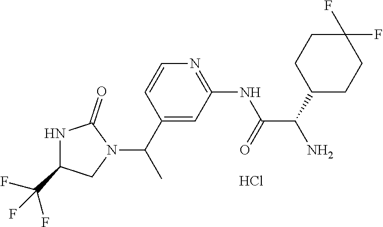 [SMILES: CC(c1ccnc(NC(=O)[C@@H](N)C2CCC(F)(F)CC2)c1)N1C[C@@H](C(F)(F)F)NC1=O ; Cl]

### Step 1: Tert-butyl (S)-(2-((4-bromopyridin-2-yl)amino)-1-(4,4-difluorocyclohexyl)-2-oxoethyl)carbamate

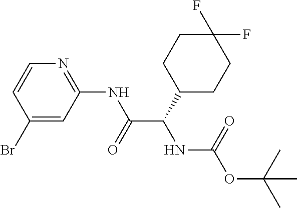 [SMILES: CC(C)(C)OC(=O)N[C@H](C(=O)Nc1cc(Br)ccn1)C1CCC(F)(F)CC1]

To a solution of 2-((tert-butoxycarbonyl)amino)-2-(4,4-difluorocyclohexyl)acetic acid (3.5 g) and 4-bromopyridin-2-amine (3.10 g) in pyridine (35 ml) was added T₃P (10.7 ml) at −20° C. The mixture was stirred at 25° C. for 12 h. Then the solvent was removed in vacuo and H₂O (250 ml) and EA (150 ml) were added. After separating the organic phase the aqueous phase was extracted with EA (150 ml). The combined organic layers were washed with NH₄Cl (100 ml), dried over anhydrous Na₂SO₄, filtered and concentrated under reduced pressure to give a residue, which was purified by column chromatography (SiO₂, PE/EA=1/0 to 4/1) to yield 2.53 g of the racemic title compound.

The racemate (5.6 g) was separated by SFC (column: Daicel Chiralcel OJ (250 mm*50 mm, 10 μm); mobile phase: B: [0.1% NH₃/H₂O in MeOH]; B %: 20%-20%; 20 min) to yield 2.5 g of the title compound (peak 1). Analytical SFC data (Chiralcel OJ-3 (50 mm×4.6 mm, 3 μm); A: CO₂, B: MeOH (0.05% DEA)]; B %: 5%-40%, flow 3 ml/min, T: 35° C.): peak 1: RT=0.58 min; peak 2: RT=0.76 min.

### Step 2: Tert-butyl (S)-(1-(4,4-difluorocyclohexyl)-2-oxo-2-((4-(prop-1-en-2-yl)pyridin-2-yl)amino)ethyl)carbamate

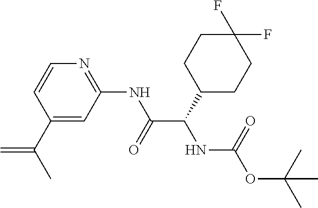 [SMILES: C=C(C)c1ccnc(NC(=O)[C@@H](NC(=O)OC(C)(C)C)C2CCC(F)(F)CC2)c1]

To tert-butyl (S)-(2-((4-bromopyridin-2-yl)amino)-1-(4,4-difluorocyclohexyl)-2-oxoethyl)carbamate (400 mg), 4,4,5,5-tetramethyl-2-(prop-1-en-2-yl)-1,3,2-dioxaborolane (225 mg), K₃PO₄ (568 mg) and Pd(dtbpf)Cl₂ (116 mg) were added dioxane (14 ml) and water (3.5 ml) under Ar. After flushing with Ar for 10 min the mixture was stirred at 100° C. for 45 min. The cooled mixture was poured into brine and the aqueous phase was extracted with EA (2×). The combined organic phases were dried over Na₂SO₄, filtered and concentrated in vacuo. The residue was purified by column chromatography (24 g silica gel, nHep/EA; 5 min 100% nHep, 100% nHep to 50% nHep in 40 min, 10 min at 50% nHep) to yield 345 mg of the title compound. LC/MS: m/z=410.1 [M+H]⁺; rt: 2.42 min (LC/MS-method A).

### Step 3: Tert-butyl (S)-(2-((4-acetylpyridin-2-yl)amino)-1-(4,4-difluorocyclohexyl)-2-oxoethyl)carbamate

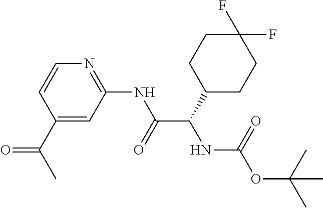 [SMILES: CC(=O)c1ccnc(NC(=O)[C@@H](NC(=O)OC(C)(C)C)C2CCC(F)(F)CC2)c1]

Tert-butyl (S)-(1-(4,4-difluorocyclohexyl)-2-oxo-2-((4-(prop-1-en-2-yl)pyridin-2-yl)amino)ethyl)carbamate (344 mg) was dissolved in dioxane (21 ml) and sodium periodate (898 mg) dissolved in water (21 ml) was added. After adding potassium osmate (IV) hydrate (56 mg) the mixture was stirred for 3 h. Then the mixture was poured into water and the aqueous phase was extracted with EA (2×). The combined organic phases were dried over Na₂SO₄, filtered and concentrated in vacuo. The residue was purified by column chromatography (12 g silica gel, nHep/EA; 5 min 100% nHep, 100% nHep to 50% nHep in 30 min, 10 min at 50% nHep) to yield 318 mg of the title compound. LC/MS: m/z=412.2 [M+H]⁺; rt: 2.14 min (LC/MS-method A).

### Step 4: Tert-butyl ((1S)-2-((4-(1-(((S)-2-amino-3,3,3-trifluoropropyl)amino)ethyl)pyridin-2-yl)amino)-1-(4,4-difluorocyclohexyl)-2-oxoethyl)carbamate

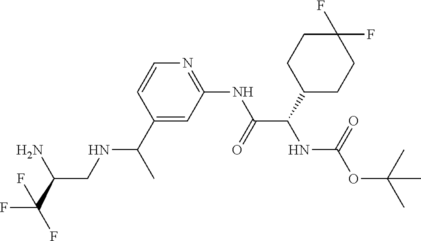 [SMILES: CC(NC[C@H](N)C(F)(F)F)c1ccnc(NC(=O)[C@@H](NC(=O)OC(C)(C)C)C2CCC(F)(F)CC2)c1]

Tert-butyl (S)-(2-((4-acetylpyridin-2-yl)amino)-1-(4,4-difluorocyclohexyl)-2-oxoethyl)carbamate (220 mg) and (S)-3,3,3-trifluoropropane-1,2-diamine dihydrochloride (Int. 1, Step B6; 129 mg) were dissolved in DCM (10 ml) under Ar. TEA (328 μl) was added and after stirring for 10 min at RT the mixture was stirred for 1 h at 40° C. After cooling to RT sodium cyanoborohydride (118 mg), dry methanol (6 ml) and acetic acid (0.159 ml) were added and stirring was continued for ~70 h. The mixture was poured into saturated NaHCO₃ solution and DCM was added. The organic layer was separated and the aqueous phase was extracted with DCM (2×). The combined organic phases were dried over sodium sulphate, filtered and concentrated in vacuo. The residue was further purified by preparative RP HPLC (flow: 25 ml/min; gradient: 100% H₂O+0.05% TFA for 20 min, in 30 min from 100% H₂O+0.05% TFA to 55% H₂O+0.05% TFA/45% ACN; column: Purosphere® STAR-RP18, 25 mm×250 mm, 10 μm). The pure product containing fractions were combined, the ACN was partly removed and the TFA neutralised with sat. NaHCO₃ solution. After extracting the product with DCM (3×) the combined organic phases were dried over a ChemElut cartridge and concentrated in vacuo to yield 192 mg of the title compound. LC/MS: m/z=524.2 [M+H]⁺; rt: 1.52 min (LC/MS-method A).

### Step 5-P1 and -P2: tert-butyl ((S)-1-(4,4-difluorocyclohexyl)-2-oxo-2-((4-((R or S)-1-((S)-2-oxo-4-(trifluoromethyl)imidazolidin-1-yl)ethyl)pyridin-2-yl)amino)ethyl)carbamate

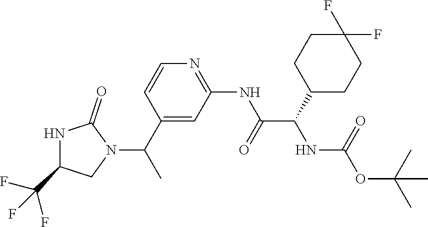 [SMILES: CC(c1ccnc(NC(=O)[C@@H](NC(=O)OC(C)(C)C)C2CCC(F)(F)CC2)c1)N1C[C@@H](C(F)(F)F)NC1=O]

Tert-butyl ((1S)-2-((4-(1-(((S)-2-amino-3,3,3-trifluoropropyl)amino)ethyl)pyridin-2-yl)amino)-1-(4,4-difluorocyclohexyl)-2-oxoethyl)carbamate (191 mg) was dissolved in dry THE (5.5 ml) and the mixture was heated to 65° C. for 5 min. Then CDI (177 mg) was added and stirring was continued for 6 h. Then the mixture was poured into sat. NaHCO₃ solution and the aqueous phase was extracted with DCM (2×). The combined organic phases were dried over Na₂SO₄, filtered and concentrated in vacuo. The residue was purified by preparative RP HPLC (flow: 15 ml/min; gradient: 95% H₂O+0.05% TFA/5% ACN in 45 min to 10% H₂O+0.05% TFA/90% ACN; column: XBridge BEH130, Prep C18, 10 μM, OBD (19×250 mm)). The pure fractions of peak 2 were combined, the ACN was removed, pH adjusted to ~7 and the aqueous phase was extracted with DCM (2×). The combined organic phases were dried over Na₂SO₄, filtered and concentrated in vacuo. The residue was lyophilised from ACN/H₂O to yield 74 mg of P2. The fractions of peak 1 were combined, the ACN was reduced in vacuo and the residue lyophilised. RP HPLC was repeated with the conditions described above. The pure fractions of P1 were treated as described for P2 to yield 41 mg of P1 after lyophilisation. DS1: LC/MS: m/z=550.2 [M+H]⁺; rt: 2.08 min (LC/MS-method A). DS2: LC/MS: m/z=550.2 [M+H]⁺; rt: 2.17 min (LC/MS-method A).

### Step 6: (S)-2-Amino-2-(4,4-difluorocyclohexyl)-N-(4-((R or S)-1-((S)-2-oxo-4-(trifluoromethyl)imidazolidin-1-yl)ethyl)pyridin-2-yl)acetamide hydrochloride DS1

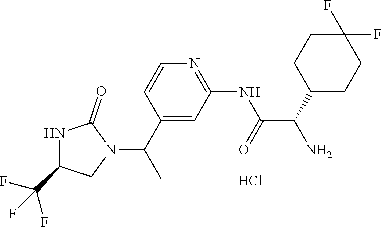 [SMILES: CC(c1ccnc(NC(=O)[C@@H](N)C2CCC(F)(F)CC2)c1)N1C[C@@H](C(F)(F)F)NC1=O ; Cl]

Tert-butyl ((1S)-1-(4,4-difluorocyclohexyl)-2-oxo-2-((4-(1-((S)-2-oxo-4-(trifluoromethyl)imidazolidin-1-yl)ethyl)pyridin-2-yl)amino)ethyl)carbamate (Step 5-P1, 41 mg) was dissolved in dry dioxane (1.5 ml) and HCl solution (1.5 ml; 4 M in dioxane) was added dropwise with stirring. After 2 h the mixture was concentrated in vacuo and the residue was lyophilized from ACN/H₂O to yield 42 mg of the crude title compound. LC/MS: m/z=450.3 [M+H]⁺; rt: 1.13 min (LC/MS-method A).

### Intermediate 51-1-DS1: Tert-butyl ((1S)-1-(4,4-difluorocyclohexyl)-2-((4-((R or S)-2-methoxy-1-((3S,5S)-2-oxo-5-(trifluoromethyl)pyrrolidin-3-yl)ethyl)pyridin-2-yl)amino)-2-oxoethyl)carbamate

### Intermediate 51-2-DS1: Tert-butyl ((1S)-1-(4,4-difluorocyclohexyl)-2-((4-((R or S)-2-methoxy-1-((3R,5S)-2-oxo-5-(trifluoromethyl)pyrrolidin-3-yl)ethyl)pyridin-2-yl)amino)-2-oxoethyl)carbamate

### Intermediate 51-2-DS2: Tert-butyl ((1S)-1-(4,4-difluorocyclohexyl)-2-((4-((R or S)-2-methoxy-1-((3R,5S)-2-oxo-5-(trifluoromethyl)pyrrolidin-3-yl)ethyl)pyridin-2-yl)amino)-2-oxoethyl)carbamate

### Step A1: 2-(3-Methoxyprop-1-en-2-yl)-4,4,5,5-tetramethyl-1,3,2-dioxaborolane

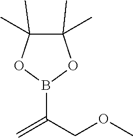

To a solution of 3-methoxyprop-1-yne (3.6 g, 4.24 ml), CuCl (508 mg), 4,4,4′,4′,5,5,5′,5′-octamethyl-2,2′-bi(1,3,2-dioxaborolane) (13.04 g), tri-tert-butylphosphonium tetrafluoroborate (1.79 g) and t-BuONa (740 mg) in toluene (70 ml) was dropwise added MeOH (4.16 ml) under N₂ at 0° C. over 30 min. The mixture was stirred at 25° C. for 2 h. The reaction mixture was quenched with MeOH (20 ml) at 0° C. and filtered. The filtrate was concentrated in vacuo to give a residue, which was purified by column chromatography (SiO₂, PE:EA=100:1 to 80:1) to yield 5.8 g of the title compound. ¹H NMR (400 MHz, CDCl₃) δ ppm: 5.93 (br, 1H), 5.90 (br, 1H), 4.03 (s, 2H), 3.36 (s, 3H), 1.27 (s, 12H).

### Step A2: 2-Chloro-4-(3-methoxyprop-1-en-2-yl)pyridine

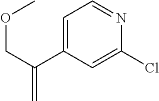

A mixture of 4-bromo-2-chloropyridine (38 g), 2-(3-methoxyprop-1-en-2-yl)-4,4,5,5-tetramethyl-1,3,2-dioxaborolane (43 g), Ruphos Pd G₃ (1.65 g) and K₃PO₄ (126 g) in dioxane (400 ml) and water (50 ml) was stirred at 100° C. for 2 h under N₂. Then brine (200 ml) was added and the aqueous phase was extracted with EA (200 ml). The organic layer was dried over Na₂SO₄, filtered and concentrated in vacuo to give a residue, which was purified by column chromatography (SiO₂, PE/EA=3/1) to yield 21 g of the title compound. LC/MS: m/z=184.2 [M+H]⁺; rt: 0.36 min (LC/MS-method C).

### Step A3: 1-(2-Chloropyridin-4-yl)-2-methoxyethan-1-one

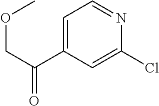

A mixture of 2-chloro-4-(3-methoxyprop-1-en-2-yl)pyridine (21 g), NalO₄ (2.45 g), K₂OsO₄ (126.4 g) in dioxane (200 ml) and water (40 ml) was stirred at 25° C. for 2 h under N₂. Then sat. NaHCO₃ solution (100 ml) was added and the aqueous phase was extracted with EA (100 ml). The organic layer was washed with brine (30 ml), dried over Na₂SO₄, filtered and concentrated in vacuo to give a residue, which was purified by column chromatography (SiO₂, PE/EA=5/1 to 3/1) to yield 10 g of the title compound. LC/MS: m/z=186.2 [M+H]⁺; rt: 0.27 min (LC/MS-method C).

### Step A4: 1-(2-Chloropyridin-4-yl)-2-methoxyethan-1-ol

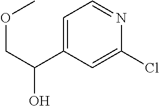

To a solution of 1-(2-chloropyridin-4-yl)-2-methoxyethan-1-one (1 g) in THE (10 ml) was added NaBH₄ (204 mg) at 0° C. with stirring. After stirring at 0° C. for 1 h the reaction mixture was quenched with sat. aqueous NH₄Cl solution (40 ml) and the aqueous phase was extracted with EA (40 ml, 2×). The combined organic layers were dried over Na₂SO₄, filtered and concentrated in vacuo to give a residue, which was purified by column chromatography (SiO₂, PE/EA=5/1 to 2/1) to yield 0.7 g of the title compound. LC/MS: m/z=188.3 [M+H]⁺; rt: 0.22 min (LC/MS-method C).

### Step A5: 4-(1-Bromo-2-methoxyethyl)-2-chloropyridine

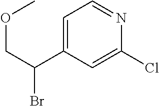

To a mixture of 1-(2-chloropyridin-4-yl)-2-methoxyethan-1-ol (7.15 g) and PPh3 (19.99 g) in DCM (60 ml) was added TEA (6.37 ml) followed by CBr₄ (25.28 g) at 0° C. with stirring. After stirring at 20° C. for 16 h the mixture was concentrated and the residue was purified by column chromatography (SiO2, PE=5/1 to 3/1) to yield 4.9 g of the title compound. LC/MS: m/z=250.1 [M+H]⁺; rt: 0.38 min (LC/MS-method C).

### Step B1: Dimethyl 2-(2,2-dimethoxyethyl)malonate

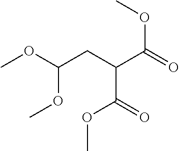

To a solution of dimethyl malonate (50 g) in DMF (600 ml) was added NaH (15.3 g, 60%) at 0° C. and the mixture was stirred for 30 min. Then KI (11.3 g) and 2-bromo-1,1-dimethoxyethane (64.17 g) were added with stirring. The mixture was allowed to warm to 25° C., then heated to 100° C. and kept at this temperature for 12 h. Then the mixture was diluted with water (1.5 l) at 0° C. and the aqueous phase was extracted with EA (800 ml, 3×). The combined organic layers were washed with brine (1 l, 2×), dried over anhydrous Na₂SO₄, filtered and concentrated under reduced pressure to give a residue, which was purified by column chromatography (SiO₂, PE/EA=1/0 to 4/1) to yield 41.62 g of the title compound. ¹H NMR (400 MHz, CDCl₃) δ ppm: 4.42 (t, J=5.4 Hz, 1H), 3.75 (s, 6H), 3.54 (t, J=7.2 Hz, 1H), 3.33 (s, 6H), 2.24 (dd, J=5.6, 7.2 Hz, 2H).

### Step B2: Dimethyl 2-(2-oxoethyl)malonate

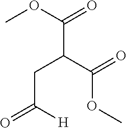

To a solution of dimethyl 2-(2,2-dimethoxyethyl)malonate (41.6 g) in DCM (250 ml) and H₂O (250 ml) was added TFA (140 ml) at 0° C. The mixture was stirred at 25° C. for 1.5 h. Then the reaction mixture was quenched with saturated K₂CO₃ solution to pH 6~8 at 0° C. and the aqueous phase was extracted with DCM (350 ml, 3×). The combined organic layers were washed with brine (600 ml, 2×), dried over anhydrous Na₂SO₄, filtered and concentrated under reduced pressure to yield 33 g of the crude title compound.

### Step B3: Dimethyl (R,E)-2-(2-((tert-butylsulfinyl)imino)ethyl)malonate

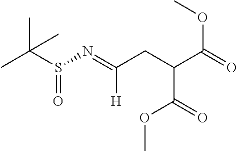 [SMILES: COC(=O)C(C/C=N/[S@](=O)C(C)(C)C)C(=O)OC]

To a solution of dimethyl 2-(2-oxoethyl)malonate (33 g) and (R)-2-methylpropane-2-sulfinamide (29.86 g) in DCM (350 ml) was added pyrrolidine (1.58 ml) and the mixture was stirred at 25° C. for 12 h. Then the reaction mixture was concentrated under reduced pressure to give a residue, which was purified by column chromatography (SiO₂, PE/EA=I/O to 3/2) to yield 27.73 g of the title compound. LC/MS: m/z=278.2 [M+H]⁺; rt: 0.29 min (LC/MS-method C).

### Step B4: Dimethyl 2-((S)-2-(((R)-tert-butylsulfinyl)amino)-3,3,3-trifluoropropyl)malonate

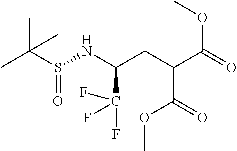 [SMILES: COC(=O)C(C[C@H](N[S@](=O)C(C)(C)C)C(F)(F)F)C(=O)OC]

To a solution of dimethyl (R,E)-2-(2-((tert-butylsulfinyl)imino)ethyl)malonate (27.7 g) and tetrabutylammonium acetate (36.14 g) in THE (554 ml) was added TMS-CF₃ (35.51 g) at −55° C. with stirring. After stirring at −55° C. for 1 h the mixture was stirred at 0° C. for 3 h. Then the mixture was quenched with aqueous ammonium chloride solution (100 ml). The mixture was diluted with water (1.2 l) and extracted with EA (500 ml, 3×). The combined organic layers were washed with brine (500 ml, 3×), dried over anhydrous Na₂SO₄ and concentrated in vacuo to give a residue, which was purified by column chromatography (SiO₂, PE=1/0 to 3/2) to yield 12 g of the title compound. LC/MS: m/z=348.1 [M+H]⁺; rt: 0.35 min (LC/MS-method C).

### Step 1: Methyl (5S)-3-(1-(2-chloropyridin-4-yl)-2-methoxyethyl)-2-oxo-5-(trifluoromethyl)pyrrolidine-3-carboxylate

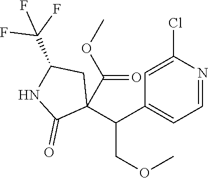 [SMILES: COCC(c1ccnc(Cl)c1)C1(C(=O)OC)C[C@@H](C(F)(F)F)NC1=O]

A mixture of 4-(1-bromo-2-methoxyethyl)-2-chloropyridine (Step A5; 4.4 g), dimethyl 2-((S)-2-(((R)-tert-butylsulfinyl)amino)-3,3,3-trifluoropropyl)malonate (Step B4; 6.10 g) and K₂CO₃ (4.85 g) in DMF (80 ml) was stirred at 25° C. for 1 h and then 60° C. for 1 h. Then brine was added (50 ml) and the aqueous phase was extracted with EA (50 ml). The organic layer was washed with brine (20 ml, 2×), dried over Na₂SO₄, filtered and concentrated in vacuo to give a residue, which was purified by column chromatography (SiO₂, PE/EA=5/1 to 1/1) to yield 402 mg of the title compound. LC/MS: m/z=381.0 [M+H]⁺; rt: 0.58 min (LC/MS-method C).

### Step 2-P1 and -P2: (5S)-3-(1-(2-Chloropyridin-4-yl)-2-methoxyethyl)-5-(trifluoromethyl)pyrrolidin-2-one

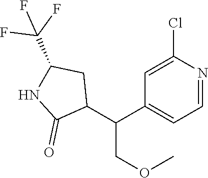 [SMILES: COCC(c1ccnc(Cl)c1)C1C[C@@H](C(F)(F)F)NC1=O]

To a solution of methyl (5S)-3-(1-(2-chloropyridin-4-yl)-2-methoxyethyl)-2-oxo-5-(trifluoromethyl)pyrrolidine-3-carboxylate (402 mg) in THE (10 ml) and water (1 ml) was added LiOH·H₂O (89 mg). After stirring at 25° C. for 1 h the pH was adjusted to 6 with HCl (3 M) and the aqueous phase was extracted with EA (5 ml, 2×). The combined organic layers were dried over Na₂SO₄, filtered and concentrated in vacuo to give a residue. The residue was diluted with toluene (3 ml) and then stirred at 80° C. for 1 h. Then the solution was concentrated in vacuo to give a residue, which was purified by column chromatography (SiO₂, PE/EA=3/1 to 1/1) to yield 70 mg of P1 and 288 mg of P2 of the title compound. P1: LC/MS: m/z=323.0 [M+H]⁺; rt: 0.35 min (LC/MS-method C). P2: LC/MS: m/z=323.0 [M+H]⁺; rt: 0.35 min (LC/MS-method C).

### Step 3-P1: Tert-butyl (4-(2-methoxy-1-((5S)-2-oxo-5-(trifluoromethyl)pyrrolidin-3-yl)ethyl)pyridin-2-yl)carbamate (diastereomer A)

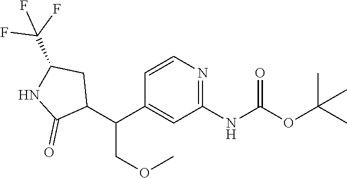 [SMILES: COCC(c1ccnc(NC(=O)OC(C)(C)C)c1)C1C[C@@H](C(F)(F)F)NC1=O]

To a solution of (5S)-3-(1-(2-chloropyridin-4-yl)-2-methoxyethyl)-5-(trifluoromethyl)pyrrolidin-2-one (Step 2-P1, 70 mg) and tert-butyl carbamate (38.12 mg) in dioxane (2 ml) was added Pd(OAc)₂ (9.7 mg), Cs₂CO₃ (141 mg) and XPhos (31 mg). After stirring at 100° C. under N₂ for 1 h the reaction mixture was filtered. Brine (2 ml) was added to the filtrate and the aqueous phase was extracted with EA (2 ml, 2×). The combined organic layers were dried over Na₂SO₄, filtered and concentrated in vacuo to give a residue, which was purified by column chromatography (SiO₂, PE/EA=2/1 to 1/1) to yield 44 mg of the title compound. LC/MS: m/z=404.0 [M+H]⁺; rt: 0.32 min (LC/MS-method C). Analytical SFC data (Chirapak AD-3 (50 mm×4.6 mm, 3 μm); A: CO₂, B: MeOH (0.05% DEA)]; B %: 5%-40%, flow 3 ml/min, T: 35° C.): rt=0.68 min.

### Step 4-P1: (5S)-3-(1-(2-Aminopyridin-4-yl)-2-methoxyethyl)-5-(trifluoromethyl)pyrrolidin-2-one

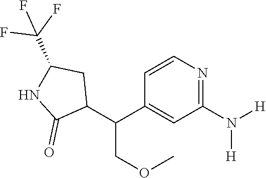 [SMILES: COCC(c1ccnc(N)c1)C1C[C@@H](C(F)(F)F)NC1=O]

To a solution of tert-butyl (4-(2-methoxy-1-((5S)-2-oxo-5-(trifluoromethyl)pyrrolidin-3-yl)ethyl)pyridin-2-yl)carbamate (Step 3-P1, 42 mg) in DCM (0.5 ml) was added TFA (0.25 ml). After stirring at 25° C. for 12 h the reaction mixture was concentrated in vacuo to give a residue which was adjusted with sat. NaHCO₃ solution to pH=8. The aqueous mixture was extracted with EA (2 ml, 3×) and the combined organic layers were dried over Na₂SO₄, filtered and concentrated in vacuo to yield 28 mg of the title compound. LC/MS: m/z=304.1 [M+H]⁺; rt: 0.21 min (LC/MS-method C).

### Step 5-P1: Tert-butyl ((1S)-1-(4,4-difluorocyclohexyl)-2-((4-((R or S)-2-methoxy-1-((3S,5S)-2-oxo-5-(trifluoromethyl)pyrrolidin-3-yl)ethyl)pyridin-2-yl)amino)-2-oxoethyl)carbamate

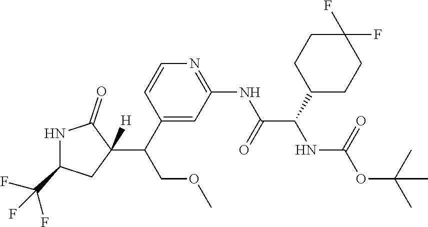 [SMILES: COCC(c1ccnc(NC(=O)[C@@H](NC(=O)OC(C)(C)C)C2CCC(F)(F)CC2)c1)[C@@H]1C[C@@H](C(F)(F)F)NC1=O]

A mixture of (5S)-3-(1-(2-aminopyridin-4-yl)-2-methoxyethyl)-5-(trifluoromethyl)pyrrolidin-2-one (27 mg), (S)-2-((tert-butoxycarbonyl)amino)-2-(4,4-difluorocyclohexyl)acetic acid (28 mg) and EDCl (35.4 mg) in pyridine (0.5 ml) was stirred at 25° C. for 12 h. Then brine (5 ml) was added and the aqueous phase was extracted with EA (5 ml). The organic layer was dried over Na₂SO₄, filtered and concentrated in vacuo to give a residue, which was purified by column chromatography (SiO₂, PE/EA=3/1 to 1/1) to yield 15 mg of the title compound. The stereochemistry was tentatively assigned. LC/MS: m/z=579.2 [M+H]⁺; rt: 0.44 min (LC/MS-method C). Analytical SFC data (Chiralcel OD-3 (50 mm×4.6 mm, 3 μm); A: CO₂, B: MeOH (0.05% DEA)]; B %: 5%-40%, flow 3 ml/min, T: 35° C.): rt=0.89 min.

### Step 3-P2A and -P2B: Tert-butyl (4-(2-methoxy-1-((5S)-2-oxo-5-(trifluoromethyl)pyrrolidin-3-yl)ethyl)pyridin-2-yl)carbamate

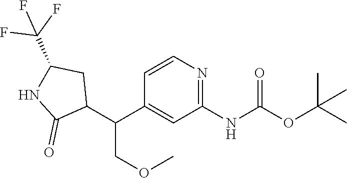 [SMILES: COCC(c1ccnc(NC(=O)OC(C)(C)C)c1)C1C[C@@H](C(F)(F)F)NC1=O]

Following the procedure described in Step 3-P1 using (5S)-3-(1-(2-chloropyridin-4-yl)-2-methoxyethyl)-5-(trifluoromethyl)pyrrolidin-2-one (Step 2-P2, 250 mg) 130 mg of a mixture of diastereomers was obtained as identified by analytical SFC in a ratio of about 2:1. LC/MS: m/z=404.1 [M+H]⁺; rt: 0.32 min (LC/MS-method C). Analytical SFC data (Chiralpak AD-3 (50 mm×4.6 mm, 3 μm); A: CO₂, B: MeOH (0.05% DEA)]; B %: 5%-40%, flow 3 ml/min, T: 35° C.): B: rt=0.80 min (67%), C: 1.00 min (33%).

### Step 5-P2A and -P2B: Tert-butyl ((1S)-1-(4,4-difluorocyclohexyl)-2-((4-((R or S)-2-methoxy-1-((3R,5S)-2-oxo-5-(trifluoromethyl)pyrrolidin-3-yl)ethyl)pyridin-2-yl)amino)-2-oxoethyl)carbamate (P2A) and tert-butyl ((1S)-1-(4,4-difluorocyclohexyl)-2-((4-((R or S)-2-methoxy-1-((3R,5S)-2-oxo-5-(trifluoromethyl)pyrrolidin-3-yl)ethyl)pyridin-2-yl)amino)-2-oxoethyl)carbamate (P2B)

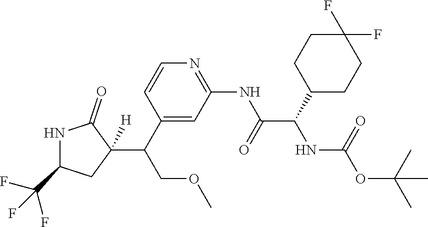 [SMILES: COCC(c1ccnc(NC(=O)[C@@H](NC(=O)OC(C)(C)C)C2CCC(F)(F)CC2)c1)[C@H]1C[C@@H](C(F)(F)F)NC1=O]

Treating tert-butyl (4-(2-methoxy-1-((5S)-2-oxo-5-(trifluoromethyl)pyrrolidin-3-yl)ethyl)pyridin-2-yl)carbamate (Step 3-P2A and -P2B, 130 mg) as described for Step 4-P1 and Step 5-P1 120 mg of the diastereomeric mixture of the title compounds was obtained.

This mixture (120 mg) was purified by SFC (column: Daicel Chiralpak AS (250 mm×30 mm, 10 μm), A: CO₂, B: EtOH (0.1% NH₃ in H₂O), B 20% isocratic mode, flow 100 ml/min) to yield 50 mg of I-051-5-P2A and 30 mg of I-051-5-P2B. The stereochemistry was tentatively assigned. Analytical SFC data (Chiralpak AS-3 (50 mm×4.6 mm, 3 μm); A: CO₂, B: EtOH (0.05% DEA)]; B %: 5%-40%, flow 3 ml/min, T: 35° C.): P2A: rt=1.09 min; P2B: 1.28 min.

### Step 6: Intermediate 51-1-DS1: (2S)-2-Amino-2-(4,4-difluorocyclohexyl)-N-(4-((R or S)-2-methoxy-1-((3S,5S)-2-oxo-5-(trifluoromethyl)pyrrolidin-3-yl)ethyl)pyridin-2-yl)acetamide DS1

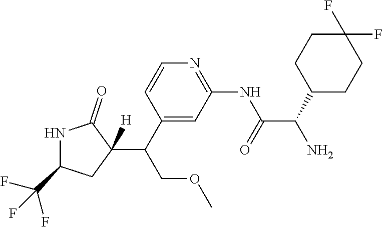 [SMILES: COCC(c1ccnc(NC(=O)[C@@H](N)C2CCC(F)(F)CC2)c1)[C@@H]1C[C@@H](C(F)(F)F)NC1=O]

Tert-butyl ((1S)-1-(4,4-difluorocyclohexyl)-2-((4-((R or S)-2-methoxy-1-((3S,5S)-2-oxo-5-(trifluoromethyl)pyrrolidin-3-yl)ethyl)pyridin-2-yl)amino)-2-oxoethyl)carbamate (Step 5-P1, 8 mg) was dissolved in dry dioxane (0.5 ml) and HCl solution (0.5 ml; 4 M in dioxane) was added dropwise with stirring. After stirring for 2.5 h the mixture stood overnight. Then DCM and water were added and the pH was adjusted to neutral with sat. NaHCO₃ solution. The organic phase was separated and the aqueous phase was extracted with DCM (3×). The combined organic phases were dried over Na₂SO₄, filtered and concentrated in vacuo. The residue was lyophilised from ACN/H₂O to yield 7 mg of the crude title compound. LC/MS: m/z=479.2 [M+H]⁺; rt: 1.22 min (LC/MS-method A).

### Step 6: Intermediate 51-2-DS1 (2S)-2-Amino-2-(4,4-difluorocyclohexyl)-N-(4-((R or S)-2-methoxy-1-((3R,5S)-2-oxo-5-(trifluoromethyl)pyrrolidin-3-yl)ethyl)pyridin-2-yl)acetamide DS1

Following the procedure for DS1 tert-butyl ((1S)-1-(4,4-difluorocyclohexyl)-2-((4-((R or S)-2-methoxy-1-((3R,5S)-2-oxo-5-(trifluoromethyl)pyrrolidin-3-yl)ethyl)pyridin-2-yl)amino)-2-oxoethyl)carbamate (Step 5-P2A, 30 mg) gave 25 mg of the crude title compound. LC/MS: m/z=479.2 [M+H]⁺; rt: 1.21 min (LC/MS-method A).

### Step 6: Intermediate 51-2-DS2: (2S)-2-Amino-2-(4,4-difluorocyclohexyl)-N-(4-((R or S)-2-methoxy-1-((3R,5S)-2-oxo-5-(trifluoromethyl)pyrrolidin-3-yl)ethyl) pyridin-2-yl)acetamide DS2

Following the procedure for Intermediate 51-1-DS1, tert-butyl ((1S)-1-(4,4-difluorocyclohexyl)-2-((4-((R or S)-2-methoxy-1-((3R,5S)-2-oxo-5-(trifluoromethyl)pyrrolidin-3-yl)ethyl)pyridin-2-yl)amino)-2-oxoethyl)carbamate (Step 5-P2B, 15 mg) gave 13 mg of the crude title compound. LC/MS: m/z=479.2 [M+H]⁺; rt: 1.16 min (LC/MS-method A).

### Intermediate 52: (S)-2-Amino-2-(4,4-difluorocyclohexyl)-N-(4-((S)-2-methoxy-1-((S)-2-oxo-4-(trifluoromethyl)imidazolidin-1-yl)ethyl)pyridin-2-yl)acetamide hydrochloride

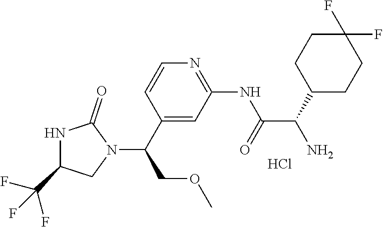 [SMILES: COC[C@H](c1ccnc(NC(=O)[C@@H](N)C2CCC(F)(F)CC2)c1)N1C[C@@H](C(F)(F)F)NC1=O ; Cl]

### Step 1: Tert-butyl ((S)-1-(4,4-difluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)carbamate

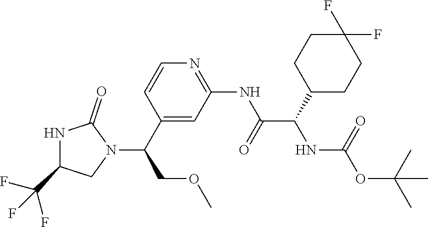 [SMILES: COC[C@H](c1ccnc(NC(=O)[C@@H](NC(=O)OC(C)(C)C)C2CCC(F)(F)CC2)c1)N1C[C@@H](C(F)(F)F)NC1=O]

To a solution of (S)-1-((S)-1-(2-Aminopyridin-4-yl)-2-methoxyethyl)-4-(trifluoromethyl)imidazolidin-2-one hydrochloride (Int. 54-B-P1, 6 g) in pyridine (60 ml) was added (S)-2-((tert-butoxycarbonyl)amino)-2-(4,4-difluorocyclohexyl)acetic acid (5.78 g) and EDCl (11.34 g). The mixture was stirred at 25° C. for 2 h. Then the reaction mixture was concentrated under reduced pressure to give a residue which was purified by prep-HPLC (column: Phenomenex luna 018 (250×70 mm, 10 μm); mobile phase: A: water (FA 10%), B ACN; gradient: 30%-60% B over 15 min) followed by SFC (column: Daicel Chiralpak AD (250 mm×30 mm, 10 μm); mobile phase: [A: 002, B: IPA (0.1% NH₃ in H₂O)]; B %: 50%, isocratic elution mode) to yield 2.93 g of the title compound. LC/MS: m/z=580.2 [M+H]⁺; rt: 0.44 min (LC/MS-method C). Analytical SFC data: (Chiralpak AD-3 (50×4.6 mm, 3 μm); A: 002, B: IPA (0.05% DEA)]; B %: 5%-40%, flow 3 ml/min, T: 35° C.): RT=1.57 min.

### Step 2: (S)-2-Amino-2-(4,4-difluorocyclohexyl)-N-(4-((S)-2-methoxy-1-((S)-2-oxo-4-(trifluoromethyl)imidazolidin-1-yl)ethyl)pyridin-2-yl)acetamide hydrochloride

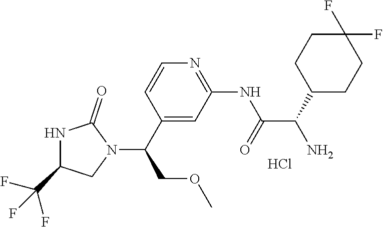 [SMILES: COC[C@H](c1ccnc(NC(=O)[C@@H](N)C2CCC(F)(F)CC2)c1)N1C[C@@H](C(F)(F)F)NC1=O ; Cl]

Tert-butyl ((S)-1-(4,4-difluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)carbamate (251 mg) was dissolved in dry dioxane (3.0 ml) and Hoi solution (3.0 ml; 4 M in dioxane) was added dropwise with stirring. After 2 h the mixture was concentrated in vacuo and the residue was lyophilized from ACN/H₂O to yield 240 mg of the crude title compound. LC/MS: m/z=480.3 [M+H]⁺; rt: 1.14 min (LC/MS-method A).

### Intermediate 53-P1 and P2: 1-(Ethyl-d5)-1H-pyrazole-5-carboxylic acid and 1-(ethyl-d5)-1H-pyrazole-3-carboxylic acid

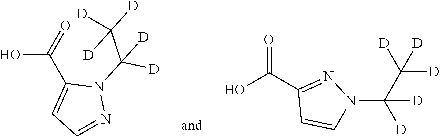 [SMILES: [2H]C([2H])([2H])C([2H])([2H])n1nccc1C(=O)O ; [2H]C([2H])([2H])C([2H])([2H])n1ccc(C(=O)O)n1]

### Step 1-P1 and P2: Ethyl 1-(ethyl-d5)-1H-pyrazole-5-carboxylate (P1) and ethyl 1-(ethyl-d5)-1H-pyrazole-3-carboxylate (P2)

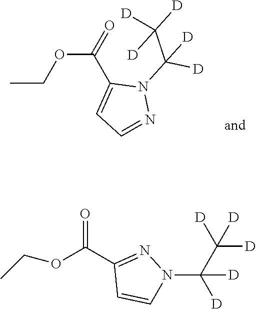 [SMILES: [2H]C([2H])([2H])C([2H])([2H])n1nccc1C(=O)OCC ; [2H]C([2H])([2H])C([2H])([2H])n1ccc(C(=O)OCC)n1]

DMF (3.1 ml) was added to ethyl 1H-pyrazole-5-carboxylate (510 mg) and potassium carbonate (754 mg). With stirring 1-bromoethane-1,1,2,2,2-d5 (415 mg) was added and stirring was continued overnight. Then the mixture was poured into water and the aqueous phase was extracted with EA (2×). The combined organic phases were washed with brine, dried over Na₂SO₄, filtered and concentrated in vacuo. The residue was purified by silica gel chromatography (nHep/EA: 90:10 to 40:60 in 30 min). The pure the respective compound containing fractions were combined and concentrated in vacuo to yield 193 mg of P1 and 267 mg of P2.

P1: LC/MS: m/z=174.0 [M+H]⁺; rt: 1.65 min (LC/MS-method A). P2: LC/MS: m/z=174.0 [M+H]⁺; rt: 1.29 min (LC/MS-method A).

### Step 2: 1-(Ethyl-d5)-1H-pyrazole-5-carboxylic acid (P1)

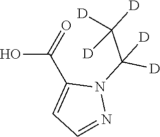 [SMILES: [2H]C([2H])([2H])C([2H])([2H])n1nccc1C(=O)O]

Ethyl 1-(ethyl-d5)-1H-pyrazole-5-carboxylate (Step 1-P1, 186 mg) was dissolved in THE/water (4 ml/1 ml) and LiOH (51 mg) was added with stirring. After 5.5 h the mixture was poured onto brine and the pH was adjusted to 2-3 with HCl. EA was added and the aqueous phase was extracted with EA (3×). The combined organic fractions were dried over Na₂SO₄, filtered and concentrated in vacuo. The residue was dissolved in water/ACN and lyophilised overnight to yield 118 mg of the title compound. LC/MS: m/z=146.0 [M+H]⁺; rt: 0.83 min (LC/MS-method A).

### Step 2: 1-(Ethyl-d5)-1H-pyrazole-3-carboxylic acid (P2)

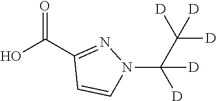 [SMILES: [2H]C([2H])([2H])C([2H])([2H])n1ccc(C(=O)O)n1]

Ethyl 1-(ethyl-d5)-1H-pyrazole-3-carboxylate (Step 1-P2, 259 mg) was analogously treated to P1 to yield 191 mg of the title compound. LC/MS: m/z=146.0 [M+H]⁺; rt: 0.74 min (LC/MS-method A).

### Intermediate 54: (S)-2-Amino-3,3-dicyclopropyl-N-(4-((S)-2-methoxy-1-((S)-2-oxo-4-(trifluoromethyl)imidazolidin-1-yl)ethyl)pyridin-2-yl)propanamide hydrochloride

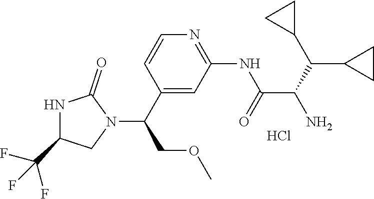 [SMILES: COC[C@H](c1ccnc(NC(=O)[C@@H](N)C(C2CC2)C2CC2)c1)N1C[C@@H](C(F)(F)F)NC1=O ; Cl]

### Step A-1: (2-Methoxyethene-1,1-diyl)dicyclopropane

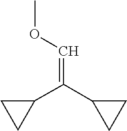

To a solution of (methoxymethyl)triphenylphosphonium chloride (202.3 g) in THE (1 l) was dropwise added n-BuLi (2.5 M, 236 ml) with stirring at 5° C. over 0.5 h. Then a solution of dicyclopropylmethanone (50 g) in THE (250 ml) was added to the mixture. The mixture was stirred at 5° C. for 0.5 h and then stirred at 60° C. for 16 h. The reaction solution was quenched with sat. aqueous NH₄Cl (500 ml) and then extracted with EA (300 ml, 2×). The combined organic layers were dried over Na₂SO₄, filtered and concentrated in vacuo to give a residue, which was purified by column chromatography (SiO₂, PE/EA=1/0 to 20/1) to yield 59 g of the title compound.

### Step A-2: 2,2-Dicyclopropylacetaldehyde

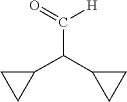

To a solution of (2-methoxyethene-1,1-diyl)dicyclopropane (57 g) in THE (300 ml) was added HCl (3 M, 302 ml). The mixture was stirred at 55° C. for 4 h. Then the reaction mixture was diluted with water (200 ml) and extracted with EA (150 ml, 3×). The combined organic layers were dried with anhydrous Na₂SO₄, filtered and concentrated under reduced pressure to yield 43 g of the crude title compound.

### Step A-3: 5-(Dicyclopropylmethyl)imidazolidine-2,4-dione

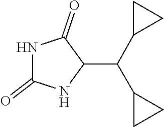 [SMILES: O=C1NC(=O)C(C(C2CC2)C2CC2)N1]

To a solution of 2,2-dicyclopropylacetaldehyde (43 g) in a mixed solution of EtOH (430 ml) and H₂O (430 ml) was added KCN (32.4 g) and (NH₄)₂CO₃ (99.8 g). The mixture was stirred at 75° C. for 8 h. Then the EtOH was removed under reduced pressure and then the residue was diluted with EA (200 ml) and extracted with EA (200 ml, 3×). The combined organic layers were washed with brine (400 ml), dried over anhydrous Na₂SO₄, filtered and concentrated under reduced pressure to give a crude product that was triturated with a mixture of PE and EA (PE:EA=5:1; 50 ml) at 25° C. for 20 min to yield 45 g of the title compound. ¹H NMR (400 MHz, CDCl₃) δ ppm: 8.78 (br s, 1H), 6.71 (br s, 1H), 4.26 (d, J=1.6 Hz, 1H), 0.88-0.69 (m, 3H), 0.68-0.59 (m, 1H), 0.56-0.44 (m, 2H), 0.44-0.30 (m, 2H), 0.28-0.17 (m, 3H).

### Step A-4: 2-((Tert-butoxycarbonyl)amino)-3,3-dicyclopropylpropanoic acid

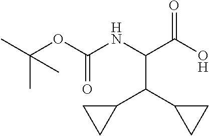 [SMILES: CC(C)(C)OC(=O)NC(C(=O)O)C(C1CC1)C1CC1]

To a mixture of 5-(dicyclopropylmethyl)imidazolidine-2,4-dione (20 g) in H₂O (400 ml) was added NaOH (2.5 M, 412 ml) and then stirred at 110° C. for 12 h. Then THF (400 ml) and Boc₂O (33.70 g) were added. The mixture was stirred at 25° C. for 12 h. Then H₂O (190 ml) was added and the aqueous phase was extracted with EA/PE (1:1, 200 ml, 2×). The aqueous layer was acidified with 1 N HCl to pH=4~5 and extracted with EA (200 ml, 3×). The combined organic phases were washed with brine (100 ml), dried over anhydrous Na₂SO₄, filtered and concentrated in vacuo to yield 15.22 g of the crude title compound.

### Step A-5: 4-Methoxybenzyl (S)-2-((tert-butoxycarbonyl)amino)-3,3-dicyclopropylpropanoate

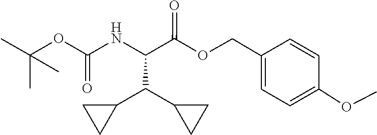 [SMILES: COc1ccc(COC(=O)[C@@H](NC(=O)OC(C)(C)C)C(C2CC2)C2CC2)cc1]

To a solution of 2-((tert-butoxycarbonyl)amino)-3,3-dicyclopropylpropanoic acid (15.22 g), (4-methoxyphenyl)methanol and DMAP (6.90 g) in DCM (230 ml) was added EDCl (16.25 g). The mixture was stirred at 25° C. for 16 h. Then the reaction mixture was washed with brine (200 ml). The organic layer was dried over anhydrous Na₂SO₄, filtered and concentrated under reduced pressure to give a residue, which was purified by column chromatography (SiO₂, PE/EA=1:0 to 10:1) to yield 15 g of the racemic title compound. ¹H NMR (400 MHz, CDCl₃) δ ppm: 7.30 (br d, J=8.5 Hz, 2H), 6.89 (d, J=8.8 Hz, 2H), 5.25 (br d, J=9.0 Hz, 1H), 5.09 (s, 2H), 4.62-4.48 (m, 1H), 3.82 (s, 3H), 1.45 (s, 9H), 0.78-0.59 (m, 3H), 0.54-0.39 (m, 3H), 0.36-0.28 (m, 1H), 0.24-0.12 (m, 3H), 0.02 (qd, J=4.9, 9.4 Hz, 1H).

The enantiomers (15 g) were separated by preparative SFC (column: Daicel Chiralpak AY (250 mm×50 mm, 10 μm); mobile phase: A: CO₂, B: [0.1% NH₃·H₂O in IPA]; B: 50%, isocratic mode) to yield 6.7 g of the title compound. ¹H NMR (400 MHz, CDCl₃) δ ppm: 7.30 (br d, J=8.5 Hz, 2H), 6.89 (d, J=8.6 Hz, 2H), 5.26 (br d, J=8.6 Hz, 1H), 5.09 (s, 2H), 4.54 (br dd, J=2.4, 9.1 Hz, 1H), 3.82 (s, 3H), 1.45 (s, 9H), 0.79-0.58 (m, 3H), 0.55-0.38 (m, 3H), 0.37-0.28 (m, 1H), 0.24-0.12 (m, 3H), 0.01 (qd, J=4.8, 9.5 Hz, 1H). Analytical SFC data (Chiralpak AY-3, 50×4.6 mm I.D., 3 μm); A: CO₂, B: IPA (0.05% DEA)]; B %: 5%-40%, flow 3 ml/min, T: 35° C.): peak 1 (R): RT=0.94 min; peak 2 (S): RT=1.22 min.

### Step A-5: (S)-2-((Tert-butoxycarbonyl)amino)-3,3-dicyclopropylpropanoic acid

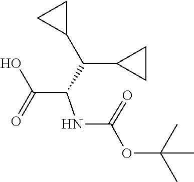 [SMILES: CC(C)(C)OC(=O)N[C@H](C(=O)O)C(C1CC1)C1CC1]

To a solution of 4-methoxybenzyl (S)-2-((tert-butoxycarbonyl)amino)-3,3-dicyclopropylpropanoate (10 g) in MeOH (50 ml) was added Pd/C (0.4 g, 10%) under N₂ atmosphere. The suspension was degassed and purged with H₂ for 3 times. The mixture was stirred under H₂ (15 psi) at 30° C. for 16 h. Then the mixture was filtered. After evaporation of the solvent in vacuo the residue was purified by column chromatography (SiO₂, PE/EA=1/0 to 5/1) to yield 6.5 g of the title compound. ¹H NMR (400 MHz, CDCl₃) δ ppm: 12.72-12.11 (m, 1H), 6.84 (br d, J=9.0 Hz, 1H), 4.12 (dd, J=4.4, 8.9 Hz, 1H), 1.39 (s, 9H), 1.00-0.88 (m, 1H), 0.83-0.72 (m, 1H), 0.55-0.41 (m, 2H), 0.40-0.20 (m, 4H), 0.18-0.00 (m, 3H).

### Step B-1: N,2-Dimethoxy-N-methylacetamide

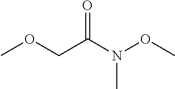

To a solution of N,O-dimethylhydroxylamine (100 g) and 2-methoxyacetyl chloride (37.09 g) in DCM (1 l) was added TEA (190 ml) at 0° C. The mixture was stirred at 25° C. for 1 h. Then the solution was treated with HCl (1 M) to pH=2~3. The organic phase was washed with brine, dried over anhydrous Na₂SO₄, filtered and concentrated in vacuo to yield 82.1 g of the title compound. ¹H NMR (400 MHz, CDCl₃) δ ppm: 4.20 (s, 2H), 3.67 (s, 3H), 3.45 (s, 3H), 3.17 (s, 3H).

### Step B-2: 1-(2-Chloropyridin-4-yl)-2-methoxyethan-1-one

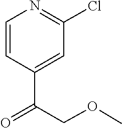

To a solution of 2-chloro-4-iodopyridine (80.93 g) in THE (1 l) was added n-BuLi (2.5 M, 203 ml) at −78° C. and the mixture was stirred for 10 min. Then N,2-dimethoxy-N-methylacetamide (45 g) was added at −78° C. over 1 h. After stirring the mixture at −78° C. for 2 h the reaction mixture was quenched by addition of sat. NHCl₄ solution (200 ml) at 0° C., diluted with water (200 ml) and extracted with EA (150 ml, 3×). The combined organic layers were washed with brine (100 ml, 2×), dried over anhydrous Na₂SO₄, filtered and concentrated under reduced pressure to give a residue which was purified by column chromatography (SiO₂, PE/EA=1/0 to 3/1) to yield 20 g of the title compound. LC/MS: m/z=186.2 [M+H]⁺; rt: 0.23 min (LC/MS-method C).

### Step B-3: Tert-butyl (4-(2-methoxyacetyl)pyridin-2-yl)carbamate

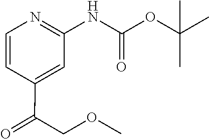 [SMILES: COCC(=O)c1ccnc(NC(=O)OC(C)(C)C)c1]

To a solution of 1-(2-chloropyridin-4-yl)-2-methoxyethan-1-one (20 g) and NH₂Boc (18.93 g) in dioxane (400 ml) were added Pd(OAc)₂ (1.21 g), XPhos (2.57 g) and Cs₂CO₃ (52.66 g). The mixture was stirred at 90° C. for 12 h. Then the reaction mixture was filtered and concentrated under reduced pressure to give a residue which was purified by column chromatography (SiO₂, PE/EA=1/0 to 3/1) to yield 8.1 g of the title compound.

### Step B-4: Tert-butyl (4-(1-(((S)-2-amino-3,3,3-trifluoropropyl)amino)-2-methoxyethyl)pyridin-2-yl)carbamate

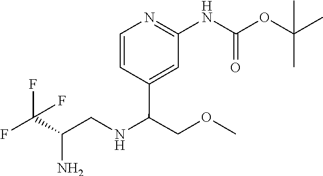 [SMILES: COCC(NC[C@H](N)C(F)(F)F)c1ccnc(NC(=O)OC(C)(C)C)c1]

To a solution of tert-butyl (4-(2-methoxyacetyl)pyridin-2-yl)carbamate (6 g) and (S)-3,3,3-trifluoropropane-1,2-diamine dihydrochloride (Int. 1, Step B6, 3.17 g) in IPA (140 ml) was added TEA (11.29 ml) in one portion at 85° C. and the mixture was stirred for 2 h at 85° C. After stirring for 12 h at 25° C. the mixture is concentrated in vacuo and the residue was dissolved in MeOH (140 ml). NaBH₃CN (7.93 g) was portion wise added, with gas evolution occurring. The suspension was stirred for 5 min, then AcOH (9.29 ml) was added. The reaction mixture was heated at 40° C. with stirring for 25 min. Then the reaction mixture was concentrated, diluted with DCM (100 ml) and stirred rapidly with saturated aqueous NaHCO₃ solution (200 ml) at 25° C. for 1 h. After the layers were separated, the aqueous phase was extracted with DCM (200 ml, 3×). The combined organic layers were washed with brine (300 ml), dried over anhydrous Na₂SO₄, filtered and concentrated to yield 10 g of the crude title compound. LC/MS: m/z=379.3 [M+H]⁺; rt: 0.24 min (LC/MS-method C).

### Step B-5-P1 and -P2: Tert-butyl (4-((S)-2-methoxy-1-((S)-2-oxo-4-(trifluoromethyl)imidazolidin-1-yl)ethyl)pyridin-2-yl)carbamate and tert-butyl (4-((R)-2-methoxy-1-((S)-2-oxo-4-(trifluoromethyl)imidazolidin-1-yl)ethyl)pyridin-2-yl)carbamate

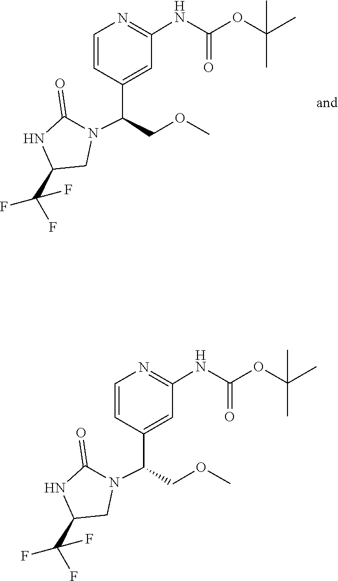 [SMILES: COC[C@H](c1ccnc(NC(=O)OC(C)(C)C)c1)N1C[C@@H](C(F)(F)F)NC1=O ; COC[C@@H](c1ccnc(NC(=O)OC(C)(C)C)c1)N1C[C@@H](C(F)(F)F)NC1=O]

To a solution of tert-butyl (4-(1-(((S)-2-amino-3,3,3-trifluoropropyl)amino)-2-methoxyethyl)pyridin-2-yl)carbamate (10 g) in THE (240 ml) was added CDI (12.86 g). The mixture was stirred at 60° C. for 1 h. Then the mixture was diluted with water (150 ml) and extracted with EA (150 ml, 3×). The combined organic layers were washed with citric acid (100 ml, 2×), dried over anhydrous Na₂SO₄, filtered and concentrated under reduced pressure to give a residue, which was purified by column chromatography (SiO₂, PE/EA=I/O to 0/1) to yield 2.5 g of the diastereomeric mixture of title compound.

The diastereomers (5.5 g) were separated by preparative SFC (column: Regis (S,S) Whelk-01 (250 mm×50 mm, 10 μm); mobile phase: A: CO₂, B: [0.1% NH₃·H₂O in MeOH]; B: 25%-isocratic) to yield 2.8 g of B-5-P1 and 2 g of B-5-P2. P1: LC/MS: m/z=405.3 [M+H]⁺; rt: 0.31 min (LC/MS-method C). P2: LC/MS: m/z=405.3 [M+H]⁺; rt: 0.32 min (LC/MS-method C). Analytical SFC data: (Regis, Whelk (50×4.6 mm, 3.5 μm); A: CO₂, B: MeOH (0.05% DEA)]; B %: 5%-40%, flow 3 ml/min, T: 35° C.): P1: RT=1.16 min; P2: RT=1.27 min.

### Step B-6-P1: (S)-1-((S)-1-(2-Aminopyridin-4-yl)-2-methoxyethyl)-4-(trifluoromethyl)imidazolidin-2-one hydrochloride

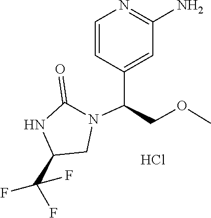 [SMILES: COC[C@H](c1ccnc(N)c1)N1C[C@@H](C(F)(F)F)NC1=O ; Cl]

To a solution of tert-butyl (4-((S)-2-methoxy-1-((S)-2-oxo-4-(trifluoromethyl)imidazolidin-1-yl)ethyl)pyridin-2-yl)carbamate (2.8 g) was added HCl/dioxane (4 M, 10.39 ml). The mixture was stirred at 25° C. for 1 h. Then the reaction mixture was filtered and concentrated under reduced pressure to yield 1.9 g of the title compound. Analytical SFC data: (Chiralpak IC-3 (50×4.6 mm, 3 μm); A: CO₂, B: MeOH (0.05% DEA)]; B %: 5%-40%, flow 3 ml/min, T: 35° C.): P1: RT=1.56 min.

### Step B-6-P2: (S)-1-((R)-1-(2-aminopyridin-4-yl)-2-methoxyethyl)-4-(trifluoromethyl)imidazolidin-2-one hydrochloride

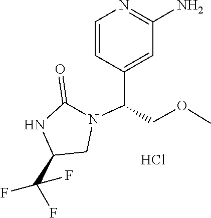 [SMILES: COC[C@@H](c1ccnc(N)c1)N1C[C@@H](C(F)(F)F)NC1=O ; Cl]

Following the procedure described for B-6-P1 tert-butyl (4-((R)-2-methoxy-1-((S)-2-oxo-4-(trifluoromethyl)imidazolidin-1-yl)ethyl)pyridin-2-yl)carbamate (2 g) yielded 1.4 g of the title compound. Analytical SFC data: (Chiralpak IC-3 (50×4.6 mm, 3 μm); A: CO₂, B: MeOH (0.05% DEA)]; B %: 5%-40%, flow 3 ml/min, T: 35° C.): P1: RT=1.76 min.

### Step 1: Tert-butyl ((S)-1,1-dicyclopropyl-3-((4-((S)-2-methoxy-1-((S)-2-oxo-4-(trifluoromethyl)imidazolidin-1-yl)ethyl)pyridin-2-yl)amino)-3-oxopropan-2-yl)carbamate (DS2)

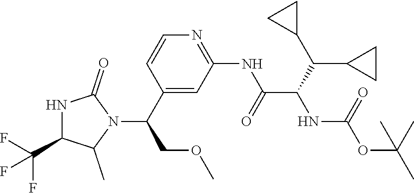 [SMILES: COC[C@H](c1ccnc(NC(=O)[C@@H](NC(=O)OC(C)(C)C)C(C2CC2)C2CC2)c1)N1C(=O)N[C@H](C(F)(F)F)C1C]

To (S)-2-((tert-butoxycarbonyl)amino)-3,3-dicyclopropylpropanoic acid (Step A-5, 100 mg) in DCM (2.8 ml) were added DMAP (91 mg) and EDCl (142 mg). After stirring for 5 min (S)-1-((S)-1-(2-aminopyridin-4-yl)-2-methoxyethyl)-4-(trifluoromethyl)imidazolidin-2-one hydrochloride (Step B-6-P1, 113 mg) was added. After stirring for 1 h the mixture stood overnight. It was poured into sat. NaHCO₃ solution and the aqueous phase was extracted with DCM (2×). The combined organic phases were dried over Na₂SO₄, filtered and concentrated in vacuo. The residue was purified by silica gel chromatography (12 g silica gel; DCM/EtOH: 100:0 for 5 min, 100:0 to 92:8 in 30 min, 92:8 for 10 min). The pure compound containing fractions were combined and concentrated in vacuo to yield 125 mg of a diastereomeric mixture.

The diastereomeric mixture (120 mg) was separated by preparative SFC chromatography (Chiralpak IE/67 (30×250 mm, 5 μM); A CO₂ B: MeOH (35%); flow: 120 ml/min; T: 40° C.) to yield 24 mg of DS 1 and 69 mg of DS 2, the title compound. Analytical chiral analysis (HPLC; Chiralpak IE/179, 4.6×250 mm, 5 μM; MeOH/EtOH 1:1, flow: 1.0 ml/min; T: 30° C.): DS 1: rt: 4.19 min. DS 2: 4.80 min.

### Step 2: (S)-2-Amino-3,3-dicyclopropyl-N-(4-((S)-2-methoxy-1-((S)-2-oxo-4-(trifluoromethyl)imidazolidin-1-yl)ethyl)pyridin-2-yl)propanamide hydrochloride

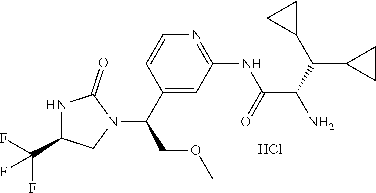 [SMILES: COC[C@H](c1ccnc(NC(=O)[C@@H](N)C(C2CC2)C2CC2)c1)N1C[C@@H](C(F)(F)F)NC1=O ; Cl]

Tert-butyl ((S)-1,1-dicyclopropyl-3-((4-((S)-2-methoxy-1-((S)-2-oxo-4-(trifluoromethyl)imidazolidin-1-yl)ethyl)pyridin-2-yl)amino)-3-oxopropan-2-yl)carbamate (Step 1, 60 mg) was dissolved in dry dioxane (2.0 ml) and HCl solution (2.0 ml; 4 M in dioxane) was added dropwise with stirring. After 2 h the mixture was concentrated in vacuo and the residue was lyophilized from ACN/H₂O to yield 58 mg of the crude title compound. LC/MS: m/z=456.2 [M+H]⁺; rt: 1.19 min (LC/MS-method A).

### Intermediate 55: (2S)-2-Amino-2-((1r,4S)-4-methylcyclohexyl)-N-(4-(((S)-2-oxo-4-(trifluoromethyl)imidazolidin-1-yl)methyl-d)pyridin-2-yl)acetamide hydrochloride

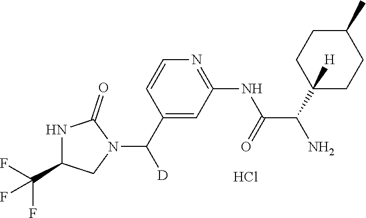

### Step 1: Tert-butyl ((1S)-2-((4-((((S)-2-amino-3,3,3-trifluoropropyl)amino)methyl-d)pyridin-2-yl)amino)-1-((1r,4S)-4-methylcyclohexyl)-2-oxoethyl)carbamate

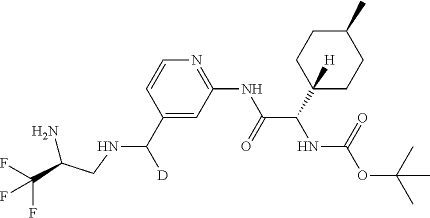

Tert-butyl ((S)-2-((4-formylpyridin-2-yl)amino)-1-((1r,4S)-4-methylcyclohexyl)-2-oxoethyl)carbamate (Int. 1, Step A4, 120 mg) and (S)-3,3,3-trifluoropropane-1,2-diamine dihydrochloride (Int. 1, Step B6, 77 mg) were dissolved in DCM (8 ml) under Ar. After the addition of TEA (196 μl) the mixture was stirred for 10 min. Then the mixture was heated to 40° C. for 3.5 h. Then NaBD₃CN (74 mg), dry MeOH (33 μl) and AcOH (95 μl) were added at RT and the mixture was stirred for 20 min. After standing overnight DCM and sat. NaHCO₃ solution were added. After separation of the organic phase the aqueous phase was extracted with DCM (2×). The combined organic phases were dried over Na₂SO₄, filtered and concentrated in vacuo. The residue was further purified by preparative RP HPLC (flow: 25 ml/min; gradient: 95% H₂O+0.05% TFA/5% ACN in 45 min to 10% H₂O+0.05% TFA/90% ACN; column: Purosphere® STAR-RP18, 25 mm×250 mm, 10 μm). The pure product containing fractions were combined, the ACN was partly removed and the TFA neutralised by concentrated NaHCO₃ solution. After extracting the product with DCM (3×) the combined organic phases were dried over sodium sulphate, filtered and concentrated in vacuo. The residue was dissolved in ACN/H₂O and freeze dried to yield 96 mg of the title compound. LC/MS: m/z=489.3 [M+H]⁺; rt: 1.70 min (LC/MS-method A).

### Step 2: Tert-butyl ((1S)-1-((1r,4S)-4-methylcyclohexyl)-2-oxo-2-((4-(((S)-2-oxo-4-(trifluoromethyl)imidazolidin-1-yl)methyl-d)pyridin-2-yl)amino)ethyl)carbamate

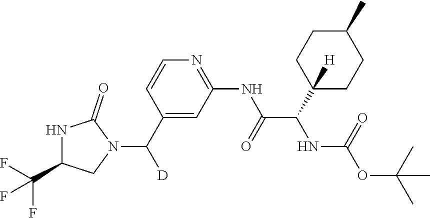

Tert-butyl ((1S)-2-((4-((((S)-2-amino-3,3,3-trifluoropropyl)amino)methyl-d)pyridin-2-yl)amino)-1-((1r,4S)-4-methylcyclohexyl)-2-oxoethyl)carbamate (Step 1, 95 mg) was dissolved in THE (5 ml) and heated to 65° C. for 5 min. Then CDI (95 mg) was added and the mixture was heated for 5.5 h with stirring. After standing overnight at RT heating was continued at 65° C. for 2 h. After cooling DCM was added and the mixture was washed with sat. NaHCO₃ solution. This aqueous phase was extracted with DCM (2×). The combined organic phases were dried over Na₂SO₄, filtered and concentrated in vacuo. The residue was purified by silica gel chromatography (silica gel 12 g, DCM/EtOH gradient). Product containing pure fractions were combined and concentrated in vacuo to yield 86 mg of the title compound. LC/MS: m/z=515.3 [M+H]⁺; rt: 2.33 min (LC/MS-method A).

### Step 3: (2S)-2-Amino-2-((1r,4S)-4-methylcyclohexyl)-N-(4-(((S)-2-oxo-4-(trifluoromethyl)imidazolidin-1-yl)methyl-d)pyridin-2-yl)acetamide hydrochloride

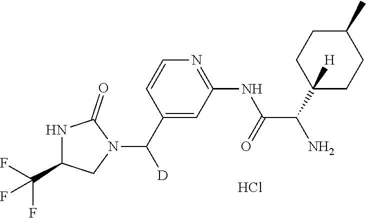

Tert-butyl ((1S)-1-((1r,4S)-4-methylcyclohexyl)-2-oxo-2-((4-(((S)-2-oxo-4-(trifluoromethyl)imidazolidin-1-yl)methyl-d)pyridin-2-yl)amino)ethyl)carbamate (Step 2, 81 mg) was dissolved in HCl (4 N in dioxane, 2 ml). After 1.75 h the solvent was removed in vacuo and the residue was lyophilised from ACN/water to yield 78 mg of the title compound. LC/MS: m/z=415.2 [M+H]⁺; rt: 1.27 min (LC/MS-method A).

### Intermediate 56-DS1 and DS2: (2S)-2-Amino-N-(4-((R or S)-2-hydroxy-1-((S)-2-oxo-4-(trifluoromethyl)imidazolidin-1-yl)ethyl)pyridin-2-yl)-2-((1r,4S)-4-methylcyclohexyl)acetamide hydrochloride

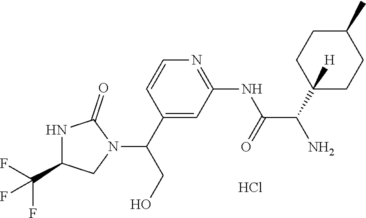

### Step 1: Tert-butyl ((S)-2-((4-bromopyridin-2-yl)amino)-1-((1r,4S)-4-methylcyclohexyl)-2-oxoethyl)carbamate

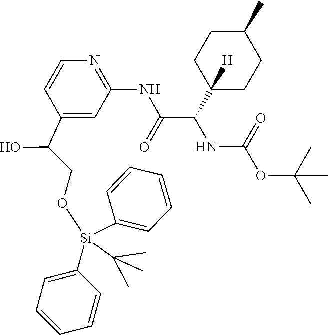

To a mixture of (S)-2-((tert-butoxycarbonyl)amino)-2-((1r,4S)-4-methylcyclohexyl)acetic acid (6 g) and 4-bromopyridin-2-amine (5.74 g) in pyridine (60 ml) was added T3P (19.73 ml) in one portion at −20° C. under N₂. The mixture was stirred at 20° C. for 12 h. Then the reaction mixture was partitioned between water (120 ml) and EA (120 ml). The organic phase was separated, dried over Na₂SO₄, filtered and concentrated under reduced pressure to give a residue which was purified by column chromatography (SiO₂, PE/DCM/EA=16/4/1) to yield 4.1 g of the title compound. LC/MS: m/z=426.2 [M+H]⁺; rt: 0.50 min (LC/MS-method C).

### Step 2: Tert-butyl ((S)-1-((1r,4S)-4-methylcyclohexyl)-2-oxo-2-((4-vinylpyridin-2-yl)amino)ethyl)carbamate

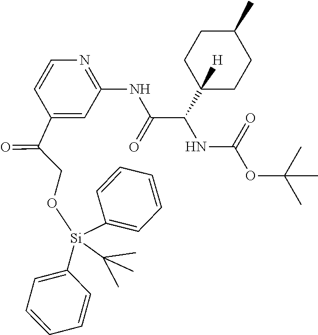

To tert-butyl ((S)-2-((4-bromopyridin-2-yl)amino)-1-((1r,4S)-4-methylcyclohexyl)-2-oxoethyl)carbamate (750 mg), potassium vinyltrifluoroborate (353 mg), K₃PO₄ (1.12 g) and Pd(dtpbf)Cl₂ (229 mg) were added dioxane (16 ml) and water (3.5 ml). The mixture was purged with Ar for 15 min and then stirred at 100° C. for 30 min. The mixture was poured into brine and the aqueous phase was extracted with EA (2×). The combined organic phases were dried over Na₂SO₄, filtered and concentrated in vacuo. The residue was purified by silica gel chromatography (silica gel 24 g, Hep/EA: 100% Hep (5 min), to 100% EA in 45 min, 100% EA for 10 min). Product containing pure fractions were combined and concentrated in vacuo to yield 517 mg of the title compound. LC/MS: m/z=374.2 [M+H]⁺; rt: 2.62 min (LC/MS-method A).

### Step 3: Tert-butyl ((1S)-2-((4-(1,2-dihydroxyethyl)pyridin-2-yl)amino)-1-((1r,4S)-4-methylcyclohexyl)-2-oxoethyl)carbamate

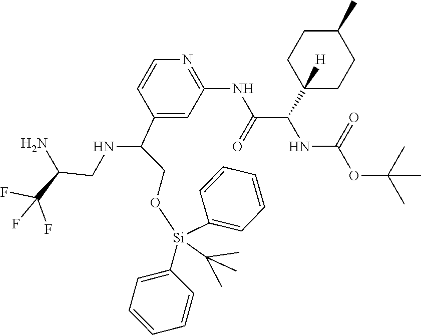

To tert-butyl ((S)-1-((1r,4S)-4-methylcyclohexyl)-2-oxo-2-((4-vinylpyridin-2-yl)amino)ethyl)carbamate (510 mg) were added dioxane (6 ml) and water (3 ml). Then 4-methylmorpholine N-oxide (420 mg) and potassium osmate(VI) dihydrate (14 mg) were added with stirring. After stirring for 2 h at 40° C. sat. NaHCO₃ solution (5 ml) was added and the mixture was stirred for 15 min. Then water and EA were added and the mixture was filtered. The residue was washed with EA and water. Brine was added to the filtrate and the aqueous phase was extracted with EA (3×). The combined organic phases were dried over Na₂SO₄, filtered and concentrated in vacuo. The residue was purified by silica gel chromatography (Silica gel 12 g, 100% DCM (for 5 min), to 85% DCM/15% EtOH in 45 min, 15% EtOH for 5 min). Product containing pure fractions were combined and concentrated in vacuo to yield 421 mg of the title compound. LC/MS: m/z=408.2 [M+H]⁺; rt: 1.91 min (LC/MS-method A).

### Step 4: Tert-butyl ((1S)-2-((4-(2-((tert-butyldiphenylsilyl)oxy)-1-hydroxyethyl)pyridin-2-yl)amino)-1-((1r,4S)-4-methylcyclohexyl)-2-oxoethyl)carbamate

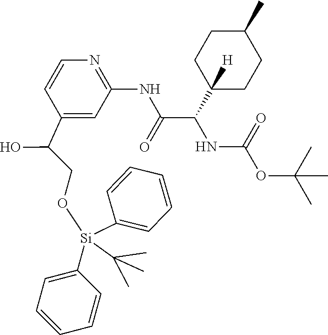

Tert-butyl ((1S)-2-((4-(1,2-dihydroxyethyl)pyridin-2-yl)amino)-1-((1r,4S)-4-methylcyclohexyl)-2-oxoethyl)carbamate (420 mg) was dissolved in DCM and DMAP (13 mg), TEA (180 μl) and tert-butylchlorodiphenylsilane (320 μl) were added with stirring at RT. After stirring for 2 h the mixture stood overnight before additional silane (53 μl) was added. After standing for 4 days further silane (266 μl) was added and the mixture was stirred for 4 h at 35° C. After standing overnight the mixture was poured into brine and the aqueous phase was extracted with DCM (2×). The combined organic phases were dried over Na₂SO₄, filtered and concentrated in vacuo. The residue was purified by silica gel chromatography (silica gel 24 g, nHep/EA: 100% Hep (5 min), to 50/50 in 30 min, to 0/100 20 min). Product containing pure fractions were combined and concentrated in vacuo to yield 382 mg of the title compound. LC/MS: m/z=644.4 [M+H]⁺; rt: 3.56 min (LC/MS-method A).

### Step 5: Tert-butyl ((S)-2-((4-(2-((tert-butyldiphenylsilyl)oxy)acetyl)pyridin-2-yl)amino)-1-((1r,4S)-4-methylcyclohexyl)-2-oxoethyl)carbamate

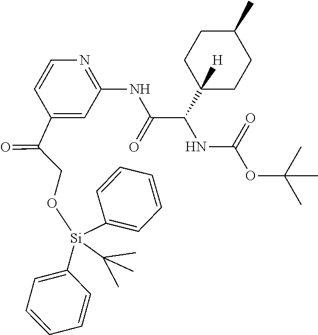

Tert-butyl ((1S)-2-((4-(2-((tert-butyldiphenylsilyl)oxy)-1-hydroxyethyl)pyridin-2-yl)amino)-1-((1r,4S)-4-methylcyclohexyl)-2-oxoethyl)carbamate (380 mg) was dissolved in dry DCM and DMP (15% in DCM, 1.47 ml) was added with stirring at RT. After stirring for 2 h and standing overnight further DMP (0.244 ml) was added. After standing overnight the mixture was poured into water and brine was added. The aqueous phase was extracted with DCM (3×) and the combined organic phases were dried over Na₂SO₄, filtered and concentrated in vacuo. The residue was purified by silica gel chromatography (silica gel 12 g, nHep/EA: 100% nHep (5 min), to 60/40 in 30 min, 60/40 for 10 min). Product containing pure fractions were combined and concentrated in vacuo to yield 321 mg of the title compound. LC/MS: m/z=644.4 [M+H]⁺; rt: 3.56 min (LC/MS-method A).

### Step 6: Tert-butyl ((1S)-2-((4-(1-(((S)-2-amino-3,3,3-trifluoropropyl)amino)-2-((tert-butyldiphenylsilyl)oxy)ethyl)pyridin-2-yl)amino)-1-((1r,4S)-4-methylcyclohexyl)-2-oxoethyl)carbamate

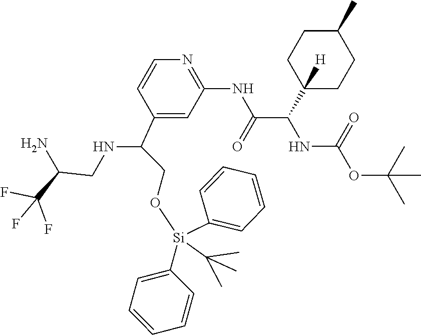

Tert-butyl ((S)-2-((4-(2-((tert-butyldiphenylsilyl)oxy)acetyl)pyridin-2-yl)amino)-1-((1r,4S)-4-methylcyclohexyl)-2-oxoethyl)carbamate (Step 5, 320 mg) and (S)-3,3,3-trifluoropropane-1,2-diamine dihydrochloride (Int. 1, Step B6, 119 mg) were dissolved in DCM (8 ml) under Ar. After the addition of TEA (0.3 ml) the mixture was stirred for 10 min. Then the mixture was heated to 40° C. for 1 h. Then NaBD₃CN (109 mg), dry MeOH (5 ml) and AcOH (150 μl) were added at RT and the mixture was stirred over the weekend. Then the mixture was poured into and sat. NaHCO₃ solution and the aqueous phase was extracted with DCM (3×). The combined organic phases were dried over Na₂SO₄, filtered and concentrated in vacuo. The residue was further purified by preparative RP HPLC (flow: 25 ml/min; gradient: for 20 min 100% H₂O+0.05% TFA; then in 30 min to 55% H₂O+0.05% TFA/45% ACN; column: Purosphere® STAR-RP18, 25 mm×250 mm, 10 μm). The pure product containing fractions were combined, the ACN was partly removed and the TFA neutralised with conc. NaHCO₃ solution. After extracting the product with DCM (2×) the combined organic phases were dried over sodium sulphate, filtered and concentrated in vacuo. The residue was dissolved in ACN/H₂O and freeze dried to yield 124 mg of the title compound. LC/MS: m/z=756.2 [M+H]⁺; rt: 3.06 min (LC/MS-method A).

### Step 7: Tert-butyl ((1S)-2-((4-(2-((tert-butyldiphenylsilyl)oxy)-1-((S)-2-oxo-4-(trifluoromethyl)imidazolidin-1-yl)ethyl)pyridin-2-yl)amino)-1-((1r,4S)-4-methylcyclohexyl)-2-oxoethyl)carbamate

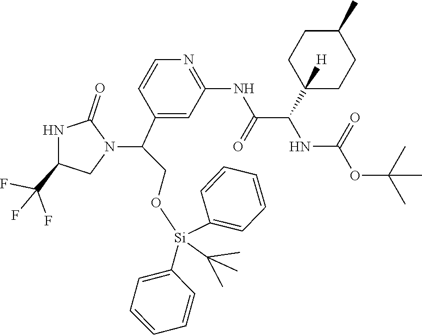

Tert-butyl ((1S)-2-((4-(1-(((S)-2-amino-3,3,3-trifluoropropyl)amino)-2-((tert-butyldiphenylsilyl)oxy)ethyl)pyridin-2-yl)amino)-1-((1r,4S)-4-methylcyclohexyl)-2-oxoethyl)carbamate (Step 6,123 mg) was dissolved in THE (5 ml) and heated to 65° C. with stirring. After 5 min CDI (79 mg) was added and stirring at 65° C. was continued for 23 h. After cooling the mixture was poured into sat. NaHCO₃ solution and the aqueous phase was extracted with DCM (2×). The combined organic phases were dried over Na₂SO₄, filtered and concentrated in vacuo. The residue was further purified by preparative RP HPLC (flow: 25 ml/min; gradient: 95% H₂O+0.05% TFA/5% ACN in 45 min to 10% H₂O+0.05% TFA/90% ACN; column: Purosphere® STAR-RP18, 25 mm×250 mm, 10 μm). The pure product containing fractions were combined, the ACN was partly removed and the TFA neutralised by sat. NaHCO₃ solution. After extracting the product with DCM (3×) the combined organic phases were dried over sodium sulphate, filtered and concentrated in vacuo. The residue was dissolved in ACN/H₂O and freeze dried to yield 81 mg of the title compound. LC/MS: m/z=782.3 [M+H]⁺; rt: 3.34 and 3.40 min (diastereomers) (LC/MS-method A).

### Step 8-P1 and -P2: Tert-butyl ((1S)-2-((4-((R or S)-2-hydroxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)carbamate

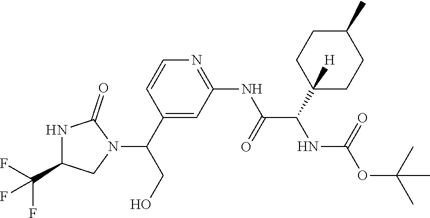

Tert-butyl ((1S)-2-((4-(2-((tert-butyldiphenylsilyl)oxy)-1-((S)-2-oxo-4-(trifluoromethyl)imidazolidin-1-yl)ethyl)pyridin-2-yl)amino)-1-((1r,4S)-4-methylcyclohexyl)-2-oxoethyl)carbamate (81 mg) was dissolved in THE (1.5 ml) and tetrabutylammonium fluoride (1 M in THF, 100 μl) was added with stirring. After 1.75 h the mixture was poured into brine and the aqueous phase was extracted with EA (2×). The combined organic phases were dried over Na₂SO₄, filtered and concentrated in vacuo. The residue was purified by silica gel chromatography (silica gel 4 g, 100% DCM for 5 min; 100% DCM to 90% DCM/10% EtOH in 30 min; hold 10% EtOH for 10 min). Product containing fractions were combined and lyophilised from water/ACN overnight to yield 49 mg of a diastereomeric mixture that was purified by preparative SFC (Chiralpak IA 32; 250×30 mm, 5 μm; A: CO₂, B: EtOH, 25% B isocratic mode; flow 120 ml/min) to yield 9 mg of pure P2, while P1 (16 mg) was still impure. P1 was further purified by preparative HPLC (column: XBridge BEH130, Prep C18, 10 μM, OBD (19×250 mm): flow: 15 ml/min; gradient: 95% H₂O+0.05% TFA/5% ACN in 45 min to 10% H₂O+0.05% TFA/90% ACN). The pure fractions were combined, the ACN was removed, pH adjusted to ~7 and the aqueous phase was extracted with DCM (2×). The combined organic phases were dried over Na₂SO₄, filtered and concentrated in vacuo. The residue was lyophilised from ACN/H₂O to yield 12 mg of P1. P1: LC/MS: m/z=544.2 [M+H]⁺; rt: 2.18 min (LC/MS-method A). P2: LC/MS: m/z=544.2 [M+H]⁺; rt: 2.19 min (LC/MS-method A).

### Step 9: (2S)-2-Amino-N-(4-((R or S)-2-hydroxy-1-((S)-2-oxo-4-(trifluoromethyl)-imidazolidin-1-yl)ethyl)pyridin-2-yl)-2-((1r,4S)-4-methylcyclohexyl)acetamide hydrochloride—DS1

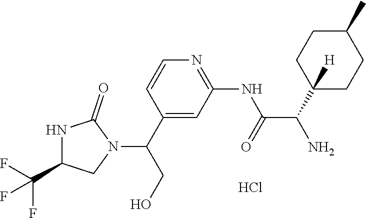

Tert-butyl ((1S)-2-((4-((R or S)-2-hydroxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)carbamate (Step 8-P1, 11 mg) was dissolved in dioxane (0.5 ml) and HCl (4 N in dioxane, 0.5 ml) was added. After 1.5 h the solvent was removed in vacuo and the residue was lyophilised from ACN/water to yield 11 mg of the title compound. LC/MS: m/z=444.1 [M+H]⁺; rt: 1.18 min (LC/MS-method A).

### Step 9: (2S)-2-Amino-N-(4-((R or S)-2-hydroxy-1-((S)-2-oxo-4-(trifluoromethyl)-imidazolidin-1-yl)ethyl)pyridin-2-yl)-2-((1r,4S)-4-methylcyclohexyl)acetamide hydrochloride—DS2

Tert-butyl ((1S)-2-((4-((R or S)-2-hydroxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)carbamate (Step 8-P2, 7 mg) was dissolved in dioxane (0.5 ml) and HCl (4 N in dioxane, 0.5 ml) was added. After 1 h the solvent was removed in vacuo and the residue was lyophilised from ACN/water to yield 6 mg of the title compound. LC/MS: m/z=444.3 [M+H]⁺; rt: 0.55 min (LC/MS-method B).

### Intermediate 57: N-(1-(2-((S)-2-amino-2-(4,4-difluorocyclohexyl)acetamido)pyridin-4-yl)-3,3,3-trifluoropropyl)-4,4,4-trifluorobutanamide

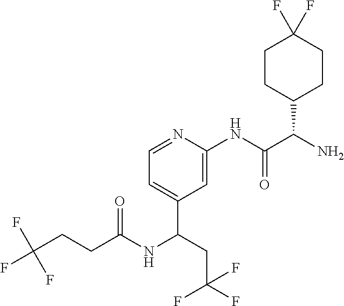 [SMILES: N[C@H](C(=O)Nc1cc(C(CC(F)(F)F)NC(=O)CCC(F)(F)F)ccn1)C1CCC(F)(F)CC1]

### Step 1: tert-butyl (4-vinylpyridin-2-yl)carbamate

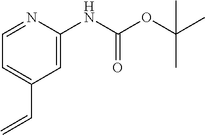 [SMILES: C=Cc1ccnc(NC(=O)OC(C)(C)C)c1]

To a solution of compound tert-butyl (4-bromopyridin-2-yl)carbamate (42 g) and potassium vinyltrifluoroborate (20.60 g) in dioxane (400 ml) and water (400 ml) was added Cs₂CO₃ (150.31 g) and Pd(dppf)Cl₂·CH₂Cl₂ (12.56 g). The mixture was stirred at 100° C. for 7 h before the reaction mixture was cooled and diluted with water (500 ml) and extracted with DCM (300 ml×3). The combined organic phases were dried over Na₂SO₄, filtered and concentrated under reduced pressure to give a residue that was purified by FC (SiO₂, PE/EA=1/0 to 10/1) to afford tert-butyl (4-vinylpyridin-2-yl)carbamate (17 g) as an off white solid. LC/MS: m/z=221.2 [M+H]⁺; rt: 0.70 min (LC/MS-method C).

### Step 2: tert-butyl (4-(1-chloro-3,3,3-trifluoropropyl)pyridin-2-yl)carbamate

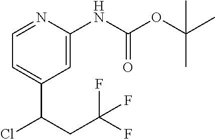 [SMILES: CC(C)(C)OC(=O)Nc1cc(C(Cl)CC(F)(F)F)ccn1]

To a solution of tert-butyl (4-vinylpyridin-2-yl)carbamate (6 g) in dioxane (70 ml) was added CuCl₂ (366.24 mg) and trifluoromethanesulfonyl chloride (13.77 g) in three parallel reactions that was stirred at 100° C. for 5 h. The reaction mixtures were filtered through a pad of Celite eluting with DCM and EA and the combined filtrate was concentrated in vacuo to afford a residue that was purified by FC (SiO₂, PE/EA=1/0 to 1/1). This afforded tert-butyl (4-(1-chloro-3,3,3-trifluoropropyl)pyridin-2-yl)carbamate (13 g) as a yellow solid. LC/MS: m/z=325.1 [M+H]⁺; rt: 0.75 min (LC/MS-method C).

### Step 3: tert-butyl (4-(1-azido-3,3,3-trifluoropropyl)pyridin-2-yl)carbamate

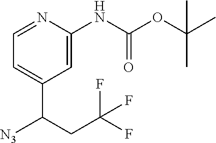 [SMILES: CC(C)(C)OC(=O)Nc1cc(C(CC(F)(F)F)N=[N+]=[N-])ccn1]

To a solution of tert-butyl (4-(1-chloro-3,3,3-trifluoropropyl)pyridin-2-yl)carbamate (12 g) in DMSO (180 ml) was added sodium azide (6.080 g) at RT and the mixture stirred at RT for 15 min under N₂ atmosphere before being heated to 60° C. for 1 h. After cooling the solution was diluted with Na₂CO₃ (sat. aq.) until pH>9 and then extracted with EA (300 ml×3). The combined organic phase was washed with brine (200 ml×3), dried over Na₂SO₄, filtered and concentrated in vacuo. The excess NaN₃ in the aqueous phase was quenched by NaClO (aq. 300 mL). tert-butyl (4-(1-azido-3,3,3-trifluoropropyl)pyridin-2-yl)carbamate (9.5 g) was obtained as a brown liquid that was used directly without further purification. LC/MS: m/z=276.0 [M+H]⁺; rt: 0.44 min (LC/MS-method C).

### Step 4: 4-(1-amino-3,3,3-trifluoropropyl)pyridin-2-amine

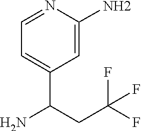 [SMILES: Nc1cc(C(N)CC(F)(F)F)ccn1]

To a solution of tert-butyl (4-(1-azido-3,3,3-trifluoropropyl)pyridin-2-yl)carbamate (10 g) in EA (200 ml) was added Pd/C (6 g) and the mixture was stirred at RT for 6 h under a hydrogen atmosphere. The reaction mixture was filtered and concentrated under reduced pressure to afford a residue that was diluted with HCl in EA (4 M, 77.79 ml) and the mixture was stirred at RT for 4 h before being concentrated under reduced pressure to afford 4-(1-amino-3,3,3-trifluoropropyl)pyridin-2-amine (11 g) as a yellow solid that was used with further purification. LC/MS: m/z=206.1 [M+H]⁺; rt: 0.38 min (LC/MS-method C).

### Step 5A: tert-butyl (1-(2-aminopyridin-4-yl)-3,3,3-trifluoropropyl)carbamate enantiomer 1 and Step 5B: tert-butyl (1-(2-aminopyridin-4-yl)-3,3,3-trifluoropropyl)carbamate Enantiomer 2

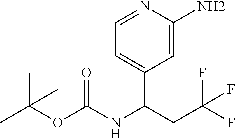 [SMILES: CC(C)(C)OC(=O)NC(CC(F)(F)F)c1ccnc(N)c1]

To a mixture of 4-(1-amino-3,3,3-trifluoropropyl)pyridin-2-amine (10 g), TEA (8.82 ml) in MeOH (100 ml) was added Boc₂O (9.57 g) in MeOH (100 ml) and purged with nitrogen 3 times before the mixture was stirred at 0° C. for 1 h under a nitrogen atmosphere. The reaction mixture was concentrated under reduced pressure to afford a residue that was purified by prep-HPLC (column: Phenomenex luna C18 (250*70 mm, 10 μm); mobile phase: [water (NH₄HCO₃)-ACN]; gradient: 45%-55% B over 22 min) to afford tert-butyl (1-(2-aminopyridin-4-yl)-3,3,3-trifluoropropyl)carbamate (4.5 g) as a yellow-brown solid. The enantiomers were separated by chiral SFC (column: DAICEL CHIRALPAK IC (250 mm*50 mm, 10 um); mobile phase: [CO₂-i-PrOH (0.1% NH₃H₂O)]; B %: 25%, isocratic elution mode) to afford Step 5A: tert-butyl (1-(2-aminopyridin-4-yl)-3,3,3-trifluoropropyl)carbamate enantiomer 1 (1.85 g) as a colorless solid. LC/MS: m/z=306.1 [M+H]⁺; rt: 0.26 min (LC/MS-method C); and Step 5B: tert-butyl (1-(2-aminopyridin-4-yl)-3,3,3-trifluoropropyl)carbamate enantiomer 2 (1.60 g) as a yellow solid. LC/MS: m/z=306.1 [M+H]⁺; rt: 0.27 min (LC/MS-method C).

### Step 6: tert-butyl (1-(2-((S)-2-(((benzyloxy)carbonyl)amino)-2-(4,4-difluorocyclohexyl)acetamido)pyridin-4-yl)-3,3,3-trifluoropropyl)carbamate

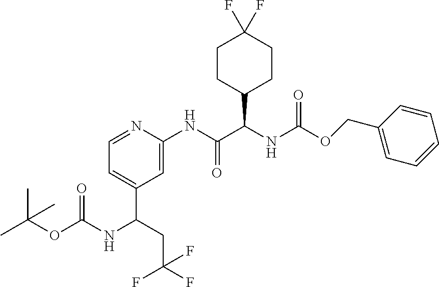 [SMILES: CC(C)(C)OC(=O)NC(CC(F)(F)F)c1ccnc(NC(=O)[C@H](NC(=O)OCc2ccccc2)C2CCC(F)(F)CC2)c1]

To a solution of compound tert-butyl (1-(2-aminopyridin-4-yl)-3,3,3-trifluoropropyl)carbamate enantiomer 2 (Step 5B, 1.35 g) and 2-(((benzyloxy)carbonyl)amino)-2-(4,4-difluorocyclohexyl)acetic acid (1.74 g) in pyridine (14 ml) was added EDCl (2.54 g) and the mixture was stirred at RT for 1 hr before the reaction mixture was concentrated under reduced pressure. The residue was purified by FC (SiO₂, PE/EA=1/0 to 2/1) to afford the title compound as a diastereomer mixture (2.4 g) as a colorless solid that was separated by chiral SFC (column: DAICEL CHIRALCEL OX (250 mm*30 mm, 10 um); mobile phase: [CO₂-i-PrOH (0.1% NH₃H₂O)]; B %: 15%, isocratic elution mode). This afforded tert-butyl (1-(2-((S)-2-(((benzyloxy)carbonyl)amino)-2-(4,4-difluorocyclohexyl)acetamido)pyridin-4-yl)-3,3,3-trifluoropropyl)carbamate (949 mg) as yellow solid. LC/MS: m/z=615.1 [M+H]⁺; rt: 0.55 min (LC/MS-method C).

### Step 7: benzyl ((1S)-2-((4-(1-amino-3,3,3-trifluoropropyl)pyridin-2-yl)amino)-1-(4,4-difluorocyclohexyl)-2-oxoethyl)carbamate

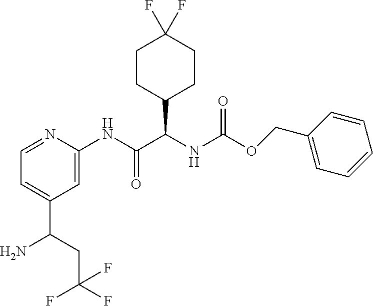 [SMILES: NC(CC(F)(F)F)c1ccnc(NC(=O)[C@H](NC(=O)OCc2ccccc2)C2CCC(F)(F)CC2)c1]

To a solution of tert-butyl (1-(2-((S)-2-(((benzyloxy)carbonyl)amino)-2-(4,4-difluorocyclohexyl)acetamido)pyridin-4-yl)-3,3,3-trifluoropropyl)carbamate (470 mg) in DCM (70 ml) was added TsOH (730 mg) and the mixture stirred for 16 h at RT before 19.5 ml 0.2M aqueous NaOH solution was added and extracted with DCM (20 ml×3). The combined organic phase was dried over Na₂SO₄, filtered and evaporated to afford benzyl ((1S)-2-((4-(1-amino-3,3,3-trifluoropropyl)pyridin-2-yl)amino)-1-(4,4-difluorocyclohexyl)-2-oxoethyl)carbamate (390 mg) that was used directly in the next step without further purification. LC/MS: m/z=515.2 [M+H]⁺; rt: 1.16 min (LC/MS-method B).

### Step 8: benzyl ((1S)-1-(4,4-difluorocyclohexyl)-2-oxo-2-((4-(3,3,3-trifluoro-1-(4,4,4-trifluorobutanamido)propyl)pyridin-2-yl)amino)ethyl)carbamate

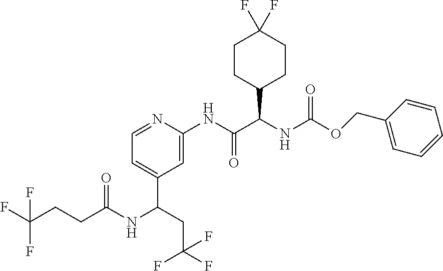 [SMILES: O=C(CCC(F)(F)F)NC(CC(F)(F)F)c1ccnc(NC(=O)[C@H](NC(=O)OCc2ccccc2)C2CCC(F)(F)CC2)c1]

To a solution of 4,4,4-trifluorobutanoic acid (58 mg) in DMF (3 ml) was added HATU (211 mg), DIPEA (0.193 ml) and benzyl ((1S)-2-((4-(1-amino-3,3,3-trifluoropropyl)pyridin-2-yl)amino)-1-(4,4-difluorocyclohexyl)-2-oxoethyl)carbamate (190 mg) dissolved in DMF (2 ml) and the mixture stirred for 16 h at RT. Then additional 4,4,4-trifluorobutanoic acid (58 mg) in DMF (2 ml) with HATU (211 mg) and DIPEA (0.193 ml) were stirred for 20 minutes before addition to the reaction mixture. After stirring for 8 h at RT, the reaction mixture was concentrated under reduced pressure and the residue was dissolved in EA (20 ml) and washed with water (10 ml), NaHCO₃ (10 ml, aq. sat.) and brine (10 ml). The organic phase was concentrated under reduced pressure and residue triturated in diisopropyl ether (10 ml), decanted and dried under reduced pressure. This afforded benzyl ((1S)-1-(4,4-difluorocyclohexyl)-2-oxo-2-((4-(3,3,3-trifluoro-1-(4,4,4-trifluorobutanamido)propyl)pyridin-2-yl)amino)ethyl)carbamate (220 mg) as a colorless solid. LC/MS: m/z=639.3 [M+H]⁺; rt: 2.47 min (LC/MS-method A).

### Step 9: N-(1-(2-((S)-2-amino-2-(4,4-difluorocyclohexyl)acetamido)pyridin-4-yl)-3,3,3-trifluoropropyl)-4,4,4-trifluorobutanamide

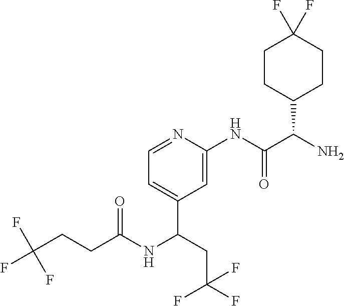 [SMILES: N[C@H](C(=O)Nc1cc(C(CC(F)(F)F)NC(=O)CCC(F)(F)F)ccn1)C1CCC(F)(F)CC1]

Benzyl ((1S)-1-(4,4-difluorocyclohexyl)-2-oxo-2-((4-(3,3,3-trifluoro-1-(4,4,4-trifluorobutanamido)propyl)pyridin-2-yl)amino)ethyl)carbamate (220 mg) was dissolved in MeOH (30 ml) and Pd/C (37 mg) was added and the flask was purged with H₂ from a balloon. After stirring for 6 h under a H₂ atmosphere the catalyst was filtered off and the filtrate was concentrated under reduced pressure to afford N-(1-(2-((S)-2-amino-2-(4,4-difluorocyclohexyl)acetamido)pyridin-4-yl)-3,3,3-trifluoropropyl)-4,4,4-trifluorobutanamide (165 mg). LC/MS: m/z=505.2 [M+H]⁺; rt: 1.38 min (LC/MS-method A).

### Intermediate 58: (S)-2-Amino-2-((1s,4R)-4-methylcyclohexyl)-N-(4-(((S)-2-oxo-4-(trifluoromethyl)imidazolidin-1-yl)methyl)pyridin-2-yl)acetamide

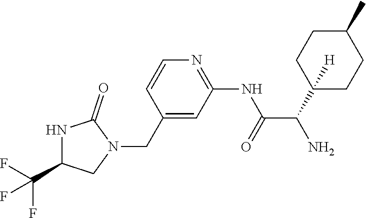

### Step 1: Benzyl ((S)-2-((4-(((tert-butyldimethylsilyl)oxy)methyl)pyridin-2-yl)amino)-1-((1s,4R)-4-methylcyclohexyl)-2-oxoethyl)carbamate

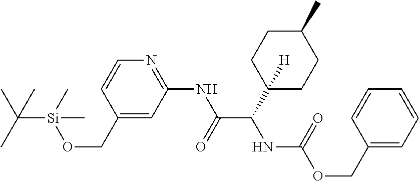

To a solution of 4-(((tert-butyldimethylsilyl)oxy)methyl)pyridin-2-amine (I-001-A1, 5 g) in DCM (50 ml) was added DMAP (5.12 g) and EDCl (8.04 g) at 20° C. and the mixture was stirred for 5 min. Then (2S)-2-(((benzyloxy)carbonyl)amino)-2-(4-methylcyclohexyl)acetic acid (cis/trans mixture, 5 g) was added. The mixture was stirred at 20° C. for 12 h. The reaction mixture was diluted with H₂O (200 ml) and extracted with DCM (100 ml, 2×). The combined organic layers were washed with aqueous NaCl (100 ml), dried over Na₂SO₄, filtered and concentrated under reduced pressure to give a residue. The residue was purified by column chromatography (SiO₂, PPE/EA=1/0 to 4/1) to yield 7.3 g of a diastereomeric mixture. Analytical chiral analysis by SFC (Chiralpak AD-3, 50×4.6 mm I.D., 3 μm; A: CO₂, B: IPA (0.05% DEA), gradient: 5 to 40% B in 3 min, flow: 3.0 ml/min; T: 35° C.): P1: rt: 1.81 min (24%); P2: 2.00 min (8%); P3: 2.18 min 51%); P4: 2.56 min (17%).

The mixture was further purified by preparative SFC (column: Daicel Chiralpak AD (250 mm×30 mm, 10 μm); mobile phase: [A: CO₂, B: IPA (0.1% NH₃ in H₂O)]; 40% B isocratic elution mode) and further separated by SFC (column: Daicel Chiralpak AD (250 mm×30 mm, 10 μm); mobile phase: [A: CO₂, B: IPA (0.1% NH₃ in H₂O)]; B: 45%, isocratic elution mode) to yield 2.73 g of the title compound which corresponded to P3 in the analytical method above. Analytical chiral analysis by SFC (Chiralpak AD-3, 50×4.6 mm I.D., 3 um; phase A: CO₂, phase B: IPA (0.05% DEA), gradient: 5 to 40% B in 3 min, flow: 3.0 ml/min; T: 35° C.): rt: 2.19 min (>99%).

### Step 2: Benzyl ((S)-2-((4-(hydroxymethyl)pyridin-2-yl)amino)-1-((1s,4R)-4-methylcyclohexyl)-2-oxoethyl)carbamate

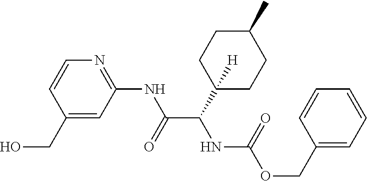

To a solution of benzyl ((S)-2-((4-(((tert-butyldimethylsilyl)oxy)methyl)pyridin-2-yl)amino)-1-((1s,4R)-4-methylcyclohexyl)-2-oxoethyl)carbamate (2.70 g) in THF (27 ml) was added TBAF (1 M in THF, 5.39 ml) at 0° C. The mixture was stirred at 20° C. for 12 h. Then the reaction mixture was diluted with H₂O (100 ml) and extracted with EA (30 ml, 3×). The combined organic layers were washed with aqueous NaCl (30 ml), dried over Na₂SO₄, filtered and concentrated under reduced pressure to give a residue which was purified by column chromatography (SiO₂, PE/EA=1/0 to 1/2) to yield 1.8 g of the title compound. ¹H NMR (400 MHz, DMSO-d6) δ ppm: 10.39 (s, 1H), 8.23 (d, J=5.1 Hz, 1H), 8.07 (s, 1H), 7.51 (br d, J=8.4 Hz, 1H), 7.40-7.24 (m, 5H), 7.03 (d, J=4.9 Hz, 1H), 5.42 (t, J=5.7 Hz, 1H), 5.02 (s, 2H), 4.51 (d, J=5.6 Hz, 2H), 4.28 (br t, J=8.8 Hz, 1H), 1.83-1.73 (m, 1H), 1.65 (br s, 1H), 1.48-1.25 (m, 8H), 0.90 (d, J=6.9 Hz, 3H).

### Step 3: Benzyl ((S)-2-((4-formylpyridin-2-yl)amino)-1-((1s,4R)-4-methylcyclohexyl)-2-oxoethyl)carbamate

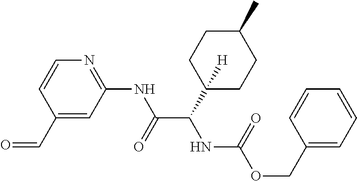

To a solution of benzyl ((S)-2-((4-(hydroxymethyl)pyridin-2-yl)amino)-1-((1s,4R)-4-methylcyclohexyl)-2-oxoethyl)carbamate (1.79 g) in DCM (18 ml) was added DMP (2.77 g). The mixture was stirred at 20° C. for 2 h. Then the reaction mixture was diluted with H₂O (50 ml) and extracted with DCM (20 ml, 3×). The combined organic layers were washed with aqueous NaCl (30 ml), dried over Na₂SO₄, filtered and concentrated under reduced pressure to give a residue which was purified by column chromatography (SiO₂, PE/EA=1/0 to 1/1) to yield 1.4 g of the title compound. Analytical SFC data: (Chiralcel OJ-3, 50 mm×4.6 mm, 3 μm; A: CO₂, B: EtOH (0.05% DEA)]; B %: 5%-40%, flow 3 ml/min, T: 35° C.): RT=1.03 min.

### Step 4: Benzyl ((S)-2-((4-((((S)-2-amino-3,3,3-trifluoropropyl)amino)methyl)pyridin-2-yl)amino)-1-((1s,4R)-4-methylcyclohexyl)-2-oxoethyl)carbamate

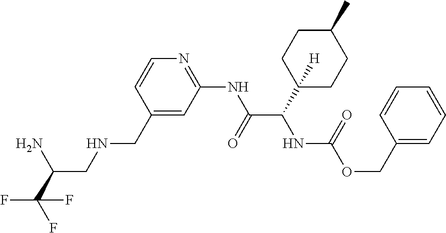

Benzyl ((S)-2-((4-formylpyridin-2-yl)amino)-1-((1s,4R)-4-methylcyclohexyl)-2-oxoethyl)carbamate (400 mg) and (S)-3,3,3-trifluoropropane-1,2-diamine dihydrochloride (Int. 1, Step B6, 236 mg) were dissolved in DCM (13 ml) under Ar. After the addition of TEA (599 μl) the mixture was stirred for 10 min. Then the mixture was heated to 40° C. for 1 h. Then NaBH₃CN (215 mg), dry MeOH (8 ml) and AcOH (291 μl) were added at RT and the mixture was stirred for 2 h. Then the mixture was concentrated in vacuo and DCM and sat. NaHCO₃ solution were added to the residue. The organic phase was separated and the aqueous phase was extracted with DCM (3×). The combined organic phases were dried over Na₂SO₄, filtered and concentrated in vacuo. The residue was further purified by preparative RP HPLC (flow: 25 ml/min; gradient: 95% H₂O+0.05% TFA/5% ACN to 10% H₂O+0.05% TFA/90% ACN; column: Purosphere® STAR-RP18, 25 mm×250 mm, 10 μm). The pure product containing fractions were combined, the ACN was partly removed and the TFA neutralised by concentrated NaHCO₃ solution. After extracting the product with DCM (2×) the combined organic phases were dried over sodium sulphate, filtered and concentrated in vacuo. The residue was dissolved in ACN/H₂O and freeze dried to yield 375 mg of the title compound. LC/MS: m/z=522.2 [M+H]⁺; rt: 1.72 min (LC/MS-method A).

### Step 5: Benzyl ((S)-1-((1s,4R)-4-methylcyclohexyl)-2-oxo-2-((4-(((S)-2-oxo-4-(trifluoromethyl)imidazolidin-1-yl)methyl)pyridin-2-yl)amino)ethyl)carbamate

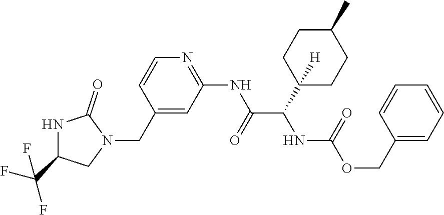

Benzyl ((S)-2-((4-((((S)-2-amino-3,3,3-trifluoropropyl)amino)methyl)pyridin-2-yl)amino)-1-((1s,4R)-4-methylcyclohexyl)-2-oxoethyl)carbamate (373 mg) was dissolved in THE (20 ml) and heated to 65° C. with stirring. After 5 min CDI (348 mg) was added and stirring at 65° C. was continued for 3 h. After cooling the mixture was poured into sat. NaHCO₃ solution and the aqueous phase was extracted with DCM (2×). The combined organic phases were dried over Na₂SO₄, filtered and concentrated in vacuo. The residue was purified by silica gel chromatography (silica gel 12 g, 100% DCM for 5 min, to 90% DCM/10% EtOH in 45 min, 10% EtOH for 10 min). Product containing pure fractions were combined and concentrated in vacuo to yield 369 mg of the title compound. LC/MS: m/z=548.2 [M+H]⁺; rt: 2.35 min (LC/MS-method A).

### Step 6: (S)-2-Amino-2-((1s,4R)-4-methylcyclohexyl)-N-(4-(((S)-2-oxo-4-(trifluoromethyl)-imidazolidin-1-yl)methyl)pyridin-2-yl)acetamide

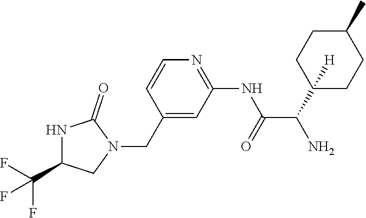

Benzyl ((S)-1-((1s,4R)-4-methylcyclohexyl)-2-oxo-2-((4-(((S)-2-oxo-4-(trifluoromethyl)imidazolidin-1-yl)methyl)pyridin-2-yl)amino)ethyl)carbamate (368 mg) was dissolved in MeOH (8 ml) under Ar atmosphere. Pd/C (36 mg, 10%) was added and the flask was purged with H₂ from a balloon. After stirring for 3 h under a H₂ atmosphere the catalyst was filtered off and washed with MeOH. The filtrate was concentrated to yield 268 mg of the crude title compound. LC/MS: m/z=414.2 [M+H]⁺; rt: 1.24 min (LC/MS-method A).

### Intermediate 59: (S)-2-mino-N-(4-((S)-2-methoxy-1-((S)-2-oxo-4-(trifluoromethyl)-imidazolidin-1-yl)ethyl)pyridin-2-yl)-3-((1,1,1-trifluoro-2-methylpropan-2-yl)oxy)propanamide hydrochloride

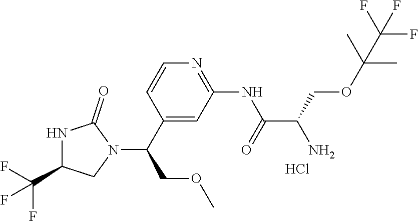 [SMILES: COC[C@H](c1ccnc(NC(=O)[C@@H](N)COC(C)(C)C(F)(F)F)c1)N1C[C@@H](C(F)(F)F)NC1=O ; Cl]

### Step 1: Tert-butyl ((S)-1-((4-((S)-2-methoxy-1-((S)-2-oxo-4-(trifluoromethyl)imidazolidin-1-yl)ethyl)pyridin-2-yl)amino)-1-oxo-3-((1,1,1-trifluoro-2-methylpropan-2-yl)oxy)propan-2-yl)carbamate

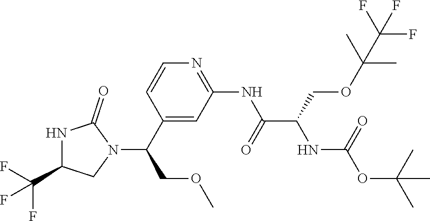 [SMILES: COC[C@H](c1ccnc(NC(=O)[C@H](COC(C)(C)C(F)(F)F)NC(=O)OC(C)(C)C)c1)N1C[C@@H](C(F)(F)F)NC1=O]

To N-(tert-butoxycarbonyl)-O-(1,1,1-trifluoro-2-methylpropan-2-yl)-L-serine (500 mg) was added DCM (7 ml), DMAP (387 mg) and EDCl (608 mg) with stirring. After 5 min (S)-1-((S)-1-(2-aminopyridin-4-yl)-2-methoxyethyl)-4-(trifluoromethyl)imidazolidin-2-one hydrochloride (Int. 54-B-P1; 483 mg) was added and stirring was continued for 1 h. After standing overnight the mixture was poured onto saturated NaHCO₃ solution. The aqueous phase was extracted with DCM (2×). The combined organic phases were dried with magnesium sulphate, filtered and concentrated in vacuo. The residue was purified by silica gel chromatography (silica gel 24 g, DCM/EtOH: 100:0 for 5 min, 100:0 to 92:8 in 30 min, 92:8 for 10 min). Product containing fractions were combined and concentrated in vacuo. The residue was dissolved in ACN/water and freeze dried to yield 613 mg of a mixture of diastereomers which was further purified by preparative SFC (Chiralpak IE 67; 250×30 mm, 5 μm; A: CO₂, B: EtOH, 20% B isocratic mode; flow 150 ml/min) to yield 208 mg of the title compound and 232 mg of tert-butyl ((R)-1-((4-((S)-2-methoxy-1-((S)-2-oxo-4-(trifluoromethyl)imidazolidin-1-yl)ethyl)pyridin-2-yl)amino)-1-oxo-3-((1,1,1-trifluoro-2-methylpropan-2-yl)oxy)propan-2-yl)carbamate. Analytical SFC data: (Chiralpak IE, 150 mm×3 mm, 3 μm; A: CO₂, B: MeOH; B %: 20%, flow 0.8 ml/min, T: 30° C.): rt=8.15 min; diastereomer: 4.47 min. LC/MS: m/z=602.2 [M+H]⁺; rt: 2.22 min (LC/MS-method A).

### Step 2: (S)-2-amino-N-(4-((S)-2-methoxy-1-((S)-2-oxo-4-(trifluoromethyl)imidazolidin-1-yl)ethyl)pyridin-2-yl)-3-((1,1,1-trifluoro-2-methylpropan-2-yl)oxy)propanamide hydrochloride

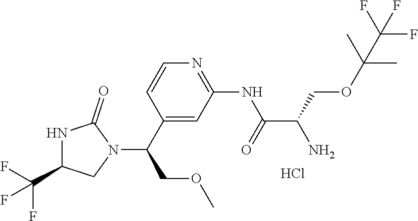 [SMILES: COC[C@H](c1ccnc(NC(=O)[C@@H](N)COC(C)(C)C(F)(F)F)c1)N1C[C@@H](C(F)(F)F)NC1=O ; Cl]

Tert-butyl ((S)-1-((4-((S)-2-methoxy-1-((S)-2-oxo-4-(trifluoromethyl)imidazolidin-1-yl)ethyl)pyridin-2-yl)amino)-1-oxo-3-((1,1,1-trifluoro-2-methylpropan-2-yl)oxy)propan-2-yl)carbamate (207 mg) was dissolved in dioxane (3.5 ml) and HCl in dioxane (4 M, 3.5 ml) was added. After stirring for 2 h the mixture was concentrated in vacuo. The residue was dissolved in a mixture of ACN/water and freeze dried to yield 202 mg of the crude title compound. LC/MS: m/z=502.2 [M+H]⁺; rt: 1.20 min (LC/MS-method A).

### Intermediate 60: (S)-2-Amino-N-(4-(((S)-2-oxo-4-(trifluoromethyl)imidazolidin-1-yl)methyl)pyridin-2-yl)-3-((1,1,1-trifluoro-2-methylpropan-2-yl)oxy)propanamide hydrochloride

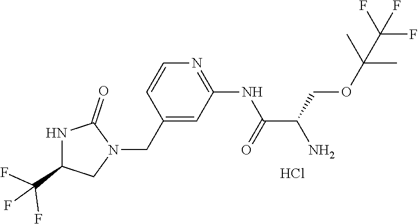 [SMILES: CC(C)(OC[C@H](N)C(=O)Nc1cc(CN2C[C@@H](C(F)(F)F)NC2=O)ccn1)C(F)(F)F ; Cl]

### Step 1: Tert-butyl ((S)-1-oxo-1-((4-(((S)-2-oxo-4-(trifluoromethyl)imidazolidin-1-yl)methyl)pyridin-2-yl)amino)-3-((1,1,1-trifluoro-2-methylpropan-2-yl)oxy)propan-2-yl)carbamate

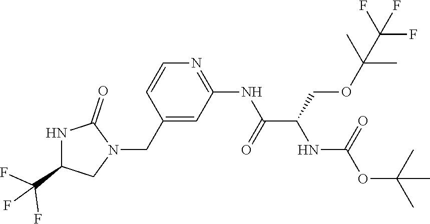 [SMILES: CC(C)(C)OC(=O)N[C@@H](COC(C)(C)C(F)(F)F)C(=O)Nc1cc(CN2C[C@@H](C(F)(F)F)NC2=O)ccn1]

To N-(tert-butoxycarbonyl)-O-(1,1,1-trifluoro-2-methylpropan-2-yl)-L-serine (121 mg) was added DCM (2.5 ml), DMAP (94 mg) and EDCl (147 mg) with stirring. After 5 min (S)-1-((2-aminopyridin-4-yl)methyl)-4-(trifluoromethyl)imidazolidin-2-one (Int. 15; 100 mg) was added and stirring was continued for 3 h. Then the mixture was poured onto saturated NaHCO₃-solution. The aqueous phase was extracted with DCM (2×). The combined organic phases were dried with magnesium sulphate, filtered and concentrated in vacuo. The residue was purified by silica gel chromatography (silica gel 12 g, DCM/EtOH: 100:0 for 5 min, 100:0 to 92:8 in 30 min, 92:8 for 10 min). Product containing fractions were combined and concentrated in vacuo. The residue was dissolved in ACN/water and freeze dried to yield 99 mg of a mixture of diastereomers which was further purified by preparative SFC (Chiralpak IE 67; 250×30 mm, 5 μm; A: CO₂, B: EtOH, 20% B isocratic mode; flow 150 ml/min) to yield 34 mg of the title compound and 38 mg of tert-butyl ((R)-1-oxo-1-((4-(((S)-2-oxo-4-(trifluoromethyl)imidazolidin-1-yl)methyl)pyridin-2-yl)amino)-3-((1,1,1-trifluoro-2-methylpropan-2-yl)oxy)propan-2-yl)carbamate. Analytical SFC data: (Chiralpak IE/190, 150 mm×3 mm, 3 μm; A: CO₂, B: MeOH; B %: 15%, flow 0.8 ml/min, T: 30° C.): rt=7.12 min; diastereomer: 4.83 min. LC/MS: m/z=558.1 [M+H]⁺; rt: 2.17 min (LC/MS-method A).

### Step 2: (S)-2-Amino-N-(4-(((S)-2-oxo-4-(trifluoromethyl)imidazolidin-1-yl)methyl)pyridin-2-yl)-3-((1,1,1-trifluoro-2-methylpropan-2-yl)oxy)propanamide hydrochloride

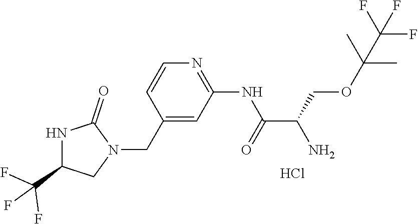 [SMILES: CC(C)(OC[C@H](N)C(=O)Nc1cc(CN2C[C@@H](C(F)(F)F)NC2=O)ccn1)C(F)(F)F ; Cl]

Tert-butyl ((S)-1-oxo-1-((4-(((S)-2-oxo-4-(trifluoromethyl)imidazolidin-1-yl)methyl)pyridin-2-yl)amino)-3-((1,1,1-trifluoro-2-methylpropan-2-yl)oxy)propan-2-yl)carbamate (32 mg) was dissolved in dioxane (1 ml) and HCl in dioxane (4 M, 1 ml) was added. After stirring for 2.5 h the mixture was concentrated in vacuo. The residue was dissolved in a mixture of ACN/water and freeze dried to yield 30 mg of the crude title compound. LC/MS: m/z=458.2 [M+H]⁺; rt: 1.15 min (LC/MS-method A).

### Intermediate 61: (S)-2-amino-2-(4,4-difluorocyclohexyl)-N-(4-((R)-1-((S)-2-oxo-4-(trifluoromethyl)imidazolidin-1-yl)butyl)pyridin-2-yl)acetamide

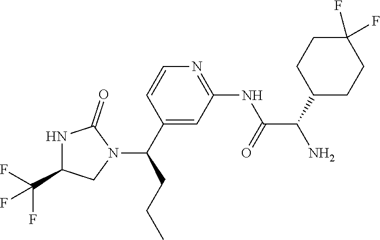 [SMILES: CCC[C@H](c1ccnc(NC(=O)[C@@H](N)C2CCC(F)(F)CC2)c1)N1C[C@@H](C(F)(F)F)NC1=O]

### Step 1: tert-butyl ((1S)-1-(4,4-difluorocyclohexyl)-2-((4-(1-hydroxybutyl)pyridin-2-yl)amino)-2-oxoethyl)carbamate

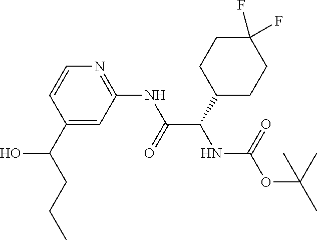 [SMILES: CCCC(O)c1ccnc(NC(=O)[C@@H](NC(=O)OC(C)(C)C)C2CCC(F)(F)CC2)c1]

To a solution of tert-butyl (S)-(1-(4,4-difluorocyclohexyl)-2-((4-formylpyridin-2-yl)amino)-2-oxoethyl)carbamate (Int. 3, step 3; 3 g) in THE was added propylmagnesium bromide solution (15.1 ml, 2M) at 0° C. and the mixture was stirred at RT for 20 min. and then NH₄Cl solution (aq., sat.) at 0° C. was added. The mixture was diluted with water (200 ml) and extracted with EA:DCM (100 ml×2, 9:1) and the combined organic layers were washed with brine (100 ml×2), dried over Na₂SO₄, filtered and concentrated under reduced pressure. The residue was purified by FC (SiO₂, EA (20% DCM)/PE 0:1 to 9:1 to afford tert-butyl ((1S)-1-(4,4-difluorocyclohexyl)-2-((4-(1-hydroxybutyl)pyridin-2-yl)amino)-2-oxoethyl)carbamate (1.49 g) as a light-yellow solid. LC/MS: m/z=442.2 [M+H]⁺; rt: 0.39 min (LC/MS-method C).

### Step 2: tert-butyl (S)-(2-((4-butyrylpyridin-2-yl)amino)-1-(4,4-difluorocyclohexyl)-2-oxoethyl)carbamate

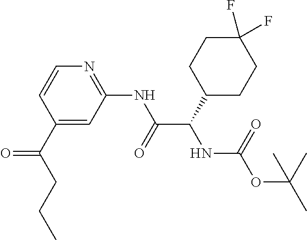 [SMILES: CCCC(=O)c1ccnc(NC(=O)[C@@H](NC(=O)OC(C)(C)C)C2CCC(F)(F)CC2)c1]

Was prepared by the procedure described in Int. 48, Step 2 using tert-butyl ((1S)-1-(4,4-difluorocyclohexyl)-2-((4-(1-hydroxybutyl)pyridin-2-yl)amino)-2-oxoethyl)carbamate (Int. 61, Step 1; 1.49 g) to afford tert-butyl (S)-(2-((4-butyrylpyridin-2-yl)amino)-1-(4,4-difluorocyclohexyl)-2-oxoethyl)carbamate (1.39 g) as a colorless solid. LC/MS: m/z=440.2 [M+H]⁺; rt: 0.49 min (LC/MS-method C).

### Step 3A and 3B: tert-butyl ((S)-1-(4,4-difluorocyclohexyl)-2-oxo-2-((4-((R)-1-((S)-2-oxo-4-(trifluoromethyl)imidazolidin-1-yl)butyl)pyridin-2-yl)amino)ethyl)carbamate and tert-butyl ((S)-1-(4,4-difluorocyclohexyl)-2-oxo-2-((4-((S)-1-((S)-2-oxo-4-(trifluoromethyl)imidazolidin-1-yl)butyl)pyridin-2-yl)amino)ethyl)carbamate

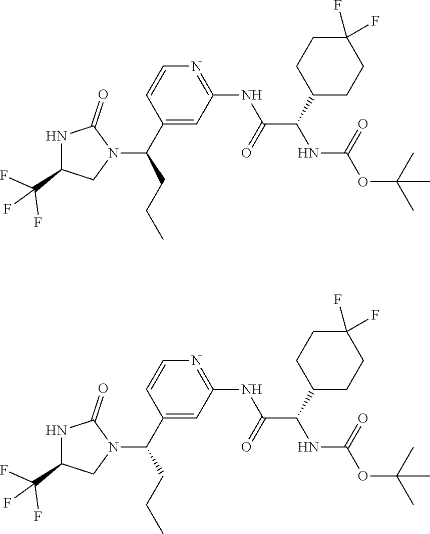 [SMILES: CCC[C@H](c1ccnc(NC(=O)[C@@H](NC(=O)OC(C)(C)C)C2CCC(F)(F)CC2)c1)N1C[C@@H](C(F)(F)F)NC1=O ; CCC[C@@H](c1ccnc(NC(=O)[C@@H](NC(=O)OC(C)(C)C)C2CCC(F)(F)CC2)c1)N1C[C@@H](C(F)(F)F)NC1=O]

Following the procedure described in Int. 50, Step 4 and Int. 50, Step 5 using tert-butyl (S)-(2-((4-butyrylpyridin-2-yl)amino)-1-(4,4-difluorocyclohexyl)-2-oxoethyl)-carbamate (1.39 g) crude product (1.88 g) was obtained. The two diastereomers were separated by FC (SiO₂, Eluent of 0~30~50~70% Ethyl acetate (10% DCM+0.3% NH₃H₂O)/Petroleum ether gradient @80 mL/min) to afford tert-butyl ((S)-1-(4,4-difluorocyclohexyl)-2-oxo-2-((4-((R)-1-((S)-2-oxo-4-(trifluoromethyl)imidazolidin-1-yl)butyl)pyridin-2-yl)amino)ethyl)carbamate (Step 3A; 733 mg) as a white foam; LC/MS: m/z=578.3 [M+H]⁺; rt: 0.45 min (LC/MS-method C); and tert-butyl ((S)-1-(4,4-difluorocyclohexyl)-2-oxo-2-((4-((S)-1-((S)-2-oxo-4-(trifluoromethyl)imidazolidin-1-yl)butyl)pyridin-2-yl)amino)ethyl)carbamate (Step 3B; 772 mg) as a white foam; LC/MS: m/z=578.5 [M+H]⁺; rt: 0.45 min (LC/MS-method C).

### Step 4: (S)-2-amino-2-(4,4-difluorocyclohexyl)-N-(4-((R)-1-((S)-2-oxo-4-(trifluoromethyl)-imidazolidin-1-yl)butyl)pyridin-2-yl)acetamide

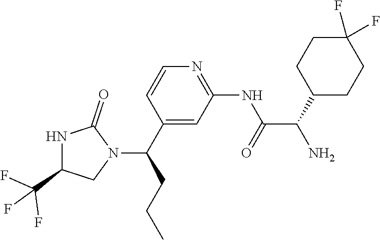 [SMILES: CCC[C@H](c1ccnc(NC(=O)[C@@H](N)C2CCC(F)(F)CC2)c1)N1C[C@@H](C(F)(F)F)NC1=O]

Following the procedure described in Int. 57, Step 7 using tert-butyl ((S)-1-(4,4-difluorocyclohexyl)-2-oxo-2-((4-((R)-1-((S)-2-oxo-4-(trifluoromethyl)imidazolidin-1-yl)butyl)pyridin-2-yl)amino)ethyl)carbamate (Step 3A; 360 mg) afforded (S)-2-amino-2-(4,4-difluorocyclohexyl)-N-(4-((R)-1-((S)-2-oxo-4-(trifluoromethyl)imidazolidin-1-yl)butyl)pyridin-2-yl)acetamide (300 mg). LC/MS: m/z=478.2 [M+H]⁺; rt: 1.42 min (LC/MS-method A).

### Intermediate 62-DS1, -DS2, DS3, -DS4: 2-Amino-2-(4-fluorocyclohexyl)-N-(4-((S)-2-methoxy-1-((S)-2-oxo-4-(trifluoromethyl)imidazolidin-1-yl)ethyl)pyridin-2-yl)acetamide

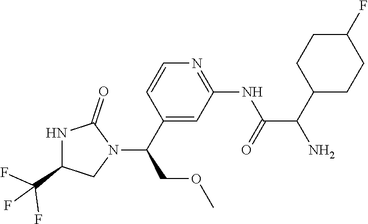 [SMILES: COC[C@H](c1ccnc(NC(=O)C(N)C2CCC(F)CC2)c1)N1C[C@@H](C(F)(F)F)NC1=O]

### Step 1: 1,4-Dioxaspiro[4.5]decane-8-carboxylic acid

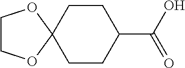 [SMILES: O=C(O)C1CCC2(CC1)OCCO2]

To a mixture of ethyl 1,4-dioxaspiro[4.5]decane-8-carboxylate (50 g) in THE (500 ml) and H₂O (250 ml) was added NaOH (14.00 g) and MeOH (140 ml) in one portion under N₂. The mixture was stirred at 20° C. for 12 h. Then the mixture was concentrated, diluted with water (4 ml) and adjusted to pH~5 with aqueous HCl (1 M). Then the aqueous phase was extracted with DCM (15 ml, 3×). The combined organic phase was washed with brine (15 ml), dried over Na₂SO₄, filtered and concentrated to yield 40 g of the title compound. ¹H NMR (400 MHz, CDCl₃) δ ppm: 3.95 (s, 4H), 2.43-2.36 (m, 1H), 2.03-1.93 (m, 2H), 1.90-1.75 (m, 4H), 1.63-1.51 (m, 2H).

### Step 2: 8-Fluoro-1,4-dioxaspiro[4.5]decane

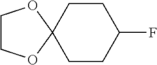 [SMILES: FC1CCC2(CC1)OCCO2]

To a mixture of 1,4-dioxaspiro[4.5]decane-8-carboxylic acid (40 g) and 1-(chloromethyl)-4-fluoro-1,4-diazoniabicyclo[2.2.2]octane; ditetrafluoroborate (160 g) in ACN (600 ml) and H₂O (600 ml) was added 4-methoxy-2-(4-methoxy-2-pyridyl)pyridine (4.65 g), diacetoxyiron (3.74 g) and 2,6-dimethylpyridine (41.43 g) in one portion under N₂. The mixture was stirred at 20° C. for 2 h. Then the reaction mixture was diluted with water (20 ml) and extracted with EA (30 ml, 3×). The combined organic layers were washed with brine (30 ml), dried over Na₂SO₄, filtered and concentrated under reduced pressure. The residue was purified by column chromatography (SiO₂, PE/EA=1/0 to 20:1) to yield 5 g of the title compound. ¹H NMR (400 MHz, CDCl₃) δ ppm: 4.85-4.60 (m, 1H), 4.02-3.86 (m, 4H), 2.02-1.93 (m, 2H), 1.91-1.76 (m, 4H), 1.65-1.56 (m, 2H).

### Step 3: 4-Fluorocyclohexan-1-one

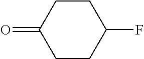

To a mixture of 8-Fluoro-1,4-dioxaspiro[4.5]decane (5.4 g) in THE (54 ml) and H₂O (54 ml) was added aqueous HCl (4 M, 54.00 ml) in one portion under N₂. The mixture was stirred at 40° C. for 4 h. Then the reaction mixture was diluted with water (20 ml). The aqueous phase was extracted with DCM (50 ml, 3×). The combined organic layers were washed with brine (30 ml), dried over Na₂SO₄, filtered and concentrated under reduced pressure to yield 3.9 g of the title compound.

### Step 4: Methyl 2-((tert-butoxycarbonyl)amino)-2-(4-fluorocyclohexylidene)acetate

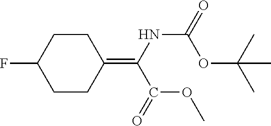 [SMILES: COC(=O)C(NC(=O)OC(C)(C)C)=C1CCC(F)CC1]

To a mixture of methyl 2-(tert-butoxycarbonylamino)-2-dimethoxyphosphoryl-acetate (12.36 g) in NMP (9 ml) was added DBU (5.67 ml) in one portion under N₂. The mixture was stirred at 20° C. for 30 min, then 4-fluorocyclohexan-1-one (4.6 g) dissolved in NMP (9 ml) was added and the mixture was stirred at 20° C. for 3 h. Then the mixture was diluted with water (4 ml), the pH was adjusted to ~6 with aqueous HCl (1 M) and after filtration the filtrate was concentrated in vacuo to yield 6 g of the title compound. ¹H NMR (400 MHz, CDCl₃) δ ppm: 5.98-5.51 (m, 1H), 4.97-4.71 (m, 1H), 3.77 (s, 3H), 2.96-2.65 (m, 2H), 2.55-2.30 (m, 2H), 2.08-1.78 (m, 4H), 1.46 (s, 9H).

### Step 5: Methyl 2-((tert-butoxycarbonyl)amino)-2-(4-fluorocyclohexyl)acetate

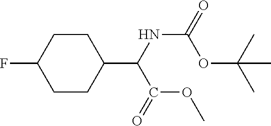 [SMILES: COC(=O)C(NC(=O)OC(C)(C)C)C1CCC(F)CC1]

To a mixture of methyl 2-((tert-butoxycarbonyl)amino)-2-(4-fluorocyclohexylidene)acetate (6 g) in MeOH (60 ml) was added Pd/C (2.22 g, 10%) in one portion under N₂. The mixture was stirred at 20° C. under H₂ for 2 h. Then the reaction mixture was filtered and concentrated under reduced pressure. The crude product was used directly in the next step without further purification to yield 6 g of the title compound.

### Step 6: 2-((Tert-butoxycarbonyl)amino)-2-(4-fluorocyclohexyl)acetic acid

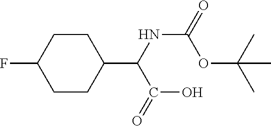 [SMILES: CC(C)(C)OC(=O)NC(C(=O)O)C1CCC(F)CC1]

To a mixture of methyl 2-((tert-butoxycarbonyl)amino)-2-(4-fluorocyclohexyl)acetate (6 g) in MeOH (60 ml) and H₂O (60 ml) was added LiOH (993 mg) in one portion under N₂. The mixture was stirred at 20° C. for 2 h. Then the reaction mixture was diluted with water (20 ml) and the pH of the mixture was adjusted ~5 with aqueous HCl. The aqueous phase was extracted with EA (30 ml, 3×) and the combined organic layers were washed with brine (30 ml), dried over Na₂SO₄, filtered and concentrated under reduced pressure to yield 5.7 g of the title compound.

### Step 7: 2-Amino-2-(4-fluorocyclohexyl)acetic acid hydrochloride

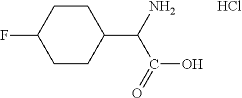 [SMILES: Cl ; NC(C(=O)O)C1CCC(F)CC1]

To 2-((tert-butoxycarbonyl)amino)-2-(4-fluorocyclohexyl)acetic acid (5.7 g) was added HCl/dioxane (4 M, 5.18 ml) in one portion under N₂. The mixture was stirred at 20° C. for 2 h. Then the reaction mixture was concentrated under reduced pressure to yield 4.38 g of the title compound.

### Step 8: 2-(((Benzyloxy)carbonyl)amino)-2-(4-fluorocyclohexyl)acetic acid

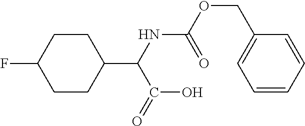 [SMILES: O=C(NC(C(=O)O)C1CCC(F)CC1)OCc1ccccc1]

To 2-amino-2-(4-fluorocyclohexyl)acetic acid hydrochloride (4.38 g) in THE (45 ml) was added a solution of NaOH (3.31 g) in H₂O (45 ml) under N₂. The mixture was stirred at 20° C. for 30 min, then CbzCl (5.30 g) was added at 0° C. and the mixture was stirred at 20° C. for 2 h. Then the reaction mixture was diluted with water (20 ml) and the pH was adjusted to ~5 with aqueous HCl solution. The aqueous phase was extracted with EA (30 ml, 3×). The combined organic layers were washed with brine (30 ml), dried over Na₂SO₄, filtered and concentrated under reduced pressure. The residue was triturated with PE/EA=20:1 (100 ml) and filtered to yield 5 g of the title compound. LC/MS: m/z=310.1 [M+H]⁺; rt: 0.39 min (LC/MS-method C).

### Step 9-P1, -P2, -P3, -P4: Benzyl ((S)-1-((1r,4S)-4-fluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)carbamate, benzyl ((R)-1-((1r,4R)-4-fluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)carbamate, benzyl ((R)-1-((1s,4S)-4-fluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)carbamate, and benzyl ((S)-1-((1s,4R)-4-fluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)carbamate

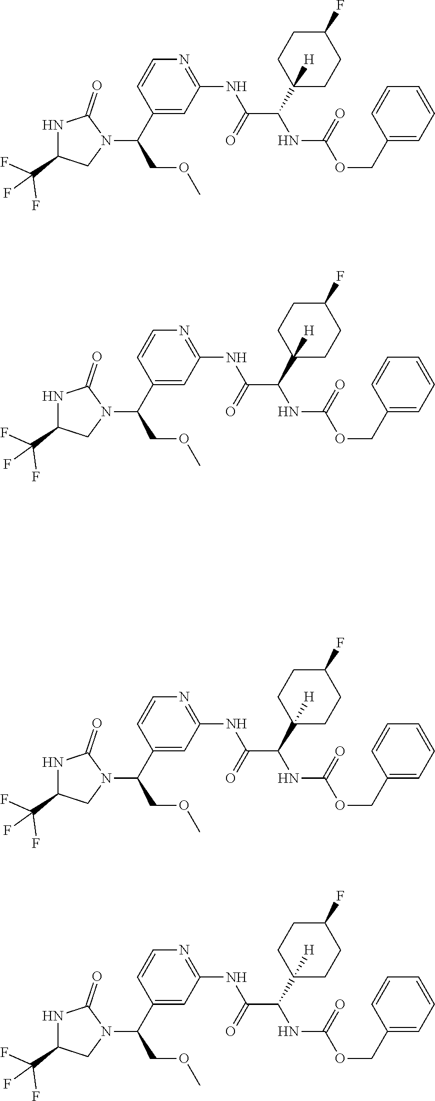

To 2-(((benzyloxy)carbonyl)amino)-2-(4-fluorocyclohexyl)acetic acid (2.95 g) and (S)-1-((S)-1-(2-aminopyridin-4-yl)-2-methoxyethyl)-4-(trifluoromethyl)imidazolidin-2-one hydrochloride (Int. 54-B-P1, 2.95 g) in pyridine (36 ml) was added EDCl (5.58 g) in one portion under N₂. The mixture was stirred at 20° C. for 2 h. Then the reaction mixture was diluted with water (20 ml) and extracted with EA (30 ml, 3×). The combined organic layers were washed with brine (30 ml), dried over Na₂SO₄, filtered and concentrated under reduced pressure. The residue was purified by prep-HPLC (column: Phenomenex luna C18 (250×70 mm, 10 μm); mobile phase: [A: water (10% NH₄HCO₃); B: ACN]; gradient: 30%-60% B over 25 min; flow 140 ml/min) to yield 2.7 g of a diastereomeric mixture which was further separated by preparative SFC (column: Daicel Chiralpak AD (250 mm×30 mm, 10 μm); mobile phase: [A: CO₂; B: IPA (0.1% NH₃ in H₂O)]; B: 45%, isocratic elution mode). The respective product containing fractions were collected and concentrated in vacuo to yield 536 mg of P1, 494 mg of P2, 533 mg of P3 and 461 mg of P4. Analytical SFC data: (Chiralpak AD-3, 50 mm×4.6 mm, 3 μm; A: CO₂, B: IPA (0.05 & DEA); B: 40%, flow 3 ml/min, T: 35° C.): P1: rt=0.65 min, >99%; P2: 0.78 min, 98%; P3: 0.96 min, 98%; P4: 1.26 min, 98%.

### Step 10: 2-Amino-2-(4-fluorocyclohexyl)-N-(4-((S)-2-methoxy-1-((S)-2-oxo-4-(trifluoromethyl)imidazolidin-1-yl)ethyl)pyridin-2-yl)acetamide DS1, DS2, DS3, DS4

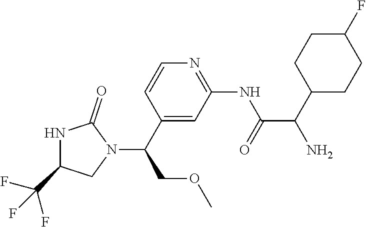 [SMILES: COC[C@H](c1ccnc(NC(=O)C(N)C2CCC(F)CC2)c1)N1C[C@@H](C(F)(F)F)NC1=O]

### Diastereomer 1:

Benzyl (1-(4-fluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)carbamate (Step 9-P1; 250 mg) was dissolved in MeOH (6 ml) under Ar atmosphere. Pd/C (22 mg, 10%) was added and the flask was purged with H₂ from a balloon. After stirring for 3 h under a H₂ atmosphere the catalyst was filtered off and washed with MeOH. The filtrate was concentrated to yield 192 mg of the crude title compound. LC/MS: m/z=462.2 [M+H]⁺; rt: 1.07 min (LC/MS-method A).

The other diastereomers DS2, DS3 and DS4, were analogously prepared to DS1 using the respective diastereomers Step 9-P2, -P3 and -P4. DS2: 193 mg; LC/MS: m/z=462.2 [M+H]⁺; rt: 1.05 min (LC/MS-method A). DS3: 193 mg; LC/MS: m/z=462.2 [M+H]⁺; rt: 1.08 min (LC/MS-method A). DS4: 194 mg; LC/MS: m/z=462.2 [M+H]⁺; rt: 1.06 min (LC/MS-method A).

### Intermediate 63: N-((2-((S)-2-amino-2-(4,4-difluorocyclohexyl)acetamido)pyridin-4-yl)(cyclopropyl)methyl)-3,3,3-trifluoropropanamide

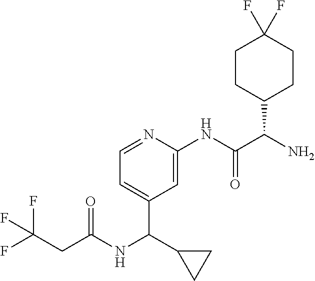 [SMILES: N[C@H](C(=O)Nc1cc(C(NC(=O)CC(F)(F)F)C2CC2)ccn1)C1CCC(F)(F)CC1]

Prepared following the procedure described for Intermediate 41 using 3,3,3-trifluoropropanoic acid to afford N-((2-((S)-2-amino-2-(4,4-difluorocyclohexyl)acetamido)pyridin-4-yl)(cyclopropyl)methyl)-3,3,3-trifluoropropanamide Intermediate 63 (90 mg) that was used directly in the next step. LC/MS: m/z=449.1 [M+H]⁺; rt: 1.22 min (LC/MS-method A).

### Intermediate 64: N—((R)-1-(2-((S)-2-amino-2-(4,4-difluorocyclohexyl)acetamido)pyridin-4-yl)ethyl)-4,4,4-trifluorobutanamide

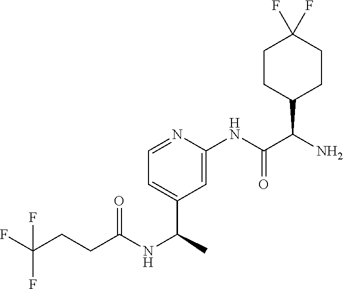 [SMILES: C[C@@H](NC(=O)CCC(F)(F)F)c1ccnc(NC(=O)[C@H](N)C2CCC(F)(F)CC2)c1]

### Step 1: benzyl ((S)-1-(4,4-difluorocyclohexyl)-2-oxo-2-((4-((R)-1-(4,4,4-trifluorobutanamido)ethyl)pyridin-2-yl)amino)ethyl)carbamate

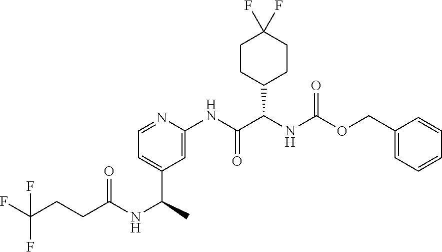 [SMILES: C[C@@H](NC(=O)CCC(F)(F)F)c1ccnc(NC(=O)[C@@H](NC(=O)OCc2ccccc2)C2CCC(F)(F)CC2)c1]

Using the procedure described in Int. 57, Step 8, employing benzyl ((S)-2-((4-((R)-1-aminoethyl)pyridin-2-yl)amino)-1-(4,4-difluorocyclohexyl)-2-oxoethyl)carbamate (Int. 49, Step 5; 190 mg) afforded benzyl ((S)-1-(4,4-difluorocyclohexyl)-2-oxo-2-((4-((R)-1-(4,4,4-trifluorobutanamido)ethyl)pyridin-2-yl)amino)ethyl)carbamate (180 mg) as a colorless solid. LC/MS: m/z=571.3 [M+H]⁺; rt: 1.37 min (LC/MS-method B).

### Step 2: N—((R)-1-(2-((S)-2-amino-2-(4,4-difluorocyclohexyl)acetamido)pyridin-4-yl)ethyl)-4,4,4-trifluorobutanamide

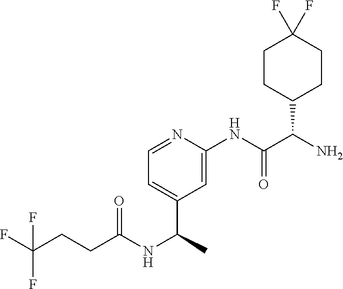 [SMILES: C[C@@H](NC(=O)CCC(F)(F)F)c1ccnc(NC(=O)[C@@H](N)C2CCC(F)(F)CC2)c1]

Was prepared as described for N-(1-(2-((S)-2-amino-2-(4,4-difluorocyclohexyl)acetamido)pyridin-4-yl)-3,3,3-trifluoropropyl)-4,4,4-trifluorobutanamide Int. 57, using benzyl ((S)-1-(4,4-difluorocyclohexyl)-2-oxo-2-((4-((R)-1-(4,4,4-trifluorobutanamido)ethyl)pyridin-2-yl)amino)ethyl)carbamate (Step 1; 180 mg) to afford N—((R)-1-(2-((S)-2-amino-2-(4,4-difluorocyclohexyl)acetamido)pyridin-4-yl)ethyl)-4,4,4-trifluorobutanamide I-063 (140 mg). LC/MS: m/z=437.2 [M+H]⁺; rt: 0.90 min (LC/MS-method B).

### Intermediate 65: (S)-2-amino-2-(4,4-difluorocyclohexyl)-N-(4-(2-((S)-2-oxo-4-(trifluoromethyl)imidazolidin-1-yl)propan-2-yl)pyridin-2-yl)acetamide

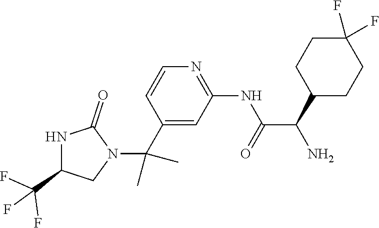 [SMILES: CC(C)(c1ccnc(NC(=O)[C@H](N)C2CCC(F)(F)CC2)c1)N1C[C@@H](C(F)(F)F)NC1=O]

### Step 1: 2-(2-chloropyridin-4-yl)propan-2-amine

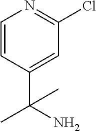

To a solution of compound 2-chloro-4-cyanopyridine (56 g) in THE (896 ml) was added MeMgBr (3 M, 404.17 ml) at 0° C. followed by the addition of Ti(i-PrO)₄ (119.29 ml) at 0° C. after 1 h. The mixture was stirred at 50° C. for 3 hr before addition NaOH (500 ml, 1 M, aq.) at 0° C., then filtered and diluted with water (2 L) and extracted with DCM (800 ml×3). The combined organic layers were washed with brine (1000 ml×2), dried over Na₂SO₄, filtered and concentrated under reduced pressure to afford 2-(2-chloropyridin-4-yl)propan-2-amine (51.7 g) as a brown gum that was used directly in the next step. ¹H NMR (400 MHz, DMSO-d6) δ ppm: 8.30 (d, J=5.3 Hz, 1H), 7.62 (d, J=1.0 Hz, 1H), 7.52 (dd, J=1.6, 5.3 Hz, 1H), 1.34 (s, 6H).

### Step 2: tert-butyl (2-(2-chloropyridin-4-yl)propan-2-yl)carbamate

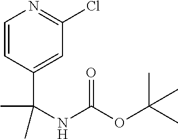 [SMILES: CC(C)(C)OC(=O)NC(C)(C)c1ccnc(Cl)c1]

To a solution of compound 2-(2-chloropyridin-4-yl)propan-2-amine (62 g) in MeOH (620 ml) was added (Boc)₂O (95.16 g) and TEA (101.14 ml) at 0° C. and the mixture was stirred at 20° C. for 12 hr before the reaction mixture was concentrated under reduced pressure. The residue was purified by flash FC (SiO₂, Eluent of 0~30~50% EA/PE gradient @80 mL/min) to afford tert-butyl (2-(2-chloropyridin-4-yl)propan-2-yl)carbamate (13.0 g) as a brown oil. ¹H NMR (400 MHz, CDCl3) δ ppm: 8.32 (d, J=5.3 Hz, 1H), 7.32 (s, 1H), 7.22 (br d, J=5.3 Hz, 1H), 4.99 (br s, 1H), 1.59 (s, 6H), 1.47-1.33 (m, 9H).

### Step 3: tert-butyl allyl(2-(2-chloropyridin-4-yl)propan-2-yl)carbamate

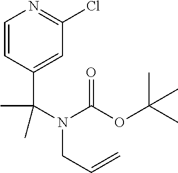 [SMILES: C=CCN(C(=O)OC(C)(C)C)C(C)(C)c1ccnc(Cl)c1]

To a solution of NaH (3.84 g, 60%) in DMF (130 ml) was added tert-butyl (2-(2-chloropyridin-4-yl)propan-2-yl)carbamate (13 g) at 0° C., followed by 3-bromoprop-1-ene (8.71 g) after 30 min. and the mixture was stirred at RT for 1 hr before being diluted with water (600 ml) at 0° C. and extracted with EA (200 ml×3). The combined organic layers were washed with brine (200 ml×3), dried over Na₂SO₄, filtered and concentrated under reduced pressure to give a residue, which was purified by FC (SiO₂, Eluent of 0~10~20%~50% EA/PE) to afford tert-butyl allyl(2-(2-chloropyridin-4-yl)propan-2-yl)carbamate (12 g) as a yellow oil. LC/MS: m/z=311.2 [M+H]⁺; rt: 0.75 min (LC/MS-method C).

### Step 4: tert-butyl (2-(2-chloropyridin-4-yl)propan-2-yl)(2-oxoethyl)carbamate

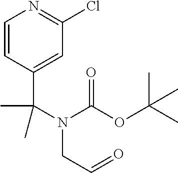 [SMILES: CC(C)(C)OC(=O)N(CC=O)C(C)(C)c1ccnc(Cl)c1]

To a solution of tert-butyl allyl(2-(2-chloropyridin-4-yl)propan-2-yl) (12 g) and potassium osmate (IV) hydrate (2.85) in dioxane (202 ml) and water (202 ml) was added NalO₄ (41.29 g) at 0° C. and the mixture was stirred at RT C for 1 hr. The reaction mixture was diluted with water (400 ml) at 0° C. and extracted with EA (200 ml×3) and the combined organic layers were washed with brine (200 ml×3), dried over Na₂SO₄, filtered and concentrated under reduced pressure to give a residue, which purified was purified by FC (SiO₂, Eluent of 0~10~20%~50%~80% EA/PE). This afforded tert-butyl (2-(2-chloropyridin-4-yl)propan-2-yl)(2-oxoethyl)carbamate I-064-4 (5.3 g) as a yellow oil. LC/MS: m/z=313.1 [M+H]⁺; rt: 0.39 min (LC/MS-method C).

### Step 5: tert-butyl (R)-(2-((tert-butylsulfinyl)imino)ethyl)(2-(2-chloropyridin-4-yl)propan-2-yl)carbamate

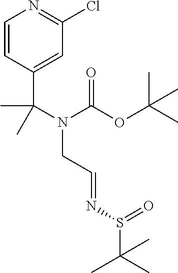 [SMILES: CC(C)(C)OC(=O)N(C/C=N/[S@](=O)C(C)(C)C)C(C)(C)c1ccnc(Cl)c1]

To a solution of tert-butyl (2-(2-chloropyridin-4-yl)propan-2-yl)(2-oxoethyl)carbamate (5.3 g) and (R)-2-methylpropane-2-sulfinamide (2.46 g) in DCM (100 ml) was added pyridinium p-toluenesulfonate (212.91 mg) and MgSO₄ (10.20 g) at 0° C. and the mixture was stirred at RT for 12 hr before the reaction mixture was filtered and concentrated under reduced pressure. The residue was purified by FC (SiO₂ Eluent of 0~10~20%~50%~80% EA/PE) to afford tert-butyl (R)-(2-((tert-butylsulfinyl)imino)ethyl)(2-(2-chloropyridin-4-yl)propan-2-yl)carbamate (7 g) as a yellow oil. LC/MS: m/z=438.1 [M+H]⁺; rt: 0.46 min (LC/MS-method C).

### Step 6: tert-butyl ((S)-2-(((R)-tert-butylsulfinyl)amino)-3,3,3-trifluoropropyl)(2-(2-chloropyridin-4-yl)propan-2-yl)carbamate

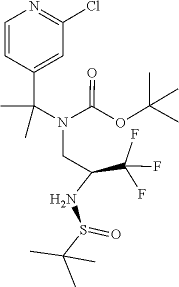

To a solution of tert-butyl (R)-(2-((tert-butylsulfinyl)imino)ethyl)(2-(2-chloropyridin-4-yl)propan-2-yl)carbamate (7 g) and tetrabutylammonium acetate (7.61 g) in THE (150 ml) was added TMS-CF₃ (7.18 g) at −55° C. and stirred at −55° C. for 1 hr before the reaction was allowed to warm to 0° C. over 1 hr. The reaction mixture was concentrated under reduced and the residue was diluted with water (500 ml) and extracted with EA (200 ml×3) before the combined organic layers were washed with brine (300 ml×2), dried over Na₂SO₄, filtered and concentrated under reduced pressure. The residue was purified by FC (SiO₂, PE/EA=1:0 to 2:1 to 1:1) affording tert-butyl ((S)-2-(((R)-tert-butylsulfinyl)amino)-3,3,3-trifluoropropyl)(2-(2-chloropyridin-4-yl)propan-2-yl)carbamate (2.6 g) as a yellow oil and remaining starting material (3 g). LC/MS: m/z=486.0 [M+H]⁺; rt: 0.50 min (LC/MS-method C).

### Step 7: (S)—N1-(2-(2-chloropyridin-4-yl)propan-2-yl)-3,3,3-trifluoropropane-1,2-diamine

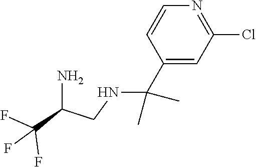 [SMILES: CC(C)(NC[C@H](N)C(F)(F)F)c1ccnc(Cl)c1]

To a solution of tert-butyl ((S)-2-(((R)-tert-butylsulfinyl)amino)-3,3,3-trifluoropropyl)(2-(2-chloropyridin-4-yl)propan-2-yl)carbamate (3.84 g) in MeOH (30 ml) was added HCl/dioxane (2 M, 59.26 mL) and the mixture was stirred at RT for 2 hr before the temperature was increased to 50° C. for 2 hr. The reaction mixture was concentrated under reduced pressure and the residue diluted with DCM (100 ml) and Na₂CO₃ solution (aq., sat.) was added at 0° C. until neutral pH and then extracted with DCM (100 ml×2). The combined organic layers were washed with brine (100 ml×2), dried over Na₂SO₄, filtered and concentrated under reduced pressure to afford: (S)—N1-(2-(2-chloropyridin-4-yl)propan-2-yl)-3,3,3-trifluoropropane-1,2-diamine (2.12 g) as a yellow oil that was used without further purification. LC/MS: m/z=282.0 [M+H]⁺; rt: 0.22 min (LC/MS-method C).

### Step 8: (S)-1-(2-(2-chloropyridin-4-yl)propan-2-yl)-4-(trifluoromethyl)imidazolidin-2-one

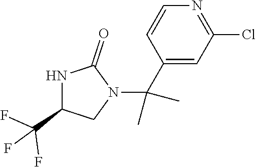 [SMILES: CC(C)(c1ccnc(Cl)c1)N1C[C@@H](C(F)(F)F)NC1=O]

Following the procedure described for Int. 54 B-5-P1 and -P2, using (S)—N1-(2-(2-chloropyridin-4-yl)propan-2-yl)-3,3,3-trifluoropropane-1,2-diamine (2.12 g), (S)-1-(2-(2-chloropyridin-4-yl)propan-2-yl)-4-(trifluoromethyl)imidazolidin-2-one (2.04 g) was obtained as a colorless oil. LC/MS: m/z=308.0 [M+H]⁺; rt: 0.33 min (LC/MS-method C).

### Step 9: tert-butyl (S)-(4-(2-(2-oxo-4-(trifluoromethyl)imidazolidin-1-yl)propan-2-yl)pyridin-2-yl)carbamate

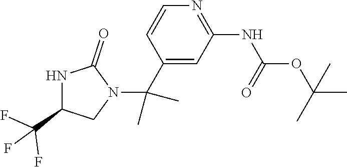 [SMILES: CC(C)(C)OC(=O)Nc1cc(C(C)(C)N2C[C@@H](C(F)(F)F)NC2=O)ccn1]

Following the procedure described for Int. 51, Step 3A, using (S)-1-(2-(2-chloropyridin-4-yl)propan-2-yl)-4-(trifluoromethyl)imidazolidin-2-one (2.0 g), tert-butyl (S)-(4-(2-(2-oxo-4-(trifluoromethyl)imidazolidin-1-yl)propan-2-yl)pyridin-2-yl)carbamate (1.75 g) was obtained as a yellow foam. LC/MS: m/z=389.0 [M+H]⁺; rt: 0.30 min (LC/MS-method C).

### Step 10: (S)-1-(2-(2-aminopyridin-4-yl)propan-2-yl)-4-(trifluoromethyl)imidazolidin-2-one

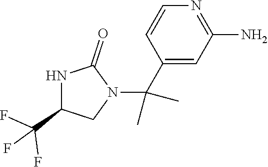 [SMILES: CC(C)(c1ccnc(N)c1)N1C[C@@H](C(F)(F)F)NC1=O]

Following the procedure described for Int. 51, Step 4A, using tert-butyl (S)-(4-(2-(2-oxo-4-(trifluoromethyl)imidazolidin-1-yl)propan-2-yl)pyridin-2-yl)carbamate (1.75 g), (S)-1-(2-(2-aminopyridin-4-yl)propan-2-yl)-4-(trifluoromethyl)imidazolidin-2-one (1.22 g) was obtained as a yellow foam. LC/MS: m/z=289.0 [M+H]⁺; rt: 0.17 min (LC/MS-method C).

### Step 11: tert-butyl ((S)-1-(4,4-difluorocyclohexyl)-2-oxo-2-((4-(2-((S)-2-oxo-4-(trifluoromethyl)imidazolidin-1-yl)propan-2-yl)pyridin-2-yl)amino)ethyl)carbamate

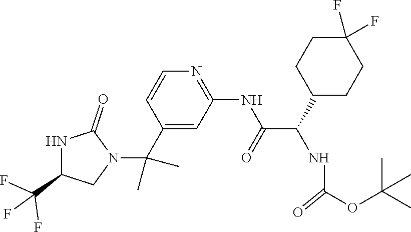

Following the procedure described for Int. 51, Step 5A, using (S)-1-(2-(2-aminopyridin-4-yl)propan-2-yl)-4-(trifluoromethyl)imidazolidin-2-one (1.12 g), tert-butyl ((S)-1-(4,4-difluorocyclohexyl)-2-oxo-2-((4-(2-((S)-2-oxo-4-(trifluoromethyl)imidazolidin-1-yl)propan-2-yl)pyridin-2-yl)amino)ethyl)carbamate (1.0 g) was obtained as a colorless solid which was purified by chiral SFC (column: DAICEL CHIRALPAK IA (250 mm*30 mm, 10 um); mobile phase: [HEXANE-IPA (0.1% IPAM)]; B %: 20%, isocratic elution mode) and further purified by FC (SiO₂; Eluent of 0~100% EA/PE) again to afford 533 mg as a colorless solid. LC/MS: m/z=564.1 [M+H]⁺; rt: 0.48 min (LC/MS-method C).

### Step 12: (S)-2-amino-2-(4,4-difluorocyclohexyl)-N-(4-(2-((S)-2-oxo-4-(trifluoromethyl)imidazolidin-1-yl)propan-2-yl)pyridin-2-yl)acetamide

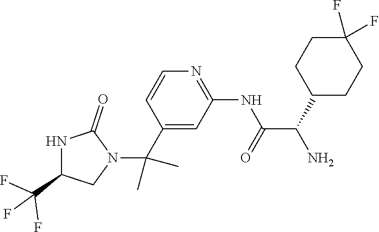

Following the procedure described for Int. 046, using tert-butyl ((S)-1-(4,4-difluorocyclohexyl)-2-oxo-2-((4-(2-((S)-2-oxo-4-(trifluoromethyl)imidazolidin-1-yl)propan-2-yl)pyridin-2-yl)amino)ethyl)carbamate (260 mg), afforded (S)-2-amino-2-(4,4-difluorocyclohexyl)-N-(4-(2-((S)-2-oxo-4-(trifluoromethyl)imidazolidin-1-yl)propan-2-yl)pyridin-2-yl)acetamide (170 mg). LC/MS: m/z=464.2 [M+H]⁺; rt: 1.24 min (LC/MS-method A).

### Intermediate 66: (S)-2-Amino-2-((1r,4S)-4-fluorocyclohexyl)-N-(4-(((S)-2-oxo-4-(trifluoromethyl)imidazolidin-1-yl)methyl)pyridin-2-yl)acetamide

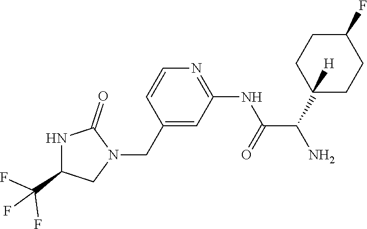

### Step 1: Benzyl (2-((4-(((tert-butyldimethylsilyl)oxy)methyl)pyridin-2-yl)amino)-1-(4-fluorocyclohexyl)-2-oxoethyl)carbamate

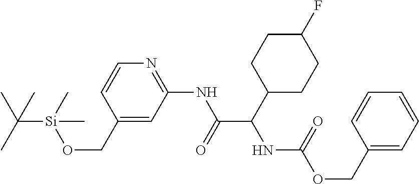 [SMILES: CC(C)(C)[Si](C)(C)OCc1ccnc(NC(=O)C(NC(=O)OCc2ccccc2)C2CCC(F)CC2)c1]

A mixture of 2-(((benzyloxy)carbonyl)amino)-2-(4-fluorocyclohexyl)acetic acid (I-062-8, 4 g), 4-(((tert-butyldimethylsilyl)oxy)methyl)pyridin-2-amine (Int. 1, Step A1, 4.01 g), DMAP (3.16 g), EDCl (4.96 g) in DCM (80 ml) was degassed, purged with N₂ for 3 times, and stirred at 25° C. for 12 h under N₂ atmosphere. The residue was poured into H₂O (40 ml) and the aqueous layer was extracted with DCM (50 ml, 3×). The organic layer was washed with brine (40 ml), dried over Na₂SO₄ and filtered. The filtrate was concentrated to give a residue which was purified by flash silica gel chromatography (ISCO®; 40 g SepaFlash® silica flash column, PE/EA:EA=0 to 20%; flow 30 ml/min) to yield 6.9 g of the title compound. LC/MS: m/z=530.8 [M+H]⁺; rt: 0.60 min (LC/MS-method E).

### Step 2-P1, -P2, -P3 and -P4: Benzyl ((S)-1-((1s,4R)-4-fluorocyclohexyl)-2-((4-(hydroxymethyl)pyridin-2-yl)amino)-2-oxoethyl)carbamate, benzyl ((S)-1-((1r,4S)-4-fluorocyclohexyl)-2-((4-(hydroxymethyl)pyridin-2-yl)amino)-2-oxoethyl)carbamate, benzyl ((R)-1-((1 s,4S)-4-fluorocyclohexyl)-2-((4-(hydroxymethyl)pyridin-2-yl)amino)-2-oxoethyl)carbamate, and benzyl ((R)-1-((1r,4R)-4-fluorocyclohexyl)-2-((4-(hydroxymethyl)pyridin-2-yl)amino)-2-oxoethyl)carbamate

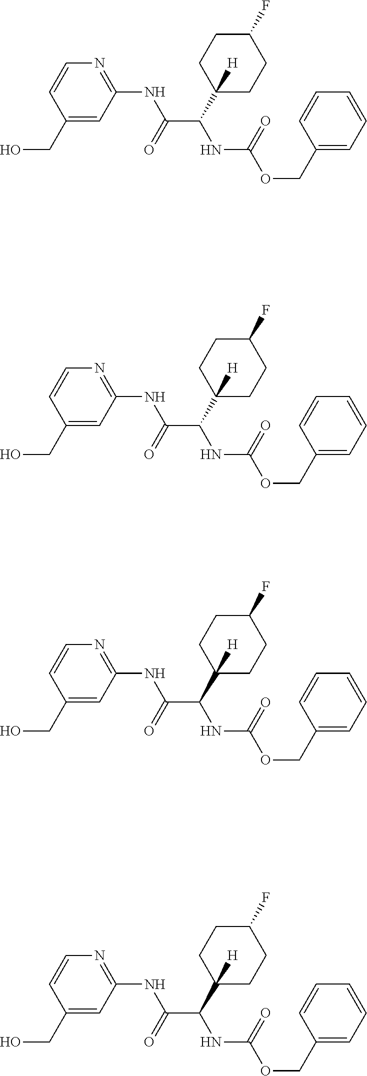

To benzyl (2-((4-(((tert-butyldimethylsilyl)oxy)methyl)pyridin-2-yl)amino)-1-(4-fluorocyclohexyl)-2-oxoethyl)carbamate (8.5 g) in THE (100 ml) was added TBAF (1 M in THF, 15.63 ml) and the mixture was stirred at 20° C. for 2 h. Then the residue was poured into H₂O (50 ml), the aqueous layer was extracted with EA (50 ml, 3×). The organic layer was washed with brine (50 ml), dried over Na₂SO₄ and filtered. The filtrate was concentrated to give a residue which was purified by flash silica gel chromatography (ISCO®; 40 g SepaFlash® silica flash column, PE/EA:EA=0 to 80%; flow 45 ml/min) to yield 4.8 g of a mixture of diastereomers. The diastereomers were separated by chiral preparative SFC (column: Daicel Chiralcel OJ-H (250 mm×30 mm, 5 μm); mobile phase: [A: CO₂, B: EtOH (0.1% NH₃ in H₂O)]; B: 25%, isocratic elution mode) to yield 0.98 g of P1, 1.05 g of P2, 1.15 g of P3 and 1.1 g of P4. Analytical SFC data: (Chiralcel OJ-3, 50 mm×4.6 mm, 3 μm; A: CO₂, B: EtOH (0.05% DEA); B: 5-40%, flow 3 ml/min, T: 35° C.): P1: rt=1.23 min, >99%; P2:1.36 min, 98%; P3:1.50 min, >99%; P4:1.62 min, >99%.

N.B.: The absolute stereochemistry of the four diastereomers was tentatively assigned. This assignment was kept in the downstream chemistry.

### Step 3-P2: Benzyl ((S)-1-((1r,4S)-4-fluorocyclohexyl)-2-((4-formylpyridin-2-yl)amino)-2-oxoethyl)carbamate

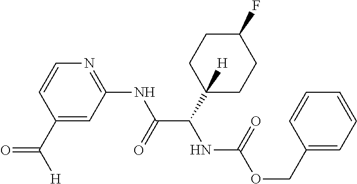

To a solution of benzyl ((S)-1-((1r,4S)-4-fluorocyclohexyl)-2-((4-(hydroxymethyl)pyridin-2-yl)amino)-2-oxoethyl)carbamate (Step 2-P2, 1.05 g) in dry DCM (20 ml) was added DMP (1.50 g) and the reaction mixture was stirred at 20° C. for 1 h. Then the reaction mixture was poured into H₂O (20 ml) and extracted with DCM (30 ml, 3×). The combined organic layers were washed with brine (20 ml), dried over Na₂SO₄, filtered and concentrated to give a residue which was purified by flash silica gel chromatography (ISCO®; 12 g SepaFlash® silica flash column, PE/EA:EA=0 to 40%; flow 30 ml/min) to yield 995 mg of the title compound. Analytical SFC data: (Chiralcel OJ-3, 50 mm×4.6 mm, 3 μm; A: CO₂, B: ACN (0.05% DEA); B: 5-40%, flow 3 ml/min, T: 35° C.): rt=1.53 min, 98%.

### Step 4-P2: Benzyl ((S)-2-((4-((((S)-2-amino-3,3,3-trifluoropropyl)amino)methyl)pyridin-2-yl)amino)-1-((1r4S)-4-fluorocyclohexyl)-2-oxoethyl)carbamate

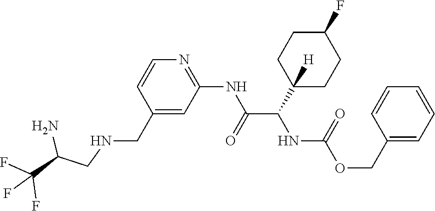

Benzyl ((S)-1-((1r,4S)-4-fluorocyclohexyl)-2-((4-formylpyridin-2-yl)amino)-2-oxoethyl)carbamate (300 mg) and (S)-3,3,3-trifluoropropane-1,2-diamine dihydrochloride (Int. 1, Step B6, 175 mg) were dissolved in DCM (5.5 ml) under Ar. After the addition of TEA (0.44 ml) the mixture was stirred for 5 min. Then the mixture was heated to 40° C. for 1 h. Then NaBH₃CN (160 mg), dry MeOH (2.5 ml) and AcOH (220 μl) were added at RT and the mixture was stirred for 2 h. After standing overnight the mixture was poured into sat. NaHCO₃ solution and the aqueous phase was extracted with DCM (3×). The combined organic phases were dried over Na₂SO₄, filtered and concentrated in vacuo. The residue was purified by preparative RP HPLC (flow: 25 ml/min; gradient: 100% H₂O+0.05% TFA for 20 min, 100% H₂O+0.05% TFA to 55% H₂O+0.05% TFA/45% ACN in 30 min; column: Purosphere® STAR-RP18, 25 mm×250 mm, 10 μm). The pure product containing fractions were combined, the ACN was partly removed and the TFA neutralised by conc. NaHCO₃ solution. After extracting the product with DCM (2×) the combined organic phases were dried over sodium sulphate, filtered and concentrated in vacuo. The residue was dissolved in ACN/H₂O and freeze dried to yield 285 mg of the title compound. LC/MS: m/z=526.1 [M+H]⁺; rt: 1.47 min (LC/MS-method A).

### Step 5-P2: Benzyl ((S)-1-((1r,4S)-4-fluorocyclohexyl)-2-oxo-2-((4-(((S)-2-oxo-4-(trifluoromethyl)imidazolidin-1-yl)methyl)pyridin-2-yl)amino)ethyl)carbamate

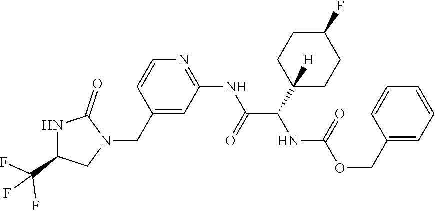

Benzyl ((S)-2-((4-((((S)-2-amino-3,3,3-trifluoropropyl)amino)methyl)pyridin-2-yl)amino)-1-((1r,4S)-4-fluorocyclohexyl)-2-oxoethyl)carbamate (283 mg) was dissolved in THE (14 ml) and the mixture was heated to 65° C. for 5 min. Then CDI (262 mg) was added and stirring was continued overnight at 65° C. The mixture was poured onto saturated NaHCO₃ solution and DCM was added. The organic layer was separated and the aqueous phase was extracted twice with DCM. The combined organic phases were dried over sodium sulphate, filtered and concentrated in vacuo. The residue was purified by silica gel column chromatography (12 g silica gel; gradient: DCM/EtOH: 100% DCM for 5 min, 100% to 90% DCM in 45 min; 90% DCM for 5 min), the combined product containing fractions were concentrated in vacuo and the residue was lyophilised from ACN/water to yield 236 mg of the title compound. LC/MS: m/z=552.1 [M+H]⁺; rt: 2.03 min (LC/MS-method A).

### Step 6: (S)-2-Amino-2-((1r,4S)-4-fluorocyclohexyl)-N-(4-(((S)-2-oxo-4-(trifluoromethyl)-imidazolidin-1-yl)methyl)pyridin-2-yl)acetamide

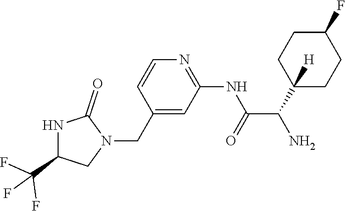

Benzyl ((S)-1-((1r,4S)-4-fluorocyclohexyl)-2-oxo-2-((4-(((S)-2-oxo-4-(trifluoromethyl)imidazolidin-1-yl)methyl)pyridin-2-yl)amino)ethyl)carbamate (232 mg) was dissolved in MeOH (6 ml) under Ar atmosphere. Pd/C (22 mg, 10%) was added and the flask was purged with H₂ from a balloon. After stirring for 4 h under a H₂ atmosphere the catalyst was filtered off and washed with MeOH. The filtrate was concentrated and the residue lyophilised from ACN/water to yield 172 mg of the crude title compound. LC/MS: m/z=418.2 [M+H]⁺; rt: 1.02 min (LC/MS-method A).

### Intermediate 67: (S)-2-amino-N-(4-((S)-1-(5,5-difluoro-2-oxotetrahydropyrimidin-1(2H)-yl)-2-methoxyethyl)pyridin-2-yl)-2-(4,4-difluorocyclohexyl)acetamide

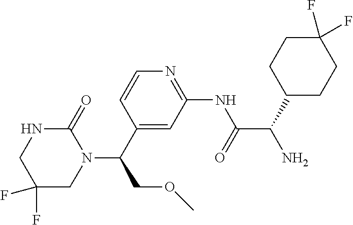

### Step 1: tert-butyl (S)-(1-(4,4-difluorocyclohexyl)-2-((4-(2-methoxyacetyl)pyridin-2-yl)amino)-2-oxoethyl)carbamate

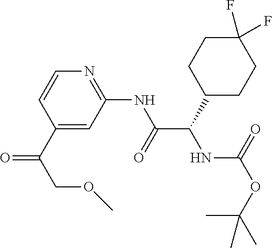

Prepared using the procedure described for Int. 17, Step 2, employing (S)-2-((tert-butoxycarbonyl)amino)-2-(4,4-difluorocyclohexyl)acetic acid. LC/MS: m/z=442.2 [M+H]⁺; rt: 0.42 min (LC/MS-method C).

### Step 2: tert-butyl ((1S)-2-((4-(1-((3-amino-2,2-difluoropropyl)amino)-2-methoxyethyl)pyridin-2-yl)amino)-1-(4,4-difluorocyclohexyl)-2-oxoethyl)carbamate

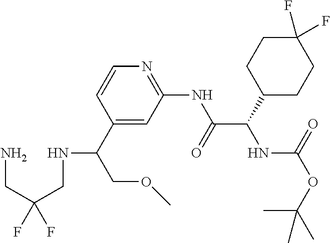 [SMILES: COCC(NCC(F)(F)CN)c1ccnc(NC(=O)[C@@H](NC(=O)OC(C)(C)C)C2CCC(F)(F)CC2)c1]

To a solution of tert-butyl (S)-(1-(4,4-difluorocyclohexyl)-2-((4-(2-methoxyacetyl)pyridin-2-yl)amino)-2-oxoethyl)carbamate (1 g) in i-PrOH (30 ml) was added TEA (946 μl) and 2,2-difluoropropane-1,3-diamine hydrochloride (539 mg) and the mixture was stirred at 60° C. for 12 hr before being concentrated and redissolved in MeOH (30 ml). Then NaBH₃CN (427 mg) was added and the mixture stirred at 25° C. for 10 min before AcOH (1.30 ml) was added dropwise and stirred at 25° C. for 50 min. The reaction mixture was added DCM (100 ml) and water (150 ml) and the pH was adjusted to 7~8 with NaHCO₃. The organic layers was separated and the aqueous phase was extracted with DCM (50 ml×2) and the combined organic layers were washed with brine (50 ml×3), dried over anhydrous Na₂SO₄, filtered and concentrated to afford tert-butyl ((1S)-2-((4-(1-((3-amino-2,2-difluoropropyl)amino)-2-methoxyethyl)pyridin-2-yl)amino)-1-(4,4-difluorocyclohexyl)-2-oxoethyl)carbamate (1.21 g) as a yellow solid that was used directly without further purification. LC/MS: m/z=536.1 [M+H]⁺; rt: 0.31 min (LC/MS-method C).

### Step 3: tert-butyl ((S)-2-((4-((S)-1-(5,5-difluoro-2-oxotetrahydropyrimidin-1(2H)-yl)-2-methoxyethyl)pyridin-2-yl)amino)-1-(4,4-difluorocyclohexyl)-2-oxoethyl)carbamate

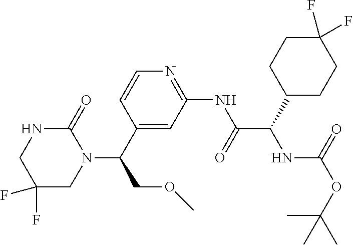

To a solution of tert-butyl ((1S)-2-((4-(1-((3-amino-2,2-difluoropropyl)amino)-2-methoxyethyl)pyridin-2-yl)amino)-1-(4,4-difluorocyclohexyl)-2-oxoethyl)carbamate (1.21 g) in THE (46 ml) was added CDI (1.10 g) at 65° C. and the mixture stirred at 65° C. for 1 hr. Then additional CDI (1.10 g) was added and the stirring continued at 65° C. for 0.5 hr before addition of NaOH (2M, 20 ml, aq.) at 25° C. and stirred for 10 min. Then the mixture was heated to 65° C. and stirred for 10 min before being concentrated under reduced pressure. The residue was diluted with EA:DCM (250 ml, 10:1) and washed with brine (100 ml×4). The combined organic layers were dried over Na₂SO₄, filtered, and concentrated under reduced pressure to afford the crude product, which was purified by FC (SiO₂, Eluent of 0~50~100% EA/PE) to afford the product diastereomer mixture as a white foam (650 mg). The diastereomers were separated by chiral SFC (column: DAICEL CHIRALPAK IG (250 mm*30 mm, 10 um); mobile phase: [CO₂-EtOH (0.1% NH3H2O)]; B %: 45%, isocratic elution mode) to afford tert-butyl ((S)-2-((4-((S)-1-(5,5-difluoro-2-oxotetrahydropyrimidin-1(2H)-yl)-2-methoxyethyl)pyridin-2-yl)amino)-1-(4,4-difluorocyclohexyl)-2-oxoethyl)carbamate (254 mg) as the second peak eluting. LC/MS: m/z=562.2 [M+H]⁺; rt: 0.36 min (LC/MS-method C).

### Step 4: (S)-2-amino-N-(4-((S)-1-(5,5-difluoro-2-oxotetrahydropyrimidin-1(2H)-yl)-2-methoxyethyl)pyridin-2-yl)-2-(4,4-difluorocyclohexyl)acetamide

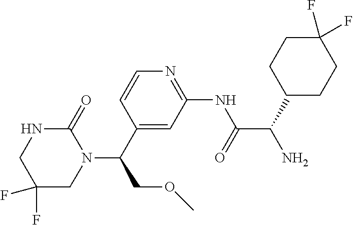

Following the procedure described for Int. 46, using tert-butyl ((S)-2-((4-((S)-1-(5,5-difluoro-2-oxotetrahydropyrimidin-1(2H)-yl)-2-methoxyethyl)pyridin-2-yl)amino)-1-(4,4-difluorocyclohexyl)-2-oxoethyl)carbamate (250 mg), (S)-2-amino-N-(4-((S)-1-(5,5-difluoro-2-oxotetrahydropyrimidin-1(2H)-yl)-2-methoxyethyl)pyridin-2-yl)-2-(4,4-difluorocyclohexyl)acetamide (195 mg) was obtained. LC/MS: m/z=462.2 [M+H]⁺; rt: 1.12 min (LC/MS-method A).

### Intermediate 68: N-(1-(2-((S)-2-amino-2-(4,4-difluorocyclohexyl)acetamido)pyridin-4-yl)propyl)-4,4,4-trifluorobutanamide

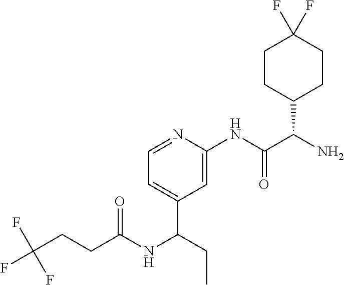 [SMILES: CCC(NC(=O)CCC(F)(F)F)c1ccnc(NC(=O)[C@@H](N)C2CCC(F)(F)CC2)c1]

### Step 1: tert-butyl (4-(1-(((R)-tert-butylsulfinyl)amino)propyl)pyridin-2-yl)carbamate

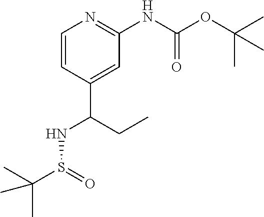 [SMILES: CCC(N[S@](=O)C(C)(C)C)c1ccnc(NC(=O)OC(C)(C)C)c1]

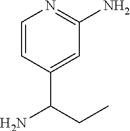

A mixture of tert-butyl (4-(1-(((R)-tert-butylsulfinyl)amino)propyl)pyridin-2-yl)carbamate (16 g) in HCl/dioxane (320 ml) was degassed and purged with nitrogen 3 times, and then stirred at 40° C. for 2 hr and then 60° C. for 2 hr. under N₂ atmosphere. The solvent was removed in vacuo to give 4-(1-aminopropyl)pyridin-2-amine (14 g) as colorless solid. ¹H NMR (400 MHz, CDCl₃) δ ppm: 8.96 (br s, 2H), 8.03 (d, J=6.6 Hz, 1H), 7.14-7.01 (m, 1H), 2.05-1.89 (m, 1H), 1.89-1.76 (m, 1H), 1.23-0.99 (m, 3H), 0.82 (t, J=7.4 Hz, 2H).

### Step 3: tert-butyl (1-(2-aminopyridin-4-yl)propyl)carbamate-enantiomer A+B

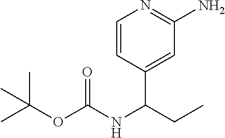 [SMILES: CCC(NC(=O)OC(C)(C)C)c1ccnc(N)c1]

To a solution of 4-(1-aminopropyl)pyridin-2-amine hydrochloride (11 g) in MeOH (110 ml) was added Boc₂O (4.85 g) and add TEA (7.73 mL) and the mixture was stirred at 0° C. for 1 hr under N₂ atmosphere. NaOH (200 ml, 2M, aq.) was added and extracted with ethyl acetate (200 ml×3). The combined organic phase was washed with brine (200 ml×3), dried with anhydrous Na₂SO₄, filtered and concentrated in vacuum, to afford the crude product (4.2 g) as a yellow oil. The enantiomers were separated by chiral SFC (column: REGIS(S,S)WHELK-O1 (250 mm*25 mm, 10 um); mobile phase: [CO₂-i-PrOH (0.1% NH3H2O)]; B %: 35%, isocratic elution mode) to afford:

Enantiomer A eluting first: 1.7 g, yellow oil. ¹H NMR (400 MHz, CDCl3) δ ppm: 8.55-8.46 (m, 1H), 8.00-7.85 (m, 1H), 6.59-6.33 (m, 2H), 4.90-4.80 (m, 1H), 1.80-1.61 (m, 2H), 1.42 (br s, 9H), 1.20 (d, J=6.1 Hz, 1H), 0.90 (t, J=7.4 Hz, 3H); and

Enantiomer B eluting second: 2.5 g, yellow oil. ¹H NMR (400 MHz, CDCl3) δ ppm: 8.56-8.50 (m, 1H), 7.97 (d, J=5.4 Hz, 1H), 6.61-6.41 (m, 1H), 6.41 (s, 1H), 4.88 (br d, J=7.6 Hz, 1H), 1.80-1.63 (m, 2H), 1.50-1.14 (m, 9H), 0.91 (t, J=7.4 Hz, 3H).

### Step 4: tert-butyl (1-(2-((S)-2-(((benzyloxy)carbonyl)amino)-2-(4,4-difluorocyclohexyl)acetamido)pyridin-4-yl)propyl)carbamate

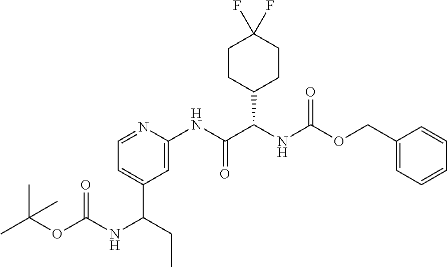 [SMILES: CCC(NC(=O)OC(C)(C)C)c1ccnc(NC(=O)[C@@H](NC(=O)OCc2ccccc2)C2CCC(F)(F)CC2)c1]

To a solution of 2-(((benzyloxy)carbonyl)amino)-2-(4,4-difluorocyclohexyl)acetic acid (1.82 g) was added tert-butyl (1-(2-aminopyridin-4-yl)propyl)carbamate-enantiomer B, DIPEA (2.91 mL), EDCl (5.54 g) and DMAP (5.43 g) in DCM (20 ml) and then the mixture was stirred at RT for 12 hr under a nitrogen atmosphere. Water was added and the aqueous phase was extracted with EA (50 ml×3) and the combined organic phase was washed with brine (50 ml×3), 0.5M HCl (5 ml×10), dried over anhydrous Na₂SO₄, filtered and concentrated in vacuum to afford the crude product (2.8 g) as yellow solid. The diastereomers were separated by chiral SFC (REGIS(S,S)WHELK-O1 (250 mm*25 mm, 10 um); mobile phase: [CO₂-EtOH (0.1% NH₃H₂O)]; B %: 30%, isocratic elution mode) to afford tert-butyl (1-(2-((S)-2-(((benzyloxy)carbonyl)amino)-2-(4,4-difluorocyclohexyl)acetamido)pyridin-4-yl)propyl)carbamate (1.38 g) as the second eluting peak. LC/MS: m/z=561.2 [M+H]⁺; rt: 2.54 min (LC/MS-method A).

### Step 5: benzyl ((1S)-2-((4-(1-aminopropyl)pyridin-2-yl)amino)-1-(4,4-difluorocyclohexyl)-2-oxoethyl)carbamate

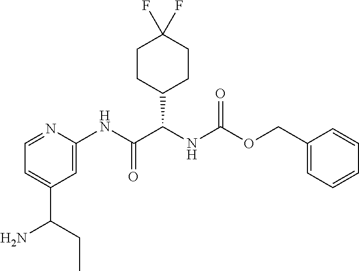 [SMILES: CCC(N)c1ccnc(NC(=O)[C@@H](NC(=O)OCc2ccccc2)C2CCC(F)(F)CC2)c1]

Following the procedure described for Int. 57, Step 7, using tert-butyl (1-(2-((S)-2-(((benzyloxy)carbonyl)amino)-2-(4,4-difluorocyclohexyl)acetamido)pyridin-4-yl)propyl)carbamate (655 mg), afforded benzyl ((1S)-2-((4-(1-aminopropyl)pyridin-2-yl)amino)-1-(4,4-difluorocyclohexyl)-2-oxoethyl)carbamate (540 mg) that was used without further purification in the next step. LC/MS: m/z=461.1 [M+H]⁺; rt: 1.46 min (LC/MS-method A).

### Step 6: benzyl ((1S)-1-(4,4-difluorocyclohexyl)-2-oxo-2-((4-(1-(4,4,4-trifluorobutanamido)propyl)pyridin-2-yl)amino)ethyl)carbamate

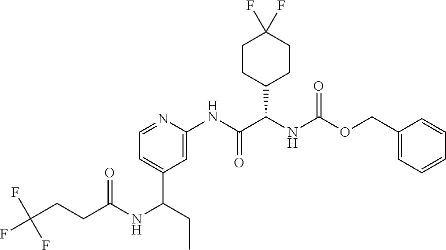 [SMILES: CCC(NC(=O)CCC(F)(F)F)c1ccnc(NC(=O)[C@@H](NC(=O)OCc2ccccc2)C2CCC(F)(F)CC2)c1]

Following the procedure described for Int. 57, Step 8, using benzyl ((1S)-2-((4-(1-aminopropyl)pyridin-2-yl)amino)-1-(4,4-difluorocyclohexyl)-2-oxoethyl)carbamate (134 mg), afforded benzyl ((1S)-1-(4,4-difluorocyclohexyl)-2-oxo-2-((4-(1-(4,4,4-trifluorobutanamido)propyl)pyridin-2-yl)amino)ethyl)carbamate (165 mg). LC/MS: m/z=585.1 [M+H]⁺; rt: 2.33 min (LC/MS-method A).

### Step 7: N-(1-(2-((S)-2-amino-2-(4,4-difluorocyclohexyl)acetamido)pyridin-4-yl)propyl)-4,4,4-trifluorobutanamide

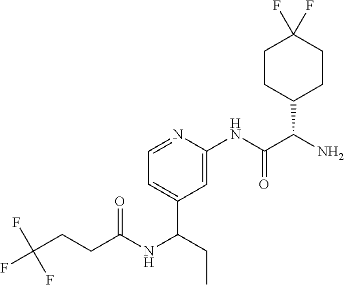 [SMILES: CCC(NC(=O)CCC(F)(F)F)c1ccnc(NC(=O)[C@@H](N)C2CCC(F)(F)CC2)c1]

Following the procedure described for Int. 57, using benzyl ((1S)-1-(4,4-difluorocyclohexyl)-2-oxo-2-((4-(1-(4,4,4-trifluorobutanamido)propyl)pyridin-2-yl)amino)ethyl)carbamate (165 mg) afforded N-(1-(2-((S)-2-amino-2-(4,4-difluorocyclohexyl)acetamido)pyridin-4-yl)propyl)-4,4,4-trifluorobutanamide (120 mg).

### Intermediate 69-DS1, -DS2, -DS4: 2-Amino-2-(4-fluoro-4-methylcyclohexyl)-N-(4-((S)-2-methoxy-1-((S)-2-oxo-4-(trifluoromethyl)imidazolidin-1-yl)ethyl)pyridin-2-yl)acetamide hydrochloride

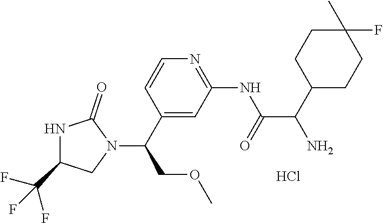 [SMILES: COC[C@H](c1ccnc(NC(=O)C(N)C2CCC(C)(F)CC2)c1)N1C[C@@H](C(F)(F)F)NC1=O ; Cl]

### Step 1: 8-Methyl-1,4-dioxaspiro[4.5]decan-8-ol

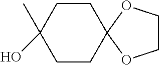

To a solution of 1,4-dioxaspiro[4.5]decan-8-one (24 g) in THE (720 ml) was added MeMgBr (3 M in diethyl ether, 76.84 ml) at 0° C. under N₂ atmosphere. The solution was stirred at 0° C. for 10 min. Then the reaction was quenched with sat. aqueous NH₄Cl solution (600 ml) and extracted with EA (500 ml, 3×). The organic phase was washed with brine, dried over anhydrous Na₂SO₄, filtered and concentrated in vacuo to yield 26.47 g of the title compound.

### Step 2: 8-Fluoro-8-methyl-1,4-dioxaspiro[4.5]decane

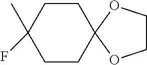

To a solution of 8-methyl-1,4-dioxaspiro[4.5]decan-8-ol (30 g) in DCM (800 ml) was added DAST (46 ml) at 0° C. and the solution was stirred at 25° C. for 12 h. Then the reaction was quenched with sat. aqueous NaHCO₃ (500 ml) and extracted with EA. The organic phase was washed with brine, dried over anhydrous Na₂SO₄ and concentrated under reduced pressure to give a residue, which was purified by flash silica gel chromatography (ISCO®; 80 g SepaFlash® silica flash column, PE/EA:EA from 0 to 10%, flow: 100 ml/min) to yield 7.2 g of the title compound. ¹H NMR (400 MHz, CDCl₃) δ ppm: 3.98-3.96 (m, 4H), 1.90-1.87 (m, 2H), 1.80-1.74 (m, 3H), 1.72-1.64 (m, 3H), 1.35 (d, 3H).

### Step 3: 4-Fluoro-4-methylcyclohexan-1-one

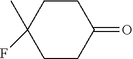

A solution of 8-fluoro-8-methyl-1,4-dioxaspiro[4.5]decane (6.6 g) and pyridine, 4-methylbenzenesulfonate (19.04 g) in acetone (200 ml) and H₂O (200 ml) was stirred at 65° C. for 16 h. Then the reaction mixture was partitioned between EA and H₂O, and the layers were separated. The aqueous layer was extracted with EA, and the combined organic fractions were washed with brine, dried over anhydrous Na₂SO₄, filtered concentrated under reduced pressure to yield 3.97 g of the title compound.

### Step 4: Methyl 2-((tert-butoxycarbonyl)amino)-2-(4-fluoro-4-methylcyclohexylidene)acetate

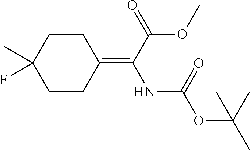 [SMILES: COC(=O)C(NC(=O)OC(C)(C)C)=C1CCC(C)(F)CC1]

To a solution of methyl 2-(tert-butoxycarbonylamino)-2-dimethoxyphosphoryl-acetate (9.59 g) in NMP (45 ml) was dropwise added DBU (4.54 ml) at 15° C. and the mixture was stirred at this temperature for 30 min. Then a solution of 4-fluoro-4-methylcyclohexan-1-one (4 g) in NMP (45 ml) was added dropwise. The resulting mixture was stirred at 15° C. for 4 h. Then the mixture was poured into H₂O, stirred for 10 min and filtered. The filter cake was dried under reduced pressure to yield 2 g of the title compound.

### Step 5: Methyl 2-((tert-butoxycarbonyl)amino)-2-(4-fluoro-4-methylcyclohexyl)acetate

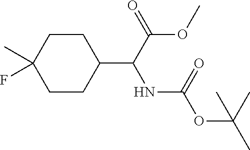 [SMILES: COC(=O)C(NC(=O)OC(C)(C)C)C1CCC(C)(F)CC1]

To a solution of methyl 2-((tert-butoxycarbonyl)amino)-2-(4-fluoro-4-methylcyclohexylidene)acetate (4.1 g) in MeOH (120 ml) was added Pd/C (1 g, 10%). The mixture was stirred under H₂ at 25° C. for 12 h. Then the reaction mixture was filtered and concentrated under reduced pressure to yield 4.3 g of the title compound.

### Step 6: 2-((Tert-butoxycarbonyl)amino)-2-(4-fluoro-4-methylcyclohexyl)acetic acid

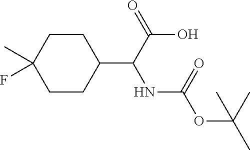 [SMILES: CC1(F)CCC(C(NC(=O)OC(C)(C)C)C(=O)O)CC1]

To a solution of methyl 2-((tert-butoxycarbonyl)amino)-2-(4-fluoro-4-methylcyclohexyl)acetate (3.3 g) in MeOH (30 ml) was added aqueous LiOH·H₂O solution (1 M, 10.88 ml) and the mixture was stirred at 60° C. for 2 h. Then the solution was poured into water and the MeOH was removed in vacuo. The residue was acidified by aqueous HCl (1 M) to pH=2-3 and extracted with DCM. The organic phase was washed with brine, dried over anhydrous Na₂SO₄, filtered and concentrated in vacuo to yield 2.51 g of the title compound.

### Step 7-P1, -P2, -P3, -P4: Tert-butyl (1-(4-fluoro-4-methylcyclohexyl)-2-((4-((S)-2-methoxy-1-((S)-2-oxo-4-(trifluoromethyl)imidazolidin-1-yl)ethyl)pyridin-2-yl)amino)-2-oxoethyl)carbamate

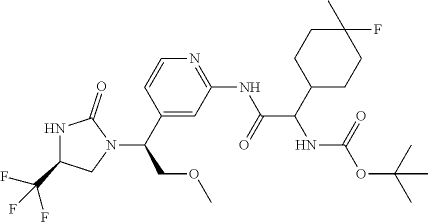 [SMILES: COC[C@H](c1ccnc(NC(=O)C(NC(=O)OC(C)(C)C)C2CCC(C)(F)CC2)c1)N1C[C@@H](C(F)(F)F)NC1=O]

To a solution of 2-((tert-butoxycarbonyl)amino)-2-(4-fluoro-4-methylcyclohexyl)acetic acid (1.14 g) and (S)-1-((S)-1-(2-aminopyridin-4-yl)-2-methoxyethyl)-4-(trifluoromethyl)imidazolidin-2-one hydrochloride (Int. 54-B-P1; 1.00 g) in DCM (10 ml) were added EDCl (1.89 g) and DMAP (1.61 g). The mixture was stirred at 40° C. for 20 min. Then aqueous NaOH (5 M, 10 ml) was added and the mixture was stirred at 25° C. for 12 h. Water (200 ml) was added and the aqueous phase was extracted with DCM (100 ml, 3×). The combined organic phases were dried over Na₂SO₄, filtered and concentrated under reduced pressure to give a residue which was purified by prep-HPLC (column: Phenomenex luna C18, 150×25 mm; 10 μm; mobile phase: [A: water (0.225% FA); B: ACN; gradient: B: 37%-67% B in 10 min) to yield 0.8 g of the title compound as a mixture of diastereomers. The mixture was separated by preparative SFC (column: Daicel Chiralpak IE (50 mm×250 mm; 10 μm); mobile phase: [A: nHep, B: EtOH (0.1% TFA)]; gradient: 30%-50% B in 20 min; 50 ml/min) to yield ~91 mg of P1, ~36 mg of P2, ~32 mg of P3 and 77 mg of P4. Since P1, P2 and P3 were impure further SFC purifications were required: P1: 54 mg (column: Daicel Chiralpak AD (250 mm×30 mm, 10 μm); mobile phase: [A: CO₂, B: EtOH]; B: 35% isocratic mode, flow 50 ml/min). P2: 12 mg (Daicel Chiralpak AD (250 mm×30 mm, 10 μm); mobile phase: [A: CO₂, B: IPA (0.1% NH₃ in H₂O)]; B: 35%, isocratic elution mode; flow 50 ml/min). P3: 12 mg (Daicel Chiralpak (250 mm×30 mm, 10 μm); mobile phase: [A: CO₂, B: IPA (0.1% NH₃ in H₂O)]; B: 35%, isocratic elution mode; flow 50 ml/min). Analytical SFC data: (Chiralpak AD-3, 50 mm×4.6 mm, 3 μm; A: CO₂, B: IPA (0.05% DEA); B: 5-40%, flow 3 ml/min, T: 35° C.): P1: rt=1.75 min, 98.7%; P2: 2.03 min, 98.2%; P3: 2.21 min, >99%; P4: 2.44 min, >99%.

### Step 8: 2-Amino-2-(4-fluoro-4-methylcyclohexyl)-N-(4-((S)-2-methoxy-1-((S)-2-oxo-4-(trifluoromethyl)imidazolidin-1-yl)ethyl)pyridin-2-yl)acetamide hydrochloride—DS1

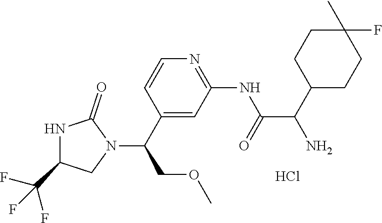 [SMILES: COC[C@H](c1ccnc(NC(=O)C(N)C2CCC(C)(F)CC2)c1)N1C[C@@H](C(F)(F)F)NC1=O ; Cl]

Tert-butyl (1-(4-fluoro-4-methylcyclohexyl)-2-((4-((S)-2-methoxy-1-((S)-2-oxo-4-(trifluoromethyl)imidazolidin-1-yl)ethyl)pyridin-2-yl)amino)-2-oxoethyl)carbamate (Step 7-P1, 44 mg) was dissolved in dry dioxane (0.5 ml) and HCl solution (0.5 ml; 4 M in dioxane) was added dropwise with stirring. After stirring for 1 h at RT the mixture was left standing overnight, then concentrated in vacuo and the residue was lyophilised from ACN/H₂O to yield 42 mg of the crude title compound. LC/MS: m/z=476.2 [M+H]⁺; rt: 1.16 min (LC/MS-method A).

### Step 8: 2-Amino-2-(4-fluoro-4-methylcyclohexyl)-N-(4-((S)-2-methoxy-1-((S)-2-oxo-4-(trifluoromethyl)imidazolidin-1-yl)ethyl)pyridin-2-yl)acetamide hydrochloride—DS2 & DS4

Following the procedure described for DS1, using Step 7-P2 (9 mg) and Step 7-P4 (74 mg) respectively, gave crude products requiring purification by preparative HPLC (flow: 15 ml/min; gradient: 95% H₂O+0.05% TFA/5% ACN in 45 min to 10% H₂O+0.05% TFA/90% ACN; column: XBridge, BEH130, Prep. C18, OBD; 19 mm×250 mm, 10 μm). 3 mg of DS2 and 13 mg of DS4 were obtained.

### Intermediate 70: (S)-2-amino-2-((1r,4S)-4-methylcyclohexyl)-N-(4-((R)-1-((S)-2-oxo-4-(trifluoromethyl)imidazolidin-1-yl)ethyl)pyridin-2-yl)acetamide

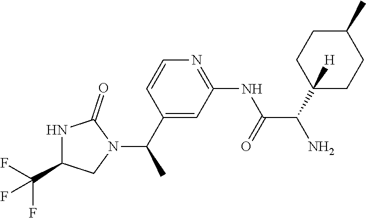

Was prepared by the synthesis route described for Int. 48-DS2, using methylmagnesium bromide and (S)-2-((tert-butoxycarbonyl)amino)-2-((1r,4S)-4-methylcyclohexyl)acetic acid in the respective steps, 210 mg of the title compound was obtained. LC/MS: m/z=428.2 [M+H]⁺; rt: 1.31 min (LC/MS-method A).

### Intermediate 71: N-(1-(2-((S)-2-amino-2-((1r,4S)-4-methylcyclohexyl)acetamido)-pyridin-4-yl)-2-methoxyethyl)-4,4,4-trifluorobutanamide

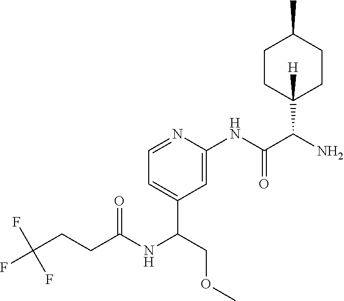

### Step 1: tert-butyl (4-(1-amino-2-methoxyethyl)pyridin-2-yl)carbamate

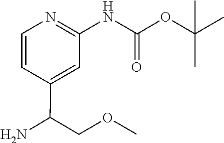 [SMILES: COCC(N)c1ccnc(NC(=O)OC(C)(C)C)c1]

To a solution of Tert-butyl (4-(2-methoxyacetyl)pyridin-2-yl)carbamate (Int. 54, Step B-3; 1.7 g) in MeOH (35 ml) was added ammonium acetate (4.92 g) and the mixture stirred at 45° C. for 6 hr before addition of NaBH₃CN (602 mg) was added and the mixture was stirred at 40° C. for 12 hr. Then the reaction mixture was diluted with water (100 ml) and extracted with EA (40 mL×3). The combined organic layers were washed with brine (50 ml×3), dried over Na₂SO₄, filtered and concentrated under reduced pressure to give I-070-1: tert-butyl (4-(1-amino-2-methoxyethyl)pyridin-2-yl)carbamate (1.63 g) as a yellow solid that was used without further purification. LC/MS: m/z=212.4 [M−56+H]+; rt: 0.21 min (LC/MS-method C).

### Step 2: tert-butyl (S)-(4-(2-methoxy-1-(4,4,4-trifluorobutanamido)ethyl)pyridin-2-yl)carbamate

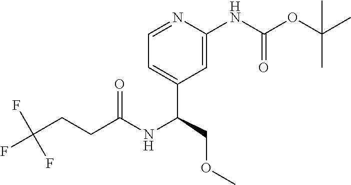

To a solution of 4,4,4-trifluorobutanoic acid (1.30 g), and DIPEA (2.66 ml) in DCM (10 ml) was added HOBt (1.32 g) and EDCl (1.87 g) at 0° C. and stirred at 25° C. for 1 hr before tert-butyl (4-(1-amino-2-methoxyethyl)pyridin-2-yl)carbamate (1.63 g) in DCM (16 ml) was added and the mixture was stirred at 25° C. for 12 hr. The reaction mixture was evaporated and the residue was purified by FC (SiO₂; Eluent of 0~50% EA/PE) to afford the product as a enantiomer mixture (1.4 g) as a yellow solid. The enantiomers were separated by chiral SFC (column: REGIS (R,R)WHELK-O1 (250 mm*25 mm, 10 um); mobile phase: [CO₂-i-PrOH (0.1% NH₃—H₂O)]; B %: 35%, isocratic elution mode) to give tert-butyl (S)-(4-(2-methoxy-1-(4,4,4-trifluorobutanamido)ethyl)pyridin-2-yl)carbamate (700 mg, colorless solid) as the first eluting enantiomer. LC/MS: m/z=392.3 [M−56+H]+; rt: 0.29 min (LC/MS-method C).

### Step 3: N-(1-(2-((S)-2-amino-2-((1r,4S)-4-methylcyclohexyl)acetamido)pyridin-4-yl)-2-methoxyethyl)-4,4,4-trifluorobutanamide

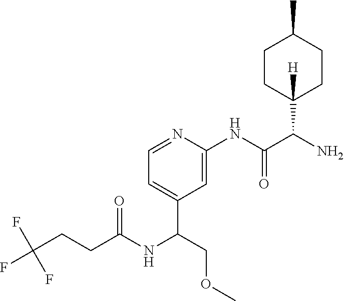

Following the synthesis steps described in Int. 65, Step 10, Step 11 and Step 12, using tert-butyl (S)-(4-(2-methoxy-1-(4,4,4-trifluorobutanamido)ethyl)pyridin-2-yl)carbamate and (S)-2-((tert-butoxycarbonyl)amino)-2-((1r,4S)-4-methylcyclohexyl)acetic acid, afforded N-(1-(2-((S)-2-amino-2-((1r,4S)-4-methylcyclohexyl)acetamido)pyridin-4-yl)-2-methoxyethyl)-4,4,4-trifluorobutanamide (175 mg). LC/MS: m/z=445.2 [M+H]⁺; rt: 1.35 min (LC/MS-method C).

### Intermediate 72: (S)-2-amino-2-(3,3-difluorocyclobutyl)-N-(4-((S)-2-methoxy-1-((S)-2-oxo-4-(trifluoromethyl)imidazolidin-1-yl)ethyl)pyridin-2-yl)acetamide

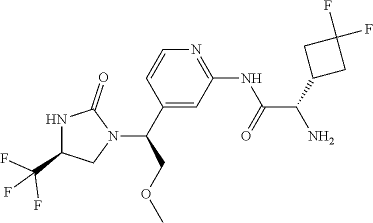 [SMILES: COC[C@H](c1ccnc(NC(=O)[C@@H](N)C2CC(F)(F)C2)c1)N1C[C@@H](C(F)(F)F)NC1=O]

Was prepared by the synthesis route described for Int. 52, using (S)-2-((tert-butoxycarbonyl)amino)-2-(3,3-difluorocyclobutyl)acetic acid, to afford (S)-2-amino-2-(3,3-difluorocyclobutyl)-N-(4-((S)-2-methoxy-1-((S)-2-oxo-4-(trifluoromethyl)imidazolidin-1-yl)ethyl)pyridin-2-yl)acetamide (350 mg). LC/MS: m/z=452.1 [M+H]⁺; rt: 1.12 min (LC/MS-method A).

### Intermediate 73: (2S)-2-amino-N-(4-((S)-2-methoxy-1-((S)-2-oxo-4-(trifluoromethyl)-imidazolidin-1-yl)ethyl)pyridin-2-yl)-2-(4-(trifluoromethyl)cyclohexyl)acetamide

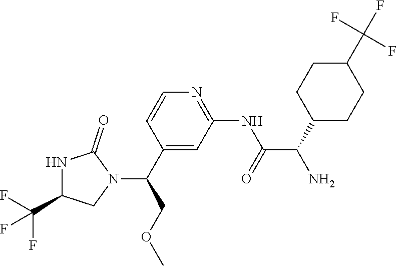 [SMILES: COC[C@H](c1ccnc(NC(=O)[C@@H](N)C2CCC(C(F)(F)F)CC2)c1)N1C[C@@H](C(F)(F)F)NC1=O]

Was prepared by the synthesis route described for Int. 52, using (2S)-2-((tert-butoxycarbonyl)amino)-2-(4-(trifluoromethyl)cyclohexyl)acetic acid, to afford (2S)-2-amino-N-(4-((S)-2-methoxy-1-((S)-2-oxo-4-(trifluoromethyl)imidazolidin-1-yl)ethyl)pyridin-2-yl)-2-(4-(trifluoromethyl)cyclohexyl)acetamide (150 mg). LC/MS: m/z=512.2 [M+H]⁺; rt: 1.34 min (LC/MS-method A).

### Intermediate 74: (S)-2-amino-2-((S)-3,3-difluorocyclohexyl)-N-(4-((S)-2-methoxy-1-((S)-2-oxo-4-(trifluoromethyl)imidazolidin-1-yl)ethyl)pyridin-2-yl)acetamide

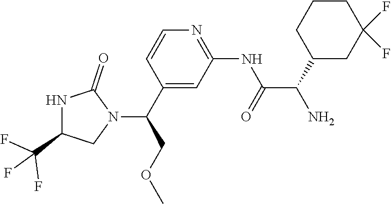 [SMILES: COC[C@H](c1ccnc(NC(=O)[C@@H](N)C2CCCC(F)(F)C2)c1)N1C[C@@H](C(F)(F)F)NC1=O]

### Step 1: tert-butyl ((S)-1-((S)-3,3-difluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)carbamate Diastereomer 1+2

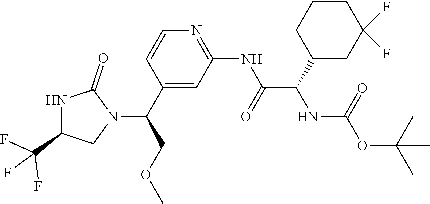 [SMILES: COC[C@H](c1ccnc(NC(=O)[C@@H](NC(=O)OC(C)(C)C)C2CCCC(F)(F)C2)c1)N1C[C@@H](C(F)(F)F)NC1=O]

Was prepared by the synthesis route described for Int. 52, Step 1, using (S)-2-((tert-butoxycarbonyl)amino)-2-((S)-3,3-difluorocyclohexyl)acetic acid, and the resultant diastereomers were separated by chiral SFC. First SFC: column: DAICEL CHIRALPAK AD (250 mm*30 mm, 10 um); mobile phase: [CO₂-i-PrOH (0.1% NH₃H₂O)]; B %: 35%, isocratic elution mode. Second SFC: column: REGIS(S,S)WHELK-O1 (250 mm*25 mm, 10 um); mobile phase: [CO₂-MeOH]; B %: 30%, isocratic elution mode. Third SFC: column: REGIS(S,S)WHELK-O1 (250 mm*25 mm, 10 um); mobile phase: [CO₂-i-PrOH (0.1% NH₃H₂O)]; B %: 35%, isocratic elution mode and column: DAICEL CHIRALCEL OX (250 mm*30 mm, 10 um); mobile phase: [CO₂-MeOH (0.1% NH₃H₂O)]; B %: 20%, isocratic elution mode. Fourth SFC: column: DAICEL CHIRALPAK IC (250 mm*30 mm, 10 um); mobile phase: [CO2-MeOH]; B %: 25%, isocratic elution mode. Diastereomer 1: LC/MS: m/z=580.2 [M+H]⁺; rt: 0.43 min (LC/MS-method C). Diastereomer 2: LC/MS: m/z=580.3 [M+H]⁺; rt: 0.42 min (LC/MS-method C).

### Step 2: (S)-2-amino-2-((S)-3,3-difluorocyclohexyl)-N-(4-((S)-2-methoxy-1-((S)-2-oxo-4-(trifluoromethyl)imidazolidin-1-yl)ethyl)pyridin-2-yl)acetamide—Diastereomer 1

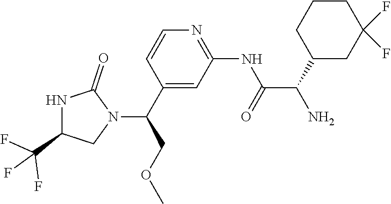 [SMILES: COC[C@H](c1ccnc(NC(=O)[C@@H](N)C2CCCC(F)(F)C2)c1)N1C[C@@H](C(F)(F)F)NC1=O]

Following the procedure described for Int. 46, using tert-butyl ((S)-1-((S)-3,3-difluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)carbamate diastereomer 1 (150 mg), afforded (S)-2-amino-2-((S)-3,3-difluorocyclohexyl)-N-(4-((S)-2-methoxy-1-((S)-2-oxo-4-(trifluoro-methyl)imidazolidin-1-yl)ethyl)pyridin-2-yl)acetamide diastereomer 1 (115 mg). LC/MS: m/z=480.2 [M+H]⁺; rt: 1.16 min (LC/MS-method A).

### Step 2: (S)-2-amino-2-((S)-3,3-difluorocyclohexyl)-N-(4-((S)-2-methoxy-1-((S)-2-oxo-4-(trifluoromethyl)imidazolidin-1-yl)ethyl)pyridin-2-yl)acetamide—Diastereomer 2

Following the procedure described for Int. 46, using tert-butyl ((S)-1-((S)-3,3-difluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)carbamate diastereomer 2 (162 mg) afforded (S)-2-amino-2-((S)-3,3-difluorocyclohexyl)-N-(4-((S)-2-methoxy-1-((S)-2-oxo-4-(trifluoro-methyl)imidazolidin-1-yl)ethyl)pyridin-2-yl)acetamide diastereomer 2 (125 mg). LC/MS: m/z=480.2 [M+H]⁺; rt: 1.16 min (LC/MS-method A).

### Intermediate 75: (2S)-2-amino-2-(4-chlorocyclohexyl)-N-(4-((S)-2-methoxy-1-((S)-2-oxo-4-(trifluoromethyl)imidazolidin-1-yl)ethyl)pyridin-2-yl)acetamide

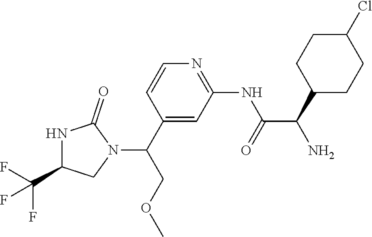 [SMILES: COCC(c1ccnc(NC(=O)[C@H](N)C2CCC(Cl)CC2)c1)N1C[C@@H](C(F)(F)F)NC1=O]

Was prepared by the synthesis route described for Int. 52, using (2S)-2-((tert-butoxycarbonyl)amino)-2-(4-chlorocyclohexyl)acetic acid, to afford (2S)-2-amino-2-(4-chlorocyclohexyl)-N-(4-((S)-2-methoxy-1-((S)-2-oxo-4-(trifluoromethyl)imidazolidin-1-yl)ethyl)pyridin-2-yl)acetamide (210 mg). LC/MS: m/z=478.2 [M+H]⁺; rt: 0.86 min (LC/MS-method B).

### Intermediate 76: (S)-2-amino-2-((1R,3s,5S)-bicyclo[3.1.0]hexan-3-yl)-N-(4-((S)-2-methoxy-1-((S)-2-oxo-4-(trifluoromethyl)imidazolidin-1-yl)ethyl)pyridin-2-yl)acetamide

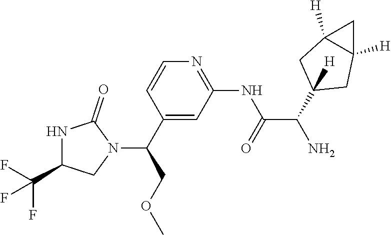

### Step 1: tert-butyl ((S)-1-((1R,3s,5S)-bicyclo[3.1.0]hexan-3-yl)-2-((4-((S)-2-methoxy-1-((S)-2-oxo-4-(trifluoromethyl)imidazolidin-1-yl)ethyl)pyridin-2-yl)amino)-2-oxoethyl)carbamate

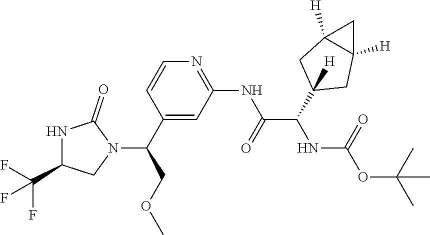

Was prepared by the synthesis route described for Int. 52, Step 1, using 2-(bicyclo[3.1.0]hexan-3-yl)-2-((tert-butoxycarbonyl)amino)acetic acid, and was purified by prep-HPLC (column: Phenomenex luna C18 (250*70 mm, 15 um); mobile phase: [water (FA)-ACN]; gradient: 33%-63% B over 18 min) to afford the product as a diastereomer mixture (1.6 g) after lyophilization. The diastereomers were separated by chiral SFC (column: DAICEL CHIRALPAK AD (250 mm*30 mm, 10 um); mobile phase: [CO₂-EtOH (0.1% NH₃H₂O)]; B %: 60%, isocratic elution mode) to afford tert-butyl ((S)-1-((1R,3s,5S)-bicyclo[3.1.0]hexan-3-yl)-2-((4-((S)-2-methoxy-1-((S)-2-oxo-4-(trifluoromethyl)imidazolidin-1-yl)ethyl)pyridin-2-yl)amino)-2-oxoethyl)carbamate (606 mg) eluting as the first diastereomer. LC/MS: m/z=542.2 [M+H]⁺; rt: 0.38 min (LC/MS-method C).

### Step 2: (S)-2-amino-2-((1R,3s,5S)-bicyclo[3.1.0]hexan-3-yl)-N-(4-((S)-2-methoxy-1-((S)-2-oxo-4-(trifluoromethyl)imidazolidin-1-yl)ethyl)pyridin-2-yl)acetamide

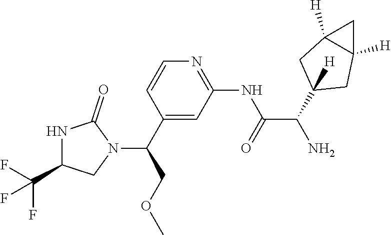

tert-butyl ((S)-1-((1R,3s,5S)-bicyclo[3.1.0]hexan-3-yl)-2-((4-((S)-2-methoxy-1-((S)-2-oxo-4-(trifluoromethyl)imidazolidin-1-yl)ethyl)pyridin-2-yl)amino)-2-oxoethyl)carbamate (150 mg) was dissolved in DCM (15 ml) and iodotrimethylsilane (111 mg) was added and the mixture stirred for 2 h at RT before addition of Na₂CO₃ solution (aq. sat., 10 ml). The mixture was stirred for 10 min before extracted with DCM (10 ml×3), dried over Na₂SO₄, filtered and evaporated under reduced pressure. LC/MS: m/z=442.2 [M+H]⁺; rt: 0.87 min (LC/MS-method B).

### Intermediate 77: (S)-2-amino-2-((1r,4S)-4-methylcyclohexyl)-N-(4-(((3S,5S)-2-oxo-5-(trifluoromethyl)pyrrolidin-3-yl)oxy)pyridin-2-yl)acetamide

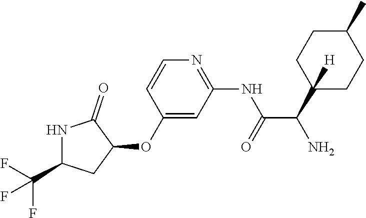

### Step 1: ethyl 2-((tert-butyldimethylsilyl)oxy)-4-oxobutanoate

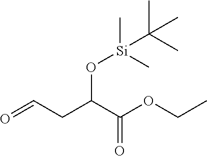 [SMILES: CCOC(=O)C(CC=O)O[Si](C)(C)C(C)(C)C]

To a solution of ethyl 2-((tert-butyldimethylsilyl)oxy)pent-4-enoate (60 g) in dioxane (2000 ml) and water (2000 ml) was added potassium osmate (IV) hydrate (2.99 g) and NalO₄ (173.81 g) and the mixture was stirred at 25° C. for 2 hr before the reaction mixture was extracted with EA (500 ml×3). The combined organic layers were washed with brine (200 ml), dried over Na₂SO₄, filtered and concentrated under reduced pressure to a residue that was purified by FC (SiO₂, Eluent of 0~5% EA/PE) to afford ethyl 2-((tert-butyldimethylsilyl)oxy)-4-oxobutanoate (20 g) as a brown liquid. ¹H NMR (400 MHz, DMSO-d6) δ ppm: 9.80 (t, J=1.6 Hz, 1H), 4.69 (t, J=5.8 Hz, 1H), 4.22 (dquin, J=3.8, 7.0 Hz, 2H), 2.89-2.80 (m, 2H), 1.32-1.28 (m, 3H), 0.89 (s, 9H), 0.13 (s, 3H), 0.09 (s, 3H).

### Step 2: ethyl (E)-2-((tert-butyldimethylsilyl)oxy)-4-(((R)-tert-butylsulfinyl)imino)butanoate

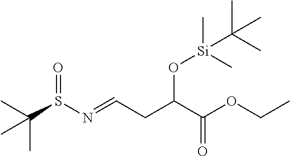 [SMILES: CCOC(=O)C(C/C=N/[S@](=O)C(C)(C)C)O[Si](C)(C)C(C)(C)C]

To a solution of ethyl 2-((tert-butyldimethylsilyl)oxy)-4-oxobutanoate (63 g) and (R)-2-methylpropane-2-sulfinamide (35.19 g) in DCM (1260 ml) was added PPTS (6.08 g) and CuSO₄ (115.84 g) and the mixture was stirred at 25° C. for 2 hr before being evaporated. The residue was purified by FC (SiO₂; Eluent of 0~30% EA/PE) to afford ethyl (E)-2-((tert-butyldimethylsilyl)oxy)-4-(((R)-tert-butylsulfinyl)imino)butanoate (82 g) as a colorless liquid. LC/MS: m/z=364.1 [M+H]⁺; rt: 0.50 min (LC/MS-method C).

### Step 3: ethyl (4S)-2-((tert-butyldimethylsilyl)oxy)-4-(((R)-tert-butylsulfinyl)amino)-5,5,5-trifluoropentanoate

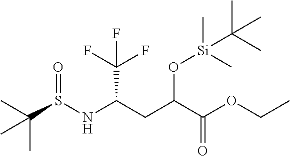 [SMILES: CCOC(=O)C(C[C@H](N[S@](=O)C(C)(C)C)C(F)(F)F)O[Si](C)(C)C(C)(C)C]

To a solution of ethyl (E)-2-((tert-butyldimethylsilyl)oxy)-4-(((R)-tert-butylsulfinyl)imino)butanoate (46 g) and tetrabutylammonium chloride (45.78 g) in THE (460 ml) was added dropwise TMSCF₃ (44.98 g) and the mixture was stirred at −5° C. for 1 hr under a nitrogen atmosphere before a solution of ammonium chloride (100 ml, aq. sat.) was added. The mixture was diluted with water (500 ml) and extracted with EA (100 ml×3) and the organic layer was washed by brine (200 ml), dried over Na₂SO₄ and evaporated. The residue was purified by FC (SiO₂; Eluent of 0~50% EA/PE) to afford ethyl (4S)-2-((tert-butyldimethylsilyl)oxy)-4-(((R)-tert-butylsulfinyl)amino)-5,5,5-trifluoropentanoate (12.1 g) as a brown liquid. LC/MS: m/z=434.2 [M+H]+; rt: 0.52 min (LC/MS-method C).

### Step 4: ethyl (4S)-4-amino-2-((tert-butyldimethylsilyl)oxy)-5,5,5-trifluoropentanoate hydrochloride

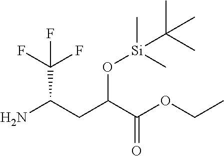 [SMILES: CCOC(=O)C(C[C@H](N)C(F)(F)F)O[Si](C)(C)C(C)(C)C]

To a solution of ethyl (4S)-2-((tert-butyldimethylsilyl)oxy)-4-(((R)-tert-butylsulfinyl)amino)-5,5,5-trifluoropentanoate (6.9 g) in DCM (138 ml) was added HCl/dioxane (4 M, 15.91 ml) and the mixture stirred at 25° C. for 4 hr before being evaporated. The crude ethyl (4S)-4-amino-2-((tert-butyldimethylsilyl)oxy)-5,5,5-trifluoropentanoate hydrochloride (6 g) was used into the next step without further purification.

### Step 5: (3R,5S)-3-((tert-butyldimethylsilyl)oxy)-5-(trifluoromethyl)pyrrolidin-2-one and (3S,5S)-3-((tert-butyldimethylsilyl)oxy)-5-(trifluoromethyl)pyrrolidin-2-one

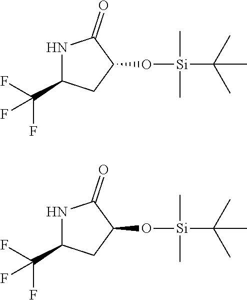 [SMILES: CC(C)(C)[Si](C)(C)O[C@@H]1C[C@@H](C(F)(F)F)NC1=O ; CC(C)(C)[Si](C)(C)O[C@H]1C[C@@H](C(F)(F)F)NC1=O]

To a solution of ethyl (4S)-4-amino-2-((tert-butyldimethylsilyl)oxy)-5,5,5-trifluoropentanoate hydrochloride (6 g) in THE (55 ml) was added LDA (2 M, 24.60 ml) dropwise at −78° C. and the mixture was stirred at this temperature for 2 hr before addition of saturated ammonium chloride solution (30 ml). The mixture was extracted with EA (20 ml×3), dried over Na₂SO₄ and concentrated. The residue was purified by FC (SiO₂; Eluent of 0~80% EA/PE) to afford (3R,5S)-3-((tert-butyldimethylsilyl)oxy)-5-(trifluoromethyl)pyrrolidin-2-one (DS1; 1.2 g) as a colorless solid and (3S,5S)-3-((tert-butyldimethylsilyl)oxy)-5-(trifluoromethyl)pyrrolidin-2-one (DS2; 0.99 g) as a yellow solid.

DS1: ¹H NMR (400 MHz, CDCl₃) δ ppm: 5.87-5.72 (m, 1H), 4.33 (t, J=7.8 Hz, 1H), 3.99 (qd, J=6.6, 13.4 Hz, 1H), 2.64 (td, J=8.2, 13.4 Hz, 1H), 2.16-2.03 (m, 1H), 0.95-0.88 (m, 9H), 0.17 (d, J=10.8 Hz, 5H). DS2: ¹H NMR (400 MHz, CDCl₃) δ ppm: 5.82 (d, J=3.0 Hz, 1H), 4.33 (t, J=7.8 Hz, 1H), 4.03-3.94 (m, 1H), 2.64 (td, J=8.0, 13.4 Hz, 1H), 2.14-2.06 (m, 1H), 0.92 (s, 9H), 0.16-0.14 (m, 3H), 0.11 (s, 3H).

### Step 6: (3S,5S)-3-hydroxy-5-(trifluoromethyl)pyrrolidin-2-one

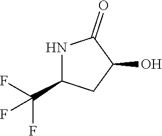 [SMILES: O=C1N[C@H](C(F)(F)F)C[C@@H]1O]

To a solution of (3S,5S)-3-((tert-butyldimethylsilyl)oxy)-5-(trifluoromethyl)-pyrrolidin-2-one DS2 (1.5 g) in THE (15 ml) was added triethylamine trihydrofluoride (14.84 g) and the mixture was stirred at 20° C. for 6 hr before the reaction mixture was quenched by addition of water (100 ml) at 20° C., and extracted with EA (30 ml×3). The aqueous layer was adjusted to pH 8 by addition of NaHCO₃ solution (aq. sat.) and extracted with EA (30 ml×3) and the organic layer was dried over anhydrous Na₂SO₄, filtered and concentrated under reduced pressure to give a residue that was purified by FC (SiO₂; Eluent of 0~100% EA/PE). This afforded (3S,5S)-3-hydroxy-5-(trifluoromethyl)pyrrolidin-2-one (561 mg) as a colorless solid. ¹H NMR (400 MHz, CDCl₃) δ ppm: δ=6.25 (br d, J=7.0 Hz, 1H), 4.40 (t, J=8.5 Hz, 1H), 4.16-4.01 (m, 1H), 2.76 (td, J=8.2, 13.4 Hz, 1H), 2.19-2.09 (m, 1H).

### Step 6B: (3R,5S)-3-hydroxy-5-(trifluoromethyl)pyrrolidin-2-one

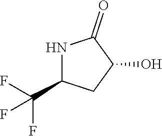 [SMILES: O=C1N[C@H](C(F)(F)F)C[C@H]1O]

Prepared by the procedure described for (3S,5S)-3-hydroxy-5-(trifluoromethyl)pyrrolidin-2-one (Step 6), to afford (3R,5S)-3-hydroxy-5-(trifluoromethyl)pyrrolidin-2-one as a colorless solid. ¹H NMR (400 MHz, CDCl₃) δ ppm: δ=7.16 (br s, 1H), 4.51 (t, J=8.6 Hz, 1H), 4.16-4.04 (m, 1H), 2.71-2.61 (m, 1H), 2.33 (td, J=9.0, 14.1 Hz, 1H).

### Step 8: tert-butyl ((S)-2-((4-(benzyloxy)pyridin-2-yl)amino)-1-((1r,4S)-4-methylcyclohexyl)-2-oxoethyl)carbamate

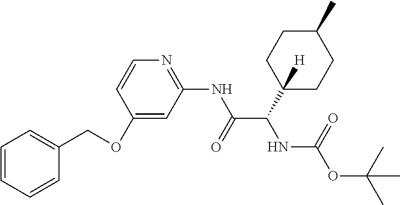

To a solution of (S)-2-((tert-butoxycarbonyl)amino)-2-((1r,4S)-4-methylcyclohexyl)acetic acid (5 g) and 4-(benzyloxy)pyridin-2-amine (4.80 g) in DCM (100 ml) was added EDCl (5.30 g) and DMAP (450 mg) and the mixture was stirred at 25° C. for 12 hr before being diluted 1M HCl (5 m.). After 10 min water (300 ml) was added and the mixture extracted with EA (100 ml×3). The combined organic layers were washed with brine (200 ml), dried over Na₂SO₄, filtered and concentrated under reduced pressure to give a residue that was purified by FC (SiO₂, PE/EA=1/0 to 2/1) to afford tert-butyl ((S)-2-((4-(benzyloxy)pyridin-2-yl)amino)-1-((1r,4S)-4-methylcyclohexyl)-2-oxoethyl)carbamate (7.5 g) as a colorless solid. LC/MS: m/z=454.3 [M+H]⁺; rt: 0.47 min (LC/MS-method C).

### Step 9: tert-butyl ((S)-2-((4-hydroxypyridin-2-yl)amino)-1-((1r,4S)-4-methylcyclohexyl)-2-oxoethyl)carbamate

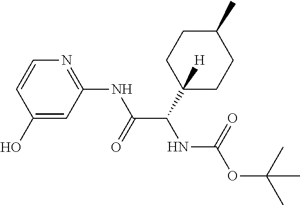

To a solution of tert-butyl ((S)-2-((4-(benzyloxy)pyridin-2-yl)amino)-1-((1r,4S)-4-methylcyclohexyl)-2-oxoethyl)carbamate (5 g) in MeOH (100 ml), was added Pd/C (1 g, 10%) under a nitrogen atmosphere. The suspension was degassed and purged with hydrogen 3 times and the mixture was stirred under hydrogen (15 Psi) at 25° C. for 12 hr before the reaction mixture was filtered and concentrated under reduced pressure to a residue that purified by SFC (column: DAICEL CHIRALPAK IC (250 mm*30 mm, 10 um); mobile phase: [CO₂-EtOH (0.1% NH₃H₂O)]; B %: 35%-35%, 2.9 min). This afforded tert-butyl ((S)-2-((4-hydroxypyridin-2-yl)amino)-1-((1r,4S)-4-methylcyclohexyl)-2-oxoethyl)carbamate (1.4 g) as a colorless solid. LC/MS: m/z=364.3 [M+H]⁺; rt: 0.49 min (LC/MS-method C).

### Step 10: tert-butyl ((S)-1-((1r,4S)-4-methylcyclohexyl)-2-oxo-2-((4-(((3S,5S)-2-oxo-5-(trifluoromethyl)pyrrolidin-3-yl)oxy)pyridin-2-yl)amino)ethyl)carbamate

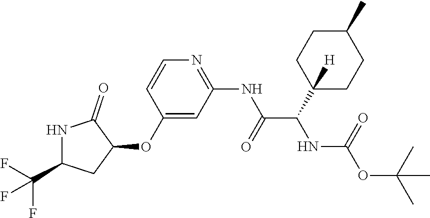

To a solution of tert-butyl ((S)-2-((4-hydroxypyridin-2-yl)amino)-1-((1r,4S)-4-methylcyclohexyl)-2-oxoethyl)carbamate (Step 9; 1.25 g) in THE (25 ml) was added PPh₃ (1.35 g) and DIAD (1.00 mL) at 0° C. under a nitrogen atmosphere and stirred for 30 min before (3S,5S)-3-hydroxy-5-(trifluoromethyl)pyrrolidin-2-one (Step 6; 697.90 mg) was added. The mixture was stirred at 20° C. for 12 hr before water (50 ml) was added and extracted with EA (20 ml×3) and the combined organic layers were washed with brine (30 ml), dried over Na₂SO₄, filtered and concentrated under reduced pressure. The residue was purified by prep-HPLC (column: Phenomenex luna C18 250*80 mm*10 um; mobile phase: [water (0.1% FA)-ACN]; gradient: 40%-70% B over 25 min) to afford tert-butyl ((S)-1-((1r,4S)-4-methylcyclohexyl)-2-oxo-2-((4-(((3S,5S)-2-oxo-5-(trifluoromethyl)pyrrolidin-3-yl)oxy)pyridin-2-yl)amino)ethyl)carbamate (790 mg) as a colorless solid.

### Step 11: (S)-2-amino-2-((1r,4S)-4-methylcyclohexyl)-N-(4-(((3S,5S)-2-oxo-5-(trifluoromethyl)pyrrolidin-3-yl)oxy)pyridin-2-yl)acetamide

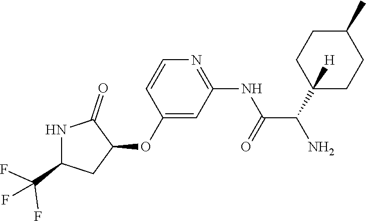

Following the procedure described for Int. 46, using tert-butyl ((S)-1-((1r,4S)-4-methylcyclohexyl)-2-oxo-2-((4-(((3S,5S)-2-oxo-5-(trifluoromethyl)pyrrolidin-3-yl)oxy)pyridin-2-yl)amino)ethyl)carbamate (386 mg), afforded (S)-2-amino-2-((1r,4S)-4-methylcyclohexyl)-N-(4-(((3S,5S)-2-oxo-5-(trifluoromethyl)pyrrolidin-3-yl)oxy)pyridin-2-yl)acetamide-076 (250 mg). LC/MS: m/z=415.1 [M+H]⁺; rt: 1.26 min (LC/MS-method A).

### Intermediate 78: (S)-2-amino-2-((1r,4S)-4-methylcyclohexyl)-N-(4-(((3R,5S)-2-oxo-5-(trifluoromethyl)pyrrolidin-3-yl)oxy)pyridin-2-yl)acetamide

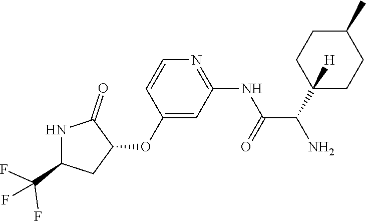

The title compound was prepared by the procedure described for Int. 77, using (3R,5S)-3-hydroxy-5-(trifluoromethyl)pyrrolidin-2-one (Step 6B). LC/MS: m/z=415.1 [M+H]⁺; rt: 1.29 min (LC/MS-method A).

### Intermediate 79: (2S)-2-amino-2-(4,4-difluorocyclohexyl)-N-(4-((2-oxo-5-(trifluoromethyl)-2,5-dihydro-1H-pyrrol-3-yl)methyl)pyridin-2-yl)acetamide

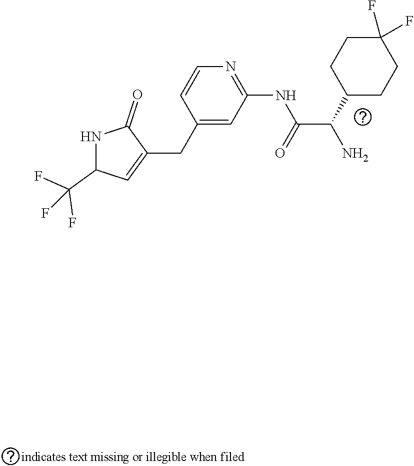 [SMILES: N[C@H](C(=O)Nc1cc(CC2=CC(C(F)(F)F)NC2=O)ccn1)C1CCC(F)(F)CC1]

### Step 1: methyl 2-oxo-5-(trifluoromethyl)tetrahydrofuran-3-carboxylate

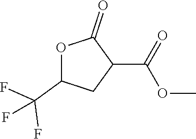

To a solution of dimethyl malonate (8.67 ml) in THE (50 ml) was added dropwise LiHMDS (1 M, 98.40 ml) at 0° C. and the mixture stirred at 0° C. for 1 h before 2-(trifluoromethyl)oxirane (16.96 g) was added at 0° C. The reaction mixture was stirred at 25° C. for 12 h under a nitrogen atmosphere before ammonium chloride solution (sat·aq. 100 ml) was added mixture extracted with EA (50 ml). The organic layer was dried over Na₂SO₄, filtered and concentrated in vacuo to afford a residue, which was purified by FC (SiO₂, PE/EA=5/1 to 3/1) to afford methyl 2-oxo-5-(trifluoromethyl)tetrahydrofuran-3-carboxylate (7 g) as a light yellow oil. ¹H NMR (400 MHz, CDCl₃) δ ppm: 4.69-4.99 (m, 1H), 3.86 (d, J=2.25 Hz, 3H), 3.72-3.79 (m, 1H), 2.82-3.01 (m, 1H), 2.55-2.79 (m, 1H).

### Step 2: methyl 2-((2-((S)-2-((tert-butoxycarbonyl)amino)-2-(4,4-difluorocyclohexyl)acetamido)pyridin-4-yl)methyl)-5,5,5-trifluoro-4-hydroxypentanoate

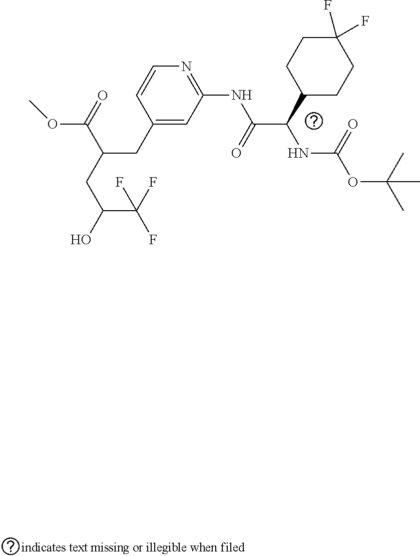 [SMILES: COC(=O)C(Cc1ccnc(NC(=O)[C@H](NC(=O)OC(C)(C)C)C2CCC(F)(F)CC2)c1)CC(O)C(F)(F)F]

To a mixture of methyl 2-oxo-5-(trifluoromethyl)tetrahydrofuran-3-carboxylate (Step 1; 800 mg), tert-butyl (S)-(2-((4-(bromomethyl)pyridin-2-yl)amino)-1-(4,4-difluorocyclohexyl)-2-oxoethyl)carbamate (Int. 11; 408 mg) and K₂CO₃ (479 mg) in DMF (20 ml) was added KI (58 mg) at 25° C. and the mixture stirred at 25° C. for 3 h. MeOH (10 ml) was added and stirring continued at 25° C. for 1 h and 60° C. for 1 h. Brine (60 ml) was added and the mixture extracted with EA (60 ml) and the organic layer was washed with brine (50 ml×2), dried over Na₂SO₄, filtered and concentrated in vacuo to give a residue, which was purified by FC (SiO₂, PE/EA=5/1 to 3/1) to afford methyl 2-((2-((S)-2-((tert-butoxycarbonyl)amino)-2-(4,4-difluorocyclohexyl)acetamido)pyridin-4-yl)methyl)-5,5,5-trifluoro-4-hydroxypentanoate (580 mg) as a white solid, which was used directly in the next step. LC/MS: m/z=568.4 [M+H]⁺; rt: 0.38 min (LC/MS-method C).

### Step 3: methyl 2-((2-((S)-2-((tert-butoxycarbonyl)amino)-2-(4,4-difluorocyclohexyl)-acetamido)pyridin-4-yl)methyl)-5,5,5-trifluoro-4,4-dihydroxypentanoate

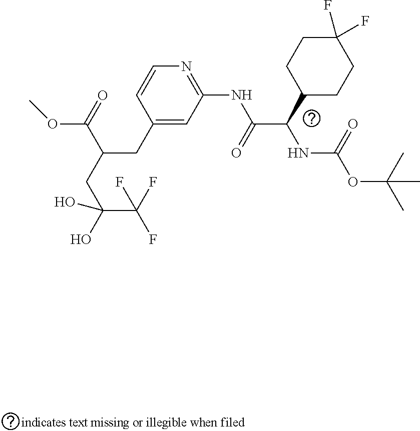 [SMILES: COC(=O)C(Cc1ccnc(NC(=O)[C@H](NC(=O)OC(C)(C)C)C2CCC(F)(F)CC2)c1)CC(O)(O)C(F)(F)F]

To a solution of methyl 2-((2-((S)-2-((tert-butoxycarbonyl)amino)-2-(4,4-difluorocyclohexyl)acetamido)pyridin-4-yl)methyl)-5,5,5-trifluoro-4-hydroxypentanoate (580 mg) in DCM (10 ml) was added DMP (867 mg) at 0° C. and then stirred at 25° C. for 3 h. The pH was adjusted to 9 using Na₂CO₃ (sat·aq.) and then extracted with DCM (30 ml) and EA (30 ml). The combined organic layers were dried over Na₂SO₄, filtered and concentrated in vacuo to give a residue, which was purified by column chromatography (SiO₂, PE/EA=3/1 to 1/1) to afford methyl 2-((2-((S)-2-((tert-butoxycarbonyl)amino)-2-(4,4-difluorocyclohexyl)acetamido)pyridin-4-yl)methyl)-5,5,5-trifluoro-4,4-dihydroxypentanoate (500 mg) as a brown solid. LC/MS: m/z=584.3 [M+H]⁺; rt: 0.44 min (LC/MS-method C).

### Step 4: tert-butyl ((1S)-1-(4,4-difluorocyclohexyl)-2-((4-((5-hydroxy-2-oxo-5-(trifluoromethyl)pyrrolidin-3-yl)methyl)pyridin-2-yl)amino)-2-oxoethyl)carbamate

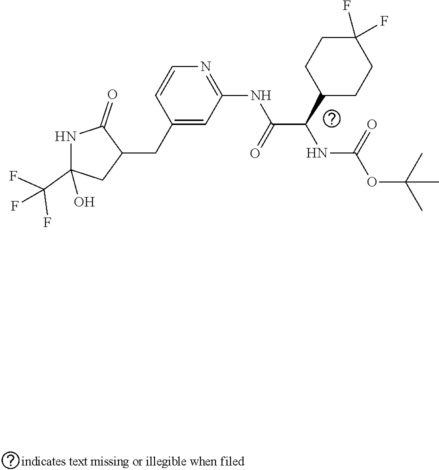 [SMILES: CC(C)(C)OC(=O)N[C@@H](C(=O)Nc1cc(CC2CC(O)(C(F)(F)F)NC2=O)ccn1)C1CCC(F)(F)CC1]

To a solution of methyl 2-((2-((S)-2-((tert-butoxycarbonyl)amino)-2-(4,4-difluorocyclohexyl)acetamido)pyridin-4-yl)methyl)-5,5,5-trifluoro-4,4-dihydroxypentanoate (500 mg) in toluene (5 ml) was added AcOH (150 μl, 3 eq) and NH₄OAc (198 mg) and then the mixture was stirred at 65° C. for 12 h. The pH was adjusted to 9 with NaHCO₃ (sat·aq.) and then extracted with DCM (10 ml) and EA (10 ml). The combined organic layers were dried over Na₂SO₄, filtered and concentrated in vacuo to give a residue which was purified by column (SiO₂, PE/EA=1/1) to afford tert-butyl ((1S)-1-(4,4-difluorocyclohexyl)-2-((4-((5-hydroxy-2-oxo-5-(trifluoromethyl)pyrrolidin-3-yl)methyl)pyridin-2-yl)amino)-2-oxoethyl)carbamate (395 mg) as a colorless oil. LC/MS: m/z=551.3 [M+H]⁺; rt: 0.42 min (LC/MS-method C).

### Step 5: tert-butyl ((1S)-1-(4,4-difluorocyclohexyl)-2-oxo-2-((4-((2-oxo-5-(trifluoromethyl)-2,5-dihydro-1H-pyrrol-3-yl)methyl)pyridin-2-yl)amino)ethyl)carbamate

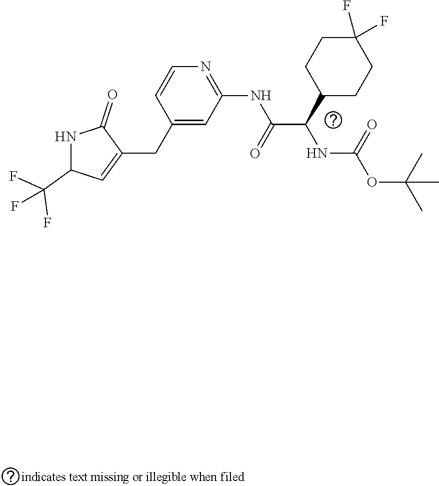 [SMILES: CC(C)(C)OC(=O)N[C@@H](C(=O)Nc1cc(CC2=CC(C(F)(F)F)NC2=O)ccn1)C1CCC(F)(F)CC1]

To a solution of tert-butyl ((1S)-1-(4,4-difluorocyclohexyl)-2-((4-((5-hydroxy-2-oxo-5-(trifluoromethyl)pyrrolidin-3-yl)methyl)pyridin-2-yl)amino)-2-oxoethyl)carbamate (250 mg) in pyridine (1.25 ml) was added trifluoroacetic anhydride (158 μl) at 0° C. and then the mixture was stirred at 20° C. for 3 h. Brine (4 ml) was added and the mixture extracted with DCM (4 ml×2). The combined organic layers were dried over Na₂SO₄, filtered and concentrated in vacuo to give a residue which was purified by FC (SiO₂, PE/EA=5/1 to 1/1) to afford tert-butyl ((1S)-1-(4,4-difluorocyclohexyl)-2-oxo-2-((4-((2-oxo-5-(trifluoromethyl)-2,5-dihydro-1H-pyrrol-3-yl)methyl)pyridin-2-yl)amino)ethyl)carbamate I-078-5 (225 mg) as a colorless oil. LC/MS: m/z=533.3 [M+H]⁺; rt: 0.43 min (LC/MS-method C).

### Step 6: (2S)-2-amino-2-(4,4-difluorocyclohexyl)-N-(4-((2-oxo-5-(trifluoromethyl)-2,5-dihydro-1H-pyrrol-3-yl)methyl)pyridin-2-yl)acetamide

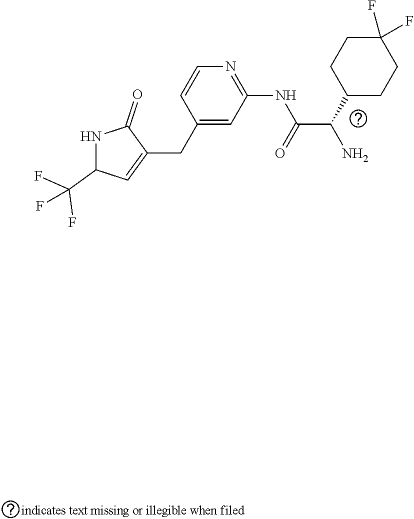 [SMILES: N[C@H](C(=O)Nc1cc(CC2=CC(C(F)(F)F)NC2=O)ccn1)C1CCC(F)(F)CC1]

Following the procedure described for Int. 46, using tert-butyl ((1S)-1-(4,4-difluorocyclohexyl)-2-oxo-2-((4-((2-oxo-5-(trifluoromethyl)-2,5-dihydro-1H-pyrrol-3-yl)methyl)pyridin-2-yl)amino)ethyl)carbamate (220 mg), afforded (2S)-2-amino-2-(4,4-difluorocyclohexyl)-N-(4-((2-oxo-5-(trifluoromethyl)-2,5-dihydro-1H-pyrrol-3-yl)methyl)pyridin-2-yl)acetamide (160 mg). LC/MS: m/z=433.2 [M+H]⁺; rt: 1.12 min (LC/MS-method A).

### EXAMPLE COMPOUNDS

### Example 1: Tert-butyl ((S)-1-((1r,4S)-4-methylcyclohexyl)-2-oxo-2-((4-(((S)-2-oxo-4-(trifluoromethyl)imidazolidin-1-yl)methyl)pyridin-2-yl)amino)ethyl)carbamate

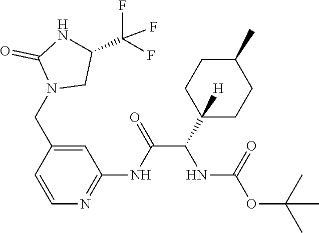

Tert-butyl ((S)-2-((4-((((S)-2-amino-3,3,3-trifluoropropyl)amino)methyl)pyridin-2-yl)amino)-1-((1r,4S)-4-methylcyclohexyl)-2-oxoethyl)carbamate (Intermediate 1; 176 mg) was dissolved in THE (12 ml). After the addition of DIPEA (0.22 ml) the mixture was heated to 65° C. for 5 min. Then CDI (205 mg) was added, and stirring was continued overnight at 65° C. At RT the mixture was concentrated, and saturated NaHCO₃ solution and DCM were added. The organic layer was separated, and the aqueous phase was extracted twice with DCM. The combined organic phases were dried over sodium sulphate, filtered and concentrated in vacuo. The residue was purified by silica gel column chromatography (12 g silica gel; gradient: DCM/EtOH: 100% DCM for 5 min, 100% to 92% DCM in 60 min; 92% DCM for 10 min), and the combined product containing fractions were concentrated in vacuo. The residue was further purified by preparative RP HPLC (Agilent Prep C18 (10 μm, 21.2 mm×250 mm); 50 ml/min, from 90% H₂O/10% ACN to 90% ACN/10% H₂O in 12.5 min). The pure product containing fractions were combined, the ACN was partly removed, and the residue freeze dried to yield 133 mg of the title compound. LC/MS: m/z=514.2 [M+H]⁺; rt: 2.32 min (LC/MS-method A). ¹H NMR (400 MHz, DMSO-de) δ ppm: 10.34 (s, 1H), 8.27 (d, J=5.2 Hz, 1H), 7.97 (s, 1H), 7.65 (d, J=2.0 Hz, 1H), 6.93 (dd, J=5.2 Hz, 1.2 Hz, 1H), 6.91 (d, overlapped, 1H), 4.40 (m, 1H), 4.39 (d, J=16.2 Hz, 1H),), 4.21 (d, J=16.2 Hz, 1H), 4.05 (m, 1H), 3.58 (dd, J=10.0 Hz, 10.0 Hz, 1H), ~3.30 (dd, signal below water signal, 1H), 1.71-1.49 (m, 5H), 1.37 (s, 9H), 1.32-0.99 (m, 3H), 0.86-0.77 (m, 2H), 0.83 (d, J=6.5 Hz, 3H).

### Example 2: 4-Cyclopropyl-N—((S)-1-((1r,4S)-4-methylcyclohexyl)-2-oxo-2-((4-(((S)-2-oxo-4-(trifluoromethyl)imidazolidin-1-yl)methyl)pyridin-2-yl)amino)ethyl)-1,2,5-oxadiazole-3-carboxamide

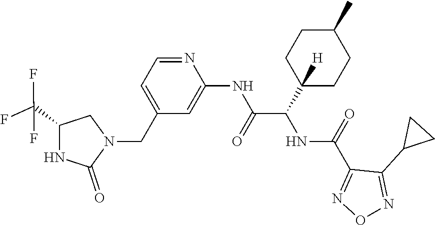

To a mixture of (S)-2-amino-2-((1r,4S)-4-methylcyclohexyl)-N-(4-(((S)-2-oxo-4-(trifluoromethyl)-imidazolidin-1-yl)methyl)pyridin-2-yl)acetamide HCl salt (Intermediate 2; 20 mg), 4-cyclopropyl-1,2,5-oxadiazole-3-carboxylic acid (7 mg) and DCM (1.5 ml) DIPEA (36 μl) and T3P (73 μl) were added. After 0.5 h, the mixture was concentrated in vacuo. The residue was purified by preparative RP HPLC (Purosphere® STAR-RP18, 25 mm×250 mm, 10 μm; 30 ml/min, from 95% H₂O/5% ACN to 10% H₂O/90% ACN in 45 min). The pure product containing fractions were combined, the ACN was partly removed, and the residue freeze dried to yield 15 mg of the title compound. LC/MS: m/z=550.12 [M+H]⁺; rt: 2.42 min (LC/MS-method A). ¹H NMR (400 MHz, DMSO-d₆) δ ppm: 10.72 (s, 1H), 9.16 (d, J=8.1 Hz, 1H), 8.29 (d, J=5.2 Hz, 1H), 7.98 (s, 1H), 7.65 (d, J=2.0 Hz, 1H), 6.95 (dd, J=5.2 Hz, 1.2 Hz, 1H), 4.62 (dd, J=8.1 Hz, 7.6 Hz, 1H), 4.41 (m, 1H), 4.39 (d, J=16.2 Hz, 1H),), 4.22 (d, J=16.2 Hz, 1H), 3.59 (dd, J=10.0 Hz, 10.0 Hz, 1H), ~3.30 (dd, signal below water signal, 1H), 2.27 (m, 1H), 1.83-1.58 (m, 5H), 1.29-0.94 (m, 7H), 0.91-0.82 (m, 2H), 0.85 (d, J=6.5 Hz, 3H).

### Example 3: 1-Methyl-N—((S)-1-((1r,4S)-4-methylcyclohexyl)-2-oxo-2-((4-(((S)-2-oxo-4-(trifluoromethyl)imidazolidin-1-yl)methyl)pyridin-2-yl)amino)ethyl)-1H-pyrazole-5-carboxamide

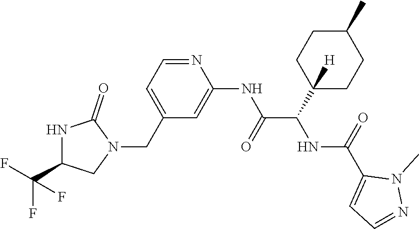

Following the procedure of Example 2, 2-methylpyrazole-3-carboxylic acid (50 mg) was reacted with the amine (160 mg). To complete the reaction (reaction monitored by LC/MS), additional DIPEA (76 μl) and T3P (228 μl) were added after 2 h. After 1 further h, the mixture was concentrated in vacuo. The residue was purified by preparative RP HPLC (Purosphere® STAR-RP18, 25 mm×250 mm, 10 μm; 30 ml/min, from 95% H₂O/5% ACN to 10% H₂O/90% ACN in 45 min). The pure product containing fractions were combined, the ACN was partly removed, and the residue freeze dried to yield 108 mg of the title compound. LC/MS: m/z=522.2 [M+H]⁺; rt: 1.98 min (LC/MS-method A). ¹H NMR (400 MHz, DMSO-de) δ ppm: 10.60 (s, 1H), 8.43 (d, J=8.0 Hz, 1H), 8.28 (d, J=5.2 Hz, 1H), 7.98 (s, 1H), 7.64 (d, J=2.0 Hz, 1H), 7.46 (d, J=2.0 Hz, 1H), 7.03 (d, J=2.0 Hz, 1H), 6.94 (dd, J=5.2 Hz, 1.2 Hz, 1H), 4.51 (dd, J=8.5 Hz, 8.0 Hz, 1H), 4.40 (m, 1H), 4.38 (d, J=16.2 Hz, 1H),), 4.21 (d, J=16.2 Hz, 1H), 4.01 (s, 3H), 3.58 (dd, J=10.0 Hz, 10.0 Hz, 1H), 3.29 (dd, signal below water signal, 1H), 2.27 (m, 1H), 1.84-1.55 (m, 5H), 1.29-1.19 (m, 2H), 1.06 (m, 1H), 0.92-0.81 (m, 2H), 0.85 (d, J=6.5 Hz, 3H).

### Example 4: 1-isopropyl-N—((S)-1-((1r,4S)-4-methylcyclohexyl)-2-oxo-2-((4-(((S)-2-oxo-4-(trifluoromethyl)imidazolidin-1-yl)methyl)pyridin-2-yl)amino)ethyl)-1H-pyrazole-5-carboxamide

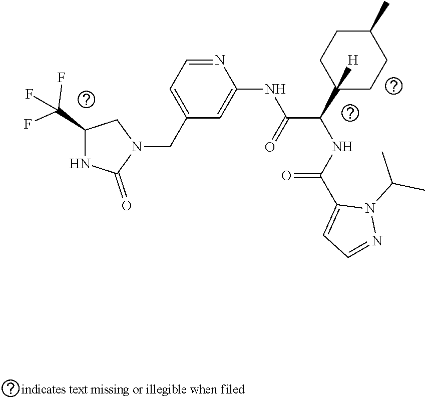

Example 4 was prepared analogously to Example 2 using 2-isopropylpyrazole-3-carboxylic acid to give the title compound in an 11.7 mg (53%) yield. LC/MS: m/z=550.2 [M+H]⁺; rt: 2.21 min (LC/MS-method A). ¹H NMR (400 MHz, DMSO-de) δ ppm: 10.58 (s, 1H), 8.42 (d, J=8.0 Hz, 1H), 8.28 (d, J=5.2 Hz, 1H), 7.98 (s, 1H), 7.64 (d, J=2.0 Hz, 1H), 7.49 (d, J=1.9 Hz, 1H), 6.94 (dd, J=5.2 Hz, 1.2 Hz, 1H), 6.93 (d, J=1.9 Hz, 1H), 5.37 (m, 1H), 4.49 (dd, J=8.4 Hz, 8.0 Hz, 1H), 4.41 (m, 1H), 4.39 (d, J=16.2 Hz, 1H),), 4.21 (d, J=16.2 Hz, 1H), 3.58 (dd, J=10.0 Hz, 10.0 Hz, 1H), 3.29 (dd, signal below water signal, 1H), 1.84-1.56 (m, 5H), 1.35 (d, J=6.6 Hz, 3H), 1.33 (d, J=6.6 Hz, 3H), 1.31-1.18 (m, 2H), 1.07 (m, 1H), 0.91-0.81 (m, 2H), 0.85 (d, J=6.5 Hz, 3H).

### Example 5: Benzyl ((S)-1-((1r,4S)-4-methylcyclohexyl)-2-oxo-2-((4-(((S)-2-oxo-4-(trifluoromethyl)imidazolidin-1-yl)methyl)pyridin-2-yl)amino)ethyl)carbamate

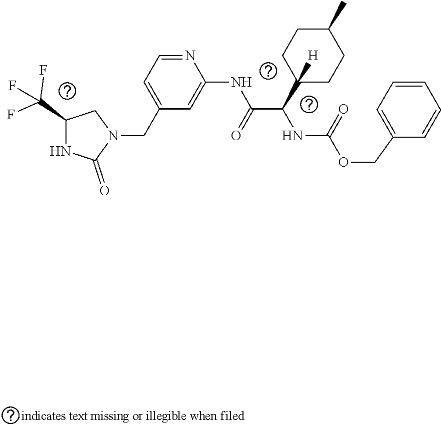

(S)-2-Amino-2-((1r,4S)-4-methylcyclohexyl)-N-(4-(((S)-2-oxo-4-(trifluoromethyl)-imidazolidin-1-yl)methyl)pyridin-2-yl)acetamide HCl salt (Intermediate 2; 18 mg) was dissolved in dry DCM (1 ml) and with stirring DIPEA (24 μl) was added followed by benzyl chloroformate (7 μl, dissolved in 0.5 ml DCM). After 15 min, the mixture was poured into water, and the aqueous phase was extracted with DCM (3×). The combined organic phases were dried over sodium sulphate, filtered, and concentrated in vacuo. The residue was purified by preparative RP HPLC (Purosphere® STAR-RP18, 25 mm×250 mm, 10 μm; 30 ml/min, from 95% H₂O/5% ACN to 10% H₂O/90% ACN in 45 min). The pure product containing fractions were combined, the ACN was partly removed, and the residue freeze dried to yield 6.7 mg of the title compound. LC/MS: m/z=548.2 [M+H]⁺; rt: 2.35 min (LC/MS-method A). ¹H NMR (400 MHz, DMSO-d₆) δ ppm: 10.45 (s, 1H), 8.27 (d, J=5.2 Hz, 1H), 7.98 (s, 1H), 7.65 (d, J=2.0 Hz, 1H), 7.46 (d, J=8.3 Hz, 1H), 7.36-7.29 (m, 5H), 6.93 (dd, J=5.2 Hz, 1.2 Hz, 1H), 5.02 (s, 2H), 4.41 (m, 1H), 4.38 (d, J=16.2 Hz, 1H),), 4.22 (d, J=16.2 Hz, 1H), 4.15 (dd, J=8.3 Hz, 7.5 Hz, 1H), 3.58 (dd, J=10.0 Hz, 10.0 Hz, 1H), ~3.30 (dd, signal below water signal, 1H), 1.73-1.49 (m, 5H), 1.35 (d, J=6.6 Hz, 3H), 1.33 (d, J=6.6 Hz, 3H), 1.26-1.14 (m, 2H), 1.06 (m, 1H), 0.87-0.78 (m, 2H), 0.83 (d, J=6.5 Hz, 3H).

### Example 6: Tert-butyl ((S)-1-(4,4-difluorocyclohexyl)-2-oxo-2-((4-(((S)-2-oxo-4-(trifluoromethyl)imidazolidin-1-yl)methyl)pyridin-2-yl)amino)ethyl)carbamate

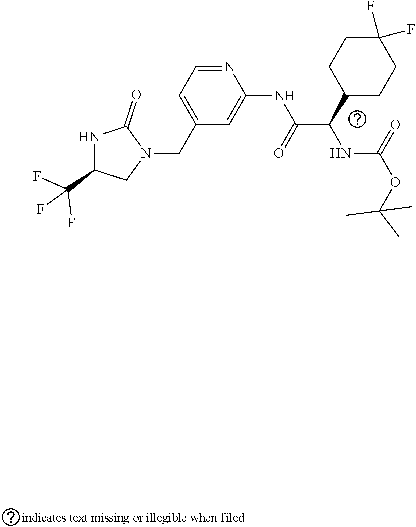 [SMILES: CC(C)(C)OC(=O)N[C@@H](C(=O)Nc1cc(CN2C[C@@H](C(F)(F)F)NC2=O)ccn1)C1CCC(F)(F)CC1]

Tert-butyl ((S)-2-((4-((((S)-2-amino-3,3,3-trifluoropropyl)amino)methyl)pyridin-2-yl)amino)-1-(4,4-difluorocyclohexyl)-2-oxoethyl)carbamate TFA salt (Intermediate 3; 549 mg) was dissolved in dry THE (15 ml) and DIPEA (613 at μl) was added under Ar at rt. After heating for 5 min at 65° C. CDI (162 mg) was added. After stirring for 6 h at 65° C. and standing overnight, heating at 65° C. was continued for 8 h and additional CDI (131 mg) was added. After standing overnight again the mixture was concentrated, saturated NaHCO₃ solution was added and the aqueous mixture was extracted with DCM (3×). The combined organic phases were dried over sodium sulphate, filtered and concentrated in vacuo. The residue was purified by preparative RP HPLC (Purosphere® STAR-RP18, 25 mm×250 mm, 10 μm; 30 ml/min, from 95% H₂O/5% ACN to 10% H₂O/90% ACN in 45 min). The pure product containing fractions were combined, the ACN was partly removed, and the residue freeze dried to yield 291 mg of the title compound. LC/MS: m/z=536.2 [M+H]⁺; rt: 2.04 min (LC/MS-method A). ¹H NMR (400 MHz, DMSO-d₆) δ ppm: 10.46 (s, 1H), 8.28 (d, J=5.2 Hz, 1H), 7.98 (s, 1H), 7.65 (d, J=2.0 Hz, 1H), 7.07 (d, J=8.2 Hz, 1H), 6.94 (dd, J=5.2 Hz, 1.1 Hz, 1H), 4.43 (m, 1H), 4.39 (d, J=16.2 Hz, 1H), 4.22 (d, J=16.2 Hz, 1H), 4.19 (dd, overlapped, 1H), 3.58 (dd, J=10.0 Hz, 10.0 Hz, 1H), ~3.30 (dd, 1H, signal below water signal), 2.01 (m, 2H), 1.81-1.59 (m, 5H), 1.43-1.27 (m, 2H), 1.38 (s, 9H).

### Example 7: Tert-butyl 6-(((S)-1-((1r,4S)-4-methylcyclohexyl)-2-oxo-2-((4-(((S)-2-oxo-4-(trifluoromethyl)imidazolidin-1-yl)methyl)pyridin-2-yl)amino)ethyl)-carbamoyl)-3,4-dihydropyrrolo[1,2-a]pyrazine-2(1H)-carboxylate

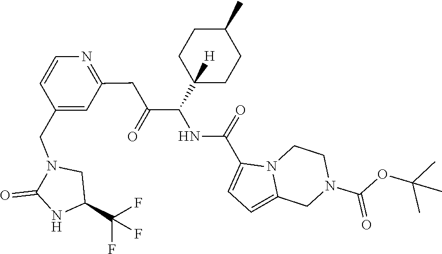

2-Tert-butoxycarbonyl-3,4-dihydro-1H-pyrrolo[1,2-a]pyrazine-6-carboxylic acid (20 mg) was dissolved in dry DMF (1.5 ml), and DIPEA (39 μl) and HATU (25 mg) were added with stirring. After 30 minutes (S)-2-amino-2-((1r,4S)-4-methylcyclohexyl)-N-(4-(((S)-2-oxo-4-(trifluoromethyl)imidazolidin-1-yl)methyl)pyridin-2-yl)acetamide HCl salt (Intermediate 2; 20 mg) was added. After stirring overnight the mixture was poured onto brine, and the aqueous mixture was extracted with EA (2×). The combined organic phases were dried over sodium sulphate, filtered and concentrated in vacuo. The residue was purified by preparative RP HPLC (Purosphere® STAR-RP18, 25 mm×250 mm, 10 μm; 30 ml/min, from 95% H₂O/5% ACN to 10% H₂O/90% ACN in 45 min). The pure product containing fractions were combined, the ACN was partly removed, and the residue freeze dried. After further purification by silica gel column chromatography (4 g silica gel; gradient: DCM/EtOH: 100% DCM for 5 min, 100% to 90% DCM in 30 min; 90% DCM for 10 min), combining the product containing fractions and concentrating them in vacuo, the residue was freeze dried from water/ACN to yield 10 mg of the title compound. LC/MS: m/z=662.2 [M+H]⁺; rt: 2.56 min (LC/MS-method A). ¹H NMR (400 MHz, DMSO-d₆) δ ppm: 10.44 (s, 1H), 8.27 (d, J=5.2 Hz, 1H), 7.98 (s, 1H), 7.85 (d, J=8.3 Hz, 1H), 7.64 (d, J=2.0 Hz, 1H), 6.99 (d, J=3.9 Hz, 1H), 6.93 (dd, J=5.2 Hz, 1.1 Hz, 1H), 5.96 (d, J=3.9 Hz, 1H), 4.53 (s, 2H), 4.44-4.19 (m, 6H), 3.68-3.55 (m, 3H), ~3.30 (dd, 1H, signal below water signal), 1.84-1.55 (m, 5H), 1.42 (s, 9H), 1.31-1.15 (m, 2H), 1.03 (m, 1H), 0.91-0.81 (m, 2H), 0.85 (d, J=6.5 Hz, 3H).

Analogously to Example 2, using (S)-2-amino-2-(4,4-difluorocyclohexyl)-N-(4-(((S)-2-oxo-4-(trifluoromethyl)imidazolidin-1-yl)methyl)pyridin-2-yl)acetamide HCl salt (Intermediate 4) as starting material, were prepared:

| Ex. | Acid | Structure | Name | Yield |
|---|---|---|---|---|
| 8 | | | 4-cyclopropyl-N-((S)-1-(4,4- | 116 mg, |
| | | | difluorocyclohexyl)-2-oxo-2- | 74% |
| | | | ((4-(((S)-2-oxo-4- | |
| | | | (trifluoromethyl)imidazolidin- | |
| | | | 1-yl)methyl)pyridin-2- | |
| | | | yl)amino)ethyl)-1,2,5- | |
| | | | oxadiazole-3-carboxamide | |
| 9 | | | N-((S)-1-(4,4- | 12.3 mg, |
| | | | difluorocyclohexyl)-2-oxo-2- | 53% |
| | | | ((4-(((S)-2-oxo-4- | |
| | | | (trifluoromethyl)imidazolidin- | |
| | | | 1-yl)methyl)pyridin-2- | |
| | | | yl)amino)ethyl)-1-methyl- | |
| | | | 1H-pyrazole-5-carboxamide | |

### Example 8

LC/MS: m/z=572.1 [M+H]⁺; rt: 2.13 min (LC/MS-method A). ¹H NMR (400 MHz, DMSO-de) δ ppm: 10.80 (s, 1H), 9.29 (d, J=8.0 Hz, 1H), 8.30 (d, J=5.2 Hz, 1H), 7.99 (s, 1H), 7.65 (d, J=2.0 Hz, 1H), 6.97 (dd, J=5.2 Hz, 1.1 Hz, 1H), 4.74 (dd, J=8.0 Hz, 7.8 Hz, 1H), 4.41 (m, 1H), 4.39 (d, J=16.2 Hz, 1H),), 4.23 (d, J=16.2 Hz, 1H), 3.59 (dd, J=10.0 Hz, 10.0 Hz, 1H), ~3.30 (dd, 1H, signal below water signal), 2.27 (m, 1H), 2.09-1.99 (m, 3H), 1.88-1.68 (m, 4H), 1.49 (m, 1H), 1.37 (m, 1H), 1.13 (m, 2H), 0.98 (m, 2H).

### Example 9

LC/MS: m/z=544.2 [M+H]⁺; rt: 1.71 min (LC/MS-method A). ¹H NMR (400 MHz, DMSO-de) δ ppm: 10.70 (s, 1H), 8.55 (d, J=8.0 Hz, 1H), 8.30 (d, J=5.2 Hz, 1H), 7.99 (s, 1H), 7.64 (d, J=2.0 Hz, 1H), 7.47 (d, J=2.0 Hz, 1H), 7.03 (d, J=2.0 HZ, 1H), 6.95 (dd, J=5.2 Hz, 1.1 Hz, 1H), 4.64 (dd, J=8.6 Hz, 8.0 Hz, 1H), 4.41 (m, 1H), 4.38 (d, J=16.2 Hz, 1H),), 4.22 (d, J=16.2 Hz, 1 H), 4.02 (s, 3H), 3.58 (dd, J=10.0 Hz, 10.0 Hz, 1H), ~3.30 (dd, 1H, signal below water signal), 2.07-1.65 (m, 7H), 1.48 (m, 1H), 1.35 (m, 1H).

### Example 10: Benzyl ((S)-1-(4,4-difluorocyclohexyl)-2-oxo-2-((4-(((S)-2-oxo-4-(trifluoromethyl)imidazolidin-1-yl)methyl)pyridin-2-yl)amino)ethyl)carbamate

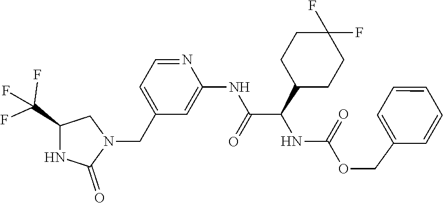 [SMILES: O=C(N[C@@H](C(=O)Nc1cc(CN2C[C@H](C(F)(F)F)NC2=O)ccn1)C1CCC(F)(F)CC1)OCc1ccccc1]

(S)-2-Amino-2-(4,4-difluorocyclohexyl)-N-(4-(((S)-2-oxo-4-(trifluoromethyl)-imidazolidin-1-yl)methyl)pyridin-2-yl)acetamide HCl salt (Intermediate 4; 20 mg) was dissolved in dry DCM (2.5 ml) and with stirring DIPEA (29 μl) was added. After cooling to 0° C., benzyl chloroformate (9 mg, dissolved in 0.5 ml DCM) was added. Then the ice bath was removed, and after 1 h saturated NaHCO₃ solution was added, and the aqueous phase was extracted with DCM (3×). The combined organic phases were dried over sodium sulphate, filtered and concentrated in vacuo. The residue was purified by preparative RP HPLC (Purosphere® STAR-RP18, 25 mm×250 mm, 10 μm; 30 ml/min, from 95% H₂O/5% ACN to 10% H₂O/90% ACN in 45 min). The pure product containing fractions were combined, the ACN was partly removed, and the residue freeze dried to yield 18 mg of the title compound. LC/MS: m/z=570.1 [M+H]⁺; rt: 2.09 min (LC/MS-method A). ¹H NMR (400 MHz, DMSO-d₆) δ ppm: 10.56 (s, 1H), 8.28 (d, J=5.2 Hz, 1H), 7.98 (bs, 1H), 7.65 (d, J=2.0 Hz, 1H), 7.62 (d, J=8.3 Hz, 1H), 7.36-7.30 (m, 5H), 6.95 (dd, J=5.2 Hz, 1.1 Hz, 1H), 5.03 (s, 2H), 4.42 (m, 1H), 4.38 (d, J=16.2 Hz, 1H), 4.29 (dd, J=8.3 Hz, 7.6 Hz, 1H), 4.23 (d, J=16.2 Hz, 1H), 3.59 (dd, J=10.0 Hz, 10.0 Hz, 1H), ~3.30 (dd, 1H, signal below water signal), 2.01 (m, 2H), 1.84-1.60 (m, 5H), 1.47-1.29 (m, 2H).

### Example 11: Tert-butyl 6-(((S)-1-(4,4-difluorocyclohexyl)-2-oxo-2-((4-(((S)-2-oxo-4-(trifluoromethyl)imidazolidin-1-yl)methyl)pyridin-2-yl)amino)ethyl)carbamoyl)-3,4-dihydropyrrolo[1,2-a]pyrazine-2(1H)-carboxylate

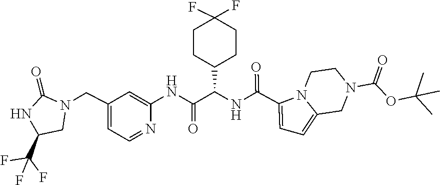 [SMILES: CC(C)(C)OC(=O)N1CCn2c(ccc2C(=O)N[C@H](C(=O)Nc2cc(CN3C[C@@H](C(F)(F)F)NC3=O)ccn2)C2CCC(F)(F)CC2)C1]

2-Tert-butoxycarbonyl-3,4-dihydro-1H-pyrrolo[1,2-a]pyrazine-6-carboxylic acid (14 mg) was dissolved in dry DMF (1.5 ml), and DIPEA (29 μl) and HATU (32 mg) were added with stirring. After 15 min (S)-2-amino-2-(4,4-difluorocyclohexyl)-N-(4-(((S)-2-oxo-4-(trifluoromethyl)imidazolidin-1-yl)methyl)pyridin-2-yl)acetamide HCl salt (Intermediate 4; 20 mg) was added. After standing overnight saturated NaHCO₃ solution was added and the aqueous mixture was extracted with EA (3×). The combined organic phases were dried over sodium sulphate, filtered and concentrated in vacuo. The residue was purified by preparative RP HPLC (Purosphere® STAR-RP18, 25 mm×250 mm, 10 μm; 30 ml/min, from 95% H₂O/5% ACN to 10% H₂O/90% ACN in 45 min). The pure product containing fractions were combined, the ACN was partly removed, and the residue freeze dried to yield 16 mg of the title compound. LC/MS: m/z=684.2 [M+H]⁺; rt: 2.31 min (LC/MS-method A). ¹H NMR (400 MHz, DMSO-d₆) δ ppm: 10.56 (s, 1H), 8.28 (d, J=5.2 Hz, 1H), 7.99 (bs, 1H), 7.97 (d, J=8.5 Hz, 1H), 7.64 (d, J=2.0 Hz, 1H), 7.00 (d, J=3.9 Hz, 1H), 6.94 (dd, J=5.2 Hz, 1.1 Hz, 1H), 5.96 (d, J=3.9 Hz, 1H), 4.56 (dd, overlapped, 1H), 4.53 (s, 2H), 4.41 (m, 1H), 4.37 (d, J=16.2 Hz, 1H), 4.30 (m, 2H), 4.22 (d, J=16.2 Hz, 1H), 3.63 (m, 2H), 3.58 (dd, J=10.0 Hz, 10.0 Hz, 1H), ~3.30 (dd, 1H, signal below water signal), 2.07-1.64 (m, 7H), 1.49-1.27 (m, 2H), 1.42 (s, 9H).

Analogously to Example 3, using (S)-2-amino-2-(4,4-difluorocyclohexyl)-N-(4-(((S)-2-oxo-4-(trifluoromethyl)imidazolidin-1-yl)methyl)pyridin-2-yl)acetamide HCl salt (Intermediate 4) as starting material, were prepared:

| Ex. | Acid | Structure | Name | Yield |
|---|---|---|---|---|
| 12 | | | N-((S)-1-(4,4- | 17 mg, |
| | | | difluorocyclohexyl)-2-oxo- | 69% |
| | | | 2-((4-(((S)-2-oxo-4- | |
| | | | (trifluoromethyl)imidazolidin- | |
| | | | 1-yl)methyl)pyridin-2- | |
| | | | yl)amino)ethyl)-1- | |
| | | | isopropyl-1H-pyrazole-5- | |
| | | | carboxamide | |
| 13 | | | N-((S)-1-(4,4- | 16 mg, |
| | | | difluorocyclohexyl)-2-oxo- | 69% |
| | | | 2-((4-(((S)-2-oxo-4- | |
| | | | (trifluoromethyl)imidazolidin- | |
| | | | 1-yl)methyl)pyridin-2- | |
| | | | yl)amino)ethyl)-3- | |
| | | | ethylisoxazole-4- | |
| | | | carboxamide | |
| 14 | | | 1-cyclopropyl-N-((S)-1- | 16 mg, |
| | | | (4,4-difluorocyclohexyl)-2- | 66% |
| | | | oxo-2-((4-(((S)-2-oxo-4- | |
| | | | (trifluoromethyl)imidazolidin- | |
| | | | 1-yl)methyl)pyridin-2- | |
| | | | yl)amino)ethyl)-1H- | |
| | | | pyrazole-5-carboxamide | |

### Example 12

LC/MS: m/z=572.2 [M+H]⁺; rt: 1.94 min (LC/MS-method A). ¹H NMR (400 MHz, DMSO-d6) δ ppm: 10.69 (s, 1H), 8.55 (d, J=7.9 Hz, 1H), 8.30 (d, J=5.2 Hz, 1H), 7.99 (bs, 1H), 7.64 (d, J=2.0 Hz, 1H), 7.50 (d, J=1.9 Hz, 1H), 6.96 (dd, J=5.2 Hz, 1.1 Hz, 1H), 6.93 (d, J=1.9 Hz, 1H), 5.36 (m, 1H), 4.62 (dd, J=8.6 Hz, 7.9 Hz, 1H), 4.41 (m, 1H), 4.38 (d, J=16.2 Hz, 1H), 4.22 (d, J=16.2 Hz, 1H), 3.58 (dd, J=10.0 Hz, 10.0 Hz, 1H), ~3.30 (dd, 1H, signal below water signal), 2.08-1.66 (m, 7H), 1.52-1.32 (m, 2H), 1.36 (d, J=6.6 Hz, 3H), 1.34 (d, J=6.6 Hz, 3H).

### Example 13

LC/MS: m/z=559.2 [M+H]⁺; rt: 1.89 min (LC/MS-method A). ¹H NMR (400 MHz, DMSO-d6) δ ppm: 10.71 (s, 1H), 9.40 (s, 1H), 8.52 (d, J=8.0 Hz, 1H), 8.29 (d, J=5.2 Hz, 1H), 7.99 (bs, 1H), 7.64 (d, J=2.0 Hz, 1H), 6.96 (dd, J=5.2 Hz, 1.1 Hz, 1H), 4.67 (dd, J=8.2 Hz, 8.0 Hz, 1H), 4.41 (m, 1H), 4.38 (d, J=16.2 Hz, 1H), 4.22 (d, J=16.2 Hz, 1H), 3.58 (dd, J=10.0 Hz, 10.0 Hz, 1H), ~3.30 (dd, 1H, signal below water signal), 2.83 (q, J=7.5 Hz, 2H), 2.07-1.66 (m, 7H), 1.52-1.32 (m, 2H), 1.16 (t, J=7.5 Hz, 3H).

### Example 14

LC/MS: m/z=570.2 [M+H]⁺; rt: 1.84 min (LC/MS-method A). ¹H NMR (400 MHz, DMSO-de) δ ppm: 10.70 (s, 1H), 8.57 (d, J=8.0 Hz, 1H), 8.30 (d, J=5.2 Hz, 1H), 8.00 (bs, 1H), 7.64 (d, J=2.0 Hz, 1H), 7.42 (d, J=2.0 Hz, 1H), 6.96 (dd, J=5.2 Hz, 1.1 Hz, 1H), 6.94 (d, J=2.0 Hz, 1H), 4.66 (dd, J=8.4 Hz, 8.0 Hz, 1H), 4.41 (m, 1H), 4.39 (m, 1H), 4.38 (d, J=16.2 Hz, 1H), 4.22 (d, J=16.2 Hz, 1H), 3.58 (dd, J=10.0 Hz, 10.0 Hz, 1H), ~3.30 (dd, 1H, signal below water signal), 2.09-1.67 (m, 7H), 1.49 (m, 1H), 1.36 (m, 1H), 1.05 (m, 2H), 0.92 (m, 2H).

Analogously to Example 2, using (S)-2-amino-3,3-dicyclopropyl-N-(4-(((S)-2-oxo-4-(trifluoromethyl)imidazolidin-1-yl)methyl)pyridin-2-yl)propanamide HCl salt (Intermediate 6) as starting material, were prepared:

| Ex. | Acid | Structure | Name | Yield |
|---|---|---|---|---|
| 15 | | | N-((S)-1,1-dicyclopropyl- | 16 mg, |
| | | | 3-oxo-3-((4-(((S)-2-oxo-4- | 74% |
| | | | (trifluoromethyl)imidazolidin- | |
| | | | 1-yl)methyl)pyridin-2- | |
| | | | yl)amino)propan-2-yl)-1- | |
| | | | methyl-1H-pyrazole-5- | |
| | | | carboxamide | |
| 16 | | | 4-cyclopropyl-N-((S)-1,1- | 17 mg, |
| | | | dicyclopropyl-3-oxo-3-((4- | 74% |
| | | | (((S)-2-oxo-4- | |
| | | | (trifluoromethyl)imidazolidin- | |
| | | | 1-yl)methyl)pyridin-2- | |
| | | | yl)amino)propan-2-yl)- | |
| | | | 1,2,5-oxadiazole-3- | |
| | | | carboxamide | |
| 17 | | | N-((S)-1,1-dicyclopropyl- | 16 mg, |
| | | | 3-oxo-3-((4-(((S)-2-oxo-4- | 70% |
| | | | (trifluoromethyl)imidazolidin- | |
| | | | 1-yl)methyl)pyridin-2- | |
| | | | yl)amino)propan-2-yl)-1- | |
| | | | isopropyl-1H-pyrazole-5- | |
| | | | carboxamide | |
| 18 | | | N-((S)-1,1-dicyclopropyl- | 15 mg, |
| | | | 3-oxo-3-((4-(((S)-2-oxo-4- | 71% |
| | | | (trifluoromethyl)imidazolidin- | |
| | | | 1-yl)methyl)pyridin-2- | |
| | | | yl)amino)propan-2-yl)-4- | |
| | | | methyl-1,2,5-oxadiazole- | |
| | | | 3-carboxamide | |

### Example 15

LC/MS: m/z=520.2 [M+H]⁺; rt: 1.80 min (LC/MS-method A). ¹H NMR (400 MHz, DMSO-de) δ ppm: 10.66 (s, 1H), 8.39 (d, J=8.7 Hz, 1H), 8.28 (d, J=5.2 Hz, 1H), 7.98 (bs, 1H), 7.64 (d, J=2.0 Hz, 1H), 7.47 (d, J=2.0 Hz, 1H), 7.03 (d, J=2.0 Hz, 1H), 6.94 (dd, J=5.2 Hz, 1.1 Hz, 1H), 4.91 (dd, J=8.7 Hz, 7.4 Hz, 1H), 4.43 (d, J=16.2 Hz, 1H), 4.41 (m, 1H), 4.19 (d, J=16.2 Hz, 1H), 4.03 (s, 3H), 3.58 (dd, J=10.0 Hz, 10.0 Hz, 1H), 3.29 (dd, 1H, signal below water signal), 0.94 (m, 1H), 0.84 (m, 1H), 0.76 (m, 1H), 0.45 (m, 1H), 0.36 (m, 1H), 0.30-0.12 (m, 6H).

### Example 16

LC/MS: m/z=548.2 [M+H]⁺; rt: 2.26 min (LC/MS-method A). ¹H NMR (400 MHz, DMSO-de) δ ppm: 10.75 (s, 1H), 9.14 (d, J=8.7 Hz, 1H), 8.29 (d, J=5.2 Hz, 1H), 7.98 (bs, 1H), 7.65 (d, J=2.0 Hz, 1H), 6.95 (dd, J=5.2 Hz, 1.1 Hz, 1H), 4.99 (dd, J=8.7 Hz, 6.5 Hz, 1H), 4.44 (d, J=16.2 Hz, 1H), 4.41 (m, 1H), 4.20 (d, J=16.2 Hz, 1H), 3.59 (dd, J=10.0 Hz, 10.0 Hz, 1H), 3.30 (dd, 1H, signal below water signal), 2.28 (m, 1H), 1.13 (m, 2H), 0.99 (m, 2H), 0.92 (m, 1H), 0.83 (m, 1H), 0.76 (m, 1H), 0.48 (m, 1H), 0.38 (m, 1H), 0.30 (m, 1H), 0.23-0.15 (m, 5H).

### Example 17

LC/MS: m/z=548.1 [M+H]⁺; rt: 2.03 min (LC/MS-method A). ¹H NMR (400 MHz, DMSO-de) δ ppm: 10.64 (s, 1H), 8.39 (d, J=8.6 Hz, 1H), 8.28 (d, J=5.2 Hz, 1H), 7.98 (bs, 1H), 7.64 (d, J=2.0 Hz, 1H), 7.50 (d, J=1.9 Hz, 1H), 6.94 (dd, J=5.2 Hz, 1.1 Hz, 1H), 6.91 (d, J=1.9 Hz, 1H), 5.38 (m, 1H), 4.90 (dd, J=8.6 Hz, 7.5 Hz, 1H), 4.44 (d, J=16.2 Hz, 1H), 4.41 (m, 1H), 4.19 (d, J=16.2 Hz, 1H), 3.58 (dd, J=10.0 Hz, 10.0 Hz, 1H), 3.30 (dd, 1H, signal below water signal), 1.37 (d, J=6.6 Hz, 3H), 1.34 (d, J=6.6 Hz, 3H), 0.94 (m, 1H), 0.84 (m, 1H), 0.74 (m, 1H), 0.45 (m, 1H), 0.36 (m, 1H), 0.31-0.13 (m, 6H).

### Example 18

LC/MS: m/z=522.1 [M+H]⁺; rt: 2.08 min (LC/MS-method A). ¹H NMR (400 MHz, DMSO-de) δ ppm: 10.75 (s, 1H), 9.04 (d, J=8.8 Hz, 1H), 8.28 (d, J=5.1 Hz, 1H), 7.98 (bs, 1H), 7.65 (d, J=2.0 Hz, 1H), 6.95 (dd, J=5.1 Hz, 1.1 Hz, 1H), 4.97 (dd, J=8.8 Hz, 6.5 Hz, 1H), 4.43 (d, J=16.2 Hz, 1H), 4.41 (m, 1H), 4.20 (d, J=16.2 Hz, 1H), 3.59 (dd, J=10.0 Hz, 10.0 Hz, 1H), 3.30 (dd, 1H, signal below water signal), 2.48 (s, 3H), 0.91 (m, 1H), 0.85-0.73 (m, 2H), 0.47 (m, 1H), 0.38 (m, 1H), 0.29 (m, 1H), 0.23-0.17 (m, 5H).

### Example 19: Tert-butyl ((1-(4-(((S)-1-(4,4-difluorocyclohexyl)-2-oxo-2-((4-(((S)-2-oxo-4-(trifluoromethyl)imidazolidin-1-yl)methyl)pyridin-2-yl)amino)ethyl)-carbamoyl)isoxazol-3-yl)cyclopropyl)methyl)carbamate

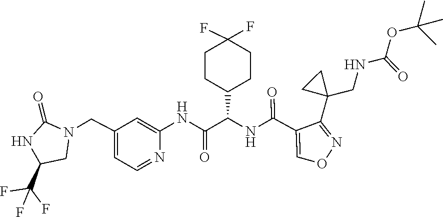 [SMILES: CC(C)(C)OC(=O)NCC1(c2nocc2C(=O)N[C@H](C(=O)Nc2cc(CN3C[C@@H](C(F)(F)F)NC3=O)ccn2)C2CCC(F)(F)CC2)CC1]

To a mixture of (S)-2-amino-2-(4,4-difluorocyclohexyl)-N-(4-(((S)-2-oxo-4-(trifluoromethyl)imidazolidin-1-yl)methyl)pyridin-2-yl)acetamide HCl salt (Intermediate 4; 30 mg), 3-[1-[(tert-butoxycarbonylamino)methyl]cyclopropyl]isoxazole-4-carboxylic acid (22 mg) and DCM (3.5 ml), DIPEA (44 μl) and T3P (133 μl) were added. After stirring for 2 h and standing overnight, the mixture was concentrated in vacuo. The residue was purified by preparative RP HPLC (Purosphere® STAR-RP18, 25 mm×250 mm, 10 μm; 30 ml/min, from 95% H₂O/5% ACN to 10% H₂O/90% ACN in 45 min). The pure product containing fractions were combined, the ACN was partly removed, and the residue freeze dried to yield 28 mg of the title compound. LC/MS: m/z=700.3 [M+H]⁺; rt: 2.13 min (LC/MS-method A). ¹H NMR (400 MHz, DMSO-d₆) δ ppm: 10.72 (s, 1H), 9.31 (s, 1H), 8.36 (d, J=8.0 Hz, 1H), 8.30 (d, J=5.1 Hz, 1H), 7.99 (bs, 1H), 7.65 (d, J=2.0 Hz, 1H), 6.96 (dd, J=5.1 Hz, 1.1 Hz, 1H), 6.80 (t, J=6.0 Hz, 1H), 4.74 (dd, J=8.0 Hz, 7.8 Hz, 1H), 4.40 (m, 1H), 4.39 (d, J=16.2 Hz, 1H), 4.22 (d, J=16.2 Hz, 1H), 3.58 (dd, J=10.0 Hz, 10.0 Hz, 1H), ~3.33 (dd, J=14.0 Hz, 6.0 Hz, 1H), ~3.30 (dd, 1H, signal below water signal), 3.21 (dd, J=14.0 Hz, 6.0 Hz, 1H), 2.07-1.68 (m, 7H), 1.52-1.33 (m, 2H), 1.28/1.16 (s, 9H), 0.88-0.81 (m, 4H).

Analogously to Example 3, using (S)-2-amino-2-(4,4-difluorocyclohexyl)-N-(4-(((S)-2-oxo-4-(trifluoromethyl)imidazolidin-1-yl)methyl)pyridin-2-yl)acetamide HCl salt (Intermediate 4) as starting material, were prepared:

| Ex. | Acid | Structure | Name | Yield |
|---|---|---|---|---|
| 20 | | | 3-cyclopropyl-N-((S)-1- | 17 mg, |
| | | | (4,4-difluorocyclohexyl)-2- | 69% |
| | | | oxo-2-((4-(((S)-2-oxo-4- | |
| | | | (trifluoromethyl)- | |
| | | | imidazolidin-1-yl)methyl)- | |
| | | | pyridin-2-yl)amino)ethyl)- | |
| | | | isoxazole-4-carboxamide | |
| 21 | | | N-((S)-1-(4,4- | 16 mg, |
| | | | difluorocyclohexyl)-2-oxo-2- | 64% |
| | | | ((4-(((S)-2-oxo-4-(trifluoro- | |
| | | | methyl)imidazolidin-1- | |
| | | | yl)methyl)pyridin-2- | |
| | | | yl)amino)ethyl)-3-isopropyl- | |
| | | | isoxazole-4-carboxamide | |

### Example 20

LC/MS: m/z=571.1 [M+H]⁺; rt: 1.93 min (LC/MS-method A). ¹H NMR (400 MHz, DMSO-de) δ ppm: 10.72 (s, 1H), 9.37 (s, 1H), 8.50 (d, J=8.0 Hz, 1H), 8.30 (d, J=5.1 Hz, 1H), 7.99 (bs, 1H), 7.64 (d, J=2.0 Hz, 1H), 6.96 (dd, J=5.1 Hz, 1.1 Hz, 1H), 4.68 (dd, J=8.0 Hz, 8.0 Hz, 1H), 4.41 (m, 1H), 4.39 (d, J=16.2 Hz, 1H), 4.22 (d, J=16.2 Hz, 1H), 3.58 (dd, J=10.0 Hz, 10.0 Hz, 1H), ~3.30 (dd, 1H, signal below water signal), 2.43 (m, 1H), 2.08-1.66 (m, 7H), 1.48 (m, 1H), 1.37 (m, 1H), 0.99 (m, 2H), 0.89 (m, 2H).

### Example 21

LC/MS: m/z=573.1 [M+H]⁺; rt: 2.01 min (LC/MS-method A). ¹H NMR (400 MHz, DMSO-de) δ ppm: 10.70 (s, 1H), 9.35 (s, 1H), 8.54 (d, J=8.0 Hz, 1H), 8.29 (d, J=5.1 Hz, 1H), 7.98 (bs, 1H), 7.64 (d, J=2.0 Hz, 1H), 6.96 (dd, J=5.1 Hz, 1.1 Hz, 1H), 4.67 (dd, J=8.1 Hz, 8.0 Hz, 1H), 4.41 (m, 1H), 4.39 (d, J=16.2 Hz, 1H), 4.21 (d, J=16.2 Hz, 1H), 3.58 (dd, J=10.0 Hz, 10.0 Hz, 1H), 3.41 (m, 1H), ~3.30 (dd, 1H, signal below water signal), 2.08-1.66 (m, 7H), 1.47 (m, 1H), 1.36 (m, 1H), 1.23 (d, J=7.0 Hz, 3H), 1.21 (d, J=7.0 Hz, 3H).

### Example 22: 3-(1-(Aminomethyl)cyclopropyl)-N—((S)-1-(4,4-difluorocyclohexyl)-2-oxo-2-((4-(((S)-2-oxo-4-(trifluoromethyl)imidazolidin-1-yl)methyl)pyridin-2-yl)amino)ethyl)isoxazole-4-carboxamide HCl salt

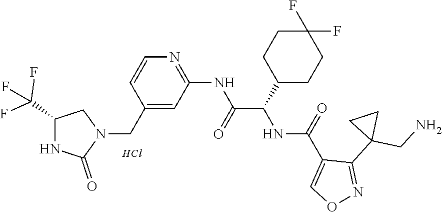 [SMILES: NCC1(c2nocc2C(=O)N[C@H](C(=O)Nc2cc(CN3C[C@@H](C(F)(F)F)NC3=O)ccn2)C2CCC(F)(F)CC2)CC1 ; Cl]

Following the procedure of Intermediate 4, tert-butyl N-[[1-[4-[[(1S)-1-(4,4-difluorocyclohexyl)-2-oxo-2-[[4-[[(4S)-2-oxo-4-(trifluoromethyl)imidazolidin-1-yl]methyl]-2-pyridyl]amino]ethyl]carbamoyl]isoxazol-3-yl]cyclopropyl]methyl]carbamate (Example 19; 22 mg) gave 20 mg of the title compound. LC/MS: m/z=600.2 [M+H]⁺; rt: 1.27 min (LC/MS-method A). ¹H NMR (400 MHz, DMSO-de) δ ppm: 10.79 (s, 1H), 9.51 (s, 1H), 8.67 (d, J=8.0 Hz, 1H), 8.30 (d, J=5.1 Hz, 1H), 7.98 (bs, 1H), 7.87 (bt, 3H), 7.66 (d, J=2.0 Hz, 1H), 6.98 (dd, J=5.1 Hz, 1.1 Hz, 1H), 4.69 (dd, J=8.0 Hz, 8.0 Hz, 1H), 4.42 (m, 1H), 4.41 (d, J=16.2 Hz, 1H), 4.21 (d, J=16.2 Hz, 1H), 3.60 (dd, J=10 Hz, 10 Hz, 1H), 3.32 (dd, J=10.0 Hz, 4.1 Hz, 1H), 3.17 (m, 2H), 2.08-1.67 (m, 7H), 1.50 (m, 1H), 1.37 (m, 1H), 1.08-1.04 (m, 4H).

### Example 23: Tert-butyl ((S)-2-((4-((5-methyl-6-oxo-5,7-diazaspiro[3.4]octan-7-yl)methyl)pyridin-2-yl)amino)-1-((1r,4S)-4-methylcyclohexyl)-2-oxoethyl)carbamate

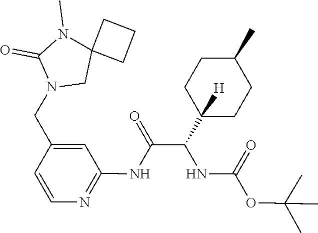

Tert-butyl ((S)-2-((4-((((1-(methylamino)cyclobutyl)methyl)amino)methyl)pyridin-2-yl)amino)-1-((1r,4S)-4-methylcyclohexyl)-2-oxoethyl)carbamate (Intermediate 7, 22 mg) was dissolved in dry THE (3 ml) at RT under Ar. DIPEA (32 μl) was added. After heating for 5 min at 65° C., CDI (22 mg) was added. After stirring for 3 d at 65° C., the mixture was cooled, DCM and saturated NaHCO₃ solution were added, and the aqueous mixture was extracted with DCM (2×). The combined organic phases were dried over sodium sulphate, filtered, and concentrated in vacuo. The residue was purified by preparative RP HPLC (Purosphere® STAR-RP18, 25 mm×250 mm, 10 μm; 30 ml/min, from 95% H₂O/5% ACN to 10% H₂O/90% ACN in 45 min). The pure product containing fractions were combined, the ACN was partly removed, and the residue freeze dried to yield 11 mg of the title compound. LC/MS: m/z=500.3 [M+H]⁺; rt: 2.51 min (LC/MS-method A). ¹H NMR (400 MHz, DMSO-d₆) δ ppm: 10.32 (s, 1H), 8.25 (d, J=5.1 Hz, 1H), 7.97 (bs, 1H), 6.94 (dd, J=5.1 Hz, 1.1 Hz, 1H), ~6.92 (bd, overlapped, 1H), 4.28 (s, 2H), 4.04 (bdd, 1H), ~3.30 (signal below water signal), 2.74 (s, 3H), ~2.45 (m, 2H), 1.82 (m, 2H), 1.71-1.49 (m, 7H), 1.37/1.29 (s, 9H), 1.28-1.11 (m, 2H), 1.03 (m, 1H), 0.86-0.77 (m, 2H), 0.83 (d, J=6.5 Hz, 3H).

### Example 24: Tert-butyl ((S)-2-((4-((4,4-dimethyl-2-oxoimidazolidin-1-yl)methyl)pyridin-2-yl)amino)-1-((1r,4S)-4-methylcyclohexyl)-2-oxoethyl)carbamate

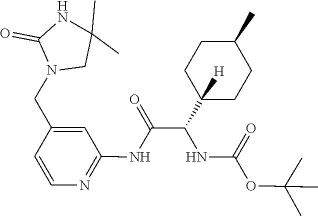

Tert-butyl ((S)-2-((4-(((2-amino-2-methylpropyl)amino)methyl)pyridin-2-yl)amino)-1-((1r,4S)-4-methylcyclohexyl)-2-oxoethyl)carbamate (Intermediate 8, 20 mg) was dissolved in dry THE (2 ml) at RT under Ar. DIPEA (31 μl) was added. After heating for 5 min at 65° C., CDI (22 mg) was added. After stirring for 4 h at 65° C. and standing overnight, DCM was added, and the mixture was poured into saturated NaHCO₃ solution. The aqueous mixture was extracted with DCM (2×). The combined organic phases were dried over sodium sulphate, filtered, and concentrated in vacuo. The residue was purified by preparative RP HPLC (Agilent Prep C18, 30 mm×100 mm, 5 μm; 40 ml/min, from 97% H₂O/3% ACN to 10% H₂O/90% ACN in 12.5 min). The pure product containing fractions were combined, the ACN was partly removed, and the residue freeze dried to yield 17 mg of the title compound. LC/MS: m/z=474.3 [M+H]⁺; rt: 2.27 min (LC/MS-method A). ¹H NMR (400 MHz, DMSO-d₆) δ ppm: 10.31 (s, 1H), 8.25 (d, J=5.1 Hz, 1H), 7.99 (bs, 1H), 6.95 (bd, J=5.1 Hz, 1H), 6.92 (bd, J=8.4 Hz, 1H), 6.67 (s, 1H), 4.25 (s, 2H), 4.04 (bdd, 1H), 3.00 (s, 2H), 1.71-1.49 (m, 5H), 1.37/1.29 (s, 9H), 1.28-1.11 (m, 2H), 1.19 (s, 6H), 1.03 (m, 1H), 0.86-0.77 (m, 2H), 0.83 (d, J=6.5 Hz, 3H).

### Example 25: 4-Cyclopropyl-N—((S)-2-((4-((4,4-dimethyl-2-oxoimidazolidin-1-yl)methyl)pyridin-2-yl)amino)-1-((1r,4S)-4-methylcyclohexyl)-2-oxoethyl)-1,2,5-oxadiazole-3-carboxamide

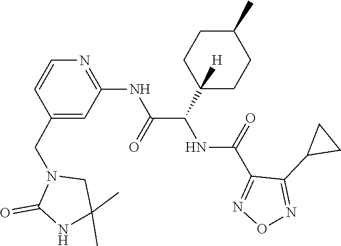

To a mixture of (S)-2-amino-N-(4-((4,4-dimethyl-2-oxoimidazolidin-1-yl)methyl)pyridin-2-yl)-2-((1r,4S)-4-methylcyclohexyl)acetamide HCl salt (Intermediate 9, 12 mg), 4-cyclopropyl-1,2,5-oxadiazole-3-carboxylic acid (5 mg) and DCM (1 ml), DIPEA (25 μl) and T3P (52 μl) were added. After stirring overnight, the mixture was concentrated in vacuo. The residue was purified by preparative RP HPLC (Agilent Prep C18, 30 mm×100 mm, 5 μm; 40 ml/min, from 97% H₂O/3% ACN to 10% H₂O/90% ACN in 12.5 min). The pure product containing fractions were combined, the ACN was partly removed, and the residue freeze dried to yield 6 mg of the title compound. LC/MS: m/z=510.2 [M+H]⁺; rt: 2.38 min (LC/MS-method A). ¹H NMR (400 MHz, DMSO-de) δ ppm: 10.70 (s, 1H), 9.16 (d, J=8.1 Hz, 1H), 8.28 (d, J=5.1 Hz, 1H), 8.00 (bs, 1H), 6.97 (dd, J=5.1 Hz, 1.1 Hz, 1H), 6.67 (s, 1H), 4.62 (dd, J=8.1 Hz, 7.8 Hz, 1H), 4.26 (s, 2H), 3.01 (s, 2H), 2.26 (m, 1H), 1.83-1.58 (m, 5H), 1.29-1.19 (m, 2H), 1.19 (s, 6H), 1.12 (m, 2H), 1.08 (m, 1H), 0.97 (m, 2H), 0.91-0.82 (m, 2H), 0.85 (d, J=6.5 Hz, 3H).

### Example 26: Tert-butyl ((S)-1-((1r,4S)-4-methylcyclohexyl)-2-oxo-2-((4-((((2,2,2-trifluoroethyl)carbamoyl)oxy)methyl)pyridin-2-yl)amino)ethyl)carbamate

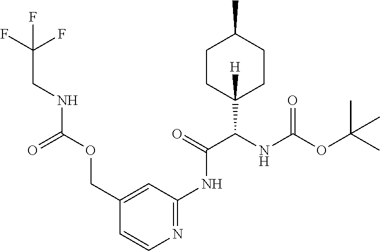

(2-Aminopyridin-4-yl)methyl (2,2,2-trifluoroethyl)carbamate (Intermediate 10, 19 mg) and (2S)-2-(tert-butoxycarbonylamino)-2-(trans-4-methylcyclohexyl)acetic acid (20 mg) were dissolved in DCM. EDC (17 mg) and DMAP (11 mg) were added with stirring. After stirring for 3 d, the mixture was poured onto saturated NaHCO₃ solution, and the aqueous phase was extracted with DCM (2×). The combined organic phases were dried (Chem Elut cartridge) and concentrated in vacuo. The residue was purified by silica gel chromatography (Silica gel 4 g, hep/EA: 100:0 for 5 min, 100:0 to 50:50 in 30 min, 50:50 for 10 min). The pure compound containing fractions were combined, and concentrated in vacuo. The residue was dissolved in ACN/water and freeze dried to yield 3 mg of the title compound. LC/MS: m/z=503.2 [M+H]⁺; rt: 2.52 min (LC/MS-method A). ¹H NMR (400 MHz, DMSO-d₆) δ ppm: 10.36 (s, 1H), 8.29 (d, J=5.1 Hz, 1H), 8.22 (t, J=6.2 Hz, 1H), 8.01 (bs, 1H), 7.04 (bd, J=5.1 Hz, 1H), 6.92 (bd, J=8.3 Hz, 1H), 5.12 (s, 2H), 4.05 (bdd, 1H), 3.82 (m, 2H), 1.72-1.48 (m, 5H), 1.37 (s, 9H), 1.28-1.11 (m, 2H), 1.03 (m, 1H), 0.86-0.77 (m, 2H), 0.83 (d, J=6.5 Hz, 3H).

### Example 27: Tert-butyl (S)-(1-(4,4-difluorocyclohexyl)-2-((4-((4,4-dimethyl-2,5-dioxoimidazolidin-1-yl)methyl)pyridin-2-yl)amino)-2-oxoethyl)carbamate

Tert-butyl (S)-(2-((4-(bromomethyl)pyridin-2-yl)amino)-1-(4,4-difluorocyclohexyl)-2-oxoethyl)carbamate (Intermediate 11, 9 mg) was dissolved in dry DMF (2 ml), and potassium carbonate (3 mg) and 5,5-dimethylimidazolidine-2,4-dione (2.5 mg) were added with stirring. After heating at 90° C. for 1.5 h, the heating was stopped, and the mixture stood overnight. The mixture was directly purified by preparative RP HPLC (Purosphere® STAR-RP18, 25 mm×250 mm, 10 μm; 30 ml/min, from 95% H2O/5% ACN to 10% H2O/90% ACN in 45 min). The pure product containing fractions were combined, the ACN was partly removed, and the residue freeze dried to yield 5 mg of the title compound. LC/MS: m/z=510.2 [M+H]⁺; rt: 1.97 min (LC/MS-method A). ¹H NMR (400 MHz, DMSO-de) δ ppm: 10.47 (s, 1H), 8.46 (s, 1H), 8.26 (d, J=5.1 Hz, 1H), 7.91 (bs, 1H), 7.07 (bd, J=8.3 Hz, 1H), 6.97 (dd, J=5.1 Hz, 1.1 Hz, 1H), 4.55 (s, 2H), 4.17 (bdd, 1H), 2.00 (m, 2H), 1.81-1.57 (m, 5H), 1.42-1.29 (m, 2H), 1.37 (s, 9H), 1.35 (s, 6H).

### Example 28: Tert-butyl (S)-(1-(4,4-difluorocyclohexyl)-2-((4-((2,5-dioxoimidazolidin-1-yl)methyl)pyridin-2-yl)amino)-2-oxoethyl)carbamate

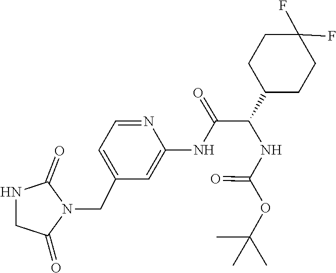 [SMILES: CC(C)(C)OC(=O)N[C@H](C(=O)Nc1cc(CN2C(=O)CNC2=O)ccn1)C1CCC(F)(F)CC1]

Following the procedure in Example 27, using imidazolidine-2,4-dione (2 mg), 4 mg (45%) of the title compound was obtained. LC/MS: m/z=482.2 [M+H]⁺; rt: 1.79 min (LC/MS-method A). ¹H NMR (400 MHz, DMSO-de) 6 ppm: 10.48 (s, 1H), 8.26 (d, J=5.1 Hz, 1H), 8.20 (bs, 1H), 8.00 (bs, 1H), 7.07 (bd, J=8.2 Hz, 1H), 6.98 (dd, J=5.1 Hz, 1.1 Hz, 1H), 4.54 (s, 2H), 4.19 (bdd, 1H), 4.01 (s, 2H), 2.01 (m, 2H), 1.83-1.58 (m, 5H), 1.43-1.29 (m, 2H), 1.38 (s, 9H).

### Examples 29 and 30: Tert-butyl ((S)-2-((4-(((R)-5-methyl-2-oxoimidazolidin-1-yl)methyl)pyridin-2-yl)amino)-1-((1r,4S)-4-methylcyclohexyl)-2-oxoethyl)carbamate and tert-butyl ((S)-2-((4-(((R)-4-methyl-2-oxoimidazolidin-1-yl)methyl)pyridin-2-yl)amino)-1-(r,4S)-4-methylcyclohexyl)-2-oxoethyl)carbamate

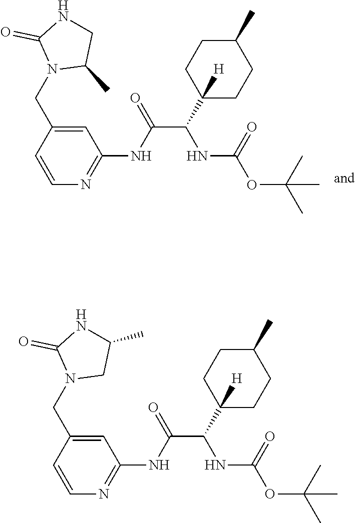

Following the procedure of Example 24 using a mixture of tert-butyl ((S)-2-((4-((((R)-2-aminopropyl)amino)methyl)pyridin-2-yl)amino)-1-((1r,4S)-4-methylcyclohexyl)-2-oxoethyl)carbamate and tert-butyl ((S)-2-((4-((((R)-1-aminopropan-2-yl)amino)-methyl)pyridin-2-yl)amino)-1-((1r,4S)-4-methylcyclohexyl)-2-oxoethyl)carbamate (Intermediate 12, 15 mg), 14 mg of the title compounds were obtained as a mixture. After chiral HPLC separation (Chiralpak IE/164, 250 mm×4.6 mm; nHep:EtOH:MeOH 1:1:1, 1 ml/min; 30° C.), the residues were dissolved in ACN/water and freeze dried to yield 3 mg of tert-butyl N-[(1S)-1-(4-methylcyclohexyl)-2-[[4-[[(5R)-5-methyl-2-oxo-imidazolidin-1-yl]methyl]-2-pyridyl]amino]-2-oxo-ethyl]carbamate (Product 1) and 10 mg of tert-butyl N—[(1S)-1-(4-methylcyclohexyl)-2-[[4-[[(4R)-4-methyl-2-oxo-imidazolidin-1-yl]methyl]-2-pyridyl]amino]-2-oxo-ethyl]carbamate (Product 2).

Product 1: Chiral HPLC (conditions above): rt: 8.26 min. LC/MS: m/z=460.2 [M+H]⁺; rt: 2.15 min (LC/MS-method A). ¹H NMR (400 MHz, DMSO-d₆) δ ppm: 10.30 (s, 1H), 8.24 (d, J=5.1 Hz, 1H), 8.01 (bs, 1H), 6.97 (dd, J=5.1 Hz, 1.1 Hz, 1H), 6.91 (bd, J=8.7 Hz, 1H), 6.48 (s, 1H), 4.34 (d, J=16.4 Hz, 1H), 4.17 (d, J=16.4 Hz, 1H), 4.06 (bdd, 1H), 3.54 (m, 1H), 3.40 (dd, J=8.5 Hz, 8.5 Hz, 1H), 2.84 (ddd, J=8.5 Hz, 8.5 Hz, 1.0 Hz, 1H), 1.71-1.49 (m, 5H), 1.38/1.29 (s, 9H), 1.27-1.12 (m, 2H), 1.08 (d, J=6.0 Hz, 3H), 1.03 (m, 1H), 0.87-0.77 (m, 2H), 0.83 (d, J=6.5 Hz, 3H).

Product 2: Chiral HPLC (conditions above): rt: 10.96 min. LC/MS: m/z=460.3 [M+H]⁺; rt: 2.18 min (LC/MS-method A). ¹H NMR (400 MHz, DMSO-de) δ ppm: 10.32 (s, 1H), 8.25 (d, J=5.1 Hz, 1H), 7.98 (bs, 1H), 6.95 (dd, J=5.1 Hz, 1.1 Hz, 1H), 6.92 (bd, J=8.4 Hz, 1H), 6.65 (s, 1H), 4.24 (s, 2H), 4.05 (bdd, 1H), 3.66 (m, 1H), 3.38 (dd, J=8.5 Hz, 8.5 Hz, 1H), 2.80 (dd, J=8.5 Hz, 6.2 Hz, 1H), 1.71-1.49 (m, 5H), 1.38/1.29 (s, 9H), 1.27-1.12 (m, 2H), 1.11 (d, J=6.1 Hz, 3H), 1.03 (m, 1H), 0.87-0.77 (m, 2H), 0.83 (d, J=6.5 Hz, 3H).

### Example 31: (S)—N-(1,1-dicyclopropyl-3-oxo-3-((4-(2-oxo-2-((3,3,3-trifluoro-propyl)amino)ethyl)pyridin-2-yl)amino)propan-2-yl)-1-methyl-1H-pyrazole-5-carboxamide

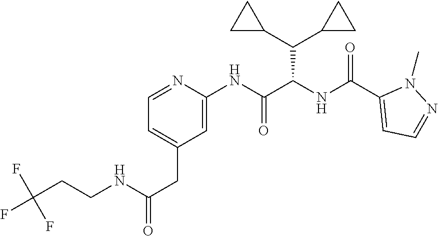 [SMILES: Cn1nccc1C(=O)N[C@H](C(=O)Nc1cc(CC(=O)NCCC(F)(F)F)ccn1)C(C1CC1)C1CC1]

280 mg tert-butyl (S)-(1,1-dicyclopropyl-3-oxo-3-((4-(2-oxo-2-((3,3,3-trifluoro-propyl)amino)ethyl)pyridin-2-yl)amino)propan-2-yl)carbamate (Intermediate 30) was dissolved in 50 ml dry DCM, 580 mg PTSA was added, and the mixture was stirred for 16 h at ambient temperature before evaporation of the solvent. The residue was dissolved in 5.6 ml 0.5 M aqueous NaOH solution and extracted 6 times with 10 ml DCM. The combined organic phases were dried over MgSO4, filtered, and evaporated before the crude product was stirred in 10 ml MTBE. The product was filtered off, washed with water, and dried. 140 mg was dissolved in dry DMF and added dropwise to a mixture of 89 mg 2-Methylpyrazole-3-carboxylic acid in 3 ml dry DMF containing 334 mg HATU and 0.31 ml DIPEA, and the reaction mixture was stirred at ambient temperature for 16 h. The reaction mixture was evaporated before being purified by preparative RP-HPLC (Waters Sunfire Prep C18 OBD 5 μm 50×100 mm; 120 ml/min, H₂O (+0.1% TFA)/10% ACN to 10% H₂O (+0.1% TFA)/90%) to afford 4 mg of the title compound after freeze drying of the fractions containing pure product. LC/MS: m/z=507.1 [M+H]⁺; rt: 1.84 min (LC/MS-method A). ¹H NMR (400 MHz, DMSO-d₆) δ ppm 10.69 (br s, 1H), 8.36-8.43 (m, 2H), 8.23 (d, J=5.2 Hz, 1H), 7.97 (s, 1H), 7.47-7.50 (m, 1H), 7.01-7.04 (m, 2H) 4.89 (br t, J=8.2 Hz, 1H), 4.03 (s, 3H), 3.47 (br s, 2H), 3.26-3.32 (m, 2H), 2.35-2.45 (m, 2H), 0.72-0.99 (m, 3H), 0.10-0.50 (m, 8H).

### Example 32: (S)—N-(1,1-dicyclopropyl-3-oxo-3-((4-((4,4,4-trifluorobutanamido)-methyl)pyridin-2-yl)amino)propan-2-yl)-1-methyl-1H-pyrazole-5-carboxamide

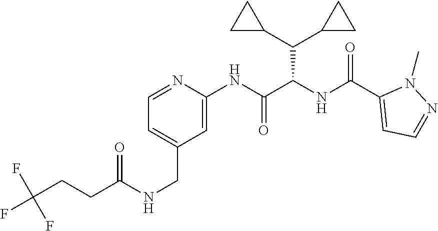 [SMILES: Cn1nccc1C(=O)N[C@H](C(=O)Nc1cc(CNC(=O)CCC(F)(F)F)ccn1)C(C1CC1)C1CC1]

Following the procedure described in Example 31, using tert-butyl (S)-(1,1-dicyclopropyl-3-oxo-3-((4-((4,4,4-trifluorobutanamido)methyl)pyridin-2-yl)amino)propan-2-yl)carbamate (Intermediate 31), 3.5 mg of the title compound was obtained. LC/MS: m/z=507.2 [M+H]⁺; rt: 1.86 min (LC/MS-method A). ¹H NMR (400 MHz, DMSO-d₆) δ ppm 10.78 (br s, 1H), 8.67 (br t, J=5.9, 1H), 8.43 (d, J=8.7 Hz, 1H), 8.25 (d, J=5.1 Hz, 1H), 7.94 (s, 1H), 7.48 (d, J=2.0 Hz, 1H), 7.00-7.04 (m, 2H), 4.90 (br t, J=7.8 Hz, 1H), 4.31 (d, J=5.9 Hz, 2H), 4.03 (s, 3H), 2.42-2.58 (m, 4H), 0.72-0.99 (m, 3H), 0.10-0.50 (m, 8H).

### Example 33: N—((S)-1,1-dicyclopropyl-3-((4-(((R)-4-methyl-2-oxoimidazolidin-1-yl)methyl)pyridin-2-yl)amino)-3-oxopropan-2-yl)-1-methyl-1H-pyrazole-5-carboxamide

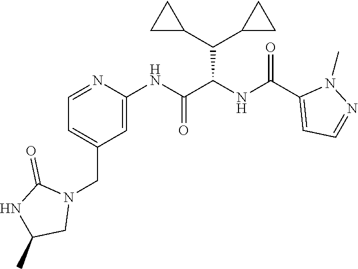 [SMILES: C[C@@H]1CN(Cc2ccnc(NC(=O)[C@@H](NC(=O)c3ccnn3C)C(C3CC3)C3CC3)c2)C(=O)N1]

21 mg of 2-Methylpyrazole-3-carboxylic acid was dissolved in 2 ml dry DMF and 80 mg HATU and 0.073 ml DIPEA were added, and the mixture stirred for 30 min at ambient temperature before addition of 50 mg of (S)-2-amino-3,3-dicyclopropyl-N-(4-(((R)-4-methyl-2-oxoimidazolidin-1-yl)methyl)pyridin-2-yl)propenamide HCl salt (Intermediate 32). The reaction mixture was stirred at ambient temperature for 16 h and evaporated before being purified by preparative RP-HPLC (Waters Sunfire Prep C18 OBD 5 μm 50×100 mm; 120 ml/min, H₂O (+0.1% TFA)/10% ACN to 10% H₂O (+0.1% TFA)/90%) to afford 5.5 mg of the title compound after freeze drying of the fractions containing pure product. LC/MS: m/z=466.2 [M+H]⁺; rt: 1.66 min (LC/MS-method A). ¹H NMR (400 MHz, DMSO-d₆) δ ppm 10.82 (br s, 1H), 8.43 (d, J=8.7 Hz, 1H), 8.27 (d, J=5.2 Hz, 1H), 7.95 (s, 1H), 7.47 (d, J=2.0 Hz, 1H), 6.99-7.06 (m, 2H), 6.67 (br s, 1H), 4.91 (br t, J=8.0, 1H), 4.32 (d, J=16.3, 1H), 4.22 (d, J=16.3, 1H), 4.03 (s, 3H), 3.61-3.70 (m, 1H), 3.39 (t, J=8.4 Hz, 1H), 2.80 (dd, J=8.8 Hz, 6.2 Hz, 1H), 1.11 (d, J=6.1 Hz, 3H), 0.72-0.99 (m, 3H), 0.10-0.50 (m, 8H).

### Example 34: 1-cyclopropyl-N—((S)-1,1-dicyclopropyl-3-((4-(((R)-4-methyl-2-oxoimidazolidin-1-yl)methyl)pyridin-2-yl)amino)-3-oxopropan-2-yl)-1H-pyrazole-5-carboxamide

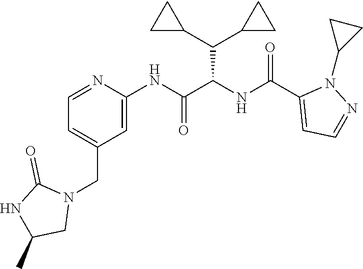 [SMILES: C[C@@H]1CN(Cc2ccnc(NC(=O)[C@@H](NC(=O)c3ccnn3C3CC3)C(C3CC3)C3CC3)c2)C(=O)N1]

Following the procedure described in Example 33, using 2-cyclopropylpyrazole-3-carboxylic, 10 mg of the title compound was obtained. LC/MS: m/z=492.2 [M+H]⁺; rt: 1.78 min (LC/MS-method A). ¹H NMR (400 MHz, DMSO-d₆) δ ppm 10.88 (br s, 1H), 8.47 (d, J=8.6 Hz, 1H), 8.28 (d, J=5.2 Hz, 1H), 7.95 (s, 1H), 7.43 (d, J=2.0 Hz, 1H), 7.03 (br d, J=5.1 Hz, 1H), 6.95 (d, J=2.0 Hz 1H), 6.51-6.80 (br s, 1H), 4.23 (br t, J=7.9 Hz, 1H), 4.39-4.46 (m, 1H), 4.32 (d, J=16.4, 1H), 4.23 (d, J=16.4, 1H), 3.66-2.72 (m, 1H), 3.40 (t, J=8.4 Hz, 1H), 2.78-2.83 (dd, J=8.6 Hz, 6.8 Hz, 1H), 0.73-1.15 (m, 10H), 0.12-0.51 (m, 8H).

### Example 35: N-(1-(4,4-difluorocyclohexyl)-2-((4-((1-methyl-6-oxo-1,6-dihydropyridin-2-yl)methyl)pyridin-2-yl)amino)-2-oxoethyl)-1-isopropyl-1H-pyrazole-5-carboxamide

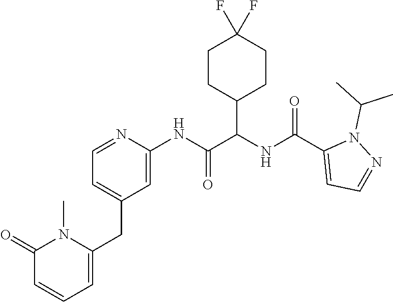 [SMILES: CC(C)n1nccc1C(=O)NC(C(=O)Nc1cc(Cc2cccc(=O)n2C)ccn1)C1CCC(F)(F)CC1]

Following the procedure described in Example 33 using 13 mg of 1-isopropyl-1H-pyrazole-5-carboxylic acid and 34 mg of 2-amino-2-(4,4-difluorocyclohexyl)-N-(4-((1-methyl-6-oxo-1,6-dihydropyridin-2-yl)methyl)pyridin-2-yl)acetamide (Intermediate 33), 14.5 mg of the title compound was obtained. LC/MS: m/z=527.3 [M+H]⁺; rt: 1.83 min (LC/MS-method A). ¹H NMR (400 MHz, DMSO-de) δ ppm 10.71 (s, 1H), 8.54 (d, J=8.2 Hz, 1H), 8.28-8.30 (m, 1H), 7.98 (s, 1H), 7.49 (d, J=1.9 Hz, 1H), 7.35 (dd, J=9.1 Hz, 6.8 Hz, 1H), 6.96 (dd, J=5.1 Hz, 1.3 Hz, 1H), 6.92 (d, J=2.0 Hz, 1H), 6.33 (dd, J=9.1 Hz, 1.1 Hz, 1H), 6.06 (dd, J=6.9 Hz, 1.0 Hz, 1H), 5.31-5.39 (m, 1H), 4.63 (t, J=8.3 Hz, 1H), 4.14 (s, 2H), ~3.30 (s, 3H, below water signal), 2.52-2.54 (m, 1H), 0.74-2.15 (m, 14H).

### Example 36: N-(1-(4,4-difluorocyclohexyl)-2-((4-((1-methyl-6-oxo-1,6-dihydro-pyridin-2-yl)methyl)pyridin-2-yl)amino)-2-oxoethyl)-1-ethyl-1H-pyrazole-5-carboxamide

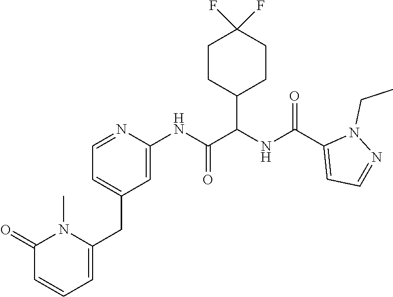 [SMILES: CCn1nccc1C(=O)NC(C(=O)Nc1cc(Cc2cccc(=O)n2C)ccn1)C1CCC(F)(F)CC1]

Following the procedure described in Example 33 using 12 mg 1-ethyl-1H-pyrazole-5-carboxylic acid and 34 mg 2-amino-2-(4,4-difluorocyclohexyl)-N-(4-((1-methyl-6-oxo-1,6-dihydropyridin-2-yl)methyl)pyridin-2-yl)acetamide (Intermediate 33), 27 mg of the title compound was obtained. LC/MS: m/z=513.3 [M+H]⁺; rt: 1.71 min (LC/MS-method A). ¹H NMR (400 MHz, DMSO-de) δ ppm 10.71 (s, 1H), 8.55 (d, J=8.3 Hz, 1H), 8.29 (d, J=5.1 Hz, 1H), 7.97 (s, 1H), 7.48 (d, J=2.0 Hz, 1H), 7.35 (dd, J=9.1 Hz, 6.8 Hz, 1H), 6.99 (d, J=2.0 Hz, 1H), 6.96 (dd, J=5.1 Hz, 1.3 Hz, 1H), 6.33 (dd, J=9.1 Hz, 1.1 Hz, 1H), 6.06 (dd, J=6.9 Hz, 1.0 Hz, 1H), 4.61-4.67 (m, 1H), 4.44 (q, J=7.1 Hz, 2H), 4.14 (s, 2H), ~3.30 (s, 3H, below water signal), 2.36-2.46 (m, 1H), 0.67-2.21 (m, 11H).

### Example 37: N-(1-(4,4-difluorocyclohexyl)-2-((4-((1-methyl-6-oxo-1,6-dihydropyridin-2-yl)methyl)pyridin-2-yl)amino)-2-oxoethyl)-1-methyl-1H-pyrazole-5-carboxamide DS1

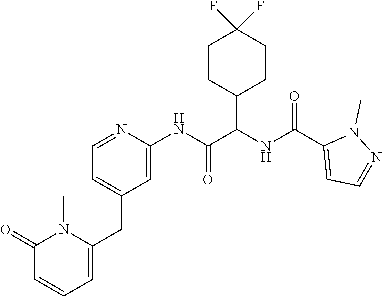 [SMILES: Cn1nccc1C(=O)NC(C(=O)Nc1cc(Cc2cccc(=O)n2C)ccn1)C1CCC(F)(F)CC1]

Following the procedure described in Example 33 using 11 mg of 2-Methylpyrazole-3-carboxylic acid and 34 mg of 2-amino-2-(4,4-difluorocyclohexyl)-N-(4-((1-methyl-6-oxo-1,6-dihydropyridin-2-yl)methyl)pyridin-2-yl)acetamide (Intermediate 33), 27 mg of the title compound was obtained. LC/MS: m/z=499.3 [M+H]⁺; rt: 1.60 min (LC/MS-method A). ¹H NMR (400 MHz, DMSO-de) δ ppm 10.72 (s, 1H), 8.55 (d, J=8.1 Hz, 1H), 8.29 (d, J=5.2 Hz, 1H), 7.97 (s, 1H), 7.46 (d, J=2.0 Hz, 1H), 7.35 (dd, J=9.1 Hz, 6.8 Hz, 1H), 7.03 (d, J=2.1 Hz, 1H), 6.96 (dd, J=5.1 Hz, 1.3 Hz, 1H), 6.33 (dd, J=9.1 Hz, 1.1 Hz, 1H), 6.06 (dd, J=6.9 Hz, 1.0 Hz, 1H), 4.61-4.67 (m, 1H), 4.13 (s, 2H), 4.01 (s, 3H), ~3.30 (s, 3H, below water signal), 2.51-2.54 (m, 1H), 0.67-2.21 (m, 8H).

### Example 38: N-((2S)-1,1-dicyclopropyl-3-((4-(cyclopropyl(4,4,4-trifluorobutanamido)methyl)pyridin-2-yl)amino)-3-oxopropan-2-yl)-1-methyl-1H-pyrazole-5-carboxamide DS1

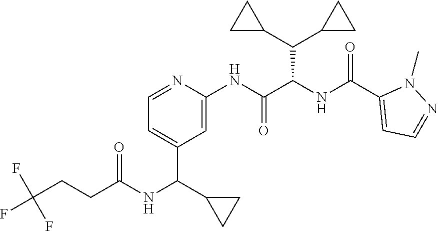 [SMILES: Cn1nccc1C(=O)N[C@H](C(=O)Nc1cc(C(NC(=O)CCC(F)(F)F)C2CC2)ccn1)C(C1CC1)C1CC1]

Following the procedure described in Example 33 using 13 mg of 2-methylpyrazole-3-carboxylic acid and 40 mg of N-((2-((S)-2-amino-3,3-dicyclopropylpropanamido)pyridin-4-yl)(cyclopropyl)methyl)-4,4,4-trifluorobutanamide (Intermediate 34 diastereomer 1), 6 mg of the title compound was obtained. LC/MS: m/z=547.2 [M+H]⁺; rt: 2.08 min (LC/MS-method A). ¹H NMR (400 MHz, DMSO-d₆) δ ppm 10.72 (s, 1H), 8.71-8.78 (m, 1H), 8.40 (d, J=8.6 Hz, 1H), 8.23-8.27 (m, 1H), 8.06 (s, 1H), 7.48 (d, J=2.0 Hz, 1H), 7.09-7.12 (m, 1H), 7.02-7.04 (m, 1H), 4.90 (br t, J=7.7 Hz, 1H), 4.14-4.21 (m, 1H), 4.03 (s, 3H), 2.41-2.47 (m, 2H), 0.72-1.26 (m, 6H), 0.12-0.56 (m, 12H).

### Example 39: 4-Cyclopropyl-N-((2S)-1,1-dicyclopropyl-3-((4-(cyclopropyl(4,4,4-trifluorobutanamido)methyl)pyridin-2-yl)amino)-3-oxopropan-2-yl)-1,2,5-oxadiazole-3-carboxamide DS1

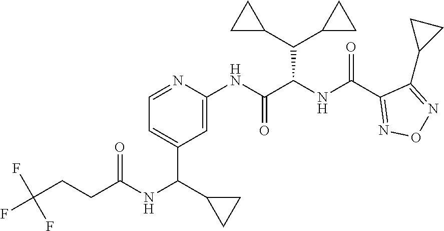 [SMILES: O=C(CCC(F)(F)F)NC(c1ccnc(NC(=O)[C@@H](NC(=O)c2nonc2C2CC2)C(C2CC2)C2CC2)c1)C1CC1]

15 mg 4-cyclopropyl-1,2,5-oxadiazole-3-carboxylic acid was dissolved in 3 ml DCM and 0.15 ml T3P (50% in EA), 0.05 ml DIPEA, and 40 mg N-((2-((S)-2-amino-3,3-dicyclopropylpropanamido)pyridin-4-yl)(cyclopropyl)methyl)-4,4,4-trifluorobutanamide (Intermediate 34 diastereomer 1) dissolved in DCM were added, and the mixture stirred at ambient temperature for 16 h. The reaction mixture was evaporated, and 15 mg of the title compound was obtained after purification by preparative RP-HPLC (Waters Sunfire Prep C18 OBD 5 μm 50×100 mm; 120 ml/min, H₂O (+0.1% TFA)/10% ACN to 10% H2O (+0.1% TFA)/90%) after freeze drying of the fractions containing pure product. LC/MS: m/z=575.2 [M+H]⁺; rt: 2.53 min (LC/MS-method A). ¹H NMR (400 MHz, DMSO-d₆) δ ppm 10.73 (s, 1H), 9.11-9.16 (m, 1H), 8.71-8.77 (m, 1H), 8.24-8.26 (m, 1H), 8.06-8.09 (m, 1H), 7.08-7.11 (m, 1H), 4.95-5.02 (m, 1H), 4.14-4.21 (m, 1H), 2.39-2.53 (m, 4H), 2.25-2.33 (m, 1H), 0.72-1.16 (m, 8H), 0.14-0.56 (m, 12H).

### Example 40: 4-Cyclopropyl-N-((2S)-1,1-dicyclopropyl-3-((4-(cyclopropyl(4,4,4-trifluorobutanamido)methyl)pyridin-2-yl)amino)-3-oxopropan-2-yl)-1,2,5-oxadiazole-3-carboxamide DS2

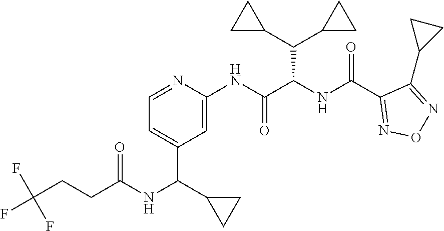 [SMILES: O=C(CCC(F)(F)F)NC(c1ccnc(NC(=O)[C@@H](NC(=O)c2nonc2C2CC2)C(C2CC2)C2CC2)c1)C1CC1]

Following the procedure described in Example 39 using 19 mg of 4-cyclopropyl-1,2,5-oxadiazole-3-carboxylic and 50 mg of N-((2-((S)-2-amino-3,3-dicyclopropyl-propanamido)pyridin-4-yl)(cyclopropyl)methyl)-4,4,4-trifluorobutanamide (Intermediate 34 diastereomer 2), 5 mg of the title compound was obtained. LC/MS: m/z=575.2 [M+H]⁺; rt: 2.53 min (LC/MS-method A). ¹H NMR (400 MHz, DMSO-de) δ ppm 10.77 (s, 1H), 9.12-9.17 (m, 1H), 8.71-8.78 (m, 1H), 8.24-8.26 (m, 1H), 8.05-8.09 (m, 1H), 7.09-7.13 (m, 1H), 4.95-5.02 (m, 1H), 4.14-4.22 (m, 1H), 2.39-2.53 (m, 4H), 2.25-2.33 (m, 1H), 0.72-1.16 (m, 8H), 0.14-0.56 (m, 12H).

### Example 41: N-((2S)-1,1-dicyclopropyl-3-((4-(cyclopropyl(4,4,4-trifluorobutan-amido)methyl)pyridin-2-yl)amino)-3-oxopropan-2-yl)-1-isopropyl-1H-pyrazole-5-carboxamide DS1

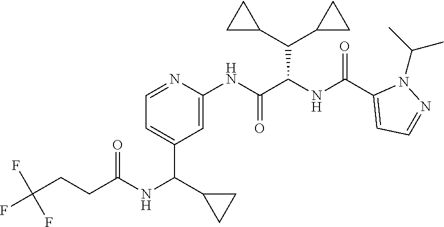 [SMILES: CC(C)n1nccc1C(=O)N[C@H](C(=O)Nc1cc(C(NC(=O)CCC(F)(F)F)C2CC2)ccn1)C(C1CC1)C1CC1]

Following the procedure described in Example 33, using 15 mg of 2-isopropylpyrazole-3-carboxylic acid and 40 mg of N-((2-((S)-2-amino-3,3-dicyclopropylpropanamido)pyridin-4-yl)(cyclopropyl)methyl)-4,4,4-trifluorobutanamide (Intermediate 34 diastereomer 1), 6 mg of the title compound was obtained. LC/MS: m/z=575.3 [M+H]⁺; rt: 2.29 min (LC/MS-method A). ¹H NMR (400 MHz, DMSO-d₆) δ ppm 10.71 (s, 1H), 8.71-8.77 (m, 1H), 8.41 (d, H=8.7 Hz, 1H), 8.25 (d, H=5.1 Hz, 1H), 8.03-8.08 (m, 1H), 7.51 (d, J=2.0, 1H), 7.09-7.13 (m, 1H), 6.90-6.92 (m, 1H), 5.34-5.42 (m, 1H), 4.89 (br t, J=7.8 Hz, 1H), 4.14-4.21 (m, 1H), 2.39-2.54 (m, 4H), 1.32-1.39 (m, 6H), 1.03-1.13 (m, 1H), 0.71-1.00 (m, 3H), 0.12-0.57 (m, 12H).

### Example 42: N-((2S)-1,1-dicyclopropyl-3-((4-(cyclopropyl(4,4,4-trifluorobutan-amido)methyl)pyridin-2-yl)amino)-3-oxopropan-2-yl)-1-methyl-1H-pyrazole-5-carboxamide DS2

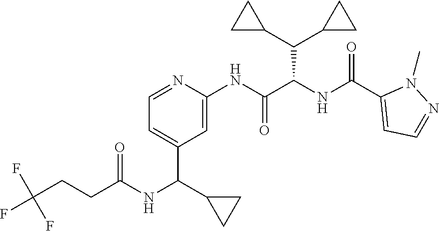 [SMILES: Cn1nccc1C(=O)N[C@H](C(=O)Nc1cc(C(NC(=O)CCC(F)(F)F)C2CC2)ccn1)C(C1CC1)C1CC1]

Following the procedure described in Example 33 using 16 mg of 2-methylpyrazole-3-carboxylic acid and 50 mg of N-((2-((S)-2-amino-3,3-dicyclopropylpropanamido)pyridin-4-yl)(cyclopropyl)methyl)-4,4,4-trifluorobutanamide (Intermediate 34, diastereomer 2), 6 mg of the title compound was obtained. LC/MS: m/z=547.3 [M+H]⁺; rt: 2.08 min (LC/MS-method A). ¹H NMR (400 MHz, DMSO-d₆) δ ppm 10.86 (s, 1H), 8.73-8.76 (m, 1H), 8.44 (d, H=8.6 Hz, 1H), 8.25-8.28 (m, 1H), 8.04 (s, 1H), 7.48 (d, J=2.0, 1H), 7.13-7.16 (m, 1H), 7.03-7.05 (m, 1H), 4.89 (br t, J=7.8 Hz, 1H), 4.15-4.22 (m, 1H), 4.03 (s, 3H), 2.41-2.47 (m, 2H), 0.72-1.26 (m, 6H), 0.12-0.56 (m, 12H).

### Example 43: N-((2S)-1,1-dicyclopropyl-3-((4-(cyclopropyl(4,4,4-trifluorobutanamido)methyl)pyridin-2-yl)amino)-3-oxopropan-2-yl)-1-isopropyl-1H-pyrazole-5-carboxamide DS2

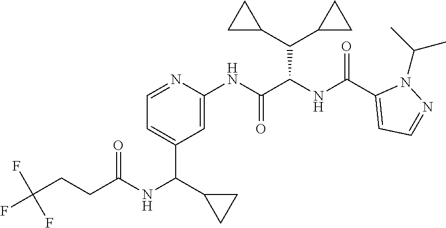 [SMILES: CC(C)n1nccc1C(=O)N[C@H](C(=O)Nc1cc(C(NC(=O)CCC(F)(F)F)C2CC2)ccn1)C(C1CC1)C1CC1]

Following the procedure described in Example 33 using 19 mg of 2-isopropylpyrazole-3-carboxylic acid and 50 mg of N-((2-((S)-2-amino-3,3-dicyclopropylpropanamido)pyridin-4-yl)(cyclopropyl)methyl)-4,4,4-trifluorobutanamide (Intermediate 34 diastereomer 2), 6 mg of the title compound was obtained. LC/MS: m/z=575.3 [M+H]⁺; rt: 2.30 min (LC/MS-method A). ¹H NMR (400 MHz, DMSO-d₆) δ ppm 10.78 (s, 1H), 8.72-8.78 (m, 1H), 8.42 (d, H=8.5 Hz, 1H), 8.26 (d, H=5.2 Hz, 1H), 8.03-8.06 (m, 1H), 7.51 (d, J=2.0, 1H), 7.11-7.15 (m, 1H), 6.90-6.92 (m, 1H), 5.34-5.42 (m, 1H), 4.89 (br t, J=7.8 Hz, 1H), 4.14-4.21 (m, 1H), 2.39-2.54 (m, 4H), 1.32-1.39 (m, 6H), 1.03-1.13 (m, 1H), 0.71-1.00 (m, 3H), 0.12-0.57 (m, 12H).

### Example 44: N-((2S)-1,1-dicyclopropyl-3-((4-(cyclopropyl(4,4,4-trifluorobutan-amido)methyl)pyridin-2-yl)amino)-3-oxopropan-2-yl)-3-ethylisoxazole-4-carboxamide DS2

Following the procedure described in Example 33 using 18 mg of 3-ethylisoxazole-4-carboxylic acid and 50 mg of N-((2-((S)-2-amino-3,3-dicyclopropylpropanamido)pyridin-4-yl)(cyclopropyl)methyl)-4,4,4-trifluorobutanamide (Intermediate 34 diastereomer 2), 5 mg of the title compound was obtained. LC/MS: m/z=562.3 [M+H]⁺; rt: 2.23 min (LC/MS-method A). ¹H NMR (400 MHz, DMSO-d₆) δ ppm 10.64 (s, 1H), 9.42 (s, 1H), 8.70-8.75 (m, 1H), 8.35-8.39 (m, 1H), 8.23-8.26 (m, 1H), 8.07 (s, 1H), 7.08-7.11 (m, 1H), 4.92 (br t, J=7.8, 1H), 4.14-4.22 (m, 1H), 2.85 (q, J=7.4 Hz, 2H), 2.39-2.54 (m, 4H), 1.18 (t, J=7.5 Hz, 3H), 1.05-1.13 (m, 1H), 0.80-1.00 (m, 2H), 0.64-0.73 (m, 1H), 0.14-0.56 (m, 12H).

### Example 45: Tert-butyl (S)-(1-(4,4-difluorocyclohexyl)-2-oxo-2-((4-((((2,2,2-trifluoroethyl)carbamoyl)oxy)methyl)pyridin-2-yl)amino)ethyl)carbamate

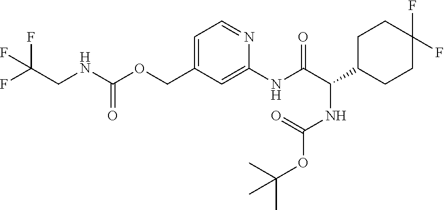 [SMILES: CC(C)(C)OC(=O)N[C@H](C(=O)Nc1cc(COC(=O)NCC(F)(F)F)ccn1)C1CCC(F)(F)CC1]

Under Ar, (2-aminopyridin-4-yl)methyl (2,2,2-trifluoroethyl)-carbamate (Intermediate 10, 8 mg) was dissolved in dry THE (1 ml), cooled to −78° C., and nBuLi in hexane (1.6 M, 64 μl) was added with stirring. After stirring for 15 min methyl (S)-2-((tert-butoxycarbonyl)amino)-2-(4,4-difluorocyclohexyl)acetate (Intermediate 13, 10 mg) dissolved in THE (0.1 ml) was added. After 15 min, the cooling bath was removed, and stirring was continued for 1 h. Then saturated ammonium chloride solution was added, and the mixture was left aside over the weekend. The aqueous phase was extracted with EA (2×). The combined organic phases were dried (Chem Elut cartridge) and concentrated in vacuo. The residue was purified by preparative HPLC (Agilent Prep C18, 30 mm×100 mm, 5 μm; 40 ml/min, from 97% H₂O/3% ACN to 10% H₂O/90% ACN in 12.5 min). The pure product containing fractions were combined, the ACN was partly removed and the residue freeze dried to yield 1.2 mg of the title compound. LC/MS: m/z=525.2 [M+H]⁺; rt: 2.23 min (LC/MS-method A). ¹H NMR (400 MHz, DMSO-d₆) δ ppm 10.51 (s, 1H), 8.30 (d, J=5.0 Hz, 1H), 8.24 (t, J=6.2 Hz, 1H), 8.1 (s, 1H), 7.12 (d, J=8.3 Hz, 1H), 7.05 (d, J=4.8 Hz, 1H), 5.12 (s, 1H), 4.18 (t, J=8.1 Hz, 1H), 3.77-3.87 (m, 1H), 1.95-2.07 (m, 2H), 1.56-1.85 (m, 6H), 1.27-1.43 (m, 12H).

### Example 46: 4-cyclopropyl-N-(2-((4-(cyclopropyl(4,4,4-trifluorobutan-amido)methyl)pyridin-2-yl)amino)-1-(4,4-difluorocyclohexyl)-2-oxoethyl)-1,2,5-oxadiazole-3-carboxamide

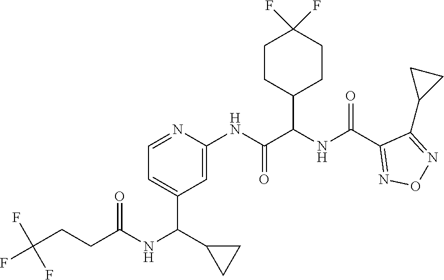 [SMILES: O=C(CCC(F)(F)F)NC(c1ccnc(NC(=O)C(NC(=O)c2nonc2C2CC2)C2CCC(F)(F)CC2)c1)C1CC1]

Following the procedure escribe in Example 39 using 20 mg of 4-cyclopropyl-1,2,5-oxadiazole-3-carboxylic and 55 mg of N-((2-(2-amino-2-(4,4-difluorocyclohexyl)-acetamido)pyridin-4-yl)(cyclopropyl)methyl)-4,4,4-trifluorobutanamide diastereomer 1 (Intermediate 35), 15 mg of the title compound was obtained. LC/MS: m/z=599.2 [M+H]⁺; rt: 2.41 min (LC/MS-method A). ¹H NMR (400 MHz, DMSO-d₆) δ ppm 10.79 (d, J=5.0 Hz, 1H), 9.30 (d, J=8.1 Hz, 1H), 8.73 (d, J=7.7 Hz, 1H), 8.26-8.28 (m, 1H), 8.10 (s, 1H), 7.10-7.13 (m, 1H), 4.70-4.77 (m, 1H), 4.13-4.19 (m, 1H), 2.40-2.48 (m, 3H), 2.24-2.31 (m, 1H), 1.98-2.10 (m, 3H), 1.65-1.92 (m, 5H), 1.46-1.56 (m, 1H), 1.31-1.41 (m, 1H), 1.05-1.15 (m, 3H), 0.96-1.00 (m, 2H), 0.50-0.56 (m, 2H), 0.31-0.43 (m, 2H).

### Example 47: N-(2-((4-(cyclopropyl(4,4,4-trifluorobutanamido)methyl)pyridin-2-yl)amino)-1-(4,4-difluorocyclohexyl)-2-oxoethyl)-1-methyl-1H-pyrazole-5-carboxamide

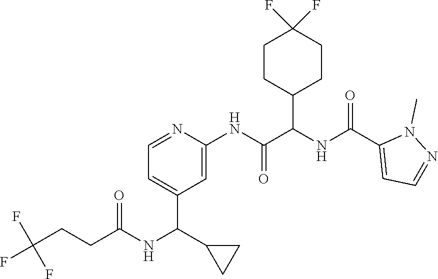 [SMILES: Cn1nccc1C(=O)NC(C(=O)Nc1cc(C(NC(=O)CCC(F)(F)F)C2CC2)ccn1)C1CCC(F)(F)CC1]

Following the procedure described in Example 33 using 16 mg of 2-methylpyrazole-3-carboxylic acid and 55 mg of N-((2-(2-amino-2-(4,4-difluorocyclohexyl)-acetamido)pyridin-4-yl)(cyclopropyl)methyl)-4,4,4-trifluorobutanamide diastereomer 1 (Intermediate 35), 15 mg of the title compound was obtained. LC/MS: m/z=571.2 [M+H]⁺; rt: 2.00 min (LC/MS-method A). ¹H NMR (400 MHz, DMSO-d₆) δ ppm 10.81 (br s, 1H), 8.74 (d, J=7.7 Hz, 1H), 8.59 (d, J=7.9 Hz, 1H), 8.26-8.28 (m, 1H), 8.08 (s, 1H), 7.47 (d, J=2.0 Hz, 1H), 7.12-7.15 (m, 1H), 7.04 (t, J=2.0, 1H), 4.59-4.66 (m, 1H), 4.13-4.19 (m, 1H), 4.02 (s, 3H), 2.39-2.49 (m, 4H), 1.65-2.11 (m, 7H), 1.43-1.54 (m, 1H), 1.29-1.41 (m, 1H), 1.04-1.13 (m, 1H), 0.49-0.55 (m, 2H), 0.31-0.43 (m, 2H).

### Example 48: N-(2-((4-(cyclopropyl(4,4,4-trifluorobutanamido)methyl)pyridin-2-yl)amino)-1-(4,4-difluorocyclohexyl)-2-oxoethyl)-1-isopropyl-1H-pyrazole-5-carboxamide

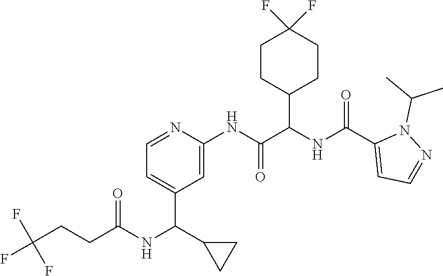 [SMILES: CC(C)n1nccc1C(=O)NC(C(=O)Nc1cc(C(NC(=O)CCC(F)(F)F)C2CC2)ccn1)C1CCC(F)(F)CC1]

Following the procedure described in Example 33 using 20 mg of 2-isopropylpyrazole-3-carboxylic acid and 55 mg of N-((2-(2-amino-2-(4,4-difluoro-cyclohexyl)acetamido)pyridin-4-yl)(cyclopropyl)methyl)-4,4,4-trifluorobutanamide diastereomer 1 (Intermediate 35), 14 mg of the title compound was obtained. LC/MS: m/z=599.2 [M+H]⁺; rt: 2.21 min (LC/MS-method A). ¹H NMR (400 MHz, DMSO-d₆) δ ppm 10.82 (s, 1H), 8.72-8.77 (m, 1H), 8.56 (d, J=7.8 Hz, 1H), 8.27 (d, J=5.3 Hz, 1H), 8.07 (s, 1H), 7.50 (d, J=1.9 Hz, 1H), 7.13-7.16 (m, 1H), 6.93-6.95 (m, 1H), 5.36 (sep, J=6.6 Hz, 1H), 4.57-4.64 (m, 1H), 4.12-4.19 (m, 1H), 2.38-2.54 (m, 4H), 1.65-2.11 (m, 7H), 1.42-1.54 (m, 1H), 1.30-1.42 (m, 7H), 1.04-1.14 (m, 1H), 0.48-0.57 (m, 2H), 0.31-0.44 (m, 2H).

### Example 49: N-(2-((4-(cyclopropyl(4,4,4-trifluorobutanamido)methyl)pyridin-2-yl)amino)-1-(4,4-difluorocyclohexyl)-2-oxoethyl)-3-ethylisoxazole-4-carboxamide

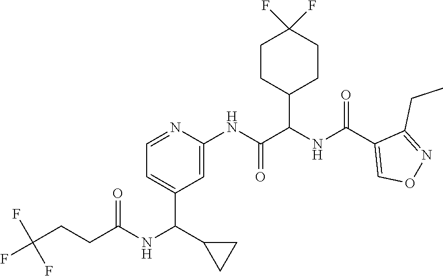 [SMILES: CCc1nocc1C(=O)NC(C(=O)Nc1cc(C(NC(=O)CCC(F)(F)F)C2CC2)ccn1)C1CCC(F)(F)CC1]

Following the procedure described in Example 33 using 18 mg of 3-ethylisoxazole-4-carboxylic acid and 55 mg of N-((2-(2-amino-2-(4,4-difluoro-cyclohexyl)acetamido)pyridin-4-yl)(cyclopropyl)methyl)-4,4,4-trifluorobutanamide diastereomer 1 (Intermediate 35), 16 mg of the title compound was obtained. LC/MS: m/z=586.2 [M+H]⁺; rt: 2.16 min (LC/MS-method A). ¹H NMR (400 MHz, DMSO-de) δ ppm 10.75 (br d, J=3.1 Hz, 1H), 9.41 (s, 1H), 8.73 (dd, J=7.6, 2.2 Hz, 1H), 8.54 (br d, J=7.7 Hz, 1H), 8.26 (br d, J=5.0 Hz, 1H), 8.09 (s, 1H), 7.10-7.13 (m, 1H), 4.62-4.70 (m, 1H), 4.12-4.19 (m, 1H), 2.83 (q, J=7.5 Hz, 2H), 2.37-2.54 (m, 4H), 1.64-2.12 (m, 7H), 1.31-1.54 (m, 2H), 1.04-1.20 (m, 4H), 0.48-0.57 (m, 2H), 0.31-0.44 (m, 2H).

### Example 50: Tert-butyl ((S)-2-((5-fluoro-4-(((S)-2-oxo-4-(trifluoromethyl)-imidazolidin-1-yl)methyl)pyridin-2-yl)amino)-1-((1r,4S)-4-methylcyclohexyl)-2-oxoethyl)carbamate

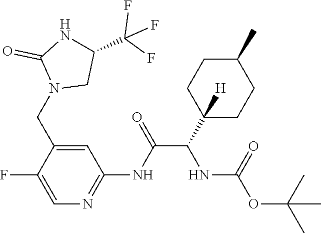

Tert-butyl ((S)-2-((4-((((S)-2-amino-3,3,3-trifluoropropyl)amino)-methyl)-5-fluoro-pyridin-2-yl)amino)-1-((1r,4S)-4-methylcyclohexyl)-2-oxoethyl)carbamate (Intermediate 14; 404 mg) was dissolved in dry THE (18 ml) under Ar at RT. After heating for 5 min at 65° C. CDI (389 mg) was added. After stirring for 4 h at 65° C. and standing overnight at RT, saturated NaHCO₃ solution was added and the aqueous mixture was extracted with DCM (3×). The combined organic phases were dried over sodium sulphate, filtered, and concentrated in vacuo. The residue was purified by silica gel chromatography (silica gel 24 g, DCM/EtOH: 100:0 to 90:10 in 40 min). The pure compound containing fractions were combined and concentrated in vacuo. The residue was dissolved in ACN/water and freeze dried to yield 343 mg of the title compound. LC/MS: m/z=532.2 [M+H]⁺; rt: 2.40 min (LC/MS-method A). ¹H NMR (400 MHz, DMSO-d₆) δ ppm 10.43 (s, 1H), 8.29 (d, J=1.0 Hz, 1H), 8.01 (d, J=5.4 Hz, 1H), 7.69 (d, J=2.0 Hz, 1H), 6.90 (d, J=8.3 Hz, 1H), 4.36-4.52 (m, 2H), 4.28 (d, J=16.3 Hz, 1H), 4.05 (t, J=7.7 Hz, 1H), 3.62 (t, J=9.9 Hz, 1H), 3.33-3.39 (m, 1H), 9.64-1.74 (m, 17H), 0.75-0.88 (m, 5H).

### Example 51: tert-butyl ((S)-2-((4-((1,4-dimethyl-1H-pyrazol-5-yl)methyl)pyridin-2-yl)amino)-1-((1r,4S)-4-methylcyclohexyl)-2-oxoethyl)carbamate

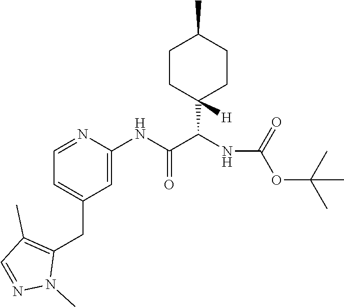 [SMILES: Cc1cnn(C)c1Cc1ccnc(NC(=O)[C@@H](NC(=O)OC(C)(C)C)[C@H]2CC[C@H](C)CC2)c1]

163 mg of (R)-2-((tert-butoxycarbonyl)amino)-2-((1r,4R)-4-methylcyclohexyl)-acetic acid was dissolved in dry DMF, and 252 mg HATU and 0.21 ml DIPEA were added, and the mixture stirred at ambient temperature for 30 min before addition of 122 mg of 4-((1,4-dimethyl-1H-pyrazol-5-yl)methyl)pyridin-2-amine (Intermediate 36). The mixture was heated to 50° C. for 16 h before evaporation to dryness, and the residue was purified by FC on silica gel using and EA:heptane gradient to give the 64 mg of the title product. LC/MS: m/z=456.3 [M+H]⁺; rt: 2.44 min (LC/MS-method A). ¹H NMR (400 MHz, DMSO-de) 8 ppm 10.30 (s, 1H), 8.20 (d, J=5.1 Hz, 1H), 7.92 (s, 1H), 7.21 (s, 1H), 6.90 (br d, J=8.7 Hz, 1H), 6.81 (dd, J=5.0 Hz, 0.1 Hz, 1H), 4.00-4.07 (m, 3H), 3.63 (s, 3H), 1.96 (s, 3H), 0.96-1.70 (m, 17H), 0.76-0.83 (m, 5H).

### Example 52: N-(1-(4,4-difluorocyclohexyl)-2-((4-((1-methyl-6-oxo-1,6-dihydro-pyridin-3-yl)methyl)pyridin-2-yl)amino)-2-oxoethyl)-1-methyl-1H-pyrazole-5-carboxamide

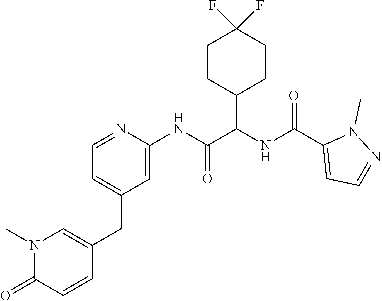 [SMILES: Cn1nccc1C(=O)NC(C(=O)Nc1cc(Cc2ccc(=O)n(C)c2)ccn1)C1CCC(F)(F)CC1]

Following the procedure described in Example 33 using 12 mg of 2-methylpyrazole-3-carboxylic acid and 38 mg of 2-amino-2-(4,4-difluorocyclohexyl)-N-(4-((1-methyl-6-oxo-1,6-dihydropyridin-3-yl)methyl)pyridin-2-yl)acetamide (Intermediate 37), 2.7 mg of the title compound was obtained. LC/MS: m/z=499.3 [M+H]⁺; rt: 1.57 min (LC/MS-method A). ¹H NMR (400 MHz, DMSO-d₆) δ ppm 10.68 (s, 1H), 8.55 (d, J=8.0 Hz, 1H), 8.23 (d, J=5.1 Hz, 1H), 7.96 (s, 1H), 7.64 (d, J=2.3 Hz, 1H), 7.47 (d, J=2.1 Hz, 1H), 7.27 (dd, J=9.3, 2.6 Hz, 1H), 7.03 (d, J=2.1 Hz, 1H), 7.00 (dd, J=5.1 Hz, 1.4 Hz, 1H), 6.34 (d, J=9.31 Hz, 1H), 4.63 (t, J=8.3 Hz, 1H), 4.01 (s, 3H), 3.70 (s, 2H), 3.49 (s, 3H), 1.94-2.11 (m, 3H), 1.62-1.92 (m, 4H), 1.41-1.53 (m, 1H), 1.28-1.39 (m, 1H).

### Example 53: N-(1-(4,4-difluorocyclohexyl)-2-((4-((1,4-dimethyl-1H-pyrazol-5-yl)methyl)pyridin-2-yl)amino)-2-oxoethyl)-1-methyl-1H-pyrazole-5-carboxamide

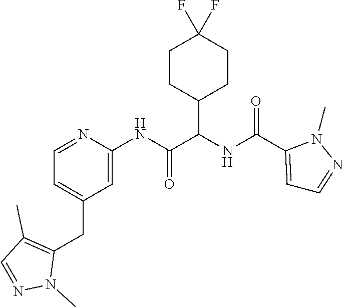 [SMILES: Cc1cnn(C)c1Cc1ccnc(NC(=O)C(NC(=O)c2ccnn2C)C2CCC(F)(F)CC2)c1]

Following the procedure described in Example 33 using 16 mg of 2-methylpyrazole-3-carboxylic acid and 49 mg of 2-amino-2-(4,4-difluorocyclohexyl)-N-(4-((1,4-dimethyl-1H-pyrazol-5-yl)methyl)pyridin-2-yl)acetamide (Intermediate 38), 8.1 mg of the title compound was obtained. LC/MS: m/z=484.2 [M−H]⁺; rt: 1.84 min (LC/MS-method A). ¹H NMR (400 MHz, DMSO-d₆) δ ppm 10.69 (s, 1H), 8.55 (d, J=8.0 Hz, 1H), 8.23 (d, J=5.1 Hz, 1H), 7.91 (s, 1H), 7.64 (d, J=2.1 Hz, 1H), 7.21 (s, 1H), 7.04 (d, J=2.1 Hz, 1H), 6.87 (dd, J=5.2 Hz, 1.4 Hz, 1H), 4.64 (t, J=8.4 Hz, 1H), 4.04 (s, 2H), 4.01 (s, 3H), 3.63 (s, 3H), 1.60-2.10 (m, 10H), 1.29-1.52 (m, 2H).

### Example 54: N-(1-(4,4-difluorocyclohexyl)-2-((4-((1-methyl-2-oxo-1,2-dihydro-pyridin-4-yl)methyl)pyridin-2-yl)amino)-2-oxoethyl)-1-methyl-1H-pyrazole-5-carboxamide

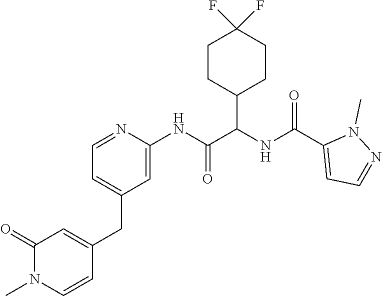 [SMILES: Cn1nccc1C(=O)NC(C(=O)Nc1cc(Cc2ccn(C)c(=O)c2)ccn1)C1CCC(F)(F)CC1]

Following the procedure describe in Example 33 using 18 mg of 2-methylpyrazole-3-carboxylic acid and 55 mg of 2-amino-2-(4,4-difluorocyclohexyl)-N-(4-((1-methyl-2-oxo-1,2-dihydropyridin-4-yl)methyl)pyridin-2-yl)acetamide (Intermediate 39), 9.6 mg of the title compound was obtained. LC/MS: m/z=499.3 [M−H]+; rt: 1.58 min (LC/MS-method A). ¹H NMR (400 MHz, DMSO-d₆) δ ppm 10.69 (s, 1H), 8.54 (d, J=7.9 Hz, 1H), 8.25 (d, J=5.1 Hz, 1H), 7.98 (s, 1H), 7.58 (d, J=6.9 Hz, 1H), 7.46 (d, J=2.1 Hz, 1H), 7.02-7.05 (m, 2H), 6.25 (d, J=1.1 Hz, 1H), 6.04 (dd, J=7.0 Hz, 9.1 Hz, 1H), 6.42 (t, J=8.4 Hz, 1H), 4.01 (s, 3H), 3.78 (s, 2H), 3.35 (s, 3H), 1.62-2.11 (m, 7H), 1.28-1.53 (m, 2H).

### Example 55: N—((S)-2-((4-((1,4-dimethyl-1H-pyrazol-5-yl)methyl)pyridin-2-yl)amino)-1-((1r,4S)-4-methylcyclohexyl)-2-oxoethyl)-1-isopropyl-1H-pyrazole-5-carboxamide

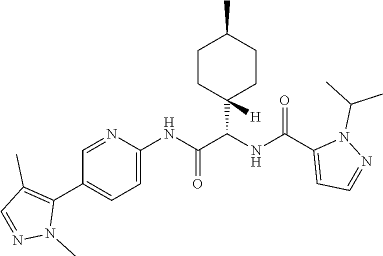 [SMILES: Cc1cnn(C)c1-c1ccc(NC(=O)[C@@H](NC(=O)c2ccnn2C(C)C)[C@H]2CC[C@H](C)CC2)nc1]

Following the procedure described in Example 33 using 20 mg of 2-isopropylpyrazole-3-carboxylic acid and 46 mg of (S)-2-amino-N-(4-((1,4-dimethyl-1H-pyrazol-5-yl)methyl)pyridin-2-yl)-2-((1r,4S)-4-methylcyclohexyl)acetamide (Intermediate 40), 30.3 mg of the title compound was obtained. LC/MS: m/z=492.4 [M+H]⁺; rt: 2.34 min (LC/MS-method A). ¹H NMR (400 MHz, DMSO-d₆) δ ppm 10.54 (s, 1H), 8.41 (d, J=8.1 Hz, 1H), 8.22 (d, J=5.1 Hz, 1H), 7.92 (s, 1H), 7.49 (d, J=1.9 Hz, 1H), 7.20 (s, 1H), 6.92 (d, J=2.0 Hz, 1H), 6.84 (dd, J=5.1, 1.4 Hz, 1H), 5.36 (sep, J=6.6 Hz, 1H), 4.49 (t, J=8.2 Hz, 1H), 4.03 (s, 2H), 3.63 (s, 3H), 1.96 (s, 3H), 1.51-183 (m, 5H), 1.34 (dd, J=10.0 Hz, 6.6 Hz, 6H), 1.20-1.28 (m, 2H), 1.00-1.12 (m, 1H), 0.79-0.91 (m, 5H).

### Example 56: (1R,2S,5S)—N-((2S)-1,1-dicyclopropyl-3-((4-(cyclopropyl(4,4,4-trifluoro-butanamido)methyl)pyridin-2-yl)amino)-3-oxopropan-2-yl)-3,6,6-trimethyl-3-azabicyclo[3.1.0]hexane-2-carboxamide DS2

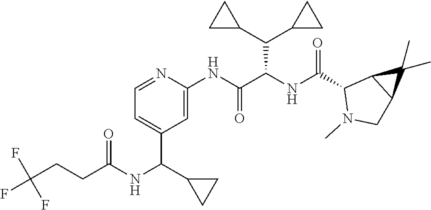 [SMILES: CN1C[C@H]2[C@@H]([C@H]1C(=O)N[C@H](C(=O)Nc1cc(C(NC(=O)CCC(F)(F)F)C3CC3)ccn1)C(C1CC1)C1CC1)C2(C)C]

Following the procedure from Example 39, using 60 mg of N-((2-((S)-2-amino-3,3-dicyclopropylpropanamido)pyridin-4-yl)(cyclopropyl)methyl)-4,4,4-trifluorobutanamide (Intermediate 34 diastereomer 2) and 25 mg of (1R,2S,5S)-3,6,6-trimethyl-3-azabicyclo[3.1.0]hexane-2-carboxylic acid, 8 mg of the title compound was obtained. LC/MS: m/z=590.4 [M+H]⁺; rt: 1.68 min (LC/MS-method A). ¹H NMR (400 MHz, DMSO-de) δ ppm 10.79 (s, 1H), 9.17 (d, J=8.7 Hz, 1H), 8.74 (d, J=7.8 Hz, 1H), 8.25 (d, J=5.2 Hz, 1H), 8.04 (s, 1H), 7.12 (dd, J=5.2 Hz, 1.2 Hz, 1H), 4.93-4.99 (m, 1H), 4.16-4.22 (m, 1H), 3.84-3.91 (m, 1H), 2.76-2.83 (m, 1H), 2.62-2.66 (m, 3H), 2.38-2.54 (m, 4H), 1.68-1.83 (m, 2H), 1.44-1.51 (m, 2H), 1.24 (s, 3H), 1.09 (s, 3H), 0.91-0.97 (m, 3H), 0.05-0.69 (m, 12H).

### Example 57: (5-Methylisoxazol-3-v)methyl ((S)-1-(4,4-difluorocyclohexyl)-2-oxo-2-((4-(((S)-2-oxo-4-(trifluoromethyl)imidazolidin-1-VI)methyl)pyridin-2-yl)amino)ethyl)carbamate

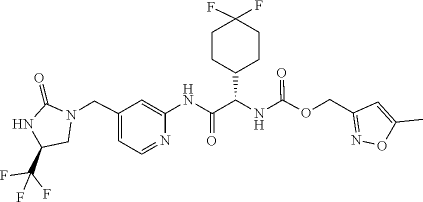 [SMILES: Cc1cc(COC(=O)N[C@H](C(=O)Nc2cc(CN3C[C@@H](C(F)(F)F)NC3=O)ccn2)C2CCC(F)(F)CC2)no1]

(S)-2-(4,4-Difluorocyclohexyl)-2-((((5-methylisoxazol-3-yl)methoxy)carbonyl)-amino)acetic acid (Intermediate 16, 10 mg) was dissolved in DCM, and TBTU (22 mg) and DIPEA (26 μl) were added with stirring. After 30 min, (S)-1-((2-aminopyridin-4-yl)methyl)-4-(trifluoromethyl)imidazolidin-2-one (Intermediate 15, 8 mg) suspended in DCM (0.5 ml) was added and the mixture was heated to reflux. After 16 h the mixture was cooled, and poured onto saturated NaHCO₃-solution. The aqueous phase was extracted with DCM (2×). The combined organic phases were dried (Chem Elut cartridge), and concentrated in vacuo. The residue was purified by preparative HPLC (Agilent Prep C18, 30 mm×100 mm, 5 μm; 40 ml/min, from 90% H₂O/10% ACN to 10% H₂O/90% ACN in 8 min). The pure product containing fractions were combined, the ACN was partly removed, and the residue freeze dried to yield 3 mg of the title compound. LC/MS: m/z=575.2 [M+H]⁺; rt: 1.87 min (LC/MS-method A). ¹H NMR (400 MHz, DMSO-de) 8 ppm 10.58 (s, 1H), 8.30 (d, J=5.1 Hz, 1H), 7.98 (br s, 1H), 7.73 (d, J=8.4 Hz, 1H), 7.66 (s, 1H), 6.95 (d, J=5.1 Hz, 1H), 6.20 (s, 1H), 5.03 (s, 2H), 4.20-4.46 (m, 4H), 3.55-3.63 (m, 1H), 3.32-3.36 (m, 1H), 2.38 (s, 3H), 1.22-2.07 (m, 9H).

### Example 58: 4-Cyclopropyl-N-((1S)-2-((4-(2-methoxy-1-((S)-2-oxo-4-(trifluoro-methyl)imidazolidin-1-yl)ethyl)pyridin-2-yl)amino)-1-((1r,4S)-4-methylcyclohexyl)-2-oxoethyl)-1,2,5-oxadiazole-3-carboxamide (DS2)

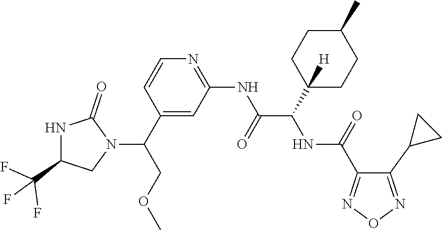

Following the procedure of Example 2, using (2S)-2-amino-N-(4-((R or S)-2-methoxy-1-((S)-2-oxo-4-(trifluoromethyl)imidazolidin-1-yl)ethyl)pyridin-2-yl)-2-((1r,4S)-4-methylcyclohexyl)acetamide hydrochloride salt (Intermediate 18 DS2; 50 mg), 37 mg of the title compound was obtained. LC/MS: m/z=594.4 [M+H]⁺; rt: 2.48 min (LC/MS-method A). ¹H NMR (400 MHz, DMSO-de) δ ppm 10.72 (s, 1H), 9.16 (d, J=8.2 Hz, 1H), 8.30 (d, J=5.3 Hz, 1H), 8.01 (s, 1H), 7.63 (d, J=1.8 Hz, 1H), 7.01 (dd, J=5.2 Hz, 0.6 Hz, 1H), 5.01 (dd, J=8.1, 5.1 Hz, 1H), 4.62 (t, J=7.9 Hz, 1H), 4.38-4.49 (m, 1H), 3.71-3.87 (m, 3H), 3.27-3.31 (m, 4H), 2.22-2.30 (m, 1H), 1.56-1.85 (m, 5H), 0.80-1.32 (m, 12H).

### Example 59: 4-Cyclopropyl-N-((1S)-2-((4-(2-methoxy-1-((S)-2-oxo-4-(trifluoro-methyl)imidazolidin-1-yl)ethyl)pyridin-2-yl)amino)-1-((1r,4S)-4-methylcyclohexyl)-2-oxoethyl)-1,2,5-oxadiazole-3-carboxamide (DS1)

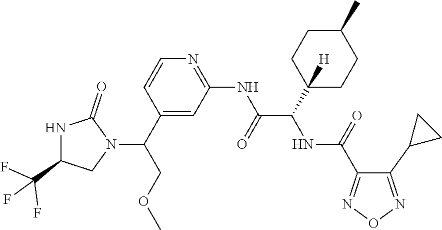

Following the procedure of Example 2, using (2S)-2-amino-N-(4-((R or S)-2-methoxy-1-((S)-2-oxo-4-(trifluoromethyl)imidazolidin-1-yl)ethyl)pyridin-2-yl)-2-((1r,4S)-4-methylcyclohexyl)acetamide hydrochloride salt (Intermediate 19 DS1; 50 mg) 38 mg of the title compound was obtained. LC/MS: m/z=594.4 [M+H]⁺; rt: 2.55 min (LC/MS-method A). ¹H NMR (400 MHz, DMSO-d₆) δ ppm 10.75 (s, 1H), 9.16 (d, J=8.1 Hz, 1H), 8.31 (d, J=5.1 Hz, 1H), 8.05 (s, 1H), 7.59 (d, J=2.20 Hz, 1H), 7.08 (dd, J=5.2, 1.1 Hz, 1H), 4.99 (dd, J=7.9, 5.9 Hz, 1H), 4.62 (t, J=7.9 Hz, 1H), 4.34-4.44 (m, 1H), 3.73-3.85 (m, 2H), 3.46-3.56 (m, 2H), 3.29 (s, 3H), 2.23-2.31 (m, 1H), 1.56-1.86 (m, 5H), 0.80-1.33 (m, 12H).

### Example 60: (1,5-Dimethyl-1H-pyrazol-3-yl)methyl ((S)-1-(4,4-difluorocyclohexyl)-2-oxo-2-((4-(((S)-2-oxo-4-(trifluoromethyl)imidazolidin-1-yl)methyl)pyridin-2-yl)amino)ethyl)carbamate

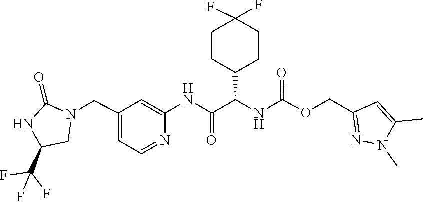 [SMILES: Cc1cc(COC(=O)N[C@H](C(=O)Nc2cc(CN3C[C@@H](C(F)(F)F)NC3=O)ccn2)C2CCC(F)(F)CC2)nn1C]

(S)-2-(4,4-Difluorocyclohexyl)-2-(((1,5-dimethyl-1H-pyrazol-3-yl)methoxy)-carbonyl)amino)acetic acid (Intermediate 20, 9 mg) was dissolved in DCM (0.5 ml), and DMAP (7 mg) and EDC (10 mg) were added with stirring. After 5 min, (S)-1-((2-aminopyridin-4-yl)methyl)-4-(trifluoromethyl)imidazolidin-2-one (Intermediate 15, 7 mg) was added. After 4 h, an additional equivalent of DMAP and EDC were added. After standing overnight, the mixture was poured onto saturated NaHCO₃-solution. The aqueous phase was extracted with DCM (2×). The combined organic phases were dried with magnesium sulphate, filtered, and concentrated in vacuo. The residue was purified by preparative HPLC (Agilent Prep C18, 30 mm×100 mm, 5 μm; 40 ml/min, from 97% H₂O/3% ACN to 10% H₂O/90% ACN in 12.5 min). The product containing fractions were combined, the ACN was partly removed, and the residue freeze dried. The residue was further purified by silica gel chromatography (Silica gel 4 g, DCM/EtOH: 100:0 for 5 min, 100:0 to 50:50 in 12 min, 50:50 for 5 min). The pure compound containing fractions were combined and concentrated in vacuo. The residue was dissolved in ACN/water and freeze dried to yield 2 mg of the title compound. LC/MS: m/z=588.2 [M+H]⁺; rt: 1.80 min (LC/MS-method A). ¹H NMR (400 MHz, DMSO-d₆) δ ppm 10.56 (d, J=4.0 Hz, 1H), 8.28 (d, J=5.0 Hz, 1H), 7.96-7.99 (m, 1H), 7.61-7.67 (m, 2H), 6.93-6.96 (m, 1H), 6.05 (s, 1H), 5.01 (s, 2H), 4.19-4.46 (m, 3H), 3.55-3.74 (m, 5H), 3.33-3.36 (m, 1H), 2.09 (s, 3H), 1.93-2.06 (m, 2H), 1.56-1.87 (m, 5H), 1.25-1.47 (m, 2H).

Using (S)-2-amino-N-(5-fluoro-4-(((S)-2-oxo-4-(trifluoromethyl)imidazolidin-1-yl)-methyl)pyridin-2-yl)-2-((1r,4S)-4-methylcyclohexyl)acetamide hydrochloride salt (Intermediate 21) as starting material, and 4 h reaction time analogously to Example 2, were prepared:

| Ex. | Acid | Structure | Name | Yield |
|---|---|---|---|---|
| 61 | | | 4-cyclopropyl-N-((S)-2- | 25 mg, |
| | | | ((5-fluoro-4-(((S)-2-oxo-4- | 73% |
| | | | (trifluoromethyl)imidazolidin- | |
| | | | 1-yl)methyl)pyridin-2- | |
| | | | yl)amino)-1-((1r,4S)-4- | |
| | | | methylcyclohexyl)-2-oxo- | |
| | | | ethyl)-1,2,5-oxadiazole-3- | |
| | | | carboxamide | |
| 62 | | | N-((S)-2-((5-fluoro-4-(((S)- | 16 mg, |
| | | | 2-oxo-4-(trifluoromethyl)- | 49% |
| | | | imidazolidin-1-yl)methyl)- | |
| | | | pyridin-2-yl)amino)-1- | |
| | | | ((1r,4S)-4-methylcyclo- | |
| | | | hexyl)-2-oxoethyl)-4- | |
| | | | methyl-1,2,5-oxadiazole- | |
| | | | 3-carboxamide | |
| 63 | | | N-((S)-2-((5-fluoro-4-(((S)- | 20 mg, |
| | | | 2-oxo-4-(trifluoromethyl)- | 59% |
| | | | imidazolidin-1-yl)methyl)- | |
| | | | pyridin-2-yl)amino)-1- | |
| | | | ((1r,4S)-4-methylcyclo- | |
| | | | hexyl)-2-oxoethyl)-1- | |
| | | | isopropyl-1H-pyrazole-5- | |
| | | | carboxamide | |
| 64 | | | N-((S)-2-((5-fluoro-4-(((S)- | 21 mg, |
| | | | 2-oxo-4-(trifluoromethyl)- | 64% |
| | | | imidazolidin-1-yl)methyl)- | |
| | | | pyridin-2-yl)amino)-1- | |
| | | | ((1r,4S)-4-methylcyclo- | |
| | | | hexyl)-2-oxoethyl)-1- | |
| | | | methyl-1H-pyrazole-5- | |
| | | | carboxamide | |

### Example 61

LC/MS: m/z=568.3 [M+H]⁺; rt: 2.48 min (LC/MS-method A). ¹H NMR (400 MHz, DMSO-d₆) δ ppm: 10.80 (s, 1H), 9.16 (d, J=8.2 Hz, 1H), 8.31 (d, J=0.7 Hz, 1H), 8.02 (d, J=5.6 Hz, 1H), 7.69 (d, J=1.8, 1H), 4.61 (t, J=7.8 Hz, 1H), 4.48 (d, J=16.3 Hz, 1H), 4.36-4.45 (m, 1H), 4.29 (d, J=16.3 Hz, 1H), 3.63 (t, J=9.9 Hz, 1H), 3.36-3.38 (m, 1H), 2.22-2.30 (m, 1H), 1.74-1.85 (m, 2H), 1.73-1.64 (m, 2H), 1.55-1.62 (m, 1H), 1.03-1.33 (m, 5H), 0.95-1.00 (m, 2H), 0.80-0.93 (m, 5H).

### Example 62

LC/MS: m/z=542.2 [M+H]⁺; rt: 2.32 min (LC/MS-method A). ¹H NMR (400 MHz, DMSO-d₆) δ ppm: 10.78 (s, 1H), 9.08 (d, J=8.2 Hz, 1H), 8.31 (d, J=0.7 Hz, 1H), 8.02 (d, J=5.8 Hz, 1H), 7.68 (d, J=1.7 Hz, 1H), 4.59 (t, J=7.9 Hz, 1H), 4.48 (d, J=16.3 Hz, 1H), 4.37-4.45 (m, 1H), 4.28 (d, J=16.3 Hz, 1H), 3.63 (t, J=9.9 Hz, 1H), 3.33-3.38 (m, 1H), 2.46 (s, 3H), 1.74-1.85 (m, 2H), 1.73-1.63 (m, 2H), 1.54-1.62 (m, 1H), 1.16-1.34 (m, 2H), 1.01-1.14 (m, 1H), 0.80-0.92 (m, 5H).

### Example 63

LC/MS: m/z=568.3 [M+H]⁺; rt: 2.30 min (LC/MS-method A). ¹H NMR (400 MHz, DMSO-d₆) δ ppm: 10.67 (s, 1H), 8.42 (d, J=8.0 Hz, 1H), 8.30 (d, J=0.9 Hz, 1H), 8.02 (d, J=5.7 Hz, 1H), 7.67 (d, J=2.0 Hz, 1H), 7.49 (d, J=2.0 Hz, 1H), 6.93 (d, J=2.0 Hz, 1H), 5.37 (sep, J=6.6 Hz, 1H), 4.35-4.52 (m, 3H), 4.28 (d, J=16.3 Hz, 1H), 3.62 (t, J=10.0 Hz, 1H), 3.33-3.37 (m, 1H), 1.63-1.86 (m, 4H), 1.52-1.60 (m, 1H), 1.16-1.39 (m, 8H), 1.00-1.24 (m, 1H), 0.79-0.93 (m, 5H).

### Example 64

LC/MS: m/z=540.3 [M+H]⁺; rt: 2.08 min (LC/MS-method A). ¹H NMR (400 MHz, DMSO-d₆) δ ppm: 10.69 (s, 1H), 8.43 (d, J=8.1 Hz, 1H), 8.30 (d, J=0.9 Hz, 1H), 8.03 (d, J=5.8 Hz, 1H), 7.67 (d, J=1.9 Hz, 1H), 7.46 (d, J=2.0 Hz, 1H), 7.04 (d, J=2.0 Hz, 1H), 4.35-4.54 (m, 3H), 4.28 (d, J=16.3 Hz, 1H), 4.01 (s, 3H), 3.62 (t, J=10.0 Hz, 1H), 3.33-3.38 (m, 1H), 1.63-1.87 (m, 4H), 1.52-1.60 (m, 1H), 1.16-1.34 (m, 2H), 1.00-1.12 (m, 1H), 0.79-0.93 (m, 5H).

### Example 65 and Example 66: Tert-butyl ((S)-2-((4-((S or R)-2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)carbamate (DS1) and tert-butyl ((S)-2-((4-((R or S)-2-methoxy-1-((S)-2-oxo-4-(trifluoromethyl)imidazolidin-1-yl)ethyl)pyridin-2-yl)amino)-1-((r,4S)-4-methylcyclohexyl)-2-oxoethyl)carbamate (DS2)

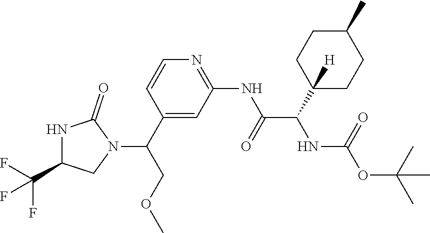

A solution of tert-butyl ((1S)-2-((4-(1-(((S)-2-amino-3,3,3-trifluoropropyl)amino)-2-methoxyethyl)pyridin-2-yl)amino)-1-((1r,4S)-4-methylcyclohexyl)-2-oxoethyl)carbamate (Intermediate 17; 3.61 g) in THE (48 ml) was heated to 60° C. CDI (2.75 g) was portion wise added to the mixture, and the reaction mixture was stirred at 60° C. for 1 h. Sodium hydroxide solution (5 N, 1.6 ml) was added and the mixture was stirred for 5 min, diluted with H₂O (16 ml) and extracted with EA (32 ml×2). The organic phase was washed with brine (24 ml×3), dried over anhydrous Na₂SO₄, filtered, and concentrated in vacuo. The residue was purified by column chromatography (Silica gel, PE/EA=1/1) to yield 3.13 g of a diastereomeric mixture of the title compounds, which was separated by SFC (DAICEL CHIRALPAK AD (250 mm*50 mm, 10 μm); B: [0.1% NH₃/H₂O in IPA]; B %: 40%-4 0%, 3.9 min; 200 ml/min) to yield 1.29 g of diastereomer 1 (DS1) and 1.15 g of diastereomer 2 (DS2).

### DS1:

Analytical SC data (Chiralpak AD-3; 50 mm×4.6 mm; 3 μm; B: PA (0.05% IDEA)]; B %: 5%-40%, flow 3 ml/min, 35° C.): rt: 1.54 min; >99.9% e.e. LC/MS: m/z=558.3 [M+H]⁺; rt: 2.43 min (LC/MS-method A). ¹H NMR (400 MHz, DMSO-d₆) 1 ppm 10.37 (s, 1H), 8.28 (d, J=5.2 Hz, 1H), 8.04 (s, 1H), 7.59 (d, J=2.0 Hz, 1H), 7.06 (dd, J=5.2 Hz, 0.9 Hz, 1H), 6.92 (d, J=8.8 Hz, 1H), 4.96-5.01 (m, 1H), 4.35-4.45 (m, 1H), 4.07 (br t, 1H), 3.72-3.85 (m, 2H), 3.46-3.55 (m, 2H), 3.29 (s, 3H), 1.47-1-74 (m, 5H), 0.97-1.42 (m, 12H), 0.75-0.89 (m, 5H).

### DS2:

Analytical SFC data (Chiralpak AD-3; 50 mm×4.6 mm; 3 μm; B: IPA (0.05% IDEA)]; B %: 5%-40%, flow 3 ml/min, 35° C.): rt: 1.87 min; >99.2% e.e. LC/MS: m/z=558.3 [M+H]⁺; rt: 2.37 min (LC/MS-method A). ¹H NMR (400 MHz, DMSO-d) ppm 10.34 (s, 1H), 8.28 (d, J=5.1 Hz, 1H), 8.00 (s, 1H), 7.62 (d, J=2.0 Hz, 1H), 6.98 (dd, J=5.2 Hz, 0.9 Hz, 1H), 6.90 (d, J=8.4 Hz, 1H), 4.97-5.04 (m, 1H), 4.38-4.49 (m, 1H), 4.02-4.10 (m, 1H), 3.79-3.86 (m, 1H), 3.71-3.79 (m, 2H), 3.31 (s, 3H), 3.26-3.29 (m, 1H), 1.46-1-76 (m, 5H), 0.96-1.44 (m, 12H), 0.75-0.89 (m, 5H).

Analogously to Example 2 were prepared:

| Ex. | Acid | Structure | Name | Yield |
|---|---|---|---|---|
| 67 | | | 1-fluoro-N-((S)-1-((1r,4S)- | 12 mg, |
| | | | 4-methylcyclohexyl)-2- | 53% |
| | | | oxo-2-((4-(((S)-2-oxo-4- | |
| | | | (trifluoromethyl)imidazolidin- | |
| | | | 1-yl)methyl)pyridin-2- | |
| | | | yl)amino)ethyl)cyclopropane- | |
| | | | 1-carboxamide | |
| 68 | stereomer 1 | | N-((S)-1-((1r,4S)-4- | 16 mg, |
| | | | methylcyclohexyl)-2-oxo- | 45% |
| | | | 2-((4-(((S)-2-oxo-4- | |
| | | | (trifluoromethyl)imidazolidin- | |
| | | | 1-yl)methyl)pyridin-2- | |
| | | | yl)amino)ethyl)-1-(4,4,4- | |
| | | | trifluoro-3-hydroxybutyl)- | |
| | | | 1H-pyrazole-5-carbox- | |
| | | | amide (stereomer 1) | |
| 69 | stereomer 2 | | N-((S)-1-((1r,4S)-4- | 18 mg, |
| | | | methylcyclohexyl)-2-oxo- | 50% |
| | | | 2-((4-(((S)-2-oxo-4- | |
| | | | (trifluoromethyl)imidazolidin- | |
| | | | 1-yl)methyl)pyridin-2- | |
| | | | yl)amino)ethyl)-1-(4,4,4- | |
| | | | trifluoro-3-hydroxybutyl)- | |
| | | | 1H-pyrazole-5-carbox- | |
| | | | amide (stereomer 2) | |

### Example 67

LC/MS: m/z=500.2 [M+H]⁺; rt: 2.11 min (LC/MS-method A). ¹H NMR (400 MHz, DMSO-de) δ ppm: 10.59 (s, 1H), 8.28 (d, J=5.0 Hz, 1H), 8.13 (br d, J=8.5 Hz, 1H), 7.97 (s, 1H), 7.65 (d, J=1.8 Hz, 1H), 6.94 (dd, J=5.1 Hz, 1.1 Hz, 1H), 4.35-4.47 (m, 3H), 4.19-4.25 (d, J=17.2 Hz, 1H), 3.59 (t, J=9.9 Hz, 1H), 3.28-3.30 (m, 1H), 1.50-1.83 (m, 5H), 1.10-1.39 (m 6H), 0.78-1.05 (m 6H).

### Example 68

LC/MS: m/z=634.4 [M+H]⁺; rt: 2.23 min (LC/MS-method A). ¹H NMR (400 MHz, DMSO-de) δ ppm: 10.61 (s, 1H), 8.48 (d, J=8.1 Hz, 1H), 8.28 (d, J=5.1 Hz, 1H), 7.98 (s, 1H), 7.64 (d, J=1.9 Hz, 1H), 7.52 (d, J=2.0 Hz, 1H), 7.04 (d, J=2.0 Hz, 1H), 6.94 (dd, J=5.1 Hz, 1.0 Hz, 1H), 6.31 (br s, 1H), 4.59-4.68 (m, 1H), 4.47-4.56 (m, 2H), 4.35-4.45 (m, 2H), 4.20 (d, J=16.2 Hz, 1H), 3.86-3.96 (m, 1H), 3.58 (t, J=10.0 Hz, 1H), 3.27-3.31 (m, 1H), 2.02-2.11 (m, 1H), 1.53-1.93 (m, 6H), 1.15-1.36 (m, 2H), 1.00-1.23 (m, 1H), 0.79-0.93 (m, 5H).

### Example 69

LC/MS: m/z=634.4 [M+H]⁺; rt: 2.21 min (LC/MS-method A). ¹H NMR (400 MHz, DMSO-de) δ ppm: 10.62 (s, 1H), 8.48 (d, J=8.1 Hz, 1H), 8.28 (d, J=5.1 Hz, 1H), 7.98 (s, 1H), 7.64 (d, J=1.9 Hz, 1H), 7.52 (d, J=2.0 Hz, 1H), 7.04 (d, J=2.0 Hz, 1H), 6.94 (dd, J=5.1 Hz, 1.0 Hz, 1H), 6.30 (br s, 1H), 4.49-4.63 (m, 3H), 4.35-4.45 (m, 2H), 4.20 (d, J=16.2 Hz, 1H), 3.88-4.00 (m, 1H), 3.58 (t, J=10.0 Hz, 1H), 3.27-3.32 (m, 1H), 2.02-2.11 (m, 1H), 1.53-1.93 (m, 6H), 1.15-1.35 (m, 2H), 1.00-1.23 (m, 1H), 0.79-0.94 (m, 5H).

### Example 70: (4-Methyl-1,2,5-oxadiazol-3-yl)methyl ((S)-1-(4,4-difluorocyclohexyl)-2-oxo-2-((4-(((S)-2-oxo-4-(trifluoromethyl)imidazolidin-1-yl)methyl)pyridin-2-yl)amino)ethyl)carbamate

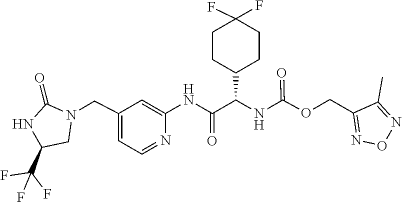 [SMILES: Cc1nonc1COC(=O)N[C@H](C(=O)Nc1cc(CN2C[C@@H](C(F)(F)F)NC2=O)ccn1)C1CCC(F)(F)CC1]

Following the procedure of Example 60, and using (S)-2-(4,4-difluorocyclohexyl)-2-((((4-methyl-1,2,5-oxadiazol-3-yl)methoxy)carbonyl)amino)acetic acid (Intermediate 22; 33 mg), 10 mg of the title compound was obtained. LC/MS: m/z=576.2 [M+H]⁺; rt: 1.89 min (LC/MS-method A). ¹H NMR (400 MHz, DMSO-de) 8 ppm 10.60 (d, J=3.1 Hz, 1H), 8.28 (d, J=5.1 Hz, 1H), 7.97 (br d, J=3.2 Hz, 1H), 7.87 (dd, J=8.3 Hz, 2.3 Hz, 1H), 7.66 (s, 1H), 6.93-6-96 (m, 1H), 5.26 (s, 2H), 4.18-4.48 (m, 4H), 3.55-3.63 (m, 1H), 2.39 (s, 3H), 1.26-2.11 (m, 10H).

### Example 71: Tert-butyl ((1S)-2-((4-(cyclopropyl(4,4,4-trifluorobutanamido)methyl)-pyridin-2-yl)amino)-1-((1r,4S)-4-methylcyclohexyl)-2-oxoethyl)carbamate Diastereomer 1

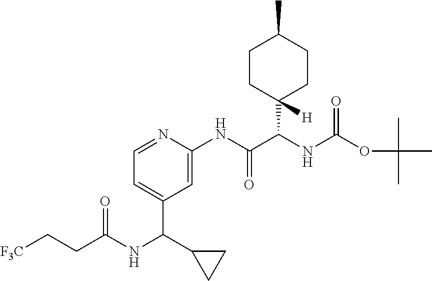 [SMILES: CC(C)(C)OC(=O)N[C@H](C(=O)Nc1cc(C(NC(=O)CCC(F)(F)F)C2CC2)ccn1)[C@H]1CC[C@H](C)CC1]

The title compound was prepared using the procedure described for tert-butyl ((2S)-1,1-dicyclopropyl-3-((4-(cyclopropyl(4,4,4-trifluorobutanamido)methyl)-pyridin-2-yl)amino)-3-oxopropan-2-yl)carbamate (Intermediate 34, step 6) employing (R)-2-((tert-butoxycarbonyl)amino)-2-((1r,4R)-4-methylcyclohexyl)acetic acid as the amino acid. The diastereomers were separated by chiral SFC (column: Daicel Chiralpak IA (250 mm×30 mm, 10 um); mobile phase: IPA:ACN (0.1% IPAm) to afford 140 mg of the title compound. LC/MS: m/z=541.3 [M+H]⁺; rt: 2.61 min (LC/MS-method A). ¹H NMR (400 MHz, DMSO-de) δ ppm 10.28 (s, 1H), 8.72 (d, J=7.7 Hz, 1H), 8.23 (d, J=5.1 Hz, 1H), 8.09 (s, 1H), 7.07 (dd, J=5.1 Hz, 1.0 Hz, 1H), 6.91 (br d, J=8.5 Hz, 1H), 4.14 (t, J=8.4 Hz, 1H), 4.05 (br t, J=7.8 Hz, 1H), 2.39-2.48 (m, 4H), 1.47-1.74 (m, 5H), 0.98-1.42 (m, 13H), 0.75-0.88 (m, 5H), 0.47-0.57 (m, 2H), 0.30-0.43 (m, 2H).

### Example 72: Tert-butyl ((1S)-2-((4-(cyclopropyl(4,4,4-trifluorobutanamido)-methyl)pyridin-2-yl)amino)-1-(4,4-difluorocyclohexyl)-2-oxoethyl)carbamate Diastereomer 1

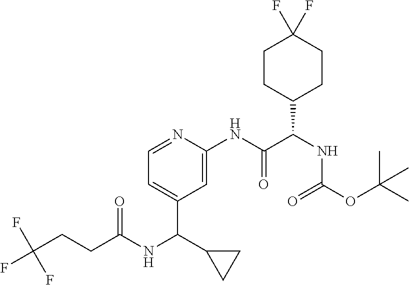 [SMILES: CC(C)(C)OC(=O)N[C@H](C(=O)Nc1cc(C(NC(=O)CCC(F)(F)F)C2CC2)ccn1)C1CCC(F)(F)CC1]

The title compound was prepared using the procedure described for tert-butyl ((S)-2-((4-((R)-cyclopropyl(4,4,4-trifluorobutanamido)methyl)pyridin-2-yl)amino)-1-((1r,4S)-4-methylcyclohexyl)-2-oxoethyl)carbamate (Example 71) using 2-((tert-butoxycarbonyl)amino)-2-(4,4-difluorocyclohexyl)acetic acid as the amino acid. The diastereomers were separated by chiral SFC (column: Regis (R,R) Whelk 01 (250 mm×30 mm, 10 um); mobile phase: Water+0.1% NH₃:MeOH and the first peak containing two diastereomers was collected and lyophilized to afford a white solid. The two diastereomers were purified by another chiral SFC purification (column: Daicel Chiralpak AD (250 mm×30 mm, 10 um) mobile phase: Water+0.1% NH₃: IPA to afford 551 mg of the title compound. LC/MS: m/z=563.3 [M+H]⁺; rt: 2.33 min (LC/MS-method A). ¹H NMR (400 MHz, DMSO-d₆) δ ppm 10.41 (s, 1H), 8.72 (br d, J=7.7 Hz, 1H), 8.24 (d, J=5.2 Hz, 1H), 8.10 (s, 1H), 7.04-7.10 (m, 2H), 4.11-4.24 (m, 2H), 2.39-2.49 (m, 4H), 1.95-2.08 (m, 2H), 1.57-1.85 (m, 5H), 1.23-1.45 (m, 11H), 1.03-1.12 (m, 1H), 0.48-0.56 (m, 2H), 0.30-0.43 (m, 2H).

### Example 73: Tert-butyl ((1S)-2-((4-(cyclopropyl(4,4,4-trifluorobutanamido)methyl)-pyridin-2-yl)amino)-1-((1r,4S)-4-methylcyclohexyl)-2-oxoethyl)carbamate Diastereomer 2

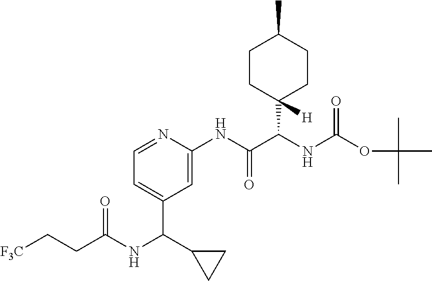 [SMILES: CC(C)(C)OC(=O)N[C@H](C(=O)Nc1cc(C(NC(=O)CCC(F)(F)F)C2CC2)ccn1)[C@H]1CC[C@H](C)CC1]

The title compound was obtained as the second diastereomer from Example 71. LC/MS: m/z=541.3 [M+H]⁺; rt: 2.61 min (LC/MS-method A). ¹H NMR (400 MHz, DMSO-de) δ ppm 10.26 (s, 1H), 8.71 (d, J=7.7 Hz, 1H), 8.23 (d, J=5.0 Hz, 1H), 8.09 (s, 1H), 7.07 (dd, J=5.1 Hz, 1.0 Hz, 1H), 6.92 (br d, J=8.5 Hz, 1H), 4.15 (t, J=8.4 Hz, 1H), 4.04 (br t, J=7.8 Hz, 1H), 2.39-2.48 (m, 4H), 1.47-1.74 (m, 5H), 0.98-1.42 (m, 13H), 0.75-0.88 (m, 5H), 0.47-0.57 (m, 2H), 0.30-0.43 (m, 2H).

### Example 74: Tert-butyl ((1S)-2-((4-(cyclopropyl(4,4,4-trifluorobutanamido)-methyl)pyridin-2-yl)amino)-1-(4,4-difluorocyclohexyl)-2-oxoethyl)carbamate Diastereomer 2

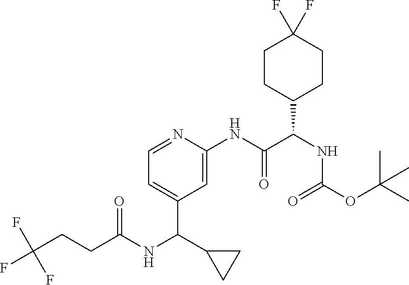 [SMILES: CC(C)(C)OC(=O)N[C@H](C(=O)Nc1cc(C(NC(=O)CCC(F)(F)F)C2CC2)ccn1)C1CCC(F)(F)CC1]

The title compound was obtained as the second diastereomer from Example 72. LC/MS: m/z=563.3 [M+H]⁺; rt: 2.34 min (LC/MS-method A). ¹H NMR (400 MHz, DMSO-de) δ ppm 10.39 (s, 1H), 8.71 (br d, J=7.7 Hz, 1H), 8.24 (d, J=5.2 Hz, 1H), 8.10 (s, 1H), 7.04-7.10 (m, 2H), 4.11-4.24 (m, 2H), 2.39-2.49 (m, 4H), 1.95-2.08 (m, 2H), 1.57-1.85 (m, 5H), 1.21-1.45 (m, 11H), 1.04-1.14 (m, 1H), 0.48-0.56 (m, 2H), 0.30-0.42 (m, 2H).

Using (2S)-2-amino-N-(4-((R or S)-2-methoxy-1-((S)-2-oxo-4-(trifluoromethyl)-imidazolidin-1-yl)ethyl)pyridin-2-yl)-2-((1r,4S)-4-methylcyclohexyl)acetamide hydrochloride salt (DS1; Intermediate 19) as starting material, 4 h reaction time, and standing overnight analogously to Example 2, were prepared:

| Ex. | Acid | Structure | Name | Yield |
|---|---|---|---|---|
| 75 | | | N-((1S)-2-((4-(2- | 12 mg, |
| | | | methoxy-1-((S)-2-oxo-4- | 45% |
| | | | (trifluoromethyl)imidazolidin- | |
| | | | 1-yl)ethyl)pyridin-2- | |
| | | | yl)amino)-1-((1r,4S)-4- | |
| | | | methylcyclohexyl)-2- | |
| | | | oxoethyl)-4-methyl-1,2,5- | |
| | | | oxadiazole-3-carbox- | |
| | | | amide (DS1) | |
| 76 | | | N-((1S)-2-((4-(2-methoxy- | 13 mg, |
| | | | 1-((S)-2-oxo-4-(trifluoro- | 48% |
| | | | methyl)imidazolidin-1- | |
| | | | yl)ethyl)pyridin-2- | |
| | | | yl)amino)-1-((1r,4S)-4- | |
| | | | methylcyclohexyl)-2- | |
| | | | oxoethyl)-1-methyl-1H- | |
| | | | pyrazole-5-carboxamide | |
| | | | (DS1) | |
| 77 | | | 3-isopropyl-N-((1S)-2-((4- | 9 mg, |
| | | | (2-methoxy-1-((S)-2-oxo- | 31% |
| | | | 4-(trifluoromethyl)- | |
| | | | imidazolidin-1- | |
| | | | yl)ethyl)pyridin-2- | |
| | | | yl)amino)-1-((1r,4S)-4- | |
| | | | methylcyclohexyl)-2- | |
| | | | oxoethyl)isoxazole-4- | |
| | | | carboxamide (DS1) | |
| 78 | | | 1-isopropyl-N-((1S)-2-((4- | 11 mg, |
| | | | (2-methoxy-1-((S)-2-oxo- | 39% |
| | | | 4-(trifluoromethyl)- | |
| | | | imidazolidin-1- | |
| | | | yl)ethyl)pyridin-2- | |
| | | | yl)amino)-1-((1r,4S)-4- | |
| | | | methylcyclohexyl)-2- | |
| | | | oxoethyl)-1H-pyrazole-5- | |
| | | | carboxamide (DS1) | |

### Example 75

LC/MS: m/z=568.3 [M+H]⁺; rt: 2.38 min (LC/MS-method A). ¹H NMR (400 MHz, DMSO-d₆) δ ppm: 10.73 (s, 1H), 9.08 (d, J=8.2 Hz, 1H), 8.31 (d, J=5.2 Hz, 1H), 8.05 (s, 1H), 7.59 (d, J=2.0 Hz, 1H), 7.08 (dd, J=5.2 Hz, 11.2 Hz, 1H), 4.98 (dd, J=7.9 Hz, 5.7 Hz, 1H), 4.60 (dd, J=8.2 Hz, 7.8 Hz, 1H), 4.40 (m, 1H), 4.01 (s, 3H), 3.82 (dd, J=10.4 Hz, 7.9 Hz, 1H), 3.75 (dd, J=10.4 Hz, 5.7 Hz, 1H), 3.54-3.47 (m, 2H), 3.29 (s, 3H), 2.47 (s, 3H), 1.84-1.58 (m, 5H), 1.31-1.19 (m, 2H), 11.08 (m, 1H), 0.92-0.83 (m, 2H), 0.85 (d, J=6.5 Hz, 3H).

### Example 76

LC/MS: m/z=566.3 [M+H]⁺; rt: 2.14 min (LC/MS-method A). ¹H NMR (400 MHz, DMSO-de) 6 ppm: 10.63 (s, 1H), 8.43 (d, J=8.1 Hz, 1H), 8.30 (d, J=5.2 Hz, 1H), 8.05 (s, 1H), 7.58 (d, J=2.0 Hz, 1H), 7.46 (d, J=2.0 Hz, 1H), 7.07 (dd, J=5.2 Hz, 1.2 Hz, 1H), 7.04 (d, J=2.0 Hz, 1H), 4.98 (dd, J=7.9 Hz, 5.7 Hz, 1H), 4.52 (dd, J=8.1 Hz, 8.0 Hz, 1H), 4.39 (m, 1H), 4.01 (s, 3H), 3.81 (dd, J=10.4 Hz, 7.9 Hz, 1H), 3.74 (dd, J=10.4 Hz, 5.7 Hz, 1H), 3.54-3.46 (m, 2H), 3.29 (s, 3H), 1.84-1.56 (m, 5H), 1.31-1.19 (m, 2H), 1.06 (m, 1H), 0.92-0.82 (m, 2H), 0.85 (d, J=6.5 Hz, 3H).

### Example 77

LC/MS: m/z=595.3 [M+H]⁺; rt: 2.39 min (LC/MS-method A). ¹H NMR (400 MHz, DMSO-d₆) δ ppm: 10.63 (s, 1H), 9.36 (s, 1H), 8.42 (d, J=8.0 Hz, 1H), 8.30 (d, J=5.2 Hz, 1H), 8.05 (s, 1H), 7.58 (d, J=2.0 Hz, 1H), 7.07 (dd, J=5.2 Hz, 1.2 Hz, 1H), 4.98 (dd, J=7.9 Hz, 5.7 Hz, 1H), 4.55 (dd, J=8.1 Hz, 8.0 Hz, 1H), 4.38 (m, 1H), 3.81 (dd, J=10.4 Hz, 7.9 Hz, 1H), 3.75 (dd, J=10.4 Hz, 5.7 Hz, 1H), 3.53-3.47 (m, 2H), 3.41 (m, 1H), 3.29 (s, 3H), 1.83-1.56 (m, 5H), 1.31-1.19 (m, 2H), 1.23 (d, J=6.9 Hz, 3H), 1.20 (d, J=6.9 Hz, 3H), 1.08 (m, 1H), 0.91-0.80 (m, 2H), 0.85 (d, J=6.5 Hz, 3H).

### Example 78

LC/MS: m/z=594.4 [M+H]⁺; rt: 2.35 min (LC/MS-method A). ¹H NMR (400 MHz, DMSO-de) 6 ppm: 10.61 (s, 1H), 8.42 (d, J=8.0 Hz, 1H), 8.30 (d, J=5.2 Hz, 1H), 8.05 (s, 1H), 7.58 (d, J=2.0 Hz, 1H), 7.50 (d, J=1.9 Hz, 1H), 7.07 (dd, J=5.2 Hz, 1.1 Hz, 1H), 6.94 (d, J=1.9 Hz, 1H), 5.37 (m, 1H), 4.98 (dd, J=7.9 Hz, 5.7 Hz, 1H), 4.51 (dd, J=8.3 Hz, 8.0 Hz, 1H), 4.39 (m, 1H), 3.81 (dd, J=10.4 Hz, 7.9 Hz, 1H), 3.75 (dd, J=10.4 Hz, 5.7 Hz, 1H), 3.54-3.46 (m, 2H), 3.29 (s, 3H), 1.83-1.56 (m, 5H), 1.36 (d, J=6.6 Hz, 3H), 1.34 (d, J=6.6 Hz, 3H), 1.31-1.19 (m, 2H), 1.07 (m, 1H), 0.91-0.82 (m, 2H), 0.85 (d, J=6.5 Hz, 3H).

### Example 79: Tert-butyl ((S)-2-((4-((3,5-dimethyl-1H-pyrazol-4-yl)methyl)pyridin-2-yl)amino)-1-((1r,4S)-4-methylcyclohexyl)-2-oxoethyl)carbamate

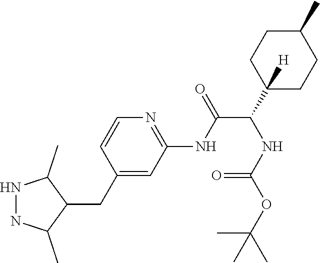

Dry THF (1.2 ml) and aqueous K₃PO₄ solution (3 M, 25 μl) were added to mixture of tert-butyl ((S)-2-((4-(bromomethyl)pyridin-2-yl)amino)-1-((1r,4S)-4-methylcyclohexyl)-2-oxoethyl)carbamate (Intermediate 23; 11 mg), (3,5-Dimethyl-1H-pyrazole-4-yl)boronic acid (3.5 mg), and 1,1′-bis(diphenylphosphino)ferrocene-palladium(II)dichloride DCM complex (2 mg) with stirring under Ar. After stirring for 3 h at 70° C. and standing overnight at RT water was added, and the aqueous mixture was extracted with EA (3×). The combined organic phases were dried over sodium sulphate, filtered, and concentrated in vacuo. The residue was purified by preparative RP HPLC (Purosphere® STAR-RP18, 25 mm×250 mm, 10 μm; 30 ml/min, from 95% H₂O/5% ACN to 10% H₂O/90% ACN in 45 min). The pure product containing fractions were combined, the ACN was partly removed, and the residue was freeze dried. After a further purification by silica gel column chromatography (4 g silica gel; gradient: DCM/EtOH: 100% DCM for 5 min, 100% to 95% DCM in 40 min), combining the product containing fractions and concentrating them in vacuo the residue was freeze dried from water/ACN to yield 1.2 mg of the title compound. LC/MS: m/z=456.3 [M+H]⁺; rt: 2.17 min (LC/MS-method A). ¹H NMR (400 MHz, DMSO-de) δ ppm: 10.22 (s, 1H), 8.15 (d, J=5.1 Hz, 1H), 7.91 (s, 1H), 6.89 (d, J=8.4 Hz, 1H), 6.84 (dd, J=5.1 Hz, 1.3 Hz, 1H), 4.04 (dd, J=8.4 Hz, 7.0 Hz, 1H), 3.66 (s, 2H), 2.06 (s, 6H), 1.69-1.45 (m, 5H), 1.37 (s, 9H), 1.28-1.12 (m, 2H), 1.02 (m, 1H), 0.86-0.78 (m, 2H), 0.83 (d, J=6.5 Hz, 3H).

### Example 80: tert-butyl ((S)-1-((1r,4S)-4-methylcyclohexyl)-2-oxo-2-((5-(((S)-2-oxo-4-(trifluoromethyl)imidazolidin-1-yl)methyl)pyridin-3-yl)amino)ethyl)carbamate and tert-butyl ((R)-1-((1r,4R)-4-methylcyclohexyl)-2-oxo-2-((5-(((S)-2-oxo-4-(trifluoromethyl)imidazolidin-1-yl)methyl)pyridin-3-yl)amino)ethyl)carbamate

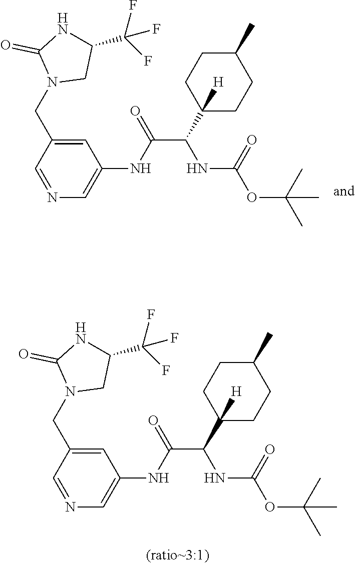

(2S)-2-(Tert-butoxycarbonylamino)-2-(4-methylcyclohexyl)acetic acid (90 mg) was dissolved in DCM (4 ml) and DMAP (77 mg) and EDC (121 mg) were added with stirring. After 5 min, (S)-1-((5-aminopyridin-3-yl)methyl)-4-(trifluoromethyl)imidazolidin-2-one (Intermediate 24, 82 mg) was added. After 1 h, the mixture was poured onto saturated NaHCO₃-solution. The aqueous phase was extracted with DCM (2×). The combined organic phases were dried with magnesium sulphate, filtered, and concentrated in vacuo. The residue was purified by preparative HPLC (Agilent Prep C18, 30 mm×250 mm, 10 μm; 50 ml/min, from 90% H₂O/10% ACN to 10% H₂O/90% ACN in 12.5 min). The product containing fractions were combined, the ACN was partly removed, and the residue freeze dried to yield 104 mg of the title compounds in a ratio of about 3:1 determined by ¹H NMR. LC/MS: m/z=514.3 [M+H]⁺; rt: 2.12 min (LC/MS-method A). ¹H NMR (400 MHz, DMSO-de) δ ppm: 10.23 (s, 1H), 8.71/8.68 (d, J=2.0, 1H), 8.15 (d, J=1.8 Hz, 1H), 7.93/7.91 (dd, J=2.0 Hz, 1.8 Hz, 1H), 7.61 (d, J=2.0, 1H), 6.91 (d, J=8.2 Hz, 1H), 4.42-4.32 (m, 2H), 4.24 (d, J=15.5 Hz, 1H), 3.93 (dd, J=8.2 Hz, 7.6 Hz, 1H), 3.56 (m, 1H), 3.27 (m, 1H), 1.73-1.49 (m, 5H), 1.38 (s, 9H), 1.32-0.94 (m, 3H), 0.88-0.78 (m, 2H), 0.83 (d, J=6.5 Hz, 3H) [bold values correspond to signals of the main diasteromer].

### Example 81: Benzyl ((rac)-3-(tert-butoxy)-1-oxo-1-((4-(((S)-2-oxo-4-(trifluoromethyl)-imidazolidin-1-VI)methyl)pyridin-2-yl)amino)propan-2-yl)carbamate

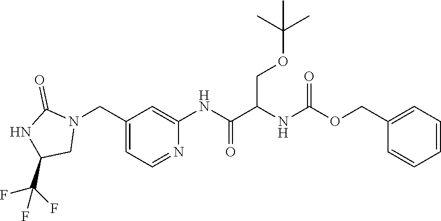 [SMILES: CC(C)(C)OCC(NC(=O)OCc1ccccc1)C(=O)Nc1cc(CN2C[C@@H](C(F)(F)F)NC2=O)ccn1]

(2S)-2-(Benzyloxycarbonylamino)-3-tert-butoxy-propanoic acid (60 mg) was dissolved in DCM (3 ml), and DMAP (50 mg) and EDC (78 mg) were added with stirring. After 5 min, (S)-1-((2-aminopyridin-4-yl)methyl)-4-(trifluoromethyl)imidazolidin-2-one (Intermediate 15, 53 mg) was added. After 1 h, the mixture was poured onto saturated NaHCO₃-solution. The aqueous phase was extracted with DCM (2×). The combined organic phases were dried with magnesium sulphate, filtered, and concentrated in vacuo. The residue was purified by silica gel chromatography (silica gel 12 g, DCM/EtOH: 100:0 for 5 min, 100:0 to 92:8 in 45 min, 92:8 for 5 min). The pure compound containing fractions were combined and concentrated in vacuo. The residue was dissolved in ACN/water and freeze dried to yield 32 mg of the title compound. LC/MS: m/z=538.3 [M+H]⁺; rt: 2.18 min (LC/MS-method A).

Using rac-2-amino-3-(tert-butoxy)-N-(4-(((S)-2-oxo-4-(trifluoromethyl)-imidazolidin-1-yl)methyl)pyridin-2-yl)propanamide (Intermediate 25) as starting material analogously to Example 2, were prepared:

| Ex. | Acid | Structure | Name | Yield |
|---|---|---|---|---|
| 82 | | | N-(3-(tert-butoxy)-1-oxo-1- | 5 mg, |
| | | | ((4-(((S)-2-oxo-4- | 36% |
| | | | (trifluoromethyl)imidazolidin- | |
| | | | 1-yl)methyl)pyridin-2- | |
| | | | yl)amino)propan-2-yl)-4- | |
| | | | cyclopropyl-1,2,5- | |
| | | | oxadiazole-3-carboxamide | |
| 83 | | | N-(3-(tert-butoxy)-1-oxo-1- | 5 mg, |
| | | | ((4-(((S)-2-oxo-4- | 37% |
| | | | (trifluoromethyl)imidazolidin- | |
| | | | 1-yl)methyl)pyridin-2- | |
| | | | yl)amino)propan-2-yl)-1- | |
| | | | methyl-1H-pyrazole-5- | |
| | | | carboxamide | |

### Example 82

LC/MS: m/z=540.2 [M+H]⁺; rt: 2.21 min (LC/MS-method A). ¹H NMR (400 MHz, DMSO-de) δ ppm: 10.66/10.65 (s, 1H), 9.02/9.01 (d, J=7.8 Hz, 1H), 8.30 (d, J=5.2 Hz, 1H), 7.97 (s, 1H), 7.65 (d, J=2.0 Hz, 1H), ~6.96 (m, 1H), 4.85 (m, 1H), 4.41 (m, 1H), ~4.40 (d, J=16.2 Hz, 1H), ~4.23 (d, J=16.2 Hz, 1H), 3.73 (d, J=5.7 Hz, 2H), 3.59 (dd, J=10.0 Hz, 10.0 Hz, 1H) ~3.30 (dd, 1H, signal below water signal), 2.33 (m, 1H), 1.14 (m, 2H), 1.13 (s, 9H), 1.00 (m, 2H).

### Example 83

LC/MS: m/z=512.2 [M+H]⁺; rt: 1.75 min (LC/MS-method A). ¹H NMR (400 MHz, DMSO-de) δ ppm: 10.51/10.50 (s, 1H), 8.45 (d, J=8.0 Hz, 1H), 8.29 (d, J=5.2 Hz, 1H), 7.97 (s, 1H), 7.64 (d, J=2.0 Hz, 1H), 7.48 (d, J=2.0 Hz, 1H), 6.984/6.976 (d, J=2.0 Hz, 1H), ~6.95 (m, 1H), 4.78 (m, 1H), 4.60 (dd, J=8.1 Hz, 7.7 Hz, 1H), 4.44 (m, 1H), 4.39/4.37 (d, J=16.2 Hz, 1H), 4.23/4.21 (d, J=16.2 Hz, 1H), 4.03 (s, 3H), 3.72-3.64 (m, 2H), 3.58 (dd, J=10.0 Hz, 10.0 Hz, 1H) ~3.30 (dd, 1H, signal below water signal), 1.14 (s, 9H).

### Example 84: 4-Cyclopropyl-N—((S)-1-((1r,4S)-4-methylcyclohexyl)-2-oxo-2-((5-(((S)-2-oxo-4-(trifluoromethyl)imidazolidin-1-yl)methyl)pyridin-3-yl)amino)ethyl)-1,2,5-oxadiazole-3-carboxamide and 4-Cyclopropyl-N—((R)-1-((1r,4R)-4-methylcyclo-hexyl)-2-oxo-2-((5-(((S)-2-oxo-4-(trifluoromethyl)imidazolidin-1-yl)methyl)pyridin-3-yl)amino)ethyl)-1,2,5-oxadiazole-3-carboxamide

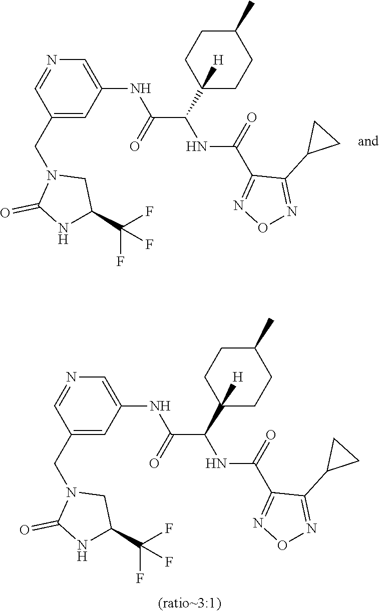

Analogously to Example 2, a 3:1 mixture of (S)-2-amino-2-((1r,4S)-4-methylcyclohexyl)-N-(5-(((S)-2-oxo-4-(trifluoromethyl)imidazolidin-1-yl)methyl)pyridin-3-yl)acetamide and (R)-2-amino-2-((1r,4R)-4-methylcyclohexyl)-N-(5-(((S)-2-oxo-4-(trifluoromethyl)imidazolidin-1-yl)methyl)pyridin-3-yl)acetamide (Intermediate 26; 20 mg) gave 13 mg of the title compounds in a ratio of about 3:1 determined by ¹H NMR. LC/MS: m/z=550.3 [M+H]⁺; rt: 2.25 min (LC/MS-method A). ¹H NMR (400 MHz, DMSO-d₆) δ ppm: 10.50 (s, 1H), 9.25 (d, J=8.0 Hz, 1H), 8.73/8.70 (d, J=2.0, 1H), 8.18 (d, J=1.7 Hz, 1H), 7.95/7.92 (dd, J=2.0 Hz, 1.7 Hz, 1H), 7.61 (d, J=2.0, 1H), 4.47 (dd, J=8.0 Hz, 8.0 Hz, 1H), 4.39 (m, 1H), 4.36 (d, J=15.6, 1H), 4.25 (m, 1H), 3.56 (m, 1H), 3.28 (m, 1H), 2.26 (m, 1H), 1.84-1.59 (m, 5H), 1.34-0.82 (m, 12H), 0.85 (d, J=6.5 Hz, 3H) [bold values correspond to signals of the main diastereomer].

### Example 85: N-((1S)-2-((4-(2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)-1-methyl-1H-pyrazole-5-carboxamide (DS2)

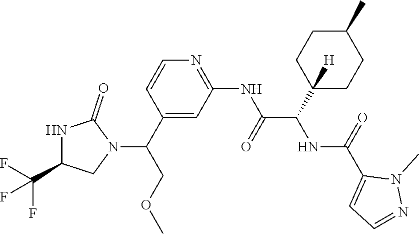

To a mixture of (2S)-2-amino-N-(4-((R or S)-2-methoxy-1-((S)-2-oxo-4-(trifluoromethyl)imidazolidin-1-yl)ethyl)pyridin-2-yl)-2-((1r,4S)-4-methylcyclohexyl)-acetamide hydrochloride salt (Intermediate 18 DS2, 25 mg), 2-methylpyrazole-3-carboxylic acid (7 mg), and DCM (3.5 ml) DIPEA (33 μl) and T3P (98 μl) were added. After 3.5 h additional DIPEA (40 μl) and T3P (99 μl) were added to complete the reaction (reaction monitored by LC/MS), and stirring was continued for 3 h. After standing overnight additional DIPEA (40 μl) and T3P (99 μl) were added and the mixture was heated for 6 h at 40° C. After cooling saturated Na₂CO₃ solution was added, and the aqueous phase was extracted with EA (3×). The combined organic phases were dried with sodium sulphate, filtered, and concentrated in vacuo. The residue was purified by preparative RP HPLC (Purosphere® STAR-RP18, 25 mm×250 mm, 10 μm; 30 ml/min, from 95% H₂O/5% ACN to 10% H₂O/90% ACN in 45 min). The pure product containing fractions were combined, the ACN was partly removed and the residue freeze dried to yield 7 mg of the title compound. LC/MS: m/z=566.3 [M+H]⁺; rt: 2.03 min (LC/MS-method A). ¹H NMR (400 MHz, DMSO-de) δ ppm: 10.60 (s, 1H), 8.43 (d, J=8.0 Hz, 1H), 8.29 (d, J=5.2 Hz, 1H), 8.01 (s, 1H), 7.62 (d, J=2.0, 1H), 7.46 (d, J=2.0 Hz, 1H), 7.03 (d, J=2.0 Hz, 1H), 6.99 (dd, J=5.2 Hz, 1.1 Hz, 1H), 5.00 (dd, J=8.1 Hz, 5.1 Hz, 1H), 4.51 (dd, J=8.6 Hz, 8.0 Hz, 1H), 4.44 (m, 1H), 4.01 (s, 3H), 3.83 (dd, J=10.5 Hz, 8.3 Hz, 1H), 3.76-3.71 (m, 2H), 3.30 (s, 3H), 3.28 (m, 1H), 1.85-1.56 (m, 5H), 1.31-1.19 (m, 2H), 1.06 (m, 1H), 0.92-0.81 (m, 2H), 0.85 (d, J=6.5 Hz, 3H).

Using (2S)-2-amino-N-(4-((R or S)-2-methoxy-1-((S)-2-oxo-4-(trifluoromethyl)-imidazolidin-1-yl)ethyl)pyridin-2-yl)-2-((1r,4S)-4-methylcyclohexyl)-acetamide hydrochloride salt (Intermediate 18, DS2) as starting material analogously to Example 92, were prepared:

| Ex. | Acid | Structure | Name | Yield |
|---|---|---|---|---|
| 86 | 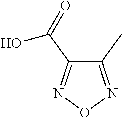 | 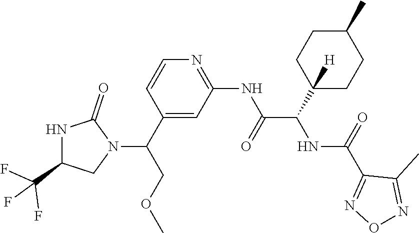 | N-((1S)-2-((4-(2-methoxy- 1-((S)-2-oxo-4-(trifluoro- methyl)imidazolidin-1- yl)ethyl)pyridin-2-yl)amino)- 1-((1r,4S)-4-methylcyclo- hexyl)-2-oxoethyl)-4- methyl-1,2,5-oxadiazole-3- carboxamide (DS2) | 3 mg, 11% |
| 87 | 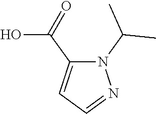 | 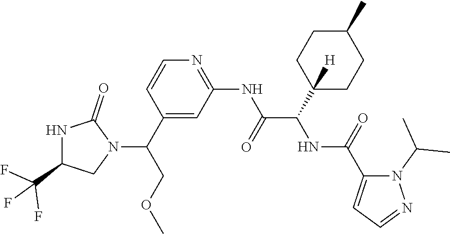 | 1-isopropyl-N-((1S)-2-((4- (2-methoxy-1-((S)-2-oxo-4- (trifluoromethyl)imidazolidin- 1-yl)ethyl)pyridin-2- yl)amino)-1-((1r,4S)-4- methylcyclohexyl)-2- oxoethyl)-1H-pyrazole-5- carboxamide (DS2) | 13 mg, 46% |
| 88 | 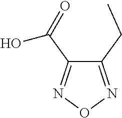 | 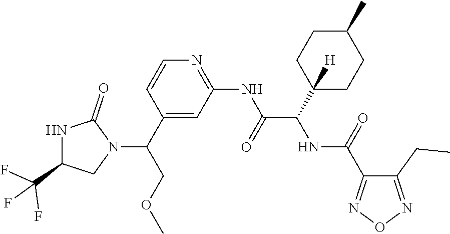 | 4-ethyl-N-((1S)-2-((4-(2- methoxy-1-((S)-2-oxo-4- (trifluoromethyl)imidazolidin- 1-yl)ethyl)pyridin-2- yl)amino)-1-((1r,4S)-4- methylcyclohexyl)-2- oxoethyl)-1,2,5-oxadiazole- 3-carboxamide (DS2) | 15 mg, 56% |
| 89 | 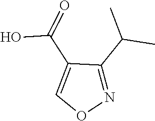 | 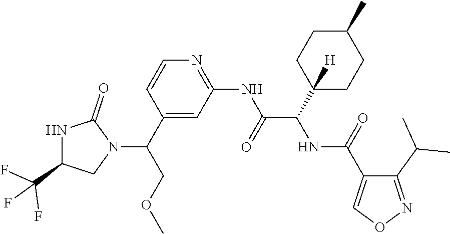 | 3-isopropyl-N-((1S)-2-((4- (2-methoxy-1-((S)-2-oxo-4- (trifluoromethyl)imidazolidin- 1-yl)ethyl)pyridin-2- yl)amino)-1-((1r,4S)-4- methylcyclohexyl)-2- oxoethyl)isoxazole-4- carboxamide (DS2) | 16 mg, 55% |
| 90 | 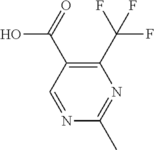 | 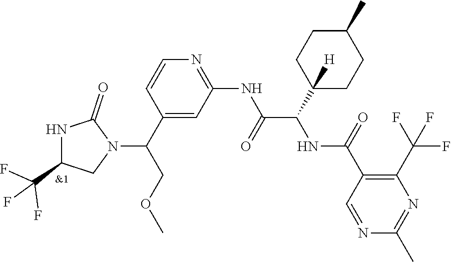 | N-((1S)-2-((4-(2-methoxy-1- ((RS)-2-oxo-4- (trifluoromethyl)imidazolidin- 1-yl)ethyl)pyridin-2- yl)amino)-1-((1r,4S)-4- methylcyclohexyl)-2- oxoethyl)-2-methyl-4- (trifluoromethyl)pyrimidine- 5-carboxamide (DS2) | 7 mg, 24% |
| 91 | 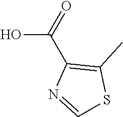 | 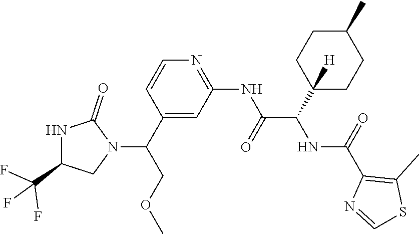 | N-((1S)-2-((4-(2-methoxy-1- ((S)-2-oxo-4- (trifluoromethyl)imidazolidin- 1-yl)ethyl)pyridin-2- yl)amino)-1-((1r,4S)-4- methylcyclohexyl)-2- oxoethyl)-5-methylthiazole- 4-carboxamide (DS2) | 12 mg, 45% |
| 92 | | | 5-cyano-N-((1S)-2-((4-(2- methoxy-1-((S)-2-oxo-4- (trifluoromethyl)imidazolidin- 1-yl)ethyl)pyridin-2- yl)amino)-1-((1r,4S)-4- methylcyclohexyl)-2- oxoethyl)thiazole-4- carboxamide (DS2) | 18 mg, 63% |
| 93 | | | N-((1S)-2-((4-(2-methoxy-1- ((S)-2-oxo-4-(trifluoro- methyl)imidazolidin-1- yl)ethyl)pyridin-2-yl)amino)- 1-((1r,4S)-4-methylcyclo- hexyl)-2-oxoethyl)-1- methyl-1H-tetrazole-5- carboxamide (DS2) | 19 mg, 58% |
| 94 | | | 6-bromo-N-((1S)-2-((4-(2- methoxy-1-((S)-2-oxo-4- (trifluoromethyl)imidazolidin- 1-yl)ethyl)pyridin-2- yl)amino)-1-((1r,4S)-4- methylcyclohexyl)-2- oxoethyl)-3-methyl- picolinamide (DS2) | 25 mg, 64% |
| 95 | | | 2-chloro-N-((1S)-2-((4-(2- methoxy-1-((S)-2-oxo-4- (trifluoromethyl)imidazolidin- 1-yl)ethyl)pyridin-2-yl)- amino)-1-((1r,4S)-4- methylcyclohexyl)-2-oxo- ethyl)-5-methyl-pyrimidine- 4-carboxamide (DS2) | 36 mg, 60% |

### Example 86

LC/MS: m/z=568.3 [M+H]⁺; rt: 2.29 min (LC/MS-method A). ¹H NMR (400 MHz, DMSO-d₆) δ ppm: 10.70 (s, 1H), 9.08 (d, J=8.1 Hz, 1H), 8.30 (d, J=5.2 Hz, 1H), 8.01 (s, 1H), 7.63 (d, J=2.0 Hz, 1H), 7.01 (dd, J=5.2 Hz, 1.1 Hz, 1H), 5.01 (dd, J=8.0 Hz, 5.1 Hz, 1H), 4.60 (dd, J=8.1 Hz, 7.7 Hz, 1H), 4.44 (m, 1H), 3.83 (dd, J=10.5 Hz, 8.3 Hz, 1H), 3.77-3.72 (m, 2H), ~3.30 (s, 3H, signal below water signal), 3.29 (dd, 1H, signal below water signal), 2.47 (s, 3H), 1.82-1.58 (m, 5H), 1.30-1.19 (m, 2H), 1.08 (m, 1H), 0.91-0.82 (m, 2H), 0.85 (d, J=6.5 Hz, 3H).

### Example 87

LC/MS: m/z=594.3 [M+H]⁺; rt: 2.25 min (LC/MS-method A). ¹H NMR (400 MHz, DMSO-d₆) δ ppm: 10.58 (s, 1H), 8.42 (d, J=8.0 Hz, 1H), 8.29 (d, J=5.2 Hz, 1H), 8.01 (s, 1H), 7.62 (d, J=2.0 Hz, 1H), 7.49 (d, J=2.0 Hz, 1H), 6.99 (dd, J=5.2 Hz, 1.1 Hz, 1H), 6.92 (d, J=2.0 Hz, 1H), 5.37 (m, 1H), 5.01 (dd, J=8.1 Hz, 5.1 Hz, 1H), 4.49 (dd, J=8.5 Hz, 8.0 Hz, 1H), 4.44 (m, 1H), 3.83 (dd, J=10.5 Hz, 8.3 Hz, 1H), 3.76-3.72 (m, 2H), 3.41 (m, 1H), 3.30 (s, 3H), 3.28 (dd, 1H, signal below water signal), 1.84-1.56 (m, 5H), 1.35 (d, J=6.6 Hz, 3H), 1.33 (d, J=6.6 Hz, 3H), 1.31-1.18 (m, 2H), 1.07 (m, 1H), 0.91-0.81 (m, 2H), 0.85 (d, J=6.5 Hz, 3H).

### Example 88

LC/MS: m/z=582.3 [M+H]⁺; rt: 2.42 min (LC/MS-method A). ¹H NMR (400 MHz, DMSO-de) δ ppm: 10.70 (s, 1H), 9.11 (d, J=8.1 Hz, 1H), 8.30 (d, J=5.2 Hz, 1H), 8.01 (s, 1H), 7.63 (d, J=2.0 Hz, 1H), 7.01 (dd, J=5.2 Hz, 1.1 Hz, 1H), 5.01 (dd, J=8.0 Hz, 5.1 Hz, 1H), 4.60 (dd, J=8.1 Hz, 7.7 Hz, 1H), 4.44 (m, 1H), 3.83 (dd, J=10.5 Hz, 8.3 Hz, 1H), 3.76-3.71 (m, 2H), 3.41 (m, 1H), 3.30 (s, 3H), 3.27 (m, 1H), 1.84-1.56 (m, 5H), ~1.31-1.20 (m, 2H), 1.23 (d, J=7.0 Hz, 3H), 1.20 (d, J=7.0 Hz, 3H), 1.08 (m, 1H), 0.91-0.81 (m, 2H), 0.85 (d, J=6.5 Hz, 3H).

### Example 89

LC/MS: m/z=595.3 [M+H]⁺; rt: 2.31 min (LC/MS-method A). ¹H NMR (400 MHz, DMSO-de) δ ppm: 10.59 (s, 1H), 9.35 (s, 1H), 8.41 (d, J=8.0 Hz, 1H), 8.29 (d, J=5.2 Hz, 1H), 8.01 (s, 1H), 7.62 (d, J=2.0 Hz, 1H), 6.99 (dd, J=5.2 Hz, 1.1 Hz, 1H), 5.01 (dd, J=8.0 Hz, 5.1 Hz, 1H), 4.53 (dd, J=8.0 Hz, 8.0 Hz, 1H), 4.44 (m, 1H), 3.83 (dd, J=10.5 Hz, 8.3 Hz, 1H), 3.76-3.71 (m, 2H), 3.41 (m, 1H), 3.30 (s, 3H), 3.27 (dd, 1H, signal below water signal), 1.84-1.56 (m, 5H), 1.31-1.20 (m, 2H), 1.23 (d, J=7.0 Hz, 3H), 1.20 (d, J=7.0 Hz, 3H), 1.08 (m, 1H), 0.91-0.81 (m, 2H), 0.85 (d, J=6.5 Hz, 3H).

### Example 90

LC/MS: m/z=646.3 [M+H]⁺; rt: 2.23 min (LC/MS-method A). ¹H NMR (400 MHz, DMSO-de) δ ppm: 10.68 (s, 1H), 8.96 (d, J=8.2 Hz, 1H), 8.94 (s, 1H), 8.30 (d, J=5.2 Hz, 1H), 8.01 (s, 1H), 7.63 (d, J=2.0 Hz, 1H), 7.01 (dd, J=5.2 Hz, 1.1 Hz, 1H), 5.02 (dd, J=8.0 Hz, 5.1 Hz, 1H), 4.63 (dd, J=8.2 Hz, 6.8 Hz, 1H), 4.44 (m, 1H), 3.84 (dd, J=10.5 Hz, 8.3 Hz, 1H), 3.78-3.72 (m, 2H), ~3.30 (s, 3H, signal below water signal), 3.29 (dd, 1H, signal below water signal), 2.77 (s, 3H), 1.80-1.59 (m, 5H), 1.30-1.20 (m, 2H), 1.11 (m, 1H), 0.91-0.81 (m, 2H), 0.85 (d, J=6.5 Hz, 3H).

### Example 91

LC/MS: m/z=583.3 [M+H]⁺; rt: 2.30 min (LC/MS-method A). ¹H NMR (400 MHz, DMSO-de) δ ppm: 10.75 (s, 1H), 8.95 (s, 1H), 8.30 (d, J=5.2 Hz, 1H), 8.22 (d, J=9.0 Hz, 1H), 8.00 (s, 1H), 7.63 (d, J=2.0 Hz, 1H), 6.99 (dd, J=5.2 Hz, 1.1 Hz, 1H), 5.00 (dd, J=8.1 Hz, 5.1 Hz, 1H), 4.63 (dd, J=9.0 Hz, 6.9 Hz, 1H), 4.44 (m, 1H), 3.83 (dd, J=10.5 Hz, 8.4 Hz, 1H), 3.77-3.71 (m, 2H), ~3.30 (s, 3H, signal below water signal), 3.27 (dd, 1H, signal below water signal), 2.75 (s, 3H), 1.82-1.63 (m, 5H), 1.29-1.13 (m, 2H), 1.02 (m, 1H), 0.91-0.82 (m, 2H), 0.83 (d, J=6.5 Hz, 3H).

### Example 92

LC/MS: m/z=594.3 [M+H]⁺; rt: 2.16 min (LC/MS-method A). ¹H NMR (400 MHz, DMSO-de) δ ppm: 10.80 (s, 1H), 9.49 (s, 1H), 8.49 (d, J=8.8 Hz, 1H), 8.30 (d, J=5.2 Hz, 1H), 8.00 (s, 1H), 7.63 (d, J=2.0 Hz, 1H), 7.00 (dd, J=5.2 Hz, 1.1 Hz, 1H), 5.01 (dd, J=8.1 Hz, 5.0 Hz, 1H), 4.64 (dd, J=8.8 Hz, 7.3 Hz, 1H), 4.44 (m, 1H), 3.83 (dd, J=10.5 Hz, 8.4 Hz, 1H), 3.77-3.71 (m, 2H), ~3.30 (s, 3H, signal below water signal), 3.28 (dd, 1H, signal below water signal), 1.88-1.63 (m, 5H), 1.30-1.15 (m, 2H), 1.04 (m, 1H), 0.93-0.82 (m, 2H), 0.84 (d, J=6.5 Hz, 3H).

### Example 93

LC/MS: m/z=568.4 [M+H]⁺; rt: 2.01 min (LC/MS-method A). ¹H NMR (400 MHz, DMSO-de) δ ppm: 10.71 (s, 1H), 8.75 (d, J=8.5 Hz, 1H), 8.30 (d, J=5.2 Hz, 1H), 8.01 (s, 1H), 7.63 (d, J=2.0 Hz, 1H), 7.00 (dd, J=5.2 Hz, 1.1 Hz, 1H), 5.01 (dd, J=8.0 Hz, 5.1 Hz, 1H), 4.61 (dd, J=8.5 Hz, 8.0 Hz, 1H), 4.45 (s, 3H), 4.44 (m, 1H), 3.83 (dd, J=10.5 Hz, 8.4 Hz, 1H), 3.77-3.72 (m, 2H), ~3.30 (s, 3H, signal below water signal), 3.28 (dd, 1H, signal below water signal), 1.81-1.61 (m, 5H), 1.30-1.16 (m, 2H), 1.04 (m, 1H), 0.91-0.82 (m, 2H), 0.84 (d, J=6.5 Hz, 3H).

### Example 94

LC/MS: m/z=655.2 [M+H]⁺; rt: 2.56 min (LC/MS-method A). ¹H NMR (400 MHz, DMSO-de) δ ppm: 10.72 (s, 1H), 8.57 (d, J=8.4 Hz, 1H), 8.30 (d, J=5.2 Hz, 1H), 8.02 (s, 1H), 7.72 (d, J=8.3 Hz, 1H), 7.69 (d, J=8.3 Hz, 1H), 7.63 (d, J=2.0 Hz, 1H), 7.00 (dd, J=5.2 Hz, 1.1 Hz, 1H), 5.02 (dd, J=8.1 Hz, 5.0 Hz, 1H), 4.63 (dd, J=8.4 Hz, 7.1 Hz, 1H), 4.44 (m, 1H), 3.84 (dd, J=10.5 Hz, 8.4 Hz, 1H), 3.77-3.72 (m, 2H), ~3.30 (s, 3H, signal below water signal), 3.29 (dd, 1H, signal below water signal), 2.44 (s, 3H), 1.81-1.61 (m, 5H), 1.29-1.17 (m, 2H), 1.07 (m, 1H), 0.91-0.82 (m, 2H), 0.84 (d, J=6.5 Hz, 3H).

### Example 95

LC/MS: m/z=612.3 [M+H]⁺; rt: 2.36 min (LC/MS-method A). ¹H NMR (400 MHz, DMSO-de) δ ppm: 10.75 (s, 1H), 8.81 (d, J=8.4 Hz, 1H), 8.79 (d, J=0.6 Hz, 1H), 8.30 (d, J=5.2 Hz, 1H), 8.02 (bs, 1H), 7.63 (d, J=2.0 Hz, 1H), 7.01 (dd, J=5.2 Hz, 1.1 Hz, 1H), 5.02 (dd, J=8.0 Hz, 5.1 Hz, 1H), 4.64 (dd, J=8.4 Hz, 7.0 Hz, 1H), 4.44 (m, 1H), 3.84 (dd, J=10.5 Hz, 8.4 Hz, 1H), 3.77-3.72 (m, 2H), ~3.30 (s, 3H, signal below water signal), 3.29 (m, 1H), 2.36 (s, 3H), 1.80-1.61 (m, 5H), 1.29-1.18 (m, 2H), 1.08 (m, 1H), 0.91-0.83 (m, 2H), 0.84 (d, J=6.5 Hz, 3H).

### Example 96: 4-Cyclopropyl-N-((1S)-2-((4-(cyclopropyl(4,4,4-trifluorobutanamido)-methyl)pyridin-2-yl)amino)-1-(4,4-difluorocyclohexyl)-2-oxoethyl)-1,2,5-oxadiazole-3-carboxamide

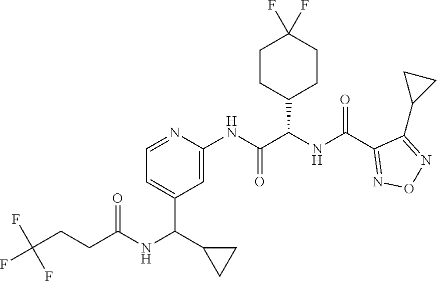 [SMILES: O=C(CCC(F)(F)F)NC(c1ccnc(NC(=O)[C@@H](NC(=O)c2nonc2C2CC2)C2CCC(F)(F)CC2)c1)C1CC1]

Following the procedure described for Example 39, using 47 mg of N-((2-((S)-2-amino-2-(4,4-difluorocyclohexyl)acetamido)pyridin-4-yl)(cyclopropyl)methyl)-4,4,4-trifluorobutanamide (Intermediate 41) and 17 mg of 4-cyclopropyl-1,2,5-oxadiazole-3-carboxylic acid, 41 mg of the title compound was obtained. LC/MS: m/z=599.3 [M+H]⁺; rt: 2.40 min (LC/MS-method A). ¹H NMR (400 MHz, DMSO-d₆) δ ppm 10.83 (s, 1H), 9.30 (d, J=8.0 Hz, 1H), 8.74 (d, J=7.7 Hz, 1H), 8.27 (d, J=5.2 Hz, 1H), 8.10 (s, 1H), 7.12 (dd, J=5.2 Hz, 1.3 Hz, 1H), 4.74 (dd, J=8.1 Hz, 7.7 Hz, 1H), 4.16 (dd, J=8.7 Hz, 8.0 Hz, 1H), 2.50-2.39 (m, 4H), 2.28 (m, 1H), 2.11-1.98 (m, 3H), 1.93-1.66 (m, 4H), 1.51 (m, 1H), 1.37 (m, 1H), 1.15-1.05 (m, 3H), 0.98 (m, 2H), 0.53 (m, 2H), 0.43-0.32 (m, 2H).

### Example 97: (S)-2-(3-Benzylureido)-2-((1r,4S)-4-methylcyclohexyl)-N-(4-(((S)-2-oxo-4-(trifluoromethyl)imidazolidin-1-yl)methyl)pyridin-2-yl)acetamide

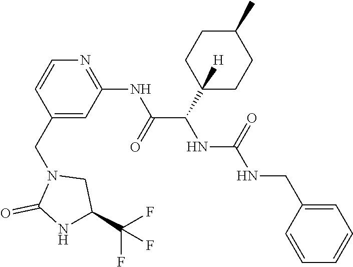

(S)-2-Amino-2-((1r,4S)-4-methylcyclohexyl)-N-(4-(((S)-2-oxo-4-(trifluoromethyl)-imidazolidin-1-yl)methyl)pyridin-2-yl)acetamide HCl salt (Intermediate 2; 20 mg) was dissolved in DCM (1.5 ml). DIPEA (23 μl) and benzyl isocyanate (7 mg) dissolved in DCM (0.5 ml) were added with stirring under Ar. After stirring for 2 h, the mixture stood overnight. Water was added, and the aqueous phase was extracted with DCM (3×). The combined organic phases were dried with sodium sulphate, filtered, and concentrated in vacuo. The residue was purified by preparative RP HPLC (Purosphere® STAR-RP18, 25 mm×250 mm, 10 μm; 30 ml/min, from 95% H₂O/5% ACN to 10% H₂O/90% ACN in 45 min). The pure product containing fractions were combined, the ACN was partly removed and the residue freeze dried to yield 8 mg of the title compound. LC/MS: m/z=547.4 [M+H]⁺; rt: 2.14 min (LC/MS-method A). ¹H NMR (400 MHz, DMSO-d₆) δ ppm: 10.44 (s, 1H), 8.27 (d, J=5.2 Hz, 1H), 7.98 (bs, 1H), 7.64 (d, J=2.0, 1H), 7.34-7.19 (m, 5H), 6.93 (dd, J=5.2 Hz, 1.1 Hz, 1H), 6.54 (t, J=6.0 Hz, 1H), 6.23 (d, J=8.9 Hz, 1H), 4.42 (m, 1H), 4.38 (d, J=16.2 Hz, 1H), 4.34 (dd, J=8.9 Hz, 6.4 Hz, 1H), 4.23 (d, J=16.2 Hz, 1H), 4.21 (d, J=6.0 Hz, 2H), 3.58 (m, 1H), ~3.30 (m, 1H, signal below water signal), 1.69-1.56 (m, 5H), 1.25-1.13 (m, 2H), 1.02 (m, 1H), 0.91-0.79 (m, 2H), 0.84 (d, J=6.5 Hz, 3H).

### Example 98: N-((1S)-2-((4-(2-Methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)-3-methyl-picolinamide (DS2)

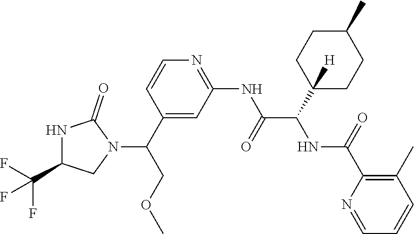

6-Bromo-N-((1S)-2-((4-(2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)-3-methyl-picolinamide (Example 94, DS2; 29 mg) was dissolved in MeOH (3.5 ml) with stirring under Ar. Palladium on charcoal (10%, 2 mg) was added, and the flask was flushed with hydrogen. After stirring for 2 h, under a hydrogen atmosphere (balloon), the mixture was filtered, the filter was washed with MeOH, and the filtrate was concentrated in vacuo. The residue was purified by preparative RP HPLC (Purosphere® STAR-RP18, 25 mm×250 mm, 10 μm; 30 ml/min, from 95% H₂O/5% ACN to 10% H₂O/90% ACN in 45 min). The pure product containing fractions were combined, the ACN was partly removed and the residue freeze dried to yield 12 mg of the title compound. LC/MS: m/z=577.4 [M+H]⁺; rt: 2.34 min (LC/MS-method A). ¹H NMR (400 MHz, DMSO-de) δ ppm: 10.37 (s, 1H), 8.70 (d, J=9.1 Hz, 1H), 8.50 (dd, J=4.7 Hz, 1.0 Hz, 1H), 8.30 (d, J=5.2 Hz, 1H), 8.01 (s, 1H), 7.76 (bd, J=7.8 Hz, 1H), 7.63 (d, J=1.9 Hz, 1H), 7.49 (dd, J=7.8 Hz, 4.6 Hz, 1H), 6.99 (dd, J=5.2 Hz, 1.1 Hz, 1H), 5.01 (dd, J=8.0 Hz, 5.0 Hz, 1H), 4.66 (m, 1H), 4.44 (m, 1H), 3.83 (dd, J=10.5 Hz, 8.3 Hz, 1H), 3.77-3.72 (m, 2H), ~3.29 (m, 1H), ~3.30 (s, 3H, signal below water signal), 2.56 (s, 3H), 1.83-1.62 (m, 5H), 1.30-1.00 (m, 3H), 0.93-0.84 (m, 2H), 0.84 (d, J=6.5 Hz, 3H).

### Example 99: (S)-2-(3-Benzylureido)-2-(4,4-difluorocyclohexyl)-N-(4-(((S)-2-oxo-4-(trifluoromethyl)imidazolidin-1-yl)methyl)pyridin-2-yl)acetamide

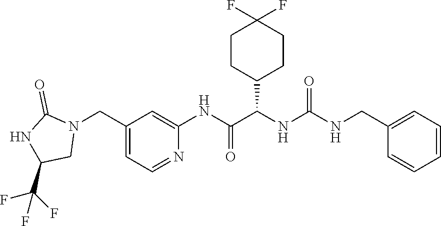 [SMILES: O=C(NCc1ccccc1)N[C@H](C(=O)Nc1cc(CN2C[C@@H](C(F)(F)F)NC2=O)ccn1)C1CCC(F)(F)CC1]

Using (S)-2-amino-2-(4,4-difluorocyclohexyl)-N-(4-(((S)-2-oxo-4-(trifluoromethyl)-imidazolidin-1-yl)methyl)pyridin-2-yl)acetamide HCl salt (Intermediate 4; 25 mg) as starting material analogously to Example 97, the title compound (3 mg) was prepared. LC/MS: m/z=569.3 [M+H]⁺; rt: 1.91 min (LC/MS-method A). ¹H NMR (400 MHz, DMSO-de) δ ppm: 10.57 (s, 1H), 8.28 (d, J=5.2 Hz, 1H), 7.99 (bs, 1H), 7.65 (d, J=2.0, 1H), 7.32-7.20 (m, 5H), 6.94 (dd, J=5.2 Hz, 1.1 Hz, 1H), 6.58 (t, J=6.0 Hz, 1H), 6.32 (d, J=8.9 Hz, 1H), 4.49 (dd, J=8.9 Hz, 6.1 Hz, 1H), 4.42 (m, 1H), 4.38 (d, J=16.2 Hz, 1H), 4.24 (d, J=16.2 Hz, 1H), 4.21 (d, J=6.0 Hz, 2H), 3.58 (m, 1H), ~3.30 (m, 1H, signal below water signal), 2.02 (m, 2H), 1.84-1.65 (m, 5H), 1.46-1.25 (m, 2H).

### Examples 100 and 101: Benzyl ((2R,3R)-3-(tert-butoxy)-1-oxo-1-((4-(((S)-2-oxo-4-(trifluoromethyl)imidazolidin-1-yl)methyl)pyridin-2-yl)amino)butan-2-yl)carbamate and benzyl ((2S,3R)-3-(tert-butoxy)-1-oxo-1-((4-(((S)-2-oxo-4-(trifluoromethyl)-imidazolidin-1-yl)methyl)pyridin-2-yl)amino)butan-2-yl)carbamate

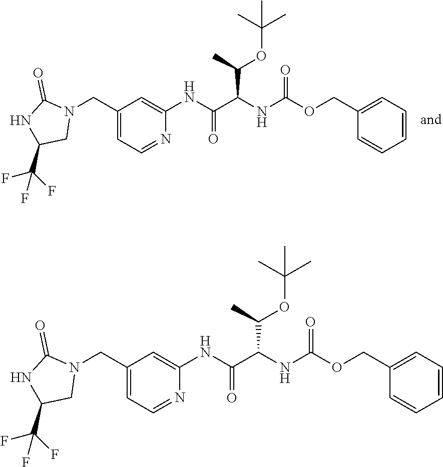 [SMILES: C[C@@H](OC(C)(C)C)[C@@H](NC(=O)OCc1ccccc1)C(=O)Nc1cc(CN2C[C@@H](C(F)(F)F)NC2=O)ccn1 ; C[C@@H](OC(C)(C)C)[C@H](NC(=O)OCc1ccccc1)C(=O)Nc1cc(CN2C[C@@H](C(F)(F)F)NC2=O)ccn1]

To (2S,3R)-2-(Benzyloxycarbonylamino)-3-tert-butoxy-butanoic acid (120 mg) was added DCM (3.5 ml), DMAP (95 mg), and EDC (149 mg) with stirring. After 5 min, (S)-1-((2-aminopyridin-4-yl)methyl)-4-(trifluoromethyl)imidazolidin-2-one (Intermediate 15; 101 mg) was added, and stirring was continued for 1 h. After standing overnight, the mixture was poured onto saturated NaHCO₃-solution. The aqueous phase was extracted with DCM (2×). The combined organic phases were dried with magnesium sulphate, filtered, and concentrated in vacuo. The residue was further purified by preparative RP HPLC (flow: 25 ml/min; gradient: 95% H₂O+0.05% TFA/5% ACN in 45 min to 5% H₂O+0.05% TFA/95% ACN; column: Purosphere® STAR-RP18, 25 mm×250 mm, 10 μm). The fractions containing ex. 107 or 108 were combined separately, the ACN was removed, saturated NaHCO₃ solution was added (pH~7), and the aqueous mixture was extracted with DCM (2×). The combined organic phases were dried over sodium sulphate, filtered and concentrated in vacuo to yield 39 mg of Example 100 and 41 mg of Example 101. The products were separately further purified by silica gel chromatography (silica gel 4 g, DCM/EtOH: 100:0 for 5 min, 100:0 to 90:10 in 30 min, 90:10 for 10 min). The pure compound containing fractions were combined, and concentrated in vacuo. The residue was dissolved in ACN/water and freeze dried to yield 24 mg of Example 100 and 35 mg of Example 101.

### Example 100

LC/MS: m/z=552.3 [M+H]⁺; rt: 2.18 min (LC/MS-method A). ¹H NMR (400 MHz, DMSO-d₆) δ ppm: 10.28 (s, 1H), 8.28 (d, J=5.1 Hz, 1H), 7.97 (s, 1H), 7.65 (d, J=1.9 Hz, 1H), 7.49 (d, J=9.0 Hz, 1H), 7.28-7.38 (m, 5H), 6.93 (dd, J=5.1 Hz, 1.3 Hz, 1H), 5.06 (d, J=12.8 Hz, 1H), 5.02 (d, J=12.8 Hz, 1H), 4.40 (m, 1H), 4.38 (d, J=16.2 Hz, 1H), 4.25 (m, 1H), 4.22 (d, J=16.2 Hz, 1H), 3.89 (m, 1H), 3.58 (m, 1H), ~3.32 (m, 1H), 1.11 (d, J=6.1 Hz, 3H), 1.04 (s, 9H).

### Example 101

LC/MS: m/z=552.3 [M+H]⁺; rt: 2.34 min (LC/MS-method A). ¹H NMR (400 MHz, DMSO-de) δ ppm: 10.32 (s, 1H), 8.28 (d, J=5.1 Hz, 1H), 7.95 (s, 1H), 7.66 (d, J=1.9 Hz, 1H), 7.25-7.41 (m, 5H), 6.95 (dd, J=5.1 Hz, 1.3 Hz, 1H), 6.86 (d, J=9.3 Hz, 1H), 5.07 (s, 2H), 4.41 (m, 1H), 4.41 (d, J=16.2 Hz, 1H), 4.28 (dd, J=9.3 Hz, 3.1 Hz, 1H), 4.20 (d, J=16.2 Hz, 1H), 4.05 (m, 1H), 3.57 (m, 1H), 3.29 (m, 1H), 1.10 (d, J=6.1 Hz, 3H), 1.08 (s, 9H).

### Example 102: N-((2S,3R)-3-(tert-butoxy)-1-oxo-1-((4-(((S)-2-oxo-4-(trifluoromethyl)-imidazolidin-1-yl)methyl)pyridin-2-yl)amino)butan-2-yl)-4-cyclopropyl-1,2,5-oxadiazole-3-carboxamide

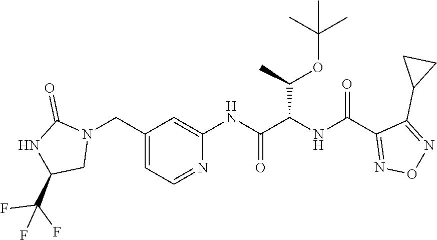 [SMILES: C[C@@H](OC(C)(C)C)[C@H](NC(=O)c1nonc1C1CC1)C(=O)Nc1cc(CN2C[C@@H](C(F)(F)F)NC2=O)ccn1]

To a mixture of (2S,3R)-2-amino-3-(tert-butoxy)-N-(4-(((S)-2-oxo-4-(trifluoromethyl)imidazolidin-1-yl)methyl)pyridin-2-yl)butanamide (Intermediate 27; 12 mg), 4-cyclopropyl-1,2,5-oxadiazole-3-carboxylic acid (5 mg), and DCM (1 ml), DIPEA (24 μl) and T3P (49 μl) were added. After 0.5 h, the mixture was concentrated in vacuo. The residue was purified by preparative HPLC (Agilent Prep C18, 30 mm×100 mm, 5 μm; 40 ml/min, from 97% H₂O/3% ACN to 10% H₂O/90% ACN in 12.5 min). The pure product containing fractions were combined, the ACN was partly removed, and the residue freeze dried to yield 8 mg of the title compound. LC/MS: m/z=554.3 [M+H]⁺; rt: 2.38 min (LC/MS-method A). ¹H NMR (400 MHz, DMSO-d₆) δ: ppm 10.63 (s, 1H), 8.46 (d, J=8.8 Hz, 1H), 8.30 (d, J=5.1 Hz, 1H), 7.96 (s, 1H), 7.66 (d, J=2.0 Hz, 1H), 6.97 (dd, J=5.1, 1.3 Hz, 1H), 4.73 (dd, J=8.8, 2.9 Hz, 1H), 4.43 (d, J=16.1 Hz, 1H), 4.22 (m, 1H), 4.20 (d, J=16.1 Hz, 1H), 3.58 (m, 1H), 3.29 (m, 1H), 2.34 (m, 1H), 1.17 (d, J=6.1 Hz, 3H), 1.15 (m, 2H), 1.10 (s, 9H), 1.00 (m, 2H).

### Example 103: N-((2S,3R)-3-(tert-butoxy)-1-oxo-1-((4-(((S)-2-oxo-4-(trifluoromethyl)-imidazolidin-1-yl)methyl)pyridin-2-yl)amino)butan-2-yl)-1-methyl-1H-pyrazole-5-carboxamide

Following the procedure of Example 102, and using (2S,3R)-2-amino-3-(tert-butoxy)-N-(4-(((S)-2-oxo-4-(trifluoromethyl)imidazolidin-1-yl)methyl)pyridin-2-yl)butan-amide (Intermediate 27; 12 mg), 12 mg of the title compound was obtained. LC/MS: m/z=526.2 [M+H]⁺; rt: 1.88 min (LC/MS-method A). ¹H NMR (400 MHz, DMSO-d₆) δ ppm: 10.46 (s, 1H), 8.29 (d, J=5.1 Hz, 2H), 8.01 (d, J=8.7 Hz, 1H), 7.97 (s, 1H), 7.65 (d, J=2.0 Hz 1H), 7.49 (d, J=2.0 Hz, 1H), 6.99 (d, J=2.0 Hz, 1H), 6.96 (dd, J=5.1, 1.3 Hz, 1H),) 4.71 (dd, J=8.7, 3.7 Hz, 1H), ~4.42 (m, 1H), 4.40 (d, J=16.1 Hz, 1H), 4.21 (d, J=16.1 Hz, 1H), 4.14 (m, 1H), 4.04 (s, 3H), 3.58 (m, 1H), ~3.30 (m, 1H), 1.15 (d, J=6.1 Hz, 3H), 1.14 (s, 9H).

### Example 104: Tert-butyl ((S)-1,1-dicyclopropyl-3-oxo-3-((4-(((S)-2-oxo-4-(trifluoro-methyl)imidazolidin-1-yl)methyl)pyridin-2-yl)amino)propan-2-yl)carbamate

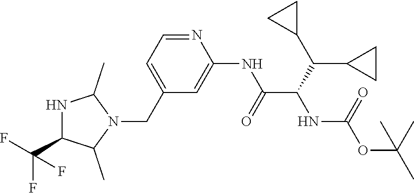 [SMILES: CC1N[C@H](C(F)(F)F)C(C)N1Cc1ccnc(NC(=O)[C@@H](NC(=O)OC(C)(C)C)C(C2CC2)C2CC2)c1]

Tert-butyl ((S)-1-((4-((((S)-2-amino-3,3,3-trifluoropropyl)amino)methyl)pyridin-2-yl)amino)-3,3-dicyclopropyl-1-oxopropan-2-yl)carbamate (Intermediate 28; 181 mg) was dissolved in THE (15 ml). After the addition of DIPEA (0.26 ml), the mixture was heated to 65° C. for 5 min. Then CDI (181 mg) was added, and stirring was continued overnight at 65° C. At RT the mixture was concentrated and MeOH (2.5 ml) and NaOH solution (2 M, 0.5 ml) were added. After stirring for 5 min, the mixture was poured on saturated NaHCO₃ solution. The aqueous mixture was extracted with DCM (2×). The combined organic phases were dried over sodium sulphate, filtered and concentrated in vacuo. The residue was further purified by preparative RP HPLC (Agilent Prep C18 (10 μm, 21.2 mm×250 mm); 50 ml/min, from 90% H₂O/10% ACN to 90% ACN/10% H₂O in 12.5 min). The pure product containing fractions were combined, the ACN was partly removed, and the residue freeze dried to yield 119 mg of the title compound. LC/MS: m/z=512.3 [M+H]⁺; rt: 2.14 min (LC/MS-method A). ¹H NMR (400 MHz, DMSO-d₆) δ ppm: 10.36 (s, 1H), 8.26 (d, J=5.1 Hz, 1H), 7.96 (s, 1H), 7.65 (d, J=1.9 Hz, 1H), 6.93 (dd, J=5.1 Hz, 1.2 Hz, 1H), 6.91 (d, 1H), 4.42 (d, J=16.2 Hz, 1H), 4.39 (m, 2H), 4.19 (d, J=16.2 Hz, 1H), 3.58 (m, 1H), 3.30 (m, 1H), 1.40 (s, 9H), 0.89 (m, 1H), 0.77 (m, 1H), 0.56 (m, 1H), 0.44 (m, 1H), 0.33 (m, 1H), 0.02-0.29 (m, 6H).

### Example 105: N-((1S)-2-((4-(cyclopropyl(4,4,4-trifluorobutanamido)methyl)pyridin-2-yl)amino)-1-(4,4-difluorocyclohexyl)-2-oxoethyl)oxazole-2-carboxamide

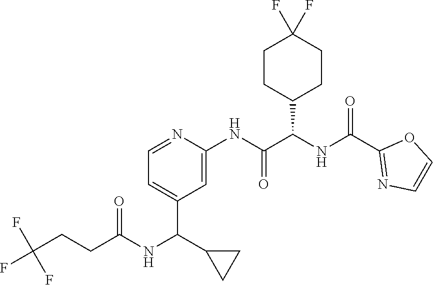 [SMILES: O=C(CCC(F)(F)F)NC(c1ccnc(NC(=O)[C@@H](NC(=O)c2ncco2)C2CCC(F)(F)CC2)c1)C1CC1]

Following the procedure described for Example 39, using 40 mg N-((2-((S)-2-amino-2-(4,4-difluorocyclohexyl)acetamido)pyridin-4-yl)(cyclopropyl)methyl)-4,4,4-trifluorobutanamide (Intermediate 41) and 11 mg oxazole-2-carboxylic acid, 39 mg of the title compound was obtained. LC/MS: m/z=558.3 [M+H]⁺; rt: 1.99 min (LC/MS-method A). ¹H NMR (400 MHz, DMSO-d₆) δ ppm 10.86 (s, 1H), 8.81 (d, J=8.4 Hz, 1H), 8.74 (d, J=7.7 Hz, 1H), 8.35 (d, J=0.6 Hz, 1H), 8.27 (d, J=5.2 Hz, 1H), 8.07 (bs, 1H), 7.49 (d, J=0.6 Hz, 1H), 7.13 (dd, J=5.2 Hz, 1.1 Hz, 1H), 4.65 (dd, J=8.4 Hz, 8.2 Hz, 1H), 4.16 (dd, J=9.0 Hz, 7.7 Hz, 1H), 2.48-2.40 (m, 4H), 2.12-2.00 (m, 3H), 1.89-1.69 (m, 4H), 1.45 (m, 1H), 1.31 (m, 1H), 1.09 (m, 1H), 0.52 (m, 2H), 0.37 (m, 2H).

### Example 106: N-((1S)-2-((4-(cyclopropyl(4,4,4-trifluorobutanamido)methyl)pyridin-2-yl)amino)-1-((1r,4S)-4-methylcyclohexyl)-2-oxoethyl)-1-isopropyl-1H-pyrazole-5-carboxamide

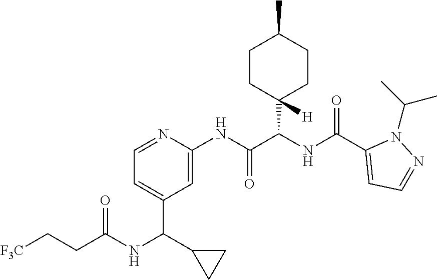 [SMILES: CC(C)n1nccc1C(=O)N[C@H](C(=O)Nc1cc(C(NC(=O)CCC(F)(F)F)C2CC2)ccn1)[C@H]1CC[C@H](C)CC1]

Following the procedure described for Example 33, using 33 mg of N-((2-((S)-2-amino-2-((1r,4S)-4-methylcyclohexyl)acetamido)pyridin-4-yl)(cyclopropyl)methyl)-4,4,4-trifluorobutanamide (Intermediate 42) and 13 mg of 2-isopropylpyrazole-3-carboxylic acid, 40 mg of the title compound was obtained. LC/MS: m/z=577.4 [M+H]⁺; rt: 2.48 min (LC/MS-method A). ¹H NMR (400 MHz, DMSO-d₆) δ ppm 10.76 (s, 1H), 8.75 (d, J=7.7 Hz, 1H), 8.47 (d, J=8.0 Hz, 1H), 8.26 (d, J=5.3 Hz, 1H), 8.05 (bs, 1H), 7.50 (d, J=2.0 Hz, 1H), 7.14 (dd, J=5.3 Hz, 1.1 Hz, 1H), 6.94 (d, J=2.0 Hz, 1H), 5.37 (m, 1H), 4.48 (dd, J=8.4 Hz, 8.0 Hz, 1H), 4.16 (dd, J=9.0 Hz, 7.7 Hz, 1H), 2.49-2.40 (m, 4H), 1.85-1.57 (m, 5H), 1.35 (d, J=6.6 Hz, 3H), 1.33 (d, J=6.6 Hz, 3H), 1.33-1.19 (m, 2H), 1.12-1.03 (m, 2H), 0.92-0.82 (m, 2H), 0.85 (d, J=6.5 Hz, 3H), 0.53 (m, 2H), 0.41 (m, 1H), 0.34 (m, 1H).

### Example 107: N—((S)-2-((4-((R)-cyclopropyl(4,4,4-trifluorobutanamido)methyl)pyridin-2-yl)amino)-1-(4,4-difluorocyclohexyl)-2-oxoethyl)-1-methyl-1H-pyrazole-5-carboxamide

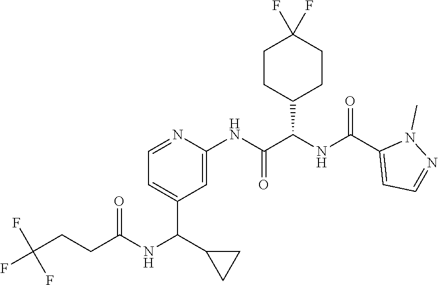 [SMILES: Cn1nccc1C(=O)N[C@H](C(=O)Nc1cc(C(NC(=O)CCC(F)(F)F)C2CC2)ccn1)C1CCC(F)(F)CC1]

Following the procedure described for Example 33, using 33 mg of N-((2-((S)-2-amino-2-(4,4-difluorocyclohexyl)acetamido)pyridin-4-yl)(cyclopropyl)methyl)-4,4,4-trifluorobutanamide (Intermediate 41) and 10 mg of 2-methylpyrazole-3-carboxylic acid, 35 mg of the title compound was obtained. LC/MS: m/z=571.3 [M+H]⁺; rt: 1.99 min (LC/MS-method A). ¹H NMR (400 MHz, DMSO-d₆) δ ppm 11.02 (s, 1H), 8.78 (d, J=7.7 Hz, 1H), 8.64 (d, J=7.9 Hz, 1H), 8.29 (d, J=5.3 Hz, 1H), 8.05 (bs, 1H), 7.47 (d, J=2.0 Hz, 1H), 7.18 (dd, J=5.3 Hz, 1.1 Hz, 1H), 7.06 (d, J=2.0 Hz, 1H), 4.62 (dd, J=8.8 Hz, 7.9 Hz, 1H), 4.16 (dd, J=9.0 Hz, 7.7 Hz, 1H), 4.02 (s, 3H), 2.49-2.40 (m, 4H), 2.10-2.00 (m, 3H), 1.93-1.68 (m, 4H), 1.49 (m, 1H), 1.35 (m, 1H), 1.09 (m, 1H), 0.53 (m, 2H), 0.42 (m, 1H), 0.35 (m, 1H).

### Example 108: N-((1S)-2-((4-(cyclopropyl(4,4,4-trifluorobutanamido)methyl)pyridin-2-yl)amino)-1-(4,4-difluorocyclohexyl)-2-oxoethyl)-1-ethyl-1H-pyrazole-5-carboxamide

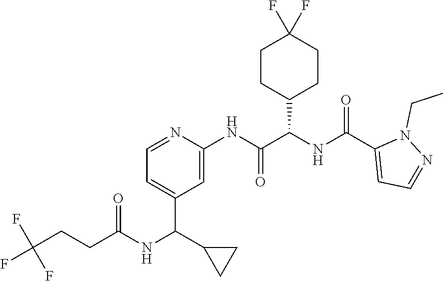 [SMILES: CCn1nccc1C(=O)N[C@H](C(=O)Nc1cc(C(NC(=O)CCC(F)(F)F)C2CC2)ccn1)C1CCC(F)(F)CC1]

Following the procedure described for Example 33, using 33 mg of N-((2-((S)-2-amino-2-(4,4-difluorocyclohexyl)acetamido)pyridin-4-yl)(cyclopropyl)methyl)-4,4,4-trifluorobutanamide (Intermediate 41) and 11 mg of 2-ethylpyrazole-3-carboxylic acid, 39 mg of the title compound was obtained. LC/MS: m/z=585.3 [M+H]⁺; rt: 2.11 min (LC/MS-method A). ¹H NMR (400 MHz, DMSO-d₆) δ ppm 10.94 (s, 1H), 8.77 (d, J=7.7 Hz, 1H), 8.63 (d, J=7.9 Hz, 1H), 8.28 (d, J=5.3 Hz, 1H), 8.06 (bs, 1H), 7.49 (d, J=2.0 Hz, 1H), 7.17 (dd, J=5.3 Hz, 1.1 Hz, 1H), 7.01 (d, J=2.0 Hz, 1H), 4.62 (dd, J=8.7 Hz, 7.9 Hz, 1H), 4.45 (q, J=7.1 Hz, 2H), 4.16 (dd, J=9.0 Hz, 7.7 Hz, 1H), 2.49-2.40 (m, 4H), 2.10-1.99 (m, 3H), 1.93-1.68 (m, 4H), 1.49 (m, 1H), 1.36 (m, 1H), 1.27 (t, J=7.1 Hz, 3H), 1.09 (m, 1H), 0.53 (m, 2H), 0.42 (m, 1H), 0.35 (m, 1H).

### Example 109: N-((1S)-2-((4-(cyclopropyl(4,4,4-trifluorobutanamido)methyl)pyridin-2-yl)amino)-1-(4,4-difluorocyclohexyl)-2-oxoethyl)oxazole-4-carboxamide

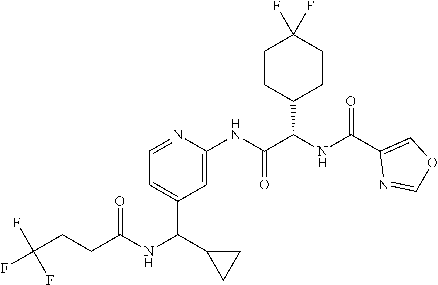 [SMILES: O=C(CCC(F)(F)F)NC(c1ccnc(NC(=O)[C@@H](NC(=O)c2cocn2)C2CCC(F)(F)CC2)c1)C1CC1]

Following the procedure described for Example 33, using 33 mg of N-((2-((S)-2-amino-2-(4,4-difluorocyclohexyl)acetamido)pyridin-4-yl)(cyclopropyl)methyl)-4,4,4-trifluorobutanamide (Intermediate 41) and 9 mg of oxazole-4-carboxylic acid, 34 mg of the title compound was obtained. LC/MS: m/z=558.3 [M+H]⁺; rt: 2.05 min (LC/MS-method A). ¹H NMR (400 MHz, DMSO-de) δ ppm 10.84 (s, 1H), 8.73 (d, J=7.8 Hz, 1H), 8.71 (d, J=1.0 Hz, 1H), 8.56 (d, J=1.0 Hz, 1H), 8.26 (d, J=5.2 Hz, 1H), 8.13 (d, J=8.7 Hz, 1H), 8.08 (bs, 1H), 7.12 (dd, J=5.2 Hz, 1.1 Hz, 1H), 4.72 (dd, J=8.7 Hz, 7.9 Hz, 1H), 4.16 (dd, J=9.0 Hz, 7.8 Hz, 1H), 2.49-2.40 (m, 4H), 2.08-1.99 (m, 3H), 1.86-1.70 (m, 4H), 1.43 (m, 1H), 1.29 (m, 1H), 1.09 (m, 1H), 0.52 (m, 2H), 0.39 (m, 1H), 0.34 (m, 1H).

### Example 110: N-((1S)-2-((4-(cyclopropyl(4,4,4-trifluorobutanamido)methyl)pyridin-2-yl)amino)-1-(4,4-difluorocyclohexyl)-2-oxoethyl)-1-isopropyl-1H-pyrazole-5-carboxamide

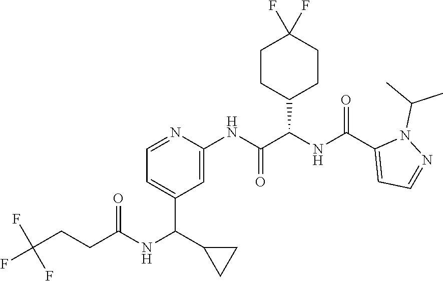 [SMILES: CC(C)n1nccc1C(=O)N[C@H](C(=O)Nc1cc(C(NC(=O)CCC(F)(F)F)C2CC2)ccn1)C1CCC(F)(F)CC1]

Following the procedure described for Example 33, using 33 mg of N-((2-((S)-2-amino-2-(4,4-difluorocyclohexyl)acetamido)pyridin-4-yl)(cyclopropyl)methyl)-4,4,4-trifluorobutanamide (Intermediate 41) and 12 mg of 2-methylpyrazole-3-carboxylic acid, 37 mg of the title compound was obtained. LC/MS: m/z=599.3 [M+H]⁺; rt: 2.23 min (LC/MS-method A). ¹H NMR (400 MHz, DMSO-d₆) δ ppm 10.86 (s, 1H), 8.76 (d, J=7.7 Hz, 1H), 8.60 (d, J=7.9 Hz, 1H), 8.28 (d, J=5.3 Hz, 1H), 8.06 (bs, 1H), 7.51 (d, J=2.0 Hz, 1H), 7.15 (dd, J=5.3 Hz, 1.1 Hz, 1H), 6.94 (d, J=2.0 Hz, 1H), 5.36 (m, 1H), 4.61 (dd, J=8.6 Hz, 7.9 Hz, 1H), 4.16 (dd, J=9.0 Hz, 7.7 Hz, 1H), 2.49-2.40 (m, 4H), 2.09-1.98 (m, 3H), 1.93-1.68 (m, 4H), 1.49 (m, 1H), 1.36 (m, 1H), 1.36 (d, J=6.6 Hz, 3H), 1.34 (d, J=6.6 Hz, 3H), 1.09 (m, 1H), 0.53 (m, 2H), 0.41 (m, 1H), 0.35 (m, 1H).

### Example 111: N-((1S)-2-((4-(cyclopropyl(4,4,4-trifluorobutanamido)methyl)pyridin-2-yl)amino)-1-(4,4-difluorocyclohexyl)-2-oxoethyl)-1-methyl-1H-imidazole-2-carboxamide

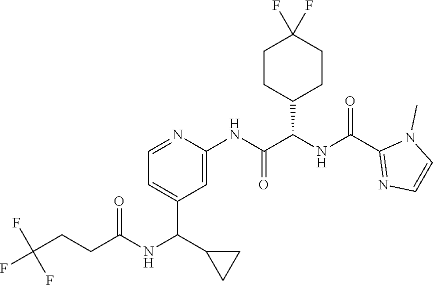 [SMILES: Cn1ccnc1C(=O)N[C@H](C(=O)Nc1cc(C(NC(=O)CCC(F)(F)F)C2CC2)ccn1)C1CCC(F)(F)CC1]

Following the procedure described for Example 33, using 33 mg of N-((2-((S)-2-amino-2-(4,4-difluorocyclohexyl)acetamido)pyridin-4-yl)(cyclopropyl)methyl)-4,4,4-trifluorobutanamide (Intermediate 41) and 10 mg of 1-methylimidazole-2-carboxylic acid, 35 mg of the title compound was obtained. LC/MS: m/z=571.3 [M+H]⁺; rt: 2.03 min (LC/MS-method A). ¹H NMR (400 MHz, DMSO-d₆) δ ppm 10.95 (s, 1H), 8.76 (d, J=7.7 Hz, 1H), 8.73 (bd, J~8 Hz, 1H), 8.28 (d, J=5.2 Hz, 1H), 8.07 (bs, 1H), 7.57 (bs, 1H), 7.32 (bs, 1H), 7.17 (dd, J=5.2 Hz, 1.1 Hz, 1H), 4.71 (dd, J=7.9 Hz, 7.9 Hz, 1H), 4.15 (dd, J=9.1 Hz, 7.7 Hz, 1H), 3.96 (s, 3H), 2.49-2.40 (m, 4H), 2.10-2.00 (m, 3H), 1.90-1.72 (m, 4H), 1.46 (m, 1H), 1.34 (m, 1H), 1.09 (m, 1H), 0.53 (m, 2H), 0.41 (m, 1H), 0.34 (m, 1H).

### Example 112: N-((1S)-2-((4-(2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)-1-methyl-1H-imidazole-2-carboxamide (DS2)

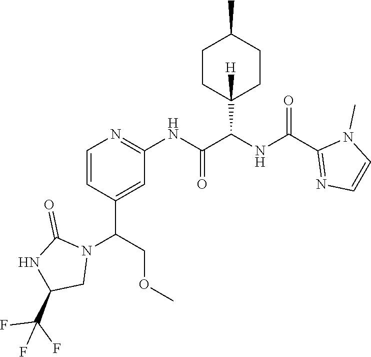

To a mixture of (2S)-2-amino-N-(4-((R or S)-2-methoxy-1-((S)-2-oxo-4-(trifluoromethyl)imidazolidin-1-yl)ethyl)pyridin-2-yl)-2-((1r,4S)-4-methylcyclohexyl)-acetamide hydrochloride salt (Intermediate 18, DS2; 20 mg), 1-methylimidazole-2-carboxylic acid (8 mg), DCM (1 ml), DIPEA (49 μl) and T3P (101 μl) were added. After 30 min the mixture was concentrated in vacuo, and the residue was purified by preparative RP HPLC (Agilent Prep C18, 30 mm×100 mm, 5 μm; 40 ml/min, from 97% H₂O/3% ACN to 10% H₂O/90% ACN in 12.5 min). The pure product containing fractions were combined, the ACN was partly removed, and the residue freeze dried to yield 22 mg of the title compound. LC/MS: m/z=566.3 [M+H]⁺; rt: 2.05 min (LC/MS-method A). ¹H NMR (400 MHz, DMSO-de) δ ppm: 10.74 (s, 1H), 8.29 (d, J=5.2 Hz, 1H), 8.21 (d, J=9.1 Hz, 1H), 8.00 (bs, 1H), 7.63 (d, J=1.9 Hz, 1H), 7.38 (d, J=0.8 Hz, 1H), 7.02 (d, J=0.8 Hz, 1H), 6.99 (dd, J=5.2 Hz, 1.3 Hz, 1H), 5.00 (dd, J=8.1 Hz, 5.0 Hz, 1H), 4.56 (dd, J=9.1 Hz, 8.1 Hz, 1H), 4.44 (m, 1H), 3.93 (s, 3H), 3.83 (dd, J=10.5 Hz, 8.1 Hz, 1H), 3.77-3.72 (m, 2H), 3.31 (s, 3H, signal below water signal), ~3.30 (m, 1H, signal below water signal), 1.86-1.82 (m 5H), 1.25 (m, 1H), 1.17 (m, 1H), 1.01 (m, 1H), 0.91-0.83 (m 2H), 0.84 (d, J=6.5 Hz, 3H).

Using (2S)-2-amino-N-(4-((R or S)-2-methoxy-1-((S)-2-oxo-4-(trifluoromethyl)-imidazolidin-1-yl)ethyl)pyridin-2-yl)-2-((1r,4S)-4-methylcyclohexyl)acetamide hydrochloride salt (Intermediate 18, DS2) as starting material analogously to Example 112, were prepared:

| Ex. | Acid | Structure | Name | Yield |
|---|---|---|---|---|
| 113 | | | (2S)-2-(2-(3,5-dimethyl- | 21 mg, |
| | | | isoxazol-4-yl)acetamido)- | 75% |
| | | | N-(4-(2-methoxy-1-((S)-2- | |
| | | | oxo-4-(trifluoromethyl)- | |
| | | | imidazolidin-1-yl)ethyl)- | |
| | | | pyridin-2-yl)-2-((1r,4S)-4- | |
| | | | methylcyclohexyl)- | |
| | | | acetamide (DS2) | |
| 114 | | | (2S)-N-(4-(2-methoxy-1- | 12 mg, |
| | | | ((S)-2-oxo-4- | 45% |
| | | | (trifluoromethyl)- | |
| | | | imidazolidin-1-yl)ethyl)- | |
| | | | pyridin-2-yl)-2-(2-(5-methyl- | |
| | | | 1H-pyrazol-3- | |
| | | | yl)acetamido)-2-((1r,4S)-4- | |
| | | | methylcyclo- | |
| | | | hexyl)acetamide (DS2) | |
| 115 | | | 1-isopropyl-N-((1S)-2-((4- | 14 mg, |
| | | | (2-methoxy-1-((S)-2-oxo-4- | 43% |
| | | | (trifluoromethyl)imidazolidin- | |
| | | | 1-yl)ethyl)pyridin-2- | |
| | | | yl)amino)-1-((1r,4S)-4- | |
| | | | methylcyclohexyl)-2- | |
| | | | oxoethyl)-1H-imidazole-2- | |
| | | | carboxamide (DS2) | |

### Example 113

LC/MS: m/z=595.3 [M+H]⁺; rt: 2.04 min (LC/MS-method A). ¹H NMR (400 MHz, DMSO-d₆) δ ppm: 10.52 (s, 1H), 8.27 (d, J=5.2 Hz, 1H), 8.22 (d, J=8.3 Hz, 1H), 7.99 (bs, 1H), 7.63 (d, J=1.9 Hz, 1H), 6.98 (dd, J=5.2 Hz, 1.3 Hz, 1H), 5.01 (dd, J=8.2 Hz, 5.1 Hz, 1H), 4.44 (m, 1H), 4.41 (dd, J=8.3 Hz, 7.1 Hz, 1H), 3.83 (dd, J=10.5 Hz, 8.2 Hz, 1H), 3.77-3.72 (m, 2H), 3.34-3.24 (6H, signals below water signal), 2.28 (s, 3H), 2.11 (s, 3H), 1.74-1.52 (m 5H), 1.25 (m, 1H), 1.15 (m, 1H), 1.05 (m, 1H), 0.89-0.80 (m 2H), 0.84 (d, J=6.5 Hz, 3H).

### Example 114

LC/MS: m/z=580.3 [M+H]⁺; rt: 1.88 min (LC/MS-method A). ¹H NMR (400 MHz, DMSO-de) δ ppm: ~12.2 (bs, 1H), 10.52 (s, 1H), 8.28 (d, J=5.2 Hz, 1H), ~8.1 (bd, 1H), 7.99 (bs, 1H), 7.62 (d, J=1.9 Hz, 1H), 6.98 (dd, J=5.2 Hz, 1.3 Hz, 1H), 5.84 (s, 1H), 5.00 (dd, J=8.2 Hz, 5.1 Hz, 1H), 4.44 (m, 1H), 4.43 (dd, 1H), 3.83 (dd, J=10.5 Hz, 8.2 Hz, 1H), 3.77-3.71 (m, 2H), 3.43 (bs, 2H), 3.31 (s, 3H, signal below water signal), 3.27 (dd: J=10.2 Hz, 3.8 Hz, 1H), 2.15 (bs, 3H), 1.72-1.52 (m 5H), 1.24 (m, 1H), 1.15 (m, 1H), 1.02 (m, 1H), 0.88-0.79 (m 2H), 0.84 (d, J=6.5 Hz, 3H).

### Example 115

LC/MS: m/z=594.4 [M+H]⁺; rt: 2.29 min (LC/MS-method A). ¹H NMR (400 MHz, DMSO-d₆) δ ppm: 10.74 (s, 1H), 8.29 (d, J=5.2 Hz, 1H), 8.24 (d, J=9.1 Hz, 1H), 8.00 (bs, 1H), 7.63 (d, J=1.9 Hz, 1H), 7.60 (d, J=1.0 Hz, 1H), 7.05 (d, J=1.0 Hz, 1H), 6.99 (dd, J=5.2 Hz, 1.3 Hz, 1H), 5.57 (m, 1H), 5.00 (dd, J=8.2 Hz, 5.1 Hz, 1H), 4.58 (dd, J=9.1 Hz, 6.8 Hz, 1H), 4.44 (m, 1H), 3.83 (dd, J=10.5 Hz, 8.2 Hz, 1H), 3.77-3.72 (m, 2H), ~3.30 (s, 3H, signal below water signal), 3.28 (dd, 1H, signal below water signal), 1.82-1.62 (m 5H), 1.38 (d, J=6.7 Hz, 3H), 1.36 (d, J=6.7 Hz, 3H), 1.25 (m, 1H), 1.17 (m, 1H), 1.02 (m, 1H), 0.91-0.82 (m 2H), 0.84 (d, J=6.5 Hz, 3H).

### Example 116: N-((2S,3S)-3-(tert-butoxy)-1-oxo-1-((4-(((S)-2-oxo-4-(trifluoromethyl)-imidazolidin-1-yl)methyl)pyridin-2-yl)amino)butan-2-yl)-4-cyclopropyl-1,2,5-oxadiazole-3-carboxamide

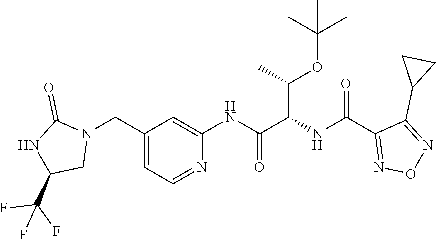 [SMILES: C[C@H](OC(C)(C)C)[C@H](NC(=O)c1nonc1C1CC1)C(=O)Nc1cc(CN2C[C@@H](C(F)(F)F)NC2=O)ccn1]

Following the procedure of Example 102, and using (2S,3S)-2-amino-3-(tert-butoxy)-N-(4-(((S)-2-oxo-4-(trifluoromethyl)imidazolidin-1-yl)methyl)pyridin-2-yl)butanamide (Intermediate 29; 21 mg), 14 mg of the title compound was obtained. LC/MS: m/z=554.3 [M+H]⁺; rt: 2.24 min (LC/MS-method A). ¹H NMR (400 MHz, DMSO-de) δ ppm: 10.62 (s, 1H), 9.15 (d, J=8.5 Hz, 1H), 8.30 (d, J=5.2 Hz, 1H), 7.99 (bs, 1H), 7.65 (d, J=1.9 Hz, 1H), 6.95 (dd, J=5.2 Hz, 1.3 Hz, 1H), 4.75 (dd, J=8.5 Hz, 7.8 Hz, 1H), 4.43 (d, J=16.2 Hz, 1H), 4.41 (m, 1H), 4.19 (d, J=16.2 Hz, 1H), 4.10 (m, 1H), 3.58 (dd, J=10.0 Hz, 10.0 Hz, 1H), 3.29 (dd, 1H, signal below water signal), 2.31 (m, 1H), 1.17 (d, J=6.1 Hz, 3H), 1.13 (m, 2H), 1.08 (s, 9H), 0.99 (m, 2H).

### Example 117: N-((1S)-2-((4-(cyclopropyl(4,4,4-trifluorobutanamido)methyl)pyridin-2-yl)amino)-1-(4,4-difluorocyclohexyl)-2-oxoethyl)-1-isopropyl-1H-imidazole-2-carboxamide

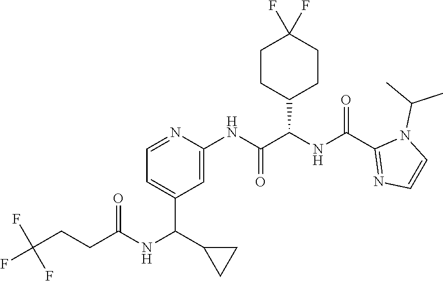 [SMILES: CC(C)n1ccnc1C(=O)N[C@H](C(=O)Nc1cc(C(NC(=O)CCC(F)(F)F)C2CC2)ccn1)C1CCC(F)(F)CC1]

Following the procedure described for Example 33, using 33 mg of N-((2-((S)-2-amino-2-(4,4-difluorocyclohexyl)acetamido)pyridin-4-yl)(cyclopropyl)methyl)-4,4,4-trifluorobutanamide (Intermediate 41) and 16 mg of 1-methylimidazole-2-carboxylic acid, 24 mg of the title compound was obtained. LC/MS: m/z=599.3 [M+H]⁺; rt: 2.25 min (LC/MS-method A). ¹H NMR (400 MHz, DMSO-d₆) δ ppm 10.94 (s, 1H), 8.79 (bd, 1H), 8.76 (d, J=7.7 Hz, 1H), 8.28 (d, J=5.2 Hz, 1H), 8.07 (bs, 1H), 7.81 (bs, 1H), 7.38 (bs, 1H), 7.15 (dd, J=5.2 Hz, 1.3 Hz, 1H), 5.39 (m, 1H), 4.73 (dd, 1H), 4.15 (dd, J=9.1 Hz, 7.7 Hz, 1H), 2.49-2.41 (m, 4H), 2.09-2.00 (m, 3H), 1.88-1.72 (m, 4H), 1.51-1.30 (m 2H), 1.42 (d, J=6.7 Hz, 3H), 1.40 (d, J=6.7 Hz, 3H), 1.09 (m, 1H), 0.53 (m, 2H), 0.41 (m, 1H), 0.34 (m 1H).

### Example 118: 1-methyl-N-((1S)-1-((1r,4S)-4-methylcyclohexyl)-2-oxo-2-((4-((2-oxopyrrolidin-3-yl)oxy)pyridin-2-yl)amino)ethyl)-1H-pyrazole-5-carboxamide

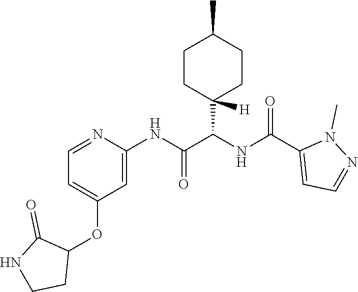 [SMILES: Cn1nccc1C(=O)N[C@H](C(=O)Nc1cc(OC2CCNC2=O)ccn1)[C@H]1CC[C@H](C)CC1]

Following the procedure described for Example 33, using 35 mg (2S)-2-amino-2-((1r,4S)-4-methylcyclohexyl)-N-(4-((2-oxopyrrolidin-3-yl)oxy)pyridin-2-yl)acetamide (Intermediate 43) and 14 mg 1-methylimidazole-2-carboxylic acid, 4 mg of the title compound was obtained. LC/MS: m/z=455.2 [M+H]⁺; rt: 1.62 min (LC/MS-method A). ¹H NMR (400 MHz, DMSO-d₆) δ ppm 8.52 (br t, J=7.03 Hz, 1H), 8.18 (d, J=6.1 Hz, 1H), 7.63 (br s, 1H), 7.46 (dd, J=10.0, 2.0 Hz, 1H), 7.05 (dd, J=2.0, 1.1 Hz, 1H), 6.93 (br d, J=5.5 Hz, 1H), 6.38 (br s, 1H), 5.12 (m, 1H), 4.48 (m, 1H), 4.08 (m, 1H), 4.01 (s, 2H), 3.95 (br s, 1H), 3.77 (s, 3H) 3.26 (m, 2H), 2.61 (m, 1H), 2.04 (m, 1H), 1.81 (m, 2H), 1.68 (br s, 1H), 1.59 (br d, J=13.0 Hz, 1H), 1.28 (br s, 1H), 1.22 (m, 2H), 1.16 (br s, 4H), 1.06 (m, 2H).

### Example 119: 1-isopropyl-N-((1S)-1-((1r,4S)-4-methylcyclohexyl)-2-oxo-2-((4-((2-oxopyrrolidin-3-yl)oxy)pyridin-2-yl)amino)ethyl)-1H-pyrazole-5-carboxamide

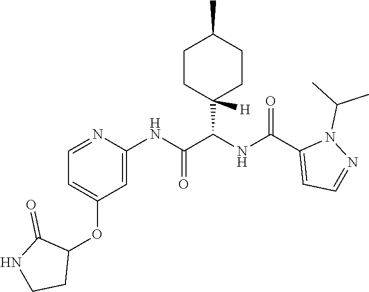 [SMILES: CC(C)n1nccc1C(=O)N[C@H](C(=O)Nc1cc(OC2CCNC2=O)ccn1)[C@H]1CC[C@H](C)CC1]

Following the procedure described for Example 33, using 35 mg (2S)-2-amino-2-((1r,4S)-4-methylcyclohexyl)-N-(4-((2-oxopyrrolidin-3-yl)oxy)pyridin-2-yl)acetamide (Intermediate 43) and 17 mg 1-isopropylimidazole-2-carboxylic acid, 5 mg of the title compound was obtained. LC/MS: m/z=483.2 [M+H]⁺; rt: 1.85 min (LC/MS-method A). ¹H NMR (400 MHz, DMSO-d₆) δ ppm 8.50 (m, 1H), 8.18 (br m, 2H), 7.64 (br s, 1H), 7.50 (d, J=1.9 Hz, 1H), 6.94 (m, 1H), 5.36 (dt, J=12.9, 6.4 Hz, 1H), 5.12 (m, 1H), 4.46 (m, 1H), 4.13 (br s, 1H), 3.88 (br s, 1H), 3.67 (br s, 1H), 3.26 (m, 3H), 2.60 (m, 1H), 2.01 (m, 1H), 1.82 (br t, J=10.1 Hz, 1H), 1.73 (br d, J=19.7 Hz, 2H), 1.59 (br d, J=12.1 Hz, 1H), 1.36 (m, 7H), 1.25 (br dd, J=14.4, 6.8 Hz, 3H), 1.12 (m, 2H), 0.92 (br s, 1H), 0.86 (br d).

Using (2S)-2-amino-N-(4-((R or S)-2-methoxy-1-((S)-2-oxo-4-(trifluoromethyl)-imidazolidin-1-yl)ethyl)pyridin-2-yl)-2-((1r,4S)-4-methylcyclohexyl)acetamide hydrochloride salt (Intermediate 18, DS2) as starting material analogously to example 112, were prepared:

| Ex. | Acid | Structure | Name | Yield |
|---|---|---|---|---|
| 120 | | | N-((S)-2-((4-(2-methoxy- | 19 mg, |
| | | | 1-((S)-2-oxo-4-(trifluoro- | 88% |
| | | | methyl)imidazolidin-1- | |
| | | | yl)ethyl)pyridin-2- | |
| | | | yl)amino)-1-((1r,4S)-4- | |
| | | | methylcyclohexyl)-2- | |
| | | | oxoethyl)-4-methyl- | |
| | | | isoxazole-3-carboxamide | |
| | | | (DS2) | |
| 121 | | | 4-cyclopropyl-N-((S)-2- | 17 mg, |
| | | | ((4-(2-methoxy-1-((S)-2- | 71% |
| | | | oxo-4-(trifluoromethyl)- | |
| | | | imidazolidin-1-yl)ethyl)- | |
| | | | pyridin-2-yl)amino)-1- | |
| | | | ((1r,4S)-4-methylcyclo- | |
| | | | hexyl)-2-oxoethyl)- | |
| | | | isoxazole-3-carboxamide | |
| | | | (DS2) | |
| 122 | | | N-((S)-2-((4-(2-methoxy- | 16 mg, |
| | | | 1-((S)-2-oxo-4-(trifluoro- | 69% |
| | | | methyl)imidazolidin-1- | |
| | | | yl)ethyl)pyridin-2- | |
| | | | yl)amino)-1-((1r,4S)-4- | |
| | | | methylcyclohexyl)-2- | |
| | | | oxoethyl)-4-methyl- | |
| | | | oxazole-5-carboxamide | |
| | | | (DS2) | |
| 123 | | | (S)-N-(4-(2-methoxy-1- | 15 mg, |
| | | | ((S)-2-oxo-4-(trifluoro- | 65% |
| | | | methyl)imidazolidin-1- | |
| | | | yl)ethyl)pyridin-2-yl)-2- | |
| | | | ((1r,4S)-4-methylcyclo- | |
| | | | hexyl)-2-(2-(3-methyl- | |
| | | | isoxazol-5-yl)acetamido)- | |
| | | | acetamide (DS2) | |
| 124 | | | 1-ethyl-N-((S)-2-((4-(2- | 13 mg, |
| | | | methoxy-1-((S)-2-oxo-4- | 55% |
| | | | (trifluoromethyl)imidazolidin- | |
| | | | 1-yl)ethyl)pyridin-2- | |
| | | | yl)amino)-1-((1r,4S)-4- | |
| | | | methylcyclohexyl)-2- | |
| | | | oxoethyl)-1H-pyrazole-5- | |
| | | | carboxamide (DS2) | |
| 125 | | | 3-ethyl-N-((S)-2-((4-(2- | 13 mg, |
| | | | methoxy-1-((S)-2-oxo-4- | 54% |
| | | | (trifluoromethyl)imidazolidin- | |
| | | | 1-yl)ethyl)pyridin-2- | |
| | | | yl)amino)-1-((1r,4S)-4- | |
| | | | methylcyclohexyl)-2- | |
| | | | oxoethyl)isoxazole-4- | |
| | | | carboxamide (DS2) | |
| 126 | | | 3-cyclopropyl-N-((S)-2- | 14 mg, |
| | | | ((4-(2-methoxy-1-((S)-2- | 56% |
| | | | oxo-4-(trifluoromethyl)- | |
| | | | imidazolidin-1- | |
| | | | yl)ethyl)pyridin-2- | |
| | | | yl)amino)-1-((1r,4S)-4- | |
| | | | methylcyclohexyl)-2- | |
| | | | oxoethyl)isoxazole-4- | |
| | | | carboxamide (DS2) | |

### Example 120

LC/MS: m/z=567.4 [M+H]⁺; rt: 2.25 min (LC/MS-method A). ¹H NMR (400 MHz, DMSO-d₆) δ ppm: 10.65 (s, 1H), 8.83 (q, J=1.0 Hz, 1H), 8.55 (d, J=8.4 Hz, 1H), 8.30 (d, J=5.2 Hz, 1H), 8.01 (bs, 1H), 7.63 (d, J=1.9 Hz, 1H), 7.00 (dd, J=5.2 Hz, 1.3 Hz, 1H), 5.01 (dd, J=8.1 Hz, 5.1 Hz, 1H), 4.55 (dd, J=8.4 Hz, 7.7 Hz, 1H), 4.44 (m, 1H), 3.83 (dd, J=10.5 Hz, 8.1 Hz, 1H), 3.77-3.72 (m, 2H), ~3.30 (s, 3H, signal below water signal), 3.28 (dd, 1H, signal below water signal), 2.09 (d, J=1.0 Hz, 3H), 1.83-1.58 (m, 5H), 1.29-1.16 (m, 2H), 1.05 (m, 1H), 0.91-0.82 (m, 2H), 0.84 (d, J=6.5 Hz, 3H).

### Example 121

LC/MS: m/z=593.4 [M+H]⁺; rt: 2.36 min (LC/MS-method A). ¹H NMR (400 MHz, DMSO-de) δ ppm: 10.65 (s, 1H), 8.75 (d, J=0.6 Hz, 1H), 8.59 (d, J=8.4 Hz, 1H), 8.30 (d, J=5.2 Hz, 1H), 8.01 (bs, 1H), 7.63 (d, J=1.9 Hz, 1H), 7.00 (dd, J=5.2 Hz, 1.3 Hz, 1H), 5.01 (dd, J=8.1 Hz, 5.1 Hz, 1H), 4.58 (dd, J=8.4 Hz, 7.8 Hz, 1H), 4.44 (m, 1H), 3.83 (dd, J=10.5 Hz, 8.1 Hz, 1H), 3.77-3.72 (m, 2H), ~3.30 (s, 3H, signal below water signal), 3.29 (dd, 1H, signal below water signal), 1.90 (m, 1H), 1.82-1.58 (m 5H), 1.30-1.17 (m, 2H), 1.06 (m, 1H), 0.91-0.82 (m, 4H), 0.84 (d, J=6.5 Hz, 3H), 0.59 (m 2H).

### Example 122

LC/MS: m/z=567.3 [M+H]⁺; rt: 2.03 min (LC/MS-method A). ¹H NMR (400 MHz, DMSO-d₆) δ ppm: 10.61 (s, 1H), 8.44 (s, 1H), 8.29 (d, J=5.2 Hz, 1H), 8.19 (d, J=8.4 Hz, 1H), 8.00 (bs, 1H), 7.62 (d, J=1.9 Hz, 1H), 6.99 (dd, J=5.2 Hz, 1.3 Hz, 1H), 5.00 (dd, J=8.1 Hz, 5.1 Hz, 1H), 4.49 (dd, J=8.4 Hz, 8.0 Hz, 1H), 4.44 (m, 1H), 3.83 (dd, J=10.5 Hz, 8.1 Hz, 1H), 3.76-3.71 (m, 2H), 3.31 (s, 3H, signal below water signal), 3.28 (dd, 1H, signal below water signal), 2.35 (s, 3H), 1.84-1.57 (m 5H), 1.29-1.15 (m, 2H), 1.03 (m, 1H), 0.93-0.81 (m 2H), 0.84 (d, J=6.5 Hz, 3H).

### Example 123

LC/MS: m/z=581.4 [M+H]⁺; rt: 2.00 min (LC/MS-method A). ¹H NMR (400 MHz, DMSO-de) δ ppm: 10.57 (s, 1H), 8.36 (d, J=8.2 Hz, 1H), 8.28 (d, J=5.2 Hz, 1H), 8.00 (bs, 1H), 7.62 (d, J=1.9 Hz, 1H), 6.98 (dd, J=5.2 Hz, 1.3 Hz, 1H), 6.15 (s, 1H), 5.01 (dd, J=8.1 Hz, 5.1 Hz, 1H), 4.46 (dd, J=8.2 Hz, 7.1 Hz, 1H), 4.44 (m, 1H), 3.83 (dd, J=10.5 Hz, 8.1 Hz, 1H), 3.79-3.71 (m, 4H), ~3.30 (s, 3H, signal below water signal), 3.27 (dd, 1H, signal below water signal), 2.18 (s, 3H), 1.74-1.53 (m 5H), 1.25 (m, 1H), 1.17 (m, 1H), 1.06 (m, 1H), 0.89-0.80 (m 2H), 0.84 (d, J=6.5 Hz, 3H).

### Example 124

LC/MS: m/z=580.4 [M+H]⁺; rt: 2.16 min (LC/MS-method A). ¹H NMR (400 MHz, DMSO-de) δ ppm: 10.59 (s, 1H), 8.43 (d, J=8.0 Hz, 1H), 8.29 (d, J=5.2 Hz, 1H), 8.01 (bs, 1H), 7.62 (d, J=1.9 Hz, 1H), 7.48 (d, J=2.0 Hz, 1H), 6.99 (dd, J=5.2 Hz, 1.3 Hz, 1H), 6.99 (d, J=2.0 Hz, 1H), 5.00 (dd, J=8.1 Hz, 5.1 Hz, 1H), 4.50 (dd, J=8.7 Hz, 8.0 Hz, 1H), 4.44 (m, 1H), 4.44 (q, J=7.1 Hz, 2H), 3.83 (dd, J=10.5 Hz, 8.1 Hz, 1H), 3.76-3.71 (m, 2H), 3.30 (s, 3H, signal below water signal), 3.28 (dd, 1H, signal below water signal), 1.84-1.56 (m 5H), 1.31-1.18 (m, 2H), 1.26 (t, J=7.1 Hz, 3H), 1.06 (m, 1H), 0.92-0.81 (m 2H), 0.85 (d, J=6.5 Hz, 3H).

### Example 125

LC/MS: m/z=581.4 [M+H]⁺; rt: 2.22 min (LC/MS-method A). ¹H NMR (400 MHz, DMSO-de) δ ppm: 10.60 (s, 1H), 9.40 (s, 1H), 8.38 (d, J=8.1 Hz, 1H), 8.29 (d, J=5.2 Hz, 1H), 8.01 (bs, 1H), 7.62 (d, J=1.9 Hz, 1H), 6.99 (dd, J=5.2 Hz, 1.3 Hz, 1H), 5.01 (dd, J=8.1 Hz, 5.1 Hz, 1H), 4.54 (dd, J=8.1 Hz, 8.0 Hz, 1H), 4.44 (m, 1H), 3.83 (dd, J=10.5 Hz, 8.1 Hz, 1H), 3.76-3.71 (m, 2H), ~3.30 (s, 3H, signal below water signal), 3.28 (dd, 1H, signal below water signal), 2.82 (q, J=7.5 Hz, 2H), 1.84-1.56 (m 5H), 1.29-1.13 (m, 2H), 1.16 (t, J=7.5 Hz, 3H), 1.08 (m, 1H), 0.91-0.81 (m 2H), 0.85 (d, J=6.5 Hz, 3H).

### Example 126

LC/MS: m/z=593.4 [M+H]⁺; rt: 2.26 min (LC/MS-method A). ¹H NMR (400 MHz, DMSO-d₆) δ ppm: 10.62 (s, 1H), 9.37 (s, 1H), 8.37 (d, J=8.0 Hz, 1H), 8.29 (d, J=5.2 Hz, 1H), 8.01 (bs, 1H), 7.62 (d, J=1.9 Hz, 1H), 6.99 (dd, J=5.2 Hz, 1.3 Hz, 1H), 5.01 (dd, J=8.1 Hz, 5.1 Hz, 1H), 4.55 (dd, J=8.0 Hz, 8.0 Hz, 1H), 4.44 (m, 1H), 3.83 (dd, J=10.5 Hz, 8.1 Hz, 1H), 3.77-3.71 (m, 2H), ~3.30 (s, 3H, signal below water signal), 3.28 (dd, 1H, signal below water signal), 2.43 (m, 1H), 1.85-1.57 (m 5H), 1.32-1.19 (m, 2H), 1.09 (m, 1H), 0.98 (m, 2H), 0.92-0.83 (m 4H), 0.85 (d, J=6.5 Hz, 3H).

Using (2S,3R)-2-amino-3-(tert-butoxy)-N-(4-(((S)-2-oxo-4-(trifluoromethyl)-imidazolidin-1-yl)methyl)pyridin-2-yl)butanamide (Intermediate 27) as starting material analogously to Example 102, were prepared:

| Ex. | Acid | Structure | Name | Yield |
|---|---|---|---|---|
| 127 | | | N-((2S,3R)-3-(tert- | 12 mg, |
| | | | butoxy)-1-oxo-1-((4-(((S)- | 49% |
| | | | 2-oxo-4-(trifluoromethyl)- | |
| | | | imidazolidin-1-yl)methyl)- | |
| | | | pyridin-2-yl)amino)butan- | |
| | | | 2-yl)-1-isopropyl-1H- | |
| | | | pyrazole-5-carboxamide | |
| 128 | | | N-((2S,3R)-3-(tert- | 14 mg, |
| | | | butoxy)-1-oxo-1-((4-(((S)- | 60% |
| | | | 2-oxo-4-(trifluoromethyl)- | |
| | | | imidazolidin-1-yl)methyl)- | |
| | | | pyridin-2-yl)amino)butan- | |
| | | | 2-yl)-4-methyl-1,2,5-oxa- | |
| | | | diazole-3-carboxamide | |
| 129 | | | N-((2S,3R)-3-(tert- | 15 mg, |
| | | | butoxy)-1-oxo-1-((4-(((S)- | 61% |
| | | | 2-oxo-4-(trifluoromethyl)- | |
| | | | imidazolidin-1-yl)methyl)- | |
| | | | pyridin-2-yl)amino)butan- | |
| | | | 2-yl)-4-ethyl-1,2,5-oxa- | |
| | | | diazole-3-carboxamide | |
| 130 | | | N-((2S,3R)-3-(tert- | 15 mg, |
| | | | butoxy)-1-oxo-1-((4-(((S)- | 59% |
| | | | 2-oxo-4-(trifluoromethyl)- | |
| | | | imidazolidin-1-yl)methyl)- | |
| | | | pyridin-2-yl)amino)butan- | |
| | | | 2-yl)-3-isopropyl- | |
| | | | isoxazole-4-carboxamide | |

### Example 127

LC/MS: m/z=554.3 [M+H]⁺; rt: 2.15 min (LC/MS-method A). ¹H NMR (400 MHz, DMSO-d₆) δ ppm: 10.47 (s, 1H), 8.29 (d, J=5.1 Hz, 1H), 7.97 (s, 1H), 7.94 (d, J=8.8 Hz, 1H), 7.65 (d, J=2.0 Hz 1H), 7.53 (d, J=2.0 Hz, 1H), 6.96 (dd, J=5.1 Hz, 1.3 Hz, 1H), 6.90 (d, J=2.0 Hz, 1H), 5.38 (m, 1H), 4.70 (dd, J=8.7 Hz, 3.7 Hz, 1H), 4.42 (m, 1H), 4.41 (d, J=16.1 Hz, 1H), 4.21 (d, J=16.1 Hz, 1H), 4.14 (m, 1H), 3.58 (dd, =10.0 Hz, 10.0 Hz 1H), 3.29 (dd, 1H, signal below water signal), 1.38 (d, J=6.6 Hz, 3H), 1.36 (d, J=6.6 Hz, 3H), 1.15 (d, J=6.3 Hz, 3H), 1.13 (s, 9H).

### Example 128

LC/MS: m/z=528.3 [M+H]⁺; rt: 2.18 min (LC/MS-method A). ¹H NMR (400 MHz, DMSO-de) δ ppm: 10.64 (s, 1H), 8.35 (d, J=8.7 Hz, 1H), 8.30 (d, J=5.1 Hz, 1H), 7.96 (s, 1H), 7.66 (d, J=2.0 Hz 1H), 6.97 (dd, J=5.1 Hz, 1.3 Hz, 1H), 4.71 (dd, J=8.7 Hz, 2.8 Hz, 1H), 4.42 (m, 1H), 4.42 (d, J=16.1 Hz, 1H), 4.22 (m, 1H), 4.19 (d, J=16.1 Hz, 1H), 3.57 (dd, =10.0 Hz, 10.0 Hz 1H), 3.29 (dd, 1H, signal below water signal), 2.50 (s, 3H, below solvent signal), 1.17 (d, J=6.3 Hz, 3H), 1.10 (s, 9H).

### Example 129

LC/MS: m/z=542.3 [M+H]⁺; rt: 2.34 min (LC/MS-method A). ¹H NMR (400 MHz, DMSO-de) δ ppm: 10.63 (s, 1H), 8.40 (d, J=8.7 Hz, 1H), 8.30 (d, J=5.1 Hz, 1H), 7.96 (s, 1H), 7.66 (d, J=2.0 Hz 1H), 6.97 (dd, J=5.1 Hz, 1.3 Hz, 1H), 4.72 (dd, J=8.7 Hz, 2.9 Hz, 1H), 4.43 (d, J=16.1 Hz, 1H), 4.42 (m, 1H), 4.22 (m, 1H), 4.19 (d, J=16.1 Hz, 1H), 3.58 (dd, =10.0 Hz, 10.0 Hz 1H), 3.29 (dd, 1H, signal below water signal), 2.93 (q, J=7.5 Hz, 2H), 1.26 (t, J=7.5 Hz, 3H), 1.17 (d, J=6.3 Hz, 3H), 1.10 (s, 9H).

### Example 130

LC/MS: m/z=555.3 [M+H]⁺; rt: 2.20 min (LC/MS-method A). ¹H NMR (400 MHz, DMSO-de) δ ppm: 10.39 (s, 1H), 9.45 (s, 1H), 8.29 (d, J=5.1 Hz, 1H), 8.12 (d, J=8.7 Hz, 1H), 7.97 (s, 1H), 7.64 (d, J=2.0 Hz 1H), 6.96 (dd, J=5.1 Hz, 1.3 Hz, 1H), 4.73 (dd, J=8.7 Hz, 3.4 Hz, 1H), 4.41 (m, 1H), 4.41 (d, J=16.1 Hz, 1H), 4.20 (d, J=16.1 Hz, 1H), 4.14 (m, 1H), 3.58 (dd, =10.0 Hz, 10.0 Hz 1H), 3.43 (m, 1H), 3.29 (dd, 1H, signal below water signal), 1.26 (d, J=6.9 Hz, 3H), 1.23 (d, J=6.9 Hz, 3H), 1.14 (d, J=6.3 Hz, 3H), 1.12 (s, 9H).

### Example 131: tert-butyl ((1S)-1-(4,4-difluorocyclohexyl)-2-((4-(2-methoxy-1-(4,4,4-trifluorobutanamido)ethyl)pyridin-2-yl)amino)-2-oxoethyl)carbamate (DS 1)

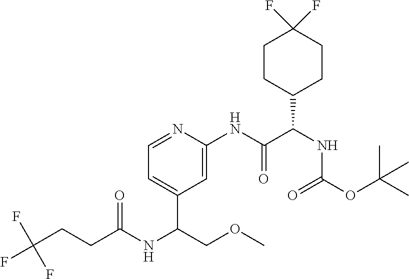 [SMILES: COCC(NC(=O)CCC(F)(F)F)c1ccnc(NC(=O)[C@@H](NC(=O)OC(C)(C)C)C2CCC(F)(F)CC2)c1]

The title compound was prepared using the procedure described for tert-butyl ((2S)-1,1-dicyclopropyl-3-((4-(cyclopropyl(4,4,4-trifluorobutanamido)methyl)pyridin-2-yl)amino)-3-oxopropan-2-yl)carbamate (Intermediate 34, step 6), employing 2-(tert-butoxycarbonylamino)-2-(4,4-difluorocyclohexyl)acetic acid as the amino acid and methoxymethyl Grignard reagent as in step Intermediate 34, step 2. The diastereomers were separated by chiral SFC (column: REGIS (S,S) Whelk-01 (250 mm×30 mm, 10 um); mobile phase: IPA (0.1% NH₃ (aq):ACN) to afford 780 mg of the title compound. LC/MS: m/z=567.4 [M+H]⁺; rt: 2.21 min (LC/MS-method A). ¹H NMR (400 MHz, DMSO-d₆) δ ppm: 10.43 (s, 1H), 8.67 (d, J=7.8 Hz, 1H), 8.25 (d, J=5.1 Hz, 1H), 8.05 (s, 1H), 7.07 (bd, 1H), 7.06 (dd, J=5.1 Hz, 1.3 Hz, 1H), 4.96 (m, 1H), 4.19 (bdd, 1H), 3.57-3.48 (m, 2H), 3.25 (s, 3H), 2.48-2.43 (m, 4H), 2.01 (m, 2H), 1.82-1.59 (m, 5H), 1.44-1.30 (m, 2H), 1.38 (s, 9H).

### Example 132: tert-butyl ((1S)-1-(4,4-difluorocyclohexyl)-2-((4-(2-methoxy-1-(4,4,4-trifluorobutanamido)ethyl)pyridin-2-yl)amino)-2-oxoethyl)carbamate (DS 2)

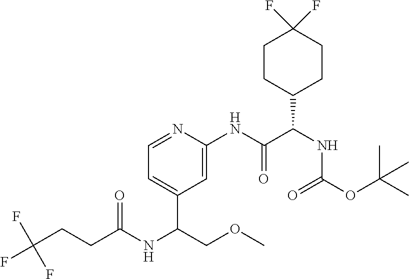 [SMILES: COCC(NC(=O)CCC(F)(F)F)c1ccnc(NC(=O)[C@@H](NC(=O)OC(C)(C)C)C2CCC(F)(F)CC2)c1]

840 mg of the title compound was obtained as diastereomer 2 from the chiral SFC separation in Example 131. LC/MS: m/z=567.4 [M+H]⁺; rt: 2.21 min (LC/MS-method A). ¹H NMR (400 MHz, DMSO-d₆) δ ppm: 10.40 (s, 1H), 8.67 (d, J=7.8 Hz, 1H), 8.25 (d, J=5.1 Hz, 1H), 8.05 (s, 1H), 7.07 (bd, 1H), 7.06 (dd, J=5.1 Hz, 1.3 Hz, 1H), 4.96 (m, 1H), 4.19 (bdd, 1H), 3.57-3.48 (m, 2H), 3.25 (s, 3H), 2.48-2.43 (m, 4H), 2.01 (m, 2H), 1.82-1.59 (m, 5H), 1.44-1.30 (m, 2H), 1.38 (s, 9H).

### Example 133: 4-cyclopropyl-N-((1S)-1-(4,4-difluorocyclohexyl)-2-((4-(2-methoxy-1-(4,4,4-trifluorobutanamido)ethyl)pyridin-2-yl)amino)-2-oxoethyl)-1,2,5-oxadiazole-3-carboxamide (DS 1)

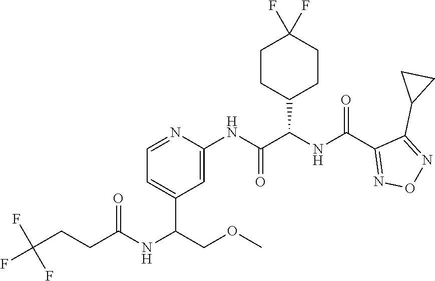 [SMILES: COCC(NC(=O)CCC(F)(F)F)c1ccnc(NC(=O)[C@@H](NC(=O)c2nonc2C2CC2)C2CCC(F)(F)CC2)c1]

Following the procedure described for Example 39, using 28 mg N-(1-(2-((S)-2-amino-2-(4,4-difluorocyclohexyl)acetamido)pyridin-4-yl)-2-methoxyethyl)-4,4,4-trifluoro-butanamide diastereomer 1 (Intermediate 45 DS1) and 10 mg 4-cyclopropyl-1,2,5-oxadiazole-3-carboxylic acid, 25 mg of the title compound was obtained. LC/MS: m/z=603.3 [M+H]⁺; rt: 2.29 min (LC/MS-method A). ¹H NMR (400 MHz, DMSO-de) δ ppm: 10.83 (s, 1H), 9.30 (d, J=8.0 Hz, 1H), 8.69 (d, J=8.0 Hz, 1H), 8.28 (d, J=5.2 Hz, 1H), 8.05 (s, 1H), 7.11 (dd, J=5.2 Hz, 1.3 Hz, 1H), 4.98 (m, 1H), 4.73 (dd, J=8.0 Hz, 8.0 Hz, 1H), 3.55-3.49 (m, 2H), 3.25 (s, 3H), 2.48-2.43 (m, 4H), 2.28 (m, 1H), 2.10-2.00 (m, 3H), 1.90-1.69 (m, 4H), 1.49 (m, 1H), 1.37 (m, 1H), 1.13 (m, 2H), 0.98 (m, 2H).

### Example 134: 4-cyclopropyl-N-((1S)-1-(4,4-difluorocyclohexyl)-2-((4-(2-methoxy-1-(4,4,4-trifluorobutanamido)ethyl)pyridin-2-yl)amino)-2-oxoethyl)-1,2,5-oxadiazole-3-carboxamide (DS 2)

Following the procedure described for Example 39, using 28 mg N-(1-(2-((S)-2-amino-2-(4,4-difluorocyclohexyl)acetamido)pyridin-4-yl)-2-methoxyethyl)-4,4,4-trifluoro-butanamide diastereomer 1 (Intermediate 45 DS2) and 10 mg 4-cyclopropyl-1,2,5-oxadiazole-3-carboxylic acid, 26 mg of the title compound was obtained. LC/MS: m/z=603.3 [M+H]⁺; rt: 2.29 min (LC/MS-method A). ¹H NMR (400 MHz, DMSO-d₆) δ ppm: 10.84 (s, 1H), 9.31 (d, J=8.0 Hz, 1H), 8.69 (d, J=8.0 Hz, 1H), 8.28 (d, J=5.2 Hz, 1H), 8.04 (s, 1H), 7.12 (dd, J=5.2 Hz, 1.3 Hz, 1H), 4.98 (m, 1H), 4.72 (dd, J=8.0 Hz, 8.0 Hz, 1H), 3.55-3.49 (m, 2H), 3.25 (s, 3H), 2.48-2.43 (m, 4H), 2.28 (m, 1H), 2.10-2.00 (m, 3H), 1.90-1.69 (m, 4H), 1.49 (m, 1H), 1.37 (m, 1H), 1.13 (m, 2H), 0.98 (m, 2H).

### Example 135: N-((1S)-2-((4-(2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)-5-methylpyrimidine-4-carboxamide (DS2)

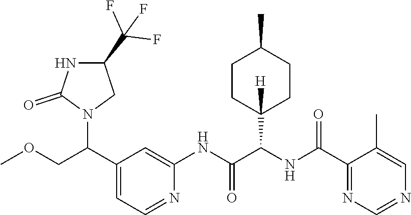

2-Chloro-N-((1S)-2-((4-(2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)-5-methyl-pyrimidine-4-carboxamide (DS2) (Example 095, 22 mg) was dissolved in dry methanol (3.0 ml) under Ar. After adding a small amount of Raney-Nickel in water with a spatula, a H₂-ballon was connected to the flask, and after stirring for 5.5 h, the reaction mixture stood overnight. Then the Raney-Nickel was filtered off, and the filtrate was concentrated in vacuo. The residue was purified by preparative RP HPLC (Purosphere® STAR-RP18, 25 mm×250 mm, 10 μm; 30 ml/min, from 95% H₂O/5% ACN to 10% H₂O/90% ACN in 45 min). The pure product containing fractions were combined, the ACN was partly removed, and the residue freeze dried to yield 7 mg of the title compound. LC/MS: m/z=578.4 [M+H]⁺; rt: 2.13 min (LC/MS-method A). ¹H NMR (400 MHz, DMSO-de) δ ppm: 10.74 (s, 1 H), 9.13 (s, 1H), 8.84 (s, 1H), 8.81 (d, J=8.7 Hz, 1H), 8.30 (d, J=5.2 Hz, 1H), 8.01 (s, 1H), 7.63 (bd, 1H), 7.00 (dd, J=5.2 Hz, 1.3 Hz, 1H), 5.01 (dd, J=8.1 Hz, 5.1 Hz, 1H), 4.65 (dd, J=8.7 Hz, 6.9 Hz, 1H), 4.44 (m, 1H), 3.83 (dd, J=10.5 Hz, 8.1 Hz, 1H), 3.77-3.72 (m, 2H), ~3.30 (s, 3H, signal below water signal), 3.29 (dd, 1H, signal below water signal), 2.46 (s, 3H), 1.82-1.62 (m, 5H), 1.28-1.17 (m, 2H), 1.07 (m, 1H), 0.91-0.82 (m 2H), 0.84 (d, J=6.5 Hz, 3H).

### Example 136: N-((1S)-1-(4,4-difluorocyclohexyl)-2-((4-(2-methoxy-1-(4,4,4-trifluoro-butanamido)ethyl)pyridin-2-yl)amino)-2-oxoethyl)-1-methyl-1H-pyrazole-5-carboxamide (DS 1)

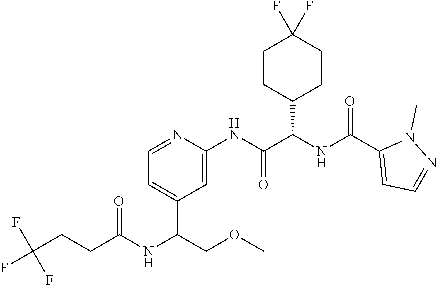 [SMILES: COCC(NC(=O)CCC(F)(F)F)c1ccnc(NC(=O)[C@@H](NC(=O)c2ccnn2C)C2CCC(F)(F)CC2)c1]

Following the procedure described for Example 33, using 33 mg N-(1-(2-((S)-2-amino-2-(4,4-difluorocyclohexyl)acetamido)pyridin-4-yl)-2-methoxyethyl)-4,4,4-trifluoro-butanamide diastereomer 1 (Intermediate 45 DS1) and 10 mg 2-methylpyrazole-3-carboxylic acid, 39 mg of the title compound was obtained. LC/MS: m/z=575.3 [M+H]⁺; rt: 1.88 min (LC/MS-method A). ¹H NMR (400 MHz, DMSO-d₆) δ ppm: 10.91 (s, 1H), 8.71 (d, J=7.8 Hz, 1H), 8.62 (d, J=7.8 Hz, 1H), 8.28 (d, J=5.2 Hz, 1H), 8.02 (s, 1H), 7.47 (d, J=2.0 Hz, 1H), 7.14 (dd, J=5.2 Hz, 1.3 Hz, 1H), 7.04 (d, J=2.0 Hz, 1H), 4.98 (m, 1H), 4.61 (dd, J=8.8 Hz, 7.8 Hz, 1H), 4.02 (s, 3H), 3.58-3.49 (m, 2H), 3.25 (s, 3H), 2.48-2.43 (m, 4H), 2.09-1.67 (m, 7H), 1.49 (m, 1H), 1.35 (m, 1H).

### Example 137: N-((1S)-1-(4,4-difluorocyclohexyl)-2-((4-(2-methoxy-1-(4,4,4-trifluoro-butanamido)ethyl)pyridin-2-yl)amino)-2-oxoethyl)-1-isopropyl-1H-pyrazole-5-carboxamide (DS 1)

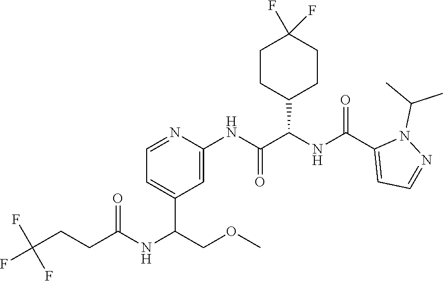 [SMILES: COCC(NC(=O)CCC(F)(F)F)c1ccnc(NC(=O)[C@@H](NC(=O)c2ccnn2C(C)C)C2CCC(F)(F)CC2)c1]

Following the procedure described for Example 33, using 33 mg N-(1-(2-((S)-2-amino-2-(4,4-difluorocyclohexyl)acetamido)pyridin-4-yl)-2-methoxyethyl)-4,4,4-trifluorobutanamide diastereomer 1 (Intermediate 45 DS1) and 12 mg 2-isopropylpyrazole-3-carboxylic acid, 40 mg of the title compound was obtained. LC/MS: m/z=603.4 [M+H]⁺; rt: 2.10 min (LC/MS-method A). ¹H NMR (400 MHz, DMSO-de) δ ppm: 10.86 (s, 1H), 8.71 (d, J=7.8 Hz, 1H), 8.60 (d, J=7.8 Hz, 1H), 8.28 (d, J=5.2 Hz, 1H), 8.03 (s, 1H), 7.51 (d, J=2.0 Hz, 1H), 7.13 (dd, J=5.2 Hz, 1.3 Hz, 1H), 6.94 (d, J=2.0 Hz, 1H), 5.36 (m, 1H), 4.98 (m, 1H), 4.60 (dd, J=8.7 Hz, 7.8 Hz, 1H), 3.58-3.49 (m, 2H), 3.25 (s, 3H), 2.48-2.43 (m, 4H), 2.09-1.67 (m, 7H), 1.49 (m, 1H), 1.36 (d, J=6.7 Hz, 3H), 1.35 (m, 1H), 1.34 (d, J=6.7 Hz, 3H).

### Example 138: N-((1S)-1-(4,4-difluorocyclohexyl)-2-((4-(2-methoxy-1-(4,4,4-trifluoro-butanamido)ethyl)pyridin-2-yl)amino)-2-oxoethyl)-1-ethyl-1H-pyrazole-5-carboxamide (DS 1)

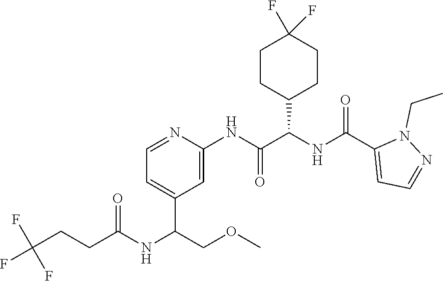 [SMILES: CCn1nccc1C(=O)N[C@H](C(=O)Nc1cc(C(COC)NC(=O)CCC(F)(F)F)ccn1)C1CCC(F)(F)CC1]

Following the procedure described for Example 33, using 33 mg N-(1-(2-((S)-2-amino-2-(4,4-difluorocyclohexyl)acetamido)pyridin-4-yl)-2-methoxyethyl)-4,4,4-trifluorobutanamide diastereomer 1 (Intermediate 45 DS1) and 11 mg 2-ethylpyrazole-3-carboxylic acid, 38 mg of the title compound was obtained. LC/MS: m/z=589.3 [M+H]⁺; rt: 1.99 min (LC/MS-method A). ¹H NMR (400 MHz, DMSO-d₆) δ ppm: 10.91 (s, 1H), 8.71 (d, J=7.8 Hz, 1H), 8.62 (d, J=7.8 Hz, 1H), 8.28 (d, J=5.2 Hz, 1H), 8.02 (s, 1H), 7.49 (d, J=2.0 Hz, 1H), 7.14 (dd, J=5.2 Hz, 1.3 Hz, 1H), 7.01 (d, J=2.0 Hz, 1H), 4.98 (m, 1H), 4.61 (dd, J=8.8 Hz, 7.8 Hz, 1H), 4.45 (q, J=7.2 Hz, 2H), 3.58-3.49 (m, 2H), 3.25 (s, 3H), 2.48-2.43 (m, 4H), 2.09-1.67 (m, 7H), 1.49 (m, 1H), 1.35 (m, 1H), 1.27 (t, J=7.2 Hz, 3H).

### Example 139: 1-cyclopropyl-N-((1S)-1-(4,4-difluorocyclohexyl)-2-((4-(2-methoxy-1-(4,4,4-trifluorobutanamido)ethyl)pyridin-2-yl)amino)-2-oxoethyl)-1H-pyrazole-5-carboxamide (DS 1)

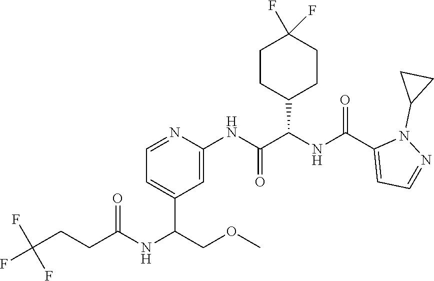 [SMILES: COCC(NC(=O)CCC(F)(F)F)c1ccnc(NC(=O)[C@@H](NC(=O)c2ccnn2C2CC2)C2CCC(F)(F)CC2)c1]

Following the procedure described for Example 33, using 33 mg N-(1-(2-((S)-2-amino-2-(4,4-difluorocyclohexyl)acetamido)pyridin-4-yl)-2-methoxyethyl)-4,4,4-trifluorobutanamide diastereomer 1 (Intermediate 45 DS1) and 12 mg 2-cyclopropylpyrazole-3-carboxylic acid, 40 mg of the title compound was obtained. LC/MS: m/z=601.4 [M+H]⁺; rt: 2.00 min (LC/MS-method A). ¹H NMR (400 MHz, DMSO-d₆) δ ppm: 10.91 (s, 1H), 8.71 (d, J=7.8 Hz, 1H), 8.63 (d, J=7.8 Hz, 1H), 8.28 (d, J=5.2 Hz, 1H), 8.03 (s, 1H), 7.42 (d, J=2.0 Hz, 1H), 7.14 (dd, J=5.2 Hz, 1.3 Hz, 1H), 6.95 (d, J=2.0 Hz, 1H), 4.98 (m, 1H), 4.64 (dd, J=8.7 Hz, 7.8 Hz, 1H), 4.38 (m, 1H), 3.58-3.49 (m, 2H), 3.25 (s, 3H), 2.48-2.43 (m, 4H), 2.09-1.67 (m, 7H), 1.50 (m, 1H), 1.36 (m, 1H), 1.05 (m, 2H), 0.91 (m, 2H).

### Example 140: N-((1S)-1-(4,4-difluorocyclohexyl)-2-((4-(2-methoxy-1-(4,4,4-trifluoro-butanamido)ethyl)pyridin-2-yl)amino)-2-oxoethyl)-1-methyl-1H-imidazole-2-carboxamide (DS 1)

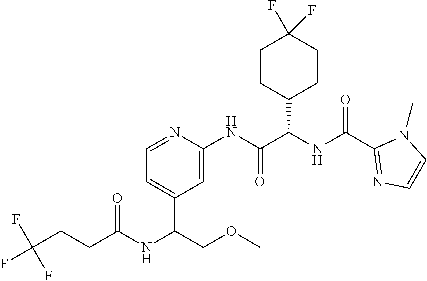 [SMILES: COCC(NC(=O)CCC(F)(F)F)c1ccnc(NC(=O)[C@@H](NC(=O)c2nccn2C)C2CCC(F)(F)CC2)c1]

Following the procedure described for Example 33, using 33 mg N-(1-(2-((S)-2-amino-2-(4,4-difluorocyclohexyl)acetamido)pyridin-4-yl)-2-methoxyethyl)-4,4,4-trifluoro-butanamide diastereomer 1 (Intermediate 45 DS1) and 10 mg 1-methylimidazole-2-carboxylic acid, 38 mg of the title compound was obtained. LC/MS: m/z=575.3 [M+H]⁺; rt: 1.88 min (LC/MS-method A). ¹H NMR (400 MHz, DMSO-d₆) δ ppm: 10.93 (s, 1H), 8.76 (bd, 1H), 8.71 (d, J=7.8 Hz, 1H), 8.28 (d, J=5.2 Hz, 1H), 8.03 (s, 1H), 7.58 (bs, 1H), 7.34 (bs, 1H), 7.13 (dd, J=5.2 Hz, 1.3 Hz, 1H), 4.98 (m, 1H), 4.71 (dd, 1H), 3.96 (s, 3H), 3.57-3.48 (m, 2H), 3.25 (s, 3H), 2.48-2.43 (m, 4H), 2.09-2.00 (m, 3H), 1.89-1.71 (m, 4H), 1.46 (m, 1H), 1.34 (m, 1H).

### Example 141: N-((1S)-1-(4,4-difluorocyclohexyl)-2-((4-(2-methoxy-1-(4,4,4-trifluoro-butanamido)ethyl)pyridin-2-yl)amino)-2-oxoethyl)-1-isopropyl-1H-imidazole-2-carboxamide (DS 1)

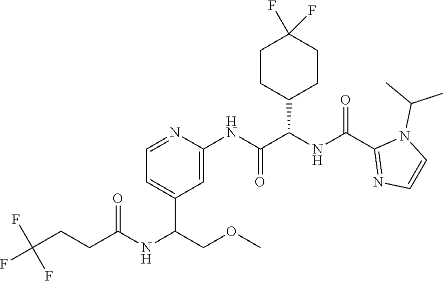 [SMILES: COCC(NC(=O)CCC(F)(F)F)c1ccnc(NC(=O)[C@@H](NC(=O)c2nccn2C(C)C)C2CCC(F)(F)CC2)c1]

Following the procedure described for Example 33, using 33 mg N-(1-(2-((S)-2-amino-2-(4,4-difluorocyclohexyl)acetamido)pyridin-4-yl)-2-methoxyethyl)-4,4,4-trifluorobutanamide diastereomer 1 (Intermediate 45 DS1) and 16 mg 1-isopropylimidazole-2-carboxylic acid, 30 mg of the title compound was obtained. LC/MS: m/z=603.3 [M+H]⁺; rt: 2.16 min (LC/MS-method A). ¹H NMR (400 MHz, DMSO-d₆) δ ppm: 10.96 (s, 1H), 8.90 (bd, 1H), 8.71 (d, J=7.8 Hz, 1H), 8.28 (d, J=5.2 Hz, 1H), 8.03 (s, 1H), 7.84 (bs, 1H), 7.44 (bs, 1H), 7.13 (dd, J=5.2 Hz, 1.3 Hz, 1H), 5.37 (m, 1H), 4.98 (m, 1H), 4.72 (dd, 1H), 3.58-3.49 (m, 2H), 3.25 (s, 3H), 2.48-2.43 (m, 4H), 2.09-2.00 (m, 3H), 1.90-1.71 (m, 4H), 1.47 (m, 1H), 1.42 (d, J=6.6 Hz, 3H), 1.40 (d, J=6.6 Hz, 3H), 1.35 (m, 1H).

### Example 142: 1-(cyclopropylmethyl)-N-((1S)-1-(4,4-difluorocyclohexyl)-2-((4-(2-methoxy-1-(4,4,4-trifluorobutanamido)ethyl)pyridin-2-yl)amino)-2-oxoethyl)-1H-pyrazole-5-carboxamide (DS 1)

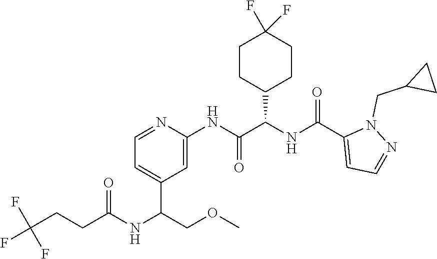 [SMILES: COCC(NC(=O)CCC(F)(F)F)c1ccnc(NC(=O)[C@@H](NC(=O)c2ccnn2CC2CC2)C2CCC(F)(F)CC2)c1]

Following the procedure described for Example 33, using 33 mg N-(1-(2-((S)-2-amino-2-(4,4-difluorocyclohexyl)acetamido)pyridin-4-yl)-2-methoxyethyl)-4,4,4-trifluorobutanamide diastereomer 1 (Intermediate 45 DS1) and 13 mg 2-(cyclopropylmethyl)pyrazole-2-carboxylic acid, 43 mg of the title compound was obtained. LC/MS: m/z=615.4 [M+H]⁺; rt: 2.12 min (LC/MS-method A). ¹H NMR (400 MHz, DMSO-de) δ ppm: 10.89 (s, 1H), 8.71 (d, J=7.8 Hz, 1H), 8.66 (d, J=7.9 Hz, 1H), 8.28 (d, J=5.2 Hz, 1H), 8.02 (s, 1H), 7.50 (d, J=2.0 Hz, 1H), 7.13 (dd, J=5.2 Hz, 1.3 Hz, 1H), 7.00 (d, J=2.0 Hz, 1H), 4.98 (m, 1H), 4.61 (dd, J=8.7 Hz, 7.9 Hz, 1H), 4.31 (d, J=7.1 Hz, 2H), 3.58-3.49 (m, 2H), 3.25 (s, 3H), 2.48-2.43 (m, 4H), 2.09-1.67 (m, 7H), 1.49 (m, 1H), 1.35 (m, 1H), 1.20 (m, 1H), 0.38 (m, 2H), 0.29 (m, 2H).

### Example 143: N-((1S)-1-(4,4-difluorocyclohexyl)-2-((4-(2-methoxy-1-(4,4,4-trifluoro-butanamido)ethyl)pyridin-2-yl)amino)-2-oxoethyl)-1-methyl-1H-pyrazole-5-carboxamide (DS 2)

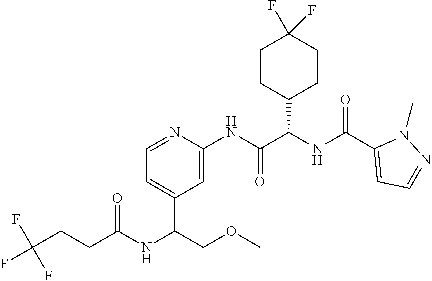 [SMILES: COCC(NC(=O)CCC(F)(F)F)c1ccnc(NC(=O)[C@@H](NC(=O)c2ccnn2C)C2CCC(F)(F)CC2)c1]

Following the procedure described for Example 33, using 36 mg of N-(1-(2-((S)-2-amino-2-(4,4-difluorocyclohexyl)acetamido)pyridin-4-yl)-2-methoxyethyl)-4,4,4-trifluoro-butanamide diastereomer 1 (Intermediate 45 DS2) and 11 mg 1-methylimidazole-2-carboxylic acid, 43 mg of the title compound was obtained. LC/MS: m/z=575.3 [M+H]⁺; rt: 1.88 min (LC/MS-method A). ¹H NMR (400 MHz, DMSO-d₆) δ ppm: 10.80 (s, 1H), 8.69 (d, J=7.8 Hz, 1H), 8.59 (d, J=8.0 Hz, 1H), 8.27 (d, J=5.2 Hz, 1H), 8.03 (s, 1H), 7.47 (d, J=2.0 Hz, 1H), 7.12 (dd, J=5.2 Hz, 1.3 Hz, 1H), 7.04 (d, J=2.0 Hz, 1H), 4.98 (m, 1H), 4.61 (dd, J=8.7 Hz, 8.0 Hz, 1H), 4.07 (s, 3H), ~3.58-3.48 (m, 2H), 3.25 (s, 3H), 2.48-2.43 (m, 4H), 2.09-1.67 (m, 7H), 1.49 (m, 1H), 1.35 (m, 1H).

### Example 144: N-(1S)-1-(4,4-difluorocyclohexyl)-2-((4-(2-methoxy-1-(4,4,4-trifluorobutanamido)ethyl)pyridin-2-yl)amino)-2-oxoethyl)-1-ethyl-1H-pyrazole-5-carboxamide (DS 2)

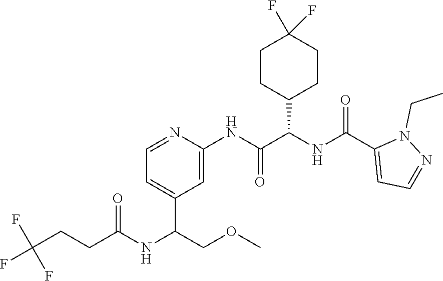 [SMILES: CCn1nccc1C(=O)N[C@H](C(=O)Nc1cc(C(COC)NC(=O)CCC(F)(F)F)ccn1)C1CCC(F)(F)CC1]

Following the procedure described for Example 33, using 36 mg N-(1-(2-((S)-2-amino-2-(4,4-difluorocyclohexyl)acetamido)pyridin-4-yl)-2-methoxyethyl)-4,4,4-trifluoro-butanamide diastereomer 1 (Intermediate 45 DS2) and 12 mg 2-ethylylpyrazole-3-carboxylic acid, 38 mg of the title compound was obtained. LC/MS: m/z=589.3 [M+H]⁺; rt: 1.98 min (LC/MS-method A). ¹H NMR (400 MHz, DMSO-d₆) δ ppm: 10.79 (s, 1H), 8.69 (d, J=7.8 Hz, 1H), 8.60 (d, J=7.9 Hz, 1H), 8.27 (d, J=5.2 Hz, 1H), 8.03 (s, 1H), 7.49 (d, J=2.0 Hz, 1H), 7.12 (dd, J=5.2 Hz, 1.3 Hz, 1H), 7.00 (d, J=2.0 Hz, 1H), 4.98 (m, 1H), 4.61 (dd, J=8.6 Hz, 7.9 Hz, 1H), 4.46 (m, 2H), 3.58-3.48 (m, 2H), 3.25 (s, 3H), 2.48-2.43 (m, 4H), 2.09-1.67 (m, 7H), 1.48 (m, 1H), 1.35 (m, 1H), 1.27 (t, J=7.1 Hz, 3H).

### Example 145: N-(1S)-1-(4,4-difluorocyclohexyl)-2-((4-(2-methoxy-1-(4,4,4-trifluoro-butanamido)ethyl)pyridin-2-yl)amino)-2-oxoethyl)-1-isopropyl-1H-pyrazole-5-carboxamide (DS 2)

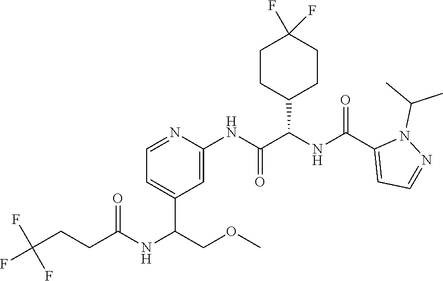 [SMILES: COCC(NC(=O)CCC(F)(F)F)c1ccnc(NC(=O)[C@@H](NC(=O)c2ccnn2C(C)C)C2CCC(F)(F)CC2)c1]

Following the procedure described Example 33, using 36 mg N-(1-(2-((S)-2-amino-2-(4,4-difluorocyclohexyl)acetamido)pyridin-4-yl)-2-methoxyethyl)-4,4,4-trifluoro-butanamide diastereomer 1 (Intermediate 45 DS2) and 13 mg 2-isopropylpyrazole-3-carboxylic acid, 39 mg of the title compound was obtained. LC/MS: m/z=603.4 [M+H]⁺; rt: 2.11 min (LC/MS-method A). ¹H NMR (400 MHz, DMSO-de) δ ppm: 10.84 (s, 1H), 8.70 (d, J=7.8 Hz, 1H), 8.60 (d, J=7.8 Hz, 1H), 8.28 (d, J=5.2 Hz, 1H), 8.02 (s, 1H), 7.51 (d, J=2.0 Hz, 1H), 7.13 (dd, J=5.2 Hz, 1.3 Hz, 1H), 6.94 (d, J=2.0 Hz, 1H), 5.36 (m, 1H), 4.98 (m, 1H), 4.59 (dd, J=8.6 Hz, 7.8 Hz, 1H), 3.58-3.49 (m, 2H), 3.25 (s, 3H), 2.48-2.43 (m, 4H), 2.09-1.67 (m, 7H), 1.48 (m, 1H), 1.36 (d, J=6.6 Hz, 3H), 1.35 (m, 1H), 1.34 (d, J=6.6 Hz, 3H).

### Example 146: 1-cyclopropyl-N-((1S)-1-(4,4-difluorocyclohexyl)-2-((4-(2-methoxy-1-(4,4,4-trifluorobutanamido)ethyl)pyridin-2-yl)amino)-2-oxoethyl)-1H-pyrazole-5-carboxamide (DS 2)

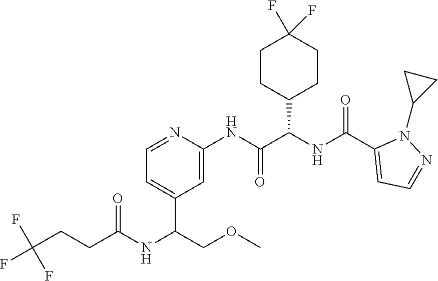 [SMILES: COCC(NC(=O)CCC(F)(F)F)c1ccnc(NC(=O)[C@@H](NC(=O)c2ccnn2C2CC2)C2CCC(F)(F)CC2)c1]

Following the procedure described for Example 33, using 36 mg N-(1-(2-((S)-2-amino-2-(4,4-difluorocyclohexyl)acetamido)pyridin-4-yl)-2-methoxyethyl)-4,4,4-trifluoro-butanamide diastereomer 1 (Intermediate 45 DS2) and 13 mg 2-cyclopropylpyrazole-3-carboxylic acid, 40 mg of the title compound was obtained. LC/MS: m/z=601.4 [M+H]⁺; rt: 2.00 min (LC/MS-method A). ¹H NMR (400 MHz, DMSO-d₆) δ ppm: 10.84 (s, 1H), 8.70 (d, J=7.8 Hz, 1H), 8.62 (d, J=7.9 Hz, 1H), 8.27 (d, J=5.2 Hz, 1H), 8.03 (s, 1H), 7.43 (d, J=2.0 Hz, 1H), 7.13 (dd, J=5.2 Hz, 1.3 Hz, 1H), 6.95 (d, J=2.0 Hz, 1H), 4.98 (m, 1H), 4.63 (dd, J=8.6 Hz, 7.9 Hz, 1H), 4.39 (m, 1H), ~3.58-3.48 (m, 2H, signals below water signal), 3.25 (s, 3H), 2.48-2.43 (m, 4H), 2.09-1.68 (m, 7H), 1.50 (m, 1H), 1.37 (m, 1H), 1.06 (m, 2H), 0.92 (m, 2H).

### Example 147: 1-(cyclopropylmethyl)-N-((1S)-1-(4,4-difluorocyclohexyl)-2-((4-(2-methoxy-1-(4,4,4-trifluorobutanamido)ethyl)pyridin-2-yl)amino)-2-oxoethyl)-1H-pyrazole-5-carboxamide (DS 2)

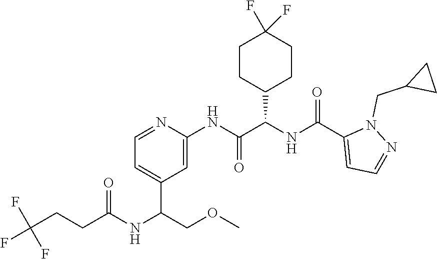 [SMILES: COCC(NC(=O)CCC(F)(F)F)c1ccnc(NC(=O)[C@@H](NC(=O)c2ccnn2CC2CC2)C2CCC(F)(F)CC2)c1]

Following the procedure described for Example 33, using 36 mg N-(1-(2-((S)-2-amino-2-(4,4-difluorocyclohexyl)acetamido)pyridin-4-yl)-2-methoxyethyl)-4,4,4-trifluoro-butanamide diastereomer 1 (Intermediate 45 DS2) and 14 mg 2-(cyclopropylmethyl)pyrazole-2-carboxylic acid, 38 mg of the title compound was obtained. LC/MS: m/z=615.4 [M+H]⁺; rt: 2.12 min (LC/MS-method A). ¹H NMR (400 MHz, DMSO-de) δ ppm: 10.82 (s, 1H), 8.69 (d, J=7.8 Hz, 1H), 8.64 (d, J=7.9 Hz, 1H), 8.27 (d, J=5.2 Hz, 1H), 8.02 (s, 1H), 7.50 (d, J=2.0 Hz, 1H), 7.13 (dd, J=5.2 Hz, 1.3 Hz, 1H), 7.00 (d, J=2.0 Hz, 1H), 4.98 (m, 1H), 4.61 (dd, J=8.8 Hz, 7.9 Hz, 1H), 4.31 (m, 2H), 3.57-3.49 (m, 2H), 3.25 (s, 3H), 2.48-2.43 (m, 4H), 2.09-1.68 (m, 7H), 1.48 (m, 1H), 1.35 (m, 1H), 1.20 (m, 1H), 0.38 (m, 2H), 0.29 (m, 2H).

### Example 148: N-((1S)-1-(4,4-difluorocyclohexyl)-2-((4-(2-methoxy-1-(4,4,4-trifluoro-butanamido)ethyl)pyridin-2-yl)amino)-2-oxoethyl)-1-isopropyl-1H-imidazole-2-carboxamide (DS 2)

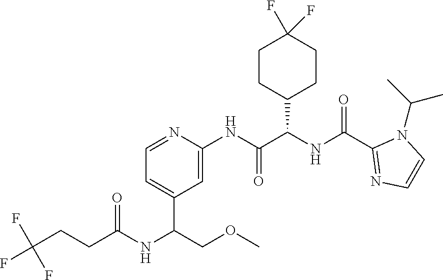 [SMILES: COCC(NC(=O)CCC(F)(F)F)c1ccnc(NC(=O)[C@@H](NC(=O)c2nccn2C(C)C)C2CCC(F)(F)CC2)c1]

Following the procedure described for Example 33, using 33 mg N-(1-(2-((S)-2-amino-2-(4,4-difluorocyclohexyl)acetamido)pyridin-4-yl)-2-methoxyethyl)-4,4,4-trifluoro-butanamide diastereomer 1 (Intermediate 45 DS2) and 18 mg 1-isopropylimidazole-2-carboxylic acid, 28 mg of the title compound was obtained. LC/MS: m/z=603.3 [M+H]⁺; rt: 2.13 min (LC/MS-method A). ¹H NMR (400 MHz, DMSO-d₆) δ ppm: 10.92 (s, 1H), 8.81 (bd, J=7.9 Hz, 1H), 8.70 (d, J=7.7 Hz, 1H), 8.28 (d, J=5.2 Hz, 1H), 8.03 (s, 1H), 7.81 (d, J=0.8 Hz, 1H), 7.38 (bs, 1H), 7.12 (dd, J=5.2 Hz, 1.3 Hz, 1H), 5.39 (m, 1H), 4.97 (m, 1H), 4.71 (dd, J=7.9 Hz, 7.8 Hz, 1H), 3.57-3.49 (m, 2H), 3.25 (s, 3H), 2.48-2.43 (m, 4H), 2.09-2.00 (m, 3H), 1.88-1.72 (m, 4H), 1.46 (m, 1H), 1.42 (d, J=6.6 Hz, 3H), 1.40 (d, J=6.6 Hz, 3H), 1.34 (m, 1H).

Using (2S)-2-amino-N-(4-((R or S)-2-methoxy-1-((S)-2-oxo-4-(trifluoromethyl)-imidazolidin-1-yl)ethyl)pyridin-2-yl)-2-((1r,4S)-4-methylcyclohexyl)acetamide hydrochloride salt (DS2) (Intermediate 18) as starting material analogously to Example 102, were prepared:

| Ex. | Acid | Structure | Name | Yield |
|---|---|---|---|---|
| 149 | | | 2,2-difluoro-N-((S)-2-((4- | 12 mg, |
| | | | ((S)-2-methoxy-1-((S)-2- | 52% |
| | | | oxo-4-(trifluoromethyl)- | |
| | | | imidazolidin-1-yl)ethyl)- | |
| | | | pyridin-2-yl)amino)-1- | |
| | | | ((1r,4S)-4-methylcyclo- | |
| | | | hexyl)-2-oxoethyl)-2-(p- | |
| | | | tolyl)acet-amide (DS2) | |
| 150 | | | 2,2-difluoro-N-((1S)-2-((4- | 12 mg, |
| | | | (2-methoxy-1-((S)-2-oxo- | 52% |
| | | | 4-(trifluoromethyl)- | |
| | | | imidazolidin-1-yl)ethyl)- | |
| | | | pyridin-2-yl)amino)-1- | |
| | | | ((1r,4S)-4-methylcyclo- | |
| | | | hexyl)-2-oxoethyl)-2- | |
| | | | phenylacetamide (DS2) | |

### Example 149

LC/MS: m/z=626.4 [M+H]⁺; rt: 2.56 min (LC/MS-method A). ¹H NMR (400 MHz, DMSO-d₆) δ ppm: 10.63 (s, 1H), 8.89 (d, J=8.1 Hz, 1H), 8.29 (d, J=5.2 Hz, 1H), 7.97 (bs, 1H), 7.63 (d, J=1.9 Hz, 1H), 7.49 (d, J=8.2 Hz, 2H), 7.31 (d, J=8.2 Hz, 2H), 7.00 (dd, J=5.2 Hz, 1.3 Hz, 1H), 5.01 (dd, J=8.1 Hz, 5.1 Hz, 1H), 4.44 (m, 1H), 4.37 (dd, J=8.6 Hz, 8.1 Hz, 1H), 3.83 (dd, J=10.5 Hz, 8.1 Hz, 1H), 3.77-3.72 (m, 2H), ~3.30 (s, 3H, signal below water signal), 3.29 (dd, 1H, signal below water signal), 2.34 (s, 3H), 1.84-1.49 (m 5H), 1.27-1.09 (m, 2H), 0.99-0.77 (m, 3H), 0.83 (d, J=6.5 Hz, 3H).

### Example 150

LC/MS: m/z=612.4 [M+H]⁺; rt: 2.45 min (LC/MS-method A). ¹H NMR (400 MHz, DMSO-de) δ ppm: 10.64 (s, 1H), 8.95 (d, J=8.0 Hz, 1H), 8.29 (d, J=5.2 Hz, 1H), 7.98 (bs, 1H), 7.63-7.60 (m, 3H), 7.57-7.49 (m, 3H), 7.00 (dd, J=5.2 Hz, 1.3 Hz, 1H), 5.01 (dd, J=8.1 Hz, 5.1 Hz, 1H), 4.44 (m, 1H), 4.38 (dd, J=8.8 Hz, 8.0 Hz, 1H), 3.83 (dd, J=10.5 Hz, 8.1 Hz, 1H), 3.77-3.72 (m, 2H), ~3.30 (s, 3H, signal below water signal), 3.29 (dd, 1H, signal below water signal), 1.80 (m, 1H), 1.70-1.62 (m 3H), 1.51 (m, 1H), 1.23 (m, 1H), 1.14 (m, 1H), 0.99-0.78 (m, 3H), 0.83 (d, J=6.5 Hz, 3H).

### Example 151: Tert-butyl ((1S)-1-((1r,4S)-4-methylcyclohexyl)-2-oxo-2-((4-(((S)-2-oxo-4-(trifluoromethyl)-imidazolidin-1-yl)methyl-d)pyridin-2-yl)amino)ethyl)carbamate

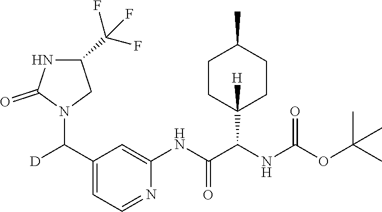

Following the procedure of Example 001, and using tert-butyl ((1S)-2-((4-((((S)-2-amino-3,3,3-trifluoropropyl)amino)methyl-d)pyridin-2-yl)amino)-1-((1 r,4S)-4-methylcyclo-hexyl)-2-oxoethyl)carbamate (Intermediate 44; 95 mg), 86 mg (86%) of the title compound was obtained. LC/MS: m/z=515.3 [M+H]⁺; rt: 2.33 min (LC/MS-method A). ¹H NMR (400 MHz, DMSO-d₆) δ ppm: 10.34 (s, 1H), 8.27 (d, J=5.1 Hz, 1H), 7.97 (s, 1H), 7.65 (d, J=1.9 Hz, 1H), 6.93 (dd, J=5.1 Hz, 1.3 Hz, 1H), 6.91 (d, signal overlap, 1H), 4.42 (m, 1H), 4.37 (bs, 0.45H), 4.20 (bs, 0.55H), 4.05 (dd, 1H), 3.58 (dd, =10.0 Hz, 10.0 Hz 1H), ~3.30 (dd, 1H, signal below water signal), 1.71-1.49 (m, 5H), 1.38 (s, 9H), 1.30-0.99 (m 3H), 0.87-0.77 (m 2H), 0.83 (d, J=6.5 Hz, 3H).

### Example 152: 4-cyclopropyl-N—((S)-1-((1r,4S)-4-methylcyclohexyl)-2-oxo-2-((4-(((3R,5S)-2-oxo-5-(trifluoromethyl)pyrrolidin-3-yl)methyl)pyridin-2-yl)amino)ethyl)-1,2,5-oxadiazole-3-carboxamide

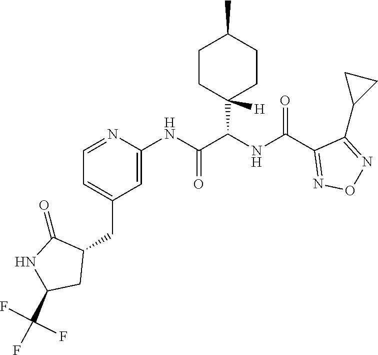 [SMILES: C[C@H]1CC[C@H]([C@H](NC(=O)c2nonc2C2CC2)C(=O)Nc2cc(C[C@@H]3C[C@@H](C(F)(F)F)NC3=O)ccn2)CC1]

Following the procedure described for Example 39, using 35 mg of (S)-2-amino-2-((1r,4S)-4-methylcyclohexyl)-N-(4-(((3R,5S)-2-oxo-5-(trifluoromethyl)pyrrolidin-3-yl)methyl)pyridin-2-yl)acetamide (Intermediate 46) and 14 mg 4-cyclopropyl-1,2,5-oxadiazole-3-carboxylic acid, 3.5 mg the title compound was obtained. LC/MS: m/z=549.3 [M+H]⁺; rt: 2.48 min (LC/MS-method A). ¹H NMR (400 MHz, DMSO-de) δ ppm: 10.74 (s, 1H), 9.17 (d, J=8.1 Hz, 1H), 8.67 (s, 1H), 8.24 (d, J=5.1 Hz, 1H), 7.95 (s, 1H, below DMF signal), 7.07 (dd, J=5.1 Hz, 1.3 Hz, 1H), 4.62 (dd, J=8.1 Hz, 7.7 Hz, 1H), 4.21 (m, 1H), 3.03 (dd, J=13.7 Hz, 4.1 Hz, 1H), 2.74 (m, 1H), 2.62 (dd, J=13.7 Hz, 9.7 Hz, 1H), 2.27 (m, 1H), 2.12-2.02 (m, 2H), 1.82-1.59 (m 5H), 1.29-1.20 (m 2H), 1.15-1.08 (m 3H), 0.98 (m, 2H), 0.91-0.82 (m 2H), 0.85 (d, J=6.5 Hz, 3H).

### Example 153: 1-ethyl-N—((S)-1-((1r,4S)-4-methylcyclohexyl)-2-oxo-2-((4-(((3R,5S)-2-oxo-5-(trifluoromethyl)pyrrolidin-3-yl)methyl)pyridin-2-yl)amino)ethyl)-1H-pyrazole-5-carboxamide

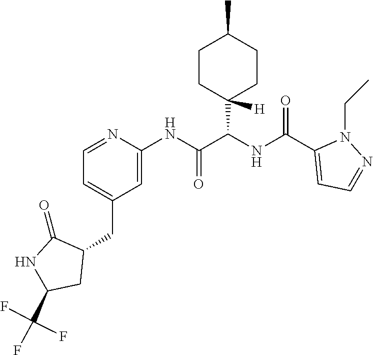 [SMILES: CCn1nccc1C(=O)N[C@H](C(=O)Nc1cc(C[C@@H]2C[C@@H](C(F)(F)F)NC2=O)ccn1)[C@H]1CC[C@H](C)CC1]

Following the procedure described for Example 33, using 35 mg of (S)-2-amino-2-((1r,4S)-4-methylcyclohexyl)-N-(4-(((3R,5S)-2-oxo-5-(trifluoromethyl)pyrrolidin-3-yl)methyl)pyridin-2-yl)acetamide (Intermediate 46) and 13 mg 2-ethylpyrazole-3-carboxylic acid, 15 mg of the title compound was obtained. LC/MS: m/z=535.3 [M+H]⁺; rt: 2.15 min (LC/MS-method A). ¹H NMR (400 MHz, DMSO-d₆) δ ppm: 10.86 (bs, 1H), 8.68 (s, 1H), 8.50 (d, J=8.0 Hz, 1H), 8.24 (d, J=5.2 Hz, 1H), 7.90 (s, 1H), 7.48 (d, J=2.0 Hz, 1H), 7.12 (bd, J=5.2 Hz, 1H), 7.01 (d, J=2.0 Hz, 1H), 4.49 (dd, 1H), 4.44 (q, J=7.1 Hz, 2H), 4.21 (m, 1H), 3.04 (dd, J=13.7 Hz, 4.2 Hz, 1H), 2.76 (m, 1H), 2.65 (dd, J=13.7 Hz, 9.6 Hz, 1H), 2.14-2.01 (m, 2H), 1.85-1.58 (m 5H), 1.31-1.19 (m 2H), 1.26 (t, J=7.1 Hz, 3H), 1.07 (m 1H), 0.91-0.82 (m 2H), 0.85 (d, J=6.5 Hz, 3H).

### Example 154: 1-isopropyl-N—((S)-1-((1r,4S)-4-methylcyclohexyl)-2-oxo-2-((4-(((3R,5S)-2-oxo-5-(trifluoromethyl)pyrrolidin-3-yl)methyl)pyridin-2-yl)amino)ethyl)-1H-pyrazole-5-carboxamide

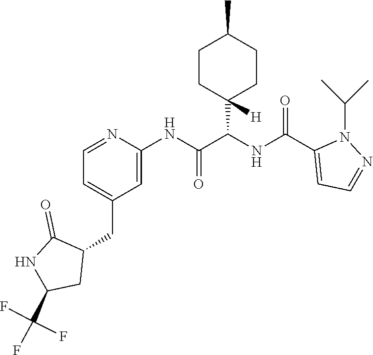 [SMILES: CC(C)n1nccc1C(=O)N[C@H](C(=O)Nc1cc(C[C@@H]2C[C@@H](C(F)(F)F)NC2=O)ccn1)[C@H]1CC[C@H](C)CC1]

Following the procedure described for Example 33, using 35 mg of (S)-2-amino-2-((1r,4S)-4-methylcyclohexyl)-N-(4-(((3R,5S)-2-oxo-5-(trifluoromethyl)pyrrolidin-3-yl)methyl)pyridin-2-yl)acetamide (Intermediate 46) and 14 mg 2-isopropylpyrazole-3-carboxylic acid, 14 mg of the title compound was obtained. LC/MS: m/z=549.4 [M+H]⁺; rt: 2.27 min (LC/MS-method A). ¹H NMR (400 MHz, DMSO-de) δ ppm: 10.86 (bs, 1H), 8.68 (s, 1H), 8.49 (d, J=7.9 Hz, 1H), 8.24 (d, J=5.3 Hz, 1H), 7.90 (s, 1H), 7.50 (d, J=2.0 Hz, 1H), 7.12 (bd, J=5.3 Hz, 1H), 6.95 (d, J=2.0 Hz, 1H), 5.37 (m, 1H), 4.48 (dd, J=8.3 Hz, 7.9 Hz, 1H), 4.21 (m, 1H), 3.04 (dd, J=13.7 Hz, 4.2 Hz, 1H), 2.76 (m, 1H), 2.65 (dd, J=13.7 Hz, 9.6 Hz, 1H), 2.14-2.01 (m, 2H), 1.85-1.58 (m 5H), 1.35 (d, J=6.7 Hz, 3H), 1.33 (d, J=6.7 Hz, 3H), 1.32-1.19 (m 2H), 1.08 (m 1H), 0.92-0.82 (m 2H), 0.85 (d, J=6.5 Hz, 3H).

### Example 155: 1-ethyl-N—((S)-1-((1r,4S)-4-methylcyclohexyl)-2-oxo-2-((4-(((3S,5S)-2-oxo-5-(trifluoromethyl)pyrrolidin-3-yl)methyl)pyridin-2-yl)amino)ethyl)-1H-pyrazole-5-carboxamide

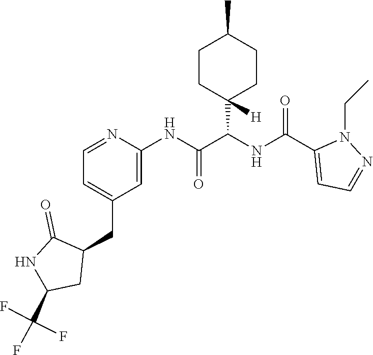 [SMILES: CCn1nccc1C(=O)N[C@H](C(=O)Nc1cc(C[C@H]2C[C@@H](C(F)(F)F)NC2=O)ccn1)[C@H]1CC[C@H](C)CC1]

Following the procedure described for Example 33, using 62 mg of (S)-2-amino-2-((1r,4S)-4-methylcyclohexyl)-N-(4-(((3S,5S)-2-oxo-5-(trifluoromethyl)pyrrolidin-3-yl)methyl)pyridin-2-yl)acetamide (Intermediate 47) and 21 mg 2-ethylpyrazole-3-carboxylic acid, 3.4 mg of the title compound was obtained. LC/MS: m/z=535.3 [M+H]⁺; rt: 2.13 min (LC/MS-method A). ¹H NMR (400 MHz, DMSO-d₆) δ ppm: 10.67 (s, 1H), 8.62 (s, 1H), 8.46 (d, J=8.0 Hz, 1H), 8.23 (d, J=5.2 Hz, 1H), 7.93 (s, 1H), 7.48 (d, J=2.0 Hz, 1H), 7.05 (bd, J=5.2 Hz, 1H), 7.00 (d, J=2.0 Hz, 1H), 4.49 (dd, 1H), 4.44 (q, J=7.1 Hz, 2H), 4.25 (m, 1H), 3.04 (dd, J=13.8 Hz, 4.3 Hz, 1H), 2.78 (m, 1H), 2.61 (dd, J=13.8 Hz, 10.0 Hz, 1H), 2.34 (m, 1H), 1.84-1.56 (m 6H), 1.31-1.20 (m 2H), 1.26 (t, J=7.1 Hz, 3H), 1.07 (m 1H), 0.91-0.82 (m 2H), 0.85 (d, J=6.5 Hz, 3H).

### Example 156: 1-isopropyl-N—((S)-1-((1r,4S)-4-methylcyclohexyl)-2-oxo-2-((4-(((3S,5S)-2-oxo-5-(trifluoromethyl)pyrrolidin-3-yl)methyl)pyridin-2-yl)amino)ethyl)-1H-pyrazole-5-carboxamide

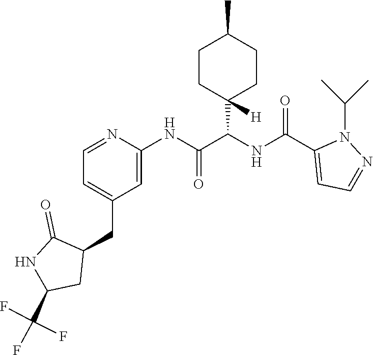 [SMILES: CC(C)n1nccc1C(=O)N[C@H](C(=O)Nc1cc(C[C@H]2C[C@@H](C(F)(F)F)NC2=O)ccn1)[C@H]1CC[C@H](C)CC1]

Following the procedure described for Example 33, using 62 mg of (S)-2-amino-2-((1r,4S)-4-methylcyclohexyl)-N-(4-(((3S,5S)-2-oxo-5-(trifluoromethyl)pyrrolidin-3-yl)methyl)pyridin-2-yl)acetamide (Intermediate 47) and 23 mg 2-isopropylpyrazole-3-carboxylic acid, 3.8 mg of the title compound was obtained. LC/MS: m/z=549.3 [M+H]⁺; rt: 2.26 min (LC/MS-method A). ¹H NMR (400 MHz, DMSO-d₆) δ ppm: 10.66 (s, 1H), 8.62 (s, 1H), 8.45 (d, J=8.0 Hz, 1H), 8.23 (d, J=5.2 Hz, 1H), 7.94 (s, 1H), 7.50 (d, J=2.0 Hz, 1H), 7.05 (dd, J=5.2 Hz, 1.2 Hz, 1H), 6.93 (d, J=2.0 Hz, 1H), 5.37 (m, 1H), 4.48 (dd, J=8.4 Hz, 8.0 Hz, 1H), 4.25 (m, 1H), 3.04 (dd, J=13.9 Hz, 4.3 Hz, 1H), 2.78 (m, 1H), 2.61 (dd, J=13.9 Hz, 9.9 Hz, 1H), 2.34 (m, 1H), 1.84-1.56 (m 6H), 1.35 (d, J=6.7 Hz, 3H), 1.33 (d, J=6.7 Hz, 3H), 1.32-1.22 (m 2H), 1.07 (m 1H), 0.91-0.82 (m 2H), 0.85 (d, J=6.5 Hz, 3H).

### Example 157: N-((1S)-1-(4,4-difluorocyclohexyl)-2-oxo-2-((4-(3,3,3-trifluoro-1-(4,4,4-trifluorobutanamido)propyl)pyridin-2-yl)amino)ethyl)-4-ethylisoxazole-5-carboxamide

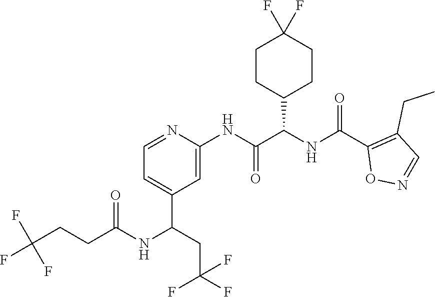 [SMILES: CCc1cnoc1C(=O)N[C@H](C(=O)Nc1cc(C(CC(F)(F)F)NC(=O)CCC(F)(F)F)ccn1)C1CCC(F)(F)CC1]

Following the procedure described for Example 39, using 27 mg N-(1-(2-((S)-2-amino-2-(4,4-difluorocyclohexyl)acetamido)pyridin-4-yl)-3,3,3-trifluoropropyl)-4,4,4-trifluoro-butanamide (Intermediate 57), 14 mg of the title compound was obtained. LC/MS: m/z=628.2 [M+H]⁺; rt: 2.35 min (LC/MS-method A). ¹H NMR (400 MHz, DMSO-d₆) δ ppm: 10.76 (s, 1H), 9.17 (d, J=8.1 Hz, 1H), 8.74 (d, J=7.7 Hz, 1H), 8.26 (d, J=5.2 Hz, 1H), 8.08 (br s 1H), 7.12 (dd, J=5.2, 1.5 Hz, 1H), 4.66-4.58 (m, 1H), 4.19-4.12 (m, 1H), 2.55-2.36 (m, 4H, overlapping solvent), 2.28 (tt, J=8.4, 5.0 Hz, 1H), 1.87-1.75 (m, 2H), 1.74-1.56 (m, 3H), 1.35-1.18 (m, 2H), 1.17-1.03 (m, 4H), 1.02-0.94 (m, 2H), 0.93-0.80 (m, 5H), 0.58-0.47 (m, 2H), 0.45-0.30 (m, 2H).

### Example 158: N—((S)-1-(4,4-difluorocyclohexyl)-2-oxo-2-((4-((R)-1-((S)-2-oxo-4-(trifluoromethyl)imidazolidin-1-yl)butyl)pyridin-2-yl)amino)ethyl)-4-ethylisoxazole-3-carboxamide

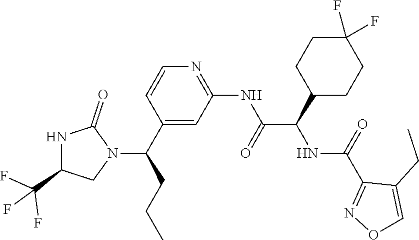 [SMILES: CCC[C@H](c1ccnc(NC(=O)[C@H](NC(=O)c2nocc2CC)C2CCC(F)(F)CC2)c1)N1C[C@@H](C(F)(F)F)NC1=O]

Following the procedure described for Example 33, using 50 mg (S)-2-amino-2-(4,4-difluorocyclohexyl)-N-(4-((R)-1-((S)-2-oxo-4-(trifluoromethyl)imidazolidin-1-yl)butyl)-pyridin-2-yl)acetamide (Intermediate 61), 20 mg of the title compound was obtained. LC/MS: m/z=601.3 [M+H]⁺; rt: 2.31 min (LC/MS-method A). ¹H NMR (400 MHz, DMSO-de) 6 ppm: 10.79 (s, 1H), 8.87 (t, J=0.9 Hz, 1H), 8.73 (d, J=8.2 Hz, 1H), 8.31 (d, J=5.2 Hz, 1H), 8.02 (br s, 1H), 7.54 (d, J=2.4 Hz, 1H), 7.03 (dd, J=5.2, 1.6 Hz, 1H), 4.81 (dd, J=10.0, 5.8 Hz, 1H), 4.71-4.65 (m, 1H), 4.47-4.36 (m, 1H), 3.70-3.63 (m, 1H), 3.05 (dd, J=10.0, 4.6 Hz, 1H), 2.60-2.51 (m, 2H, partially overlapping solvent), 2.11-1.96 (m, 3H), 1.94-1.63 (m, 6H), 1.54-1.18 (m, 4H), 1.12 (t, J=7.5 Hz, 3H), 0.94 (t, J=7.3 Hz, 3H).

### Example 159: N—((S)-1-(4,4-difluorocyclohexyl)-2-((4-((R)-2-methyl-1-((S)-2-oxo-4-(trifluoromethyl)imidazolidin-1-yl)propyl)pyridin-2-yl)amino)-2-oxoethyl)-4-ethylisoxazole-3-carboxamide

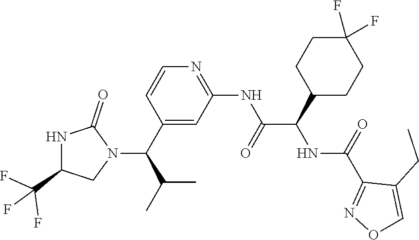 [SMILES: CCc1conc1C(=O)N[C@@H](C(=O)Nc1cc([C@@H](C(C)C)N2C[C@@H](C(F)(F)F)NC2=O)ccn1)C1CCC(F)(F)CC1]

The title compound was prepared, following the procedure described for Example 158 using isopropylmagnesiumchloride in the first step, to afford 20 mg as a colorless solid. LC/MS: m/z=601.3 [M+H]⁺; rt: 2.28 min (LC/MS-method A). ¹H NMR (400 MHz, DMSO-de) 6 ppm: 10.80 (s, 1H), 8.87 (t, J=1.0 Hz, 1H), 8.75 (d, J=8.3 Hz, 1H), 8.33 (d, J=5.2 Hz, 1H), 8.00 (br s, 1H), 7.46 (d, J=2.4 Hz, 1H), 7.13 (dd, J=5.2, 1.5 Hz, 1H), 4.71-4.64 (m, 1H), 4.46-4.36 (m, 1H), 4.31 (d, J=11.3 Hz, 1H), 3.76-3.68 (m, 1H, partially overlapping water), 3.11 (dd, J=9.9, 5.0 Hz, 1H), 2.58-2.51 (m, 2H, partially overlapping solvent), 2.47-2.36 (m, 1H), 2.11-1.96 (m, 3H), 1.91-1.63 (m, 4H), 1.53-1.26 (m, 2H), 1.12 (t, J=7.5 Hz, 3H), 0.95 (d, J=6.5 Hz, 3H), 0.73 (d, J=6.5 Hz, 3H).

### Example 160: N-((1S)-2-((4-(cyclopropyl(3,3,3-trifluoropropanamido)methyl)pyridin-2-yl)amino)-1-(4,4-difluorocyclohexyl)-2-oxoethyl)-1-isopropyl-1H-pyrazole-5-carboxamide

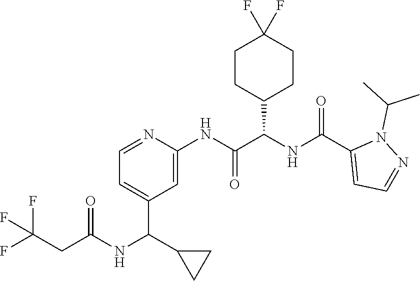 [SMILES: CC(C)n1nccc1C(=O)N[C@H](C(=O)Nc1cc(C(NC(=O)CC(F)(F)F)C2CC2)ccn1)C1CCC(F)(F)CC1]

Following the procedure described for Example 33, using 30 mg N-((2-((S)-2-amino-2-(4,4-difluorocyclohexyl)acetamido)pyridin-4-yl)(cyclopropyl)methyl)-3,3,3-trifluoropropanamide (Intermediate 63), 16 mg of the title compound was obtained. LC/MS: m/z=585.3 [M+H]⁺; rt: 2.14 min (LC/MS-method A). ¹H NMR (400 MHz, DMSO-d₆) δ ppm: 10.75 (br s, 1H), 8.97 (d, J=7.6 Hz, 1H), 8.57 (d, J=8.0 Hz, 1H), 8.28 (d, J=5.2 Hz, 1H), 8.09 (br s, 1H), 7.51 (d, J=2.0 Hz, 1H), 7.12 (dd, J=5.2, 1.6 Hz, 1H), 6.94 (d, J=2.0 Hz, 1H), 5.36 (hept, J=6.6 Hz, 1H), 4.65-4.58 (m, 1H), 4.22-4.15 (m, 1H), 3.38-3.25 (m, 2H), 2.13-1.95 (m, 3H), 1.95-1.63 (m, 4H), 1.55-1.29 (m, 8H), 1.15-1.05 (m, 1H), 0.58-0.48 (m, 2H), 0.46-0.30 (m, 2H).

### Example 161: N-((1S)-2-((4-(cyclopropyl(4,4,4-trifluorobutanamido)methyl)pyridin-2-yl)amino)-1-(4,4-difluorocyclohexyl)-2-oxoethyl)-3-ethylisoxazole-4-carboxamide

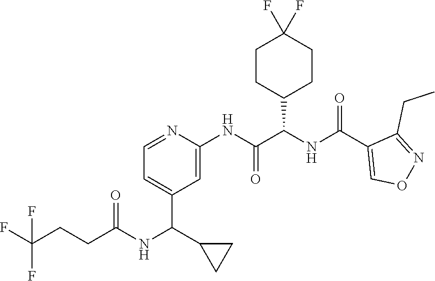 [SMILES: CCc1nocc1C(=O)N[C@H](C(=O)Nc1cc(C(NC(=O)CCC(F)(F)F)C2CC2)ccn1)C1CCC(F)(F)CC1]

Following the procedure described for Example 33, using 30 mg N-((2-((S)-2-amino-2-(4,4-difluorocyclohexyl)acetamido)pyridin-4-yl)(cyclopropyl)methyl)-4,4,4-trifluorobutanamide (Intermediate 41), 27 mg of the title compound was obtained. LC/MS: m/z=586.3 [M+H]⁺; rt: 2.16 min (LC/MS-method A). ¹H NMR (400 MHz, DMSO-de) 5 ppm: 1H NMR (400 Mhz, DMSO) δ ppm 10.81 (br s, 1H), 9.42 (s, 1H), 8.74 (d, J=7.7 Hz, 1H), 8.56 (d, J=8.1 Hz, 1H), 8.27 (d, J=5.3 Hz, 1H), 8.08 (br s, 1H), 7.14 (dd, J=5.3, 1.6 Hz, 1H), 4.69-4.62 (m, 1H), 4.20-4.13 (m, 1H), 2.90-2.77 (m, 2H), 2.55-2.36 (m, 4H, partially overlapping solvent), 2.13-1.63 (m, 7H), 1.55-1.30 (m, 2H), 1.16 (t, J=7.5 Hz, 3H), 1.13-1.04 (m, 1H), 0.57-0.47 (m, 2H), 0.44-0.30 (m, 2H).

### Example 162: 4-Cyclopropyl-N—((S)-2-((4-((R or S)-cyclopropyl((S)-2-oxo-4-(trifluoromethyl)imidazolidin-1-yl)methyl)pyridin-2-yl)amino)-1-(4,4-difluorocyclohexyl)-2-oxoethyl)-1,2,5-oxadiazole-3-carboxamide-DS1

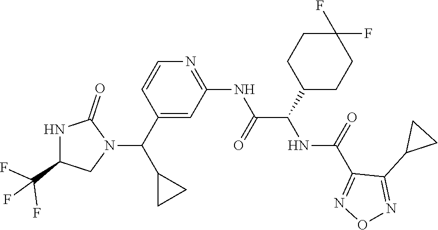 [SMILES: O=C(N[C@H](C(=O)Nc1cc(C(C2CC2)N2C[C@@H](C(F)(F)F)NC2=O)ccn1)C1CCC(F)(F)CC1)c1nonc1C1CC1]

To a mixture of (2S)-2-amino-N-(4-((R or S)-cyclopropyl((S)-2-oxo-4-(trifluoromethyl)imidazolidin-1-yl)methyl)pyridin-2-yl)-2-(4,4-difluorocyclohexyl)acetamide hydrochloride (Int. 48-dS1, 20 mg), and 4-cyclopropyl-1,2,5-oxadiazole-3-carboxylic acid (7 mg) in DCM (1 ml), DIPEA (34 μl) and T3P (70 μl) were added. After stirring for 1 h the mixture was concentrated in vacuo and the residue was purified by preparative RP HPLC (Agilent Prep 018, 21.2 mm×250 mm, 10 μm; 50 ml/min, from 90% H₂O/10% ACN to 10% H₂O/90% ACN in 12.5 min). The pure product containing fractions were combined, the ACN was partly removed and the residue freeze dried to yield 15 mg of the title compound. LC/MS: m/z=612.2 [M+H]; rt: 2.38 min (LC/MS-method A). ¹H NMR (400 MHz, DMSO-d)) δ ppm: 10.83 (s, 1H), 9.30 (d, J=8.0 Hz, 1H), 8.32 (d, J=5.2 Hz, 1H), 8.19 (br s, 1H), 7.56 (d, J=2.2 Hz, 1H), 7.15 (br d, J=5.2 Hz, 1H), 4.80-4.73 (m, 1H), 4.47-4.36 (m, 1H), 4.02 (d, J=10.2 Hz, 1H), 3.58 (dd, J=10.0, 4.7 Hz, 1H), 3.54-3.47 (m, 1H), 2.28 (tt, J=8.4, 5.0 Hz, 1H), 2.12-1.97 (m, 3H), 1.92-1.65 (m, 4H), 1.56-1.30 (m, 3H), 1.16-1.10 (m, 2H), 1.01-0.95 (m, 2H), 0.77-0.68 (m, 1H), 0.60-0.45 (m, 2H), 0.30-0.22 (m, 1H).

Using (2S)-2-amino-N-(4-((R or S)-cyclopropyl((S)-2-oxo-4-(trifluoromethyl)-imidazolidin-1-yl)methyl)pyridin-2-yl)-2-(4,4-difluorocyclohexyl)acetamide hydrochloride (Intermediate 48-DS2, 20 mg) as starting material, analogously to example 162 were prepared:

| Ex. | Acid | Structure | Name | Yield |
|---|---|---|---|---|
| 163 | | | 4-Cyclopropyl-N-((S)-2-((4-((R | 16 mg, |
| | | | or S)-cyclopropyl((S)-2-oxo-4- | 67% |
| | | | (trifluoromethyl)imidazolidin-1- | |
| | | | yl)methyl)pyridin-2-yl) amino)-1- | |
| | | | (4,4-difluorocyclo hexyl)-2- | |
| | | | oxoethyl)-1,2,5-oxadiazole-3- | |
| | | | carboxamide-DS2 | |
| 164 | | | N-((1S)-2-((4-((R or S)- | 12 mg, |
| | | | cyclopropyl((S)-2-oxo-4- | 51% |
| | | | (trifluoromethyl)imidazolidin-1- | |
| | | | yl)methyl)pyridin-2-yl)amino)-1- | |
| | | | (4,4-difluoro cyclohexyl)-2- | |
| | | | oxoethyl)-4-methyl-1,2,5- | |
| | | | oxadiazole-3-carboxamide-DS2 | |
| 165 | | | N-((1S)-2-((4-((R or S)- | 6 mg, |
| | | | cyclopropyl((S)-2-oxo-4- | 27% |
| | | | (trifluoromethyl)imidazolidin-1- | |
| | | | yl)methyl)pyridin-2-yl)amino)-1- | |
| | | | (4,4-difluoro cyclohexyl)-2- | |
| | | | oxoethyl)-1-ethyl-1H-pyrazole- | |
| | | | 5-carboxamide-DS2 | |
| 166 | | | N-((1S)-2-((4-((R or S)- | 9 mg, |
| | | | cyclopropyl((S)-2-oxo-4- | 41% |
| | | | (trifluoromethyl) imidazolidin-1- | |
| | | | yl)methyl) pyridin-2-yl)amino)-1- | |
| | | | (4,4-difluorocyclohexyl)-2- | |
| | | | oxoethyl)-1-methyl-1H-pyrazole- | |
| | | | 5-carboxamide-DS2 | |

### Example 163

LC/MS: m/z=612.2 [M+H]⁺; rt: 2.29 min (LC/MS-method A). ¹H NMR (400 MHz, DMSO-d₆) δ ppm: 10.80 (s, 1H), 9.30 (d, J=8.1 Hz, 1H), 8.32 (d, J=5.2 Hz, 1H), 8.17 (br s, 1H), 7.63 (d, J=2.3 Hz, 1H), 7.09 (br d, J=5.2 Hz, 1H), 4.79-4.72 (m, 1H), 4.52-4.41 (m, 1H), 4.04 (d, J=10.3 Hz, 1H), 3.90-3.83 (m, 1H), 3.23 (dd, J=10.1, 3.6 Hz, 1H), 2.27 (tt, J=8.4, 5.0 Hz, 1H), 2.12-1.98 (m, 3H), 1.92-1.65 (m, 4H), 1.56-1.30 (m, 3H), 1.16-1.09 (m, 2H), 1.00-0.95 (m, 2H), 0.80-0.71 (m, 1H), 0.63-0.48 (m, 2H), 0.37-0.29 (m, 1H).

### Example 164

LC/MS: m/z=586.2 [M+H]⁺; rt: 2.11 min (LC/MS-method A). ¹H NMR (400 MHz, DMSO-d₆) δ ppm: 10.78 (s, 1H), 9.21 (d, J=8.1 Hz, 1H), 8.32 (d, J=5.2 Hz, 1H), 8.16 (br s, 1H), 7.63 (d, J=2.3 Hz, 1H), 7.09 (br d, J=5.2 Hz, 1H), 4.76-4.69 (m, 1H), 4.52-4.41 (m, 1H), 4.04 (d, J=10.2 Hz, 1H), 3.90-3.82 (m, 1H), 3.23 (dd, J=10.1, 3.6 Hz, 1H), 2.47 (s, 3H), 2.12-1.97 (m, 3H), 1.92-1.64 (m, 4H), 1.55-1.29 (m, 3H), 0.80-0.71 (m, 1H), 0.63-0.48 (m, 2H), 0.37-0.29 (m, 1H).

### Example 165

LC/MS: m/z=598.2 [M+H]⁺; rt: 2.01 min (LC/MS-method A). ¹H NMR (400 MHz, DMSO-de) δ ppm: 10.69 (s, 1H), 8.55 (d, J=8.1 Hz, 1H), 8.31 (d, J=5.2 Hz, 1H), 8.17 (br s, 1H), 7.62 (d, J=2.3 Hz, 1H), 7.48 (d, J=2.1 Hz, 1H), 7.08 (br d, J=5.2 Hz, 1H), 7.00 (d, J=2.1 Hz, 1H), 4.69-4.62 (m, 1H), 4.51-4.40 (m, 3H), 4.03 (d, J=10.3 Hz, 1H), 3.89-3.82 (m, 1H), 3.22 (dd, J=10.1, 3.6 Hz, 1H), 2.12-1.95 (m, 3H), 1.94-1.63 (m, 4H), 1.55-1.23 (m, 6H), 0.80-0.71 (m, 1H), 0.62-0.47 (m, 2H), 0.36-0.29 (m, 1H).

### Example 166

LC/MS: m/z=584.2 [M+H]⁺; rt: 1.89 min (LC/MS-method A). ¹H NMR (400 MHz, DMSO-de) δ ppm: 10.70 (s, 1H), 8.55 (d, J=8.1 Hz, 1H), 8.31 (d, J=5.2 Hz, 1H), 8.17 (br s, 1H), 7.62 (d, J=2.3 Hz, 1H), 7.47 (d, J=2.1 Hz, 1H), 7.08 (br d, J=5.2 Hz, 1H), 7.04 (d, J=2.1 Hz, 1H), 4.69-4.63 (m, 1H), 4.52-4.41 (m, 1H), 4.06-4.00 (m, 4H), 3.89-3.82 (m, 1H), 3.22 (dd, J=10.0, 3.7 Hz, 1H), 2.13-1.95 (m, 3H), 1.94-1.62 (m, 4H), 1.55-1.23 (m, 3H), 0.80-0.71 (m, 1H), 0.62-0.47 (m, 2H), 0.37-0.28 (m, 1H).

### Example 167: N—((S)-1-(4,4-difluorocyclohexyl)-2-oxo-2-((4-((R)-1-(4,4,4-trifluoro-butanamido)ethyl)pyridin-2-yl)amino)ethyl)-1-isopropyl-1H-pyrazole-5-carboxamide

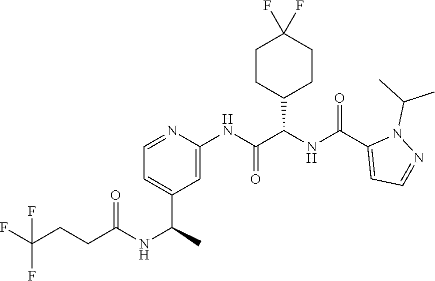 [SMILES: CC(C)n1nccc1C(=O)N[C@H](C(=O)Nc1cc([C@@H](C)NC(=O)CCC(F)(F)F)ccn1)C1CCC(F)(F)CC1]

Following the procedure described for Example 33, using 35 mg N-(1-(2-((S)-2-amino-2-(4,4-difluorocyclohexyl)acetamido)pyridin-4-yl)ethyl)-4,4,4-trifluorobutanamide ((Intermediate 64), 14 mg of the title compound was obtained. LC/MS: m/z=573.3 [M+H]⁺; rt: 2.10 min (LC/MS-method A). ¹H NMR (400 MHz, DMSO-d₆) δ ppm: 10.78 (br s, 1H), 8.62-8.56 (m, 2H), 8.26 (d, J=5.3 Hz, 1H), 8.01 (br s, 1H), 7.51 (d, J=2.0 Hz, 1H), 7.09 (dd, J=5.3, 1.6 Hz, 1H), 6.93 (d, J=2.0 Hz, 1H), 5.36 (hept, J=6.6 Hz, 1H), 4.90-4.81 (m, 1H), 4.63-4.56 (m, 1H), 2.55-2.36 (m, 4H, overlapping solvent), 2.13-1.62 (m, 7H), 1.55-1.42 (m, 1H), 1.42-1.29 (m, 10H).

### Example 168: 4-Cyclopropyl-N—((S)-1-(4,4-difluorocyclohexyl)-2-((4-((R)-1-(4-methyl-2-oxo-2,3-dihydro-1H-imidazol-1-yl)ethyl)pyridin-2-yl)amino)-2-oxoethyl)-1,2,5-oxadiazole-3-carboxamide

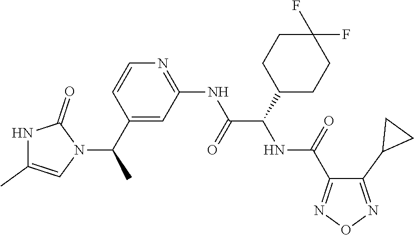 [SMILES: Cc1cn([C@H](C)c2ccnc(NC(=O)[C@@H](NC(=O)c3nonc3C3CC3)C3CCC(F)(F)CC3)c2)c(=O)[nH]1]

Following Example 162, using (S)-2-amino-2-(4,4-difluorocyclohexyl)-N-(4-((R)-1-(4-methyl-2-oxo-2,3-dihydro-1H-imidazol-1-yl)ethyl)pyridin-2-yl)acetamide (Intermediate 49, 18 mg) and stirring for 20 min, yielded 13 mg (53%) of the title compound. LC/MS: m/z=530.2 [M+H]⁺; rt: 2.05 min (LC/MS-method A). ¹H NMR (400 MHz, DMSO-d₆) δ ppm: 10.79 (s, 1H), 9.96 (d, J=2.1 Hz, 1H), 9.29 (d, J=8.0 Hz, 1H), 8.28 (d, J=5.2 Hz, 1H), 7.99 (br s, 1H), 6.99 (dd, J=5.2, 1.6 Hz, 1H), 6.25-6.22 (m, 1H), 5.15 (q, J=7.2 Hz, 1H), 4.77-4.70 (m, 1H), 2.27 (tt, J=8.4, 5.0 Hz, 1H), 2.11-1.97 (m, 3H), 1.94-1.63 (m, 7H), 1.58-1.29 (m, 5H), 1.16-1.10 (m, 2H), 1.00-0.94 (m, 2H).

### Example 169: N-((1S)-1-(4,4-Difluorocyclohexyl)-2-oxo-2-((4-(R or S)-(1-((S)-2-oxo-4-(trifluoromethyl)imidazolidin-1-yl)ethyl)pyridin-2-yl)amino)ethyl)-4-methyl-1,2,5-oxadiazole-3-carboxamide—DS1

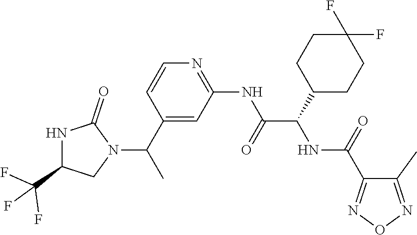 [SMILES: Cc1nonc1C(=O)N[C@H](C(=O)Nc1cc(C(C)N2C[C@@H](C(F)(F)F)NC2=O)ccn1)C1CCC(F)(F)CC1]

Following Example 162, using (S)-2-amino-2-(4,4-difluorocyclohexyl)-N-(4-((R or S)-1-((S)-2-oxo-4-(trifluoromethyl)imidazolidin-1-yl)ethyl)pyridin-2-yl)acetamide hydrochloride (Intermediate 50-DS1, 14 mg) and 4-methyl-1,2,5-oxadiazole-3-carboxylic acid (4 mg), yielded 9 mg (58%) of the title compound. LC/MS: m/z=560.1 [M+H]⁺; rt: 1.99 min (LC/MS-method A). ¹H NMR (400 MHz, DMSO-d₆) δ ppm: 10.79 (s, 1H), 9.21 (d, J=8.0 Hz, 1H), 8.31 (d, J=5.2 Hz, 1H), 8.02 (br s, 1H), 7.59 (d, J=2.4 Hz, 1H), 7.01 (dd, J=5.2, 1.6 Hz, 1H), 4.97 (q, J=7.1 Hz, 1H), 4.75-4.68 (m, 1H), 4.45-4.34 (m, 1H), 3.75-3.67 (m, 1H), 3.08 (dd, J=10.0, 4.2 Hz, 1H), 2.47 (s, 3H), 2.12-1.97 (m, 3H), 1.92-1.64 (m, 4H), 1.54-1.29 (m, 5H).

Using (S)-2-amino-2-(4,4-difluorocyclohexyl)-N-(4-((R or S)-1-((S)-2-oxo-4-(trifluoromethyl)imidazolidin-1-yl)ethyl)pyridin-2-yl)acetamide hydrochloride (Intermediate 50-DS1) as starting material, analogously to example 169 were prepared:

| Ex. | Acid | Structure | Name | Yield |
|---|---|---|---|---|
| 170 | | | 4-cyclopropyl-N-((S)-1-(4,4- | 10 mg, |
| | | | difluorocyclohexyl)-2-oxo-2- | 58%) |
| | | | ((4-((R or S)-1-((S)-2-oxo-4- | |
| | | | (trifluoromethyl)imidazolidin- | |
| | | | 1-yl)ethyl)pyridin-2-yl)amino) | |
| | | | ethyl)-1,2,5-oxadiazole-3- | |
| | | | carboxamide-DS1 | |
| 171 | | | N-((1S)-1-(4,4-difluorocyclo | 9 mg, |
| | | | hexyl)-2-oxo-2-((4-((R or S)- | 58% |
| | | | 1-((S)-2-oxo-4- | |
| | | | (trifluoromethyl) imidazolidin- | |
| | | | 1-yl)ethyl) pyridin-2- | |
| | | | yl)amino) ethyl)-1-methyl- | |
| | | | 1H-pyrazole-5-carboxamide- | |
| | | | DS1 | |

### Example 170

LC/MS: m/z=586.1 [M+H]⁺; rt: 2.17 min (LC/MS-method A). ¹H NMR (400 MHz, DMSO-de) δ ppm: 10.80 (s, 1H), 9.29 (d, J=8.0 Hz, 1H), 8.31 (d, J=5.2 Hz, 1H), 8.02 (br s, 1H), 7.59 (d, J=2.4 Hz, 1H), 7.01 (dd, J=5.2, 1.6 Hz, 1H), 4.97 (q, J=7.1 Hz, 1H), 4.78-4.70 (m, 1H), 4.46-4.34 (m, 1H), 3.76-3.67 (m, 1H), 3.08 (dd, J=10.0, 4.2 Hz, 1H), 2.27 (tt, J=8.4, 5.0 Hz, 1H), 2.12-1.97 (m, 3H), 1.92-1.64 (m, 4H), 1.56-1.30 (m, 5H), 1.17-1.08 (m, 2H), 1.01-0.93 (m, 2H).

### Example 171

LC/MS: m/z=558.1 [M+H]⁺; rt: 1.75 min (LC/MS-method A). ¹H NMR (400 MHz, DMSO-de) δ ppm: 10.70 (s, 1H), 8.55 (d, J=8.0 Hz, 1H), 8.30 (d, J=5.2 Hz, 1H), 8.03 (br s, 1H), 7.58 (d, J=2.4 Hz, 1H), 7.47 (d, J=2.1 Hz, 1H), 7.04 (d, J=2.1 Hz, 1H), 6.99 (dd, J=5.2, 1.6 Hz, 1H), 4.96 (q, J=7.1 Hz, 1H), 4.68-4.61 (m, 1H), 4.45-4.34 (m, 1H), 4.02 (s, 3H), 3.75-3.66 (m, 1H), 3.07 (dd, J=10.0, 4.2 Hz, 1H), 2.12-1.94 (m, 3H), 1.94-1.61 (m, 4H), 1.55-1.28 (m, 5H).

### Example 172: N—((S)-1-(4,4-difluorocyclohexyl)-2-oxo-2-((4-(2-((S)-2-oxo-4-(trifluoro-methyl)imidazolidin-1-yl)propan-2-yl)pyridin-2-yl)amino)ethyl)-1-methyl-1H-pyrazole-5-carboxamide

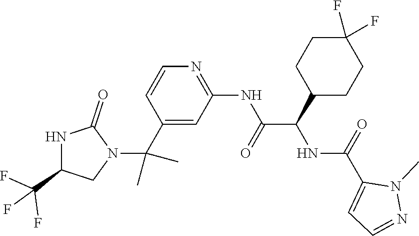 [SMILES: Cn1nccc1C(=O)N[C@@H](C(=O)Nc1cc(C(C)(C)N2C[C@@H](C(F)(F)F)NC2=O)ccn1)C1CCC(F)(F)CC1]

Following the procedure described for Example 33, using 28 mg N-(1-(2-((S)-2-amino-2-(4,4-difluorocyclohexyl)acetamido)pyridin-4-yl)ethyl)-4,4,4-trifluorobutanamide (Intermediate 65), 34 mg of the title compound was obtained. LC/MS: m/z=570.3 [M+H]⁺; rt: 1.85 min (LC/MS-method A). ¹H NMR (400 MHz, DMSO-d₆) δ ppm: 11.03 (br s, 1H), 8.63 (d, J=7.9 Hz, 1H), 8.26 (d, J=5.6 Hz, 1H), 8.03 (d, J=1.7 Hz, 1H), 7.50-7.46 (m, 2H), 7.15 (dd, J=5.6, 1.7 Hz, 1H), 7.06 (d, J=2.1 Hz, 1H), 4.66-4.59 (m, 1H), 4.43-4.33 (m, 1H), 4.02 (s, 3H), 3.91-3.84 (m, 1H), 3.62-3.52 (m, 1H, overlapping water), 2.13-1.98 (m, 3H), 1.95-1.65 (m, 4H), 1.59-1.29 (m, 8H).

Examples 173-175 were prepared analogously to Example 172.

| Ex. | Acid | Structure | Name | Yield |
|---|---|---|---|---|
| 173 | | | N-((S)-1-(4,4- | 13 mg, |
| | | | difluorocyclohexyl)-2-oxo-2- | 36% |
| | | | ((4-(2-((S)-2-oxo-4- | |
| | | | (trifluoromethyl)imidazolidin-1- | |
| | | | yl)propan-2-yl)pyridin-2- | |
| | | | yl)amino)ethyl)-3- | |
| | | | ethylisoxazole-4-carboxamide | |
| 174 | | | N-((S)-1-(4,4- | 34 mg, |
| | | | difluorocyclohexyl)-2-oxo-2- | 96% |
| | | | ((4-(2-((S)-2-oxo-4- | |
| | | | (trifluoromethyl)imidazolidin-1- | |
| | | | yl)propan-2-yl)pyridin-2- | |
| | | | yl)amino)ethyl)-1-ethyl-1H- | |
| | | | pyrazole-5-carboxamide | |
| 175 | | | N-((S)-1-(4,4- | 35 mg, |
| | | | difluorocyclohexyl)-2-oxo-2- | 96% |
| | | | ((4-(2-((S)-2-oxo-4- | |
| | | | (trifluoromethyl)imidazolidin-1- | |
| | | | yl)propan-2-yl)pyridin-2- | |
| | | | yl)amino)ethyl)-1-isopropyl- | |
| | | | 1H-pyrazole-5-carboxamide | |

### Example 173

LC/MS: m/z=587.3 [M+H]⁺; rt: 2.02 min (LC/MS-method A). ¹H NMR (400 MHz, DMSO-d6) δ ppm: 10.93 (br s, 1H), 9.43 (s, 1H), 8.58 (d, J=8.0 Hz, 1H), 8.25 (d, J=5.5 Hz, 1H), 8.05 (d, J=1.7 Hz, 1H), 7.47 (d, J=2.3 Hz, 1H), 7.12 (dd, J=5.5, 1.7 Hz, 1H), 4.70-4.63 (m, 1H), 4.42-4.32 (m, 1H), 3.91-3.83 (m, 1H), 3.60-3.54 (m, 1H, overlapping water), 2.87-2.79 (m, 2H), 2.13-1.65 (m, 7H), 1.60-1.30 (m, 8H), 1.17 (t, J=7.5 Hz, 3H).

### Example 174

LC/MS: m/z=586.3 [M+H]⁺; rt: 1.95 min (LC/MS-method A). ¹H NMR (400 MHz, DMSO-d6) δ ppm: 11.12 (br s, 1H), 8.65 (d, J=7.9 Hz, 1H), 8.27 (d, J=5.7 Hz, 1H), 8.02 (d, J=1.7 Hz, 1H), 7.51-7.47 (m, 2H), 7.17 (dd, J=5.7, 1.7 Hz, 1H), 7.03 (d, J=2.1 Hz, 1H), 4.65-4.59 (m, 1H), 4.52-4.33 (m, 3H), 3.92-3.84 (m, 1H), 3.66-3.56 (m, 1H, overlapping water), 2.13-1.99 (m, 3H), 1.96-1.67 (m, 4H), 1.60-1.23 (m, 11H).

### Example 175

LC/MS: m/z=604.4 [M+H]⁺; rt: 2.07 min (LC/MS-method A). ¹H NMR (400 MHz, DMSO-d6) δ ppm: 11.04 (br s, 1H), 8.62 (d, J=7.9 Hz, 1H), 8.26 (d, J=5.6 Hz, 1H), 8.02 (d, J=1.7 Hz, 1H), 7.52-7.46 (m, 2H), 7.15 (dd, J=5.6, 1.7 Hz, 1H), 6.96 (d, J=2.0 Hz, 1H), 5.37 (hept, J=6.6 Hz, 1H), 4.65-4.57 (m, 1H), 4.43-4.33 (m, 1H), 3.92-3.84 (m, 1H), 3.64-3.53 (m, 1H, overlapping water), 2.13-1.98 (m, 3H), 1.96-1.66 (m, 4H), 1.59-1.31 (m, 14H).

### Example 176: N—((S)-2-((4-((S)-1-(5,5-difluoro-2-oxotetrahydropyrimidin-1(2H)-yl)-2-methoxyethyl)pyridin-2-yl)amino)-1-(4,4-difluorocyclohexyl)-2-oxoethyl)-5-ethylisoxazole-4-carboxamide

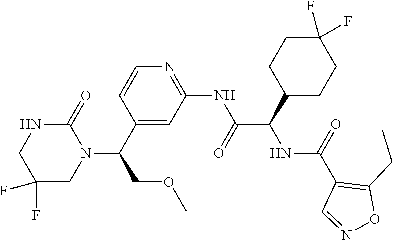 [SMILES: CCc1oncc1C(=O)N[C@@H](C(=O)Nc1cc([C@@H](COC)N2CC(F)(F)CNC2=O)ccn1)C1CCC(F)(F)CC1]

Following the procedure described for Example 33, using 32 mg (S)-2-amino-N-(4-((S)-1-(5,5-difluoro-2-oxotetrahydropyrimidin-1(2H)-yl)-2-methoxyethyl)pyridin-2-yl)-2-(4,4-difluorocyclohexyl)acetamide (Intermediate 67), 34 mg of the title compound was obtained. LC/MS: m/z=585.2 [M+H]⁺; rt: 1.93 min (LC/MS-method A). ¹H NMR (400 MHz, DMSO-de) δ ppm: 10.74 (s, 1H), 9.08 (s, 1H), 8.45 (d, J=8.1 Hz, 1H), 8.30 (d, J=5.3 Hz, 1H), 8.06 (br s, 1H), 7.06 (dd, J=5.3, 1.6 Hz, 1H), 6.90 (br s, 1H), 5.52-5.47 (m, 1H), 4.71-4.65 (m, 1H), 3.85-3.77 (m, 2H), 3.72-3.33 (m, 4H, partially overlapping water), 3.31 (s, 3H), 3.07 (q, J=7.6 Hz, 2H), 2.13-1.63 (m, 7H), 1.55-1.29 (m, 2H), 1.19 (t, J=7.6 Hz, 3H).

Examples 177-181 were prepared analogously to Example 176. Examples 182-183 were prepared analogously to Example 176 using the procedure described for Example 39.

| Ex. | Acid | Structure | Name | Yield |
|---|---|---|---|---|
| 177 | 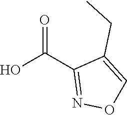 | 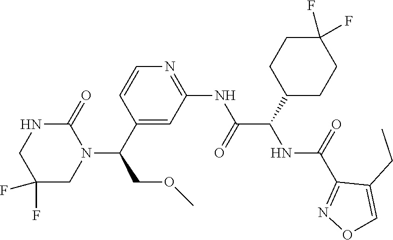 [SMILES: CCc1conc1C(=O)N[C@H](C(=O)Nc1cc([C@@H](COC)N2CC(F)(F)CNC2=O)ccn1)C1CCC(F)(F)CC1] | N-((S)-2-((4-((S)-1-(5,5-difluoro- 2-oxotetrahydropyrimidin-1(2H)- yl)-2-methoxyethyl)pyridin-2- yl)amino)-1-(4,4- difluorocyclohexyl)-2-oxoethyl)- 4-ethylisoxazole-3-carboxamide | 25 mg, 62% |
| 178 | 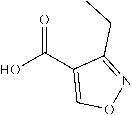 | 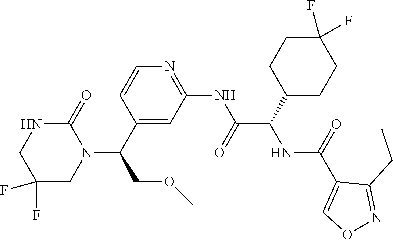 [SMILES: CCc1nocc1C(=O)N[C@H](C(=O)Nc1cc([C@@H](COC)N2CC(F)(F)CNC2=O)ccn1)C1CCC(F)(F)CC1] | N-((S)-2-((4-((S)-1-(5,5-difluoro- 2-oxotetrahydropyrimidin-1(2H)- yl)-2-methoxyethyl)pyridin-2- yl)amino)-1-(4,4- difluorocyclohexyl)-2-oxoethyl)- 3-ethylisoxazole-4-carboxamide | 20 mg, 49% |
| 179 | 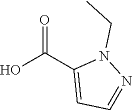 | 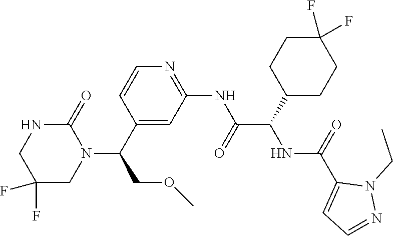 [SMILES: CCn1nccc1C(=O)N[C@H](C(=O)Nc1cc([C@@H](COC)N2CC(F)(F)CNC2=O)ccn1)C1CCC(F)(F)CC1] | N-((S)-2-((4-((S)-1-(5,5-difluoro- 2-oxotetrahydropyrimidin-1(2H)- yl)-2-methoxyethyl)pyridin-2- yl)amino)-1-(4,4- difluorocyclohexyl)-2-oxoethyl)- 1-ethyl-1H-pyrazole-5- carboxamide | 35 mg, 86% |
| 180 | 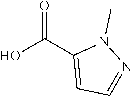 | 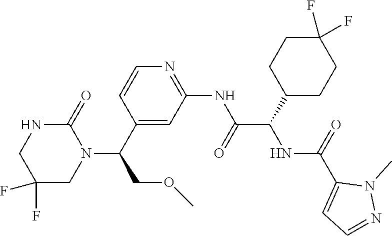 [SMILES: COC[C@H](c1ccnc(NC(=O)[C@@H](NC(=O)c2ccnn2C)C2CCC(F)(F)CC2)c1)N1CC(F)(F)CNC1=O] | N-((S)-2-((4-((S)-1-(5,5-difluoro- 2-oxotetrahydropyrimidin-1(2H)- yl)-2-methoxyethyl)pyridin-2- yl)amino)-1-(4,4- difluorocyclohexyl)-2-oxoethyl)- 1-methyl-1H-pyrazole-5- carboxamide | 36 mg, 91% |
| 181 | 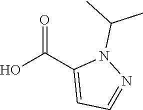 | 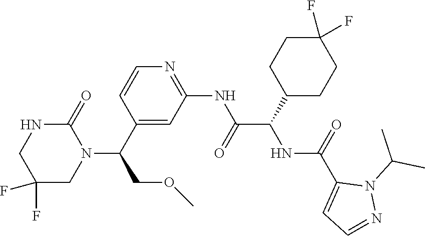 [SMILES: COC[C@H](c1ccnc(NC(=O)[C@@H](NC(=O)c2ccnn2C(C)C)C2CCC(F)(F)CC2)c1)N1CC(F)(F)CNC1=O] | N-((S)-2-((4-((S)-1-(5,5-difluoro- 2-oxotetrahydropyrimidin-1(2H)- yl)-2-methoxyethyl)pyridin-2- yl)amino)-1-(4,4- difluorocyclohexyl)-2-oxoethyl)- 1-isopropyl-1H-pyrazole-5- carboxamide | 37 mg, 89% |
| 182 | 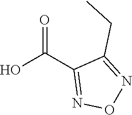 | 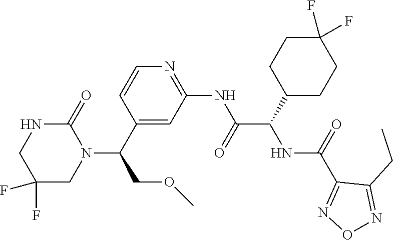 [SMILES: CCc1nonc1C(=O)N[C@H](C(=O)Nc1cc([C@@H](COC)N2CC(F)(F)CNC2=O)ccn1)C1CCC(F)(F)CC1] | N-((S)-2-((4-((S)-1-(5,5-difluoro- 2-oxotetrahydropyrimidin-1(2H)- yl)-2-methoxyethyl)pyridin-2- yl)amino)-1-(4,4- difluorocyclohexyl)-2-oxoethyl)- 4-ethyl-1,2,5-oxadiazole-3- carboxamide | 34 mg, 81% |
| 183 | | | N-((S)-2-((4-((S)-1-(5,5-difluoro- 2-oxotetrahydropyrimidin-1(2H)- yl)-2-methoxyethyl)pyridin-2- yl)amino)-1-(4,4- difluorocyclohexyl)-2-oxoethyl)- 4-ethyl-1,2,5-oxadiazole-3- carboxamide | 32 mg, 78% |

### Example 177

LC/MS: m/z=585.2 [M+H]⁺; rt: 2.04 min (LC/MS-method A). ¹H NMR (400 MHz, DMSO-d6) δ ppm: 10.76 (s, 1H), 8.87 (t, J=1.0 Hz, 1H), 8.74 (d, J=8.3 Hz, 1H), 8.30 (d, J=5.3 Hz, 1H), 8.05 (br s, 1H), 7.06 (dd, J=5.3, 1.6 Hz, 1H), 6.90 (br s, 1H), 5.53-5.47 (m, 1H), 4.72-4.66 (m, 1H), 3.86-3.77 (m, 2H), 3.73-3.34 (m, 4H, partially overlapping water), 3.32 (s, 3H), 2.59-2.51 (m, 2H), 2.11-1.97 (m, 3H), 1.90-1.65 (m, 4H), 1.52-1.27 (m, 2H), 1.13 (t, J=7.5 Hz, 3H).

### Example 178

LC/MS: m/z=583.2 [M+H]⁺; rt: 1.90 min (LC/MS-method A). ¹H NMR (400 MHz, DMSO-d6) δ ppm: 10.74 (s, 1H), 9.41 (s, 1H), 8.53 (d, J=8.1 Hz, 1H), 8.30 (d, J=5.3 Hz, 1H), 8.06 (br s, 1H), 7.06 (dd, J=5.3, 1.6 Hz, 1H), 6.90 (br s, 1H), 5.53-5.46 (m, 1H), 4.73-4.66 (m, 1H), 3.85-3.77 (m, 2H), 3.72-3.33 (m, 4H, partially overlapping water), 3.31 (s, 3H), 2.83 (q, J=7.5 Hz, 2H), 2.13-1.63 (m, 7H), 1.54-1.30 (m, 2H), 1.16 (t, J=7.5 Hz, 3H).

### Example 179

LC/MS: m/z=582.2 [M+H]⁺; rt: 1.83 min (LC/MS-method A). ¹H NMR (400 MHz, DMSO-d6) δ ppm: 10.88 (br s, 1H), 8.61 (d, J=8.0 Hz, 1H), 8.31 (d, J=5.3 Hz, 1H), 8.03 (br s, 1H), 7.49 (d, J=2.0 Hz, 1H), 7.09 (dd, J=5.3, 1.6 Hz, 1H), 7.02 (d, J=2.0 Hz, 1H), 6.91 (br s, 1H), 5.53-5.47 (m, 1H), 4.67-4.61 (m, 1H), 4.45 (q, J=7.1 Hz, 2H), 3.86-3.78 (m, 2H), 3.74-3.35 (m, 4H, partially overlapping water), 3.31 (s, 3H), 2.13-1.97 (m, 3H), 1.94-1.64 (m, 4H), 1.55-1.30 (m, 2H), 1.27 (t, J=7.1 Hz, 3H).

### Example 180

LC/MS: m/z=568.1 [M+H]⁺; rt: 1.73 min (LC/MS-method A). ¹H NMR (400 MHz, DMSO-d6) δ ppm: 10.95 (br s, 1H), 8.62 (d, J=8.0 Hz, 1H), 8.31 (d, J=5.4 Hz, 1H), 8.03 (br s, 1H), 7.47 (d, J=2.1 Hz, 1H), 7.10 (dd, J=5.4, 1.6 Hz, 1H), 7.06 (d, J=2.1 Hz, 1H), 6.92 (br s, 1H), 5.53-5.47 (m, 1H), 4.67-4.61 (m, 1H), 4.02 (s, 3H), 3.85-3.79 (m, 2H, partially overlapping water), 3.74-3.35 (m, 4H), 3.31 (s, 3H), 2.13-1.97 (m, 3H), 1.95-1.64 (m, 4H), 1.55-1.42 (m, 1H), 1.42-1.29 (m, 1H).

### Example 181

LC/MS: m/z=596.2 [M+H]⁺; rt: 1.95 min (LC/MS-method A). ¹H NMR (400 MHz, DMSO-d6) δ ppm: 10.85 (br s, 1H), 8.59 (d, J=7.9 Hz, 1H), 8.31 (d, J=5.3 Hz, 1H), 8.03 (br s, 1H), 7.50 (d, J=2.0 Hz, 1H), 7.09 (dd, J=5.3, 1.6 Hz, 1H), 6.95 (d, J=2.0 Hz, 1H), 6.91 (br s, 1H), 5.53-5.47 (m, 1H), 5.37 (hept, J=6.5 Hz, 1H), 4.66-4.59 (m, 1H), 3.86-3.78 (m, 2H), 3.73-3.34 (m, 4H, partially overlapping water), 3.31 (s, 3H), 2.12-1.96 (m, 3H), 1.94-1.64 (m, 4H), 1.55-1.29 (m, 8H).

### Example 182

LC/MS: m/z=586.3 [M+H]⁺; rt: 2.12 min (LC/MS-method A). ¹H NMR (400 MHz, DMSO-d6) δ ppm: 10.90 (s, 1H), 9.29 (d, J=8.1 Hz, 1H), 8.31 (d, J=5.3 Hz, 1H), 8.04 (br s, 1H), 7.09 (dd, J=5.3, 1.6 Hz, 1H), 6.91 (br s, 1H), 5.53-5.47 (m, 1H), 4.76-4.70 (m, 1H), 3.87-3.78 (m, 2H), 3.74-3.35 (m, 4H, overlapping water), 3.32 (s, 3H), 2.90 (q, J=7.5 Hz, 2H), 2.12-1.98 (m, 3H), 1.91-1.65 (m, 4H), 1.55-1.30 (m, 2H), 1.24 (t, J=7.5 Hz, 3H).

### Example 183

LC/MS: m/z=572.3 [M+H]⁺; rt: 1.97 min (LC/MS-method A). ¹H NMR (400 MHz, DMSO-d6) δ ppm: 10.92 (s, 1H), 9.26 (d, J=8.0 Hz, 1H), 8.31 (d, J=5.3 Hz, 1H), 8.04 (br s, 1H), 7.09 (dd, J=5.3, 1.6 Hz, 1H), 6.91 (br s, 1H), 5.53-5.47 (m, 1H), 4.76-4.69 (m, 1H), 3.87-3.78 (m, 2H), 3.74-3.35 (m, 4H, overlapping water), 3.32 (s, 3H), 2.47 (s, 3H), 2.13-1.98 (m, 3H), 1.93-1.65 (m, 4H), 1.55-1.29 (m, 2H).

### Example 184: 4-Cyclopropyl-N—((S)-1-(4,4-difluorocyclohexyl)-2-((4-((R or S)-2-methoxy-1-((3S,5S)-2-oxo-5-(trifluoromethyl)pyrrolidin-3-yl)ethyl)pyridin-2-yl)amino)-2-oxoethyl)-1,2,5-oxadiazole-3-carboxamide—DS1

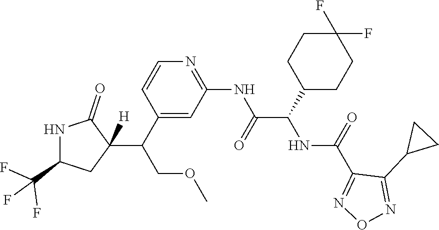 [SMILES: COCC(c1ccnc(NC(=O)[C@@H](NC(=O)c2nonc2C2CC2)C2CCC(F)(F)CC2)c1)[C@@H]1C[C@@H](C(F)(F)F)NC1=O]

To a mixture of (2S)-2-amino-2-(4,4-difluorocyclohexyl)-N-(4-((R or S)-2-methoxy-1-((3S,5S)-2-oxo-5-(trifluoromethyl)pyrrolidin-3-yl)ethyl)pyridin-2-yl)acetamide (Intermediate 51-1-DS1, 7 mg), 4-cyclopropyl-1,2,5-oxadiazole-3-carboxylic acid (3 mg) and DCM (1 ml) DIPEA (10 μl) and T3P (30 μl) were added. After stirring for 1 h the mixture was concentrated in vacuo and the residue was purified by preparative RP HPLC (Agilent Prep C18, 30 mm×100 mm, 5 μm; 40 ml/min, from 97% H₂O/3% ACN to 10% H₂O/90% ACN in 12.5 min). The pure product containing fractions were combined, the ACN was partly removed and the residue freeze dried to yield 4 mg of the title compound. LC/MS: m/z=615.3 [M+H]⁺; rt: 2.30 min (LC/MS-method A). ¹H NMR (400 MHz, DMSO-d₆) δ ppm: 10.74 (s, 1H), 9.29 (d, J=7.7 Hz, 1H), 8.61 (s, 1H), 8.25 (d, J=5.1 Hz, 1H), 7.99 (s, 1H), 7.06 (d, J=5.1 Hz, 1H), 4.79-4.70 (m, 1H), 4.11-4.00 (m, 1H), 3.86 (dd, J=9.6, 5.7 Hz, 1H), 3.75-3.66 (m, 1H), 3.31-3.23 (m, 1H, partially overlapping water), 3.22 (s, 3H), 2.89-2.80 (m, 1H), 2.28 (s, 1H), 2.12-1.65 (m, 9H), 1.57-1.30 (m, 2H), 1.17-1.09 (m, 2H), 1.01-0.94 (m, 2H).

### Example 185: 4-Cyclopropyl-N-((1S)-1-(4,4-difluorocyclohexyl)-2-((4-((R or S)-2-methoxy-1-((3R,5S)-2-oxo-5-(trifluoromethyl)pyrrolidin-3-yl)ethyl)pyridin-2-yl)amino)-2-oxoethyl)-1,2,5-oxadiazole-3-carboxamide—DS2

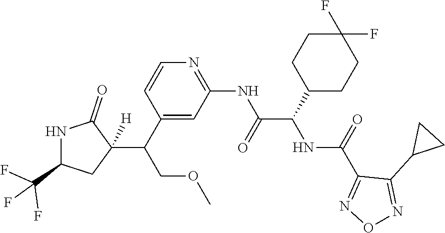 [SMILES: COCC(c1ccnc(NC(=O)[C@@H](NC(=O)c2nonc2C2CC2)C2CCC(F)(F)CC2)c1)[C@H]1C[C@@H](C(F)(F)F)NC1=O]

Using (2S)-2-amino-2-(4,4-difluorocyclohexyl)-N-(4-((R or S)-2-methoxy-1-((3R,5S)-2-oxo-5-(trifluoromethyl)pyrrolidin-3-yl)ethyl)pyridin-2-yl)acetamide (Intermediate 51-2-DS2, 12 mg) as starting material, analogously to Example 184, 8 mg of the title compound were prepared. LC/MS: m/z=615.3 [M+H]⁺; rt: 2.23 min (LC/MS-method A). ¹H NMR (400 MHz, DMSO-d₆) δ ppm: 10.71 (s, 1H), 9.28 (d, J=8.1 Hz, 1H), 8.55 (s, 1H), 8.24 (d, J=5.2 Hz, 1H), 7.97 br (s, 1H), 7.02 (dd, J=5.2, 1.5 Hz, 1H), 4.78-4.71 (m, 1H), 4.22-4.13 (m, 1H), 3.84 (dd, J=9.6, 5.6 Hz, 1H), 3.70 (dd, J=9.6, 7.8 Hz, 1H), 3.31-3.23 (m, 1H, partially overlapping water), 3.22 (s, 3H), 2.92-2.84 (m, 1H), 2.32-2.17 (m, 2H), 2.12-1.98 (m, 3H), 1.91-1.64 (m, 4H), 1.56-1.31 (m, 3H), 1.16-1.09 (m, 2H), 1.01-0.95 (m, 2H).

### Example 186: 4-Cyclopropyl-N-((1S)-1-(4,4-difluorocyclohexyl)-2-((4-((R or S)-2-methoxy-1-((3R,5S)-2-oxo-5-(trifluoromethyl)pyrrolidin-3-yl)ethyl)pyridin-2-yl)amino)-2-oxoethyl)-1,2,5-oxadiazole-3-carboxamide—DS1

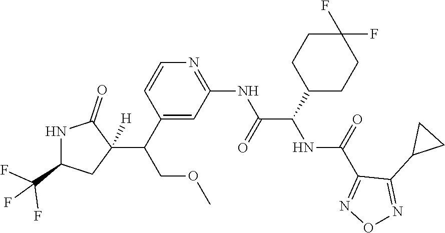 [SMILES: COCC(c1ccnc(NC(=O)[C@@H](NC(=O)c2nonc2C2CC2)C2CCC(F)(F)CC2)c1)[C@H]1C[C@@H](C(F)(F)F)NC1=O]

Using (2S)-2-amino-2-(4,4-difluorocyclohexyl)-N-(4-((R or S)-2-methoxy-1-((3R,5S)-2-oxo-5-(trifluoromethyl)pyrrolidin-3-yl)ethyl)pyridin-2-yl)acetamide (Intermediate 51-2-DS1, 12 mg) as starting material, analogously to Example 184, 9 mg of the title compound were prepared. LC/MS: m/z=615.3 [M+H]⁺; rt: 2.25 min (LC/MS-method A). ¹H NMR (400 MHz, DMSO-d₆) δ ppm: 10.74 (s, 1H), 9.29 (d, J=8.1 Hz, 1H), 8.54 (s, 1H), 8.24 (d, J=5.2 Hz, 1H), 8.03 (br s, 1H), 7.09 (dd, J=5.2, 1.5 Hz, 1H), 4.78-4.71 (m, 1H), 4.28-4.17 (m, 1H), 3.72 (dd, J=9.7, 7.9 Hz, 1H), 3.58 (dd, J=9.7, 5.6 Hz, 1H), 3.27-3.21 (m, 1H), 3.18 (s, 3H), 2.90-2.82 (m, 1H), 2.38-2.23 (m, 2H), 2.12-1.98 (m, 3H), 1.93-1.64 (m, 5H), 1.56-1.31 (m, 2H), 1.16-1.09 (m, 2H), 1.01-0.95 (m, 2H).

### Example 187: N—((S)-1-(4,4-Difluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-4-ethyl-1,2,5-oxadiazole-3-carboxamide

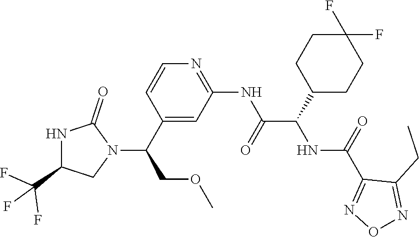 [SMILES: CCc1nonc1C(=O)N[C@H](C(=O)Nc1cc([C@@H](COC)N2C[C@@H](C(F)(F)F)NC2=O)ccn1)C1CCC(F)(F)CC1]

To a mixture of (S)-2-amino-2-(4,4-difluorocyclohexyl)-N-(4-((S)-2-methoxy-1-((S)-2-oxo-4-(trifluoromethyl)imidazolidin-1-yl)ethyl)pyridin-2-yl)acetamide hydrochloride (Intermediate 52, 20 mg), 4-ethyl-1,2,5-oxadiazole-3-carboxylic acid (6 mg) and DCM (1 ml) DIPEA (34 μl) and T3P (69 μl) were added. After stirring for 20 min the mixture was concentrated in vacuo and the residue was purified by preparative RP HPLC (Agilent Prep C18, 21.2 mm×250 mm, 10 μm; 50 ml/min, from 90% H₂O/10% ACN to 10% H₂O/90% ACN in 12.5 min). The pure product containing fractions were combined, the ACN was partly removed and the residue freeze dried to yield 14 mg of the title compound. LC/MS: m/z=604.2 [M+H]⁺; rt: 2.15 min (LC/MS-method A). ¹H NMR (400 MHz, DMSO-d₆) δ ppm: 10.79 (s, 1H), 9.25 (d, J=8.0 Hz, 1H), 8.31 (d, J=5.2 Hz, 1H), 8.02 (br s, 1H), 7.63 (d, J=2.3 Hz, 1H), 7.02 (dd, J=5.2, 1.5 Hz, 1H), 5.01 (dd, J=8.2, 5.0 Hz, 1H), 4.75-4.69 (m, 1H), 4.50-4.39 (m, 1H), 3.83 (dd, J=10.5, 8.2 Hz, 1H), 3.79-3.71 (m, 2H), 3.33-3.28 (m, 4H, overlapping water), 2.89 (q, J=7.5 Hz, 2H), 2.12-1.97 (m, 3H), 1.92-1.63 (m, 4H), 1.55-1.29 (m, 2H), 1.24 (t, J=7.5 Hz, 3H).

Using (S)-2-amino-2-(4,4-difluorocyclohexyl)-N-(4-((S)-2-methoxy-1-((S)-2-oxo-4-(trifluoromethyl)imidazolidin-1-yl)ethyl)pyridin-2-yl)acetamide hydrochloride (Intermediate 52) as starting material, analogously to Example 187 were prepared:

| Ex. | Acid | Structure | Name | Yield |
|---|---|---|---|---|
| 188 | | | N-((S)-1-(4,4- difluorocyclohexyl)-2-((4-((S)- 2-methoxy-1-((S)-2-oxo-4- (trifluoromethyl)imidazolidin-1- yl)ethyl)pyridin-2-yl)amino)-2- oxoethyl)-4- isopropylisoxazole-3- carboxamide | 6 mg, 26% |
| 189 | 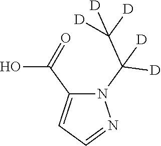 [SMILES: [2H]C([2H])([2H])C([2H])([2H])n1nccc1C(=O)O] | 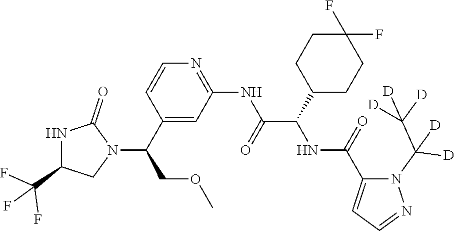 [SMILES: [2H]C([2H])([2H])C([2H])([2H])n1nccc1C(=O)N[C@H](C(=O)Nc1cc([C@@H](COC)N2C[C@@H](C(F)(F)F)NC2=O)ccn1)C1CCC(F)(F)CC1] | N-((S)-1-(4,4- difluorocyclohexyl)-2-((4-((S)- 2-methoxy-1-((S)-2-oxo-4- (trifluoromethyl)imidazolidin-1- yl)ethyl)pyridin-2-yl)amino)-2- oxoethyl)-1-(ethyl-d5)-1H- pyrazole-5-carboxamide | 9 mg, 40% |
| 190 | 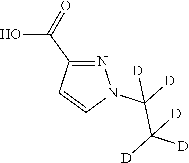 [SMILES: [2H]C([2H])([2H])C([2H])([2H])n1ccc(C(=O)O)n1] | 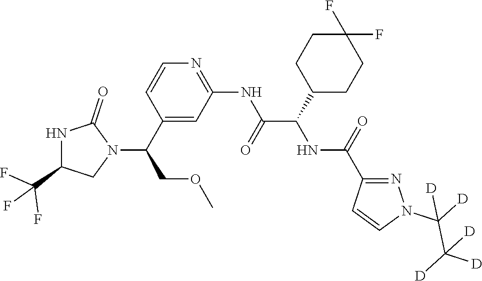 [SMILES: [2H]C([2H])([2H])C([2H])([2H])n1ccc(C(=O)N[C@H](C(=O)Nc2cc([C@@H](COC)N3C[C@@H](C(F)(F)F)NC3=O)ccn2)C2CCC(F)(F)CC2)n1] | N-((S)-1-(4,4- difluorocyclohexyl)-2-((4-((S)- 2-methoxy-1-((S)-2-oxo-4- (trifluoromethyl)imidazolidin-1- yl)ethyl)pyridin-2-yl)amino)-2- oxoethyl)-1-(ethyl-d5)-1H- pyrazole-3-carboxamide | 9 mg, 38% |
| 191 | 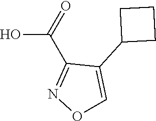 [SMILES: O=C(O)c1nocc1C1CCC1] | 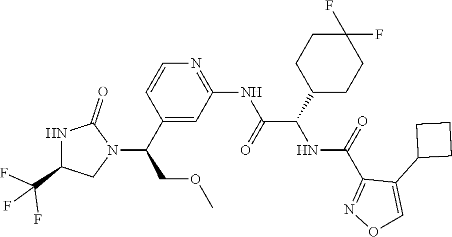 [SMILES: COC[C@H](c1ccnc(NC(=O)[C@@H](NC(=O)c2nocc2C2CCC2)C2CCC(F)(F)CC2)c1)N1C[C@@H](C(F)(F)F)NC1=O] | 4-cyclobutyl-N-((S)-1-(4,4- difluorocyclohexyl)-2-((4-((S)- 2-methoxy-1-((S)-2-oxo-4- (trifluoromethyl)imidazolidin-1- yl)ethyl)pyridin-2-yl)amino)-2- oxoethyl)isoxazole-3- carboxamide | 12 mg, 48% |
| 192 | 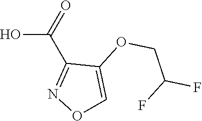 [SMILES: O=C(O)c1nocc1OCC(F)F] | 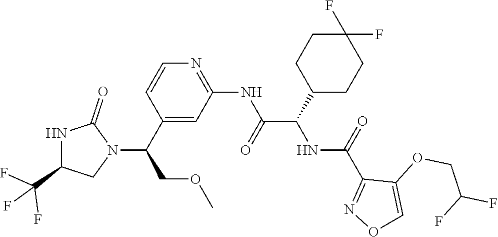 [SMILES: COC[C@H](c1ccnc(NC(=O)[C@@H](NC(=O)c2nocc2OCC(F)F)C2CCC(F)(F)CC2)c1)N1C[C@@H](C(F)(F)F)NC1=O] | N-((S)-1-(4,4- difluorocyclohexyl)-2-((4-((S)- 2-methoxy-1-((S)-2-oxo-4- (trifluoromethyl)imidazolidin-1- yl)ethyl)pyridin-2-yl)amino)-2- oxoethyl)-4-(2,2- difluoroethoxy)isoxazole-3- carboxamide | 13 mg, 52% |
| 193 | 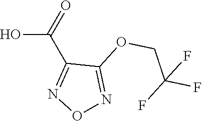 [SMILES: O=C(O)c1nonc1OCC(F)(F)F] | 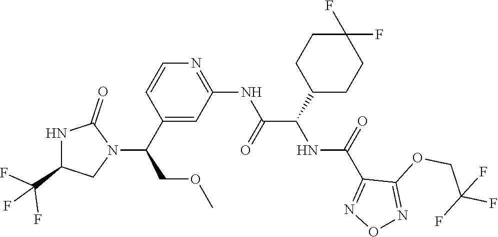 [SMILES: COC[C@H](c1ccnc(NC(=O)[C@@H](NC(=O)c2nonc2OCC(F)(F)F)C2CCC(F)(F)CC2)c1)N1C[C@@H](C(F)(F)F)NC1=O] | N-((S)-1-(4,4- difluorocyclohexyl)-2-((4-((S)- 2-methoxy-1-((S)-2-oxo-4- (trifluoromethyl)imidazolidin-1- yl)ethyl)pyridin-2-yl)amino)-2- oxoethyl)-4-(2,2,2- trifluoroethoxy)-1,2,5- oxadiazole-3-carboxamide | 19 mg, 41% |
| 194 | 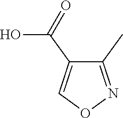 | 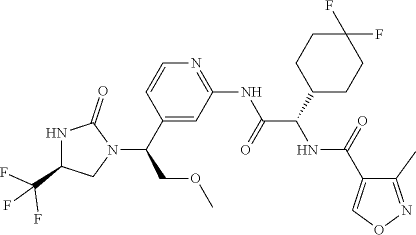 [SMILES: COC[C@H](c1ccnc(NC(=O)[C@@H](NC(=O)c2conc2C)C2CCC(F)(F)CC2)c1)N1C[C@@H](C(F)(F)F)NC1=O] | N-((S)-1-(4,4- difluorocyclohexyl)-2-((4-((S)- 2-methoxy-1-((S)-2-oxo-4- (trifluoromethyl)imidazolidin-1- yl)ethyl)pyridin-2-yl)amino)-2- oxoethyl)-3-methylisoxazole- 4-carboxamide | 9 mg, 38% |
| 195 | 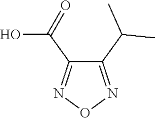 | 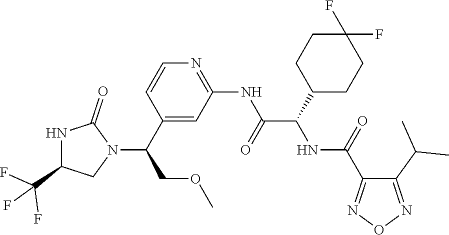 [SMILES: COC[C@H](c1ccnc(NC(=O)[C@@H](NC(=O)c2nonc2C(C)C)C2CCC(F)(F)CC2)c1)N1C[C@@H](C(F)(F)F)NC1=O] | N-((S)-1-(4,4- difluorocyclohexyl)-2-((4-((S)- 2-methoxy-1-((S)-2-oxo-4- (trifluoromethyl)imidazolidin-1- yl)ethyl)pyridin-2-yl)amino)-2- oxoethyl)-4-isopropyl-1,2,5- oxadiazole-3-carboxamide | 14 mg, 62% |
| 196 | 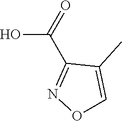 | 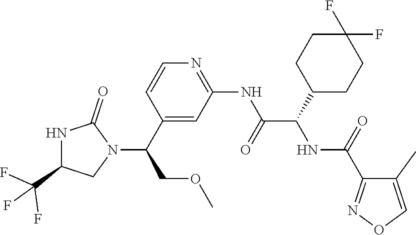 [SMILES: COC[C@H](c1ccnc(NC(=O)[C@@H](NC(=O)c2nocc2C)C2CCC(F)(F)CC2)c1)N1C[C@@H](C(F)(F)F)NC1=O] | N-((S)-1-(4,4- difluorocyclohexyl)-2-((4-((S)- 2-methoxy-1-((S)-2-oxo-4- (trifluoromethyl)imidazolidin-1- yl)ethyl)pyridin-2-yl)amino)-2- oxoethyl)-4-methylisoxazole- 3-carboxamide | 16 mg, 68% |
| 197 | 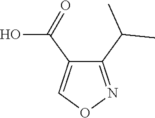 | 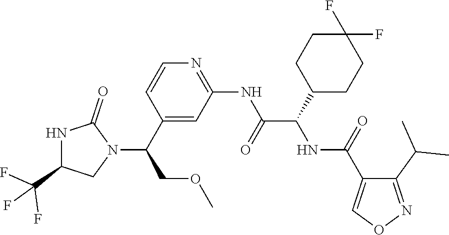 [SMILES: COC[C@H](c1ccnc(NC(=O)[C@@H](NC(=O)c2conc2C(C)C)C2CCC(F)(F)CC2)c1)N1C[C@@H](C(F)(F)F)NC1=O] | N-((S)-1-(4,4- difluorocyclohexyl)-2-((4-((S)- 2-methoxy-1-((S)-2-oxo-4- (trifluoromethyl)imidazolidin-1- yl)ethyl)pyridin-2-yl)amino)-2- oxoethyl)-3- isopropylisoxazole-4- carboxamide | 14 mg, 60% |
| 198 | 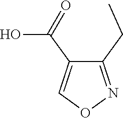 | 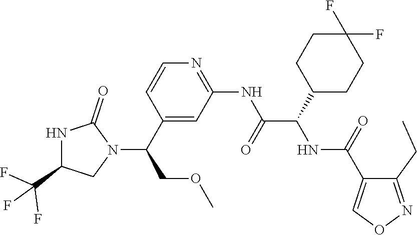 [SMILES: CCc1nocc1C(=O)N[C@H](C(=O)Nc1cc([C@@H](COC)N2C[C@@H](C(F)(F)F)NC2=O)ccn1)C1CCC(F)(F)CC1] | N-((S)-1-(4,4- difluorocyclohexyl)-2-((4-((S)- 2-methoxy-1-((S)-2-oxo-4- (trifluoromethyl)imidazolidin-1- yl)ethyl)pyridin-2-yl)amino)-2- oxoethyl)-3-ethylisoxazole-4- carboxamide | 15 mg, 65% |
| 199 | 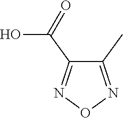 | 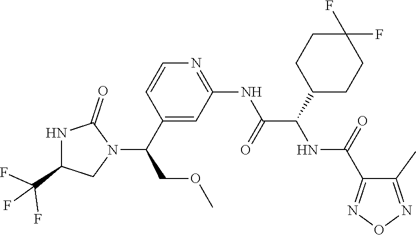 [SMILES: COC[C@H](c1ccnc(NC(=O)[C@@H](NC(=O)c2nonc2C)C2CCC(F)(F)CC2)c1)N1C[C@@H](C(F)(F)F)NC1=O] | N-((S)-1-(4,4- difluorocyclohexyl)-2-((4-((S)- 2-methoxy-1-((S)-2-oxo-4- (trifluoromethyl)imidazolidin-1- yl)ethyl)pyridin-2-yl)amino)-2- oxoethyl)-4-methyl-1,2,5- oxadiazole-3-carboxamide | 15 mg, 68% |
| 200 | 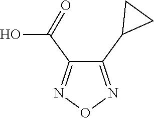 [SMILES: O=C(O)c1nonc1C1CC1] | 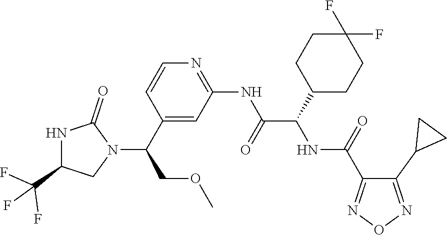 [SMILES: COC[C@H](c1ccnc(NC(=O)[C@@H](NC(=O)c2nonc2C2CC2)C2CCC(F)(F)CC2)c1)N1C[C@@H](C(F)(F)F)NC1=O] | 4-cyclopropyl-N-((S)-1-(4,4- difluorocyclohexyl)-2-((4-((S)- 2-methoxy-1-((S)-2-oxo-4- (trifluoromethyl)imidazolidin-1- yl)ethyl)pyridin-2-yl)amino)-2- oxoethyl)-1,2,5-oxadiazole-3- carboxamide | 14 mg, 63% |
| 201 | 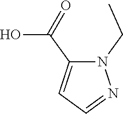 | 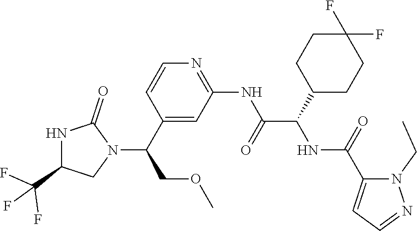 [SMILES: CCn1nccc1C(=O)N[C@H](C(=O)Nc1cc([C@@H](COC)N2C[C@@H](C(F)(F)F)NC2=O)ccn1)C1CCC(F)(F)CC1] | N-((S)-1-(4,4- difluorocyclohexyl)-2-((4-((S)- 2-methoxy-1-((S)-2-oxo-4- (trifluoromethyl)imidazolidin-1- yl)ethyl)pyridin-2-yl)amino)-2- oxoethyl)-1-ethyl-1H-pyrazole- 5-carboxamide | 15 mg, 63% |
| 202 | 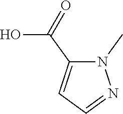 | 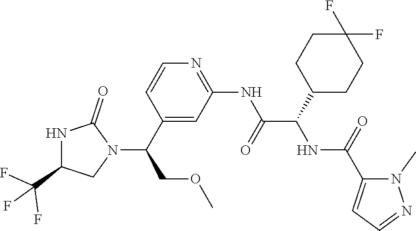 [SMILES: COC[C@H](c1ccnc(NC(=O)[C@@H](NC(=O)c2ccnn2C)C2CCC(F)(F)CC2)c1)N1C[C@@H](C(F)(F)F)NC1=O] | N-((S)-1-(4,4- difluorocyclohexyl)-2-((4-((S)- 2-methoxy-1-((S)-2-oxo-4- (trifluoromethyl)imidazolidin-1- yl)ethyl)pyridin-2-yl)amino)-2- oxoethyl)-1-methyl-1H- pyrazole-5-carboxamide | 12 mg, 55% |
| 203 | 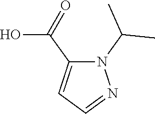 | 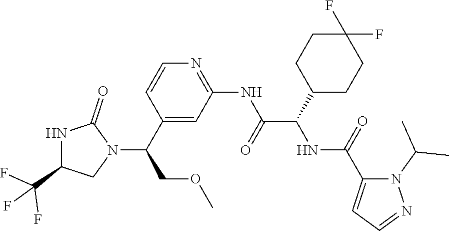 [SMILES: COC[C@H](c1ccnc(NC(=O)[C@@H](NC(=O)c2ccnn2C(C)C)C2CCC(F)(F)CC2)c1)N1C[C@@H](C(F)(F)F)NC1=O] | N-((S)-1-(4,4- difluorocyclohexyl)-2-((4-((S)- 2-methoxy-1-((S)-2-oxo-4- (trifluoromethyl)imidazolidin-1- yl)ethyl)pyridin-2-yl)amino)-2- oxoethyl)-1-isopropyl-1H- pyrazole-5-carboxamide | 13 mg, 55% |
| 204 | 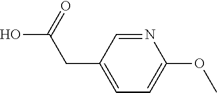 | 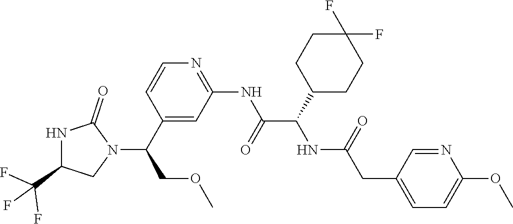 [SMILES: COC[C@H](c1ccnc(NC(=O)[C@@H](NC(=O)Cc2ccc(OC)nc2)C2CCC(F)(F)CC2)c1)N1C[C@@H](C(F)(F)F)NC1=O] | (S)-2-(4,4-difluorocyclohexyl)- N-(4-((S)-2-methoxy-1-((S)-2- oxo-4- (trifluoromethyl)imidazolidin-1- yl)ethyl)pyridin-2-yl)-2-(2-(6- methoxypyridin-3- yl)acetamido)acetamide | 10 mg, 57% |
| 205 | 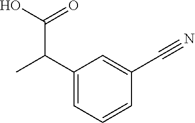 | 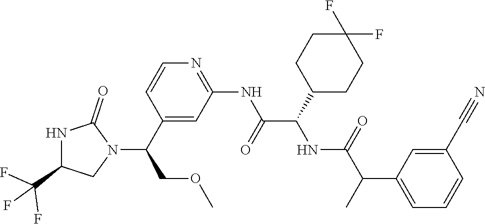 [SMILES: COC[C@H](c1ccnc(NC(=O)[C@@H](NC(=O)C(C)c2cccc(C#N)c2)C2CCC(F)(F)CC2)c1)N1C[C@@H](C(F)(F)F)NC1=O] | 2-(3-cyanophenyl)-N-((S)-1- (4,4-difluorocyclohexyl)-2-((4- ((S)-2-methoxy-1-((S)-2-oxo- 4-(trifluoromethyl)imidazolidin- 1-yl)ethyl)pyridin-2- yl)amino)-2-oxoethyl) propanamide | 10 mg, 58% |
| 206 | 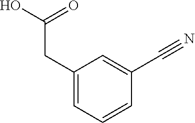 [SMILES: N#Cc1cccc(CC(=O)O)c1] | 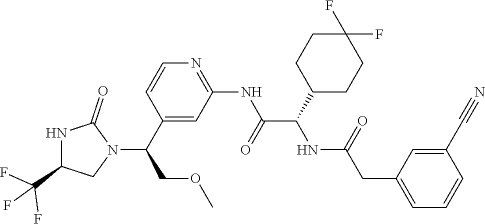 [SMILES: COC[C@H](c1ccnc(NC(=O)[C@@H](NC(=O)Cc2cccc(C#N)c2)C2CCC(F)(F)CC2)c1)N1C[C@@H](C(F)(F)F)NC1=O] | (S)-2-(2-(3- cyanophenyl)acetamido)-2- (4,4-difluorocyclohexyl)-N-(4- ((S)-2-methoxy-1-((S)-2-oxo- 4-(trifluoromethyl)imidazolidin- 1-yl)ethyl)pyridin-2- yl)acetamide | 12 mg, 70% |

### Example 188

LC/MS: m/z=617.1 [M+H]⁺; rt: 2.38 min (LC/MS-method A). ¹H NMR (400 MHz, DMSO-de) δ ppm: 10.73 (s, 1H), 8.87 (d, J=0.9 Hz, 1H), 8.79 (d, J=8.2 Hz, 1H), 8.31 (d, J=5.2 Hz, 1H), 8.02 (br s, 1H), 7.63 (d, J=2.3 Hz, 1H), 7.01 (dd, J=5.2, 1.5 Hz, 1H), 5.01 (dd, J=8.2, 5.1 Hz, 1H), 4.72-4.65 (m, 1H), 4.50-4.38 (m, 1H), 3.83 (dd, J=10.5, 8.2 Hz, 1H), 3.79-3.71 (m, 2H), 3.33-3.28 (m, 4H, overlapping water), 3.09-2.98 (m, 1H), 2.11-1.96 (m, 3H), 1.92-1.62 (m, 4H), 1.53-1.28 (m, 2H), 1.19-1.12 (m, 6H).

### Example 189

LC/MS: m/z=607.2 [M+H]⁺; rt: 2.06 min (LC/MS-method A). ¹H NMR (400 MHz, DMSO-de) δ ppm: 10.69 (s, 1H), 8.56 (d, J=7.9 Hz, 1H), 8.30 (d, J=5.2 Hz, 1H), 8.02 (br s, 1H), 7.62 (d, J=2.3 Hz, 1H), 7.48 (d, J=2.0 Hz, 1H), 7.02-6.98 (m, 2H), 5.01 (dd, J=8.2, 5.1 Hz, 1H), 4.66-4.60 (m, 1H), 4.49-4.38 (m, 1H), 3.83 (dd, J=10.5, 8.2 Hz, 1H), 3.78-3.70 (m, 2H), 3.32-3.27 (m, 4H, overlapping water), 2.13-1.63 (m, 7H), 1.55-1.28 (m, 2H).

### Example 190

LC/MS: m/z=607.2 [M+H]⁺; rt: 2.06 min (LC/MS-method A). ¹H NMR (400 MHz, DMSO-de) δ ppm: 10.79 (s, 1H), 8.30 (d, J=5.2 Hz, 1H), 8.02 (br s, 1H), 7.88 (d, J=8.9 Hz, 1H), 7.85 (d, J=2.3 Hz, 1H), 7.63 (d, J=2.3 Hz, 1H), 7.01 (dd, J=5.2, 1.5 Hz, 1H), 6.65 (d, J=2.3 Hz, 1H), 5.01 (dd, J=8.2, 5.1 Hz, 1H), 4.77-4.70 (m, 1H), 4.49-4.39 (m, 1H), 3.83 (dd, J=10.5, 8.2 Hz, 1H), 3.78-3.71 (m, 2H), 3.33-3.27 (m, 4H, overlapping water), 2.09-1.95 (m, 3H), 1.89-1.66 (m, 4H), 1.49-1.22 (m, 2H).

### Example 191

LC/MS: m/z=629.1 [M+H]⁺; rt: 2.31 min (LC/MS-method A). ¹H NMR (400 MHz, DMSO-de) δ ppm: 10.72 (s, 1H), 8.98 (d, J=1.0 Hz, 1H), 8.71 (d, J=8.2 Hz, 1H), 8.31 (d, J=5.2 Hz, 1H), 8.02 (br s, 1H), 7.63 (d, J=2.3 Hz, 1H), 7.01 (dd, J=5.2, 1.5 Hz, 1H), 5.01 (dd, J=8.2, 5.1 Hz, 1H), 4.70-4.64 (m, 1H), 4.50-4.38 (m, 1H), 3.83 (dd, J=10.5, 8.2 Hz, 1H), 3.78-3.71 (m, 2H), 3.57-3.46 (m, 1H), 3.32-3.27 (m, 4H, overlapping water), 2.30-2.20 (m, 2H), 2.11-1.63 (m, 11H), 1.52-1.25 (m, 2H).

### Example 192

LC/MS: m/z=655.2 [M+H]⁺; rt: 1.94 min (LC/MS-method A). ¹H NMR (400 MHz, DMSO-de) δ ppm: 10.77 (s, 1H), 9.06 (s, 1H), 8.67 (d, J=8.2 Hz, 1H), 8.31 (d, J=5.2 Hz, 1H), 8.02 (br s, 1H), 7.63 (d, J=2.3 Hz, 1H), 7.01 (br d, J=5.2 Hz, 1H), 6.36 (tt, J=54.1, 3.5 Hz, 1H), 5.01 (dd, J=8.3, 5.1 Hz, 1H), 4.75-4.68 (m, 1H), 4.49-4.38 (m, 1H), 4.32 (td, J=14.5, 3.5 Hz, 2H), 3.83 (dd, J=10.4, 8.3 Hz, 1H), 3.78-3.71 (m, 2H), 3.33-3.27 (m, 4H, overlapping water), 2.11-1.63 (m, 7H), 1.53-1.27 (m, 2H).

### Example 193

LC/MS: m/z=674.2 [M+H]⁺; rt: 2.16 min (LC/MS-method A). ¹H NMR (400 MHz, DMSO-de) δ ppm: 10.83 (s, 1H), 9.22 (d, J=8.2 Hz, 1H), 8.31 (d, J=5.2 Hz, 1H), 8.02 (br s, 1H), 7.63 (d, J=2.3 Hz, 1H), 7.01 (dd, J=5.2, 1.5 Hz, 1H), 5.20-5.09 (m, 2H), 5.01 (dd, J=8.1, 5.1 Hz, 1H), 4.79-4.72 (m, 1H), 4.50-4.38 (m, 1H), 3.83 (dd, J=10.6, 8.1 Hz, 1H), 3.78-3.71 (m, 2H), 3.33-3.27 (m, 4H, overlapping water), 2.11-1.93 (m, 3H), 1.91-1.63 (m, 4H), 1.55-1.28 (m, 2H).

### Example 194

LC/MS: m/z=589.2 [M+H]⁺; rt: 1.84 min (LC/MS-method A). ¹H NMR (400 MHz, DMSO-de) δ ppm: 10.72 (s, 1H), 9.42 (s, 1H), 8.50 (d, J=8.1 Hz, 1H), 8.30 (d, J=5.3 Hz, 1H), 8.02 (br s, 1H), 7.62 (d, J=2.3 Hz, 1H), 7.01 (br d, J=5.3 Hz, 1H), 5.00 (dd, J=8.1, 5.0 Hz, 1H), 4.70-4.64 (m, 1H), 4.49-4.39 (m, 1H), 3.83 (dd, J=10.5, 8.1 Hz, 1H), 3.78-3.71 (m, 2H), 3.33-3.26 (m, 4H, overlapping water), 2.36 (s, 3H), 2.13-1.61 (m, 7H), 1.55-1.29 (m, 2H).

### Example 195

LC/MS: m/z=618.2 [M+H]⁺; rt: 2.28 min (LC/MS-method A). ¹H NMR (400 MHz, DMSO-de) δ ppm: 10.80 (s, 1H), 9.33 (d, J=8.0 Hz, 1H), 8.31 (d, J=5.2 Hz, 1H), 8.02 (br s, 1H), 7.63 (d, J=2.3 Hz, 1H), 7.02 (dd, J=5.2, 1.6 Hz, 1H), 5.01 (dd, J=8.2, 5.0 Hz, 1H), 4.76-4.71 (m, 1H), 4.48-4.41 (m, 1H), 3.83 (dd, J=10.6, 8.2 Hz, 1H), 3.78-3.72 (m, 2H), 3.37-3.28 (m, 5H, overlapping water), 2.10-1.98 (m, 3H), 1.89-1.65 (m, 4H), 1.53-1.44 (m, 1H), 1.41-1.33 (m, 1H), 1.28 (d, J=6.9 Hz, 6H).

### Example 196

LC/MS: m/z=589.2 [M+H]⁺; rt: 1.95 min (LC/MS-method A). ¹H NMR (400 MHz, DMSO-de) δ ppm: 10.75 (s, 1H), 8.84 (q, J=1.1 Hz, 1H), 8.69 (d, J=8.2 Hz, 1H), 8.31 (d, J=5.2 Hz, 1H), 8.02 (br s, 1H), 7.63 (d, J=2.3 Hz, 1H), 7.01 (dd, J=5.2, 1.5 Hz, 1H), 5.01 (dd, J=8.2, 5.0 Hz, 1H), 4.71-4.64 (m, 1H), 4.50-4.39 (m, 1H), 3.83 (dd, J=10.5, 8.2 Hz, 1H), 3.78-3.71 (m, 2H), 3.35-3.27 (m, 4H, overlapping water), 2.12-1.96 (m, 6H), 1.91-1.63 (m, 4H), 1.53-1.26 (m, 2H).

### Example 197

LC/MS: m/z=617.2 [M+H]⁺; rt: 2.07 min (LC/MS-method A). ¹H NMR (400 MHz, DMSO-de) δ ppm: 10.70 (s, 1H), 9.35 (s, 1H), 8.55 (d, J=8.1 Hz, 1H), 8.30 (d, J=5.3 Hz, 1H), 8.02 (br s, 1H), 7.62 (d, J=2.3 Hz, 1H), 7.00 (dd, J=5.3, 1.5 Hz, 1H), 5.01 (dd, J=8.2, 5.0 Hz, 1H), 4.70-4.63 (m, 1H), 4.49-4.39 (m, 1H), 3.83 (dd, J=10.5, 8.2 Hz, 1H), 3.78-3.71 (m, 2H), 3.41 (hept, J=6.9 Hz, 1H), 3.32-3.26 (m, 4H, overlapping water), 2.13-1.62 (m, 7H), 1.54-1.30 (m, 2H), 1.23 (d, J=6.9 Hz, 3H), 1.21 (d, J=6.9 Hz, 3H).

### Example 198

LC/MS: m/z=603.3 [M+H]⁺; rt: 1.96 min (LC/MS-method A). ¹H NMR (400 MHz, DMSO-de) δ ppm: 10.71 (s, 1H), 9.40 (s, 1H), 8.52 (d, J=8.1 Hz, 1H), 8.30 (d, J=5.2 Hz, 1H), 8.02 (br s, 1H), 7.62 (d, J=2.3 Hz, 1H), 7.00 (dd, J=5.2, 1.5 Hz, 1H), 5.01 (dd, J=8.2, 5.0 Hz, 1H), 4.70-4.64 (m, 1H), 4.49-4.39 (m, 1H), 3.83 (dd, J=10.6, 8.2 Hz, 1H), 3.78-3.71 (m, 2H), 3.33-3.26 (m, 4H, overlapping water), 2.83 (q, J=7.5 Hz, 2H), 2.13-1.62 (m, 7H), 1.55-1.29 (m, 2H), 1.16 (t, J=7.5 Hz, 3H).

### Example 199

LC/MS: m/z=590.1 [M+H]⁺; rt: 2.01 min (LC/MS-method A). ¹H NMR (400 MHz, DMSO-d6) δ ppm: 10.79 (s, 1H), 9.22 (d, J=8.0 Hz, 1H), 8.31 (d, J=5.2 Hz, 1H), 8.02 (br s, 1H), 7.63 (d, J=2.0 Hz, 1H), 7.02 (br d, J=5.2 Hz, 1H), 5.03-4.98 (m, 1H), 4.74-4.68 (m, 1H), 4.49-4.39 (m, 1H), 3.86-3.80 (m, 1H), 3.78-3.71 (m, 2H), 3.34-3.28 (m, 4H, overlapping water), 2.47 (s, 3H), 2.11-1.96 (m, 3H), 1.92-1.65 (m, 4H), 1.55-1.29 (m, 2H).

### Example 200

LC/MS: m/z=616.1 [M+H]⁺; rt: 2.19 min (LC/MS-method A). ¹H NMR (400 MHz, DMSO-de) δ ppm: 10.80 (s, 1H), 9.30 (d, J=7.8 Hz, 1H), 8.31 (d, J=5.2 Hz, 1H), 8.02 (s, 1H), 7.63 (s, 1H), 7.02 (d, J=5.2 Hz, 1H), 5.04-4.98 (m, 1H), 4.77-4.70 (m, 1H), 4.48-4.40 (m, 1H), 3.87-3.80 (m, 1H), 3.79-3.71 (m, 2H), 3.43-3.19 (m, 4H, overlapping water), 2.31-2.23 (m, 1H), 2.13-1.97 (m, 3H), 1.92-1.65 (m, 4H), 1.56-1.31 (m, 2H), 1.16-1.09 (m, 2H), 1.01-0.94 (m, 2H).

### Example 201

LC/MS: m/z=602.2 [M+H]⁺; rt: 1.89 min (LC/MS-method A). ¹H NMR (400 MHz, DMSO-de) δ ppm: 10.69 (s, 1H), 8.56 (d, J=8.0 Hz, 1H), 8.30 (d, J=5.3 Hz, 1H), 8.02 (br s, 1H), 7.62 (d, J=2.3 Hz, 1H), 7.48 (d, J=2.0 Hz, 1H), 7.02-6.98 (m, 2H), 5.01 (dd, J=8.2, 5.1 Hz, 1H), 4.67-4.59 (m, 1H), 4.50-4.39 (m, 3H), 3.83 (dd, J=10.5, 8.2 Hz, 1H), 3.78-3.71 (m, 2H), 3.40-3.27 (m, 4H, overlapping water), 2.14-1.62 (m, 7H), 1.56-1.23 (m, 5H).

### Example 202

LC/MS: m/z=588.1 [M+H]⁺; rt: 1.78 min (LC/MS-method A). ¹H NMR (400 MHz, DMSO-de) δ ppm: 10.70 (s, 1H), 8.56 (d, J=8.0 Hz, 1H), 8.30 (d, J=5.1 Hz, 1H), 8.02 (br s, 1H), 7.62 (d, J=2.2 Hz, 1H), 7.47 (d, J=2.1 Hz, 1H), 7.03 (d, J=2.1 Hz, 1H), 7.00 (dd, J=5.1, 1.4 Hz, 1H), 5.00 (dd, J=8.2, 5.1 Hz, 1H), 4.66-4.60 (m, 1H), 4.49-4.39 (m, 1H), 4.02 (s, 3H), 3.83 (dd, J=10.6, 8.2 Hz, 1H), 3.78-3.71 (m, 2H), 3.34-3.27 (m, 4H, overlapping water), 2.13-1.94 (m, 3H), 1.94-1.60 (m, 4H), 1.56-1.41 (m, 1H), 1.41-1.27 (m, 1H).

### Example 203

LC/MS: m/z=616.2 [M+H]⁺; rt: 2.00 min (LC/MS-method A). ¹H NMR (400 MHz, DMSO-de) δ ppm: 10.69 (s, 1H), 8.55 (d, J=8.0 Hz, 1H), 8.30 (d, J=5.3 Hz, 1H), 8.02 (br s, 1H), 7.62 (d, J=2.3 Hz, 1H), 7.50 (d, J=2.0 Hz, 1H), 7.01 (dd, J=5.3, 1.5 Hz, 1H), 6.93 (d, J=2.0 Hz, 1H), 5.36 (hept, J=6.7 Hz, 1H), 5.01 (dd, J=8.2, 5.0 Hz, 1H), 4.65-4.58 (m, 1H), 4.49-4.38 (m, 1H), 3.83 (dd, J=10.5, 8.2 Hz, 1H), 3.78-3.71 (m, 2H), 3.33-3.28 (m, 4H, overlapping water), 2.14-1.62 (m, 7H), 1.35 (dd, J=9.4, 6.6 Hz, 8H).

### Example 204

LC/MS: m/z=629.2 [M+H]⁺; rt: 1.82 min (LC/MS-method A). ¹H NMR (400 MHz, DMSO-de) δ ppm: 10.62 (s, 1H), 8.37 (d, J=8.3 Hz, 1H), 8.28 (d, J=5.2 Hz, 1H), 8.02-7.99 (m, 2H), 7.63 (d, J=2.3 Hz, 1H), 7.58 (dd, J=8.5, 2.5 Hz, 1H), 6.99 (dd, J=5.2, 1.6 Hz, 1H), 6.74 (d, J=8.5 Hz, 1H), 5.01 (dd, J=8.2, 5.0 Hz, 1H), 4.59-4.52 (m, 1H), 4.50-4.39 (m, 1H), 3.87-3.79 (m, 4H), 3.78-3.70 (m, 2H), 3.53-3.41 (m, 2H), 3.34-3.26 (m, 4H, overlapping water), 2.08-1.94 (m, 2H), 1.92-1.57 (m, 5H), 1.45-1.25 (m, 2H).

### Example 205

LC/MS: m/z=637.3 [M+H]⁺; rt: 2.03 min (LC/MS-method A). ¹H NMR (400 MHz, DMSO-de) δ ppm: 10.65-10.55 (m, 1H), 8.38-8.25 (m, 2H), 8.03-7.93 (m, 1H), 7.79-7.60 (m, 4H), 7.55-7.46 (m, 1H), 7.02-6.95 (m, 1H), 5.04-4.96 (m, 1H), 4.60-4.50 (m, 1H), 4.50-4.38 (m, 1H), 3.93 (q, J=7.0 Hz, 1H), 3.88-3.68 (m, 3H), 3.34-3.23 (m, 4H, overlapping water), 2.11-1.28 (m, 11H), 1.27-1.09 (m, 1H).

### Example 206

LC/MS: m/z=623.2 [M+H]⁺; rt: 1.92 min (LC/MS-method A). ¹H NMR (400 MHz, DMSO-de) δ ppm: 10.63 (s, 1H), 8.44 (d, J=8.3 Hz, 1H), 8.29 (d, J=5.1 Hz, 1H), 8.01 (br s, 1H), 7.73-7.67 (m, 2H), 7.64-7.58 (m, 2H), 7.53-7.47 (m, 1H), 6.99 (dd, J=5.1, 1.6 Hz, 1H), 5.01 (dd, J=8.2, 5.1 Hz, 1H), 4.60-4.53 (m, 1H), 4.50-4.39 (m, 1H), 3.83 (dd, J=10.5, 8.2 Hz, 1H), 3.78-3.70 (m, 2H), 3.68-3.57 (m, 2H), 3.34-3.25 (m, 4H, overlapping water), 2.08-1.94 (m, 2H), 1.93-1.57 (m, 5H), 1.44-1.26 (m, 2H).

### Example 207: N-((1S)-1-(4,4-difluorocyclohexyl)-2-oxo-2-((4-(1-(4,4,4-trifluorobutan-amido)propyl)pyridin-2-yl)amino)ethyl)-4-ethyl-1,2,5-oxadiazole-3-carboxamide

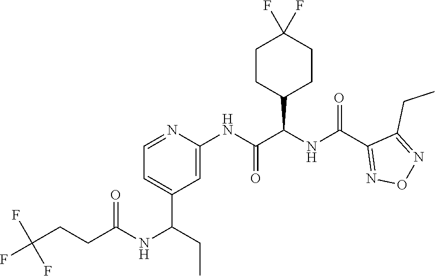 [SMILES: CCc1nonc1C(=O)N[C@@H](C(=O)Nc1cc(C(CC)NC(=O)CCC(F)(F)F)ccn1)C1CCC(F)(F)CC1]

Following the procedure described for Example 39, using 30 mg N-(1-(2-((S)-2-amino-2-(4,4-difluorocyclohexyl)acetamido)pyridin-4-yl)propyl)-4,4,4-trifluorobutanamide (Intermediate 68), 17 mg of the title compound was obtained. LC/MS: m/z=575.2 [M+H]⁺; rt: 2.31 min (LC/MS-method A). ¹H NMR (400 MHz, DMSO-de) 6 ppm: 10.77 (s, 1H), 9.21 (d, J=8.0 Hz, 1H), 8.51 (d, J=7.9 Hz, 1H), 8.26 (d, J=5.2 Hz, 1H), 8.00 (br s, 1H), 7.07 (dd, J=5.2, 1.5 Hz, 1H), 4.74-4.69 (m, 1H), 4.68-4.62 (m, 1H), 2.90 (q, J=7.5 Hz, 2H), 2.53-2.37 (m, 4H, overlapping solvent), 2.10-1.99 (m, 3H), 1.91-1.61 (m, 6H), 1.53-1.44 (m, 1H), 1.41-1.32 (m, 1H), 1.24 (t, J=7.5 Hz, 3H), 0.87 (t, J=7.3 Hz, 3H).

### Example 208: N—((S)-1-(4,4-difluorocyclohexyl)-2-oxo-2-((4-((R)-1-((S)-2-oxo-4-(trifluoromethyl)imidazolidin-1-yl)propyl)pyridin-2-yl)amino)ethyl)-4-ethylisoxazole-3-carboxamide

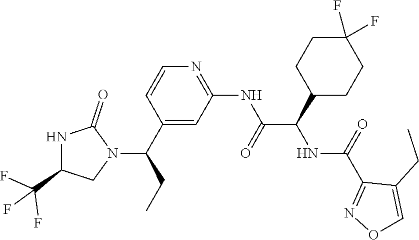 [SMILES: CCc1conc1C(=O)N[C@@H](C(=O)Nc1cc([C@@H](CC)N2C[C@@H](C(F)(F)F)NC2=O)ccn1)C1CCC(F)(F)CC1]

Was prepared by the synthesis route described for Example 158 to afford 18 mg of the title compound. LC/MS: m/z=587.2 [M+H]⁺; rt: 2.18 min (LC/MS-method A). ¹H NMR (400 MHz, DMSO-de) 6 ppm: 10.79 (s, 1H), 8.87 (t, J=0.9 Hz, 1H), 8.73 (d, J=8.2 Hz, 1H), 8.31 (d, J=5.2 Hz, 1H), 8.01 (br s, 1H), 7.55 (d, J=2.4 Hz, 1H), 7.04 (dd, J=5.2, 1.6 Hz, 1H), 4.73-4.64 (m, 2H), 4.48-4.37 (m, 1H), 3.71-3.63 (m, 1H), 3.06 (dd, J=10.0, 4.5 Hz, 1H), 2.60-2.51 (m, 2H, partially overlapping solvent), 2.11-1.63 (m, 9H), 1.54-1.26 (m, 2H), 1.12 (t, J=7.5 Hz, 3H), 0.88 (t, J=7.2 Hz, 3H).

Examples 209-213 were prepared analogously to Example 208. Examples 214-215 were prepared analogously to Example 208 using the procedure described for Example 33 in the final step.

| Ex. | Acid | Structure | Name | Yield |
|---|---|---|---|---|
| 209 | | | N-((S)-1-(4,4- | 42 mg, |
| | | | difluorocyclohexyl)-2-oxo-2- | 82% |
| | | | ((4-((R)-1-((S)-2-oxo-4- | |
| | | | (trifluoromethyl)imidazolidin-1- | |
| | | | yl)propyl)pyridin-2- | |
| | | | yl)amino)ethyl)-4-ethyl-1,2,5- | |
| | | | oxadiazole-3-carboxamide | |
| 210 | | | N-((S)-1-(4,4- | 37 mg, |
| | | | difluorocyclohexyl)-2-oxo-2- | 73% |
| | | | ((4-((R)-1-((S)-2-oxo-4- | |
| | | | (trifluoromethyl)imidazolidin-1- | |
| | | | yl)propyl)pyridin-2- | |
| | | | yl)amino)ethyl)-3- | |
| | | | ethylisoxazole-4-carboxamide | |
| 211 | | | N-((S)-1-(4,4- | 42 mg, |
| | | | difluorocyclohexyl)-2-oxo-2- | 84% |
| | | | ((4-((R)-1-((S)-2-oxo-4- | |
| | | | (trifluoromethyl)imidazolidin-1- | |
| | | | yl)propyl)pyridin-2- | |
| | | | yl)amino)ethyl)-4-methyl- | |
| | | | 1,2,5-oxadiazole-3- | |
| | | | carboxamide | |
| 212 | | | 4-cyclopropyl-N-((S)-1-(4,4- | 45 mg, |
| | | | difluorocyclohexyl)-2-oxo-2- | 87% |
| | | | ((4-((R)-1-((S)-2-oxo-4- | |
| | | | (trifluoromethyl)imidazolidin-1- | |
| | | | yl)propyl)pyridin-2- | |
| | | | yl)amino)ethyl)-1,2,5- | |
| | | | oxadiazole-3-carboxamide | |
| 213 | | | N-((S)-1-(4,4- | 42 mg, |
| | | | difluorocyclohexyl)-2-oxo-2- | 71% |
| | | | ((4-((R)-1-((S)-2-oxo-4- | |
| | | | (trifluoromethyl)imidazolidin-1- | |
| | | | yl)propyl)pyridin-2- | |
| | | | yl)amino)ethyl)-1-ethyl-1H- | |
| | | | pyrazole-5-carboxamide | |
| 214 | | | N-((S)-1-(4,4- | 45 mg, |
| | | | difluorocyclohexyl)-2-oxo-2- | 91% |
| | | | ((4-((R)-1-((S)-2-oxo-4- | |
| | | | (trifluoromethyl)imidazolidin-1- | |
| | | | yl)propyl)pyridin-2- | |
| | | | yl)amino)ethyl)-1-methyl-1H- | |
| | | | pyrazole-5-carboxamide | |
| 215 | | | N-((S)-1-(4,4- | 44 mg, |
| | | | difluorocyclohexyl)-2-oxo-2- | 85% |
| | | | ((4-((R)-1-((S)-2-oxo-4- | |
| | | | (trifluoromethyl)imidazolidin-1- | |
| | | | yl)propyl)pyridin-2- | |
| | | | yl)amino)ethyl)-1-isopropyl- | |
| | | | 1H-pyrazole-5-carboxamide | |

### Example 209

LC/MS: m/z=588.3 [M+H]⁺; rt: 2.26 min (LC/MS-method A). ¹H NMR (400 MHz, DMSO-d6) δ ppm: 10.85 (s, 1H), 9.26 (d, J=8.0 Hz, 1H), 8.31 (d, J=5.3 Hz, 1H), 8.00 (br s, 1H), 7.55 (d, J=2.4 Hz, 1H), 7.05 (dd, J=5.3, 1.6 Hz, 1H), 4.75-4.67 (m, 2H), 4.48-4.37 (m, 1H), 3.71-3.63 (m, 1H, partially overlapping water), 3.06 (dd, J=10.0, 4.5 Hz, 1H), 2.90 (q, J=7.5 Hz, 2H), 2.12-1.64 (m, 9H), 1.55-1.29 (m, 2H), 1.23 (t, J=7.5 Hz, 3H), 0.88 (t, J=7.2 Hz, 3H).

### Example 210

LC/MS: m/z=587.2 [M+H]⁺; rt: 2.04 min (LC/MS-method A). ¹H NMR (400 MHz, DMSO-d6) δ ppm: 10.78 (s, 1H), 9.41 (s, 1H), 8.53 (d, J=8.1 Hz, 1H), 8.31 (d, J=5.3 Hz, 1H), 8.01 (br s, 1H), 7.54 (d, J=2.4 Hz, 1H), 7.04 (dd, J=5.3, 1.6 Hz, 1H), 4.73-4.63 (m, 2H), 4.48-4.37 (m, 1H), 3.70-3.62 (m, 1H, partially overlapping water), 3.05 (dd, J=10.0, 4.5 Hz, 1H), 2.82 (q, J=7.5 Hz, 2H), 2.12-1.62 (m, 9H), 1.54-1.29 (m, 2H), 1.16 (t, J=7.5 Hz, 3H), 0.88 (t, J=7.2 Hz, 3H).

### Example 211

LC/MS: m/z=574.2 [M+H]⁺; rt: 2.11 min (LC/MS-method A). ¹H NMR (400 MHz, DMSO-d6) δ ppm: 10.85 (s, 1H), 9.22 (d, J=8.0 Hz, 1H), 8.31 (d, J=5.2 Hz, 1H), 8.01 (br s, 1H), 7.55 (d, J=2.4 Hz, 1H), 7.05 (dd, J=5.2, 1.6 Hz, 1H), 4.74-4.67 (m, 2H), 4.48-4.37 (m, 1H), 3.71-3.63 (m, 1H, partially overlapping water), 3.06 (dd, J=10.0, 4.5 Hz, 1H), 2.47 (s, 3H), 2.12-1.64 (m, 9H), 1.55-1.29 (m, 2H), 0.88 (t, J=7.3 Hz, 3H).

### Example 212

LC/MS: m/z=600.3 [M+H]⁺; rt: 2.29 min (LC/MS-method A). ¹H NMR (400 MHz, DMSO-d6) δ ppm: 10.86 (s, 1H), 9.30 (d, J=7.9 Hz, 1H), 8.32 (d, J=5.2 Hz, 1H), 8.01 (br s, 1H), 7.55 (d, J=2.4 Hz, 1H), 7.05 (dd, J=5.2, 1.6 Hz, 1H), 4.77-4.67 (m, 2H), 4.48-4.37 (m, 1H), 3.72-3.63 (m, 1H, partially overlapping water), 3.06 (dd, J=10.0, 4.5 Hz, 1H), 2.27 (tt, J=8.3, 5.0 Hz, 1H), 2.13-1.64 (m, 9H), 1.56-1.30 (m, 2H), 1.20-1.06 (m, 2H), 1.04-0.92 (m, 2H), 0.88 (t, J=7.2 Hz, 3H).

### Example 213

LC/MS: m/z=586.3 [M+H]⁺; rt: 1.98 min (LC/MS-method A). ¹H NMR (400 MHz, DMSO-d6) δ ppm: 10.87 (br s, 1H), 8.60 (d, J=7.9 Hz, 1H), 8.32 (d, J=5.3 Hz, 1H), 7.99 (br s, 1H), 7.55 (d, J=2.4 Hz, 1H), 7.49 (d, J=2.0 Hz, 1H), 7.06 (dd, J=5.3, 1.6 Hz, 1H), 7.01 (d, J=2.0 Hz, 1H), 4.71 (dd, J=9.8, 5.9 Hz, 1H), 4.66-4.59 (m, 1H), 4.48-4.37 (m, 3H), 3.72-3.62 (m, 1H, overlapping water), 3.06 (dd, J=10.0, 4.5 Hz, 1H), 2.12-1.64 (m, 9H), 1.55-1.29 (m, 2H), 1.27 (t, J=7.1 Hz, 3H), 0.88 (t, J=7.2 Hz, 3H).

### Example 214

LC/MS: m/z=572.2 [M+H]⁺; rt: 1.87 min (LC/MS-method A). ¹H NMR (400 MHz, DMSO-d6) δ ppm: 10.90 (s, 1H), 8.60 (d, J=7.9 Hz, 1H), 8.32 (d, J=5.3 Hz, 1H), 7.99 (br s, 1H), 7.55 (d, J=2.4 Hz, 1H), 7.47 (d, J=2.0 Hz, 1H), 7.08-7.04 (m, 2H), 4.71 (dd, J=9.9, 5.9 Hz, 1H), 4.66-4.60 (m, 1H), 4.48-4.38 (m, 1H), 4.02 (s, 3H), 3.70-3.63 (m, 1H, partially overlapping water), 3.06 (dd, J=9.9, 4.5 Hz, 1H), 2.12-1.63 (m, 9H), 1.55-1.28 (m, 2H), 0.88 (t, J=7.2 Hz, 3H).

### Example 215

LC/MS: m/z=600.3 [M+H]⁺; rt: 2.10 min (LC/MS-method A). ¹H NMR (400 MHz, DMSO-d6) δ ppm: 10.81 (s, 1H), 8.57 (d, J=7.9 Hz, 1H), 8.31 (d, J=5.2 Hz, 1H), 8.00 (br s, 1H), 7.55 (d, J=2.4 Hz, 1H), 7.50 (d, J=2.0 Hz, 1H), 7.05 (dd, J=5.2, 1.6 Hz, 1H), 6.94 (d, J=2.0 Hz, 1H), 5.36 (hept, J=6.7 Hz, 1H), 4.71 (dd, J=9.8, 5.9 Hz, 1H), 4.64-4.58 (m, 1H), 4.48-4.37 (m, 1H), 3.71-3.63 (m, 1H, partially overlapping water), 3.06 (dd, J=10.0, 4.5 Hz, 1H), 2.12-1.62 (m, 9H), 1.55-1.28 (m, 8H), 0.88 (t, J=7.2 Hz, 3H).

### Example 216 N-((1S)-1-(4,4-difluorocyclohexyl)-2-oxo-2-((4-((2-oxo-5-(trifluoromethyl)-2,5-dihydro-1H-pyrrol-3-yl)methyl)pyridin-2-yl)amino)ethyl)-3-ethylisoxazole-4-carboxamide

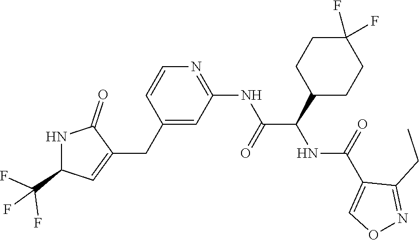 [SMILES: CCc1nocc1C(=O)N[C@@H](C(=O)Nc1cc(CC2=C[C@@H](C(F)(F)F)NC2=O)ccn1)C1CCC(F)(F)CC1]

Following the procedure described for Example 39, using 26 mg (2S)-2-amino-2-(4,4-difluorocyclohexyl)-N-(4-((2-oxo-5-(trifluoromethyl)-2,5-dihydro-1H-pyrrol-3-yl)methyl)-pyridin-2-yl)acetamide (Intermediate 79), 10 mg of the title compound was obtained. LC/MS: m/z=554.1 [M−H]⁻; rt: 1.94 min (LC/MS-method A). ¹H NMR (400 MHz, DMSO-de) δ ppm: 10.69 (s, 1H), 9.39 (s, 1H), 9.13 (br s, 1H), 8.52 (d, J=8.0 Hz, 1H), 8.24 (d, J=5.1 Hz, 1H), 7.97 (br s, 1H), 6.99 (dd, J=5.1, 1.5 Hz, 1H), 6.94-6.91 (m, 1H), 5.12-5.04 (m, 1H), 4.69-4.63 (m, 1H), 3.58 (s, 2H), 2.82 (q, J=7.5 Hz, 2H), 2.12-1.62 (m, 7H), 1.54-1.29 (m, 2H), 1.16 (t, J=7.5 Hz, 3H).

### Example 217: N—((S)-1-(4,4-difluorocyclohexyl)-2-oxo-2-((4-(((3S,5S)-2-oxo-5-(trifluoro-methyl)pyrrolidin-3-yl)methyl)pyridin-2-yl)amino)ethyl)-4-ethyloxazole-5-carboxamide

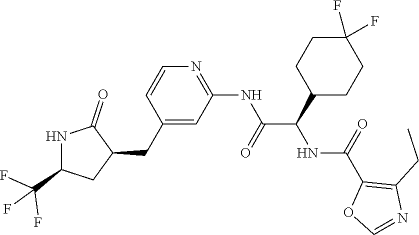 [SMILES: CCc1ncoc1C(=O)N[C@@H](C(=O)Nc1cc(C[C@H]2C[C@@H](C(F)(F)F)NC2=O)ccn1)C1CCC(F)(F)CC1]

Following the synthesis route described for Example 155, 33 mg of the title compound was obtained. LC/MS: m/z=558.2 [M+H]⁺; rt: 2.01 min (LC/MS-method A). ¹H NMR (400 MHz, DMSO-d₆) δ ppm: 10.92 (br s, 1H), 8.64 (s, 1H), 8.47 (s, 1H), 8.42 (d, J=8.1 Hz, 1H), 8.25 (d, J=5.2 Hz, 1H), 7.91 (br s, 1H), 7.11 (dd, J=5.2, 1.5 Hz, 1H), 4.64-4.57 (m, 1H), 4.31-4.20 (m, 1H), 3.05 (dd, J=14.0, 4.5 Hz, 1H), 2.84-2.74 (m, 3H), 2.64 (dd, J=14.0, 9.9 Hz, 1H), 2.40-2.30 (m, 1H), 2.05 (s, 3H), 1.93-1.65 (m, 4H), 1.60 (ddd, J=13.3, 8.6, 6.8 Hz, 1H), 1.52-1.39 (m, 1H), 1.37-1.24 (m, 1H), 1.13 (t, J=7.5 Hz, 3H).

Examples 218-222 were prepared analogously to Example 217. Examples 223-225 were prepared analogously to Example 217 using the procedure described for Example 33 in the final step.

| Ex. | Acid | Structure | Name | Yield |
|---|---|---|---|---|
| 218 | | | N-((S)-1-(4,4- | 22 mg, |
| | | | difluorocyclohexyl)-2-oxo-2- | 55% |
| | | | ((4-(((3S,5S)-2-oxo-5- | |
| | | | (trifluoromethyl)pyrrolidin-3- | |
| | | | yl)methyl)pyridin-2- | |
| | | | yl)amino)ethyl)-5- | |
| | | | ethylisoxazole-4-carboxamide | |
| 219 | | | N-((S)-1-(4,4- | 23 mg, |
| | | | difluorocyclohexyl)-2-oxo-2- | 56% |
| | | | ((4-(((3S,5S)-2-oxo-5- | |
| | | | (trifluoromethyl)pyrrolidin-3- | |
| | | | yl)methyl)pyridin-2- | |
| | | | yl)amino)ethyl)-5- | |
| | | | isopropylisoxazole-4- | |
| | | | carboxamide | |
| 220 | | | N-((S)-1-(4,4- | 32 mg, |
| | | | difluorocyclohexyl)-2-oxo-2- | 83% |
| | | | ((4-(((3S,5S)-2-oxo-5- | |
| | | | (trifluoromethyl)pyrrolidin-3- | |
| | | | yl)methyl)pyridin-2- | |
| | | | yl)amino)ethyl)-4-ethyl-1,2,5- | |
| | | | oxadiazole-3-carboxamide | |
| 221 | | | N-((S)-1-(4,4- | 35 mg, |
| | | | difluorocyclohexyl)-2-oxo-2- | 93% |
| | | | ((4-(((3S,5S)-2-oxo-5- | |
| | | | (trifluoromethyl)pyrrolidin-3- | |
| | | | yl)methyl)pyridin-2- | |
| | | | yl)amino)ethyl)-4-methyl-1,2,5- | |
| | | | oxadiazole-3-carboxamide | |
| 222 | | | 4-cyclopropyl-N-((S)-1-(4,4- | 31 mg, |
| | | | difluorocyclohexyl)-2-oxo-2- | 78% |
| | | | ((4-(((3S,5S)-2-oxo-5- | |
| | | | (trifluoromethyl)pyrrolidin-3- | |
| | | | yl)methyl)pyridin-2- | |
| | | | yl)amino)ethyl)-1,2,5- | |
| | | | oxadiazole-3-carboxamide | |
| 223 | | | N-((S)-1-(4,4- | 30 mg, |
| | | | difluorocyclohexyl)-2-oxo-2- | 78% |
| | | | ((4-(((3S,5S)-2-oxo-5- | |
| | | | (trifluoromethyl)pyrrolidin-3- | |
| | | | yl)methyl)pyridin-2- | |
| | | | yl)amino)ethyl)-1-ethyl-1H- | |
| | | | pyrazole-5-carboxamide | |
| 224 | | | N-((S)-1-(4,4- | 28 mg, |
| | | | difluorocyclohexyl)-2-oxo-2- | 74% |
| | | | ((4-(((3S,5S)-2-oxo-5- | |
| | | | (trifluoromethyl)pyrrolidin-3- | |
| | | | yl)methyl)pyridin-2- | |
| | | | yl)amino)ethyl)-1-methyl-1H- | |
| | | | pyrazole-5-carboxamide | |
| 225 | | | N-((S)-1-(4,4- | 35 mg, |
| | | | difluorocyclohexyl)-2-oxo-2- | 88% |
| | | | ((4-(((3S,5S)-2-oxo-5- | |
| | | | (trifluoromethyl)pyrrolidin-3- | |
| | | | yl)methyl)pyridin-2- | |
| | | | yl)amino)ethyl)-1-isopropyl- | |
| | | | 1H-pyrazole-5-carboxamide | |

### Example 218

LC/MS: m/z=558.2 [M+H]⁺; rt: 2.11 min (LC/MS-method A). ¹H NMR (400 MHz, DMSO-d6) δ ppm: 10.80 (br s, 1H), 9.08 (s, 1H), 8.63 (s, 1H), 8.47 (d, J=7.9 Hz, 1H), 8.24 (d, J=5.2 Hz, 1H), 7.94 (br s, 1H), 7.07 (dd, J=5.2, 1.5 Hz, 1H), 4.69-4.61 (m, 1H), 4.30-4.20 (m, 1H), 3.10-3.01 (m, 3H), 2.83-2.72 (m, 1H), 2.62 (dd, J=14.0, 9.9 Hz, 1H), 2.39-2.29 (m, 1H), 2.13-1.64 (m, 7H), 1.60 (ddd, J=13.3, 8.6, 6.7 Hz, 1H), 1.54-1.29 (m, 2H), 1.19 (t, J=7.6 Hz, 3H).

### Example 219

LC/MS: m/z=572.2 [M+H]⁺; rt: 2.24 min (LC/MS-method A). ¹H NMR (400 MHz, DMSO-d6) δ ppm: 10.83 (br s, 1H), 9.07 (s, 1H), 8.63 (s, 1H), 8.48 (d, J=7.9 Hz, 1H), 8.25 (d, J=5.2 Hz, 1H), 7.93 (br s, 1H), 7.08 (dd, J=5.2, 1.5 Hz, 1H), 4.68-4.61 (m, 1H), 4.30-4.20 (m, 1H), 3.89-3.74 (m, 1H, overlapping water), 3.05 (dd, J=14.0, 4.4 Hz, 1H), 2.83-2.72 (m, 1H), 2.63 (dd, J=14.0, 9.8 Hz, 1H), 2.40-2.29 (m, 1H), 2.13-1.64 (m, 7H), 1.60 (ddd, J=13.3, 8.5, 6.8 Hz, 1H), 1.55-1.30 (m, 2H), 1.26-1.21 (m, 6H).

### Example 220

LC/MS: m/z=559.2 [M+H]⁺; rt: 2.14 min (LC/MS-method A). ¹H NMR (400 MHz, DMSO-d6) δ ppm: 10.88 (br s, 1H), 9.28 (d, J=8.0 Hz, 1H), 8.63 (s, 1H), 8.25 (d, J=5.2 Hz, 1H), 7.94 (br s, 1H), 7.08 (br d, J=5.2 Hz, 1H), 4.75-4.68 (m, 1H), 4.31-4.20 (m, 1H), 3.05 (dd, J=14.0, 4.4 Hz, 1H), 2.90 (q, J=7.5 Hz, 2H), 2.84-2.73 (m, 1H), 2.63 (dd, J=14.0, 9.8 Hz, 1H), 2.40-2.29 (m, 1H), 2.13-1.97 (m, 3H), 1.93-1.65 (m, 4H), 1.60 (ddd, J=13.2, 8.6, 6.8 Hz, 1H), 1.55-1.30 (m, 2H), 1.24 (t, J=7.5 Hz, 3H).

### Example 221

LC/MS: m/z=545.2 [M+H]⁺; rt: 1.99 min (LC/MS-method A). ¹H NMR (400 MHz, DMSO-d6) δ ppm: 10.86 (br s, 1H), 9.25 (d, J=8.0 Hz, 1H), 8.63 (s, 1H), 8.25 (d, J=5.2 Hz, 1H), 7.95 (br s, 1H), 7.08 (dd, J=5.2, 1.5 Hz, 1H), 4.75-4.67 (m, 1H), 4.31-4.20 (m, 1H), 3.05 (dd, J=14.0, 4.5 Hz, 1H), 2.79 (qd, J=9.4, 4.4 Hz, 1H), 2.63 (dd, J=14.0, 9.7 Hz, 1H), 2.47 (s, 3H), 2.40-2.29 (m, 1H), 2.13-1.97 (m, 3H), 1.93-1.65 (m, 4H), 1.60 (ddd, J=13.2, 8.6, 6.8 Hz, 1H), 1.55-1.29 (m, 2H).

### Example 222

LC/MS: m/z=571.1 [M+H]⁺; rt: 2.18 min (LC/MS-method A). ¹H NMR (400 MHz, DMSO-d6) δ ppm: 10.94 (br s, 1H), 9.34 (d, J=8.0 Hz, 1H), 8.64 (s, 1H), 8.26 (d, J=5.2 Hz, 1H), 7.94 (br s, 1H), 7.10 (dd, J=5.2, 1.5 Hz, 1H), 4.77-4.70 (m, 1H), 4.31-4.20 (m, 1H), 3.06 (dd, J=14.0, 4.5 Hz, 1H), 2.85-2.74 (m, 1H), 2.64 (dd, J=14.0, 9.7 Hz, 1H), 2.41-2.23 (m, 2H), 2.13-1.98 (m, 3H), 1.93-1.66 (m, 4H), 1.60 (ddd, J=13.2, 8.6, 6.7 Hz, 1H), 1.55-1.31 (m, 2H), 1.18-1.08 (m, 2H), 1.02-0.94 (m, 2H).

### Example 223

LC/MS: m/z=557.2 [M+H]⁺; rt: 1.86 min (LC/MS-method A). ¹H NMR (400 MHz, DMSO-d6) δ ppm: 11.08 (br s, 1H), 8.68-8.62 (m, 2H), 8.27 (d, J=5.4 Hz, 1H), 7.90 (br s, 1H), 7.49 (d, J=2.0 Hz, 1H), 7.15 (br d, J=5.4 Hz, 1H), 7.02 (d, J=2.0 Hz, 1H), 4.65-4.58 (m, 1H), 4.45 (q, J=7.1 Hz, 2H), 4.31-4.20 (m, 1H), 3.07 (dd, J=14.0, 4.5 Hz, 1H), 2.85-2.75 (m, 1H), 2.67 (dd, J=14.0, 9.7 Hz, 1H), 2.41-2.31 (m, 1H), 2.13-1.98 (m, 3H), 1.96-1.66 (m, 4H), 1.60 (ddd, J=13.2, 8.6, 6.8 Hz, 1H), 1.55-1.30 (m, 2H), 1.27 (t, J=7.1 Hz, 3H).

### Example 224

LC/MS: m/z=543.2 [M+H]⁺; rt: 1.75 min (LC/MS-method A). ¹H NMR (400 MHz, DMSO-d6) δ ppm: 11.06 (br s, 1H), 8.67-8.63 (m, 2H), 8.26 (d, J=5.3 Hz, 1H), 7.91 (br s, 1H), 7.48 (d, J=2.1 Hz, 1H), 7.14 (br d, J=5.3 Hz, 1H), 7.06 (d, J=2.1 Hz, 1H), 4.65-4.58 (m, 1H), 4.31-4.20 (m, 1H), 4.02 (s, 3H), 3.07 (dd, J=14.0, 4.5 Hz, 1H), 2.85-2.75 (m, 1H), 2.66 (dd, J=14.0, 9.8 Hz, 1H), 2.41-2.30 (m, 1H), 2.13-1.97 (m, 3H), 1.96-1.65 (m, 4H), 1.60 (ddd, J=13.2, 8.6, 6.8 Hz, 1H), 1.55-1.29 (m, 2H).

### Example 225

LC/MS: m/z=571.2 [M+H]⁺; rt: 1.98 min (LC/MS-method A). ¹H NMR (400 MHz, DMSO-d6) δ ppm: 11.05 (br s, 1H), 8.67-8.61 (m, 2H), 8.26 (d, J=5.3 Hz, 1H), 7.90 (br s, 1H), 7.51 (d, J=2.0 Hz, 1H), 7.14 (br d, J=5.3 Hz, 1H), 6.96 (d, J=2.0 Hz, 1H), 5.36 (hept, J=6.6 Hz, 1H), 4.64-4.57 (m, 1H), 4.31-4.20 (m, 1H), 3.07 (dd, J=14.0, 4.5 Hz, 1H), 2.85-2.75 (m, 1H), 2.66 (dd, J=14.0, 9.7 Hz, 1H), 2.41-2.31 (m, 1H), 2.13-1.98 (m, 3H), 1.96-1.66 (m, 4H), 1.61 (ddd, J=13.2, 8.6, 6.8 Hz, 1H), 1.55-1.30 (m, 8H).

### Example 226: N—((S)-1,1-Dicyclopropyl-3-((4-((S)-2-methoxy-1-((S)-2-oxo-4-(trifluoro-methyl)imidazolidin-1-yl)ethyl)pyridin-2-yl)amino)-3-oxopropan-2-yl)-4-methyl-1,2,5-oxadiazole-3-carboxamide

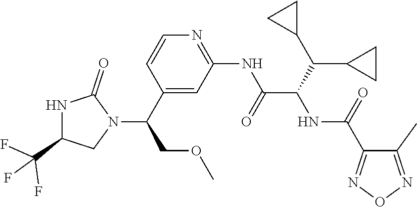 [SMILES: COC[C@H](c1ccnc(NC(=O)[C@@H](NC(=O)c2nonc2C)C(C2CC2)C2CC2)c1)N1C[C@@H](C(F)(F)F)NC1=O]

To a mixture of (S)-2-amino-3,3-dicyclopropyl-N-(4-((S)-2-methoxy-1-((S)-2-oxo-4-(trifluoromethyl)imidazolidin-1-yl)ethyl)pyridin-2-yl)propanamide hydrochloride (Intermediate 54; 15 mg), 1-methyl-1H-pyrazole-5-carboxylic acid (4 mg) and DCM (1 ml) DIPEA (26 μl) and T3P (53 μl) were added. After stirring for 30 min the mixture was concentrated in vacuo and the residue was purified by preparative RP HPLC (Agilent Prep C18, 21.2 mm×250 mm, 10 μm; 50 ml/min, from 90% H₂O/10% ACN to 10% H₂O/90% ACN in 12.5 min). The pure product containing fractions were combined, the ACN was partly removed and the residue freeze dried from ACN/H₂O to yield 11 mg of the title compound. LC/MS: m/z=566.2 [M+H]⁺; rt: 2.14 min (LC/MS-method A). ¹H NMR (400 MHz, DMSO-de) 6 ppm: 10.74 (s, 1H), 9.04 (d, J=8.8 Hz, 1H), 8.29 (d, J=5.1 Hz, 1H), 8.01 (br s, 1H), 7.63 (d, J=2.4 Hz, 1H), 7.00 (dd, J=5.1, 1.5 Hz, 1H), 5.03 (dd, J=8.0, 5.2 Hz, 1H), 5.00-4.94 (m, 1H), 4.49-4.38 (m, 1H), 3.82 (dd, J=10.5, 8.0 Hz, 1H), 3.79-3.71 (m, 2H), 3.31 (s, 3H, overlapping water), 3.27 (dd, J=10.0, 3.9 Hz, 1H, partially overlapping water), 2.48 (s, 3H), 0.96-0.72 (m, 3H), 0.52-0.15 (m, 8H).

Using (S)-2-amino-3,3-dicyclopropyl-N-(4-((S)-2-methoxy-1-((S)-2-oxo-4-(trifluoromethyl)imidazolidin-1-yl)ethyl)pyridin-2-yl)propanamide hydrochloride (Intermediate 54) as starting material, analogously to Example 226 were prepared:

| Ex. | Acid | Structure | Name | Yield |
|---|---|---|---|---|
| 227 | | | 4-cyclopropyl-N-((S)-1,1- | 11 mg, |
| | | | dicyclopropyl-3-((4-((S)-2- | 64% |
| | | | methoxy-1-((S)-2-oxo-4- | |
| | | | (trifluoromethyl)imidazolidin- | |
| | | | 1-yl)ethyl)pyridin-2- | |
| | | | yl)amino)-3-oxopropan-2- | |
| | | | yl)-1,2,5-oxadiazole-3- | |
| | | | carboxamide | |
| 228 | | | N-((S)-1,1-dicyclopropyl-3- | 11 mg, |
| | | | ((4-((S)-2-methoxy-1-((S)- | 69% |
| | | | 2-oxo-4- | |
| | | | (trifluoromethyl)imidazolidin- | |
| | | | 1-yl)ethyl)pyridin-2- | |
| | | | yl)amino)-3-oxopropan-2- | |
| | | | yl)-1-methyl-1H-pyrazole- | |
| | | | 5-carboxamide | |
| 229 | | | N-((S)-1,1-dicyclopropyl-3- | 11 mg, |
| | | | ((4-((S)-2-methoxy-1-((S)- | 61% |
| | | | 2-oxo-4-(trifluoromethyl) | |
| | | | imidazolidin-1-yl)ethyl) | |
| | | | pyridin-2-yl)amino)-3- | |
| | | | oxopropan-2-yl)-1- | |
| | | | isopropyl-1H-pyrazole-5- | |
| | | | carboxamide | |

### Example 227

LC/MS: m/z=592.2 [M+H]⁺; rt: 2.33 min (LC/MS-method A). ¹H NMR (400 MHz, DMSO-d₆) δ ppm: 10.74 (s, 1H), 9.14 (d, J=8.8 Hz, 1H), 8.29 (d, J=5.3 Hz, 1H), 8.02 (br s, 1H), 7.63 (d, J=2.3 Hz, 1H), 7.00 (dd, J=5.3, 11.6 Hz, 1H), 5.06-4.96 (m, 2H), 4.49-4.38 (m, 1H), 3.83 (dd, J=10.5, 8.0 Hz, 1H), 3.79-3.71 (m, 2H), 3.38-3.23 (m, 4H, overlapping water), 2.28 (tt, J=8.4, 5.0 Hz, 1H), 1.17-1.08 (m, 2H), 1.03-0.71 (m, 5H), 0.52-0.13 (m, 8H).

### Example 228

LC/MS: m/z=564.2 [M+H]⁺; rt: 1.87 min (LC/MS-method A). ¹H NMR (400 MHz, DMSO-d6) δ ppm: 10.65 (s, 1H), 8.39 (d, J=8.7 Hz, 1H), 8.29 (d, J=5.3 Hz, 1H), 8.03 (br s, 1H), 7.62 (d, J=2.3 Hz, 1H), 7.47 (d, J=2.1 Hz, 1H), 7.03 (d, J=2.1 Hz, 1H), 6.98 (dd, J=5.3, 1.5 Hz, 1H), 5.02 (dd, J=8.0, 5.3 Hz, 1H), 4.95-4.88 (m, 1H), 4.48-4.38 (m, 1H), 4.03 (s, 3H), 3.82 (dd, J=10.5, 8.0 Hz, 1H), 3.78-3.70 (m, 2H), 3.30 (s, 3H, overlapping water), 3.26 (dd, J=9.9, 3.9 Hz, 1H, partially overlapping water), 0.99-0.70 (m, 3H), 0.50-0.10 (m, 8H).

### Example 229

LC/MS: m/z=592.2 [M+H]⁺; rt: 2.10 min (LC/MS-method A). ¹H NMR (400 MHz, DMSO-d₆) δ ppm: 10.63 (s, 1H), 8.39 (d, J=8.7 Hz, 1H), 8.29 (d, J=5.2 Hz, 1H), 8.02 (br s, 1H), 7.62 (d, J=2.3 Hz, 1H), 7.50 (d, J=2.0 Hz, 1H), 6.99 (dd, J=5.2, 1.5 Hz, 1H), 6.91 (d, J=2.0 Hz, 1H), 5.38 (hept, J=6.6 Hz, 1H), 5.02 (dd, J=8.9, 5.4 Hz, 1H), 4.93-4.86 (m, 1H), 4.48-4.38 (m, 1H), 3.82 (dd, J=10.5, 8.0 Hz, 1H), 3.78-3.70 (m, 2H), 3.31 (s, 3H, overlapping water), 3.26 (dd, J=9.9, 3.9 Hz, 1H, partially overlapping water), 1.37 (d, J=6.6 Hz, 3H), 1.34 (d, J=6.6 Hz, 3H), 1.00-0.69 (m, 3H), 0.50-0.10 (m, 8H).

### Example 230: N—((S)-1,1-dicyclopropyl-3-oxo-3-((4-(((3S,5S)-2-oxo-5-(trifluoromethyl)-pyrrolidin-3-yl)methyl)pyridin-2-yl)amino)propan-2-yl)-1-ethyl-1H-pyrazole-5-carboxamide

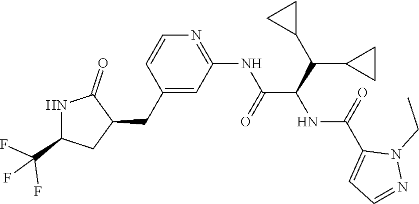 [SMILES: CCn1nccc1C(=O)N[C@@H](C(=O)Nc1cc(C[C@H]2C[C@@H](C(F)(F)F)NC2=O)ccn1)C(C1CC1)C1CC1]

Following the synthesis route described for Example 155, 3.3 mg of the title compound was obtained. LC/MS: m/z=533.2 [M+H]⁺; rt: 1.95 min (LC/MS-method A). ¹H NMR (400 MHz, DMSO-de) δ ppm: 10.74 (br s, 1H), 8.62 (s, 1H), 8.42 (d, J=8.7 Hz, 1H), 8.23 (d, J=5.2 Hz, 1H), 7.93 (br s, 1H), 7.49 (d, J=2.0 Hz, 1H), 7.05 (dd, J=5.2, 1.5 Hz, 1H), 6.99 (d, J=2.0 Hz, 1H), 4.94-4.87 (m, 1H), 4.52-4.41 (m, 2H), 4.30-4.19 (m, 1H), 3.05 (dd, J=13.7, 4.3 Hz, 1H), 2.81-2.70 (m, 1H), 2.60 (dd, J=13.7, 10.2 Hz, 1H), 2.36-2.26 (m, 1H), 1.59 (ddd, J=13.3, 8.3, 6.6 Hz, 1H), 1.28 (t, J=7.1 Hz, 3H), 1.01-0.90 (m, 1H), 0.90-0.70 (m, 2H), 0.50-0.10 (m, 8H).

### Example 231: 4-Cyclopropyl-N-((1S)-1-((1r,4S)-4-methylcyclohexyl)-2-oxo-2-((4-(((S)-2-oxo-4-(trifluoromethyl)imidazolidin-1-yl)methyl-d)pyridin-2-yl)amino)ethyl)-1,2,5-oxadiazole-3-carboxamide

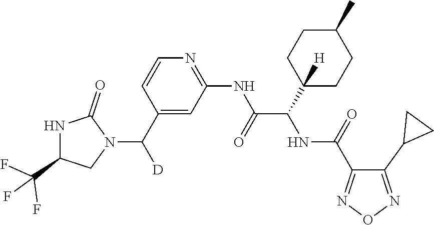

To a mixture of (2S)-2-amino-2-((1 r,4S)-4-methylcyclohexyl)-N-(4-(((S)-2-oxo-4-(trifluoromethyl)imidazolidin-1-yl)methyl-d)pyridin-2-yl)acetamide hydrochloride (Intermediate 55, 20 mg), 4-cyclopropyl-1,2,5-oxadiazole-3-carboxylic acid (8 mg) and DCM (3 ml), DIPEA (28 μl) and T3P (85 μl) were added. After 1.5 h the mixture was concentrated in vacuo and the residue was purified by preparative RP HPLC (Purosphere® STAR-RP18, 25 mm×250 mm, 10 μm; 30 ml/min, from 95% H₂O/5% ACN to 10% H₂O/90% ACN in 45 min). The pure product containing fractions were combined, the ACN was partly removed and the residue freeze dried to yield 13 mg of the title compound. LC/MS: m/z=551.3 [M+H]⁺; rt: 2.43 min (LC/MS-method A). ¹H NMR (400 MHz, DMSO-d₆) δ ppm: 10.72 (s, 1H), 9.16 (d, J=8.1 Hz, 1H), 8.30 (d, J=5.1 Hz, 1H), 7.98 (br s, 1H), 7.65 (d, J=2.4 Hz, 1H), 6.96 (dd, J=5.1, 1.5 Hz, 1H), 4.66-4.58 (m, 1H), 4.47-4.19 (m, 2H), 3.63-3.54 (m, 1H), 3.39-3.27 (m, 1H, overlapping water), 2.27 (tt, J=8.4, 5.0 Hz, 1H), 1.86-1.74 (m, 2H), 1.73-1.55 (m, 3H), 1.35-1.18 (m, 2H), 1.17-1.03 (m, 3H), 1.02-0.94 (m, 2H), 0.93-0.80 (m, 5H).

### Example 232: 1-Methyl-N-((1S)-1-((1r,4S)-4-methylcyclohexyl)-2-oxo-2-((4-(((S)-2-oxo-4-(trifluoromethyl)imidazolidin-1-VI)methyl-d)pyridin-2-yl)amino)ethyl)-1H-pyrazole-5-carboxamide

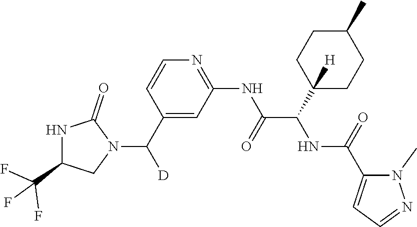

Following the procedure described in Example 231, and using 1-methyl-1H-pyrazole-5-carboxylic acid, 11 mg (53%) of the title compound was obtained. LC/MS: m/z=523.3 [M+H]⁺; rt: 1.99 min (LC/MS-method A). ¹H NMR (400 MHz, DMSO-de) δ ppm: 10.61 (s, 1H), 8.43 (d, J=8.0 Hz, 1H), 8.28 (d, J=5.2 Hz, 1H), 7.98 (br s, 1H), 7.64 (d, J=2.4 Hz, 1H), 7.46 (d, J=2.1 Hz, 1H), 7.03 (d, J=2.1 Hz, 1H), 6.95 (dd, J=5.2, 1.5 Hz, 1H), 4.55-4.17 (m, 3H), 4.01 (s, 3H), 3.62-3.54 (m, 1H), 3.38-3.26 (m, 1H, overlapping water), 1.87-1.63 (m, 4H), 1.57 (d, J=12.9 Hz, 1H), 1.36-1.16 (m, 2H), 1.13-0.99 (m, 1H), 0.93-0.79 (m, 5H).

### Example 233: 4-Cyclopropyl-N-((1S)-2-((4-((R or S)-2-hydroxy-1-((S)-2-oxo-4-(trifluoro-methyl)imidazolidin-1-yl)ethyl)pyridin-2-yl)amino)-1-((1r,4S)-4-methylcyclohexyl)-2-oxoethyl)-1,2,5-oxadiazole-3-carboxamide (DS1)

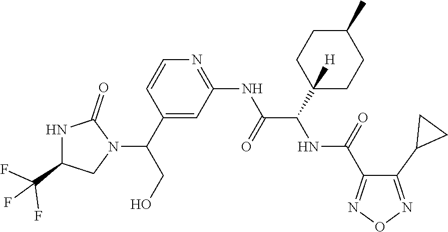

To a mixture of (2S)-2-amino-N-(4-((R or S)-2-hydroxy-1-((S)-2-oxo-4-(trifluoromethyl)imidazolidin-1-yl)ethyl)pyridin-2-yl)-2-((1 r,4S)-4-methylcyclohexyl)-acetamide hydrochloride (Intermediate 56-DS1, 10 mg), 4-cyclopropyl-1,2,5-oxadiazole-3-carboxylic acid (4 mg) and DCM (1 ml), DIPEA (20 μl) and T3P (40 μl) were added. After 20 min the mixture was concentrated in vacuo and the residue was purified by preparative RP HPLC (Agilent Prep C18, 21.2 mm×250 mm, 10 μm; 50 ml/min, from 90% H₂O/10% ACN to 10% H₂O/90% ACN in 12.5 min). The pure product containing fractions were combined, the ACN was partly removed and the residue freeze dried to yield 3 mg of the title compound. LC/MS: m/z=580.1 [M+H]⁺; rt: 2.26 min (LC/MS-method A). ¹H NMR (400 MHz, DMSO-d₆) δ ppm: 10.69 (s, 1H), 9.15 (d, J=8.0 Hz, 1H), 8.29 (d, J=5.2 Hz, 1H), 8.00 (br s, 1H), 7.56 (d, J=2.4 Hz, 1H), 7.01 (dd, J=5.2, 1.6 Hz, 1H), 5.12-5.06 (m, 1H), 4.82 (dd, J=8.1, 5.4 Hz, 1H), 4.65-4.58 (m, 1H), 4.47-4.37 (m, 1H), 3.90-3.75 (m, 3H), 3.35-3.28 (m, 1H, overlapping water), 2.27 (tt, J=8.4, 5.0 Hz, 1H), 1.86-1.74 (m, 2H), 1.73-1.56 (m, 3H), 1.33-1.17 (m, 2H), 1.16-1.02 (m, 3H), 1.01-0.95 (m, 2H), 0.93-0.80 (m, 5H).

### Example 234: 4-Cyclopropyl-N-((1S)-2-((4-((R or S)-2-hydroxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)-1,2,5-oxadiazole-3-carboxamide (DS2)

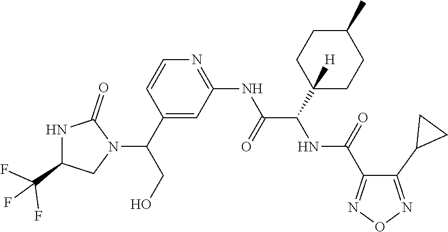

Following the procedure described in Example 233, and using (2S)-2-amino-N-(4-((R or S)-2-hydroxy-1-((S)-2-oxo-4-(trifluoromethyl)imidazolidin-1-yl)ethyl)pyridin-2-yl)-2-((1r,4S)-4-methylcyclohexyl)acetamide hydrochloride salt (Intermediate 56-DS2, 6 mg), 4 mg (53%) of the title compound was obtained. LC/MS: m/z=580.2 [M+H]⁺; rt: 2.29 min (LC/MS-method A). ¹H NMR (400 MHz, DMSO-de) δ ppm: 10.72 (s, 1H), 9.16 (d, J=8.1 Hz, 1H), 8.29 (d, J=5.2 Hz, 1H), 8.05 (br s, 1H), 7.53 (d, J=2.4 Hz, 1H), 7.08 (dd, J=5.2, 1.6 Hz, 1H), 5.07-5.03 (m, 1H), 4.82-4.77 (m, 1H), 4.66-4.59 (m, 1H), 4.45-4.35 (m, 1H), 3.89-3.74 (m, 2H), 3.62-3.54 (m, 2H), 2.28 (tt, J=8.4, 5.0 Hz, 1H), 1.86-1.75 (m, 2H), 1.74-1.56 (m, 3H), 1.34-1.18 (m, 2H), 1.17-1.03 (m, 3H), 1.02-0.94 (m, 2H), 0.93-0.80 (m, 5H).

### Example 235: 4-Cyclopropyl-N-((1S)-2-((4-(cyclopropyl(4,4,4-trifluorobutanamido)-methyl)pyridin-2-yl)amino)-1-((1r,4S)-4-methylcyclohexyl)-2-oxoethyl)-1,2,5-oxadiazole-3-carboxamide

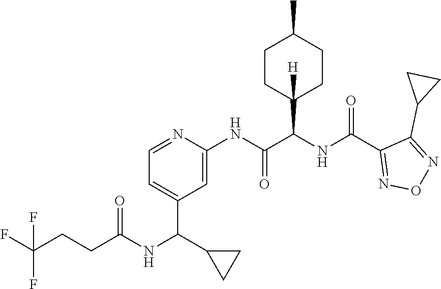

Was prepared by the synthesis route described for Example 155 and 23 mg of the title compound was obtained. LC/MS: m/z=577.3 [M+H]⁺; rt: 2.69 min (LC/MS-method A). ¹H NMR (400 MHz, DMSO-de) δ ppm: 10.76 (s, 1H), 9.17 (d, J=8.1 Hz, 1H), 8.74 (d, J=7.7 Hz, 1H), 8.26 (d, J=5.2 Hz, 1H), 8.08 (br s 1H), 7.12 (dd, J=5.2, 1.5 Hz, 1H), 4.66-4.58 (m, 1H), 4.19-4.12 (m, 1H), 2.55-2.36 (m, 4H, overlapping solvent), 2.28 (tt, J=8.4, 5.0 Hz, 1H), 1.87-1.75 (m, 2H), 1.74-1.56 (m, 3H), 1.35-1.18 (m, 2H), 1.17-1.03 (m, 4H), 1.02-0.94 (m, 2H), 0.93-0.80 (m, 5H), 0.58-0.47 (m, 2H), 0.45-0.30 (m, 2H).

### Example 236: N-((1S)-2-((4-(cyclopropyl(4,4,4-trifluorobutanamido)methyl)pyridin-2-yl)amino)-1-((1r,4S)-4-methylcyclohexyl)-2-oxoethyl)-1-methyl-1H-pyrazole-5-carboxamide

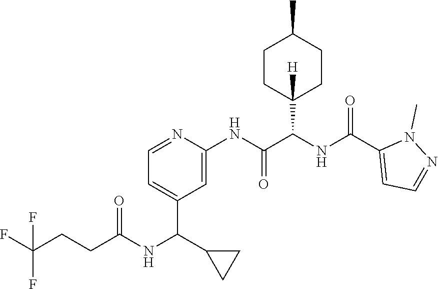

Was prepared by the synthesis route described for Example 155 and 35 mg of the title compound was obtained. LC/MS: m/z=549.3 [M+H]⁺; rt: 2.27 min (LC/MS-method A). ¹H NMR (400 MHz, DMSO-d₆) δ ppm: 10.85 (br s, 1H), 8.76 (d, J=7.7 Hz, 1H), 8.50 (d, J=8.0 Hz, 1H), 8.27 (d, J=5.3 Hz, 1H), 8.04 (br s, 1H), 7.46 (d, J=2.1 Hz, 1H), 7.16 (dd, J=5.3, 1.6 Hz, 1H), 7.05 (d, J=2.1 Hz, 1H), 4.53-4.46 (m, 1H), 4.20-4.12 (m, 1H), 4.01 (s, 3H), 2.55-2.36 (m, 4H, overlapping solvent), 1.89-1.54 (m, 5H), 1.36-1.17 (m, 2H), 1.15-1.00 (m, 2H), 0.95-0.79 (m, 5H), 0.59-0.46 (m, 2H), 0.46-0.30 (m, 2H).

### Example 237: 4-methyl-N—((S)-1-((1r,4S)-4-methylcyclohexyl)-2-oxo-2-((4-((R)-1-((S)-2-oxo-4-(trifluoromethyl)imidazolidin-1-yl)ethyl)pyridin-2-yl)amino)ethyl)-1,2,5-oxadiazole-3-carboxamide

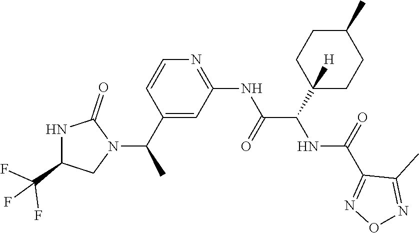

Following the procedure described for Ex. 39 using 41 mg (S)-2-amino-2-((1 r,4S)-4-methylcyclohexyl)-N-(4-((R)-1-((S)-2-oxo-4-(trifluoromethyl)imidazolidin-1-yl)ethyl)-pyridin-2-yl)acetamide (Intermediate 70), 38 mg of the title compound was obtained. LC/MS: m/z=538.2 [M+H]⁺; rt: 2.29 min (LC/MS-method A). ¹H NMR (400 MHz, DMSO-de) δ ppm: 10.81 (s, 1H), 9.10 (d, J=8.1 Hz, 1H), 8.31 (d, J=5.3 Hz, 1H), 7.99 (br s, 1H), 7.60 (d, J=2.4 Hz, 1H), 7.02 (dd, J=5.3, 1.6 Hz, 1H), 4.97 (q, J=7.1 Hz, 1H), 4.64-4.56 (m, 1H), 4.46-4.34 (m, 1H), 3.77-3.67 (m, 1H, overlapping water), 3.08 (dd, J=9.9, 4.1 Hz, 1H), 2.47 (s, 3H), 1.88-1.75 (m, 2H), 1.73-1.64 (m, 2H), 1.64-1.55 (m, 1H), 1.45 (d, J=7.2 Hz, 3H), 1.35-1.17 (m, 2H), 1.15-1.02 (m, 1H), 0.93-0.80 (in, 5H).

Example 238 was prepared analogously to Example 237. Examples 239-241 were prepared analogously to Example 237 using the procedure described for Example 33.

| Ex. | Acid | Structure | Name | Yield |
|---|---|---|---|---|
| 238 | | | 4-cyclopropyl-N-((S)-1- | 38 mg, |
| | | | ((1r,4S)-4- | 73% |
| | | | methylcyclohexyl)-2-oxo-2- | |
| | | | ((4-((R)-1-((S)-2-oxo-4- | |
| | | | (trifluoromethyl)imidazolidin- | |
| | | | 1-yl)ethyl)pyridin-2- | |
| | | | yl)amino)ethyl)-1,2,5- | |
| | | | oxadiazole-3-carboxamide | |
| 239 | | | 1-ethyl-N-((S)-1-((1r,4S)-4- | 45 mg, |
| | | | methylcyclohexyl)-2-oxo-2- | 85% |
| | | | ((4-((R)-1-((S)-2-oxo-4- | |
| | | | (trifluoromethyl)imidazolidin- | |
| | | | 1-yl)ethyl)pyridin-2- | |
| | | | yl)amino)ethyl)-1H- | |
| | | | pyrazole-5-carboxamide | |
| 240 | | | 1-methyl-N-((S)-1-((1r,4S)- | 38 mg, |
| | | | 4-methylcyclohexyl)-2-oxo- | 73% |
| | | | 2-((4-((R)-1-((S)-2-oxo-4- | |
| | | | (trifluoromethyl)imidazolidin- | |
| | | | 1-yl)ethyl)pyridin-2- | |
| | | | yl)amino)ethyl)-1H- | |
| | | | pyrazole-5-carboxamide | |
| 241 | | | 1-isopropyl-N-((S)-1- | 48 mg, |
| | | | ((1r,4S)-4- | 88% |
| | | | methylcyclohexyl)-2-oxo-2- | |
| | | | ((4-((R)-1-((S)-2-oxo-4- | |
| | | | (trifluoromethyl)imidazolidin- | |
| | | | 1-yl)ethyl)pyridin-2- | |
| | | | yl)amino)ethyl)-1H- | |
| | | | pyrazole-5-carboxamide | |

### Example 238

LC/MS: m/z=564.2 [M+H]⁺; rt: 2.46 min (LC/MS-method A). ¹H NMR (400 MHz, DMSO-d6) δ ppm: 10.81 (s, 1H), 9.17 (d, J=8.1 Hz, 1H), 8.31 (d, J=5.3 Hz, 1H), 7.99 (br s, 1H), 7.60 (d, J=2.5 Hz, 1H), 7.02 (dd, J=5.3, 1.6 Hz, 1H), 4.98 (q, J=7.1 Hz, 1H), 4.66-4.58 (m, 1H), 4.45-4.34 (m, 1H), 3.80-3.67 (m, 1H, overlapping water), 3.08 (dd, J=10.0, 4.1 Hz, 1H), 2.27 (tt, J=8.4, 5.0 Hz, 1H), 1.87-1.75 (m, 2H), 1.73-1.64 (m, 2H), 1.60 (d, J=12.9 Hz, 1H), 1.45 (d, J=7.1 Hz, 3H), 1.34-1.18 (m, 2H), 1.17-1.04 (m, 3H), 1.02-0.94 (m, 2H), 0.93-0.79 (m, 5H).

### Example 239

LC/MS: m/z=564.2 [M+H]⁺; rt: 2.15 min (LC/MS-method A). ¹H NMR (400 MHz, DMSO-d6) δ ppm: 10.73 (br s, 1H), 8.46 (d, J=8.0 Hz, 1H), 8.30 (d, J=5.3 Hz, 1H), 7.99 (br s, 1H), 7.59 (d, J=2.4 Hz, 1H), 7.48 (d, J=2.0 Hz, 1H), 7.02 (dd, J=5.3, 1.6 Hz, 1H), 7.00 (d, J=2.0 Hz, 1H), 4.97 (q, J=7.1 Hz, 1H), 4.54-4.34 (m, 4H), 3.75-3.60 (m, 1H, overlapping water), 3.07 (dd, J=10.0, 4.1 Hz, 1H), 1.87-1.74 (m, 2H), 1.73-1.63 (m, 2H), 1.62-1.54 (m, 1H), 1.44 (d, J=7.1 Hz, 3H), 1.35-1.16 (m, 5H), 1.14-1.00 (m, 1H), 0.94-0.75 (m, 5H).

### Example 240

LC/MS: m/z=536.2 [M+H]⁺; rt: 2.04 min (LC/MS-method A). ¹H NMR (400 MHz, DMSO-d6) δ ppm: 10.76 (br s, 1H), 8.46 (d, J=8.0 Hz, 1H), 8.30 (d, J=5.2 Hz, 1H), 7.98 (br s, 1H), 7.60 (d, J=2.4 Hz, 1H), 7.46 (d, J=2.1 Hz, 1H), 7.04 (d, J=2.1 Hz, 1H), 7.02 (dd, J=5.2, 1.6 Hz, 1H), 4.97 (q, J=7.1 Hz, 1H), 4.54-4.47 (m, 1H), 4.45-4.34 (m, 1H), 4.01 (s, 3H), 3.85-3.60 (m, 1H, overlapping water), 3.08 (dd, J=10.0, 4.1 Hz, 1H), 1.87-1.75 (m, 2H), 1.74-1.63 (m, 2H), 1.62-1.54 (m, 1H), 1.44 (d, J=7.1 Hz, 3H), 1.36-1.16 (m, 2H), 1.13-1.00 (m, 1H), 0.94-0.78 (m, 5H).

### Example 241

LC/MS: m/z=564.2 [M+H]⁺; rt: 2.26 min (LC/MS-method A). ¹H NMR (400 MHz, DMSO-d6) δ ppm: 10.79 (br s, 1H), 8.46 (d, J=7.9 Hz, 1H), 8.31 (d, J=5.3 Hz, 1H), 7.97 (br s, 1H), 7.60 (d, J=2.4 Hz, 1H), 7.50 (d, J=2.0 Hz, 1H), 7.03 (dd, J=5.3, 1.6 Hz, 1H), 6.94 (d, J=2.0 Hz, 1H), 5.37 (hept, J=6.6 Hz, 1H), 4.98 (q, J=7.1 Hz, 1H), 4.53-4.45 (m, 1H), 4.45-4.34 (m, 1H), 3.75-3.67 (m, 1H, partially overlapping water), 3.08 (dd, J=10.0, 4.1 Hz, 1H), 1.88-1.74 (m, 2H), 1.74-1.63 (m, 2H), 1.62-1.54 (m, 1H), 1.45 (d, J=7.1 Hz, 3H), 1.39-1.16 (m, 8H), 1.14-1.00 (m, 1H), 0.94-0.78 (m, 5H).

### Example 242: N-((1S)-2-((4-(2-methoxy-1-(4,4,4-trifluorobutanamido)ethyl)pyridin-2-yl)amino)-1-((1r,4S)-4-methylcyclohexyl)-2-oxoethyl)-4-methyl-1,2,5-oxadiazole-3-carboxamide

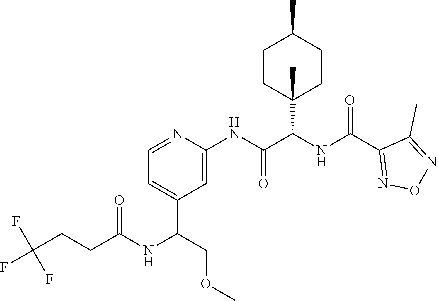

Following the procedure described for Example 39, using 35 mg (N-(1-(2-((S)-2-amino-2-((1r,4S)-4-methylcyclohexyl)acetamido)pyridin-4-yl)-2-methoxyethyl)-4,4,4-trifluorobutanamide (Intermediate 71), 35 mg of the title compound was obtained. LC/MS: m/z=555.2 [M+H]⁺; rt: 2.41 min (LC/MS-method A). ¹H NMR (400 MHz, DMSO-d₆) δ ppm: 10.78 (br s, 1H), 9.10 (d, J=8.1 Hz, 1H), 8.69 (d, J=7.9 Hz, 1H), 8.27 (d, J=5.2 Hz, 1H), 8.03 (br s, 1H), 7.11 (dd, J=5.2, 1.5 Hz, 1H), 5.02-4.93 (m, 1H), 4.63-4.55 (m, 1H), 3.59-3.47 (m, 2H), 3.25 (s, 3H), 2.55-2.40 (m, 7H, overlapping solvent), 1.89-1.75 (m, 2H), 1.74-1.65 (m, 2H), 1.64-1.56 (m, 1H), 1.37-1.17 (m, 2H), 1.158-1.01 (m, 1H), 0.95-0.79 (m, 5H).

Example 243 was prepared analogously to Example 242. Examples 244-246 were prepared analogously to Example 242 using the procedure described for Example 33.

| Ex. | Acid | Structure | Name | Yield |
|---|---|---|---|---|
| 243 | | | 4-cyclopropyl-N-((1S)-2-((4-(2- | 38 mg, |
| | | | methoxy-1-(4,4,4- | 83% |
| | | | trifluorobutanamido)ethyl)pyridin- | |
| | | | 2-yl)amino)-1-((1r,4S)-4- | |
| | | | methylcyclohexyl)-2-oxoethyl)- | |
| | | | 1,2,5-oxadiazole-3-carboxamide | |
| 244 | | | 1-ethyl-N-((1S)-2-((4-(2- | 43 mg, |
| | | | methoxy-1-(4,4,4- | 96% |
| | | | trifluorobutanamido)ethyl)pyridin- | |
| | | | 2-yl)amino)-1-((1r,4S)-4- | |
| | | | methylcyclohexyl)-2-oxoethyl)- | |
| | | | 1H-pyrazole-5-carboxamide | |
| 245 | | | N-((1S)-2-((4-(2-methoxy-1- | 43 mg, |
| | | | (4,4,4- | 99% |
| | | | trifluorobutanamido)ethyl)pyridin- | |
| | | | 2-yl)amino)-1-((1r,4S)-4- | |
| | | | methylcyclohexyl)-2-oxoethyl)-1- | |
| | | | methyl-1H-pyrazole-5- | |
| | | | carboxamide | |
| 246 | | | 1-isopropyl-N-((1S)-2-((4-(2- | 40 mg, |
| | | | methoxy-1-(4,4,4- | 87% |
| | | | trifluorobutanamido)ethyl)pyridin- | |
| | | | 2-yl)amino)-1-((1r,4S)-4- | |
| | | | methylcyclohexyl)-2-oxoethyl)- | |
| | | | 1H-pyrazole-5-carboxamide | |

### Example 243

LC/MS: m/z=581.2 [M+H]⁺; rt: 2.57 min (LC/MS-method A). ¹H NMR (400 MHz, DMSO-d6) δ ppm: 10.78 (s, 1H), 9.17 (d, J=8.0 Hz, 1H), 8.69 (d, J=7.9 Hz, 1H), 8.27 (d, J=5.2 Hz, 1H), 8.04 (br s, 1H), 7.10 (dd, J=5.2, 1.5 Hz, 1H), 5.02-4.94 (m, 1H), 4.65-4.57 (m, 1H), 3.59-3.47 (m, 2H), 3.25 (s, 3H), 2.55-2.40 (m, 4H, overlapping solvent), 2.27 (tt, J=8.3, 5.0 Hz, 1H), 1.87-1.75 (m, 2H), 1.74-1.65 (m, 2H), 1.64-1.56 (m, 1H), 1.36-1.18 (m, 2H), 1.17-1.03 (m, 3H), 1.01-0.80 (m, 7H).

### Example 244

LC/MS: m/z=567.2 [M+H]⁺; rt: 2.23 min (LC/MS-method A). ¹H NMR (400 MHz, DMSO-d6) δ ppm: 10.93 (br s, 1H), 8.73 (d, J=7.8 Hz, 1H), 8.52 (d, J=8.0 Hz, 1H), 8.28 (d, J=5.3 Hz, 1H), 8.00 (br s, 1H), 7.48 (d, J=2.1 Hz, 1H), 7.16 (dd, J=5.3, 1.6 Hz, 1H), 7.01 (d, J=2.1 Hz, 1H), 5.03-4.95 (m, 1H), 4.55-4.39 (m, 3H, partially overlapping water), 3.60-3.48 (m, 2H), 3.25 (s, 3H), 2.55-2.40 (m, 4H, overlapping solvent), 1.89-1.76 (m, 2H), 1.74-1.64 (m, 2H), 1.63-1.55 (m, 1H), 1.36-1.17 (m, 5H), 1.14-1.00 (m, 1H), 0.95-0.79 (m, 5H).

### Example 245

LC/MS: m/z=553.2 [M+H]⁺; rt: 2.13 min (LC/MS-method A). ¹H NMR (400 MHz, DMSO-d6) δ ppm: 10.92 (br s, 1H), 8.72 (d, J=7.9 Hz, 1H), 8.51 (d, J=7.9 Hz, 1H), 8.27 (d, J=5.4 Hz, 1H), 8.00 (br s, 1H), 7.46 (d, J=2.1 Hz, 1H), 7.15 (dd, J=5.4, 1.6 Hz, 1H), 7.04 (d, J=2.1 Hz, 1H), 5.03-4.95 (m, 1H), 4.52-4.44 (m, 1H), 4.01 (s, 3H, overlapping water), 3.59-3.48 (m, 2H), 3.25 (s, 3H), 2.55-2.40 (m, 4H, overlapping solvent), 1.90-1.75 (m, 2H), 1.74-1.63 (m, 2H), 1.63-1.55 (m, 1H), 1.36-1.17 (m, 2H), 1.13-0.99 (m, 1H), 0.95-0.79 (m, 5H).

### Example 246

LC/MS: m/z=581.2 [M+H]⁺; rt: 2.35 min (LC/MS-method A). ¹H NMR (400 MHz, DMSO-d6) δ ppm: 10.81 (br s, 1H), 8.71 (d, J=7.8 Hz, 1H), 8.48 (d, J=7.9 Hz, 1H), 8.27 (d, J=5.3 Hz, 1H), 8.01 (br s, 1H), 7.50 (d, J=2.0 Hz, 1H), 7.13 (dd, J=5.3, 1.6 Hz, 1H), 6.94 (d, J=2.0 Hz, 1H), 5.36 (hept, J=6.6 Hz, 1H), 5.02-4.94 (m, 1H), 4.51-4.43 (m, 1H), 3.59-3.48 (m, 2H), 3.25 (s, 3H), 2.55-2.40 (m, 4H, overlapping solvent), 1.89-1.75 (m, 2H), 1.75-1.63 (m, 2H), 1.62-1.55 (m, 1H), 1.40-1.17 (m, 8H), 1.14-1.01 (m, 1H), 0.95-0.79 (m, 5H).

### Example 247: 2-(3-Cyanophenyl)-2,2-difluoro-N—((S)-2-((4-((S)-2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)acetamide

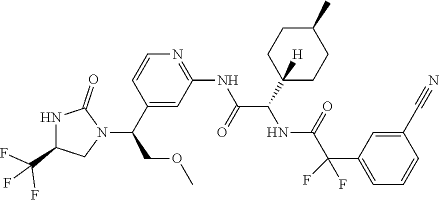

To a mixture of (2S)-2-amino-N-(4-((R or S)-2-methoxy-1-((S)-2-oxo-4-(trifluoromethyl)imidazolidin-1-yl)ethyl)pyridin-2-yl)-2-((1r,4S)-4-methylcyclohexyl)-acetamide hydrochloride salt (Intermediate 18-DS2, 20 mg), lithium 2-(3-cyanophenyl)-2,2-difluoroacetate (9 mg) and DCM (3 ml) DIPEA (26 μl) and T3P (79 μl) were added. After 1.5 h the mixture was concentrated in vacuo and the residue was purified by preparative RP HPLC (Purosphere® STAR-RP18, 25 mm×250 mm, 10 μm; 30 ml/min, from 95% H₂O/5% ACN to 10% H₂O/90% ACN in 45 min). The pure product containing fractions were combined, the ACN was partly removed and the residue freeze dried to yield 14 mg of the title compound. The absolute stereochemistry for the methoxy group was tentatively assigned. LC/MS: m/z=637.3 [M+H]⁺; rt: 2.37 min (LC/MS-method A). ¹H NMR (400 MHz, DMSO-d6) δ ppm: 10.67 (s, 1H), 9.08 (d, J=8.0 Hz, 1H), 8.29 (d, J=5.2 Hz, 1H), 8.15-8.12 (m, 1H), 8.07-8.03 (m, 1H), 7.98-7.92 (m, 2H), 7.78-7.72 (m, 1H), 7.63 (d, J=2.3 Hz, 1H), 6.99 (dd, J=5.2, 1.6 Hz, 1H), 5.01 (dd, J=8.2, 5.1 Hz, 1H), 4.49-4.36 (m, 2H), 3.83 (dd, J=10.5, 8.2 Hz, 1H), 3.78-3.70 (m, 2H), 3.31 (s, 4H), 1.85-1.74 (m, 1H), 1.70-1.59 (m, 3H), 1.55-1.47 (m, 1H), 1.29-1.07 (m, 2H), 1.02-0.75 (m, 6H).

Following the procedure described in Example 247 the following Examples were prepared:

| Ex. | Acid | Structure | Name | Yield |
|---|---|---|---|---|
| 248 | | | 2,2-difluoro-N-((S)-2-((4- | 14 mg, |
| | | | ((S)-2-methoxy-1-((S)-2- | 62% |
| | | | oxo-4-(trifluoromethyl)- | |
| | | | imidazolidin-1- | |
| | | | yl)ethyl)pyridin-2- | |
| | | | yl)amino)-1-((1r,4S)-4- | |
| | | | methylcyclohexyl)-2- | |
| | | | oxoethyl)-2-(1-methyl- | |
| | | | 1H-pyrazol-5- | |
| | | | yl)acetamide | |
| 249 | | | 2-(3-chlorophenyl)-2,2- | 17 mg, |
| | | | difluoro-N-((S)-2-((4-((S)- | 70% |
| | | | 2-methoxy-1-((S)-2-oxo- | |
| | | | 4-(trifluoromethyl)- | |
| | | | imidazolidin-1- | |
| | | | yl)ethyl)pyridin-2- | |
| | | | yl)amino)-1-((1r,4S)-4- | |
| | | | methylcyclohexyl)-2- | |
| | | | oxoethyl)acetamide | |
| 250 | | | 2-(3,5-difluorophenyl)- | 18 mg, |
| | | | 2,2-difluoro-N-((S)-2-((4- | 74% |
| | | | ((S)-2-methoxy-1-((S)-2- | |
| | | | oxo-4- | |
| | | | (trifluoromethyl)imidazolidin- | |
| | | | 1-yl)ethyl)pyridin-2- | |
| | | | yl)amino)-1-((1r,4S)-4- | |
| | | | methylcyclohexyl)-2- | |
| | | | oxoethyl)acetamide | |
| 251 | | | 2,2-difluoro-N-((S)-2-((4- | 16 mg, |
| | | | ((S)-2-methoxy-1-((S)-2- | 69% |
| | | | oxo-4- | |
| | | | (trifluoromethyl)imidazolidin- | |
| | | | 1-yl)ethyl)pyridin-2- | |
| | | | yl)amino)-1-((1r,4S)-4- | |
| | | | methylcyclohexyl)-2- | |
| | | | oxoethyl)-2-(pyrazin-2- | |
| | | | yl)acetamide | |
| 252 | | | 2,2-difluoro-N-((S)-2-((4- | 15 mg, |
| | | | ((S)-2-methoxy-1-((S)-2- | 61% |
| | | | oxo-4- | |
| | | | (trifluoromethyl)imidazolidin- | |
| | | | 1-yl)ethyl)pyridin-2- | |
| | | | yl)amino)-1-((1r,4S)-4- | |
| | | | methylcyclohexyl)-2- | |
| | | | oxoethyl)-2-(6- | |
| | | | methoxypyridin-3- | |
| | | | yl)acetamide | |
| 253 | | | (S)-N-(4-((S)-2-methoxy- | 12 mg, |
| | | | 1-((S)-2-oxo-4- | 53% |
| | | | (trifluoromethyl)imidazolidin- | |
| | | | 1-yl)ethyl)pyridin-2- | |
| | | | yl)-2-((1r,4S)-4- | |
| | | | methylcyclohexyl)-2-(2- | |
| | | | (5-methylisoxazol-3- | |
| | | | yl)acetamido)acetamide | |
| 254 | | | 2-fluoro-N-((S)-2-((4- | 18 mg, |
| | | | ((S)-2-methoxy-1-((S)-2- | 81% |
| | | | oxo-4-(trifluoromethyl)- | |
| | | | imidazolidin-1- | |
| | | | yl)ethyl)pyridin-2- | |
| | | | yl)amino)-1-((1r,4S)-4- | |
| | | | methylcyclohexyl)-2- | |
| | | | oxoethyl)-2- | |
| | | | phenylacetamide | |
| 255 | | | 2,2-difluoro-N-((S)-2-((4- | 7 mg, |
| | | | ((S)-2-methoxy-1-((S)-2- | 31% |
| | | | oxo-4-(trifluoromethyl)- | |
| | | | imidazolidin-1-yl)ethyl)- | |
| | | | pyridin-2-yl)amino)-1- | |
| | | | ((1r,4S)-4-methylcyclo- | |
| | | | hexyl)-2-oxo ethyl)-3- | |
| | | | methylbutanamide | |

### Example 248

LC/MS: m/z=616.3 [M+H]⁺; rt: 2.21 min (LC/MS-method A). ¹H NMR (400 MHz, DMSO-d6) δ ppm: 10.69 (s, 1H), 9.16 (d, J=7.5 Hz, 1H), 8.29 (d, J=5.2 Hz, 1H), 7.99 (br s, 1H), 7.64 (d, J=2.3 Hz, 1H), 7.49 (d, J=2.0 Hz, 1H), 7.01 (dd, J=5.2, 1.6 Hz, 1H), 6.57 (d, J=2.0 Hz, 1H), 5.02 (dd, J=8.2, 5.0 Hz, 1H), 4.50-4.37 (m, 2H), 3.93 (s, 3H), 3.83 (dd, J=10.6, 8.2 Hz, 1H), 3.79-3.71 (m, 2H), 3.36-3.28 (m, 4H, overlapping water), 1.86-1.61 (m, 4H), 1.57-1.48 (m, 1H), 1.33-1.13 (m, 2H), 1.07-0.92 (m, 1H), 0.90-0.76 (m, 5H).

### Example 249

LC/MS: m/z=646.2 [M+H]⁺; rt: 2.58 min (LC/MS-method A). ¹H NMR (400 MHz, DMSO-d6) δ ppm: 10.66 (s, 1H), 9.05 (d, J=8.0 Hz, 1H), 8.29 (d, J=5.3 Hz, 1H), 7.98 (br s, 1H), 7.71-7.68 (m, 1H), 7.66-7.53 (m, 4H), 6.99 (dd, J=5.3, 1.4 Hz, 1H), 5.01 (dd, J=8.2, 5.1 Hz, 1H), 4.49-4.35 (m, 2H), 3.83 (dd, J=10.5, 8.2 Hz, 1H), 3.78-3.70 (m, 2H), 3.33-3.25 (m, 4H, overlapping water), 1.86-1.74 (m, 1H), 1.72-1.59 (m, 3H), 1.54-1.47 (m, 1H), 1.30-1.07 (m, 2H), 1.01-0.75 (m, 6H).

### Example 250

LC/MS: m/z=648.2 [M+H]⁺; rt: 2.53 min (LC/MS-method A). ¹H NMR (400 MHz, DMSO-d6) δ ppm: 10.67 (s, 1H), 9.07 (d, J=7.9 Hz, 1H), 8.29 (d, J=5.2 Hz, 1H), 7.97 br (s, 1H), 7.63 (d, J=2.3 Hz, 1H), 7.51 (tt, J=9.2, 2.3 Hz, 1H), 7.43-7.35 (m, 2H), 6.99 (dd, J=5.2, 1.2 Hz, 1H), 5.01 (dd, J=8.1, 5.1 Hz, 1H), 4.49-4.36 (m, 2H), 3.83 (dd, J=10.6, 8.1 Hz, 1H), 3.78-3.70 (m, 2H), 3.33-3.24 (m, 4H, overlapping water), 1.85-1.74 (m, 1H), 1.71-1.59 (m, 3H), 1.55-1.47 (m, 1H), 1.30-1.07 (m, 2H), 1.03-0.75 (m, 6H).

### Example 251

LC/MS: m/z=614.2 [M+H]⁺; rt: 2.13 min (LC/MS-method A). ¹H NMR (400 MHz, DMSO-d6) δ ppm: 10.61 (s, 1H), 9.09-9.04 (m, 2H), 8.89 (d, J=2.5 Hz, 1H), 8.79 (dd, J=2.5, 1.5 Hz, 1H), 8.30 (d, J=5.2 Hz, 1H), 7.99 (br s, 1H), 7.63 (d, J=2.3 Hz, 1H), 7.01 (dd, J=5.2, 1.6 Hz, 1H), 5.01 (dd, J=8.2, 5.1 Hz, 1H), 4.50-4.39 (m, 2H), 3.83 (dd, J=10.5, 8.2 Hz, 1H), 3.79-3.71 (m, 2H), 3.35-3.26 (m, 4H, overlapping water), 1.86-1.60 (m, 4H), 1.58-1.48 (m, 1H), 1.32-1.10 (m, 2H), 1.05-0.76 (m, 6H).

### Example 252

LC/MS: m/z=643.3 [M+H]⁺; rt: 2.38 min (LC/MS-method A). ¹H NMR (400 MHz, DMSO-d6) δ ppm: 10.66 (s, 1H), 9.01 (d, J=7.9 Hz, 1H), 8.41 (d, J=2.6 Hz, 1H), 8.29 (d, J=5.2 Hz, 1H), 7.97 (br s, 1H), 7.91 (dd, J=8.8, 2.6 Hz, 1H), 7.63 (d, J=2.3 Hz, 1H), 7.00 (dd, J=5.2, 1.3 Hz, 1H), 6.95 (d, J=8.8 Hz, 1H), 5.01 (dd, J=8.1, 5.0 Hz, 1H), 4.49-4.35 (m, 2H), 3.89 (s, 3H), 3.83 (dd, J=10.5, 8.1 Hz, 1H), 3.78-3.70 (m, 2H), 3.34-3.25 (m, 4H, overlapping water), 1.86-1.59 (m, 4H), 1.55-1.46 (m, 1H), 1.30-1.09 (m, 2H), 1.03-0.76 (m, 6H).

### Example 253

LC/MS: m/z=581.3 [M+H]⁺; rt: 2.03 min (LC/MS-method A). ¹H NMR (400 MHz, DMSO-d6) δ ppm: 10.54 (s, 1H), 8.34 (d, J=8.2 Hz, 1H), 8.28 (d, J=5.2 Hz, 1H), 7.99 (br s, 1H), 7.62 (d, J=2.3 Hz, 1H), 6.98 (dd, J=5.2, 1.6 Hz, 1H), 6.12-6.10 (m, 1H), 5.01 (dd, J=8.2, 5.1 Hz, 1H), 4.49-4.39 (m, 2H), 3.83 (dd, J=10.5, 8.2 Hz, 1H), 3.78-3.70 (m, 2H), 3.61-3.52 (m, 2H), 3.34-3.25 (m, 4H, overlapping water), 2.35 (d, J=1.0 Hz, 3H), 1.77-1.50 (m, 5H), 1.32-0.98 (m, 3H), 0.91-0.78 (m, 5H).

### Example 254

LC/MS: m/z=594.3 [M+H]⁺; rt: 2.36 min (LC/MS-method A). ¹H NMR (400 MHz, DMSO-d6) δ ppm: 10.63-10.60 (m, 1H), 8.49-8.39 (m, 1H), 8.31-8.26 (m, 1H), 8.02-7.95 (m, 1H), 7.65-7.61 (m, 1H), 7.51-7.36 (m, 5H), 6.99 (d, J=5.0 Hz, 1H), 6.09-5.92 (m, 1H), 5.04-4.98 (m, 1H), 4.52-4.39 (m, 2H), 3.87-3.70 (m, 3H), 3.36-3.25 (m, 4H, overlapping water), 1.81-1.45 (m, 5H), 1.31-0.74 (m, 8H).

### Example 255

LC/MS: m/z=578.3 [M+H]⁺; rt: 2.41 min (LC/MS-method A). ¹H NMR (400 MHz, DMSO-d6) δ ppm: 10.61 (s, 1H), 8.59 (d, J=8.0 Hz, 1H), 8.29 (d, J=5.2 Hz, 1H), 7.98 (br s, 1H), 7.63 (d, J=2.3 Hz, 1H), 7.00 (dd, J=5.2, 1.5 Hz, 1H), 5.01 (dd, J=8.2, 5.0 Hz, 1H), 4.49-4.35 (m, 2H), 3.83 (dd, J=10.5, 8.2 Hz, 1H), 3.78-3.71 (m, 2H), 3.35-3.27 (m, 4H, overlapping water), 2.46-2.33 (m, 1H), 1.84-1.61 (m, 4H), 1.57-1.49 (m, 1H), 1.33-1.11 (m, 2H), 1.05-0.76 (m, 12H).

### Example 256: 4-methyl-N—((S)-1-((1r,4S)-4-methylcyclohexyl)-2-oxo-2-((4-((R)-1-((S)-2-oxo-4-(trifluoromethyl)imidazolidin-1-yl)propyl)pyridin-2-yl)amino)ethyl)-1,2,5-oxadiazole-3-carboxamide

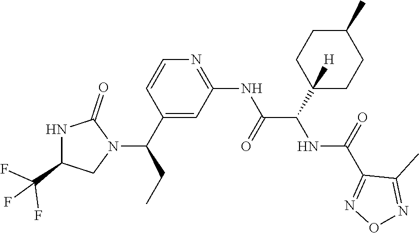

Following the synthesis route described for Intermediate 70, using ethylmagnesium bromide and the final step as described for Example 39 using 45 mg (S)-2-amino-2-((1r,4S)-4-methylcyclohexyl)-N-(4-((R)-1-((S)-2-oxo-4-(trifluoromethyl)-imidazolidin-1-yl)propyl)pyridin-2-yl)acetamide, 53 mg of the title compound was obtained. LC/MS: m/z=552.2 [M+H]⁺; rt: 2.37 min (LC/MS-method A). ¹H NMR (400 MHz, DMSO-d6) δ ppm: 10.76 (s, 1H), 9.08 (d, J=8.1 Hz, 1H), 8.30 (d, J=5.2 Hz, 1H), 8.00 (br s, 1H), 7.54 (d, J=2.4 Hz, 1H), 7.03 (dd, J=5.2, 1.6 Hz, 1H), 4.70 (dd, J=10.0, 5.8 Hz, 1H), 4.63-4.56 (m, 1H), 4.48-4.37 (m, 1H), 3.70-3.63 (m, 1H), 3.05 (dd, J=10.0, 4.5 Hz, 1H), 2.47 (s, 3H), 1.99-1.75 (m, 4H), 1.72-1.64 (m, 2H), 1.63-1.56 (m, 1H), 1.35-1.16 (m, 2H), 1.15-1.01 (m, 1H), 0.93-0.80 (m, 8H).

Example 257 was prepared analogously to Example 256. Examples 258-260 were prepared analogously to Example 256 using the procedure described for Example 33.

| Ex. | Acid | Structure | Name | Yield |
|---|---|---|---|---|
| 257 | | | 4-cyclopropyl-N-((S)-1- | 55 mg, |
| | | | ((1r,4S)-4- | 93% |
| | | | methylcyclohexyl)-2-oxo-2- | |
| | | | ((4-((R)-1-((S)-2-oxo-4- | |
| | | | (trifluoromethyl)imidazolidin- | |
| | | | 1-yl)propyl)pyridin-2- | |
| | | | yl)amino)ethyl)-1,2,5- | |
| | | | oxadiazole-3-carboxamide | |
| 258 | | | 1-ethyl-N-((S)-1-((1r,4S)-4- | 50 mg, |
| | | | methylcyclohexyl)-2-oxo-2- | 83% |
| | | | ((4-((R)-1-((S)-2-oxo-4- | |
| | | | (trifluoromethyl)imidazolidin- | |
| | | | 1-yl)propyl)pyridin-2- | |
| | | | yl)amino)ethyl)-1H- | |
| | | | pyrazole-5-carboxamide | |
| 259 | | | 1-methyl-N-((S)-1-((1r,4S)- | 53 mg, |
| | | | 4-methylcyclohexyl)-2-oxo- | 90% |
| | | | 2-((4-((R)-1-((S)-2-oxo-4- | |
| | | | (trifluoromethyl)imidazolidin- | |
| | | | 1-yl)propyl)pyridin-2- | |
| | | | yl)amino)ethyl)-1H- | |
| | | | pyrazole-5-carboxamide | |
| 260 | | | 1-isopropyl-N-((S)-1- | 51 mg, |
| | | | ((1r,4S)-4- | 83% |
| | | | methylcyclohexyl)-2-oxo-2- | |
| | | | ((4-((R)-1-((S)-2-oxo-4- | |
| | | | (trifluoromethyl)imidazolidin- | |
| | | | 1-yl)propyl)pyridin-2- | |
| | | | yl)amino)ethyl)-1H- | |
| | | | pyrazole-5-carboxamide | |

### Example 257

LC/MS: m/z=578.2 [M+H]⁺; rt: 2.54 min (LC/MS-method A). ¹H NMR (400 MHz, DMSO-d6) δ ppm: 10.77 (s, 1H), 9.16 (d, J=8.0 Hz, 1H), 8.31 (d, J=5.2 Hz, 1H), 8.00 (br s, 1H), 7.54 (d, J=2.4 Hz, 1H), 7.04 (dd, J=5.3, 1.6 Hz, 1H), 4.71 (dd, J=10.0, 5.8 Hz, 1H), 4.65-4.58 (m, 1H), 4.49-4.36 (m, 1H), 3.70-3.63 (m, 1H), 3.05 (dd, J=10.0, 4.5 Hz, 1H), 2.27 (tt, J=8.4, 5.0 Hz, 1H), 1.99-1.75 (m, 4H), 1.73-1.64 (m, 2H), 1.64-1.56 (m, 1H), 1.36-1.17 (m, 2H), 1.17-1.03 (m, 3H), 1.01-0.94 (m, 2H), 0.93-0.80 (m, 8H).

### Example 258

LC/MS: m/z=564.2 [M+H]⁺; rt: 2.24 min (LC/MS-method A). ¹H NMR (400 MHz, DMSO-d6) δ ppm: 10.84 (br s, 1H), 8.48 (d, J=7.9 Hz, 1H), 8.31 (d, J=5.3 Hz, 1H), 7.97 (br s, 1H), 7.56 (d, J=2.4 Hz, 1H), 7.48 (d, J=2.0 Hz, 1H), 7.06 (dd, J=5.3, 1.6 Hz, 1H), 7.01 (d, J=2.0 Hz, 1H), 4.71 (dd, J=10.0, 5.9 Hz, 1H), 4.53-4.39 (m, 4H, partially overlapping water), 3.71-3.62 (m, 1H), 3.06 (dd, J=10.0, 4.5 Hz, 1H), 1.99-1.75 (m, 4H), 1.73-1.64 (m, 2H), 1.62-1.54 (m, 1H), 1.35-1.16 (m, 5H), 1.14-0.99 (m, 1H), 0.94-0.79 (m, 8H).

### Example 259

LC/MS: m/z=550.2 [M+H]⁺; rt: 2.14 min (LC/MS-method A). ¹H NMR (400 MHz, DMSO-d6) δ ppm: 10.80 (br s, 1H), 8.47 (d, J=8.0 Hz, 1H), 8.30 (d, J=5.3 Hz, 1H), 7.98 (br s, 1H), 7.55 (d, J=2.4 Hz, 1H), 7.46 (d, J=2.1 Hz, 1H), 7.07-7.03 (m, 2H), 4.71 (dd, J=9.9, 5.9 Hz, 1H), 4.53-4.47 (m, 1H), 4.46-4.37 (m, 1H), 4.01 (s, 3H), 3.70-3.62 (m, 1H), 3.06 (dd, J=10.0, 4.5 Hz, 1H), 1.98-1.75 (m, 4H), 1.73-1.63 (m, 2H), 1.62-1.54 (m, 1H), 1.35-1.16 (m, 2H), 1.13-1.00 (m, 1H), 0.94-0.80 (m, 8H).

### Example 260

LC/MS: m/z=578.2 [M+H]⁺; rt: 2.36 min (LC/MS-method A). ¹H NMR (400 MHz, DMSO-d6) δ ppm: 10.80 (br s, 1H), 8.46 (d, J=7.9 Hz, 1H), 8.30 (d, J=5.4 Hz, 1H), 7.97 (br s, 1H), 7.55 (br d, J=2.4 Hz, 1H), 7.50 (d, J=2.0 Hz, 1H), 7.06 (dd, J=5.4, 1.6 Hz, 1H), 6.94 (d, J=2.0 Hz, 1H), 5.36 (hept, J=6.6 Hz, 1H), 4.71 (dd, J=9.9, 5.8 Hz, 1H), 4.52-4.37 (m, 2H), 3.71-3.62 (m, 1H), 3.06 (dd, J=10.0, 4.5 Hz, 1H), 1.99-1.75 (m, 4H), 1.73-1.63 (m, 2H), 1.63-1.54 (m, 1H), 1.39-1.16 (m, 8H), 1.14-1.00 (m, 1H), 0.94-0.79 (m, 8H).

### Example 261: N—((S)-1-(3,3-difluorocyclobutyl)-2-((4-((S)-2-methoxy-1-((S)-2-oxo-4-(trifluoromethyl)imidazolidin-1-yl)ethyl)pyridin-2-yl)amino)-2-oxoethyl)-4-ethylisoxazole-3-carboxamide

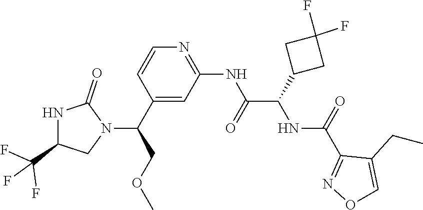 [SMILES: CCc1conc1C(=O)N[C@H](C(=O)Nc1cc([C@@H](COC)N2C[C@@H](C(F)(F)F)NC2=O)ccn1)C1CC(F)(F)C1]

Following the procedure described for Example 39 using 30 mg ((S)-2-amino-2-(3,3-difluorocyclobutyl)-N-(4-((S)-2-methoxy-1-((S)-2-oxo-4-(trifluoromethyl)imidazolidin-1-yl)ethyl)pyridin-2-yl)acetamide (Intermediate 72), 32 mg of the title compound was obtained. LC/MS: m/z=575.2 [M+H]⁺; rt: 1.99 min (LC/MS-method A). ¹H NMR (400 MHz, DMSO-d6) δ ppm: 10.77 (s, 1H), 8.99 (d, J=7.7 Hz, 1H), 8.88 (t, J=1.0 Hz, 1H), 8.31 (d, J=5.2 Hz, 1H), 7.97 (br s, 1H), 7.64 (d, J=2.4 Hz, 1H), 7.04 (dd, J=5.2, 1.6 Hz, 1H), 5.01 (dd, J=8.1, 5.0 Hz, 1H), 4.80-4.74 (m, 1H), 4.50-4.39 (m, 1H), 3.87-3.71 (m, 3H), 3.35-3.29 (m, 4H), 2.78-2.38 (m, 7H, overlapping solvent), 1.13 (t, J=7.5 Hz, 3H).

### Example 262: N—((S)-1-(3,3-difluorocyclobutyl)-2-((4-((S)-2-methoxy-1-((S)-2-oxo-4-(trifluoromethyl)imidazolidin-1-yl)ethyl)pyridin-2-yl)amino)-2-oxoethyl)-1-isopropyl-1H-pyrazole-5-carboxamide

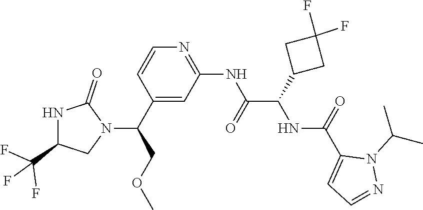 [SMILES: COC[C@H](c1ccnc(NC(=O)[C@@H](NC(=O)c2ccnn2C(C)C)C2CC(F)(F)C2)c1)N1C[C@@H](C(F)(F)F)NC1=O]

Following the procedure described for Example 33 using 25 mg ((S)-2-amino-2-(3,3-difluorocyclobutyl)-N-(4-((S)-2-methoxy-1-((S)-2-oxo-4-(trifluoromethyl)imidazolidin-1-yl)ethyl)pyridin-2-yl)acetamide (Intermediate 72), 30 mg of the title compound was obtained. LC/MS: m/z=588.2 [M+H]⁺; rt: 1.94 min (LC/MS-method A). ¹H NMR (400 MHz, DMSO-d6) δ ppm: 10.75 (s, 1H), 8.70 (d, J=7.5 Hz, 1H), 8.31 (d, J=5.3 Hz, 1H), 7.98 (br s, 1H), 7.63 (d, J=2.4 Hz, 1H), 7.51 (d, J=2.0 Hz, 1H), 7.03 (dd, J=5.3, 1.6 Hz, 1H), 6.92 (d, J=2.0 Hz, 1H), 5.38 (hept, J=6.5 Hz, 1H), 5.01 (dd, J=8.1, 5.0 Hz, 1H), 4.75-4.69 (m, 1H), 4.50-4.39 (m, 1H), 3.86-3.71 (m, 3H), 3.34-3.29 (m, 4H), 2.80-2.42 (m, 5H, overlapping solvent), 1.42-1.31 (m, 6H).

### Example 263: 4-Cyclopropyl-N—((S)-1-((1s,4R)-4-methylcyclohexyl)-2-oxo-2-((4-(((S)-2-oxo-4-(trifluoromethyl)imidazolidin-1-yl)methyl)pyridin-2-yl)amino)ethyl)-1,2,5-oxadiazole-3-carboxamide

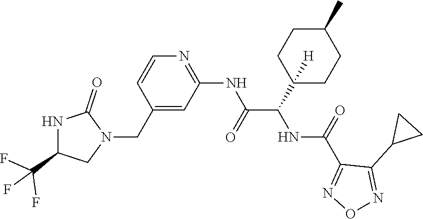

To a mixture of (S)-2-amino-2-((1s,4R)-4-methylcyclohexyl)-N-(4-(((S)-2-oxo-4-(trifluoromethyl)imidazolidin-1-yl)methyl)pyridin-2-yl)acetamide (Intermediate 58, 20 mg), 4-cyclopropyl-1,2,5-oxadiazole-3-carboxylic acid (8 mg) and DCM (1 ml) DIPEA (42 μl) and T3P (86 μl) were added. After 1 h the mixture was concentrated in vacuo and the residue was purified by preparative RP HPLC (Agilent Prep C18, 21.2 mm×250 mm, 10 μm; 50 ml/min, from 90% H₂O/10% ACN to 10% H₂O/90% ACN in 12.5 min). The pure product containing fractions were combined, the ACN was partly removed and the residue freeze dried to yield 17 mg of the title compound. LC/MS: m/z=550.1 [M+H]⁺; rt: 2.39 min (LC/MS-method A). ¹H NMR (400 MHz, DMSO-d6) δ ppm: 10.74 (s, 1H), 9.22 (d, J=8.3 Hz, 1H), 8.30 (d, J=5.1 Hz, 1H), 7.98 (br s, 1H), 7.65 (d, J=2.3 Hz, 1H), 6.96 (dd, J=5.1, 1.6 Hz, 1H), 4.82-4.75 (m, 1H), 4.46-4.36 (m, 2H), 4.22 (d, J=16.2 Hz, 1H), 3.62-3.55 (m, 1H), 3.34-3.29 (m, 1H, overlapping water), 2.27 (tt, J=8.4, 5.0 Hz, 1H), 2.02-1.93 (m, 1H), 1.72-1.63 (m, 1H), 1.54-1.30 (m, 8H), 1.16-1.10 (m, 2H), 1.00-0.95 (m, 2H), 0.92 (d, J=6.9 Hz, 3H).

### Example 264: 1-Methyl-N—((S)-1-((1s,4R)-4-methylcyclohexyl)-2-oxo-2-((4-(((S)-2-oxo-4-(trifluoromethyl)imidazolidin-1-yl)methyl)pyridin-2-yl)amino)ethyl)-1H-pyrazole-5-carboxamide

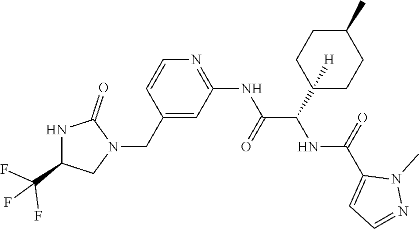

Following the procedure of Example 263, but using 1-methyl-1H-pyrazole-5-carboxylic acid (7 mg), 17 mg (73%) of the title compound was obtained. LC/MS: m/z=522.2 [M+H]⁺; rt: 1.97 min (LC/MS-method A). ¹H NMR (400 MHz, DMSO-d6) δ ppm: 10.62 (s, 1H), 8.47 (d, J=8.2 Hz, 1H), 8.28 (d, J=5.1 Hz, 1H), 7.99 (br s, 1H), 7.64 (d, J=2.4 Hz, 1H), 7.46 (d, J=2.1 Hz, 1H), 7.02 (d, J=2.1 Hz, 1H), 6.94 (dd, J=5.1, 1.5 Hz, 1H), 4.69 (dd, J=10.0, 8.2 Hz, 1H), 4.47-4.35 (m, 2H), 4.21 (d, J=16.2 Hz, 1H), 4.02 (s, 3H), 3.62-3.54 (m, 1H), 3.34-3.28 (m, 1H, overlapping water), 2.04-1.93 (m, 1H), 1.72-1.61 (m, 1H), 1.56-1.27 (m, 8H), 0.92 (d, J=6.8 Hz, 3H).

### Example 265: N—((S)-1-((4-((S)-2-methoxy-1-((S)-2-oxo-4-(trifluoromethyl)-imidazolidin-1-yl)ethyl)pyridin-2-yl)amino)-1-oxo-3-((1,1,1-trifluoro-2-methylpropan-2-yl)oxy)propan-2-yl)-4-methylisoxazole-3-carboxamide

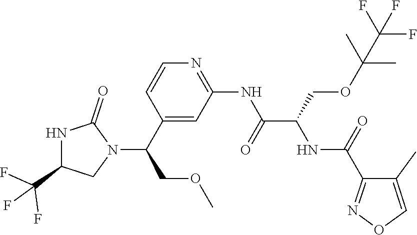 [SMILES: COC[C@H](c1ccnc(NC(=O)[C@H](COC(C)(C)C(F)(F)F)NC(=O)c2nocc2C)c1)N1C[C@@H](C(F)(F)F)NC1=O]

To a mixture of (S)-2-amino-N-(4-((S)-2-methoxy-1-((S)-2-oxo-4-(trifluoromethyl)imidazolidin-1-yl)ethyl)pyridin-2-yl)-3-((1,1,1-trifluoro-2-methylpropan-2-yl)oxy)propanamide hydrochloride (Intermediate 59, 20 mg), 4-methylisoxazole-3-carboxylic acid (5 mg) and DCM (1 ml) DIPEA (30 μl) and T3P (70 μl) were added. After 15 min the mixture was concentrated in vacuo and the residue was purified by preparative RP HPLC (Agilent Prep C18, 21.2 mm×250 mm, 10 μm; 50 ml/min, from 90% H₂O/10% ACN to 10% H₂O/90% ACN in 12.5 min). The pure product containing fractions were combined, the ACN was partly removed and the residue freeze dried to yield 14 mg of the title compound. LC/MS: m/z=611.1 [M+H]⁺; rt: 2.05 min (LC/MS-method A). ¹H NMR (400 MHz, DMSO-d6) δ ppm: 10.71 (s, 1H), 8.86 (q, J=1.1 Hz, 1H), 8.52 (d, J=7.8 Hz, 1H), 8.30 (d, J=5.2 Hz, 1H), 7.99 (br s, 1H), 7.63 (d, J=2.3 Hz, 1H), 7.01 (dd, J=5.2, 1.5 Hz, 1H), 5.01 (dd, J=8.1, 5.2 Hz, 1H), 4.92-4.85 (m, 1H), 4.49-4.38 (m, 1H), 3.95-3.86 (m, 2H), 3.83 (dd, J=10.5, 8.1 Hz, 1H), 3.78-3.71 (m, 2H), 3.34-3.25 (m, 4H, overlapping water), 2.12 (d, J=1.1 Hz, 3H), 1.32 (s, 3H), 1.30 (s, 3H).

Following the procedure described in Example 265 with reaction times ranging from 15 to 30 min the following examples were prepared:

| Ex. | Acid | Structure | Name | Yield |
|---|---|---|---|---|
| 266 | | | 4-ethyl-N-((S)-1-((4-((S)-2- | 12 mg, |
| | | | methoxy-1-((S)-2-oxo-4- | 51% |
| | | | (trifluoromethyl)imidazolidin-1- | |
| | | | yl)ethyl)pyridin-2-yl)amino)-1- | |
| | | | oxo-3-((1,1,1-trifluoro-2- | |
| | | | methylpropan-2-yl)oxy)propan-2- | |
| | | | yl)-1,2,5-oxadiazole-3- | |
| | | | carboxamide | |
| 267 | | | 3-ethyl-N-((S)-1-((4-((S)-2- | 12 mg, |
| | | | methoxy-1-((S)-2-oxo-4- | 51% |
| | | | (trifluoromethyl)imidazolidin-1- | |
| | | | yl)ethyl)pyridin-2-yl)amino)-1- | |
| | | | oxo-3-((1,1,1-trifluoro-2- | |
| | | | methylpropan-2-yl)oxy)propan-2- | |
| | | | yl)isoxazole-4-carboxamide | |
| 268 | | | N-((S)-1-((4-((S)-2-methoxy-1- | 8 mg, |
| | | | ((S)-2-oxo-4- | 57% |
| | | | (trifluoromethyl)imidazolidin-1- | |
| | | | yl)ethyl)pyridin-2-yl)amino)-1- | |
| | | | oxo-3-((1,1,1-trifluoro-2- | |
| | | | methylpropan-2-yl)oxy)propan-2- | |
| | | | yl)-4-methyl-1,2,5-oxadiazole-3- | |
| | | | carboxamide | |
| 269 | | | 4-cyclopropyl-N-((S)-1-((4-((S)-2- | 8 mg, |
| | | | methoxy-1-((S)-2-oxo-4- | 59% |
| | | | (trifluoromethyl)imidazolidin-1- | |
| | | | yl)ethyl)pyridin-2-yl)amino)-1- | |
| | | | oxo-3-((1,1,1-trifluoro-2- | |
| | | | methylpropan-2-yl)oxy)propan-2- | |
| | | | yl)-1,2,5-oxadiazole-3- | |
| | | | carboxamide | |
| 270 | | | 1-ethyl-N-((S)-1-((4-((S)-2- | 13 mg, |
| | | | methoxy-1-((S)-2-oxo-4- | 56% |
| | | | (trifluoromethyl)imidazolidin-1- | |
| | | | yl)ethyl)pyridin-2-yl)amino)-1- | |
| | | | oxo-3-((1,1,1-trifluoro-2- | |
| | | | methylpropan-2-yl)oxy)propan-2- | |
| | | | yl)-1H-pyrazole-5-carboxamide | |
| 271 | | | N-((S)-1-((4-((S)-2-methoxy-1- | 10 mg, |
| | | | ((S)-2-oxo-4- | 70% |
| | | | (trifluoromethyl)imidazolidin-1- | |
| | | | yl)ethyl)pyridin-2-yl)amino)-1- | |
| | | | oxo-3-((1,1,1-trifluoro-2- | |
| | | | methylpropan-2-yl)oxy)propan-2- | |
| | | | yl)-1-methyl-1H-pyrazole-5- | |
| | | | carboxamide | |
| 272 | | | 1-isopropyl-N-((S)-1-((4-((S)-2- | 13 mg, |
| | | | methoxy-1-((S)-2-oxo-4- | 56% |
| | | | (trifluoromethyl)imidazolidin-1- | |
| | | | yl)ethyl)pyridin-2-yl)amino)-1- | |
| | | | oxo-3-((1,1,1-trifluoro-2- | |
| | | | methylpropan-2-yl)oxy)propan-2- | |
| | | | yl)-1H-pyrazole-5-carboxamide | |

### Example 266

LC/MS: m/z=626.0 [M+H]⁺; rt: 2.25 min (LC/MS-method A). ¹H NMR (400 MHz, DMSO-d6) δ ppm: 10.77 (s, 1H), 9.15 (d, J=7.7 Hz, 1H), 8.31 (d, J=5.1 Hz, 1H), 7.99 (br s, 1H), 7.63 (d, J=2.3 Hz, 1H), 7.02 (dd, J=5.1, 1.5 Hz, 1H), 5.01 (dd, J=8.0, 5.2 Hz, 1H), 4.97-4.89 (m, 1H), 4.49-4.38 (m, 1H), 3.95-3.70 (m, 5H), 3.35-3.25 (m, 4H), 2.92 (q, J=7.5 Hz, 2H), 1.33 (s, 3H), 1.32 (s, 3H), 1.25 (t, J=7.5 Hz, 3H).

### Example 267

LC/MS: m/z=625.1 [M+H]⁺; rt: 2.04 min (LC/MS-method A). ¹H NMR (400 MHz, DMSO-d6) δ ppm: 10.69 (s, 1H), 9.37 (s, 1H), 8.55 (d, J=7.8 Hz, 1H), 8.30 (d, J=5.1 Hz, 1H), 8.01 (s, 1H), 7.62 (d, J=2.3 Hz, 1H), 7.00 (dd, J=5.1, 1.5 Hz, 1H), 5.01 (dd, J=8.0, 5.2 Hz, 1H), 4.93-4.85 (m, 1H), 4.49-4.38 (m, 1H), 3.87-3.70 (m, 5H), 3.30 (s, 3H, overlapping water), 3.26 (dd, J=10.0, 3.9 Hz, 1H), 2.84 (q, J=7.5 Hz, 2H), 1.32 (s, 6H), 1.18 (t, J=7.5 Hz, 3H).

### Example 268

LC/MS: m/z=612.0 [M+H]⁺; rt: 2.11 min (LC/MS-method A). ¹H NMR (400 MHz, DMSO-d6) δ ppm: 10.73 (s, 1H), 9.08 (d, J=7.8 Hz, 1H), 8.30 (d, J=5.2 Hz, 1H), 7.99 (br s, 1H), 7.60 (d, J=2.3 Hz, 1H), 7.02 (dd, J=5.2, 1.6 Hz, 1H), 5.01 (dd, J=8.1, 5.2 Hz, 1H), 4.95-4.90 (m, 1H), 4.47-4.39 (m, 1H), 3.94-3.87 (m, 2H), 3.82 (dd, J=10.6, 8.1 Hz, 1H), 3.78-3.72 (m, 2H), 3.31 (s, 3H), 3.29-3.26 (m, 1H, overlapping water), 2.49 (s, 3H), 1.33 (s, 3H), 1.32 (s, 3H).

### Example 269

LC/MS: m/z=638.1 [M+H]⁺; rt: 2.29 min (LC/MS-method A). ¹H NMR (400 MHz, DMSO-d6) δ ppm: 10.74 (s, 1H), 9.15 (d, J=7.7 Hz, 1H), 8.31 (d, J=5.2 Hz, 1H), 8.00 (br s, 1H), 7.60 (d, J=2.4 Hz, 1H), 7.02 (dd, J=5.2, 1.6 Hz, 1H), 5.01 (dd, J=8.1, 5.2 Hz, 1H), 4.97-4.92 (m, 1H), 4.47-4.39 (m, 1H), 3.94-3.88 (m, 2H), 3.83 (dd, J=10.5, 8.1 Hz, 1H), 3.78-3.73 (m, 2H), 3.31 (s, 3H), 3.30-3.26 (m, 1H, overlapping water), 2.33 (tt, J=8.4, 5.0 Hz, 1H), 1.33 (s, 3H), 1.32 (s, 3H), 1.16-1.11 (m, 2H), 1.02-0.97 (m, 2H).

### Example 270

LC/MS: m/z=624.1 [M+H]⁺; rt: 1.98 min (LC/MS-method A). ¹H NMR (400 MHz, DMSO-d6) δ ppm: 10.67 (s, 1H), 8.56 (d, J=7.8 Hz, 1H), 8.30 (d, J=5.3 Hz, 1H), 8.00 (br s, 1H), 7.62 (d, J=2.3 Hz, 1H), 7.49 (d, J=2.1 Hz, 1H), 7.01 (dd, J=5.3, 1.5 Hz, 1H), 6.93 (d, J=2.1 Hz, 1H), 5.01 (dd, J=8.0, 5.2 Hz, 1H), 4.91-4.84 (m, 1H), 4.51-4.38 (m, 3H), 3.92-3.70 (m, 5H), 3.32-3.24 (m, 4H, overlapping water), 1.33 (br s, 6H), 1.28 (t, J=7.2 Hz, 3H).

### Example 271

LC/MS: m/z=610.0 [M+H]⁺; rt: 1.87 min (LC/MS-method A). ¹H NMR (400 MHz, DMSO-d6) δ ppm: 10.63 (s, 1H), 8.54 (d, J=7.8 Hz, 1H), 8.30 (d, J=5.2 Hz, 1H), 8.00 (br s, 1H), 7.59 (d, J=2.4 Hz, 1H), 7.47 (d, J=2.1 Hz, 1H), 7.01 (dd, J=5.2, 1.6 Hz, 1H), 6.96 (d, J=2.1 Hz, 1H), 5.01 (dd, J=8.1, 5.2 Hz, 1H), 4.91-4.85 (m, 1H), 4.47-4.39 (m, 1H), 4.03 (s, 3H), 3.88 (dd, J=9.4, 5.8 Hz, 1H), 3.85-3.80 (m, 2H), 3.77-3.72 (m, 2H), 3.30 (s, 3H), 3.29-3.25 (m, 1H, overlapping water), 1.34-1.32 (m, 6H).

### Example 272

LC/MS: m/z=638.2 [M+H]⁺; rt: 2.10 min (LC/MS-method A). ¹H NMR (400 MHz, DMSO-d6) δ ppm: 10.67 (s, 1H), 8.55 (d, J=7.7 Hz, 1H), 8.30 (d, J=5.2 Hz, 1H), 8.00 (br s, 1H), 7.62 (d, J=2.3 Hz, 1H), 7.51 (d, J=2.0 Hz, 1H), 7.01 (dd, J=5.2, 1.4 Hz, 1H), 6.87 (d, J=2.0 Hz, 1H), 5.39 (hept, J=6.7 Hz, 1H), 5.01 (dd, J=8.0, 5.2 Hz, 1H), 4.91-4.83 (m, 1H), 4.49-4.38 (m, 1H), 3.92-3.70 (m, 5H), 3.32-3.24 (m, 4H, overlapping water), 1.39-1.31 (m, 12H).

### Example 273: 4-methyl-N—((S)-1-oxo-1-((4-(((S)-2-oxo-4-(trifluoromethyl)-imidazolidin-1-yl)methyl)pyridin-2-yl)amino)-3-((1,1,1-trifluoro-2-methylpropan-2-yl)oxy)propan-2-yl)-1,2,5-oxadiazole-3-carboxamide

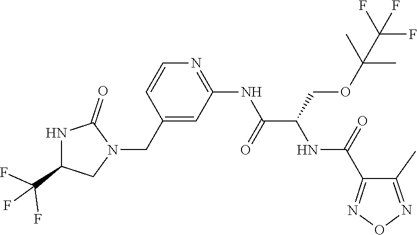 [SMILES: Cc1nonc1C(=O)N[C@@H](COC(C)(C)C(F)(F)F)C(=O)Nc1cc(CN2C[C@@H](C(F)(F)F)NC2=O)ccn1]

To a mixture of (S)-2-amino-N-(4-(((S)-2-oxo-4-(trifluoromethyl)imidazolidin-1-yl)methyl)pyridin-2-yl)-3-((1,1,1-trifluoro-2-methylpropan-2-yl)oxy)propenamide hydrochloride (Intermediate 60, 9 mg), 4-methyl-1,2,5-oxadiazole-3-carboxylic acid (3 mg) and DCM (0.8 ml) DIPEA (20 μl) and T3P (30 μl) were added. After 1 h the mixture was concentrated in vacuo and the residue was purified by preparative RP HPLC (Agilent Prep C18, 21.2 mm×250 mm, 10 μm; 50 ml/min, from 90% H₂O/10% ACN to 10% H₂O/90% ACN in 12.5 min). The pure product containing fractions were combined, the ACN was partly removed and the residue freeze dried to yield 5 mg of the title compound. LC/MS: m/z=568.2 [M+H]⁺; rt: 2.07 min (LC/MS-method A). ¹H NMR (400 MHz, DMSO-d6) δ ppm: 10.73 (s, 1H), 9.07 (d, J=7.8 Hz, 1H), 8.30 (d, J=5.1 Hz, 1H), 7.96 (br s, 1H), 7.62 (d, J=2.4 Hz, 1H), 6.97 (dd, J=5.1, 1.5 Hz, 1H), 4.95-4.90 (m, 1H), 4.45-4.37 (m, 2H), 4.22 (d, J=16.1 Hz, 1H), 3.94-3.87 (m, 2H), 3.61-3.56 (m, 1H), 3.31 (dd, J=10.0, 4.2 Hz, 1H), 2.49 (s, 3H), 1.33 (s, 3H), 1.32 (s, 3H).

Following the procedure described in Example 273 with reaction times ranging from 15 to 30 min the following examples were prepared:

| Ex. | Acid | Structure | Name | Yield |
|---|---|---|---|---|
| 274 | | | 4-cyclopropyl-N-((S)-1-oxo- | 5 mg, |
| | | | 1-((4-(((S)-2-oxo-4- | 42% |
| | | | (trifluoromethyl)imidazolidin- | |
| | | | 1-yl)methyl)pyridin-2- | |
| | | | yl)amino)-3-((1,1,1-trifluoro- | |
| | | | 2-methylpropan-2- | |
| | | | yl)oxy)propan-2-yl)-1,2,5- | |
| | | | oxadiazole-3-carboxamide | |
| 275 | | | 1-methyl-N-((S)-1-oxo-1-((4- | 7 mg, |
| | | | (((S)-2-oxo-4- | 67% |
| | | | (trifluoromethyl)imidazolidin- | |
| | | | 1-yl)methyl)pyridin-2- | |
| | | | yl)amino)-3-((1,1,1-trifluoro- | |
| | | | 2-methylpropan-2- | |
| | | | yl)oxy)propan-2-yl)-1H- | |
| | | | pyrazole-5-carboxamide | |

### Example 274

LC/MS: m/z=594.1 [M+H]⁺; rt: 2.26 min (LC/MS-method A). ¹H NMR (400 MHz, DMSO-d6) δ ppm: 10.74 (s, 1H), 9.14 (d, J=7.7 Hz, 1H), 8.30 (d, J=5.1 Hz, 1H), 7.96 (br s, 1H), 7.62 (d, J=2.4 Hz, 1H), 6.97 (dd, J=5.1, 1.5 Hz, 1H), 4.97-4.91 (m, 1H), 4.44-4.37 (m, 2H), 4.22 (d, J=16.1 Hz, 1H), 3.94-3.88 (m, 2H), 3.61-3.56 (m, 1H), 3.31 (dd, J=10.0, 4.2 Hz, 1H), 2.33 (tt, J=8.4, 5.0 Hz, 1H), 1.33 (s, 3H), 1.32 (s, 3H), 1.16-1.12 (m, 2H), 1.02-0.97 (m, 2H).

### Example 275

LC/MS: m/z=566.1 [M+H]⁺; rt: 1.82 min (LC/MS-method A). ¹H NMR (400 MHz, DMSO-d6) δ ppm: 10.62 (s, 1H), 8.53 (d, J=7.8 Hz, 1H), 8.29 (d, J=5.1 Hz, 1H), 7.96 (br s, 1H), 7.61 (d, J=2.4 Hz, 1H), 7.47 (d, J=2.0 Hz, 1H), 6.97-6.95 (m, 2H), 4.90-4.85 (m, 1H), 4.44-4.37 (m, 2H), 4.21 (d, J=16.1 Hz, 1H), 4.03 (s, 3H), 3.89 (dd, J=9.4, 5.8 Hz, 1H), 3.83 (dd, J=9.4, 7.2 Hz, 1H), 3.61-3.55 (m, 1H), 3.30 (dd, J=10.0, 4.2 Hz, 1H), 1.34-1.32 (m, 6H).

### Example 276: 4-Cyclopropyl-N-(1-(4-fluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-1,2,5-oxadiazole-3-carboxamide—Diastereomer 1

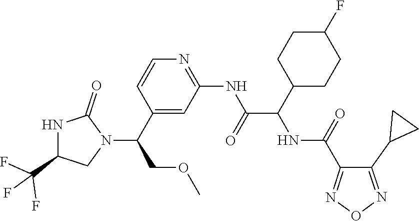 [SMILES: COC[C@H](c1ccnc(NC(=O)C(NC(=O)c2nonc2C2CC2)C2CCC(F)CC2)c1)N1C[C@@H](C(F)(F)F)NC1=O]

To a mixture of 2-amino-2-(4-fluorocyclohexyl)-N-(4-((S)-2-methoxy-1-((S)-2-oxo-4-(trifluoromethyl)imidazolidin-1-yl)ethyl)pyridin-2-yl)acetamide (Intermediate 62-DS1, 20 mg), 4-cyclopropyl-1,2,5-oxadiazole-3-carboxylic acid (8 mg) and DCM (1.0 ml) DIPEA (40 μl) and T3P (80 μl) were added. After 20 min the mixture was concentrated in vacuo and the residue was purified by preparative RP HPLC (Agilent Prep C18, 21.2 mm×250 mm, 10 μm; 50 ml/min, from 90% H₂O/10% ACN to 10% H₂O/90% ACN in 12.5 min). The pure product containing fractions were combined, the ACN was partly removed and the residue freeze dried to yield 18 mg of the title compound. LC/MS: m/z=598.2 [M+H]⁺; rt: 2.14 min (LC/MS-method A). ¹H NMR (400 MHz, DMSO-d6) δ ppm: 10.76 (s, 1H), 9.21 (d, J=8.0 Hz, 1H), 8.31 (d, J=5.3 Hz, 1H), 8.01 (br s, 1H), 7.63 (d, J=2.3 Hz, 1H), 7.02 (dd, J=5.3, 1.5 Hz, 1H), 5.01 (dd, J=8.2, 5.0 Hz, 1H), 4.69-4.62 (m, 1H), 4.61-4.38 (m, 2H), 3.83 (dd, J=10.5, 8.2 Hz, 1H), 3.79-3.71 (m, 2H), 3.33-3.27 (m, 4H, overlapping water), 2.27 (tt, J=8.4, 5.0 Hz, 1H), 2.11-2.00 (m, 2H), 1.94-1.81 (m, 2H), 1.72-1.63 (m, 1H), 1.47-1.08 (m, 6H), 1.00-0.94 (m, 2H).

The following diastereomers we synthesised as described in Example 276:

| Ex. | Amine | Structure | Name | Yield |
|---|---|---|---|---|
| 277 | I-062-DS2 | diastereomer 2 | 4-Cyclopropyl-N-(1-(4- | 19 mg, |
| | | | fluorocyclohexyl)-2-((4-((S)- | 75% |
| | | | 2-methoxy-1-((S)-2-oxo-4- | |
| | | | (trifluoromethyl)imidazolidin- | |
| | | | 1-yl)ethyl)pyridin-2- | |
| | | | yl)amino)-2-oxoethyl)-1,2,5- | |
| | | | oxadiazole-3-carboxamide- | |
| | | | diastereomer 2 | |
| 278 | I-062-DS3 | diastereomer 3 | 4-Cyclopropyl-N-(1-(4- | 19 mg, |
| | | | fluorocyclohexyl)-2-((4-((S)- | 71% |
| | | | 2-methoxy-1-((S)-2-oxo-4- | |
| | | | (trifluoromethyl)imidazolidin- | |
| | | | 1-yl)ethyl)pyridin-2- | |
| | | | yl)amino)-2-oxoethyl)-1,2,5- | |
| | | | oxadiazole-3-carboxamide- | |
| | | | diastereomer 3 | |
| 279 | I-062-DS4 | diastereomer 4 | 4-Cyclopropyl-N-(1-(4- | 19 mg, |
| | | | fluorocyclohexyl)-2-((4-((S)- | 72% |
| | | | 2-methoxy-1-((S)-2-oxo-4- | |
| | | | (trifluoromethyl)imidazolidin- | |
| | | | 1-yl)ethyl)pyridin-2- | |
| | | | yl)amino)-2-oxoethyl)-1,2,5- | |
| | | | oxadiazole-3-carboxamide- | |
| | | | diastereomer 4 | |

### Example 277

LC/MS: m/z=598.2 [M+H]⁺; rt: 2.11 min (LC/MS-method A). ¹H NMR (400 MHz, DMSO-d6) δ ppm: 10.78 (s, 1H), 9.23 (d, J=8.0 Hz, 1H), 8.30 (d, J=5.2 Hz, 1H), 8.02 (br s, 1H), 7.63 (d, J=2.3 Hz, 1H), 7.01 (dd, J=5.2, 1.5 Hz, 1H), 5.01 (dd, J=8.2, 5.0 Hz, 1H), 4.81 (br d, J=48.7 Hz, 1H), 4.72-4.65 (m, 1H), 4.49-4.39 (m, 1H), 3.83 (dd, J=10.5, 8.2 Hz, 1H), 3.79-3.71 (m, 2H), 3.33-3.27 (m, 4H, overlapping water), 2.27 (tt, J=8.4, 5.0 Hz, 1H), 2.01-1.86 (m, 3H), 1.70-1.31 (m, 6H), 1.16-1.09 (m, 2H), 1.01-0.95 (m, 2H).

### Example 278

LC/MS: m/z=598.2 [M+H]⁺; rt: 2.14 min (LC/MS-method A). ¹H NMR (400 MHz, DMSO-d6) δ ppm: 10.75 (s, 1H), 9.21 (d, J=8.0 Hz, 1H), 8.30 (d, J=5.2 Hz, 1H), 8.01 (br s, 1H), 7.63 (d, J=2.3 Hz, 1H), 7.02 (dd, J=5.2, 1.5 Hz, 1H), 5.01 (dd, J=8.2, 5.1 Hz, 1H), 4.68-4.62 (m, 1H), 4.60-4.39 (m, 2H), 3.86-3.71 (m, 3H), 3.37-3.29 (m, 4H, overlapping water), 2.28 (tt, J=8.3, 5.0 Hz, 1H), 2.11-2.00 (m, 2H), 1.94-1.81 (m, 2H), 1.72-1.63 (m, 1H), 1.46-1.07 (m, 6H), 1.01-0.94 (m, 2H).

### Example 279

LC/MS: m/z=598.2 [M+H]⁺; rt: 2.12 min (LC/MS-method A). ¹H NMR (400 MHz, DMSO-d6) δ ppm: 10.76 (s, 1H), 9.23 (d, J=8.1 Hz, 1H), 8.30 (d, J=5.2 Hz, 1H), 8.03 (br s, 1H), 7.63 (d, J=2.4 Hz, 1H), 7.02 (dd, J=5.2, 1.6 Hz, 1H), 5.01 (dd, J=8.2, 5.1 Hz, 1H), 4.81 (d, J=48.8 Hz, 1H), 4.72-4.65 (m, 1H), 4.50-4.39 (m, 1H), 3.86-3.71 (m, 3H), 3.36-3.30 (m, 4H, overlapping water), 2.27 (tt, J=8.4, 5.0 Hz, 1H), 2.01-1.87 (m, 3H), 1.69-1.32 (m, 6H), 1.16-1.09 (m, 2H), 1.01-0.94 (m, 2H).

Following the procedure described in Example 276 with reaction times ranging from 0.20 min to 2 h the following examples were prepared using Intermediate 62-DS1:

| Ex. | Acid | Structure | Name | Yield |
|---|---|---|---|---|
| 280 | | diastereomer 1 | 1-ethyl-N-(1-(4- | 15 mg, |
| | | | fluorocyclohexyl)-2-((4-((S)-2- | 62% |
| | | | methoxy-1-((S)-2-oxo-4- | |
| | | | (trifluoromethyl)imidazolidin- | |
| | | | 1-yl)ethyl)pyridin-2-yl)amino)- | |
| | | | 2-oxoethyl)-1H-pyrazole-5- | |
| | | | carboxamide-diastereomer 1 | |
| 281 | | diastereomer 1 | 4-(2,2-difluoroethoxy)-N-(1- | 19 mg, |
| | | | (4-fluorocyclohexyl)-2-((4- | 73% |
| | | | ((S)-2-methoxy-1-((S)-2-oxo- | |
| | | | 4-(trifluoromethyl) | |
| | | | imidazolidin-1- | |
| | | | yl)ethyl)pyridin-2-yl)amino)-2- | |
| | | | oxoethyl)isoxazole-3- | |
| | | | carboxamide-diastereomer 1 | |
| 282 | | diastereomer 1 | N-(1-(4-fluorocyclohexyl)-2- | 14 mg, |
| | | | ((4-((S)-2-methoxy-1-((S)-2- | 51% |
| | | | oxo-4-(trifluoromethyl) | |
| | | | imidazolidin-1- | |
| | | | yl)ethyl)pyridin-2-yl)amino)-2- | |
| | | | oxoethyl)-4-(2,2,2- | |
| | | | trifluoroethoxy)-1,2,5- | |
| | | | oxadiazole-3-carboxamide- | |
| | | | diastereomer 1 | |
| 283 | | diastereomer 1 | N-(1-(4-fluorocyclohexyl)-2- | 13 mg, |
| | | | ((4-((S)-2-methoxy-1-((S)-2- | 57% |
| | | | oxo-4-(trifluoromethyl) | |
| | | | imidazolidin-1- | |
| | | | yl)ethyl)pyridin-2-yl)amino)-2- | |
| | | | oxoethyl)-3-methylisoxazole- | |
| | | | 4-carboxamide-diastereomer 1 | |
| 284 | | diastereomer 1 | N-(1-(4-fluorocyclohexyl)-2- | 16 mg, |
| | | | ((4-((S)-2-methoxy-1-((S)-2- | 66% |
| | | | oxo-4-(trifluoromethyl) | |
| | | | imidazolidin-1- | |
| | | | yl)ethyl)pyridin-2-yl)amino)-2- | |
| | | | oxoethyl)-4-methylisoxazole- | |
| | | | 3-carboxamide-diastereomer 1 | |
| 285 | | diastereomer 1 | 4-ethyl-N-(1-(4- | 14 mg, |
| | | | fluorocyclohexyl)-2-((4-((S)-2- | 58% |
| | | | methoxy-1-((S)-2-oxo-4- | |
| | | | (trifluoromethyl)imidazolidin- | |
| | | | 1-yl)ethyl)pyridin-2-yl)amino)- | |
| | | | 2-oxoethyl)-1,2,5-oxadiazole- | |
| | | | 3-carboxamide-diastereomer 1 | |
| 286 | | diastereomer 1 | 3-ethyl-N-(1-(4-fluorocyclo- | 15 mg, |
| | | | hexyl)-2-((4-((S)-2-methoxy- | 60% |
| | | | 1-((S)-2-oxo-4-(trifluoro- | |
| | | | methyl)imidazolidin-1-yl)- | |
| | | | ethyl)pyridin-2-yl)amino)-2- | |
| | | | oxoethyl)isoxazole-4- | |
| | | | carboxamide-diastereomer 1 | |
| 287 | | diastereomer 1 | N-(1-(4-fluorocyclohexyl)-2- | 14 mg, |
| | | | ((4-((S)-2-methoxy-1-((S)-2- | 60% |
| | | | oxo-4-(trifluoromethyl) | |
| | | | imidazolidin-1- | |
| | | | yl)ethyl)pyridin-2-yl)amino)-2- | |
| | | | oxoethyl)-4-methyl-1,2,5- | |
| | | | oxadiazole-3-carboxamide- | |
| | | | diastereomer 1 | |
| 288 | | diastereomer 1 | N-(1-(4-fluorocyclohexyl)-2- | 14 mg, |
| | | | ((4-((S)-2-methoxy-1-((S)-2- | 60% |
| | | | oxo-4-(trifluoromethyl) | |
| | | | imidazolidin-1- | |
| | | | yl)ethyl)pyridin-2-yl)amino)-2- | |
| | | | oxoethyl)-1-methyl-1H- | |
| | | | pyrazole-5-carboxamide- | |
| | | | diastereomer 1 | |
| 289 | | diastereomer 1 | N-(1-(4-fluorocyclohexyl)-2- | 14 mg, |
| | | | ((4-((S)-2-methoxy-1-((S)-2- | 58% |
| | | | oxo-4-(trifluoromethyl) | |
| | | | imidazolidin-1- | |
| | | | yl)ethyl)pyridin-2-yl)amino)-2- | |
| | | | oxoethyl)-1-isopropyl-1H- | |
| | | | pyrazole-5-carboxamide- | |
| | | | diastereomer 1 | |

### Example 280

LC/MS: m/z=584.3 [M+H]⁺; rt: 1.81 min (LC/MS-method A). ¹H NMR (400 MHz, DMSO-d6) δ ppm: 10.64 (s, 1H), 8.48 (d, J=8.0 Hz, 1H), 8.30 (d, J=5.2 Hz, 1H), 8.01 (br s, 1H), 7.62 (d, J=2.3 Hz, 1H), 7.48 (d, J=2.0 Hz, 1H), 7.02-6.98 (m, 2H), 5.01 (dd, J=8.2, 5.1 Hz, 1H), 4.61-4.38 (m, 5H), 3.83 (dd, J=10.5, 8.2 Hz, 1H), 3.78-3.71 (m, 2H), 3.33-3.27 (m, 4H, overlapping water), 2.11-2.00 (m, 2H), 1.93-1.81 (m, 2H), 1.70-1.61 (m, 1H), 1.45-1.09 (m, 7H).

### Example 281

LC/MS: m/z=637.2 [M+H]⁺; rt: 1.88 min (LC/MS-method A). ¹H NMR (400 MHz, DMSO-d6) δ ppm: 10.73 (s, 1H), 9.06 (s, 1H), 8.57 (d, J=8.2 Hz, 1H), 8.30 (d, J=5.2 Hz, 1H), 8.01 (br s, 1H), 7.63 (d, J=2.3 Hz, 1H), 7.00 (dd, J=5.2, 1.5 Hz, 1H), 6.37 (tt, J=54.2, 3.5 Hz, 1H), 5.01 (dd, J=8.2, 5.1 Hz, 1H), 4.68-4.61 (m, 1H), 4.59-4.38 (m, 2H), 4.33 (td, J=14.6, 3.5 Hz, 2H), 3.83 (dd, J=10.5, 8.2 Hz, 1H), 3.78-3.70 (m, 2H), 3.33-3.27 (m, 4H, overlapping water), 2.09-1.98 (m, 2H), 1.90-1.79 (m, 2H), 1.72-1.63 (m, 1H), 1.44-1.09 (m, 4H).

### Example 282

LC/MS: m/z=656.2 [M+H]⁺; rt: 2.11 min (LC/MS-method A). ¹H NMR (400 MHz, DMSO-d6) δ ppm: 10.80 (s, 1H), 9.14 (d, J=8.3 Hz, 1H), 8.31 (d, J=5.2 Hz, 1H), 8.01 (br s, 1H), 7.63 (d, J=2.3 Hz, 1H), 7.01 (dd, J=5.2, 1.6 Hz, 1H), 5.21-5.09 (m, 2H), 5.01 (dd, J=8.1, 5.0 Hz, 1H), 4.72-4.66 (m, 1H), 4.58-4.36 (m, 2H), 3.83 (dd, J=10.5, 8.1 Hz, 1H), 3.79-3.70 (m, 2H), 3.33-3.26 (m, 4H, overlapping water), 2.10-1.98 (m, 2H), 1.90-1.78 (m, 2H), 1.72-1.63 (m, 1H), 1.47-1.09 (m, 4H).

### Example 283

LC/MS: m/z=571.2 [M+H]⁺; rt: 1.76 min (LC/MS-method A). ¹H NMR (400 MHz, DMSO-d6) δ ppm: 10.67 (s, 1H), 9.42 (s, 1H), 8.42 (d, J=8.0 Hz, 1H), 8.30 (d, J=5.2 Hz, 1H), 8.01 (br s, 1H), 7.62 (d, J=2.3 Hz, 1H), 7.00 (dd, J=5.2, 1.5 Hz, 1H), 5.01 (dd, J=8.1, 5.1 Hz, 1H), 4.62-4.38 (m, 3H), 3.83 (dd, J=10.5, 8.1 Hz, 1H), 3.78-3.70 (m, 2H), 3.33-3.26 (m, 4H, overlapping water), 2.36 (s, 3H), 2.11-2.01 (m, 2H), 1.93-1.74 (m, 2H), 1.70-1.61 (m, 1H), 1.46-1.10 (m, 4H).

### Example 284

LC/MS: m/z=571.3 [M+H]⁺; rt: 1.88 min (LC/MS-method A). ¹H NMR (400 MHz, DMSO-d6) δ ppm: 10.70 (s, 1H), 8.84 (q, J=1.1 Hz, 1H), 8.62 (d, J=8.2 Hz, 1H), 8.30 (d, J=5.2 Hz, 1H), 8.01 (br s, 1H), 7.63 (d, J=2.3 Hz, 1H), 7.00 (dd, J=5.2, 1.5 Hz, 1H), 5.01 (dd, J=8.2, 5.0 Hz, 1H), 4.64-4.39 (m, 3H), 3.83 (dd, J=10.5, 8.2 Hz, 1H), 3.79-3.71 (m, 2H), 3.33-3.28 (m, 4H, overlapping water), 2.12-1.98 (m, 5H), 1.94-1.80 (m, 2H), 1.71-1.63 (m, 1H), 1.45-1.09 (m, 4H).

### Example 285

LC/MS: m/z=584.2 [M+H]⁺; rt: 2.09 min (LC/MS-method A). ¹H NMR (400 MHz, DMSO-d6) δ ppm: 10.75 (s, 1H), 9.17 (d, J=8.0 Hz, 1H), 8.30 (d, J=5.2 Hz, 1H), 8.01 (br s, 1H), 7.63 (d, J=2.4 Hz, 1H), 7.01 (dd, J=5.2, 1.5 Hz, 1H), 5.01 (dd, J=8.2, 5.0 Hz, 1H), 4.67-4.61 (m, 1H), 4.60-4.38 (m, 2H), 3.83 (dd, J=10.5, 8.2 Hz, 1H), 3.79-3.71 (m, 2H), 3.33-3.28 (m, 4H, overlapping water), 2.89 (q, J=7.5 Hz, 2H), 2.10-1.99 (m, 2H), 1.94-1.80 (m, 2H), 1.72-1.63 (m, 1H), 1.45-1.11 (m, 7H).

### Example 286

LC/MS: m/z=585.3 [M+H]⁺; rt: 1.88 min (LC/MS-method A). ¹H NMR (400 MHz, DMSO-d6) δ ppm: 10.66 (s, 1H), 9.40 (s, 1H), 8.44 (d, J=8.0 Hz, 1H), 8.30 (d, J=5.1 Hz, 1H), 8.01 (br s, 1H), 7.62 (d, J=2.3 Hz, 1H), 7.00 (dd, J=5.2, 1.5 Hz, 1H), 5.01 (dd, J=8.2, 5.1 Hz, 1H), 4.62-4.38 (m, 3H), 3.83 (dd, J=10.5, 8.2 Hz, 1H), 3.78-3.70 (m, 2H), 3.33-3.26 (m, 4H, overlapping water), 2.82 (q, J=7.5 Hz, 2H), 2.10-2.01 (m, 2H), 1.93-1.74 (m, 2H), 1.71-1.61 (m, 1H), 1.45-1.12 (m, 6H).

### Example 287

LC/MS: m/z=572.3 [M+H]⁺; rt: 1.94 min (LC/MS-method A). ¹H NMR (400 MHz, DMSO-d6) δ ppm: 10.75 (s, 1H), 9.13 (d, J=8.0 Hz, 1H), 8.30 (d, J=5.2 Hz, 1H), 8.01 (br s, 1H), 7.63 (d, J=2.3 Hz, 1H), 7.01 (dd, J=5.2, 1.6 Hz, 1H), 5.01 (dd, J=8.2, 5.0 Hz, 1H), 4.67-4.38 (m, 3H), 3.83 (dd, J=10.6, 8.2 Hz, 1H), 3.79-3.71 (m, 2H), 3.33-3.26 (m, 4H, overlapping water), 2.47 (s, 3H), 2.11-1.99 (m, 2H), 1.94-1.81 (m, 2H), 1.72-1.63 (m, 1H), 1.46-1.10 (m, 4H).

### Example 288

LC/MS: m/z=570.2 [M+H]⁺; rt: 1.70 min (LC/MS-method A). ¹H NMR (400 MHz, DMSO-d6) δ ppm: 10.65 (s, 1H), 8.48 (d, J=8.0 Hz, 1H), 8.30 (d, J=5.2 Hz, 1H), 8.01 (br s, 1H), 7.62 (d, J=2.3 Hz, 1H), 7.47 (d, J=2.1 Hz, 1H), 7.03 (d, J=2.1 Hz, 1H), 7.00 (dd, J=5.2, 1.5 Hz, 1H), 5.00 (dd, J=8.2, 5.1 Hz, 1H), 4.61-4.38 (m, 3H), 4.01 (s, 3H), 3.83 (dd, J=10.5, 8.2 Hz, 1H), 3.78-3.71 (m, 2H), 3.33-3.26 (m, 4H, overlapping water), 2.11-2.00 (m, 2H), 1.93-1.81 (m, 2H), 1.70-1.61 (m, 1H), 1.46-1.08 (m, 4H).

### Example 289

LC/MS: m/z=598.3 [M+H]⁺; rt: 1.93 min (LC/MS-method A). ¹H NMR (400 MHz, DMSO-d6) δ ppm: 10.63 (s, 1H), 8.47 (d, J=7.9 Hz, 1H), 8.30 (d, J=5.2 Hz, 1H), 8.01 (br s, 1H), 7.62 (d, J=2.3 Hz, 1H), 7.50 (d, J=2.0 Hz, 1H), 7.00 (dd, J=5.2, 1.5 Hz, 1H), 6.93 (d, J=2.0 Hz, 1H), 5.36 (hept, J=6.6 Hz, 1H), 5.01 (dd, J=8.2, 5.0 Hz, 1H), 4.61-4.38 (m, 3H), 3.83 (dd, J=10.5, 8.2 Hz, 1H), 3.78-3.71 (m, 2H), 3.33-3.27 (m, 4H, overlapping water), 2.11-2.00 (m, 2H), 1.94-1.80 (m, 2H), 1.71-1.60 (m, 1H), 1.46-1.09 (m, 10H).

### Example 290: 4-Ethyl-N—((S)-1-((1r,4S)-4-fluorocyclohexyl)-2-oxo-2-((4-(((S)-2-oxo-4-(trifluoromethyl)imidazolidin-1-yl)methyl)pyridin-2-yl)amino)ethyl)-1,2,5-oxadiazole-3-carboxamide

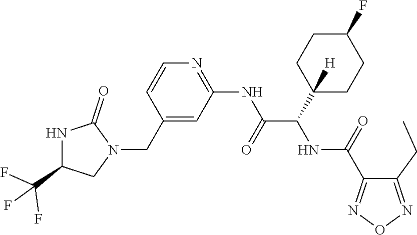

To a mixture of (S)-2-amino-2-((1r,4S)-4-fluorocyclohexyl)-N-(4-(((S)-2-oxo-4-(trifluoromethyl)imidazolidin-1-yl)methyl)pyridin-2-yl)acetamide (Intermediate 66, 20 mg), 4-ethyl-1,2,5-oxadiazole-3-carboxylic acid (8 mg) and DCM (1.0 ml) DIPEA (40 μl) and T3P (90 μl) were added. After 1 h the mixture was concentrated in vacuo and the residue was purified by preparative RP HPLC (Agilent Prep C18, 21.2 mm×250 mm, 10 μm; 50 ml/min, from 90% H₂O/10% ACN to 10% H₂O/90% ACN in 12.5 min). The pure product containing fractions were combined, the ACN was partly removed and the residue freeze dried to yield 17 mg of the title compound. LC/MS: m/z=542.1 [M+H]⁺; rt: 2.02 mi (LC/MS-method A). ¹H NMR (400 MHz, DMSO-d6) δ ppm: 10.75 (s, 1H), 9.17 (d, J=8.0 Hz, 1H), 8.30 (d, J=5.1 Hz, 1H), 7.97 (br s, 1H), 7.65 (d, J=2.3 Hz, 1H), 6.96 (dd, J=5.1, 1.5 Hz, 1H), 4.68-14.61 (m, 1H), 4.60-4.35 (m, 3H), 4.22 (d, J=16.2 Hz, 1H), 3.62-3.55 (m, 1H), 3.35-3.28 (m, 1H, overlapping water), 2.89 (q, J=7.5 Hz, 2H), 2.11-1.99 (m, 2H), 1.94-1.80 (m, 2H), 1.71-1.62 (m, 1H), 1.45-41.11 (m, 7H).

Following the procedure described in Example 290 with reaction times ranging from 0.20 min to 2 h the following examples were prepared:

| Ex. | Acid | Structure | Name | Yield |
|---|---|---|---|---|
| 291 | | | N-((S)-1-((1r,4S)-4- | 16 mg, |
| | | | fluorocyclohexyl)-2-oxo-2-((4- | 65% |
| | | | (((S)-2-oxo-4- | |
| | | | (trifluoromethyl)imidazolidin- | |
| | | | 1-yl)methyl)pyridin-2- | |
| | | | yl)amino)ethyl)-4-methyl- | |
| | | | 1,2,5-oxadiazole-3- | |
| | | | carboxamide | |
| 292 | | | 4-cyclopropyl-N-((S)-1- | 17 mg, |
| | | | ((1r,4S)-4-fluorocyclohexyl)-2- | 70% |
| | | | oxo-2-((4-(((S)-2-oxo-4- | |
| | | | (trifluoromethyl)imidazolidin- | |
| | | | 1-yl)methyl)pyridin-2- | |
| | | | yl)amino)ethyl)-1,2,5- | |
| | | | oxadiazole-3-carboxamide | |
| 293 | | | 1-ethyl-N-((S)-1-((1r,4S)-4- | 16 mg, |
| | | | fluorocyclohexyl)-2-oxo-2-((4- | 62% |
| | | | (((S)-2-oxo-4- | |
| | | | (trifluoromethyl)imidazolidin- | |
| | | | 1-yl)methyl)pyridin-2- | |
| | | | yl)amino)ethyl)-1H-pyrazole- | |
| | | | 5-carboxamide | |
| 294 | | | N-((S)-1-((1r,4S)-4- | 16 mg, |
| | | | fluorocyclohexyl)-2-oxo-2-((4- | 64% |
| | | | (((S)-2-oxo-4- | |
| | | | (trifluoromethyl)imidazolidin- | |
| | | | 1-yl)methyl)pyridin-2- | |
| | | | yl)amino)ethyl)-1-methyl-1H- | |
| | | | pyrazole-5-carboxamide | |

### Example 291

LC/MS: m/z=528.1 [M+H]⁺; rt: 1.87 min (LC/MS-method A). ¹H NMR (400 MHz, DMSO-d6) δ ppm: 10.75 (s, 1H), 9.13 (d, J=8.1 Hz, 1H), 8.30 (d, J=5.0 Hz, 1H), 7.98 (br s, 1H), 7.65 (d, J=2.3 Hz, 1H), 6.96 (dd, J=5.0, 1.5 Hz, 1H), 4.67-4.60 (m, 1H), 4.60-4.35 (m, 3H), 4.22 (d, J=16.2 Hz, 1H), 3.62-3.55 (m, 1H), 3.34-3.28 (m, 1H, overlapping water), 2.47 (s, 3H), 2.11-1.99 (m, 2H), 1.95-1.81 (m, 2H), 1.72-1.62 (m, 1H), 1.45-1.10 (m, 4H).

### Example 292

LC/MS: m/z=554.2 [M+H]⁺; rt: 2.07 min (LC/MS-method A). ¹H NMR (400 MHz, DMSO-d6) δ ppm: 10.76 (s, 1H), 9.21 (d, J=7.9 Hz, 1H), 8.30 (d, J=5.2 Hz, 1H), 7.98 (br s, 1H), 7.65 (d, J=2.3 Hz, 1H), 6.96 (dd, J=5.2, 1.5 Hz, 1H), 4.66 (m, 1H), 4.61-4.35 (m, 3H), 4.22 (d, J=16.2 Hz, 1H), 3.63-3.55 (m, 1H), 3.34-3.29 (m, 1H, overlapping water), 2.27 (tt, J=8.4, 5.0 Hz, 1H), 2.11-1.99 (m, 2H), 1.94-1.81 (m, 2H), 1.72-1.63 (m, 1H), 1.52-1.08 (m, 6H), 1.01-0.94 (m, 2H).

### Example 293

LC/MS: m/z=540.2 [M+H]⁺; rt: 1.74 min (LC/MS-method A). ¹H NMR (400 MHz, DMSO-d6) δ ppm: 10.64 (s, 1H), 8.48 (d, J=8.0 Hz, 1H), 8.29 (d, J=5.1 Hz, 1H), 7.98 (br s, 1H), 7.64 (d, J=2.3 Hz, 1H), 7.48 (d, J=2.1 Hz, 1H), 7.00 (d, J=2.1 Hz, 1H), 6.95 (dd, J=5.1, 1.5 Hz, 1H), 4.62-4.35 (m, 6H), 4.21 (d, J=16.2 Hz, 1H), 3.62-3.54 (m, 1H), 3.36-3.28 (m, 1H, overlapping water), 2.11-2.00 (m, 2H), 1.93-1.81 (m, 2H), 1.65 (d, J=11.2 Hz, 1H), 1.45-1.09 (m, 7H).

### Example 294

LC/MS: m/z=526.1 [M+H]⁺; rt: 1.63 min (LC/MS-method A). ¹H NMR (400 MHz, DMSO-d6) δ ppm: 10.65 (s, 1H), 8.47 (d, J=8.0 Hz, 1H), 8.29 (d, J=5.1 Hz, 1H), 7.98 (br s, 1H), 7.64 (d, J=2.3 Hz, 1H), 7.47 (d, J=2.1 Hz, 1H), 7.03 (d, J=2.1 Hz, 1H), 6.95 (dd, J=5.1, 1.5 Hz, 1H), 4.62-4.35 (m, 4H), 4.21 (d, J=16.2 Hz, 1H), 4.01 (s, 3H), 3.62-3.54 (m, 1H), 3.34-3.28 (m, 1H, overlapping water), 2.11-2.00 (m, 2H), 1.94-1.80 (m, 2H), 1.70-1.60 (m, 1H), 1.46-1.08 (m, 4H).

### Example 295: 4-Cyclopropyl-N-(1-(4-fluoro-4-methylcyclohexyl)-2-((4-((S)-2-methoxy-1-((S)-2-oxo-4-(trifluoromethyl)imidazolidin-1-yl)ethyl)pyridin-2-yl)amino)-2-oxoethyl)-1,2,5-oxadiazole-3-carboxamide—Diastereomer 1

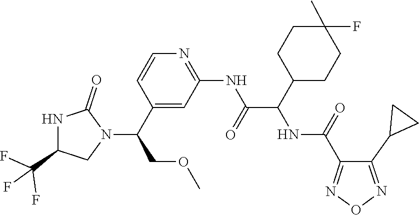 [SMILES: COC[C@H](c1ccnc(NC(=O)C(NC(=O)c2nonc2C2CC2)C2CCC(C)(F)CC2)c1)N1C[C@@H](C(F)(F)F)NC1=O]

To a mixture of 2-amino-2-(4-fluoro-4-methylcyclohexyl)-N-(4-((S)-2-methoxy-1-((S)-2-oxo-4-(trifluoromethyl)imidazolidin-1-yl)ethyl)pyridin-2-yl)acetamide hydrochloride (Intermediate 69-DS1; 20 mg), 4-cyclopropyl-1,2,5-oxadiazole-3-carboxylic acid (7 mg) and DCM (1.0 ml), DIPEA (34 μl) and T3P (70 μl) were added. After 5 h the mixture was concentrated in vacuo and the residue was purified by preparative RP HPLC (Agilent Prep C18, 21.2 mm×250 mm, 10 μm; 50 ml/min, from 90% H₂O/10% ACN to 10% H₂O/90% ACN in 12.5 min). The pure product containing fractions were combined, the ACN was partly removed and the residue freeze dried to yield 12 mg of the title compound. LC/MS: m/z=612.2 [M+H]⁺; rt: 2.23 min (LC/MS-method A). ¹H NMR (400 MHz, DMSO-d6) δ ppm: 10.77 (s, 1H), 9.23 (d, J=8.0 Hz, 1H), 8.31 (d, J=5.3 Hz, 1H), 8.02 (br s, 1H), 7.63 (d, J=2.3 Hz, 1H), 7.01 (dd, J=5.3, 1.5 Hz, 1H), 5.01 (dd, J=8.2, 5.0 Hz, 1H), 4.70-4.63 (m, 1H), 4.50-4.38 (m, 1H), 3.83 (dd, J=10.6, 8.2 Hz, 1H), 3.79-3.71 (m, 2H), 3.36-3.27 (m, 4H), 2.27 (tt, J=8.3, 5.0 Hz, 1H), 1.93-1.76 (m, 3H), 1.72-1.64 (m, 1H), 1.59-1.21 (m, 8H), 1.16-1.09 (m, 2H), 1.01-0.94 (m, 2H).

The following diastereomers were synthesised as described in example 295 with a reaction time of 40 min:

| Ex. | Amine | Structure | Name | Yield |
|---|---|---|---|---|
| 296 | Int. 69- | diastereomer 2 | 4-cyclopropyl-N-(1-(4-fluoro-4- | 1 mg, |
| | DS2 | | methylcyclohexyl)-2-((4-((S)-2- | 31% |
| | | | methoxy-1-((S)-2-oxo-4- | |
| | | | (trifluoromethyl) imidazolidin-1- | |
| | | | yl)ethyl)pyridin-2-yl)amino)-2- | |
| | | | oxoethyl)-1,2,5-oxadiazole-3- | |
| | | | carboxamide-diastereomer 2 | |
| 297 | Int. 69- | diastereomer 4 | 4-cyclopropyl-N-(1-(4-fluoro-4- | 10 mg, |
| | DS4 | | methylcyclohexyl)-2-((4-((S)-2- | 60% |
| | | | methoxy-1-((S)-2-oxo-4- | |
| | | | (trifluoromethyl) imidazolidin-1- | |
| | | | yl)ethyl)pyridin-2-yl)amino)-2- | |
| | | | oxoethyl)-1,2,5-oxadiazole-3- | |
| | | | carboxamide-diastereomer 4 | |

### Example 296

LC/MS: m/z=612.2 [M+H]⁺; rt: 2.22 min (LC/MS-method A). ¹H NMR (400 MHz, DMSO-d6) δ ppm: 10.72 (s, 1H), 9.27 (d, J=8.2 Hz, 1H), 8.30 (d, J=5.2 Hz, 1H), 8.02 (br s, 1H), 7.63 (d, J=2.4 Hz, 1H), 7.03 (br d, J=5.2 Hz, 1H), 5.01 (dd, J=8.2, 5.1 Hz, 1H), 4.78-4.71 (m, 1H), 4.50-4.39 (m, 1H), 3.86-3.71 (m, 3H), 3.37-3.29 (m, 4H, overlapping water), 2.27 (tt, J=8.4, 5.0 Hz, 1H), 2.07-1.50 (m, 7H), 1.43-1.27 (m, 5H), 1.16-1.09 (m, 2H), 1.00-0.94 (m, 2H).

### Example 297

LC/MS: m/z=612.2 [M+H]⁺; rt: 2.23 min (LC/MS-method A). ¹H NMR (400 MHz, DMSO-d6) δ ppm: 10.75 (s, 1H), 9.23 (d, J=8.1 Hz, 1H), 8.30 (d, J=5.2 Hz, 1H), 8.02 (br s, 1H), 7.62 (d, J=2.3 Hz, 1H), 7.02 (dd, J=5.2, 1.5 Hz, 1H), 5.01 (dd, J=8.2, 5.0 Hz, 1H), 4.69-4.63 (m, 1H), 4.49-4.39 (m, 1H), 3.83 (dd, J=10.6, 8.2 Hz, 1H), 3.79-3.71 (m, 2H), 3.36-3.29 (m, 4H, overlapping water), 2.27 (tt, J=8.4, 5.0 Hz, 1H), 1.93-1.77 (m, 3H), 1.72-1.64 (m, 1H), 1.59-1.23 (m, 8H), 1.16-1.09 (m, 2H), 1.00-0.95 (m, 2H).

Following the procedure described in Example 295 using Intermediate 69-DS1 with a reaction time of 45 min the following examples were prepared:

| Ex. | Acid | Structure | Name | Yield |
|---|---|---|---|---|
| 298 | | | N-(1-(4-fluoro-4-methylcyclohexyl)- | 6 mg, |
| | | | 2-((4-((S)-2-methoxy-1-((S)-2-oxo- | 49% |
| | | | 4-(trifluoromethyl)imidazolidin-1- | |
| | | | yl)ethyl)pyridin-2-yl)amino)-2- | |
| | | | oxoethyl)-4-methyl-1,2,5- | |
| | | | oxadiazole-3-carboxamide- | |
| | | | diastereomer 1 | |
| 299 | | | 1-ethyl-N-(1-(4-fluoro-4- | 6 mg, |
| | | | methylcyclohexyl)-2-((4-((S)-2- | 46% |
| | | | methoxy-1-((S)-2-oxo-4- | |
| | | | (trifluoromethyl)imidazolidin-1- | |
| | | | yl)ethyl)pyridin-2-yl)amino)-2- | |
| | | | oxoethyl)-1H-pyrazole-5- | |
| | | | carboxamide-diastereomer 1 | |

### Example 298

LC/MS: m/z=586.2 [M+H]⁺; rt: 2.07 min (LC/MS-method A). ¹H NMR (400 MHz, DMSO-d6) δ ppm: 10.75 (s, 1H), 9.15 (d, J=8.1 Hz, 1H), 8.30 (d, J=5.2 Hz, 1H), 8.02 (br s, 1H), 7.63 (d, J=2.3 Hz, 1H), 7.01 (dd, J=5.2, 1.5 Hz, 1H), 5.01 (dd, J=8.2, 5.0 Hz, 1H), 4.67-4.61 (m, 1H), 4.49-4.39 (m, 1H), 3.83 (dd, J=10.5, 8.2 Hz, 1H), 3.79-3.71 (m, 2H), 3.34-3.27 (m, 4H, overlapping water), 2.47 (s, 3H), 1.94-1.76 (m, 3H), 1.72-1.64 (m, 1H), 1.55-1.22 (m, 8H).

### Example 299

LC/MS: m/z=598.2 [M+H]⁺; rt: 1.94 min (LC/MS-method A). ¹H NMR (400 MHz, DMSO-d6) δ ppm: 10.65 (s, 1H), 8.50 (d, J=8.0 Hz, 1H), 8.30 (d, J=5.2 Hz, 1H), 8.02 (br s, 1H), 7.62 (d, J=2.3 Hz, 1H), 7.48 (d, J=2.0 Hz, 1H), 7.01-6.98 (m, 2H), 5.01 (dd, J=8.2, 5.0 Hz, 1H), 4.58-4.52 (m, 1H), 4.49-4.40 (m, 3H), 3.83 (dd, J=10.5, 8.2 Hz, 1H), 3.78-3.70 (m, 2H), 3.32-3.26 (m, 4H, overlapping water), 1.91-1.76 (m, 3H), 1.74-1.67 (m, 1H), 1.56-1.22 (m, 11H).

### Example 300: 3-ethyl-N-((1S)-2-((4-((S)-2-methoxy-1-((S)-2-oxo-4-(trifluoromethyl)-imidazolidin-1-yl)ethyl)pyridin-2-yl)amino)-2-oxo-1-(4-(trifluoromethyl)-cyclohexyl)ethyl)isoxazole-4-carboxamide

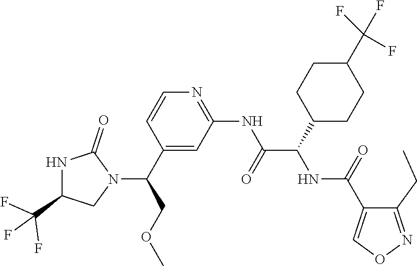 [SMILES: CCc1nocc1C(=O)N[C@H](C(=O)Nc1cc([C@@H](COC)N2C[C@@H](C(F)(F)F)NC2=O)ccn1)C1CCC(C(F)(F)F)CC1]

Following the procedure described for Example 33 using 30 mg (2S)-2-amino-N-(4-((S)-2-methoxy-1-((S)-2-oxo-4-(trifluoromethyl)imidazolidin-1-yl)ethyl)pyridin-2-yl)-2-(4-(trifluoromethyl)cyclohexyl)acetamide (Intermediate 73), 29 mg of the title compound was obtained. LC/MS: m/z=635.2 [M+H]⁺; rt: 2.18 min (LC/MS-method A). ¹H NMR (400 MHz, DMSO-d6) δ ppm: 10.75 (s, 1H), 9.42 (s, 1H), 8.46 (d, J=8.0 Hz, 1H), 8.31 (d, J=5.2 Hz, 1H), 7.99 (br s, 1H), 7.63 (d, J=2.4 Hz, 1H), 7.02 (dd, J=5.2, 1.6 Hz, 1H), 5.02 (dd, J=8.0, 5.1 Hz, 1H), 4.62-4.55 (m, 1H), 4.50-4.39 (m, 1H), 3.83 (dd, J=10.5, 8.1 Hz, 1H), 3.79-3.71 (m, 2H), 3.33-3.26 (m, 4H), 2.83 (q, J=7.5 Hz, 2H), 2.29-2.14 (m, 1H), 1.99-1.67 (m, 5H), 1.36-1.10 (m, 7H).

Examples 301-303 were prepared analogously to Example 300.

| Ex. | Acid | Structure | Name | Yield |
|---|---|---|---|---|
| 301 | | | 1-ethyl-N-((1S)-2-((4-((S)-2- | 26 mg, |
| | | | methoxy-1-((S)-2-oxo-4- | 70% |
| | | | (trifluoromethyl)imidazolidin-1- | |
| | | | yl)ethyl)pyridin-2-yl)amino)-2- | |
| | | | oxo-1-(4-(trifluoromethyl)- | |
| | | | cyclohexyl)ethyl)-1H-pyrazole-5- | |
| | | | carboxamide | |
| 302 | | | N-((1S)-2-((4-((S)-2-methoxy-1- | 26 mg, |
| | | | ((S)-2-oxo-4- | 71% |
| | | | (trifluoromethyl)imidazolidin-1- | |
| | | | yl)ethyl)pyridin-2-yl)amino)-2- | |
| | | | oxo-1-(4-(trifluoromethyl)- | |
| | | | cyclohexyl)ethyl)-1-methyl-1H- | |
| | | | pyrazole-5-carboxamide | |
| 303 | | | 1-isopropyl-N-((1S)-2-((4-((S)-2- | 28 mg, |
| | | | methoxy-1-((S)-2-oxo-4- | 73% |
| | | | (trifluoromethyl)imidazolidin-1- | |
| | | | yl)ethyl)pyridin-2-yl)amino)-2- | |
| | | | oxo-1-(4- | |
| | | | (trifluoromethyl)cyclohexyl)ethyl)- | |
| | | | 1H-pyrazole-5-carboxamide | |

### Example 301

LC/MS: m/z=634.3 [M+H]⁺; rt: 2.12 min (LC/MS-method A). ¹H NMR (400 MHz, DMSO-d6) δ ppm: 10.76 (s, 1H), 8.49 (d, J=8.0 Hz, 1H), 8.31 (d, J=5.3 Hz, 1H), 7.99 (br s, 1H), 7.64 (d, J=2.4 Hz, 1H), 7.49 (d, J=2.0 Hz, 1H), 7.03 (dd, J=5.3, 1.6 Hz, 1H), 7.01 (d, J=2.0 Hz, 1H), 5.02 (dd, J=8.1, 5.0 Hz, 1H), 4.58-4.40 (m, 4H), 3.83 (dd, J=10.5, 8.1 Hz, 1H), 3.78-3.71 (m, 2H), 3.33-3.27 (m, 4H), 2.30-2.15 (m, 1H), 1.98-1.82 (m, 4H), 1.75-1.67 (m, 1H), 1.37-1.09 (m, 7H).

### Example 302

LC/MS: m/z=620.2 [M+H]⁺; rt: 2.03 min (LC/MS-method A). ¹H NMR (400 MHz, DMSO-d6) δ ppm: 10.79 (s, 1H), 8.49 (d, J=8.0 Hz, 1H), 8.31 (d, J=5.2 Hz, 1H), 7.99 (br s, 1H), 7.64 (d, J=2.4 Hz, 1H), 7.47 (d, J=2.0 Hz, 1H), 7.06-7.01 (m, 2H), 5.01 (dd, J=8.1, 5.1 Hz, 1H), 4.58-4.52 (m, 1H), 4.49-4.39 (m, 1H), 4.02 (s, 3H), 3.83 (dd, J=10.5, 8.1 Hz, 1H), 3.79-3.71 (m, 2H), 3.34-3.26 (m, 4H), 2.30-2.15 (m, 1H), 1.97-1.82 (m, 4H), 1.76-1.67 (m, 1H), 1.37-1.08 (in, 4H).

### Example 303

LC/MS: m/z=646.2 [M+H]⁺; rt: 2.23 min (LC/MS-method A). ¹H NMR (400 MHz, DMSO-d6) δ ppm: 10.76 (s, 1H), 8.47 (d, J=7.9 Hz, 1H), 8.31 (d, J=5.3 Hz, 1H), 7.99 (br s, 1H), 7.64 (d, J=2.4 Hz, 1H), 7.51 (d, J=2.0 Hz, 1H), 7.03 (dd, J=5.3, 1.6 Hz, 1H), 6.95 (d, J=2.0 Hz, 1H), 5.37 (hept, J=6.5 Hz, 1H), 5.02 (dd, J=8.1, 5.1 Hz, 1H), 4.57-4.50 (m, 1H), 4.49-4.39 (m, 1H), 3.83 (dd, J=10.5, 8.1 Hz, 1H), 3.79-3.71 (m, 2H), 3.34-3.27 (m, 4H), 2.30-2.15 (m, 1H), 1.98-1.81 (m, 4H), 1.75-1.67 (m, 1H), 1.41-1.08 (m, 10H).

### Example 304: N—((S)-1-((S)-3,3-difluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-4-ethylisoxazole-3-carboxamide diastereomer 2

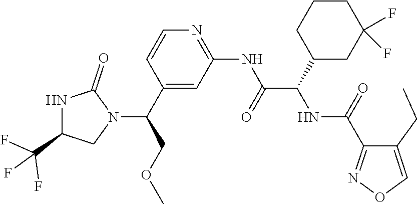 [SMILES: CCc1conc1C(=O)N[C@H](C(=O)Nc1cc([C@@H](COC)N2C[C@@H](C(F)(F)F)NC2=O)ccn1)C1CCCC(F)(F)C1]

Following the procedure described for Example 39 using 30 mg (S)-2-amino-2-((S)-3,3-difluorocyclohexyl)-N-(4-((S)-2-methoxy-1-((S)-2-oxo-4-(trifluoromethyl)-imidazolidin-1-yl)ethyl)pyridin-2-yl)acetamide (Intermediate 74-DS2) 27 mg of the title compound was obtained. LC/MS: m/z=603.3 [M+H]⁺; rt: 2.10 min (LC/MS-method A). ¹H NMR (400 MHz, DMSO-d6) δ ppm: 10.79 (s, 1H), 8.87 (t, J=0.9 Hz, 1H), 8.80 (d, J=8.3 Hz, 1H), 8.31 (d, J=5.3 Hz, 1H), 7.99 (br s, 1H), 7.64 (d, J=2.3 Hz, 1H), 7.03 (dd, J=5.3, 1.6 Hz, 1H), 5.03 (dd, J=8.0, 5.1 Hz, 1H), 4.78-4.72 (m, 1H), 4.50-4.39 (m, 1H), 3.84 (dd, J=10.5, 8.1 Hz, 1H), 3.79-3.71 (m, 2H), 3.33-3.26 (m, 4H), 2.59-2.51 (m, 2H), 2.26-2.15 (m, 1H), 2.10-1.92 (m, 2H), 1.85-1.59 (m, 4H), 1.46-1.19 (m, 2H), 1.13 (t, J=7.5 Hz, 3H).

Examples 305-307 were prepared analogously to Example 304 using Intermediate 74-DS1 or Intermediate 74-DS2. Examples 308-309 were prepared analogously to Example 304 using the procedure described for Example 33 using Intermediate 74-DS1.

| Ex. | Acid | Structure | Name | Yield |
|---|---|---|---|---|
| 305 | | | N-((S)-1-((S)-3,3-difluoro- | 24 mg, |
| | | | cyclohexyl)-2-((4-((S)-2- | 68% |
| | | | methoxy-1-((S)-2-oxo-4- | |
| | | | (trifluoromethyl)imidazolidin- | |
| | | | 1-yl)ethyl)pyridin-2-yl)amino)- | |
| | | | 2-oxoethyl)-4-ethylisoxazole- | |
| | | | 3-carboxamide (diastereomer 1) | |
| 306 | | | N-((S)-1-((S)-3,3- | 27 mg, |
| | | | difluorocyclohexyl)-2-((4-((S)- | 70% |
| | | | 2-methoxy-1-((S)-2-oxo-4- | |
| | | | (trifluoromethyl)imidazolidin- | |
| | | | 1-yl)ethyl)pyridin-2-yl)amino)- | |
| | | | 2-oxoethyl)-3-ethylisoxazole- | |
| | | | 4-carboxamide (diastereomer 2) | |
| 307 | | | N-((S)-1-((S)-3,3- | 21 mg, |
| | | | difluorocyclohexyl)-2-((4-((S)- | 59% |
| | | | 2-methoxy-1-((S)-2-oxo-4- | |
| | | | (trifluoromethyl)imidazolidin- | |
| | | | 1-yl)ethyl)pyridin-2-yl)amino)- | |
| | | | 2-oxoethyl)-3-ethylisoxazole- | |
| | | | 4-carboxamide (diastereomer 1) | |
| 308 | | | N-((S)-1-((S)-3,3- | 40 mg, |
| | | | difluorocyclohexyl)-2-((4-((S)- | 96% |
| | | | 2-methoxy-1-((S)-2-oxo-4- | |
| | | | (trifluoromethyl)imidazolidin- | |
| | | | 1-yl)ethyl)pyridin-2-yl)amino)- | |
| | | | 2-oxoethyl)-1-ethyl-1H- | |
| | | | pyrazole-5-carboxamide | |
| | | | (diastereomer 1) | |
| 309 | | | N-((S)-1-((S)-3,3- | 35 mg, |
| | | | difluorocyclohexyl)-2-((4-((S)- | 90% |
| | | | 2-methoxy-1-((S)-2-oxo-4- | |
| | | | (trifluoromethyl)imidazolidin- | |
| | | | 1-yl)ethyl)pyridin-2-yl)amino)- | |
| | | | 2-oxoethyl)-1-isopropyl-1H- | |
| | | | pyrazole-5-carboxamide | |
| | | | (diastereomer 2) | |

### Example 305

LC/MS: m/z=603.3 [M+H]⁺; rt: 2.12 min (LC/MS-method A). ¹H NMR (400 MHz, DMSO-d6) δ ppm: 10.80 (s, 1H), 8.87 (t, J=0.9 Hz, 1H), 8.72 (d, J=8.1 Hz, 1H), 8.32 (d, J=5.2 Hz, 1H), 7.99 (br s, 1H), 7.64 (d, J=2.3 Hz, 1H), 7.04 (dd, J=5.2, 1.6 Hz, 1H), 5.03 (dd, J=8.1, 5.1 Hz, 1H), 4.73-4.66 (m, 1H), 4.49-4.39 (m, 1H), 3.84 (dd, J=10.5, 8.1 Hz, 1H), 3.79-3.71 (m, 2H), 3.33-3.27 (m, 4H), 2.59-2.52 (m, 2H), 2.26-2.15 (m, 1H), 2.04-1.91 (m, 2H), 1.85-1.59 (m, 4H), 1.44-1.30 (m, 1H), 1.21-1.07 (m, 4H).

### Example 306

LC/MS: m/z=603.3 [M+H]⁺; rt: 1.98 min (LC/MS-method A). ¹H NMR (400 MHz, DMSO-d6) δ ppm: 10.78 (s, 1H), 9.45 (s, 1H), 8.50 (d, J=8.2 Hz, 1H), 8.31 (d, J=5.3 Hz, 1H), 7.99 (br s, 1H), 7.63 (d, J=2.3 Hz, 1H), 7.03 (dd, J=5.3, 1.6 Hz, 1H), 5.03 (dd, J=8.0, 5.1 Hz, 1H), 4.78-4.72 (m, 1H), 4.49-4.39 (m, 1H), 3.83 (dd, J=10.5, 8.0 Hz, 1H), 3.79-3.70 (m, 2H), 3.33-3.24 (m, 4H), 2.83 (q, J=7.5 Hz, 2H), 2.21-1.93 (m, 3H), 1.86-1.58 (m, 4H), 1.46-1.32 (m, 1H), 1.31-1.12 (m, 4H)

### Example 307

LC/MS: m/z=603.2 [M+H]⁺; rt: 1.98 min (LC/MS-method A). ¹H NMR (400 MHz, DMSO-d6) δ ppm: 10.81 (s, 1H), 9.44 (s, 1H), 8.52 (d, J=8.0 Hz, 1H), 8.32 (d, J=5.3 Hz, 1H), 7.99 (br s, 1H), 7.64 (d, J=2.3 Hz, 1H), 7.04 (dd, J=5.3, 1.6 Hz, 1H), 5.03 (dd, J=8.0, 5.1 Hz, 1H), 4.72-4.66 (m, 1H), 4.49-4.38 (m, 1H), 3.84 (dd, J=10.5, 8.0 Hz, 1H), 3.80-3.70 (m, 2H), 3.31 (s, 3H), 3.28 (dd, J=10.1, 3.8 Hz, 1H), 2.83 (q, J=7.5 Hz, 2H), 2.20-2.07 (m, 1H), 1.98 (d, J=15.0 Hz, 2H), 1.86-1.59 (m, 4H), 1.45-1.31 (m, 1H), 1.22-1.08 (m, 4H)

### Example 308

LC/MS: m/z=602.3 [M+H]⁺; rt: 1.91 min (LC/MS-method A). ¹H NMR (400 MHz, DMSO-d6) δ ppm: 10.81 (s, 1H), 8.56 (d, J=7.9 Hz, 1H), 8.32 (d, J=5.3 Hz, 1H), 7.99 (br s, 1H), 7.64 (d, J=2.3 Hz, 1H), 7.49 (d, J=2.0 Hz, 1H), 7.05 (dd, J=5.3, 1.6 Hz, 1H), 7.02 (d, J=2.0 Hz, 1H), 5.03 (dd, J=8.0, 5.1 Hz, 1H), 4.68-4.61 (m, 1H), 4.50-4.38 (m, 3H), 3.84 (dd, J=10.5, 8.0 Hz, 1H), 3.80-3.72 (m, 2H, partially overlapping water), 3.32-3.26 (m, 4H), 2.26-2.14 (m, 1H), 2.06-1.89 (m, 2H), 1.87-1.61 (m, 4H), 1.46-1.32 (m, 1H), 1.27 (t, J=7.1 Hz, 3H), 1.21-1.07 (m, 1H)

### Example 309

LC/MS: m/z=616.3 [M+H]⁺; rt: 2.03 min (LC/MS-method A). ¹H NMR (400 MHz, DMSO-d6) δ ppm: 10.86 (s, 1H), 8.56 (d, J=8.2 Hz, 1H), 8.32 (d, J=5.3 Hz, 1H), 7.97 (s, 1H), 7.65 (d, J=2.4 Hz, 1H), 7.51 (d, J=2.0 Hz, 1H), 7.05 (dd, J=5.3, 1.6 Hz, 1H), 6.97 (d, J=2.0 Hz, 1H), 5.35 (hept, J=6.5 Hz, 1H), 5.03 (dd, J=8.0, 5.1 Hz, 1H), 4.75-4.68 (m, 1H), 4.49-4.39 (m, 1H), 3.87-3.71 (m, 3H, overlapping water), 3.33-3.26 (m, 4H), 2.28-2.16 (m, 1H), 2.14-1.93 (m, 2H), 1.86-1.59 (m, 4H), 1.47-1.18 (m, 8H).

### Example 310: N-((1S)-1-(4-chlorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-1-ethyl-1H-pyrazole-5-carboxamide

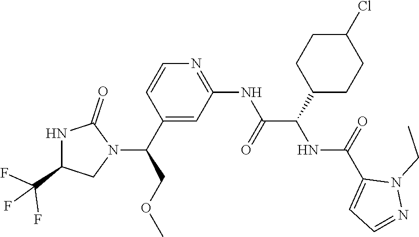 [SMILES: CCn1nccc1C(=O)N[C@H](C(=O)Nc1cc([C@@H](COC)N2C[C@@H](C(F)(F)F)NC2=O)ccn1)C1CCC(Cl)CC1]

Following the procedure described for Ex. 33 using 26 mg (S)-2-amino-2-((S)-3,3-difluorocyclohexyl)-N-(4-((S)-2-methoxy-1-((S)-2-oxo-4-(trifluoromethyl)imidazolidin-1-yl)ethyl)pyridin-2-yl)acetamide (Intermediate 75), 26 mg of the title compound was obtained. LC/MS: m/z=600.3 [M+H]⁺; rt: 1.96 min (LC/MS-method A). ¹H NMR (400 MHz, DMSO-d6) δ ppm: 10.78 (s, 1H), 8.54 (d, J=8.1 Hz, 1H), 8.31 (d, J=5.3 Hz, 1H), 8.00 (br s, 1H), 7.63 (d, J=2.4 Hz, 1H), 7.48 (d, J=2.0 Hz, 1H), 7.02 (dd, J=5.3, 1.6 Hz, 1H), 6.99 (d, J=2.0 Hz, 1H), 5.01 (dd, J=8.1, 5.0 Hz, 1H), 4.65-4.53 (m, 2H), 4.50-4.39 (m, 3H), 3.83 (dd, J=10.5, 8.1 Hz, 1H), 3.79-3.71 (m, 2H), 3.33-3.27 (m, 4H), 2.00-1.87 (m, 3H), 1.85-1.61 (m, 4H), 1.59-1.41 (m, 2H), 1.27 (t, J=7.1 Hz, 3H).

Examples 311-312 were prepared analogously to Example 310. Examples 313-317 were prepared analogously to Example 310 using the procedure described for Example 39.

| Ex. | Acid | Structure | Name | Yield |
|---|---|---|---|---|
| 311 | | | N-((1S)-1-(4- | 29 mg, |
| | | | chlorocyclohexyl)-2-((4-((S)- | 91% |
| | | | 2-methoxy-1-((S)-2-oxo-4- | |
| | | | (trifluoromethyl)imidazolidin- | |
| | | | 1-yl)ethyl)pyridin-2- | |
| | | | yl)amino)-2-oxoethyl)-1- | |
| | | | methyl-1H-pyrazole-5- | |
| | | | carboxamide | |
| 312 | | | N-((1S)-1-(4- | 24 mg, |
| | | | chlorocyclohexyl)-2-((4-((S)- | 71% |
| | | | 2-methoxy-1-((S)-2-oxo-4- | |
| | | | (trifluoromethyl)imidazolidin- | |
| | | | 1-yl)ethyl)pyridin-2- | |
| | | | yl)amino)-2-oxoethyl)-1- | |
| | | | isopropyl-1H-pyrazole-5- | |
| | | | carboxamide | |
| 313 | | | N-((1S)-1-(4- | 16 mg, |
| | | | chlorocyclohexyl)-2-((4-((S)- | 50% |
| | | | 2-methoxy-1-((S)-2-oxo-4- | |
| | | | (trifluoromethyl)imidazolidin- | |
| | | | 1-yl)ethyl)pyridin-2- | |
| | | | yl)amino)-2-oxoethyl)-3- | |
| | | | methylisoxazole-4- | |
| | | | carboxamide | |
| 314 | | | N-((1S)-1-(4- | 27 mg, |
| | | | chlorocyclohexyl)-2-((4-((S)- | 82% |
| | | | 2-methoxy-1-((S)-2-oxo-4- | |
| | | | (trifluoromethyl)imidazolidin- | |
| | | | 1-yl)ethyl)pyridin-2- | |
| | | | yl)amino)-2-oxoethyl)-4- | |
| | | | ethylisoxazole-3- | |
| | | | carboxamide | |
| 315 | | | N-((1S)-1-(4- | 17 mg, |
| | | | chlorocyclohexyl)-2-((4-((S)- | 53% |
| | | | 2-methoxy-1-((S)-2-oxo-4- | |
| | | | (trifluoromethyl)imidazolidin- | |
| | | | 1-yl)ethyl)pyridin-2- | |
| | | | yl)amino)-2-oxoethyl)-4- | |
| | | | methyl-1,2,5-oxadiazole-3- | |
| | | | carboxamide | |
| 316 | | | N-((1S)-1-(4- | 20 mg, |
| | | | chlorocyclohexyl)-2-((4-((S)- | 61% |
| | | | 2-methoxy-1-((S)-2-oxo-4- | |
| | | | (trifluoromethyl)imidazolidin- | |
| | | | 1-yl)ethyl)pyridin-2- | |
| | | | yl)amino)-2-oxoethyl)-4- | |
| | | | ethyl-1,2,5-oxadiazole-3- | |
| | | | carboxamide | |
| 317 | | | N-((1S)-1-(4- | 14 mg, |
| | | | chlorocyclohexyl)-2-((4-((S)- | 42% |
| | | | 2-methoxy-1-((S)-2-oxo-4- | |
| | | | (trifluoromethyl)imidazolidin- | |
| | | | 1-yl)ethyl)pyridin-2- | |
| | | | yl)amino)-2-oxoethyl)-3- | |
| | | | ethylisoxazole-4- | |
| | | | carboxamide | |

### Example 311

LC/MS: m/z=584.2 [M+H]⁺; rt: 1.85 min (LC/MS-method A). ¹H NMR (400 MHz, DMSO-d6) δ ppm: 10.82 (s, 1H), 8.54 (d, J=8.1 Hz, 1H), 8.31 (d, J=5.3 Hz, 1H), 8.00 (br s, 1H), 7.64 (d, J=2.3 Hz, 1H), 7.47 (d, J=2.0 Hz, 1H), 7.05-7.01 (m, 2H), 5.01 (dd, J=8.0, 5.1 Hz, 1H), 4.64-4.54 (m, 2H), 4.49-4.39 (m, 1H), 4.02 (s, 3H), 3.83 (dd, J=10.5, 8.1 Hz, 1H), 3.78-3.71 (m, 2H), 3.33-3.27 (m, 4H), 2.00-1.87 (m, 3H), 1.85-1.62 (m, 4H), 1.59-1.41 (m, 2H)

### Example 312

LC/MS: m/z=614.3 [M+H]⁺; rt: 2.08 min (LC/MS-method A). ¹H NMR (400 MHz, DMSO-d6) δ ppm: 10.80 (s, 1H), 8.53 (d, J=8.0 Hz, 1H), 8.31 (d, J=5.3 Hz, 1H), 8.00 (br s, 1H), 7.64 (d, J=2.3 Hz, 1H), 7.50 (d, J=2.0 Hz, 1H), 7.03 (dd, J=5.3, 1.6 Hz, 1H), 6.93 (d, J=2.0 Hz, 1H), 5.38 (hept, J=6.5 Hz, 1H), 5.02 (dd, J=8.1, 5.1 Hz, 1H), 4.63-4.53 (m, 2H), 4.49-4.39 (m, 1H), 3.83 (dd, J=10.6, 8.1 Hz, 1H), 3.79-3.71 (m, 2H), 3.33-3.27 (m, 4H), 2.00-1.87 (m, 3H), 1.85-1.62 (m, 4H), 1.59-1.41 (m, 2H), 1.36 (d, J=6.6 Hz, 3H), 1.34 (d, J=6.6 Hz, 3H)

### Example 313

LC/MS: m/z=587.2 [M+H]⁺; rt: 1.90 min (LC/MS-method A). ¹H NMR (400 MHz, DMSO-d6) δ ppm: 10.83 (s, 1H), 9.43 (s, 1H), 8.50 (d, J=8.0 Hz, 1H), 8.31 (d, J=5.3 Hz, 1H), 8.00 (br s, 1H), 7.63 (d, J=2.4 Hz, 1H), 7.03 (dd, J=5.3, 1.6 Hz, 1H), 5.01 (dd, J=8.1, 5.1 Hz, 1H), 4.67-4.59 (m, 1H), 4.58-4.53 (m, 1H), 4.49-4.39 (m, 1H), 3.83 (dd, J=10.5, 8.1 Hz, 1H), 3.79-3.71 (m, 2H), 3.33-3.25 (m, 4H), 2.37 (s, 3H), 1.98-1.40 (m, 9H)

### Example 314

LC/MS: m/z=601.3 [M+H]⁺; rt: 2.16 min (LC/MS-method A). ¹H NMR (400 MHz, DMSO-d6) δ ppm: 10.83 (s, 1H), 8.87 (t, J=0.9 Hz, 1H), 8.68 (d, J=8.3 Hz, 1H), 8.31 (d, J=5.3 Hz, 1H), 8.00 (br s, 1H), 7.64 (d, J=2.3 Hz, 1H), 7.03 (dd, J=5.3, 1.6 Hz, 1H), 5.02 (dd, J=8.1, 5.1 Hz, 1H), 4.69-4.62 (m, 1H), 4.57-4.53 (m, 1H), 4.50-4.39 (m, 1H), 3.83 (dd, J=10.5, 8.1 Hz, 1H), 3.78-3.70 (m, 2H, partially overlapping water), 3.33-3.28 (m, 4H), 2.59-2.52 (m, 2H), 2.00-1.86 (m, 3H), 1.85-1.74 (m, 2H), 1.72-1.60 (m, 2H), 1.58-1.43 (m, 2H), 1.13 (t, J=7.5 Hz, 3H)

### Example 315

LC/MS: m/z=588.2 [M+H]⁺; rt: 2.10 min (LC/MS-method A). ¹H NMR (400 MHz, DMSO-d6) δ ppm: 10.87 (s, 1H), 9.19 (d, J=8.2 Hz, 1H), 8.32 (d, J=5.3 Hz, 1H), 8.00 (br s, 1H), 7.64 (d, J=2.3 Hz, 1H), 7.03 (dd, J=5.3, 1.6 Hz, 1H), 5.02 (dd, J=8.1, 5.0 Hz, 1H), 4.72-4.66 (m, 1H), 4.59-4.54 (m, 1H), 4.50-4.39 (m, 1H), 3.83 (dd, J=10.5, 8.1 Hz, 1H), 3.79-3.72 (m, 2H), 3.33-3.28 (m, 4H), 2.48 (s, 3H), 2.02-1.87 (m, 3H), 1.85-1.75 (m, 2H), 1.74-1.62 (m, 2H), 1.60-1.43 (m, 2H)

### Example 316

LC/MS: m/z=602.3 [M+H]⁺; rt: 2.24 min (LC/MS-method A). ¹H NMR (400 MHz, DMSO-d6) δ ppm: 10.86 (s, 1H), 9.22 (d, J=8.1 Hz, 1H), 8.31 (d, J=5.3 Hz, 1H), 8.00 (br s, 1H), 7.64 (d, J=2.3 Hz, 1H), 7.03 (dd, J=5.3, 1.6 Hz, 1H), 5.02 (dd, J=8.1, 5.1 Hz, 1H), 4.73-4.67 (m, 1H), 4.58-4.54 (m, 1H), 4.50-4.39 (m, 1H), 3.83 (dd, J=10.5, 8.1 Hz, 1H), 3.79-3.71 (m, 2H), 3.34-3.27 (m, 4H), 2.91 (q, J=7.5 Hz, 2H), 2.01-1.87 (m, 3H), 1.85-1.62 (m, 4H), 1.60-1.42 (m, 2H), 1.24 (t, J=7.5 Hz, 3H)

### Example 317

LC/MS: m/z=601.3 [M+H]⁺; rt: 2.03 min (LC/MS-method A). ¹H NMR (400 MHz, DMSO-d6) δ ppm: 10.80 (s, 1H), 9.40 (s, 1H), 8.51 (d, J=8.1 Hz, 1H), 8.31 (d, J=5.3 Hz, 1H), 8.00 (br s, 1H), 7.63 (d, J=2.3 Hz, 1H), 7.02 (dd, J=5.3, 1.6 Hz, 1H), 5.01 (dd, J=8.1, 5.1 Hz, 1H), 4.66-4.60 (m, 1H), 4.58-4.53 (m, 1H), 4.49-4.39 (m, 1H), 3.83 (dd, J=10.5, 8.1 Hz, 1H), 3.78-3.71 (m, 2H), 3.32-3.26 (m, 4H), 2.83 (q, J=7.5 Hz, 2H), 1.99-1.40 (m, 9H), 1.17 (t, J=7.5 Hz, 3H)

### Example 318: N—((S)-1-((1R,3s,5S)-bicyclo[3.1.0]hexan-3-yl)-2-((4-((S)-2-methoxy-1-((S)-2-oxo-4-(trifluoromethyl)imidazolidin-1-yl)ethyl)pyridin-2-yl)amino)-2-oxoethyl)-5-ethylisoxazole-4-carboxamide

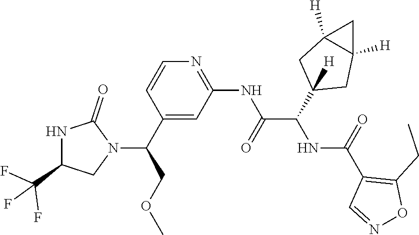

Following the procedure described for Ex. 39 using 60 mg (S)-2-amino-2-((1R,3s,5S)-bicyclo[3.1.0]hexan-3-yl)-N-(4-((S)-2-methoxy-1-((S)-2-oxo-4-(trifluoro-methyl)imidazolidin-1-yl)ethyl)pyridin-2-yl)acetamide (Intermediate 76), 22 mg of the title compound was obtained. LC/MS: m/z=565.2 [M+H]⁺; rt: 2.01 min (LC/MS-method A). ¹H NMR (400 MHz, DMSO-d6) δ ppm: 10.61 (s, 1H), 9.05 (s, 1H), 8.37 (d, J=7.6 Hz, 1H), 8.30 (d, J=5.2 Hz, 1H), 7.97 (br s, 1H), 7.62 (d, J=2.3 Hz, 1H), 7.00 (dd, J=5.2, 1.6 Hz, 1H), 5.01 (dd, J=8.1, 5.1 Hz, 1H), 4.55-4.38 (m, 2H), 3.82 (dd, J=10.5, 8.1 Hz, 1H), 3.78-3.71 (m, 2H), 3.32-3.26 (m, 4H), 3.06 (q, J=7.6 Hz, 2H), 2.08-1.91 (m, 2H), 1.81-1.71 (m, 1H), 1.68-1.57 (m, 1H), 1.56-1.46 (m, 1H), 1.29-1.22 (m, 2H), 1.19 (t, J=7.6 Hz, 3H), 0.34-0.26 (m, 1H), 0.20-0.14 (m, 1H).

### Example 319: N—((S)-1-((1R,3s,5S)-bicyclo[3.1.0]hexan-3-yl)-2-((4-((S)-2-methoxy-1-((S)-2-oxo-4-(trifluoromethyl)imidazolidin-1-yl)ethyl)pyridin-2-yl)amino)-2-oxoethyl)-5-isopropylisoxazole-4-carboxamide

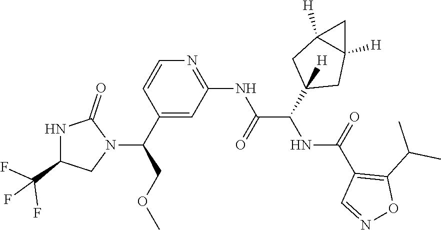

Prepared analogously to Example 318 affording 18 mg of the title compound. LC/MS: m/z=579.3 [M+H]⁺; rt: 2.14 min (LC/MS-method A). ¹H NMR (400 MHz, DMSO-d6) δ ppm: 10.61 (s, 1H), 9.04 (s, 1H), 8.37 (d, J=7.6 Hz, 1H), 8.30 (d, J=5.2 Hz, 1H), 7.97 (br s, 1H), 7.62 (d, J=2.4 Hz, 1H), 7.00 (dd, J=5.2, 1.6 Hz, 1H), 5.01 (dd, J=8.2, 5.0 Hz, 1H), 4.55-4.38 (m, 2H), 3.86-3.71 (m, 3H), 3.60-3.50 (m, 1H, overlapping water), 3.33-3.25 (m, 4H), 2.06-1.91 (m, 2H), 1.81-1.71 (m, 1H), 1.65-1.58 (m, 1H), 1.55-1.46 (m, 1H), 1.29-1.19 (m, 8H), 0.33-0.26 (m, 1H), 0.20-0.14 (m, 1H).

### Example 320: 1-isopropyl-N—((S)-1-((1r,4S)-4-methylcyclohexyl)-2-oxo-2-((4-(((3S,5S)-2-oxo-5-(trifluoromethyl)pyrrolidin-3-yl)oxy)pyridin-2-yl)amino)ethyl)-1H-pyrazole-5-carboxamide

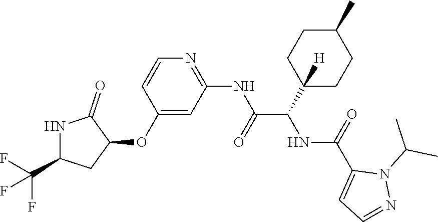

Following the procedure described for Ex. 33 using 42 mg ((S)-2-amino-2-((1r,4S)-4-methylcyclohexyl)-N-(4-(((3S,5S)-2-oxo-5-(trifluoromethyl)pyrrolidin-3-yl)oxy)pyridin-2-yl)acetamide (Intermediate 77), 32 mg of the title compound was obtained. LC/MS: m/z=551.2 [M+H]⁺; rt: 2.10 min (LC/MS-method A). ¹H NMR (400 MHz, DMSO-d6) δ ppm: 11.41 (br s, 1H), 9.28 (br s, 1H), 8.60 (d, J=7.7 Hz, 1H), 8.22 (d, J=6.4 Hz, 1H), 7.59 (d, J=2.5 Hz, 1H), 7.50 (d, J=2.0 Hz, 1H), 7.08 (dd, J=6.4, 2.5 Hz, 1H), 6.97 (d, J=2.0 Hz, 1H), 5.42-5.29 (m, 2H), 4.55-4.41 (m, 2H), 2.80-2.39 (m, 2H, overlapping solvent), 1.91-1.78 (m, 2H), 1.75-1.65 (m, 2H), 1.65-1.55 (m, 1H), 1.40-1.17 (m, 8H), 1.16-1.02 (m, 1H), 0.96-0.80 (m, 5H)

### Example 321: 3-ethyl-N—((S)-1-((1r,4S)-4-methylcyclohexyl)-2-oxo-2-((4-(((3S,5S)-2-oxo-5-(trifluoromethyl)pyrrolidin-3-yl)oxy)pyridin-2-yl)amino)ethyl)isoxazole-4-carboxamide

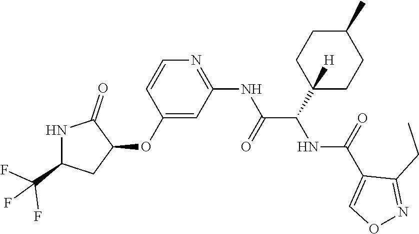

Prepared analogously to Example 320 affording 29 mg of the title compound. LC/MS: m/z=538.2 [M+H]⁺; rt: 2.12 min (LC/MS-method A). ¹H NMR (400 MHz, DMSO-d6) δ ppm: 11.17 (br s, 1H), 9.43 (s, 1H), 9.26 (br s, 1H), 8.53 (d, J=7.8 Hz, 1H), 8.20 (d, J=6.2 Hz, 1H), 7.63 (d, J=2.5 Hz, 1H), 7.02 (dd, J=6.2, 2.5 Hz, 1H), 5.32-5.25 (m, 1H), 4.54-4.43 (m, 2H), 2.82 (q, J=7.5 Hz, 2H), 2.79-2.39 (m, 2H, overlapping solvent), 1.88-1.54 (m, 5H), 1.36-1.02 (m, 6H), 0.95-0.80 (m, 5H).

### Example 322: 1-ethyl-N—((S)-1-((1r,4S)-4-methylcyclohexyl)-2-oxo-2-((4-(((3S,5S)-2-oxo-5-(trifluoromethyl)pyrrolidin-3-yl)oxy)pyridin-2-yl)amino)ethyl)-1H-pyrazole-5-carboxamide

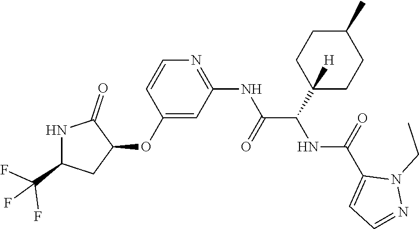

Prepared analogously to Example 320 affording 30 mg of the title compound. LC/MS: m/z=537.2 [M+H]⁺; rt: 2.07 min (LC/MS-method A). ¹H NMR (400 MHz, DMSO-d6) δ ppm: 11.29 (br s, 1H), 9.27 (br s, 1H), 8.59 (d, J=7.8 Hz, 1H), 8.21 (d, J=6.4 Hz, 1H), 7.60 (d, J=2.5 Hz, 1H), 7.49 (d, J=2.1 Hz, 1H), 7.06 (dd, J=6.4, 2.5 Hz, 1H), 7.03 (d, J=2.1 Hz, 1H), 5.36-5.27 (m, 1H), 4.55-4.40 (m, 4H), 2.79-2.39 (m, 2H, overlapping solvent), 1.90-1.77 (m, 2H), 1.74-1.65 (m, 2H), 1.64-1.55 (m, 1H), 1.37-1.17 (m, 5H), 1.15-1.01 (m, 1H), 0.95-0.80 (m, 5H).

### Example 323: 1-isopropyl-N—((S)-1-((1r,4S)-4-methylcyclohexyl)-2-oxo-2-((4-(((3R,5S)-2-oxo-5-(trifluoromethyl)pyrrolidin-3-yl)oxy)pyridin-2-yl)amino)ethyl)-1H-pyrazole-5-carboxamide

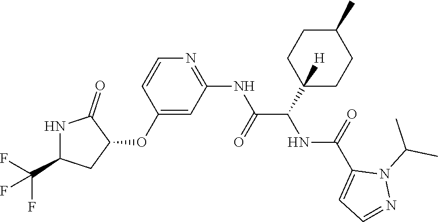

Following the procedure described for Example 33, using 37 mg ((S)-2-amino-2-((1r,4S)-4-methylcyclohexyl)-N-(4-(((3R,5S)-2-oxo-5-(trifluoromethyl)pyrrolidin-3-yl)oxy)pyridin-2-yl)acetamide (Intermediate 78), 28 mg of the title compound was obtained. LC/MS: m/z=551.2 [M+H]⁺; rt: 2.21 min (LC/MS-method A). ¹H NMR (400 MHz, DMSO-d6) δ ppm: 10.77 (s, 1H), 9.21 (d, J=8.0 Hz, 1H), 8.51 (d, J=7.9 Hz, 1H), 8.26 (d, J=5.2 Hz, 1H), 8.00 (br s, 1H), 7.07 (dd, J=5.2, 1.5 Hz, 1H), 4.74-4.69 (m, 1H), 4.68-4.62 (m, 1H), 2.90 (q, J=7.5 Hz, 2H), 2.53-2.37 (m, 4H), 2.10-1.99 (m, 3H), 1.91-1.61 (m, 6H), 1.53-1.44 (m, 1H), 1.41-1.32 (m, 1H), 1.24 (t, J=7.5 Hz, 3H), 0.87 (t, J=7.3 Hz, 3H).

### Biological Assays

The biological activity of the compounds of the present disclosure was determined utilising the assays described herein. Activity is reported as IC₅₀ in μM. These values may fluctuate depending on the daily assay performance, fluctuations of this kind are known to those skilled in the art. These results show that the compounds of the present disclosure are capable of inhibiting the biological action of IL-17A.

In general, the compounds of the present invention possess IC₅₀ values of 30 μM or less against IL-17A in the assays described herein. Suitably, the compounds of the present invention possess IC₅₀ values of 10 μM or less (such as 1 μM or less, 500 nM or less, or 250 nM or less) against IL-17A in the assays described herein.

The compounds of Formula I, which comprise a defined substitution pattern in the central six-membered heteroaryl ring (e.g. 2,4-pyridyl) and an R⁵ group attached to this ring via a Z linker group, are unexpectedly potent at inhibiting IL-17A.

### IL-17A AlphaLISA Assay

The ability of the compounds to block binding of IL-17A to its receptor, IL-17RA, was analysed in a competition assay using AlphaLISA technology (Perkin Elmer). The assay is a bead based AlphaLISA where the IL-17RA is captured on the acceptor bead via an Fc tag and IL-17A is captured on the streptavidin donor bead via a biotinylated anti-IL17A antibody.

Assay buffer was prepared by adding 0.05% Tween-20 (v/v) and 0.1% BSA to Phosphate Buffered Saline (PBS). The assay was carried out in 384-well white low volume plates (Perkin Elmer #6008359). 10 μL of a 7.5 nM stock of human recombinant IL-17A (R&D Systems 7955-IL/CF) diluted in assay buffer was dispensed into the assay plate and compounds or DMSO vehicle control were added in a volume of 75 nL using an acoustic dispenser (Beckman). The compounds were pre-incubated with the IL-17A for 30 min at room temperature prior to addition of 5 μL of a 5 nM stock of human recombinant IL-17RA/Fc chimera (R&D Systems 177-IR-100) diluted in assay buffer. The IL-17A was incubated with the receptor for a further 90 minutes at room temperature before addition of 5 μL of a mixture of anti-human Fc IgG acceptor beads (75 μg/mL, Perkin Elmer AL1030) and anti-IL-17A biotin conjugated antibody (5 nM, Abcam, #ab155575) in assay buffer. After a further 30 min incubation at room temperature, 5 μL of streptavidin donor beads (75 μg/mL, Perkin Elmer 6760002S) were added and the plate was incubated for 3 h in the dark.

The AlphaLisa signal was measured using the PheraSTAR plate reader (BMG). Data were analysed using GeneData Screener and fitted to a 4-parameter logistic equation. The IC₅₀ values were calculated using the DMSO vehicle as the negative control and a high concentration (4 μM) of a reference IL-17A inhibitor as the positive control. All compounds were analysed at least in duplicate (n≥2).

Activity of the Example compounds is reported in Table A as the mean IC₅₀ values.

TABLE A

| Ex. | IC₅₀ | Ex. | IC₅₀ | Ex. | IC₅₀ | Ex. | IC₅₀ | Ex. | IC₅₀ |
|---|---|---|---|---|---|---|---|---|---|
| 1 | 0.148 | 2 | 0.025 | 3 | 0.085 | 4 | 0.035 | 5 | 0.179 |
| 6 | 0.566 | 7 | 0.407 | 8 | 0.141 | 9 | 0.422 | 10 | 0.273 |
| 11 | 0.359 | 12 | 0.106 | 13 | 0.122 | 14 | 0.333 | 15 | 0.201 |
| 16 | 0.157 | 17 | 0.092 | 18 | 0.448 | 19 | 0.843 | 20 | 0.183 |
| 21 | 0.138 | 22 | 9.23 | 23 | 6.95 | 24 | 1.05 | 25 | 0.105 |
| 26 | 10.4 | 27 | 14.9 | 28 | 8.40 | 29 | 0.428 | 30 | 0.479 |
| 31 | 5.06 | 32 | 5.60 | 33 | 1.01 | 34 | 1.47 | 35 | 23.5 |
| 36 | 12.0 | 37 | 25.4 | 38 | 0.448 | 39 | 0.138 | 40 | 0.092 |
| 41 | 0.282 | 42 | 0.524 | 43 | 0.171 | 44 | 0.151 | 45 | 10.3 |
| 46 | 0.612 | 47 | 0.630 | 48 | 0.229 | 49 | 0.776 | 50 | 2.95 |
| 51 | 2.08 | 52 | 23.9 | 53 | 3.88 | 54 | 4.76 | 55 | 0.183 |
| 56 | 7.42 | 57 | 1.96 | 58 | 0.011 | 59 | 0.061 | 60 | 3.75 |
| 61 | 0.612 | 62 | 1.40 | 63 | 0.330 | 64 | 1.23 | 65 | 0.522 |
| 66 | 0.074 | 67 | 0.809 | 68 | 0.136 | 69 | 0.126 | 70 | 8.00 |
| 71 | 0.197 | 72 | 0.547 | 73 | 1.01 | 74 | 2.62 | 75 | 0.134 |
| 76 | 0.093 | 77 | 0.060 | 78 | 0.069 | 79 | 5.27 | 80 | 1.24 |
| 81 | 6.14 | 82 | 1.17 | 83 | 7.11 | 84 | 0.373 | 85 | 0.012 |
| 86 | 0.006 | 87 | 0.013 | 88 | 0.004 | 89 | 0.011 | 90 | 4.52 |
| 91 | 0.119 | 92 | 0.539 | 93 | 0.341 | 94 | 4.86 | 95 | 5.49 |
| 96 | 0.096 | 97 | 2.08 | 98 | 1.96 | 99 | 6.79 | 100 | 17.4 |
| 101 | 5.39 | 102 | 0.356 | 103 | 1.74 | 104 | 0.938 | 105 | 3.34 |
| 106 | 0.031 | 107 | 0.177 | 108 | 0.033 | 109 | 7.80 | 110 | 0.030 |
| 111 | 6.17 | 112 | 0.381 | 113 | 1.00 | 114 | 0.082 | 115 | 0.077 |
| 116 | 15.4 | 117 | 0.723 | 118 | 8.97 | 119 | 3.99 | 120 | 0.018 |
| 121 | 0.023 | 122 | 0.082 | 123 | 0.065 | 124 | 0.020 | 125 | 0.013 |
| 126 | 0.021 | 127 | 0.440 | 128 | 2.35 | 129 | 0.533 | 130 | 0.350 |
| 131 | 0.680 | 132 | 7.70 | 133 | 0.073 | 134 | 1.25 | 135 | 1.46 |
| 136 | 0.252 | 137 | 0.197 | 138 | 0.164 | 139 | 0.265 | 140 | 7.37 |
| 141 | 0.646 | 142 | 0.203 | 143 | 3.23 | 144 | 1.83 | 145 | 1.36 |
| 146 | 4.45 | 147 | 0.828 | 148 | 8.28 | 149 | 0.108 | 150 | 0.061 |
| 151 | 0.401 | 152 | 2.03 | 153 | 0.171 | 154 | 0.527 | 155 | 0.029 |
| 156 | 0.053 | 157 | 0.117 | 158 | 0.018 | 159 | 0.030 | 160 | 0.183 |
| 161 | 0.615 | 162 | 0.444 | 163 | 0.020 | 164 | 0.035 | 165 | 0.041 |
| 166 | 0.050 | 167 | 0.222 | 168 | 5.79 | 169 | 0.079 | 170 | 0.021 |
| 171 | 0.067 | 172 | 1.17 | 173 | 0.187 | 174 | 0.362 | 175 | 0.384 |
| 176 | 0.057 | 177 | 0.024 | 178 | 0.053 | 179 | 0.083 | 180 | 0.073 |
| 181 | 0.102 | 182 | 0.019 | 183 | 0.042 | 184 | 0.637 | 185 | 0.046 |
| 186 | 0.069 | 187 | 0.017 | 188 | 0.031 | 189 | 0.065 | 190 | 0.816 |
| 191 | 0.031 | 192 | 0.105 | 193 | 0.105 | 194 | 0.068 | 195 | 0.018 |
| 196 | 0.039 | 197 | 0.056 | 198 | 0.047 | 199 | 0.033 | 200 | 0.018 |
| 201 | 0.070 | 202 | 0.070 | 203 | 0.090 | 204 | 0.129 | 205 | 0.347 |
| 206 | 0.157 | 207 | 0.350 | 208 | 0.031 | 209 | 0.023 | 210 | 0.038 |
| 211 | 0.033 | 212 | 0.023 | 213 | 0.028 | 214 | 0.046 | 215 | 0.065 |
| 216 | 0.137 | 217 | 0.465 | 218 | 0.158 | 219 | 0.080 | 220 | 0.048 |
| 221 | 0.157 | 222 | 0.070 | 223 | 0.084 | 224 | 0.135 | 225 | 0.105 |
| 226 | 0.042 | 227 | 0.020 | 228 | 0.062 | 229 | 0.086 | 230 | 0.096 |
| 231 | 0.038 | 232 | 0.053 | 233 | 0.009 | 234 | 0.039 | 235 | 0.066 |
| 236 | 0.026 | 237 | 0.022 | 238 | 0.014 | 239 | 0.016 | 240 | 0.016 |
| 241 | 0.029 | 242 | 0.085 | 243 | 0.038 | 244 | 0.047 | 245 | 0.073 |
| 246 | 0.072 | 247 | 0.058 | 248 | 0.054 | 249 | 0.049 | 250 | 0.038 |
| 251 | 0.033 | 252 | 0.049 | 253 | 0.106 | 254 | 0.077 | 255 | 0.071 |
| 256 | 0.034 | 257 | 0.032 | 258 | 0.009 | 259 | 0.006 | 260 | 0.013 |
| 261 | 0.189 | 262 | 0.198 | 263 | 0.090 | 264 | 0.099 | 265 | 0.045 |
| 266 | 0.015 | 267 | 0.027 | 268 | 0.031 | 269 | 0.010 | 270 | 0.029 |
| 271 | 0.036 | 272 | 0.048 | 273 | 0.403 | 274 | 0.075 | 275 | 0.946 |
| 276 | 0.024 | 277 | 0.044 | 278 | 1.89 | 279 | 12.0 | 280 | 0.065 |
| 281 | 0.117 | 282 | 0.103 | 283 | 0.097 | 284 | 0.062 | 285 | 0.017 |
| 286 | 0.040 | 287 | 0.047 | 288 | 0.084 | 289 | 0.125 | 290 | 0.160 |
| 291 | 0.619 | 292 | 0.104 | 293 | 0.153 | 294 | 0.374 | 295 | 0.025 |
| 296 | 4.49 | 297 | 4.80 | 298 | 0.085 | 299 | 0.072 | 300 | 0.039 |
| 301 | 0.038 | 302 | 0.044 | 303 | 0.046 | 304 | 0.026 | 305 | 0.029 |
| 306 | 0.047 | 307 | 0.051 | 308 | 0.074 | 309 | 0.098 | 310 | 0.071 |
| 311 | 0.096 | 312 | 0.121 | 313 | 0.109 | 314 | 0.058 | 315 | 0.090 |
| 316 | 0.046 | 317 | 0.019 | 318 | 0.064 | 319 | 0.038 | 320 | 0.604 |
| 321 | 0.689 | 322 | 0.821 | 323 | 1.21 | | | | |

While particular embodiments of the present disclosure have been shown and described herein, it will be obvious to those skilled in the art that such embodiments are provided by way of example only. Numerous variations, changes, and substitutions will occur to those skilled in the art without departing from the disclosure. It should be understood that various alternatives to the embodiments of the disclosure described herein may be employed in practising the disclosure. It is intended that the following claims define the scope of the disclosure and that methods and structures within the scope of these claims and their equivalents be covered thereby. The disclosures of all patent and scientific literature cited herein are expressly incorporated herein in their entirety by reference. To the extent that any incorporated material is inconsistent with the express content of this disclosure, the express content controls.

1. A compound of Formula (I) or a pharmaceutically acceptable salt thereof: 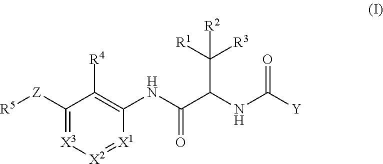
wherein:
Z is O or CR⁶R⁷;
at least one of X¹, X², and X³ is N and the others are CR⁴;
Y is selected from 5- or 6-membered monocyclic heteroaryl, 9- or 10-membered bicyclic heteroaryl, 5- or 6-membered heteroaryl fused to 5- or 6-membered cycloalkyl or 5- or 6-membered heterocyclyl, phenyl, heterocyclyl, C₃₋₇cycloalkyl, and QR⁸; wherein:
i. Q is absent, O, or NR⁹;
ii. R⁸ is C₁₋₆alkyl, C₁₋₃alkylene-phenyl, or C₁₋₃alkylene-heteroaryl;
iii. Y is optionally substituted with one or more substituents Y^{A}, wherein at each occurrence Y^{A} is independently selected from halo, oxo, C₁₋₄alkyl, hydroxy, C₁₋₄alkoxy, C₁₋₃haloalkyl, cyano, C(O)R¹⁰, and C₃₋₇cycloalkyl; and
iv. Y^{A} is optionally further substituted with one or more substituents independently selected from halo, C₁₋₄alkyl, hydroxy, C₁₋₄alkoxy, C₃₋₇cycloalkyl, C₁₋₃alkylene-NR¹¹R¹², and C₁₋₃haloalkyl;
R¹ and R² are independently selected from hydrogen, C₁₋₄alkyl, C₃₋₇cycloalkyl, C₁₋₅alkoxy, and phenyl, wherein said C₁₋₄alkyl, C₃₋₇cycloalkyl, C₁₋₅alkoxy, or phenyl is optionally substituted with one or more substituents independently selected from halo, C₁₋₄alkyl, and C₁₋₃haloalkyl; provided that R¹ and R² cannot be both hydrogen; OR
R¹ and R², taken together with the carbon atom to which they are attached, form a 4- to 10-membered cycloalkyl ring, wherein the cycloalkyl ring is optionally substituted with one or more substituents independently selected from halo, C₁₋₄alkyl, and C₁₋₃haloalkyl;
R³ is hydrogen, fluoro, or methyl;
R⁴ at each occurrence is independently selected from hydrogen, fluoro, methyl, hydroxy, and trifluoromethyl;
R⁵ is selected from 5- or 6-membered monocyclic heteroaryl, 4- to 10-membered heterocyclyl, C(O)NR¹³R¹⁴, OC(O)NR¹⁵R¹⁶, and NR¹⁷C(O)C₁₋₆alkyl, wherein R¹³, R¹⁴, R¹⁵, R¹⁶, and R¹⁷ are independently selected from hydrogen and C₁₋₆alkyl; and wherein R⁵ is optionally substituted with one or more substituents independently selected from hydroxy, halo, C₁₋₄alkyl, C₁₋₄alkoxy, C₁₋₃haloalkyl, C₃₋₇cycloalkyl, cyano, and oxo;
R⁶ is hydrogen, fluoro, C₁₋₆alkyl, or C₃₋₇cycloalkyl; wherein when R⁶ is C₁₋₆alkyl or C₃₋₇cycloalkyl, said groups are optionally substituted with one or more substituents independently selected from hydroxy, halo, C₁₋₄alkyl, C₁₋₄alkoxy, C₁₋₃haloalkyl, and C₁₋₃haloalkoxy;
R⁷ is selected from hydrogen, deuterium, halo, and C₁₋₄alkyl; OR
R⁶ and R⁷, together with the carbon atom to which they are attached, form a 3- to 6-membered cycloalkyl or heterocyclyl ring, optionally substituted with one or more substituents independently selected from halo, C₁₋₄alkyl, C₁₋₄alkoxy, and C₁₋₃haloalkyl;
R⁹ is hydrogen or C₁₋₄alkyl;
R¹⁰ is hydroxy, C₁₋₄alkyl, or C₁₋₄alkoxy; and
R¹¹ and R¹² are independently selected from hydrogen, C₁₋₄alkyl, and C(O)OC₁₋₄alkyl.

2. The compound according to claim 1, having a structure according to Formula IA, or a pharmaceutically acceptable salt thereof: 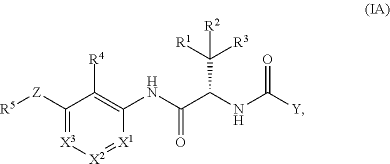
wherein Z, X¹, X², X³, Y, R¹, R², R³, R⁴, and R⁵ are as defined in claim 1.

3. The compound according to claim 1 or claim 2, wherein Z is CR⁶R⁷.

4. The compound according to any one of claims 1 to 3, wherein one of X¹, X², and X³ is N, and the others are CR⁴.

5. The compound according to claim 4, wherein X¹ is N, and X² and X³ are CR⁴.

6. The compound according to any one of claims 1 to 5, wherein Y is selected from 5- or 6-membered monocyclic heteroaryl, 5- or 6-membered heteroaryl fused to 5- or 6-membered cycloalkyl or 5- or 6-membered heterocyclyl, heterocyclyl, cyclopropyl, and QR⁸; wherein:
i. Q is absent, O, or NR⁹;
ii. R⁸ is C₁₋₆alkyl, C₁₋₃alkylene-phenyl, or C₁₋₃alkylene-heteroaryl;
iii. Y is optionally substituted with one or more substituents Y^{A}, wherein at each occurrence Y^{A} is independently selected from halo, C₁₋₄alkyl, C₁₋₃haloalkyl, cyano, C(O)R¹⁰, and C₃₋₇cycloalkyl; and
iv. Y^{A} is optionally further substituted with one or more substituents independently selected from halo, hydroxy, C₃₋₇cycloalkyl, and C₁₋₃alkylene-NR¹¹R¹².

7. The compound according to claim 6, wherein Y is QR⁸ wherein Q is absent or O, R⁸ is C₁₋₆alkyl, CH₂-phenyl, CH₂-heteroaryl, CF₂-phenyl, or CF₂-heteroaryl, and said phenyl and heteroaryl groups are optionally substituted with one or more substituents Y^{A}, wherein at each occurrence Y^{A} is independently selected from fluoro, chloro, C₁₋₄alkyl, C₁₋₃haloalkyl, cyano, C(O)R¹⁰, and cyclopropyl; and each Y^{A} is optionally further substituted with one or more substituents independently selected from halo, hydroxy, cyclopropyl, and C₁₋₃alkylene-NR¹¹R¹².

8. The compound according to claim 6, wherein Y is 5- or 6-membered monocyclic heteroaryl optionally substituted with one or more substituents Y^{A}, wherein at each occurrence Y^{A} is independently selected from fluoro, chloro, C₁₋₄alkyl, C₁₋₃haloalkyl, cyano, C(O)R¹⁰, and cyclopropyl, and wherein each Y^{A} is optionally further substituted with one or more substituents independently selected from halo, hydroxy, cyclopropyl, and C₁₋₃alkylene-NR¹¹R¹².

9. The compound according to claim 8, wherein Y is a 5- or 6-membered monocyclic heteroaryl selected from one of the following structures: 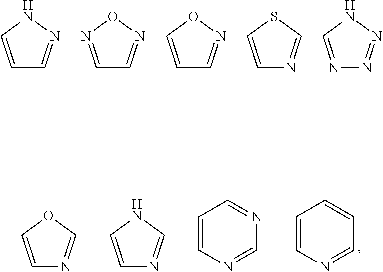
wherein the heteroaryl is optionally substituted with one or more substituents Y^{A} wherein at each occurrence Y^{A} is independently selected from fluoro, chloro, C₁₋₄alkyl, C₁₋₃haloalkyl, cyano, C(O)R¹⁰, and cyclopropyl, and wherein each Y^{A} is optionally further substituted with one or more substituents independently selected from halo, hydroxy, cyclopropyl, and C₁₋₃alkylene-NR¹¹R¹².

10. The compound according to any one of claims 1 to 9, wherein R¹ and R² are independently selected from hydrogen, C₁₋₄alkyl, C₃₋₇cycloalkyl, and C₁₋₅alkoxy, wherein said C₁₋₄alkyl, C₃₋₇cycloalkyl, or C₁₋₅alkoxy is optionally substituted with one or more substituents independently selected from halo, C₁₋₄alkyl, and C₁₋₃haloalkyl; provided that R¹ and R² cannot be both hydrogen.

11. The compound according to any one of claims 1 to 9, wherein R¹ and R² together with the carbon atom to which they are attached form a cyclohexyl ring, wherein the cyclohexyl ring is substituted with one or more substituents independently selected from halo, C₁₋₄alkyl, and C₁₋₃haloalkyl.

12. The compound according to any one of claims 1 to 9, wherein the combined R¹, R², and R³ group is a group selected from: 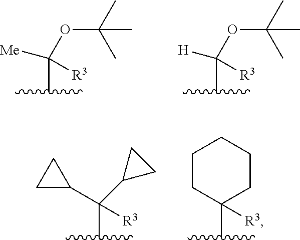
wherein [symbol] is the point of attachment to the rest of the compound of Formula I; and
each cyclopropyl or cyclohexyl ring is optionally substituted with one or more substituents independently selected from halo, C₁₋₄alkyl, and C₁₋₃haloalkyl.

13. The compound according to any one of claims 1 to 12, wherein R³ is hydrogen.

14. The compound according to any one of claims 1 to 13, wherein R⁴ at each occurrence is independently selected from hydrogen, fluoro, and methyl.

15. The compound according to claim 14, wherein R⁴ at each occurrence is independently selected from hydrogen and fluoro.

16. The compound according to any one of claims 1 to 15, wherein R⁵ is selected from 5- or 6-membered monocyclic heteroaryl, 4- to 10-membered heterocyclyl, C(O)NR¹³R¹⁴, OC(O)NR¹⁵R¹⁶, and NR¹⁷C(O)C₁₋₆alkyl, wherein R¹³, R¹⁴, R¹⁵, R¹⁶, and R¹⁷ are independently selected from hydrogen and C₁₋₆alkyl; and wherein R⁵ is optionally substituted with one or more substituents independently selected from halo, C₁₋₄alkyl, C₁₋₃haloalkyl, C₃₋₇cycloalkyl, and oxo.

17. The compound according to claim 16, wherein R⁵ is selected from 5-membered monocyclic heteroaryl, 5-membered heterocyclyl, and NR¹⁷C(O)C₁₋₆alkyl, wherein R¹⁷ is hydrogen or methyl; and wherein R⁵ is optionally substituted with one or more substituents independently selected from hydroxy, halo, C₁₋₄alkyl, C₁₋₃haloalkyl, C₃₋₇cycloalkyl, and oxo.

18. The compound according to any one of claims 1 to 15, wherein R⁵ has the structure: 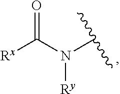
wherein [symbol] is the point of attachment to the rest of the compound of Formula I; R^{x} is C₁₋₆alkyl optionally substituted with one or more substituents independently selected from hydroxy, halo, C₁₋₄alkoxy, C₁₋₃haloalkyl, and C₃₋₇cycloalkyl; and R^{y} is hydrogen or C₁₋₆alkyl; or R^{x} and R^{y}, taken together with the atoms to which they are attached, form a 5- to 8-membered heterocyclic ring, optionally substituted with one or more substituents independently selected from hydroxy, halo, C₁₋₄alkyl, C₁₋₄alkoxy, C₁₋₃haloalkyl, C₃₋₇cycloalkyl, and oxo.

19. The compound according to claim 18, wherein R^{x} is C₁₋₄alkyl optionally substituted with one or more substituents independently selected from hydroxy, fluoro, methoxy, and cyclopropyl; and R^{y} is hydrogen or methyl; or R^{x} and R^{y}, taken together with the atoms to which they are attached, form a 5-membered heterocyclic ring, optionally substituted with one or more substituents independently selected from fluoro, C₁₋₄alkyl, trifluoromethyl, and oxo.

20. The compound according to claim 19, wherein R⁵ has the structure: 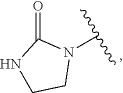
wherein [symbol] is the point of attachment to the rest of the compound of Formula I; and
the structure is optionally substituted with one or more substituents independently selected from fluoro, methyl, trifluoromethyl, and oxo.

21. The compound according to any one of claims 1 to 15, wherein R⁵ has a structure selected from: 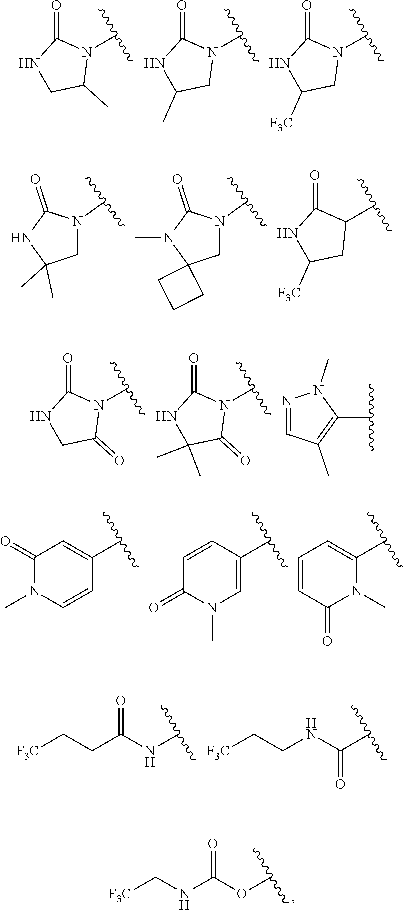
wherein [symbol] is the point of attachment to the rest of the compound of Formula I.

22. The compound according to any one of claims 1 to 21, wherein R⁶ is hydrogen, C₁₋₆alkyl, or C₃₋₇cycloalkyl; wherein when R⁶ is C₁₋₆alkyl or C₃₋₇cycloalkyl, said groups are optionally substituted with one or more substituents independently selected from hydroxy, halo, C₁₋₄alkyl, C₁₋₄alkoxy, and C₁₋₃haloalkyl.

23. The compound according to claim 22, wherein R⁶ is hydrogen, methyl, or cyclopropyl; wherein when R⁶ is methyl or cyclopropyl, said groups are optionally substituted with one or more substituents independently selected from fluoro and methoxy.

24. The compound according to any one of claims 1 to 23, wherein R⁷ is selected from hydrogen, deuterium, fluoro, and methyl.

25. The compound according to claim 24, wherein R⁷ is hydrogen.

26. The compound according to any one of claims 1 to 25, wherein R⁶ and R⁷, together with the carbon atom to which they are attached, form a 4- to 6-membered cycloalkyl ring, optionally substituted with one or more substituents independently selected from halo, C₁₋₄alkyl, C₁₋₄alkoxy, and C₁₋₃haloalkyl.

27. The compound according to claim 1, having a structure according to any one of Formulae IB to IE, or a pharmaceutically acceptable salt thereof: 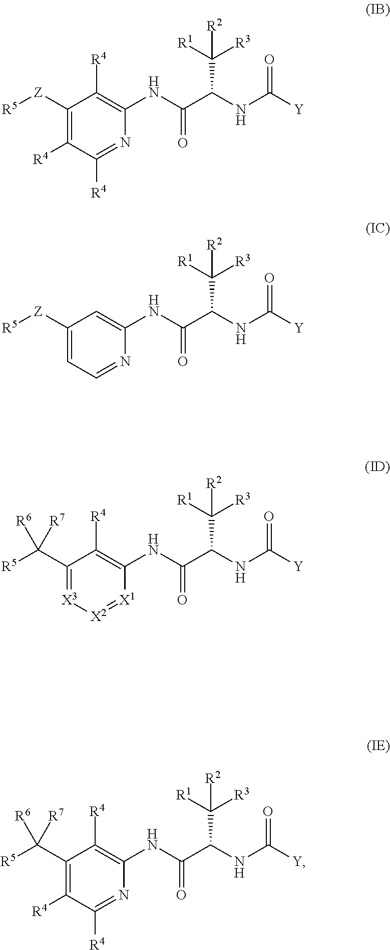
wherein Z, X¹, X², X³, Y, R¹, R², R³, R⁴, R⁵, R⁶, and R⁷ are as defined in any one of claims 1 to 26.

28. The compound according to claim 1, having a structure according to Formula IF, or a pharmaceutically acceptable salt thereof: 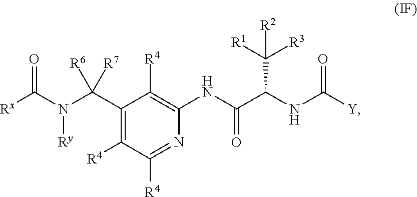
wherein Y, R¹, R², R³, R⁴, R⁶, and R⁷ are as defined in any one of claims 1 to 26; R^{x} is C₁₋₆alkyl optionally substituted with one or more substituents independently selected from hydroxy, halo, C₁₋₄alkoxy, C₁₋₃haloalkyl, and C₃₋₇cycloalkyl, and R^{y} is hydrogen or C₁₋₆alkyl; or R^{x} and R^{y}, taken together with the atoms to which they are attached, form a 5- to 8-membered heterocyclic ring, optionally substituted with one or more substituents independently selected from hydroxy, halo, C₁₋₄alkyl, C₁₋₄alkoxy, C₁₋₃haloalkyl, C₃₋₇cycloalkyl, and oxo.

29. The compound according to claim 1, wherein the compound is selected from one of the following compounds, or a pharmaceutically acceptable salt thereof:
Tert-butyl ((S)-1-((1r,4S)-4-methylcyclohexyl)-2-oxo-2-((4-(((S)-2-oxo-4-(trifluoromethyl)imidazolidin-1-yl)methyl)pyridin-2-yl)amino)ethyl)carbamate;
4-Cyclopropyl-N—((S)-1-((1r,4S)-4-methylcyclohexyl)-2-oxo-2-((4-(((S)-2-oxo-4-(trifluoromethyl)imidazolidin-1-yl)methyl)pyridin-2-yl)amino)ethyl)-1,2,5-oxadiazole-3-carboxamide;
1-Methyl-N—((S)-1-((1r,4S)-4-methylcyclohexyl)-2-oxo-2-((4-(((S)-2-oxo-4-(trifluoromethyl)imidazolidin-1-yl)methyl)pyridin-2-yl)amino)ethyl)-1H-pyrazole-5-carboxamide;
1-Isopropyl-N—((S)-1-((1r,4S)-4-methylcyclohexyl)-2-oxo-2-((4-(((S)-2-oxo-4-(trifluoromethyl)imidazolidin-1-yl)methyl)pyridin-2-yl)amino)ethyl)-1H-pyrazole-5-carboxamide;
Benzyl ((S)-1-((1r,4S)-4-methylcyclohexyl)-2-oxo-2-((4-(((S)-2-oxo-4-(trifluoromethyl)imidazolidin-1-yl)methyl)pyridin-2-yl)amino)ethyl)carbamate;
Tert-butyl ((S)-1-(4,4-difluorocyclohexyl)-2-oxo-2-((4-(((S)-2-oxo-4-(trifluoromethyl)imidazolidin-1-yl)methyl)pyridin-2-yl)amino)ethyl)carbamate;
Tert-butyl 6-(((S)-1-((1r,4S)-4-methylcyclohexyl)-2-oxo-2-((4-(((S)-2-oxo-4-(trifluoromethyl)imidazolidin-1-yl)methyl)pyridin-2-yl)amino)ethyl)carbamoyl)-3,4-dihydropyrrolo[1,2-a]pyrazine-2(1H)-carboxylate;
4-Cyclopropyl-N—((S)-1-(4,4-difluorocyclohexyl)-2-oxo-2-((4-(((S)-2-oxo-4-(trifluoromethyl)imidazolidin-1-yl)methyl)pyridin-2-yl)amino)ethyl)-1,2,5-oxadiazole-3-carboxamide;
N—((S)-1-(4,4-Difluorocyclohexyl)-2-oxo-2-((4-(((S)-2-oxo-4-(trifluoromethyl)-imidazolidin-1-yl)methyl)pyridin-2-yl)amino)ethyl)-1-methyl-1H-pyrazole-5-carboxamide;
Benzyl ((S)-1-(4,4-difluorocyclohexyl)-2-oxo-2-((4-(((S)-2-oxo-4-(trifluoromethyl)-imidazolidin-1-yl)methyl)pyridin-2-yl)amino)ethyl)carbamate;
Tert-butyl 6-(((S)-1-(4,4-difluorocyclohexyl)-2-oxo-2-((4-(((S)-2-oxo-4-(trifluoromethyl)imidazolidin-1-yl)methyl)pyridin-2-yl)amino)ethyl)carbamoyl)-3,4-dihydropyrrolo[1,2-a]pyrazine-2(1H)-carboxylate;
N—((S)-1-(4,4-Difluorocyclohexyl)-2-oxo-2-((4-(((S)-2-oxo-4-(trifluoromethyl)-imidazolidin-1-yl)methyl)pyridin-2-yl)amino)ethyl)-1-isopropyl-1H-pyrazole-5-carboxamide;
N—((S)-1-(4,4-Difluorocyclohexyl)-2-oxo-2-((4-(((S)-2-oxo-4-(trifluoromethyl)-imidazolidin-1-yl)methyl)pyridin-2-yl)amino)ethyl)-3-ethylisoxazole-4-carboxamide;
1-Cyclopropyl-N—((S)-1-(4,4-difluorocyclohexyl)-2-oxo-2-((4-(((S)-2-oxo-4-(trifluoromethyl)imidazolidin-1-yl)methyl)pyridin-2-yl)amino)ethyl)-1H-pyrazole-5-carboxamide;
N—((S)-1,1-Dicyclopropyl-3-oxo-3-((4-(((S)-2-oxo-4-(trifluoromethyl)imidazolidin-1-yl)methyl)pyridin-2-yl)amino)propan-2-yl)-1-methyl-1H-pyrazole-5-carboxamide;
4-Cyclopropyl-N—((S)-1,1-dicyclopropyl-3-oxo-3-((4-(((S)-2-oxo-4-(trifluoromethyl)-imidazolidin-1-yl)methyl)pyridin-2-yl)amino)propan-2-yl)-1,2,5-oxadiazole-3-carboxamide;
N—((S)-1,1-Dicyclopropyl-3-oxo-3-((4-(((S)-2-oxo-4-(trifluoromethyl)imidazolidin-1-yl)methyl)pyridin-2-yl)amino)propan-2-yl)-1-isopropyl-1H-pyrazole-5-carboxamide;
N—((S)-1,1-Dicyclopropyl-3-oxo-3-((4-(((S)-2-oxo-4-(trifluoromethyl)imidazolidin-1-yl)methyl)pyridin-2-yl)amino)propan-2-yl)-4-methyl-1,2,5-oxadiazole-3-carboxamide;
Tert-butyl ((1-(4-(((S)-1-(4,4-difluorocyclohexyl)-2-oxo-2-((4-(((S)-2-oxo-4-(trifluoromethyl)imidazolidin-1-yl)methyl)pyridin-2-yl)amino)ethyl)carbamoyl)isoxazol-3-yl)cyclopropyl)methyl)carbamate;
3-Cyclopropyl-N—((S)-1-(4,4-difluorocyclohexyl)-2-oxo-2-((4-(((S)-2-oxo-4-(trifluoromethyl)imidazolidin-1-yl)methyl)pyridin-2-yl)amino)ethyl)isoxazole-4-carboxamide;
N—((S)-1-(4,4-Difluorocyclohexyl)-2-oxo-2-((4-(((S)-2-oxo-4-(trifluoromethyl)-imidazolidin-1-yl)methyl)pyridin-2-yl)amino)ethyl)-3-isopropylisoxazole-4-carboxamide;
3-(1-(Aminomethyl)cyclopropyl)-N—((S)-1-(4,4-difluorocyclohexyl)-2-oxo-2-((4-(((S)-2-oxo-4-(trifluoromethyl)imidazolidin-1-yl)methyl)pyridin-2-yl)amino)ethyl)isoxazole-4-carboxamide;
Tert-butyl ((S)-2-((4-((5-methyl-6-oxo-5,7-diazaspiro[3.4]octan-7-yl)methyl)pyridin-2-yl)amino)-1-((1r,4S)-4-methylcyclohexyl)-2-oxoethyl)carbamate;
Tert-butyl ((S)-2-((4-((4,4-dimethyl-2-oxoimidazolidin-1-yl)methyl)pyridin-2-yl)amino)-1-((1r,4S)-4-methylcyclohexyl)-2-oxoethyl)carbamate;
4-Cyclopropyl-N—((S)-2-((4-((4,4-dimethyl-2-oxoimidazolidin-1-yl)methyl)pyridin-2-yl)amino)-1-((1r,4S)-4-methylcyclohexyl)-2-oxoethyl)-1,2,5-oxadiazole-3-carboxamide;
Tert-butyl ((S)-1-((1r,4S)-4-methylcyclohexyl)-2-oxo-2-((4-((((2,2,2-trifluoroethyl)carbamoyl)oxy)methyl)pyridin-2-yl)amino)ethyl)carbamate;
Tert-butyl (S)-(1-(4,4-difluorocyclohexyl)-2-((4-((4,4-dimethyl-2,5-dioxoimidazolidin-1-yl)methyl)pyridin-2-yl)amino)-2-oxoethyl)carbamate;
Tert-butyl (S)-(1-(4,4-difluorocyclohexyl)-2-((4-((2,5-dioxoimidazolidin-1-yl)methyl)pyridin-2-yl)amino)-2-oxoethyl)carbamate;
Tert-butyl ((S)-2-((4-(((R)-5-methyl-2-oxoimidazolidin-1-yl)methyl)pyridin-2-yl)amino)-1-((1r,4S)-4-methylcyclohexyl)-2-oxoethyl)carbamate;
Tert-butyl ((S)-2-((4-(((R)-4-methyl-2-oxoimidazolidin-1-yl)methyl)pyridin-2-yl)amino)-1-((1r,4S)-4-methylcyclohexyl)-2-oxoethyl)carbamate;
(S)—N-(1,1-Dicyclopropyl-3-oxo-3-((4-(2-oxo-2-((3,3,3-trifluoropropyl)amino)ethyl)-pyridin-2-yl)amino)propan-2-yl)-1-methyl-1H-pyrazole-5-carboxamide;
(S)—N-(1,1-Dicyclopropyl-3-oxo-3-((4-((4,4,4-trifluorobutanamido)methyl)pyridin-2-yl)amino)propan-2-yl)-1-methyl-1H-pyrazole-5-carboxamide;
N—((S)-1,1-Dicyclopropyl-3-((4-(((R)-4-methyl-2-oxoimidazolidin-1-yl)methyl)pyridin-2-yl)amino)-3-oxopropan-2-yl)-1-methyl-1H-pyrazole-5-carboxamide;
1-Cyclopropyl-N—((S)-1,1-dicyclopropyl-3-((4-(((R)-4-methyl-2-oxoimidazolidin-1-yl)methyl)pyridin-2-yl)amino)-3-oxopropan-2-yl)-1H-pyrazole-5-carboxamide;
N-(1-(4,4-Difluorocyclohexyl)-2-((4-((1-methyl-6-oxo-1,6-dihydropyridin-2-yl)methyl)pyridin-2-yl)amino)-2-oxoethyl)-1-isopropyl-1H-pyrazole-5-carboxamide;
N-(1-(4,4-Difluorocyclohexyl)-2-((4-((1-methyl-6-oxo-1,6-dihydropyridin-2-yl)methyl)pyridin-2-yl)amino)-2-oxoethyl)-1-ethyl-1H-pyrazole-5-carboxamide;
N-(1-(4,4-Difluorocyclohexyl)-2-((4-((1-methyl-6-oxo-1,6-dihydropyridin-2-yl)methyl)pyridin-2-yl)amino)-2-oxoethyl)-1-methyl-1H-pyrazole-5-carboxamide;
N-((2S)-1,1-Dicyclopropyl-3-((4-(cyclopropyl(4,4,4-trifluorobutanamido)methyl)pyridin-2-yl)amino)-3-oxopropan-2-yl)-1-methyl-1H-pyrazole-5-carboxamide;
4-Cyclopropyl-N-((2S)-1,1-dicyclopropyl-3-((4-(cyclopropyl(4,4,4-trifluorobutanamido)methyl)pyridin-2-yl)amino)-3-oxopropan-2-yl)-1,2,5-oxadiazole-3-carboxamide;
N-((2S)-1,1-Dicyclopropyl-3-((4-(cyclopropyl(4,4,4-trifluorobutanamido)-methyl)pyridin-2-yl)amino)-3-oxopropan-2-yl)-1-isopropyl-1H-pyrazole-5-carboxamide;
N-((2S)-1,1-Dicyclopropyl-3-((4-(cyclopropyl(4,4,4-trifluorobutanamido)-methyl)pyridin-2-yl)amino)-3-oxopropan-2-yl)-3-ethylisoxazole-4-carboxamide;
Tert-butyl (S)-(1-(4,4-difluorocyclohexyl)-2-oxo-2-((4-((((2,2,2-trifluoroethyl)-carbamoyl)oxy)methyl)pyridin-2-yl)amino)ethyl)carbamate;
4-Cyclopropyl-N-(2-((4-(cyclopropyl(4,4,4-trifluorobutanamido)methyl)pyridin-2-yl)amino)-1-(4,4-difluorocyclohexyl)-2-oxoethyl)-1,2,5-oxadiazole-3-carboxamide;
N-(2-((4-(Cyclopropyl(4,4,4-trifluorobutanamido)methyl)pyridin-2-yl)amino)-1-(4,4-difluorocyclohexyl)-2-oxoethyl)-1-methyl-1H-pyrazole-5-carboxamide;
N-(2-((4-(Cyclopropyl(4,4,4-trifluorobutanamido)methyl)pyridin-2-yl)amino)-1-(4,4-difluorocyclohexyl)-2-oxoethyl)-1-isopropyl-1H-pyrazole-5-carboxamide;
N-(2-((4-(Cyclopropyl(4,4,4-trifluorobutanamido)methyl)pyridin-2-yl)amino)-1-(4,4-difluorocyclohexyl)-2-oxoethyl)-3-ethylisoxazole-4-carboxamide;
Tert-butyl ((S)-2-((5-fluoro-4-(((S)-2-oxo-4-(trifluoromethyl)imidazolidin-1-yl)methyl)pyridin-2-yl)amino)-1-((1r,4S)-4-methylcyclohexyl)-2-oxoethyl)carbamate;
Tert-butyl ((S)-2-((4-((1,4-dimethyl-1H-pyrazol-5-yl)methyl)pyridin-2-yl)amino)-1-((1r,4S)-4-methylcyclohexyl)-2-oxoethyl)carbamate;
N-(1-(4,4-Difluorocyclohexyl)-2-((4-((1-methyl-6-oxo-1,6-dihydropyridin-3-yl)methyl)pyridin-2-yl)amino)-2-oxoethyl)-1-methyl-1H-pyrazole-5-carboxamide;
N-(1-(4,4-Difluorocyclohexyl)-2-((4-((1,4-dimethyl-1H-pyrazol-5-yl)methyl)pyridin-2-yl)amino)-2-oxoethyl)-1-methyl-1H-pyrazole-5-carboxamide;
N-(1-(4,4-Difluorocyclohexyl)-2-((4-((1-methyl-2-oxo-1,2-dihydropyridin-4-yl)methyl)pyridin-2-yl)amino)-2-oxoethyl)-1-methyl-1H-pyrazole-5-carboxamide;
N—((S)-2-((4-((1,4-Dimethyl-1H-pyrazol-5-yl)methyl)pyridin-2-yl)amino)-1-((1r,4S)-4-methylcyclohexyl)-2-oxoethyl)-1-isopropyl-1H-pyrazole-5-carboxamide;
(1R,2S,5S)—N-((2S)-1,1-Dicyclopropyl-3-((4-(cyclopropyl(4,4,4-trifluorobutanamido)-methyl)pyridin-2-yl)amino)-3-oxopropan-2-yl)-3,6,6-trimethyl-3-azabicyclo[3.1.0]-hexane-2-carboxamide;
(5-Methylisoxazol-3-yl)methyl ((S)-1-(4,4-difluorocyclohexyl)-2-oxo-2-((4-(((S)-2-oxo-4-(trifluoromethyl)imidazolidin-1-yl)methyl)pyridin-2-yl)amino)ethyl)carbamate;
4-Cyclopropyl-N-((1S)-2-((4-(2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)-1,2,5-oxadiazole-3-carboxamide;
(1,5-Dimethyl-1H-pyrazol-3-yl)methyl ((S)-1-(4,4-difluorocyclohexyl)-2-oxo-2-((4-(((S)-2-oxo-4-(trifluoromethyl)imidazolidin-1-yl)methyl)pyridin-2-yl)amino)ethyl)carbamate;
4-Cyclopropyl-N—((S)-2-((5-fluoro-4-(((S)-2-oxo-4-(trifluoromethyl)imidazolidin-1-yl)methyl)pyridin-2-yl)amino)-1-((1r,4S)-4-methylcyclohexyl)-2-oxoethyl)-1,2,5-oxadiazole-3-carboxamide;
N—((S)-2-((5-Fluoro-4-(((S)-2-oxo-4-(trifluoromethyl)imidazolidin-1-yl)methyl)pyridin-2-yl)amino)-1-((1r,4S)-4-methylcyclohexyl)-2-oxoethyl)-4-methyl-1,2,5-oxadiazole-3-carboxamide;
N—((S)-2-((5-Fluoro-4-(((S)-2-oxo-4-(trifluoromethyl)imidazolidin-1-yl)methyl)pyridin-2-yl)amino)-1-((1r,4S)-4-methylcyclohexyl)-2-oxoethyl)-1-isopropyl-1H-pyrazole-5-carboxamide;
N—((S)-2-((5-Fluoro-4-(((S)-2-oxo-4-(trifluoromethyl)imidazolidin-1-yl)methyl)pyridin-2-yl)amino)-1-((1r,4S)-4-methylcyclohexyl)-2-oxoethyl)-1-methyl-1H-pyrazole-5-carboxamide;
Tert-butyl ((S)-2-((4-((S)-2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)carbamate;
Tert-butyl ((S)-2-((4-((R)-2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)carbamate;
1-Fluoro-N—((S)-1-((1r,4S)-4-methylcyclohexyl)-2-oxo-2-((4-(((S)-2-oxo-4-(trifluoromethyl)imidazolidin-1-yl)methyl)pyridin-2-yl)amino)ethyl)cyclopropane-1-carboxamide;
N—((S)-1-((1r,4S)-4-Methylcyclohexyl)-2-oxo-2-((4-(((S)-2-oxo-4-(trifluoromethyl)-imidazolidin-1-yl)methyl)pyridin-2-yl)amino)ethyl)-1-(4,4,4-trifluoro-3-hydroxybutyl)-1H-pyrazole-5-carboxamide;
(4-Methyl-1,2,5-oxadiazol-3-yl)methyl ((S)-1-(4,4-difluorocyclohexyl)-2-oxo-2-((4-(((S)-2-oxo-4-(trifluoromethyl)imidazolidin-1-yl)methyl)pyridin-2-yl)amino)ethyl);
Tert-butyl ((1S)-2-((4-(cyclopropyl(4,4,4-trifluorobutanamido)methyl)pyridin-2-yl)amino)-1-((1r,4S)-4-methylcyclohexyl)-2-oxoethyl)carbamate;
Tert-butyl ((1S)-2-((4-(cyclopropyl(4,4,4-trifluorobutanamido)methyl)pyridin-2-yl)amino)-1-(4,4-difluorocyclohexyl)-2-oxoethyl)carbamate;
N-((1S)-2-((4-(2-Methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)-4-methyl-1,2,5-oxadiazole-3-carboxamide;
N-((1S)-2-((4-(2-Methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)-1-methyl-1H-pyrazole-5-carboxamide;
3-Isopropyl-N-((1S)-2-((4-(2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)isoxazole-4-carboxamide;
1-Isopropyl-N-((1S)-2-((4-(2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)-1H-pyrazole-5-carboxamide;
Tert-butyl ((S)-2-((4-((3,5-dimethyl-1H-pyrazol-4-yl)methyl)pyridin-2-yl)amino)-1-((1r,4S)-4-methylcyclohexyl)-2-oxoethyl)carbamate;
Tert-butyl ((S)-1-((1r,4S)-4-methylcyclohexyl)-2-oxo-2-((5-(((S)-2-oxo-4-(trifluoromethyl)imidazolidin-1-yl)methyl)pyridin-3-yl)amino)ethyl)carbamate;
Tert-butyl ((R)-1-((1r,4R)-4-methylcyclohexyl)-2-oxo-2-((5-(((S)-2-oxo-4-(trifluoromethyl)imidazolidin-1-yl)methyl)pyridin-3-yl)amino)ethyl)carbamate;
Benzyl ((rac)-3-(tert-butoxy)-1-oxo-1-((4-(((S)-2-oxo-4-(trifluoromethyl)imidazolidin-1-yl)methyl)pyridin-2-yl)amino)propan-2-yl)carbamate;
N-(3-(Tert-butoxy)-1-oxo-1-((4-(((S)-2-oxo-4-(trifluoromethyl)imidazolidin-1-yl)methyl)pyridin-2-yl)amino)propan-2-yl)-4-cyclopropyl-1,2,5-oxadiazole-3-carboxamide;
N-(3-(Tert-butoxy)-1-oxo-1-((4-(((S)-2-oxo-4-(trifluoromethyl)imidazolidin-1-yl)methyl)pyridin-2-yl)amino)propan-2-yl)-1-methyl-1H-pyrazole-5-carboxamide;
4-Cyclopropyl-N—((S)-1-((1r,4S)-4-methylcyclohexyl)-2-oxo-2-((5-(((S)-2-oxo-4-(trifluoromethyl)imidazolidin-1-yl)methyl)pyridin-3-yl)amino)ethyl)-1,2,5-oxadiazole-3-carboxamide;
4-Cyclopropyl-N—((R)-1-((1r,4R)-4-methylcyclohexyl)-2-oxo-2-((5-(((S)-2-oxo-4-(trifluoromethyl)imidazolidin-1-yl)methyl)pyridin-3-yl)amino)ethyl)-1,2,5-oxadiazole-3-carboxamide;
4-Ethyl-N-((1S)-2-((4-(2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)-1,2,5-oxadiazole-3-carboxamide;
N-((1S)-2-((4-(2-Methoxy-1-((RS)-2-oxo-4-(trifluoromethyl)imidazolidin-1-yl)ethyl)pyridin-2-yl)amino)-1-((1r,4S)-4-methylcyclohexyl)-2-oxoethyl)-2-methyl-4-(trifluoromethyl)pyrimidine-5-carboxamide;
N-((1S)-2-((4-(2-Methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)-5-methylthiazole-4-carboxamide;
5-Cyano-N-((1S)-2-((4-(2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)thiazole-4-carboxamide;
N-((1S)-2-((4-(2-Methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)-1-methyl-1H-tetrazole-5-carboxamide;
6-Bromo-N-((1S)-2-((4-(2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)-3-methylpicolinamide;
2-Chloro-N-((1S)-2-((4-(2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)-5-methylpyrimidine-4-carboxamide;
4-Cyclopropyl-N-((1S)-2-((4-(cyclopropyl(4,4,4-trifluorobutanamido)methyl)pyridin-2-yl)amino)-1-(4,4-difluorocyclohexyl)-2-oxoethyl)-1,2,5-oxadiazole-3-carboxamide;
(S)-2-(3-Benzylureido)-2-((1r,4S)-4-methylcyclohexyl)-N-(4-(((S)-2-oxo-4-(trifluoromethyl)imidazolidin-1-yl)methyl)pyridin-2-yl)acetamide;
(S)-2-(3-Benzylureido)-2-(4,4-difluorocyclohexyl)-N-(4-(((S)-2-oxo-4-(trifluoromethyl)imidazolidin-1-yl)methyl)pyridin-2-yl)acetamide;
Benzyl ((2R,3R)-3-(tert-butoxy)-1-oxo-1-((4-(((S)-2-oxo-4-(trifluoromethyl)imidazolidin-1-yl)methyl)pyridin-2-yl)amino)butan-2-yl)carbamate;
Benzyl ((2S,3R)-3-(tert-butoxy)-1-oxo-1-((4-(((S)-2-oxo-4-(trifluoromethyl)imidazolidin-1-yl)methyl)pyridin-2-yl)amino)butan-2-yl)carbamate;
N-((2S,3R)-3-(Tert-butoxy)-1-oxo-1-((4-(((S)-2-oxo-4-(trifluoromethyl)imidazolidin-1-yl)methyl)pyridin-2-yl)amino)butan-2-yl)-4-cyclopropyl-1,2,5-oxadiazole-3-carboxamide;
N-((2S,3R)-3-(Tert-butoxy)-1-oxo-1-((4-(((S)-2-oxo-4-(trifluoromethyl)imidazolidin-1-yl)methyl)pyridin-2-yl)amino)butan-2-yl)-1-methyl-1H-pyrazole-5-carboxamide;
Tert-butyl ((S)-1,1-dicyclopropyl-3-oxo-3-((4-(((S)-2-oxo-4-(trifluoromethyl)-imidazolidin-1-yl)methyl)pyridin-2-yl)amino)propan-2-yl)carbamate;
N-((1S)-2-((4-(Cyclopropyl(4,4,4-trifluorobutanamido)methyl)pyridin-2-yl)amino)-1-(4,4-difluorocyclohexyl)-2-oxoethyl)oxazole-2-carboxamide;
N-((1S)-2-((4-(Cyclopropyl(4,4,4-trifluorobutanamido)methyl)pyridin-2-yl)amino)-1-((1r,4S)-4-methylcyclohexyl)-2-oxoethyl)-1-isopropyl-1H-pyrazole-5-carboxamide;
N—((S)-2-((4-((R)-Cyclopropyl(4,4,4-trifluorobutanamido)methyl)pyridin-2-yl)amino)-1-(4,4-difluorocyclohexyl)-2-oxoethyl)-1-methyl-1H-pyrazole-5-carboxamide;
N-((1S)-2-((4-(Cyclopropyl(4,4,4-trifluorobutanamido)methyl)pyridin-2-yl)amino)-1-(4,4-difluorocyclohexyl)-2-oxoethyl)-1-ethyl-1H-pyrazole-5-carboxamide;
N-((1S)-2-((4-(Cyclopropyl(4,4,4-trifluorobutanamido)methyl)pyridin-2-yl)amino)-1-(4,4-difluorocyclohexyl)-2-oxoethyl)oxazole-4-carboxamide;
N-((1S)-2-((4-(Cyclopropyl(4,4,4-trifluorobutanamido)methyl)pyridin-2-yl)amino)-1-(4,4-difluorocyclohexyl)-2-oxoethyl)-1-isopropyl-1H-pyrazole-5-carboxamide;
N-((1S)-2-((4-(Cyclopropyl(4,4,4-trifluorobutanamido)methyl)pyridin-2-yl)amino)-1-(4,4-difluorocyclohexyl)-2-oxoethyl)-1-methyl-1H-imidazole-2-carboxamide;
N-((1S)-2-((4-(2-Methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)-1-methyl-1H-imidazole-2-carboxamide;
(2S)-2-(2-(3,5-Dimethylisoxazol-4-yl)acetamido)-N-(4-(2-methoxy-1-((S)-2-oxo-4-(trifluoromethyl)imidazolidin-1-yl)ethyl)pyridin-2-yl)-2-((1r,4S)-4-methylcyclohexyl)acetamide;
(2S)—N-(4-(2-Methoxy-1-((S)-2-oxo-4-(trifluoromethyl)imidazolidin-1-yl)ethyl)pyridin-2-yl)-2-(2-(5-methyl-1H-pyrazol-3-yl)acetamido)-2-((1r,4S)-4-methylcyclohexyl)-acetamide;
1-Isopropyl-N-((1S)-2-((4-(2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)-1H-imidazole-2-carboxamide;
N-((2S,3S)-3-(Tert-butoxy)-1-oxo-1-((4-(((S)-2-oxo-4-(trifluoromethyl)imidazolidin-1-yl)methyl)pyridin-2-yl)amino)butan-2-yl)-4-cyclopropyl-1,2,5-oxadiazole-3-carboxamide;
N-((1S)-2-((4-(Cyclopropyl(4,4,4-trifluorobutanamido)methyl)pyridin-2-yl)amino)-1-(4,4-difluorocyclohexyl)-2-oxoethyl)-1-isopropyl-1H-imidazole-2-carboxamide;
1-Methyl-N-((1S)-1-((1r,4S)-4-methylcyclohexyl)-2-oxo-2-((4-((2-oxopyrrolidin-3-yl)oxy)pyridin-2-yl)amino)ethyl)-1H-pyrazole-5-carboxamide;
1-Isopropyl-N-((1S)-1-((1r,4S)-4-methylcyclohexyl)-2-oxo-2-((4-((2-oxopyrrolidin-3-yl)oxy)pyridin-2-yl)amino)ethyl)-1H-pyrazole-5-carboxamide;
N—((S)-2-((4-(2-Methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)-4-methylisoxazole-3-carboxamide;
4-Cyclopropyl-N—((S)-2-((4-(2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)isoxazole-3-carboxamide;
N—((S)-2-((4-(2-Methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)-4-methyloxazole-5-carboxamide;
(S)—N-(4-(2-Methoxy-1-((S)-2-oxo-4-(trifluoromethyl)imidazolidin-1-yl)ethyl)pyridin-2-yl)-2-((1r,4S)-4-methylcyclohexyl)-2-(2-(3-methylisoxazol-5-yl)acetamido)acetamide;
1-Ethyl-N—((S)-2-((4-(2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)-1H-pyrazole-5-carboxamide;
3-Ethyl-N—((S)-2-((4-(2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)isoxazole-4-carboxamide;
3-Cyclopropyl-N—((S)-2-((4-(2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)isoxazole-4-carboxamide;
N-((2S,3R)-3-(Tert-butoxy)-1-oxo-1-((4-(((S)-2-oxo-4-(trifluoromethyl)imidazolidin-1-yl)methyl)pyridin-2-yl)amino)butan-2-yl)-1-isopropyl-1H-pyrazole-5-carboxamide;
N-((2S,3R)-3-(Tert-butoxy)-1-oxo-1-((4-(((S)-2-oxo-4-(trifluoromethyl)imidazolidin-1-yl)methyl)pyridin-2-yl)amino)butan-2-yl)-4-methyl-1,2,5-oxadiazole-3-carboxamide;
N-((2S,3R)-3-(Tert-butoxy)-1-oxo-1-((4-(((S)-2-oxo-4-(trifluoromethyl)imidazolidin-1-yl)methyl)pyridin-2-yl)amino)butan-2-yl)-4-ethyl-1,2,5-oxadiazole-3-carboxamide;
N-((2S,3R)-3-(Tert-butoxy)-1-oxo-1-((4-(((S)-2-oxo-4-(trifluoromethyl)imidazolidin-1-yl)methyl)pyridin-2-yl)amino)butan-2-yl)-3-isopropylisoxazole-4-carboxamide;
Tert-butyl ((1S)-1-(4,4-difluorocyclohexyl)-2-((4-(2-methoxy-1-(4,4,4-trifluorobutanamido)ethyl)pyridin-2-yl)amino)-2-oxoethyl)carbamate;
4-Cyclopropyl-N-((1S)-1-(4,4-difluorocyclohexyl)-2-((4-(2-methoxy-1-(4,4,4-trifluorobutanamido)ethyl)pyridin-2-yl)amino)-2-oxoethyl)-1,2,5-oxadiazole-3-carboxamide;
N-((1S)-1-(4,4-Difluorocyclohexyl)-2-((4-(2-methoxy-1-(4,4,4-trifluorobutanamido)ethyl)pyridin-2-yl)amino)-2-oxoethyl)-1-methyl-1H-pyrazole-5-carboxamide;
N-((1S)-1-(4,4-Difluorocyclohexyl)-2-((4-(2-methoxy-1-(4,4,4-trifluorobutanamido)ethyl)pyridin-2-yl)amino)-2-oxoethyl)-1-isopropyl-1H-pyrazole-5-carboxamide;
N-((1S)-1-(4,4-Difluorocyclohexyl)-2-((4-(2-methoxy-1-(4,4,4-trifluorobutanamido)ethyl)pyridin-2-yl)amino)-2-oxoethyl)-1-ethyl-1H-pyrazole-5-carboxamide;
1-Cyclopropyl-N-((1S)-1-(4,4-difluorocyclohexyl)-2-((4-(2-methoxy-1-(4,4,4-trifluorobutanamido)ethyl)pyridin-2-yl)amino)-2-oxoethyl)-1H-pyrazole-5-carboxamide;
N-((1S)-1-(4,4-Difluorocyclohexyl)-2-((4-(2-methoxy-1-(4,4,4-trifluorobutanamido)ethyl)pyridin-2-yl)amino)-2-oxoethyl)-1-methyl-1H-imidazole-2-carboxamide;
N-((1S)-1-(4,4-Difluorocyclohexyl)-2-((4-(2-methoxy-1-(4,4,4-trifluorobutanamido)ethyl)pyridin-2-yl)amino)-2-oxoethyl)-1-isopropyl-1H-imidazole-2-carboxamide;
1-(Cyclopropylmethyl)-N-((1S)-1-(4,4-difluorocyclohexyl)-2-((4-(2-methoxy-1-(4,4,4-trifluorobutanamido)ethyl)pyridin-2-yl)amino)-2-oxoethyl)-1H-pyrazole-5-carboxamide;
2,2-Difluoro-N—((S)-2-((4-((S)-2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)-2-(p-tolyl)acetamide;
2,2-Difluoro-N-((1S)-2-((4-(2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)-2-phenylacetamide;
Tert-butyl ((1S)-1-((1r,4S)-4-methylcyclohexyl)-2-oxo-2-((4-(((S)-2-oxo-4-(trifluoromethyl)-imidazolidin-1-yl)methyl-d)pyridin-2-yl)amino)ethyl)carbamate;
4-Cyclopropyl-N—((S)-1-((1r,4S)-4-methylcyclohexyl)-2-oxo-2-((4-(((3R,5S)-2-oxo-5-(trifluoromethyl)pyrrolidin-3-yl)methyl)pyridin-2-yl)amino)ethyl)-1,2,5-oxadiazole-3-carboxamide;
1-Ethyl-N—((S)-1-((1r,4S)-4-methylcyclohexyl)-2-oxo-2-((4-(((3R,5S)-2-oxo-5-(trifluoromethyl)pyrrolidin-3-yl)methyl)pyridin-2-yl)amino)ethyl)-1H-pyrazole-5-carboxamide;
1-Isopropyl-N—((S)-1-((1r,4S)-4-methylcyclohexyl)-2-oxo-2-((4-(((3R,5S)-2-oxo-5-(trifluoromethyl)pyrrolidin-3-yl)methyl)pyridin-2-yl)amino)ethyl)-1H-pyrazole-5-carboxamide;
1-Ethyl-N—((S)-1-((1r,4S)-4-methylcyclohexyl)-2-oxo-2-((4-(((3S,5S)-2-oxo-5-(trifluoromethyl)pyrrolidin-3-yl)methyl)pyridin-2-yl)amino)ethyl)-1H-pyrazole-5-carboxamide;
1-Isopropyl-N—((S)-1-((1r,4S)-4-methylcyclohexyl)-2-oxo-2-((4-(((3S,5S)-2-oxo-5-(trifluoromethyl)pyrrolidin-3-yl)methyl)pyridin-2-yl)amino)ethyl)-1H-pyrazole-5-carboxamide;
N-((1S)-1-(4,4-difluorocyclohexyl)-2-oxo-2-((4-(3,3,3-trifluoro-1-(4,4,4-trifluorobutanamido)propyl)pyridin-2-yl)amino)ethyl)-4-ethylisoxazole-5-carboxamide;
N—((S)-1-(4,4-difluorocyclohexyl)-2-oxo-2-((4-((R)-1-((S)-2-oxo-4-(trifluoromethyl)imidazolidin-1-yl)butyl)pyridin-2-yl)amino)ethyl)-4-ethylisoxazole-3-carboxamide;
N—((S)-1-(4,4-difluorocyclohexyl)-2-((4-((R)-2-methyl-1-((S)-2-oxo-4-(trifluoromethyl)imidazolidin-1-yl)propyl)pyridin-2-yl)amino)-2-oxoethyl)-4-ethylisoxazole-3-carboxamide;
N-((1S)-2-((4-(cyclopropyl(3,3,3-trifluoropropanamido)methyl)pyridin-2-yl)amino)-1-(4,4-difluorocyclohexyl)-2-oxoethyl)-1-isopropyl-1H-pyrazole-5-carboxamide;
N-((1S)-2-((4-(cyclopropyl(4,4,4-trifluorobutanamido)methyl)pyridin-2-yl)amino)-1-(4,4-difluorocyclohexyl)-2-oxoethyl)-3-ethylisoxazole-4-carboxamide;
4-Cyclopropyl-N—((S)-2-((4-(cyclopropyl((S)-2-oxo-4-(trifluoromethyl)imidazolidin-1-yl)methyl)pyridin-2-yl)amino)-1-(4,4-difluorocyclohexyl)-2-oxoethyl)-1,2,5-oxadiazole-3-carboxamide;
N-((1S)-2-((4-(cyclopropyl((S)-2-oxo-4-(trifluoromethyl)imidazolidin-1-yl)methyl)pyridin-2-yl)amino)-1-(4,4-difluoro cyclohexyl)-2-oxoethyl)-4-methyl-1,2,5-oxadiazole-3-carboxamide;
N-((1S)-2-((4-((cyclopropyl((S)-2-oxo-4-(trifluoromethyl)imidazolidin-1-yl)methyl)pyridin-2-yl)amino)-1-(4,4-difluoro cyclohexyl)-2-oxoethyl)-1-ethyl-1H-pyrazole-5-carboxamide;
N-((1S)-2-((4-(cyclopropyl((S)-2-oxo-4-(trifluoromethyl) imidazolidin-1-yl)methyl) pyridin-2-yl)amino)-1-(4,4-difluorocyclohexyl)-2-oxoethyl)-1-methyl-1H-pyrazole-5-carboxamide;
N—((S)-1-(4,4-difluorocyclohexyl)-2-oxo-2-((4-((R)-1-(4,4,4-trifluorobutanamido)ethyl)pyridin-2-yl)amino)ethyl)-1-isopropyl-1H-pyrazole-5-carboxamide;
4-Cyclopropyl-N—((S)-1-(4,4-difluorocyclohexyl)-2-((4-((R)-1-(4-methyl-2-oxo-2,3-dihydro-1H-imidazol-1-yl)ethyl)pyridin-2-yl)amino)-2-oxoethyl)-1,2,5-oxadiazole-3-carboxamide;
N-((1S)-1-(4,4-Difluorocyclohexyl)-2-oxo-2-((4-(1-((S)-2-oxo-4-(trifluoromethyl)imidazolidin-1-yl)ethyl)pyridin-2-yl)amino)ethyl)-4-methyl-1,2,5-oxadiazole-3-carboxamide;
4-cyclopropyl-N—((S)-1-(4,4-difluorocyclohexyl)-2-oxo-2-((4-(1-((S)-2-oxo-4-(trifluoromethyl)imidazolidin-1-yl)ethyl)pyridin-2-yl)amino) ethyl)-1,2,5-oxadiazole-3-carboxamide;
N-((1S)-1-(4,4-difluorocyclohexyl)-2-oxo-2-((4-(1-((S)-2-oxo-4-(trifluoromethyl) imidazolidin-1-yl)ethyl) pyridin-2-yl)amino) ethyl)-1-methyl-1H-pyrazole-5-carboxamide;
N—((S)-1-(4,4-difluorocyclohexyl)-2-oxo-2-((4-(2-((S)-2-oxo-4-(trifluoromethyl)imidazolidin-1-yl)propan-2-yl)pyridin-2-yl)amino)ethyl)-1-methyl-1H-pyrazole-5-carboxamide;
N—((S)-1-(4,4-difluorocyclohexyl)-2-oxo-2-((4-(2-((S)-2-oxo-4-(trifluoromethyl)imidazolidin-1-yl)propan-2-yl)pyridin-2-yl)amino)ethyl)-3-ethylisoxazole-4-carboxamide;
N—((S)-1-(4,4-difluorocyclohexyl)-2-oxo-2-((4-(2-((S)-2-oxo-4-(trifluoromethyl)imidazolidin-1-yl)propan-2-yl)pyridin-2-yl)amino)ethyl)-1-ethyl-1H-pyrazole-5-carboxamide;
N—((S)-1-(4,4-difluorocyclohexyl)-2-oxo-2-((4-(2-((S)-2-oxo-4-(trifluoromethyl)imidazolidin-1-yl)propan-2-yl)pyridin-2-yl)amino)ethyl)-1-isopropyl-1H-pyrazole-5-carboxamide;
N—((S)-2-((4-((S)-1-(5,5-difluoro-2-oxotetrahydropyrimidin-1(2H)-yl)-2-methoxyethyl)pyridin-2-yl)amino)-1-(4,4-difluorocyclohexyl)-2-oxoethyl)-5-ethylisoxazole-4-carboxamide;
N—((S)-2-((4-((S)-1-(5,5-difluoro-2-oxotetrahydropyrimidin-1(2H)-yl)-2-methoxyethyl)pyridin-2-yl)amino)-1-(4,4-difluorocyclohexyl)-2-oxoethyl)-4-ethylisoxazole-3-carboxamide;
N—((S)-2-((4-((S)-1-(5,5-difluoro-2-oxotetrahydropyrimidin-1(2H)-yl)-2-methoxyethyl)pyridin-2-yl)amino)-1-(4,4-difluorocyclohexyl)-2-oxoethyl)-3-ethylisoxazole-4-carboxamide;
N—((S)-2-((4-((S)-1-(5,5-difluoro-2-oxotetrahydropyrimidin-1(2H)-yl)-2-methoxyethyl)pyridin-2-yl)amino)-1-(4,4-difluorocyclohexyl)-2-oxoethyl)-1-ethyl-1H-pyrazole-5-carboxamide;
N—((S)-2-((4-((S)-1-(5,5-difluoro-2-oxotetrahydropyrimidin-1(2H)-yl)-2-methoxyethyl)pyridin-2-yl)amino)-1-(4,4-difluorocyclohexyl)-2-oxoethyl)-1-methyl-1H-pyrazole-5-carboxamide;
N—((S)-2-((4-((S)-1-(5,5-difluoro-2-oxotetrahydropyrimidin-1(2H)-yl)-2-methoxyethyl)pyridin-2-yl)amino)-1-(4,4-difluorocyclohexyl)-2-oxoethyl)-1-isopropyl-1H-pyrazole-5-carboxamide;
N—((S)-2-((4-((S)-1-(5,5-difluoro-2-oxotetrahydropyrimidin-1(2H)-yl)-2-methoxyethyl)pyridin-2-yl)amino)-1-(4,4-difluorocyclohexyl)-2-oxoethyl)-4-ethyl-1,2,5-oxadiazole-3-carboxamide;
N—((S)-2-((4-((S)-1-(5,5-difluoro-2-oxotetrahydropyrimidin-1(2H)-yl)-2-methoxyethyl)pyridin-2-yl)amino)-1-(4,4-difluorocyclohexyl)-2-oxoethyl)-4-methyl-1,2,5-oxadiazole-3-carboxamide;
4-Cyclopropyl-N—((S)-1-(4,4-difluorocyclohexyl)-2-((4-(2-methoxy-1-((3S,5S)-2-oxo-5-(trifluoromethyl)pyrrolidin-3-yl)ethyl)pyridin-2-yl)amino)-2-oxoethyl)-1,2,5-oxadiazole-3-carboxamide;
4-Cyclopropyl-N-((1S)-1-(4,4-difluorocyclohexyl)-2-((4-(2-methoxy-1-((3R,5S)-2-oxo-5-(trifluoromethyl)pyrrolidin-3-yl)ethyl)pyridin-2-yl)amino)-2-oxoethyl)-1,2,5-oxadiazole-3-carboxamide;
4-Cyclopropyl-N-((1S)-1-(4,4-difluorocyclohexyl)-2-((4-(2-methoxy-1-((3R,5S)-2-oxo-5-(trifluoromethyl)pyrrolidin-3-yl)ethyl)pyridin-2-yl)amino)-2-oxoethyl)-1,2,5-oxadiazole-3-carboxamide;
N—((S)-1-(4,4-Difluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-4-ethyl-1,2,5-oxadiazole-3-carboxamide;
N—((S)-1-(4,4-difluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-4-isopropylisoxazole-3-carboxamide;
N—((S)-1-(4,4-difluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-1-(ethyl-d5)-1H-pyrazole-5-carboxamide;
N—((S)-1-(4,4-difluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-1-(ethyl-d5)-1H-pyrazole-3-carboxamide;
4-cyclobutyl-N—((S)-1-(4,4-difluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)isoxazole-3-carboxamide;
N—((S)-1-(4,4-difluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-4-(2,2-difluoroethoxy)isoxazole-3-carboxamide;
N—((S)-1-(4,4-difluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-4-(2,2,2-trifluoroethoxy)-1,2,5-oxadiazole-3-carboxamide;
N—((S)-1-(4,4-difluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-3-methylisoxazole-4-carboxamide;
N—((S)-1-(4,4-difluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-4-isopropyl-1,2,5-oxadiazole-3-carboxamide;
N—((S)-1-(4,4-difluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-4-methylisoxazole-3-carboxamide;
N—((S)-1-(4,4-difluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-3-isopropylisoxazole-4-carboxamide;
N—((S)-1-(4,4-difluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-3-ethylisoxazole-4-carboxamide;
N—((S)-1-(4,4-difluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-4-methyl-1,2,5-oxadiazole-3-carboxamide;
4-cyclopropyl-N—((S)-1-(4,4-difluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-1,2,5-oxadiazole-3-carboxamide;
N—((S)-1-(4,4-difluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-1-ethyl-1H-pyrazole-5-carboxamide;
N—((S)-1-(4,4-difluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-1-methyl-1H-pyrazole;
N—((S)-1-(4,4-difluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-1-isopropyl-1H-pyrazole-5-carboxamide;
(S)-2-(4,4-difluorocyclohexyl)-N-(4-((S)-2-methoxy-1-((S)-2-oxo-4-(trifluoromethyl)imidazolidin-1-yl)ethyl)pyridin-2-yl)-2-(2-(6-methoxypyridin-3-yl)acetamido)acetamide;
2-(3-cyanophenyl)-N—((S)-1-(4,4-difluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)propan amide;
(S)-2-(2-(3-cyanophenyl)acetamido)-2-(4,4-difluorocyclohexyl)-N-(4-((S)-2-methoxy-1-((S)-2-oxo-4-(trifluoromethyl)imidazolidin-1-yl)ethyl)pyridin-2-yl)acetamide;
N-((1S)-1-(4,4-difluorocyclohexyl)-2-oxo-2-((4-(1-(4,4,4-trifluorobutanamido)propyl)pyridin-2-yl)amino)ethyl)-4-ethyl-1,2,5-oxadiazole-3-carboxamide;
N—((S)-1-(4,4-difluorocyclohexyl)-2-oxo-2-((4-((R)-1-((S)-2-oxo-4-(trifluoromethyl)imidazolidin-1-yl)propyl)pyridin-2-yl)amino)ethyl)-4-ethylisoxazole-3-carboxamide;
N—((S)-1-(4,4-difluorocyclohexyl)-2-oxo-2-((4-((R)-1-((S)-2-oxo-4-(trifluoromethyl)imidazolidin-1-yl)propyl)pyridin-2-yl)amino)ethyl)-4-ethyl-1,2,5-oxadiazole-3-carboxamide;
N—((S)-1-(4,4-difluorocyclohexyl)-2-oxo-2-((4-((R)-1-((S)-2-oxo-4-(trifluoromethyl)imidazolidin-1-yl)propyl)pyridin-2-yl)amino)ethyl)-3-ethylisoxazole-4-carboxamide;
N—((S)-1-(4,4-difluorocyclohexyl)-2-oxo-2-((4-((R)-1-((S)-2-oxo-4-(trifluoromethyl)imidazolidin-1-yl)propyl)pyridin-2-yl)amino)ethyl)-4-methyl-1,2,5-oxadiazole-3-carboxamide;
4-cyclopropyl-N—((S)-1-(4,4-difluorocyclohexyl)-2-oxo-2-((4-((R)-1-((S)-2-oxo-4-(trifluoromethyl)imidazolidin-1-yl)propyl)pyridin-2-yl)amino)ethyl)-1,2,5-oxadiazole-3-carboxamide;
N—((S)-1-(4,4-difluorocyclohexyl)-2-oxo-2-((4-((R)-1-((S)-2-oxo-4-(trifluoromethyl)imidazolidin-1-yl)propyl)pyridin-2-yl)amino)ethyl)-1-ethyl-1H-pyrazole-5-carboxamide;
N—((S)-1-(4,4-difluorocyclohexyl)-2-oxo-2-((4-((R)-1-((S)-2-oxo-4-(trifluoromethyl)imidazolidin-1-yl)propyl)pyridin-2-yl)amino)ethyl)-1-methyl-1H-pyrazole-5-carboxamide;
N—((S)-1-(4,4-difluorocyclohexyl)-2-oxo-2-((4-((R)-1-((S)-2-oxo-4-(trifluoromethyl)imidazolidin-1-yl)propyl)pyridin-2-yl)amino)ethyl)-1-isopropyl-1H-pyrazole-5-carboxamide;
N-((1S)-1-(4,4-difluorocyclohexyl)-2-oxo-2-((4-((2-oxo-5-(trifluoromethyl)-2,5-dihydro-1H-pyrrol-3-yl)methyl)pyridin-2-yl)amino)ethyl)-3-ethylisoxazole-4-carboxamide;
N—((S)-1-(4,4-difluorocyclohexyl)-2-oxo-2-((4-(((3S,5S)-2-oxo-5-(trifluoromethyl)pyrrolidin-3-yl)methyl)pyridin-2-yl)amino)ethyl)-4-ethyloxazole-5-carboxamide;
N—((S)-1-(4,4-difluorocyclohexyl)-2-oxo-2-((4-(((3S,5S)-2-oxo-5-(trifluoromethyl)pyrrolidin-3-yl)methyl)pyridin-2-yl)amino)ethyl)-5-ethylisoxazole-4-carboxamide;
N—((S)-1-(4,4-difluorocyclohexyl)-2-oxo-2-((4-(((3S,5S)-2-oxo-5-(trifluoromethyl)pyrrolidin-3-yl)methyl)pyridin-2-yl)amino)ethyl)-5-isopropylisoxazole-4-carboxamide;
N—((S)-1-(4,4-difluorocyclohexyl)-2-oxo-2-((4-(((3S,5S)-2-oxo-5-(trifluoromethyl)pyrrolidin-3-yl)methyl)pyridin-2-yl)amino)ethyl)-4-ethyl-1,2,5-oxadiazole-3-carboxamide;
N—((S)-1-(4,4-difluorocyclohexyl)-2-oxo-2-((4-(((3S,5S)-2-oxo-5-(trifluoromethyl)pyrrolidin-3-yl)methyl)pyridin-2-yl)amino)ethyl)-4-methyl-1,2,5-oxadiazole-3-carboxamide;
4-cyclopropyl-N—((S)-1-(4,4-difluorocyclohexyl)-2-oxo-2-((4-(((3S,5S)-2-oxo-5-(trifluoromethyl)pyrrolidin-3-yl)methyl)pyridin-2-yl)amino)ethyl)-1,2,5-oxadiazole-3-carboxamide;
N—((S)-1-(4,4-difluorocyclohexyl)-2-oxo-2-((4-(((3S,5S)-2-oxo-5-(trifluoromethyl)pyrrolidin-3-yl)methyl)pyridin-2-yl)amino)ethyl)-1-ethyl-1H-pyrazole-5-carboxamide;
N—((S)-1-(4,4-difluorocyclohexyl)-2-oxo-2-((4-(((3S,5S)-2-oxo-5-(trifluoromethyl)pyrrolidin-3-yl)methyl)pyridin-2-yl)amino)ethyl)-1-methyl-1H-pyrazole-5-carboxamide;
N—((S)-1-(4,4-difluorocyclohexyl)-2-oxo-2-((4-(((3S,5S)-2-oxo-5-(trifluoromethyl)pyrrolidin-3-yl)methyl)pyridin-2-yl)amino)ethyl)-1-isopropyl-1H-pyrazole-5-carboxamide;
N—((S)-1,1-Dicyclopropyl-3-((4-((S)-2-methoxy-1-((S)-2-oxo-4-(trifluoromethyl)imidazolidin-1-yl)ethyl)pyridin-2-yl)amino)-3-oxopropan-2-yl)-4-methyl-1,2,5-oxadiazole-3-carboxamide;
4-cyclopropyl-N—((S)-1,1-dicyclopropyl-3-((4-((S)-2-methoxy-1-((S)-2-oxo-4-(trifluoromethyl)imidazolidin-1-yl)ethyl)pyridin-2-yl)amino)-3-oxopropan-2-yl)-1,2,5-oxadiazole-3-carboxamide;
N—((S)-1,1-dicyclopropyl-3-((4-((S)-2-methoxy-1-((S)-2-oxo-4-(trifluoromethyl)imidazolidin-1-yl)ethyl)pyridin-2-yl)amino)-3-oxopropan-2-yl)-1-methyl-1H-pyrazole-5-carboxamide;
N—((S)-1,1-dicyclopropyl-3-((4-((S)-2-methoxy-1-((S)-2-oxo-4-(trifluoromethyl) imidazolidin-1-yl)ethyl) pyridin-2-yl)amino)-3-oxopropan-2-yl)-1-isopropyl-1H-pyrazole-5-carboxamide;
N—((S)-1,1-dicyclopropyl-3-oxo-3-((4-(((3S,5S)-2-oxo-5-(trifluoromethyl)pyrrolidin-3-yl)methyl)pyridin-2-yl)amino)propan-2-yl)-1-ethyl-1H-pyrazole-5-carboxamide;
4-Cyclopropyl-N-((1S)-1-((1r,4S)-4-methylcyclohexyl)-2-oxo-2-((4-(((S)-2-oxo-4-(trifluoromethyl)imidazolidin-1-yl)methyl-d)pyridin-2-yl)amino)ethyl)-1,2,5-oxadiazole-3-carboxamide;
1-Methyl-N-((1S)-1-((1r,4S)-4-methylcyclohexyl)-2-oxo-2-((4-(((S)-2-oxo-4-(trifluoromethyl)imidazolidin-1-yl)methyl-d)pyridin-2-yl)amino)ethyl)-1H-pyrazole-5-carboxamide;
4-Cyclopropyl-N-((1S)-2-((4-(2-hydroxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)-1,2,5-oxadiazole-3-carboxamide;
4-Cyclopropyl-N-((1S)-2-((4-(cyclopropyl(4,4,4-trifluorobutanamido)methyl)pyridin-2-yl)amino)-1-((1r,4S)-4-methylcyclohexyl)-2-oxoethyl)-1,2,5-oxadiazole-3-carboxamide;
N-((1S)-2-((4-(cyclopropyl(4,4,4-trifluorobutanamido)methyl)pyridin-2-yl)amino)-1-((1r,4S)-4-methylcyclohexyl)-2-oxoethyl)-1-methyl-1H-pyrazole-5-carboxamide;
4-methyl-N—((S)-1-((1r,4S)-4-methylcyclohexyl)-2-oxo-2-((4-((R)-1-((S)-2-oxo-4-(trifluoromethyl)imidazolidin-1-yl)ethyl)pyridin-2-yl)amino)ethyl)-1,2,5-oxadiazole-3-carboxamide;
4-cyclopropyl-N—((S)-1-((1r,4S)-4-methylcyclohexyl)-2-oxo-2-((4-((R)-1-((S)-2-oxo-4-(trifluoromethyl)imidazolidin-1-yl)ethyl)pyridin-2-yl)amino)ethyl)-1,2,5-oxadiazole-3-carboxamide;
1-ethyl-N—((S)-1-((1r,4S)-4-methylcyclohexyl)-2-oxo-2-((4-((R)-1-((S)-2-oxo-4-(trifluoromethyl)imidazolidin-1-yl)ethyl)pyridin-2-yl)amino)ethyl)-1H-pyrazole-5-carboxamide;
1-methyl-N—((S)-1-((1r,4S)-4-methylcyclohexyl)-2-oxo-2-((4-((R)-1-((S)-2-oxo-4-(trifluoromethyl)imidazolidin-1-yl)ethyl)pyridin-2-yl)amino)ethyl)-1H-pyrazole-5-carboxamide;
1-isopropyl-N—((S)-1-((1r,4S)-4-methylcyclohexyl)-2-oxo-2-((4-((R)-1-((S)-2-oxo-4-(trifluoromethyl)imidazolidin-1-yl)ethyl)pyridin-2-yl)amino)ethyl)-1H-pyrazole-5-carboxamide;
N-((1S)-2-((4-(2-methoxy-1-(4,4,4-trifluorobutanamido)ethyl)pyridin-2-yl)amino)-1-((1r,4S)-4-methylcyclohexyl)-2-oxoethyl)-4-methyl-1,2,5-oxadiazole-3-carboxamide;
4-cyclopropyl-N-((1S)-2-((4-(2-methoxy-1-(4,4,4-trifluorobutanamido)ethyl)pyridin-2-yl)amino)-1-((1r,4S)-4-methylcyclohexyl)-2-oxoethyl)-1,2,5-oxadiazole-3-carboxamide;
1-ethyl-N-((1S)-2-((4-(2-methoxy-1-(4,4,4-trifluorobutanamido)ethyl)pyridin-2-yl)amino)-1-((1r,4S)-4-methylcyclohexyl)-2-oxoethyl)-1H-pyrazole-5-carboxamide;
N-((1S)-2-((4-(2-methoxy-1-(4,4,4-trifluorobutanamido)ethyl)pyridin-2-yl)amino)-1-((1r,4S)-4-methylcyclohexyl)-2-oxoethyl)-1-methyl-1H-pyrazole-5-carboxamide;
1-isopropyl-N-((1S)-2-((4-(2-methoxy-1-(4,4,4-trifluorobutanamido)ethyl)pyridin-2-yl)amino)-1-((1r,4S)-4-methylcyclohexyl)-2-oxoethyl)-1H-pyrazole-5-carboxamide;
2-(3-Cyanophenyl)-2,2-difluoro-N—((S)-2-((4-((S)-2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)acetamide;
2,2-difluoro-N—((S)-2-((4-((S)-2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)-2-(1-methyl-1H-pyrazol-5-yl)acetamide;
2-(3-chlorophenyl)-2,2-difluoro-N—((S)-2-((4-((S)-2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)acetamide;
2-(3,5-difluorophenyl)-2,2-difluoro-N—((S)-2-((4-((S)-2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)acetamide;
2,2-difluoro-N—((S)-2-((4-((S)-2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)-2-(pyrazin-2-yl)acetamide;
2,2-difluoro-N—((S)-2-((4-((S)-2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)-2-(6-methoxypyridin-3-yl)acetamide;
(S)—N-(4-((S)-2-methoxy-1-((S)-2-oxo-4-(trifluoromethyl)imidazolidin-1-yl)ethyl)pyridin-2-yl)-2-((1r,4S)-4-methylcyclohexyl)-2-(2-(5-methylisoxazol-3-yl)acetamido)acetamide;
2-fluoro-N—((S)-2-((4-((S)-2-methoxy-1-((S)-2-oxo-4-(trifluoromethyl)imidazolidin-1-yl)ethyl)pyridin-2-yl)amino)-1-((1r,4S)-4-methylcyclohexyl)-2-oxoethyl)-2-phenylacetamide;
2,2-difluoro-N—((S)-2-((4-((S)-2-methoxy-1-((S)-2-oxo-4-(trifluoromethyl)imidazolidin-1-yl)ethyl)pyridin-2-yl)amino)-1-((1r,4S)-4-methylcyclohexyl)-2-oxo ethyl)-3-methylbutanamide;
4-methyl-N—((S)-1-((1r,4S)-4-methylcyclohexyl)-2-oxo-2-((4-((R)-1-((S)-2-oxo-4-(trifluoromethyl)imidazolidin-1-yl)propyl)pyridin-2-yl)amino)ethyl)-1,2,5-oxadiazole-3-carboxamide;
4-cyclopropyl-N—((S)-1-((1r,4S)-4-methylcyclohexyl)-2-oxo-2-((4-((R)-1-((S)-2-oxo-4-(trifluoromethyl)imidazolidin-1-yl)propyl)pyridin-2-yl)amino)ethyl)-1,2,5-oxadiazole-3-carboxamide;
1-ethyl-N—((S)-1-((1r,4S)-4-methylcyclohexyl)-2-oxo-2-((4-((R)-1-((S)-2-oxo-4-(trifluoromethyl)imidazolidin-1-yl)propyl)pyridin-2-yl)amino)ethyl)-1H-pyrazole-5-carboxamide;
1-methyl-N—((S)-1-((1r,4S)-4-methylcyclohexyl)-2-oxo-2-((4-((R)-1-((S)-2-oxo-4-(trifluoromethyl)imidazolidin-1-yl)propyl)pyridin-2-yl)amino)ethyl)-1H-pyrazole-5-carboxamide;
1-isopropyl-N—((S)-1-((1r,4S)-4-methylcyclohexyl)-2-oxo-2-((4-((R)-1-((S)-2-oxo-4-(trifluoromethyl)imidazolidin-1-yl)propyl)pyridin-2-yl)amino)ethyl)-1H-pyrazole-5-carboxamide;
N—((S)-1-(3,3-difluorocyclobutyl)-2-((4-((S)-2-methoxy-1-((S)-2-oxo-4-(trifluoromethyl)imidazolidin-1-yl)ethyl)pyridin-2-yl)amino)-2-oxoethyl)-4-ethylisoxazole-3-carboxamide;
N—((S)-1-(3,3-difluorocyclobutyl)-2-((4-((S)-2-methoxy-1-((S)-2-oxo-4-(trifluoromethyl)imidazolidin-1-yl)ethyl)pyridin-2-yl)amino)-2-oxoethyl)-1-isopropyl-1H-pyrazole-5-carboxamide;
4-Cyclopropyl-N—((S)-1-((1 s,4R)-4-methylcyclohexyl)-2-oxo-2-((4-(((S)-2-oxo-4-(trifluoromethyl)imidazolidin-1-yl)methyl)pyridin-2-yl)amino)ethyl)-1,2,5-oxadiazole-3-carboxamide;
1-Methyl-N—((S)-1-((1s,4R)-4-methylcyclohexyl)-2-oxo-2-((4-(((S)-2-oxo-4-(trifluoromethyl)imidazolidin-1-yl)methyl)pyridin-2-yl)amino)ethyl)-1H-pyrazole-5-carboxamide;
N—((S)-1-((4-((S)-2-methoxy-1-((S)-2-oxo-4-(trifluoromethyl)imidazolidin-1-yl)ethyl)pyridin-2-yl)amino)-1-oxo-3-((1,1,1-trifluoro-2-methylpropan-2-yl)oxy)propan-2-yl)-4-methylisoxazole-3-carboxamide;
4-ethyl-N—((S)-1-((4-((S)-2-methoxy-1-((S)-2-oxo-4-(trifluoromethyl)imidazolidin-1-yl)ethyl)pyridin-2-yl)amino)-1-oxo-3-((1,1,1-trifluoro-2-methylpropan-2-yl)oxy)propan-2-yl)-1,2,5-oxadiazole-3-carboxamide;
3-ethyl-N—((S)-1-((4-((S)-2-methoxy-1-((S)-2-oxo-4-(trifluoromethyl)imidazolidin-1-yl)ethyl)pyridin-2-yl)amino)-1-oxo-3-((1,1,1-trifluoro-2-methylpropan-2-yl)oxy)propan-2-yl)isoxazole-4-carboxamide;
N—((S)-1-((4-((S)-2-methoxy-1-((S)-2-oxo-4-(trifluoromethyl)imidazolidin-1-yl)ethyl)pyridin-2-yl)amino)-1-oxo-3-((1,1,1-trifluoro-2-methylpropan-2-yl)oxy)propan-2-yl)-4-methyl-1,2,5-oxadiazole-3-carboxamide;
4-cyclopropyl-N—((S)-1-((4-((S)-2-methoxy-1-((S)-2-oxo-4-(trifluoromethyl) imidazolidin-1-yl)ethyl)pyridin-2-yl)amino)-1-oxo-3-((1,1,1-trifluoro-2-methylpropan-2-yl)oxy)propan-2-yl)-1,2,5-oxadiazole-3-carboxamide;
1-ethyl-N—((S)-1-((4-((S)-2-methoxy-1-((S)-2-oxo-4-(trifluoromethyl)imidazolidin-1-yl)ethyl)pyridin-2-yl)amino)-1-oxo-3-((1,1,1-trifluoro-2-methylpropan-2-yl)oxy)propan-2-yl)-1H-pyrazole-5-carboxamide;
N—((S)-1-((4-((S)-2-methoxy-1-((S)-2-oxo-4-(trifluoromethyl)imidazolidin-1-yl)ethyl)pyridin-2-yl)amino)-1-oxo-3-((1,1,1-trifluoro-2-methylpropan-2-yl)oxy)propan-2-yl)-1-methyl-1H-pyrazole-5-carboxamide;
1-isopropyl-N—((S)-1-((4-((S)-2-methoxy-1-((S)-2-oxo-4-(trifluoromethyl)imidazolidin-1-yl)ethyl)pyridin-2-yl)amino)-1-oxo-3-((1,1,1-trifluoro-2-methylpropan-2-yl)oxy)propan-2-yl)-1H-pyrazole-5-carboxamide;
4-methyl-N—((S)-1-oxo-1-((4-(((S)-2-oxo-4-(trifluoromethyl)imidazolidin-1-yl)methyl)pyridin-2-yl)amino)-3-((1,1,1-trifluoro-2-methylpropan-2-yl)oxy)propan-2-yl)-1,2,5-oxadiazole-3-carboxamide;
4-cyclopropyl-N—((S)-1-oxo-1-((4-(((S)-2-oxo-4-(trifluoromethyl)imidazolidin-1-yl)methyl)pyridin-2-yl)amino)-3-((1,1,1-trifluoro-2-methylpropan-2-yl)oxy)propan-2-yl)-1,2,5-oxadiazole-3-carboxamide;
1-methyl-N—((S)-1-oxo-1-((4-(((S)-2-oxo-4-(trifluoromethyl)imidazolidin-1-yl)methyl)pyridin-2-yl)amino)-3-((1,1,1-trifluoro-2-methylpropan-2-yl)oxy)propan-2-yl)-1H-pyrazole-5-carboxamide;
4-Cyclopropyl-N-(1-(4-fluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-1,2,5-oxadiazole-3-carboxamide;
1-ethyl-N-(1-(4-fluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-1H-pyrazole-5-carboxamide;
4-(2,2-difluoroethoxy)-N-(1-(4-fluorocyclohexyl)-2-((4-((S)-2-methoxy-1-((S)-2-oxo-4-(trifluoromethyl) imidazolidin-1-yl)ethyl)pyridin-2-yl)amino)-2-oxoethyl)isoxazole-3-carboxamide;
N-(1-(4-fluorocyclohexyl)-2-((4-((S)-2-methoxy-1-((S)-2-oxo-4-(trifluoromethyl) imidazolidin-1-yl)ethyl)pyridin-2-yl)amino)-2-oxoethyl)-4-(2,2,2-trifluoroethoxy)-1,2,5-oxadiazole-3-carboxamide;
N-(1-(4-fluorocyclohexyl)-2-((4-((S)-2-methoxy-1-((S)-2-oxo-4-(trifluoromethyl) imidazolidin-1-yl)ethyl)pyridin-2-yl)amino)-2-oxoethyl)-3-methylisoxazole-4-carboxamide;
N-(1-(4-fluorocyclohexyl)-2-((4-((S)-2-methoxy-1-((S)-2-oxo-4-(trifluoromethyl) imidazolidin-1-yl)ethyl)pyridin-2-yl)amino)-2-oxoethyl)-4-methylisoxazole-3-carboxamide;
4-ethyl-N-(1-(4-fluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-1,2,5-oxadiazole-3-carboxamide;
3-ethyl-N-(1-(4-fluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)isoxazole-4-carboxamide;
N-(1-(4-fluorocyclohexyl)-2-((4-((S)-2-methoxy-1-((S)-2-oxo-4-(trifluoromethyl) imidazolidin-1-yl)ethyl)pyridin-2-yl)amino)-2-oxoethyl)-4-methyl-1,2,5-oxadiazole-3-carboxamide;
N-(1-(4-fluorocyclohexyl)-2-((4-((S)-2-methoxy-1-((S)-2-oxo-4-(trifluoromethyl) imidazolidin-1-yl)ethyl)pyridin-2-yl)amino)-2-oxoethyl)-1-methyl-1H-pyrazole-5-carboxamide;
N-(1-(4-fluorocyclohexyl)-2-((4-((S)-2-methoxy-1-((S)-2-oxo-4-(trifluoromethyl) imidazolidin-1-yl)ethyl)pyridin-2-yl)amino)-2-oxoethyl)-1-isopropyl-1H-pyrazole-5-carboxamide;
4-Ethyl-N—((S)-1-((1r,4S)-4-fluorocyclohexyl)-2-oxo-2-((4-(((S)-2-oxo-4-(trifluoromethyl)imidazolidin-1-yl)methyl)pyridin-2-yl)amino)ethyl)-1,2,5-oxadiazole-3-carboxamide;
N—((S)-1-((1r,4S)-4-fluorocyclohexyl)-2-oxo-2-((4-(((S)-2-oxo-4-(trifluoromethyl)imidazolidin-1-yl)methyl)pyridin-2-yl)amino)ethyl)-4-methyl-1,2,5-oxadiazole-3-carboxamide;
4-cyclopropyl-N—((S)-1-((1r,4S)-4-fluorocyclohexyl)-2-oxo-2-((4-(((S)-2-oxo-4-(trifluoromethyl)imidazolidin-1-yl)methyl)pyridin-2-yl)amino)ethyl)-1,2,5-oxadiazole-3-carboxamide;
1-ethyl-N—((S)-1-((1r,4S)-4-fluorocyclohexyl)-2-oxo-2-((4-(((S)-2-oxo-4-(trifluoromethyl)imidazolidin-1-yl)methyl)pyridin-2-yl)amino)ethyl)-1H-pyrazole-5-carboxamide;
N—((S)-1-((1r,4S)-4-fluorocyclohexyl)-2-oxo-2-((4-(((S)-2-oxo-4-(trifluoromethyl)imidazolidin-1-yl)methyl)pyridin-2-yl)amino)ethyl)-1-methyl-1H-pyrazole-5-carboxamide;
4-Cyclopropyl-N-(1-(4-fluoro-4-methylcyclohexyl)-2-((4-((S)-2-methoxy-1-((S)-2-oxo-4-(trifluoromethyl)imidazolidin-1-yl)ethyl)pyridin-2-yl)amino)-2-oxoethyl)-1,2,5-oxadiazole-3-carboxamide;
N-(1-(4-fluoro-4-methylcyclohexyl)-2-((4-((S)-2-methoxy-1-((S)-2-oxo-4-(trifluoromethyl)imidazolidin-1-yl)ethyl)pyridin-2-yl)amino)-2-oxoethyl)-4-methyl-1,2,5-oxadiazole-3-carboxamide;
1-ethyl-N-(1-(4-fluoro-4-methylcyclohexyl)-2-((4-((S)-2-methoxy-1-((S)-2-oxo-4-(trifluoromethyl)imidazolidin-1-yl)ethyl)pyridin-2-yl)amino)-2-oxoethyl)-1H-pyrazole-5-carboxamide;
3-ethyl-N-((1S)-2-((4-((S)-2-methoxy-1-((S)-2-oxo-4-(trifluoromethyl)imidazolidin-1-yl)ethyl)pyridin-2-yl)amino)-2-oxo-1-(4-(trifluoromethyl)cyclohexyl)ethyl)isoxazole-4-carboxamide;
1-ethyl-N-((1S)-2-((4-((S)-2-methoxy-1-((S)-2-oxo-4-(trifluoromethyl)imidazolidin-1-yl)ethyl)pyridin-2-yl)amino)-2-oxo-1-(4-(trifluoromethyl)cyclohexyl)ethyl)-1H-pyrazole-5-carboxamide;
N-((1S)-2-((4-((S)-2-methoxy-1-((S)-2-oxo-4-(trifluoromethyl)imidazolidin-1-yl)ethyl)pyridin-2-yl)amino)-2-oxo-1-(4-(trifluoromethyl)cyclohexyl)ethyl)-1-methyl-1H-pyrazole-5-carboxamide;
1-isopropyl-N-((1S)-2-((4-((S)-2-methoxy-1-((S)-2-oxo-4-(trifluoromethyl)imidazolidin-1-yl)ethyl)pyridin-2-yl)amino)-2-oxo-1-(4-(trifluoromethyl)cyclohexyl)ethyl)-1H-pyrazole-5-carboxamide;
N—((S)-1-((S)-3,3-difluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-4-ethylisoxazole-3-carboxamide;
N—((S)-1-((S)-3,3-difluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-3-ethylisoxazole-4-carboxamide;
N—((S)-1-((S)-3,3-difluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-1-ethyl-1H-pyrazole-5-carboxamide;
N—((S)-1-((S)-3,3-difluorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-1-isopropyl-1H-pyrazole-5-carboxamide;
N-((1S)-1-(4-chlorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-1-ethyl-1H-pyrazole-5-carboxamide;
N-((1S)-1-(4-chlorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-1-methyl-1H-pyrazole-5-carboxamide;
N-((1S)-1-(4-chlorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-1-isopropyl-1H-pyrazole-5-carboxamide;
N-((1S)-1-(4-chlorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-3-methylisoxazole-4-carboxamide;
N-((1S)-1-(4-chlorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-4-ethylisoxazole-3-carboxamide;
N-((1S)-1-(4-chlorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-4-methyl-1,2,5-oxadiazole-3-carboxamide;
N-((1S)-1-(4-chlorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-4-ethyl-1,2,5-oxadiazole-3-carboxamide;
N-((1S)-1-(4-chlorocyclohexyl)-2-((4-((S)-2-methoxy-1-((S)-2-oxo-4-(trifluoromethyl)imidazolidin-1-yl)ethyl)pyridin-2-yl)amino)-2-oxoethyl)-3-ethylisoxazole-4-carboxamide;
N—((S)-1-((1R,3s,5S)-bicyclo[3.1.0]hexan-3-yl)-2-((4-((S)-2-methoxy-1-((S)-2-oxo-4-(trifluoromethyl)imidazolidin-1-yl)ethyl)pyridin-2-yl)amino)-2-oxoethyl)-5-ethylisoxazole-4-carboxamide;
N—((S)-1-((1R,3s,5S)-bicyclo[3.1.0]hexan-3-yl)-2-((4-((S)-2-methoxy-1-((S)-2-oxo-4-(trifluoromethyl)imidazolidin-1-yl)ethyl)pyridin-2-yl)amino)-2-oxoethyl)-5-isopropylisoxazole-4-carboxamide;
1-isopropyl-N—((S)-1-((1r,4S)-4-methylcyclohexyl)-2-oxo-2-((4-(((3S,5S)-2-oxo-5-(trifluoromethyl)pyrrolidin-3-yl)oxy)pyridin-2-yl)amino)ethyl)-1H-pyrazole-5-carboxamide;
3-ethyl-N—((S)-1-((1r,4S)-4-methylcyclohexyl)-2-oxo-2-((4-(((3S,5S)-2-oxo-5-(trifluoromethyl)pyrrolidin-3-yl)oxy)pyridin-2-yl)amino)ethyl)isoxazole-4-carboxamide;
1-ethyl-N—((S)-1-((1r,4S)-4-methylcyclohexyl)-2-oxo-2-((4-(((3S,5S)-2-oxo-5-(trifluoromethyl)pyrrolidin-3-yl)oxy)pyridin-2-yl)amino)ethyl)-1H-pyrazole-5-carboxamide; and
1-isopropyl-N—((S)-1-((1r,4S)-4-methylcyclohexyl)-2-oxo-2-((4-(((3R,5S)-2-oxo-5-(trifluoromethyl)pyrrolidin-3-yl)oxy)pyridin-2-yl)amino)ethyl)-1H-pyrazole-5-carboxamide.

30. A pharmaceutical composition comprising a compound according to any one of claims 1 to 29, or a pharmaceutically acceptable salt thereof, and one or more pharmaceutically acceptable excipients.

31. A compound according to any one of claims 1 to 29, or a pharmaceutically acceptable salt thereof, or a pharmaceutical composition according to claim 30, for use in therapy.

32. A compound according to any one of claims 1 to 29, or a pharmaceutically acceptable salt thereof, or a pharmaceutical composition according to claim 30, for use in the treatment of acute lung injury, Alzheimer's Disease, ankylosing spondylitis, axial spondyloarthritis and other spondyloarthropathies, arthritis, asthma (including severe asthma), atopic dermatitis, autoimmune diabetes, other autoimmune disorders, autoimmune thyroiditis, bone resorption, cancer (both solid tumours (such as melanomas, sarcomas, squamous cell carcinomas, transitional call cancers, and ovarian cancers) and hematologic malignancies; in particular acute myelogenous leukaemia, chronic lymphocytic leukemia, gastric cancer, and colon cancer), Castleman's disease, contact dermatitis, Crohn's Disease, chronic myelogenous leukemia, chronic obstructive pulmonary disease (COPD), coeliac disease, cystic fibrosis, dermatomyositis, discoid lupus erythematosus, eczema, enthesitis-related arthritis, endotoxic shock associated with infection, exophthalmos, fibrosing disorders including pulmonary fibrosis, gall bladder disease, giant cell arteritis, graft-versus-host disease, hepatoblastomas, hypochlorhydria, immune mediated inflammatory disorders of the central and peripheral nervous system such as multiple sclerosis and Guillain-Barr syndrome, Hidradenitis Suppurativa, inflammatory bowel disease, insulin dependent diabetes type 1, intravascular coagulation, irritable bowel syndrome, Lichen Planus, liver fibrosis, lupus nephritis, lyme arthritis, meningoencephalitis, myocarditis, meningoencephalitis, Necrobiosis Lipoidica Diabeticorum, osteoporosis, pancreatitis, Papulopustular Rosacea, Parkinson's disease, pelvic inflammatory disease, periodontitis, peritonitis, Peyronie's Disease, Pilonidal disease, psoriasis, psoriatic arthritis (PsA), Pyoderma Gangrenosum, renal fibrosis, rheumatoid arthritis, scleroderma or systemic sclerosis, stroke, surgical adhesions, systemic lupus erythematosus (SLE), systemic onset juvenile idiopathic arthritis (JIA), trauma (surgery), transplant rejection, Type I diabetes, ulcerative colitis, uveitis, or vasculitis.

33. A method of treating a disease or disorder selected from acute lung injury, Alzheimer's Disease, ankylosing spondylitis, axial spondyloarthritis and other spondyloarthropathies, arthritis, asthma (including severe asthma), atopic dermatitis, autoimmune diabetes, other autoimmune disorders, autoimmune thyroiditis, bone resorption, cancer (both solid tumours (such as melanomas, sarcomas, squamous cell carcinomas, transitional call cancers, and ovarian cancers) and hematologic malignancies; in particular acute myelogenous leukaemia, chronic lymphocytic leukemia, gastric cancer, and colon cancer), Castleman's disease, contact dermatitis, Crohn's Disease, chronic myelogenous leukemia, chronic obstructive pulmonary disease (COPD), coeliac disease, cystic fibrosis, dermatomyositis, discoid lupus erythematosus, eczema, enthesitis-related arthritis, endotoxic shock associated with infection, exophthalmos, fibrosing disorders including pulmonary fibrosis, gall bladder disease, giant cell arteritis, graft-versus-host disease, hepatoblastomas, hypochlorhydria, immune mediated inflammatory disorders of the central and peripheral nervous system such as multiple sclerosis and Guillain-Barr syndrome, Hidradenitis Suppurativa, inflammatory bowel disease, insulin dependent diabetes type 1, intravascular coagulation, irritable bowel syndrome, Lichen Planus, liver fibrosis, lupus nephritis, lyme arthritis, meningoencephalitis, myocarditis, meningoencephalitis, Necrobiosis Lipoidica Diabeticorum, osteoporosis, pancreatitis, Papulopustular Rosacea, Parkinson's disease, pelvic inflammatory disease, periodontitis, peritonitis, Peyronie's Disease, Pilonidal disease, psoriasis, psoriatic arthritis (PsA), Pyoderma Gangrenosum, renal fibrosis, rheumatoid arthritis, scleroderma or systemic sclerosis, stroke, surgical adhesions, systemic lupus erythematosus (SLE), systemic onset juvenile idiopathic arthritis (JIA), trauma (surgery), transplant rejection, Type I diabetes, ulcerative colitis, uveitis, and vasculitis, the method comprising administering to a patient in need thereof a therapeutically effective amount of a compound according to any one of claims 1 to 29, or a pharmaceutically acceptable salt thereof, or a pharmaceutical composition according to claim 30.
